SEQUENCE LISTING (23 sequences)

SEQ ID NO: 1 - protein, 15 aa, Artificial
  AviTag
GLNDIFEAQKIEWHE

SEQ ID NO: 2 - protein, 26 aa, Artificial
  Calmodulin-tag
KRRWKKNFIAVSAANRFKKISSSGAL

SEQ ID NO: 3 - protein, 6 aa, Artificial
  polyglutamate tag
EEEEEE

SEQ ID NO: 4 - protein, 13 aa, Artificial
  E-tag
GAPVPYPDPLEPR

SEQ ID NO: 5 - protein, 8 aa, Artificial
  FLAG-tag
DYKDDDDK

SEQ ID NO: 6 - protein, 9 aa, Artificial
  HA-tag
YPYDVPDYA

SEQ ID NO: 7 - protein, 6 aa, Artificial
  His-tag
HHHHHH

SEQ ID NO: 8 - protein, 10 aa, Artificial
  Myc-tag
EQKLISEEDL

SEQ ID NO: 9 - protein, 18 aa, Artificial
  NE-tag
TKENPRSNQEESYDDNES

SEQ ID NO: 10 - protein, 15 aa, Artificial
  S-tag
KETAAAKFERQHMDS

SEQ ID NO: 11 - protein, 38 aa, Artificial
  SBP-tag
MDEKTTGWRGGHVVEGLAGELEQLRARLEHHPQGQREP

SEQ ID NO: 12 - protein, 13 aa, Artificial
  Softag 1
SLAELLNAGLGGS

SEQ ID NO: 13 - protein, 8 aa, Artificial
  Softag 3
TQDPSRVG

SEQ ID NO: 14 - protein, 8 aa, Artificial
  Strep-tag II
WSHPQFEK

SEQ ID NO: 15 - protein, 6 aa, Artificial
  TC tag
CCPGCC

SEQ ID NO: 16 - protein, 14 aa, Artificial
  V5 tag
GKPIPNPLLGLDST

SEQ ID NO: 17 - protein, 11 aa, Artificial
  VSV-tag
YTDIEMNRLGK

SEQ ID NO: 18 - protein, 8 aa, Artificial
  Xpress tag
DLYDDDDK

SEQ ID NO: 19 - protein, 16 aa, Artificial
  Isopeptag
TDKDMTITFTNKKDAE

SEQ ID NO: 20 - protein, 13 aa, Artificial
  SpyTag
AHIVMVDAYKPTK

SEQ ID NO: 21 - protein, 12 aa, Artificial
  SnoopTag
KLGDIEFIKVNK

SEQ ID NO: 22 - protein, 264 aa, Artificial
  eGFP C70M S147C
MGSSHHHHHHSSGLVPRGSHMGSIQMVSKGEELFTGVVPILVELDGDVNGHKFSVSGEGEGDATYGKLTLKFICTTGKLPVPWPTLVTTLTYGVQMFSRYPDHMKQHDFFKSAMPEGYVQERTIFFKDDGNYKTRAEVKFEGDTLVNRIELKGIDFKEDGNILGHKLEYNYNCHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - protein, 18 aa, Artificial
  tandem mass spectrometry analysis of in-gel trypsin-digested and labeled eGFP
LEYNYNCPVHNVYIMADK

(19) United States
(12) Patent Application Publication
(10) Pub. No.: US 2024/0350655 A1
(43) Pub. Date: Oct. 24, 2024

(54) THIOL-CONJUGATION WITH UNSATURATED PHOSPHORUS(V) COMPOUNDS

(71) Applicant: FORSCHUNGSVERBUND BERLIN E.V., Berlin (DE)

(72) Inventors: Christian Ewald STIEGER, Berlin (DE); Christian Peter Richard HACKENBERGER, Berlin (DE); Marc-André KASPER, Muenchen (DE); Philipp OCHTROP, Berlin (DE)

(21) Appl. No.: 18/556,602

(22) PCT Filed: Apr. 22, 2022
(86) PCT No.: PCT/EP2022/060691

§ 371 Date: Oct. 20, 2023

(30) Foreign Application Priority Data
Apr. 23, 2021 (EP) 21170097.6

(51) Int. Cl. A61K 47/68; A61K 47/54; C07F 9/32; C07F 9/6518; C07F 9/6524; C07F 9/653; C07F 9/6558; C07F 9/6561

(52) U.S. Cl. CPC A61K 47/6851; A61K 47/548; A61K 47/68031; C07F 9/3217; C07F 9/6518; C07F 9/6524; C07F 9/65324; C07F 9/65583; C07F 9/65586; C07F 9/6561

(57) ABSTRACT

Disclosed are novel conjugates and methods for the preparation thereof. One of the methods for the preparation of a conjugate comprises a step of: acting a compound of formula (I), with a thiol-containing molecule of formula (II), wherein represents an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system; resulting in a compound of formula (III).

### CROSS-REFERENCE TO RELATED APPLICATIONS

The present application claims the benefit of priority of European Patent Application No. 21170097.6 filed 23 Apr. 2021, the content of which is hereby incorporated by reference in its entirety for all purposes.

### REFERENCE TO AN ELECTRONIC SEQUENCE LISTING

The contents of the electronic sequence listing (350066_407USPC_SeqListing.txt; Size: 7,332 bytes; and Date of Creation: Oct. 17, 2023) is herein incorporated by reference in its entirety.

### TECHNICAL FIELD OF THE INVENTION

The present invention relates to methods of preparing compounds and conjugates of antibody molecules via thiol-conjugation with unsaturated phosphorus(V) compounds. The present invention also relates to compounds and conjugates of antibody molecules, as described herein.

### BACKGROUND OF THE INVENTION

Site-selective modification of biomolecules with small chemical handles, such as fluorophores or bioactive compounds, enables a plethora of applications in life sciences and pharmacology (E. A. Hoyt, P. M. S. D. Cal, B. L. Oliveira, G. J. L. Bemardes, Nat. Rev. Chem. 2019, 3, 147-171; D. Schumacher, C. P. R. Hackenberger, Curr. Opin. Chem. Biol. 2014, 22, 62-69). Most commonly, electrophilic reagents that target nucleophilic amino acids like lysine or cysteine (Cys) are employed (E. A. Hoyt, P. M. S. D. Cal, B. L. Oliveira, G. J. L. Bemardes, Nat. Rev. Chem. 2019, 3, 147-171; S. B. Gunnoo, A. Madder, ChemBioChem 2016, 17, 529-553; P. Ochtrop, C. P. R. Hackenberger, Curr. Opin. Chem. Biol. 2020, 58, 28-36). Other than that, also most other nucleophilic amino-acids can be selectively addressed, however these methods are less well established (S. Jia, D. He, C. J. Chang, 2019, DOI 10.1021/jacs.8b11912; J. C. Vantourout, S. Rao Adusumalli, K. W. Knouse, D. T. Flood, A. Ramirez, N. M. Padial, A. Istrate, K. Maziarz, J. N. deGruyter, R. R. Merchant, J. X. Qiao, M. A. Schmidt, M. J. Deery, M. D. Eastgate, P. E. Dawson, alo J. L Bemardes, P. S. Baran, J. Am. Chem. Soc 2020, 142, 46; S. Sato, M. Matsumura, T. Kadonosono, S. Abe, T. Ueno, H. Ueda, H. Nakamura, Bioconjug. Chem. 2020, 31, 1417-1424; M. T. Taylor, J. E. Nelson, M. G. Suero, M. J. Gaunt, Nature 2018, 562, 563-568; D. Hwang, N. Nilchan, A. R. Nanna, X. Li, M. D. Cameron, W. R. Roush, H. J. Park, C. Rader, Cell Chem. Biol. 2019, 26, 1229-1239.e9). Additionally, the incorporation of non-natural amino acids equipped with azides, trans-cyclooctenes or tetrazines allows the biorthogonal modification of peptides and proteins (K. Lang, J. W. Chin, ACS Chem. Biol. 2014, 9, 16-20; X. Chen, Y. W. Wu, Org. Biomol. Chem. 2016, 14, 5417-5439).

Among the proteinogenic amino acids, Cys offers several advantages when it comes to protein modification. First, the enhanced nucleophilicity of the sulfhydryl group simplifies site-selective modification. Moreover, its relatively low abundance on accessible protein surfaces often allows selective mono-functionalisation of proteins (S. B. Gunnoo, A. Madder, ChemBioChem 2016, 17, 529-553). Therefore, several approaches to selectively label Cys on proteins have been developed. Among them, thio-Michael addition to maleimides remains the most widely used method. Although this reaction offers rapid kinetics at near neutral pH, the generated thiosuccinimide linkage is inherently instable in the presence of external thiols, caused by retro-Michael addition (B. Q. Shen, K. Xu, L. Liu, H. Raab, S. Bhakta, M. Kenrick, K. L. Parsons-Reponte, J. Tien, S. F. Yu, E. Mai, D. Li, J. Tibbitts, J. Baudys, O. M. Saad, S. J. Scales, P. J. McDonald, P. E. Hass, C. Eigenbrot, T. Nguyen, W. A. Solis, R. N. Fuji, K. M. Flagella, D. Patel, S. D. Spencer, L. A. Khawli, A. Ebens, W. L. Wong, R. Vandlen, S. Kaur, M. X. Sliwkowski, R. H. Scheller, P. Polakis, J. R. Junutula, Nat. Biotechnol. 2012, 30, 184-189).

In addition to the modification of single Cys-residues with a specific payload, peptide- or protein-modifications using double reactive Cys-specific reagents have been described in the literature (M. E. B. Smith, F. F. Schumacher, C. P. Ryan, L. M. Tedaldi, D. Papaioannou, G. Waksman, S. Caddick, J. R. Baker, J. Am. Chem. Soc. 2010, 132, 1960-1965; A. L. Baumann, S. Schwagerus, K. Broi, K. Kemnitz-Hassanin, C. E. Stieger, N. Trieloff, P. Schmieder, C. P. R. Hackenberger, J. Am. Chem. Soc. 2020, 142, 9544-9552; Y. Zhang, C. Zang, G. An, M. Shang, Z. Cui, G. Chen, Z. Xi, C. Zhou, Nat. Commun. 2020, 11, 1-10; C. Canovas, M. Moreau, C. Bemhard, A. Oudot, M. Guillemin, F. Denat, V. Goncalves, Angew. Chemie Int. Ed. 2018, 57, 10646-10650; T. Wang, A. Riegger, M. Lamla, S. Wiese, P. Oeckl, M. Otto, Y. Wu, S. Fischer, H. Barth, S. L. Kuan, T. Weil, Chem. Sci. 2016, 7, 3234-3239; D. L. Paterson, J. U. Flanagan, P. R. Shepherd, P. W. R. Harris, M. A. Brimble, Chem.-A Eur. J. 2020, 26, 10826-10833). However, similar to normal maleimides, linkages generated with such reagents are often instable towards reducing conditions or excess of small thiols (M. E. B. Smith, F. F. Schumacher, C. P. Ryan, L. M. Tedaldi, D. Papaioannou, G. Waksman, S. Caddick, J. R. Baker, J. Am. Chem. Soc. 2010, 132, 1960-1965; Y. Zhang, C. Zang, G. An, M. Shang, Z. Cui, G. Chen, Z. Xi, C. Zhou, Nat. Commun. 2020, 11, 1-10). These bis-functional reagents can be used for the intermolecular crosslinking of two Cys-containing proteins (Y. Zhang, C. Zang, G. An, M. Shang, Z. Cui, G. Chen, Z. Xi, C. Zhou, Nat. Commun. 2020, 11, 1-10; S. J. Walsh, S. Omarjee, W. R. J. D. Galloway, T. T.-L. Kwan, H. F. Sore, J. S. Parker, M. Hyvönen, J. S. Carroll, D. R. Spring, Chem. Sci. 2019, 10, 694-700; M. E. B. Smith, F. F. Schumacher, C. P. Ryan, L. M. Tedaldi, D. Papaioannou, G. Waksman, S. Caddick, J. R. Baker, J. Am. Chem. Soc. 2010, 132, 1960-1965). Alternatively, in a process commonly referred to as disulfide-rebridging, cysteines can be converted following reduction and subsequent reaction with a bis-electrophile, an approach that is particularly useful for antibody-functionalisation. In addition to the site-selective modification with a defined stoichiometry, the covalent non-reductive linkage between the antibody chains has been shown to increase its thermal stability (S. Sun, P. Akkapeddi, M. C. Marques, N. Martinez-Sáez, V. M. Torres, C. Cordeiro, O. Boutureira, G. J. L. Bemardes, Org. Biomol. Chem. 2019, 17, 2005-2012; C. Bahou, E. A. Love, S. Leonard, R. J. Spears, A. Maruani, K. Armour, J. R. Baker, V. Chudasama, Bioconjug. Chem. 2019, 30, 1048-1054). Recently, reagents based on functionalized dibromomaleimides (J. P. M. Nunes, M. Morais, V. Vassileva, E. Robinson, V. S. Rajkumar, M. E. B. Smith, R. B. Pedley, S. Caddick, J. R. Baker, V. Chudasama, Chem. Commun. 2015, 51, 10624-10627), divinylpyrimidines (S. J. Walsh, S. Omarjee, W. R. J. D. Galloway, T. T.-L. Kwan, H. F. Sore, J. S. Parker, M. Hyvönen, J. S. Carroll, D. R. Spring, Chem. Sci. 2019, 10, 694-700) and vinylsulfones (G. Badescu, P. Bryant, M. Bird, K. Henseleit, J. Swierkosz, V. Parekh, R. Tommasi, E. Pawlisz, K. Jurlewicz, M. Farys, N. Camper, X. Sheng, M. Fisher, R. Grygorash, A. Kyle, A. Abhilash, M. Frigerio, J. Edwards, A. Godwin, Bioconjug. Chem. 2014, 25, 1124-1136) have been used to generate antibody-drug-conjugates (ADCs) with a precise drug-to-antibody-ratio of four.

Previously, unsaturated phosphonamidates and phosphonothioates have been introduced as electrophilic reagents for chemoselective Cys modification, yielding highly stable conjugates (A. L. Baumann, S. Schwagerus, K. Broi, K. Kemnitz-Hassanin, C. E. Stieger, N. Trieloff, P. Schmieder, C. P. R. Hackenberger, J. Am. Chem. Soc. 2020, 142, 9544-9552; M. Kasper, M. Glanz, A. Stengl, M. Penkert, S. Klenk, T. Sauer, D. Schumacher, J. Helma, E. Krause, M. C. Cardoso, H. Leonhardt, C. P. R. Hackenberger, Angew. Chemie Int. Ed. 2019, 58, 11625-11630; M. A. Kasper, M. Glanz, A. Oder, P. Schmieder, J. P. Von Kries, C. P. R. Hackenberger, Chem. Sci. 2019, 10, 6322-6329; WO 2018/041985; WO 2019/170710). These compounds can be generated from vinyl- or alkynyl-phosphonites and azides or electrophilic disulfides, respectively. Moreover, electrophilic P(V)-reagents have been employed in peptide-stapling (M. A. Kasper, M. Glanz, A. Oder, P. Schmieder, J. P. Von Kries, C. P. R. Hackenberger, Chem. Sci. 2019, 10, 6322-6329), protein-protein conjugation (A. L. Baumann, S. Schwagerus, K. Broi, K. Kemnitz-Hassanin, C. E. Stieger, N. Trieloff, P. Schmieder, C. P. R. Hackenberger, J. Am. Chem. Soc. 2020, 142, 9544-9552; M. Kasper, M. Glanz, A. Stengl, M. Penkert, S. Klenk, T. Sauer, D. Schumacher, J. Helma, E. Krause, M. C. Cardoso, H. Leonhardt, C. P. R. Hackenberger, Angew. Chemie Int. Ed. 2019, 58, 11625-11630) and in the production of efficacious ADCs (M. Kasper, A. Stengl, P. Ochtrop, M. Gerlach, T. Stoschek, D. Schumacher, J. Helma, M. Penkert, E. Krause, H. Leonhardt, C. P. R. Hackenberger, Angew. Chemie Int. Ed. 2019, 58, 11631-11636; M. Kasper, M. Gerlach, A. F. L. Schneider, C. Groneberg, P. Ochtrop, S. Boldt, D. Schumacher, J. Helma, H. Leonhardt, M. Christmann, C. P. R. Hackenberger, ChemBioChem 2020, 21, 113-119).

It is an object of the present invention to provide further methods of preparing conjugates, and to provide further conjugates.

### SUMMARY OF THE INVENTION

The present invention, in one aspect, relates to a method of preparing a compound of formula (III) comprising a step of
reacting a compound of formula (I)

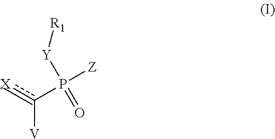

wherein
[symbol] represents a triple bond or a double bond;
V is absent when [symbol] is a triple bond; or
V represents H or C₁-C₈-alkyl when [symbol] is a double bond;
X represents R₃—C when [symbol] is a triple bond; or
X represents

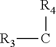

when [symbol] is a double bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl; and
Z represents a residue bound to the phosphorus via a carbon atom and comprising a group ●, wherein ● represents an optionally substituted aliphatic or optionally substituted aromatic residue;
with a thiol-containing molecule of formula (II)

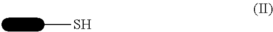

wherein [symbol] represents an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system;
resulting in a compound of formula (III)

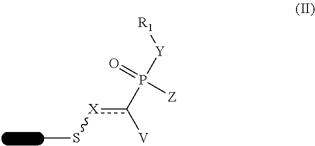

wherein
[symbol] represents a double bond when [symbol] in a compound of formula (I) represents a triple bond; or
[symbol] represents a bond when [symbol] in a compound of formula (I) represents a double bond;
V is absent when [symbol] is a double bond; or
V represents H or C₁-C₈-alkyl when [symbol] is a bond;
X represents R₃—C when [symbol] is a double bond; or
X represents

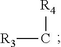

when [symbol] is a bond; and
[symbol] R₁, R₃, R₄, Y and Z are as defined for the compounds of formula (I) and formula (11).

The present invention also relates to a method wherein a compound of formula (L)

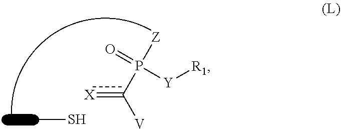

wherein the

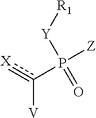

and the [symbol] S H are in the same molecule as indicated by the arc connecting the Z and the [symbol] , is reacted to give a compound of formula (IIIa):

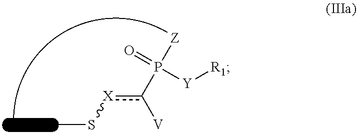

wherein [symbol] represents a bond if [symbol] in a compound of formula (L) represents a double bond, and X represent

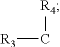

or
[symbol] represents a double bond if [symbol] in a compound of formula (L) represents a triple bond, and X represents R₃—C; and
[symbol] R₁, R₃, R₄ V, Y and Z are as defined herein.

The present invention also relates to a method of preparing a conjugate of an antibody molecule, said method comprising:
reducing at least one disulfide bridge of an antibody molecule in the presence of a reducing agent; and
reacting said antibody molecule with a compound of formula (IV*)

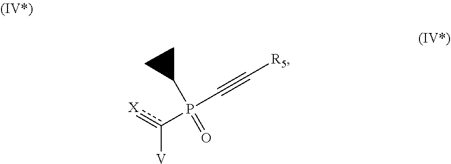

[symbol] represents a triple bond or a double bond;
V is absent when [symbol] is a triple bond; or
V represents H or C₁-C₈-alkyl when [symbol] is a double bond;
X represents R₃—C when [symbol] is a triple bond; or
X represents

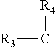

when [symbol] is a double bond;
▴ represents

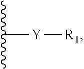

wherein [symbol] indicates the attachment point to the phosphorus; or
▴ represents Z;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
R₅ represents H or C₁-C₈-alkyl; and
Z represents a residue bound to the phosphorus via a carbon atom an comprising a group ●, wherein ● represents an optionally substituted aliphatic of optionally substituted aromatic residue
resulting in a conjugate of an antibody molecule comprising at least one moiety of formula (V)

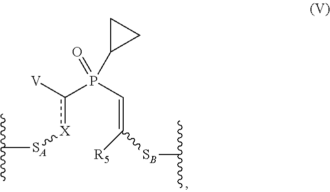

wherein S_{A} and S_{B} are each sulfur atoms of a chain of the antibody molecule;
[symbol] represents a double bond when [symbol] in a compound of formula (IV*) represents a triple bond; or
[symbol] represents a bond when [symbol] in a compound of formula (IV*) represents a double bond;
V is absent when [symbol] is a double bond; or
V represents H or C₁-C₈-alkyl when [symbol] is a bond;
X represents R₃—C when [symbol] is a double bond; or
X represents

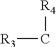

when [symbol] is a bond;
▴ represents

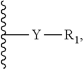

wherein [symbol] indicates the attachment point to the phosphorus; or
▴ represents Z; and
wherein R₁, R₃, R₄, R₅, Y and Z are as defined for the compound of formula (IV*).

The present invention also relates to a compound of formula (I)

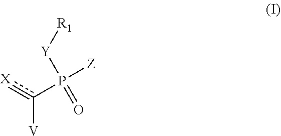

wherein
[symbol] represents a triple bond or a double bond;
V is absent when [symbol] is a triple bond; or
V represents H or C₁-C₈-alkyl when [symbol] is a double bond;
X represents R₃—C when [symbol] is a triple bond; or
X represents

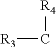

when [symbol] is a double bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl; and
Z represents a residue bound to the phosphorus via a carbon atom and comprising a group ●, wherein ● represents an optionally substituted aliphatic or optionally substituted aromatic residue.

The present invention also relates to a compound of formula (III)

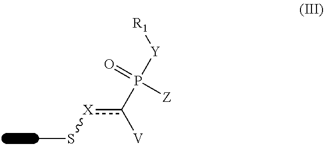

wherein
[symbol] represents a double bond; or
[symbol] represents a bond;
V is absent when [symbol] is a double bond; or
V is H or C₁-C₈-alkyl when [symbol] is a bond;
X represents R₃—C when [symbol] is a double bond; or
X represents

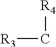

when [symbol] is a bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
Z represents a residue bound to the phosphorus via a carbon atom and comprising a group ●, wherein ● represents an optionally substituted aliphatic or optionally substituted aromatic residue; and
[symbol] represents an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system.

The invention also relates to a compound of formula (IIIa)

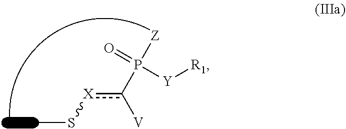

wherein
[symbol] represents a double bond; or
[symbol] represents a bond;
V is absent when [symbol] is a double bond; or
V is H or C₁-C₈-alkyl when [symbol] is a bond;
X represents R₃—C when [symbol] is a double bond; or
X represents

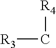

when [symbol] is a bond; and
R₁, R₃, R₄, V, X, Y, Z and [symbol] are as defined herein above and below, in particular as defined with regard to a compound of formula (III).

The present invention also relates to a compound of formula (IV)

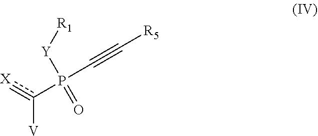

wherein R₁, R₅, [symbol] , V, X and Y are as defined herein for any one of the methods, compounds and/or conjugates.

The present invention also relates to a compound of formula (IV*)

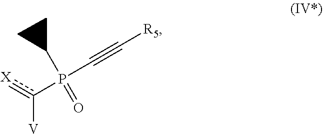

wherein
[symbol] represents a triple bond or a double bond;
V is absent when [symbol] is a triple bond; or
V represents H or C₁-C₈-alkyl when [symbol] is a double bond;
X represents R₃—C when [symbol] is a triple bond;
X represents

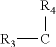

when [symbol] is a double bond;
▴ represents

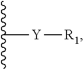

wherein [symbol] indicates the attachment point to the phosphorus; or
▴ represents Z;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
R₅ represents H or C₁-C₈-alkyl; and
Z represents a residue bound to the phosphorus via a carbon atom and comprising a group ●, wherein ● represents an optionally substituted aliphatic or optionally substituted aromatic residue.

The present invention also relates to a conjugate of an antibody molecule comprising at least one moiety of formula (V)

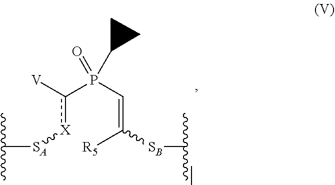

wherein S_{A} and S_{B} are each sulfur atoms of a chain of the antibody molecule;
[symbol] represents a double bond; or
[symbol] represents a bond;
V is absent when [symbol] is a double bond; or
V represents H or C₁-C₈-alkyl when [symbol] is a bond;
X represents R₃—C when [symbol] is a double bond;
X represents

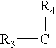

when [symbol] is a bond;
▴ represents

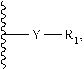

wherein [symbol] indicates the attachment point to the phosphorus; or
▴ represents Z;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
R₅ represents H or C₁-C₈-alkyl; and
Z represents a residue bound to the phosphorus via a carbon atom and comprising a group ●, wherein ● represents an optionally substituted aliphatic or optionally substituted aromatic residue.

### BRIEF DESCRIPTION OF THE FIGURES

FIG. 1 shows the development of substituted diethynyl-phosphinates as reagents for selective thiol-thiol bio-conjugation and rebridging of native disulphides, e.g. in therapeutic antibodies.

FIG. 2 shows the E/Z-selectivity of the thiol addition with diethynyl-phosphinates. NMR analysis of the isolated compounds allowed to identify the different isomers based on the characteristic coupling constants of the alkene-protons and their comparison to previously characterized thioladducts (Kasper, M.; Glanz, M.; Stengl, A.; Penkert, M.; Klenk, S.; Sauer, T.; Schumacher, D.; Helma, J.; Krause, E.; Cardoso, M. C.; Leonhardt, H.; Hackenberger, C. P. R. Cysteine-Selective Phosphonamidate Electrophiles for Modular Protein Bioconjugations. Angew. Chemie Int. Ed. 2019, 58 (34), 11625-11630. https://doi.org/10.1002/anie.201814715; Mikolajczyk, M.; Costisella, B.; Grzejszczak, S. Organosulphur Compounds-XXIX. Synthesis and Pummerer Rearrangement of β-Phosphoryl Sulphoxides. Tetrahedron1983, 39 (7), 1189-1193. https://doi.org/10.1016/S0040-4020(01)91883-6).

FIG. 3 shows isomerization of the formed thiol-adduct from Z/Z to E/E The isolated Z/Z-product (1-EtSH) was placed into an NMR-tube and dissolved in CDCl₃. ¹H- and ³¹P NMR were recorded over a period of four days to monitor the isomerization into the E/E-form. FIG. 3a shows a schematic representation of the isomerization process. FIG. 3b shows ¹H-NMR scans illustrating the stepwise isomerization. FIG. 3c shows Integrals of the ³¹P-NMR signals corresponding to the different isomers over time.

FIG. 4 shows the synthesis of quenched FRET-pairs F1-F3. Synthetic procedure for the generation of quenched fluorophore pairs: Quenched FRET-Pair 1 was synthesized from peptide 2 and excess (10 eq.) phosphinate 1 in PBS (pH 7.4). After purification of the intermidiate, it was reacted with 1.2 eq. EDANS-thiol in PBS. F1 was purified via semi-preperaive HPLC (1.67 mg, 86%). Quenched FRET-Pair 2 was synthesized analogously to F1, only phosphinate 2 was used as a linker. F2 was purified via semi-preperaive HPLC (1.94 mg, 91%). Quenched FRET-Pair 3 was synthesized from 2 eq. peptide 2 and phosphinate 2 (1 eq.) in PBS. Subsequently, EDANS-azide was conjugated to the phosphinate side-chain using CuBr (10 mol %) as catalyst. F3 was purified via semi-preperaive HPLC (2.13 mg, 78%).

FIG. 5a shows a FRET-quenching assay to investigate the stability of the thiol-conjugates. FIG. 5b and FIG. 5c show the observed EDANS-fluorescence for constructs F1 & F2 in PBS, PBS supplemented with glutathione, human serum and in 0.1M aq. NaOH over 72 h. FIG. 5d shows a General scheme for the site-selective protein-modification using dietnynyl-phosphinates and deconvoluted intact-protein-MS spectra of successfully labelled proteins. FIG. 5e shows that conjugation of a cell penetrating R₁₀-peptide to mCherry-5 allows delivery of mCherry into living cells with nucleolar localization and co-localization of mCherry with NBD.

FIG. 6 shows the stability testing of diethynyl-phosphinate conjugates using a fluorophore-quencher based assay to investigate the stability of phosphinate-thiol adducts. FIG. 6a and FIG. 6b show the structure of the phosphinate linked dye-quencher conjugates and principles of the fluorescence-quencher based readout. FIG. 6c shows fluorescence measurements for conjugates F1-F3. Stability studies of the Dabcyl-EDANS adducts were conducted in 96-well plate (Corning 3615, black with clear, flat bottom) at least in triplicates. 5 μl of a 200 μM Stock solution of the Dabcyl-EDANS conjugates and 95 μl of the respective test solutions were added to each well. Human serum was purchased from Sigma Aldrich. Glutathione was dissolved at a concentration of 10 mM in PBS and the pH was adjusted to 7.4. 0.1 mM NaOH studies were conducted at 200 μM, neutralized to pH 7 and diluted to 10 μM before fluorescence measurements. Fluorescence was measured on a Tecan Safire plate reader. Excitation: 360 nm, emission: 508 nm, bandwidth: 5 nm at 20° C.

FIG. 7 shows eGFP-labeling with phosphinate 1 and determination of the effect of labeling proteins with Diethynyl phosphinates on the secondary structure. FIG. 7a shows labeling of eGFP (C70M S147C) with 10 eq. 1 in PBS (30 min, r.t.) followed by purification via 0.5 mL Zeba™ Spin Desalting Columns with 7K MWCO (Thermo Fisher Scientific, USA) into phosphate-buffer (20 mM, pH 7.5). Complete labeling was verified via intact-protein MS. FIG. 7b shows CD and fluorescence spectra recorded after the protein was diluted to a concentration of 5 μM. Comparison to spectra obtained from non-modified eGFP showed no significant differences. This indicates, that the secondary structure of the protein is not affected upon labeling. FIG. 7c shows a tandem mass spectrometry analysis of in-gel trypsin-digested, labeled eGFP verified that only cysteine is labeled.

FIG. 8 shows estimation of the reaction kinetics of diethynyl phosphinates with proteins. Determination of the second-order rate constant of the reaction between eGFP (C70M S147C) and phosphinate 2. FIG. 8a shows the Reaction conditions. Reactions were carried out in triplicate. 90 μl eGFP (0.1 mM) were mixed with 10 μl of a 0.9 mM solution of phosphinate 2 in DMSO. Samples were drawn after 60, 90, 150, 210 and 300 minutes and analyzed via intact protein-MS. FIG. 8b shows the mathematic consideration for the determination of a second order rate constant with equal concentrations of the two reactants. FIG. 8c shows the concentration of eGFP over time. Calculated by the intensity of the deconvoluted mass in relation to the internal standard eGFP (C70M). FIG. 8d shows 1/c over time. The slope corresponds to the second order rate constant. Shown are mean and error of three independent measurements.

FIG. 9 shows diethynyl phosphinates as linker-molecules for the attachment of cell-penetrating peptides to proteins. FIG. 9a shows a schematic illustration of the generation of the eGFP-R₁₀-conjugate. FIG. 9b shows Fluorescence imaging of HeLa-cells after incubation with eGFP alone and eGFP-R₁₀ following the procedure of Schneider et al. (Schneider, Anselm F. L.; Kithil, Marina; Cardoso, M. Cristina; Lehmann, Martin; Hackenberger, Christian P. R.; Cellular uptake of Large Biomolecules Enabled by Cell-surface-reactive Cell-penetrating Peptide Additives, Nat. Chem. (2021) accepted Manuscript https://doi.org/10.1038/s41557-021-00661-x.). HeLa Kyoto cells were grown at 37° C. in a humidified atmosphere with 5% CO₂ in DMEM 4.5 g/L glucose with 10% fetal bovine serum (FBS). 15′000 HeLa Kyoto cells were seeded per well of a 8-well ibidi μ-slide. Cells were left to adhere and grow for 24 hours at 37° C. and 5% CO₂. Cells were washed twice with Fluorobrite DMEM without FBS and incubated with 10 μM eGFP-1-R₁₀ and additives (10 μM TNB-R₁₀) in Fluorobrite DMEM without FBS. Cells were incubated for 1 h at 37° C. and 5% CO₂. Cells were washed three times with Fluorobrite DMEM with 20 mM glutamine and 10% FBS. The cells were then covered with Fluorobrite DMEM with 20 mM glutamine and 10% FBS containing Hoechst stain (Hoechst 33342). Live cell microscopy images of the eGFP uptake experiments were acquired on Nikon-CSU spinning disc microscope with a CSU-X1 (Andor) and live cell incubation chamber (OKOlab). All live cell images were acquired using a PlanApo 60× NA 1.4 oil objective (Nikon) and an EMCCD (AU888, Andor). Brightfield images were acquired along with fluorescence images. Standard laser, a quad Dicroic (400-410, 486-491, 560-570, 633-647, AHF) and Emission filters were used for the acquisition of confocal fluorescence images (BFP (Hoechst 33342) ex.: 405 nm em.: 450/50, GFP (GFP), ex: 488 em: 525/50. Images show brightfield in grey, eGFP channel in green and Hoechst 33342 in blue. Scale bar represents 20 μm. FIG. 9c shows a schematic illustration of the generation of the mCherry-NBD-R₁₀-conjugate. FIG. 9d shows fluorescence imaging of CCL2-cells after incubation with the mCherry double-conjugate. The generation of the mCherry-NBD-R₁₀-conjugate and the fluorescence imaging were carried out as described for FIG. 9a and FIG. 9b.

FIG. 10 shows the reaction of Trastuzumab with diethynyl-phosphinate 1 and subsequent analysis. FIG. 10a shows the general procedure for antibody-rebridging using compound 1. FIG. 10b shows the analysis of Trastuzumab before and after the reaction via SDS-PAGE. FIG. 10c shows the deconvoluted intact protein-MS of the rebridged half-antibody (2× modified with 1) after deglycosylation by PNGaseF.

FIG. 11 shows identification of the two cross-linked cysteine residues of an antibody via cross-linking mass spectrometry. For the MS/MS analysis, rebridged Trastuzumab was deglycosylated using PNGase F followed by tryptic in-gel digest. For the identification of the rebridged cysteine residues a dedicated cross-link search engine (pLink 2, Chen, Z. L.; Meng, J. M.; Cao, Y.; Yin, J. L.; Fang, R. Q.; Fan, S. B.; Liu, C.; Zeng, W. F.; Ding, Y. H.; Tan, D.; Wu, L.; Zhou, W. J.; Chi, H.; Sun, R. X.; Dong, M. Q.; He, S. M. A High-Speed Search Engine PLink 2 with Systematic Evaluation for Proteome-Scale Identification of Cross-Linked Peptides. Nat. Commun. 2019, 10 (1). https://doi.org/10.1038/s41467-019-11337-z) was used.

FIG. 11a shows the only cross-link that could be identified was the cross-link between Trastuzumab light-chain and heavy-chain cysteine. Likely, the rebridged hinge-region could not be detected, because the resulting peptide is relatively large and hydrophobic. FIG. 11b shows that, moreover, the intra-chain cross-link between the two hinge-region cysteins of the heavy-chain was identified.

FIG. 12 shows antibody rebridging using phosphinamidate II. Trastuzumab was rebridged according to the general antibody re-bridging protocol using increasing concentrations of II (see “General procedure for antibody rebridging using phosphinates”). Cross-linking of the antibodies heavy- and light-chain was analysed by SDS-PAGE.

FIG. 13 shows functional modification of Trastuzumab and its biological evaluation. FIG. 13a shows two step modification of the antibody with phosphinate 2, followed by on antibody CuAAC forming the fluorescein conjugate (half- & full-antibody). FIG. 13b shows the analysis of the conjugate via SDS-PAGE using coomassie staining and in-gel fluorescence. FIG. 13c shows a UV-Vis spectrum of the fluorescein conjugated antibody. FIG. 13d shows cell-membrane labelling of Her2-positive cells without any observed staining of Her2-negative cells (Scale bar 20 μm).

FIG. 14 shows antibody labeling using thiovinyl- and triazole-based ethynyl-phosphinates. FIG. 14a shows the reaction scheme. Trastuzumab (5 mg/ml in reaction buffer) is reacted with 8 eq. TCEP and 8 eq. of compounds CS265 and CS266. FIG. 14b shows the structures of CS265 and CS266. FIG. 14c shows analysis of the conjugates by SDS page. FIG. 14d shows analysis of the conjugates by intact-protein MS. A fluorophore to antibody ratio (FAR) of 2.9 with CS265 and 7.4 with CS266 was determined. FIG. 14e shows exemplary deconvoluted intact-protein-MS spectra of other runs of the reaction of Trastuzumab with 1 equivalent or 8 equivalents of phosphinate CS265. FIG. 14f shows exemplary deconvoluted intact-protein-MS spectra of other runs of the reaction of Trastuzumab with 1 equivalent or 8 equivalents of phosphinate CS266. FIG. 14g shows the reaction of non-reduced Trastuzumab with 100 eq. CS266. No labelling of non-reduced Trastuzumab with CS266 could be detected using intact-protein MS. FIG. 14h shows labeling of Trastuzumab with thiovinyl-ethynyl phosphinate CS265, triazolyl-ethynyl phosphinate CS266 and ethynyl-phosphonamidate S1. FIG. 14i shows the titration of Trastuzumab with increasing amounts of phosphinates CS265 and CS266. FIG. 14j shows a time-course experiment, where reduced Trastuzumab was incubated with 10 eq. of the triazolyl-ethynyl phosphinate CS266 and ethynyl phosphonamidate S1.

FIG. 15 shows the determination of the second-order rate constant of the reaction between glutathione and EDANS-phosphinate CS265. FIG. 15a shows the reaction conditions. Reactions were performed in a volume of 0.1 ml. The first sample (t=0) was drawn before the addition of glutathione. Samples were taken after 30, 60, 120, 240 and 1440 min (CS265). Samples were drawn in a volume of 10 μl and immediately diluted into 200 μl of 50 mM NaOAc buffer at pH 3.5, to stop the reaction. Those samples were subjected to fluorescent HPLC analyses, injecting 50 μl each. FIG. 15b shows the mathematic consideration for the determination of a second order rate constant with equal concentrations of the two reactants. FIG. 15c shows the concentration of starting material over time. Calculated by integration of the peaks in relation to the internal standard (EDANS). Shown are mean and error of three independent measurements.(n=3) FIG. 15d shows the graph: 1/c over time and linear plot. Slope is the second order rate constant. Shown are mean and error of three independent measurements.

FIG. 16 shows the determination of the second-order rate constant of the reaction between glutathione and EDANS-phosphinates CS266. FIG. 16a shows the reaction conditions. Reactions were performed in a volume of 0.1 ml. The first sample (t=0) was drawn before the addition of glutathione. Samples were taken after 1, 2, 5, 12, 22 and 35 min (CS266). Samples were drawn in a volume of 10 μl and immediately diluted into 200 μl of 50 mM NaOAc buffer at pH 3.5, to stop the reaction. Those samples were subjected to fluorescent HPLC analyses, injecting 50 μl each. FIG. 16b shows the mathematic consideration for the determination of a second order rate constant with equal concentrations of the two reactants. FIG. 16c shows the concentration of starting material over time. Calculated by integration of the peaks in relation to the internal standard (EDANS). Shown are mean and error of three independent measurements.(n=3) FIG. 16d shows the graph: 1/c over time and linear plot. Slope is the second order rate constant. Shown are mean and error of three independent measurements.

FIG. 17 shows a fluorophore-quencher based assay to investigate the stability of triazole-phosphinate thiol-adduct (FRET-Pair 4 (F4)), and the stability of the antibody fluorescein conjugate Trastuzumab-CS375 in human serum. FIG. 17a shows the structure of the phosphinate linked dye-quencher conjugate and principle of the fluorescence-quencher based readout. FIG. 17b shows the results of the fluorescence measurements for conjugate F4. FIG. 17c shows the analysis of samples obtained after incubating the antibody fluorescein conjugate Trastuzumab-CS375 with human serum after different times of incubation.

FIG. 18 shows the preparation of compound CS298 and rebridging of an antibody using this compound. FIG. 18a shows the preparation of compound CS298 from fluorescein-azide (FAM-N₃) and tri-ethynyl-phosphinoxide. FIG. 18b shows rebridging of an antibody using CS298. SDS-PAGE analysis showed a high degree (>85%) of rebridged antibody.

FIG. 19 shows protein labeling of eGFP with a triazole vinyl phosphinate CS321. FIG. 19a shows the reaction scheme. FIG. 19b shows the structure of compound CS321. FIG. 19c shows the mass spectrum of the labeled eGFP.

FIG. 20 shows mass spectra of protein eGFP C70M S147C.

FIG. 21 shows mass spectra of modified protein eGFP C70M S124C-1.

FIG. 22 shows mass spectra of histone H3-3-1.

FIG. 23 shows mass spectra of recombinant BSA-1.

FIG. 24 shows mass spectra of conjugate eGFP-1-Glutathione.

FIG. 25 shows mass spectra of conjugate eGFP-1-R₁₀.

FIG. 26 shows mass spectra of rebridged antibody Trastuzumab-1.

FIG. 27 shows mass spectra of rebridged antibody Trastuzumab-2.

FIG. 28 shows the click reaction of Trastuzumab-2 with FAM-N₃, analysis of the conjugate via SDS-PAGE using Coomassie staining and in-gel fluorescence, and the UV-Vis spectrum of the fluorescein-conjugated antibody.

FIG. 29 shows NMR spectra of ethyl diethynyl phosphinate (1). FIG. 29a shows the ¹H NMR (600 MHz, DMSO-d₆) spectrum of ethyl diethynyl phosphinate (1). FIG. 29b shows the ³¹P-NMR (122 MHz, CDCl₃) spectrum of ethyl diethynyl phosphinate (1). FIG. 29c shows the ¹³C NMR (75 MHz, CDCl₃) spectrum of ethyl diethynyl phosphinate (1).

FIG. 30 shows NMR spectra of the Z/Z-isomer of ethyl bis(2-(ethylthio)vinyl)phosphinate (5). FIG. 30a shows the ¹H NMR (600 MHz, CDCl₃) spectrum of the Z/Z-isomer of ethyl bis(2-(ethylthio)vinyl)phosphinate (5). FIG. 30b shows the ³¹P-NMR (243 MHz, CDCl₃) spectrum of the Z/Z-isomer of ethyl bis(2-(ethylthio)vinyl)phosphinate (5). FIG. 30c shows the ¹³C NMR (151 MHz, CDCl₃) spectrum of the Z/Z-isomer of ethyl bis(2-(ethylthio)vinyl)phosphinate (5).

FIG. 31 shows NMR spectra of the E/Z isomer of ethyl bis(2-(ethylthio)vinyl)phosphinate (5). FIG. 31a shows the ¹H NMR (600 MHz, CDCl₃) spectrum of the E/Z-isomer of ethyl bis(2-(ethylthio)vinyl)phosphinate (5). FIG. 31b shows the ³¹P. NMR (243 MHz, CDCl₃) spectrum of the E/Z-isomer of ethyl bis(2-(ethylthio)vinyl)phosphinate (5). FIG. 31c shows the ¹³C NMR (151 MHz, CDCl₃) spectrum of the E/Z-isomer of ethyl bis(2-(ethylthio)vinyl)phosphinate (5).

FIG. 32 shows NMR spectra of the E/E isomer of ethyl bis(2-(ethylthio)vinyl)phosphinate (5). FIG. 32a shows the ¹H NMR (600 MHz, CDCl₃) spectrum of the E/E-isomer of ethyl bis(2-(ethylthio)vinyl)phosphinate (5). FIG. 32b shows the ³¹P-NMR (243 MHz, CDCl₃) spectrum of the E/Z-isomer of ethyl bis(2-(ethylthio)vinyl)phosphinate (5). FIG. 32c shows the ¹³C NMR (151 MHz, CDCl₃) spectrum of the E/Z-isomer of ethyl bis(2-(ethylthio)vinyl)phosphinate (5).

FIG. 33 shows NMR spectra of but-3-yn-1-yl-diethynylphosphinate (2). FIG. 33a shows the ¹H NMR (300 MHz, CDCl₃) spectrum of but-3-yn-1-yl diethynylphosphinate (2). FIG. 33b shows the ³¹P-NMR (122 MHz, CDCl₃) spectrum of ethyl but-3-yn-1-yl diethynylphosphinate (2). FIG. 33c shows the ¹³C NMR (75 MHz, CDCl₃) spectrum of but-3-yn-1-yl diethynylphosphinate (2).

FIG. 34 shows NMR spectra of mPEG₄ diethynylphosphinate (3). FIG. 34a shows the ¹H NMR (300 MHz, CDCl₃) spectrum of mPEG₄ diethynylphosphinate (3). FIG. 34b shows the ³¹P-NMR (122 MHz, CDCl₃) spectrum of mPEG₄ diethynylphosphinate (3). FIG. 34c shows the ¹³C NMR (75 MHz, CDCl₃) spectrum of mPEG₄ diethynylphosphinate (3).

FIG. 35 shows NMR spectra of NBD diethynyl-phosphinate (4). FIG. 35a shows the ¹H NMR (600 MHz, DMSO-d₆) spectrum of NBD diethynyl-phosphinate (4). FIG. 35b shows the ³¹P-NMR (243 MHz, DMSO-d₆) spectrum of NBD diethynyl-phosphinate (4). FIG. 35c shows the ¹³C NMR (151 MHz, DMSO-d₆) spectrum of NBD diethynyl-phosphinate (4).

FIG. 36 shows NMR spectra of diethyl diethynylphosphinic amide (II). FIG. 36a shows the ¹H NMR (600 MHz, CDCl₃) spectrum of diethyl diethynylphosphinic amide (II). FIG. 36b shows the ³¹P-NMR (243 MHz, CDCl₃) spectrum of diethyl diethynylphosphinic amide (II). FIG. 36c shows the ¹³C NMR (151 MHz, CDCl₃) spectrum of diethyl diethynylphosphinic amide (II).

FIG. 37 shows NMR spectra of compound EDANS-N₃. FIG. 37a shows the ¹H NMR (600 MHz, DMSO-d₆) spectrum of EDANS-N₃. FIG. 37b shows the ¹³C NMR (151 MHz, DMSO-d₆) spectrum of EDANS-N₃.

FIG. 38 shows an HPLC chromatogram of Peptide 1.

FIG. 39 shows an HPLC chromatogram of FRET-Pair 1 (F1).

FIG. 40 shows an HPLC chromatogram of FRET-Pair 2 (F2).

FIG. 41 shows an HPLC chromatogram of FRET-Pair 3 (F3).

FIG. 42 shows NMR spectra of compound EDANS-1,2,3-triazol-ethyl-ethynylphosphinate (CS266). FIG. 42a shows the ¹H NMR (600 MHz, DMSO-d₆) spectrum of EDANS-1,2,3-triazol-ethyl-ethynylphosphinate (CS266). FIG. 42b shows the ¹³C NMR (151 MHz, DMSO-d₆) spectrum of EDANS-1,2,3-triazol-ethyl-ethynylphosphinate (CS266). FIG. 42c shows the ³¹P NMR (243 MHz, DMSO-d₆) spectrum of EDANS-1,2,3-triazol-ethyl-ethynylphosphinate (CS266).

FIG. 43 shows NMR spectra of compound Biotin-1,2,3-triazol-ethyl-ethynylphosphinate (CS292). FIG. 43a shows the ¹H NMR (600 MHz, DMSO-d₆) spectrum. FIG. 43b shows the ¹³C NMR (151 MHz, DMSO-d₆) spectrum. FIG. 43c shows the ³¹p NMR (243 MHz, DMSO-d₆) spectrum.

FIG. 44 shows NMR spectra of compound diethynyl(phenyl)phosphine oxide (CS267). FIG. 44a shows the ¹H NMR (600 MHz, DMSO-d₆) spectrum. FIG. 44b shows the ³¹P NMR (243 MHz, DMSO-d₆) spectrum. FIG. 44c shows the ¹³C NMR (151 MHz, DMSO-d₆) spectrum.

FIG. 45 shows NMR spectra of compound diethynyl(ethyl)phosphine oxide (CS297-Sideproduct). FIG. 45a shows the ¹H NMR (600 MHz, Chloroform-d) spectrum). FIG. 45b shows the ¹³C NMR (151 MHz, Chloroform-d) spectrum. FIG. 45c shows the ³¹P NMR (243 MHz, Chloroform-d) spectrum.

FIG. 46 shows a ³¹P NMR spectrum of CS298.

FIG. 47 shows an HPLC chromatogram of CS298.

FIG. 48 shows an HPLC chromatogram of CS314.

FIG. 49 shows mass spectra of Mass Spectra of NLS-mCherry-5.

FIG. 50 shows mass spectra of conjugate NLS-mCherry-5-R₁₀.

FIG. 51 shows the synthesis and biological evaluation of the ADC Trastuzumab-CSDrug1. FIG. 51a shows the synthetic route towards the functionalized toxic payload CSDrug1 that is used in the generation of a Trastuzumab based ADC. 1) 0.2 eq. MMAE-VC-PEG₄-N₃, 10 mol % CuBr, PBS/DMSO (2:8, v/v), 4 h r.t., 59% yield. 11) 0.2 eq. Trastuzumab (5 mg/ml), 8 eq. TCEP (with respect to Trastuzumab), Tris-buffer (50 mM, pH 8.3), 1 mM EDTA, 100 mM NaCl. FIG. 51b shows the hydrophilic-interaction-chromatography of the purified ADC (Trastuzumab-CSDrug1; DAR 4.3). FIG. 51c shows the concentration dependent cellular cytotoxicity in Her2+ (SKBR3, green) and Her2− (MDA-MB-468, black) cell lines. FIG. 51d shows the concentration dependent cellular cytotoxicity in Her2+ (SKBR3, left) and Her2− (MDA-MB-468, right), and non-functionalized Trastuzumab as control, obtained from testing the ADC Trastuzumab-CSDrug1 in a proliferation assay. Cells were treated as described in Example 17 below with regard to the cell based anti proliferation assays with SKBR3 and MDA-MB-468. Trastuzumab-CSDrug1 shows dose-dependent toxicity against the Her2+ cell line SKBR3 with an IC50 of 72 pm. In contrast Her2-cells (MDA-MB-468) were not affected by the ADC. The non-functionalized Trastuzumab control did not show any cytotoxicity at the tested concentrations. FIG. 51e shows intact-protein MS of the crude reaction mixture of Trastuzumab-CSDrug1.

FIG. 52 shows the protein-protein conjugation according to Example 14. FIG. 52a shows the synthetic strategy to obtain electrophilic ubiquitin from site-selectively installed K→Aha mutants. FIG. 52b shows an intact protein-MS of the ETP-functionalized ubiquitin 13. The abbreviation “ETP”, when used herein, refers to “ethynyl-triazolyl-phosphinate”, i.e. a phosphinate wherein a triazolyl moiety and an ethynyl moiety are bound to the phosphorus. FIG. 52c shows an intact-protein MS of the artificial Ub-dimer 15. FIG. 52d shows the time course of the conjugation of 12 to UbG76C monitored by SDS-Page. FIG. 52e shows the MS/MS-spectrum identifying the linkage-site of 15. FIG. 52f shows the SDS-PAGE analysis of Ub-dimers 14 & 15 incubated with USP2.

FIG. 53 shows the time course of the UbG76C-UbK63PT dimer (13) formation. UbK63PT (200 μM) was reacted with 2.5 eq. freshly reduced UbG76C as described in Example 17 under ETP-functionalization of UbK48Aha and UbK63Aha. After 0, 2, 4 and 6 hours a sample was drawn and analyzed via intact-protein MS. Deconvoluted mass spectra were normalized to the UbG76C peak, plotted and stacked using Graphpad Prism 5 software.

FIG. 54 shows the MS/MS-analysis of artificial ubiquitin dimers. FIG. 54a shows the MS/MS-analysis of UbK48-ETP-UbG76C. FIG. 54b shows the MS/MS-analysis of UbK63-ETP-UbG76C. For the MS/MS analysis, UbK48-ETP-UbG76C (a) and UbK63-ETP-UbG76C (b) dimers were prepared as described in Example 17 under ETP-funcionalization of UbK48Aha and UbK63Aha and analyzed as described in Example 16 under Proteomics Data Analysis—Ubiquitin dimers using Proteome Discoverer (v. 2.5.0.400) and MS Amanda 2.0. Exemplary spectra show the best scoring peptide-spectrum-match (PSM) identifying the correct linkage site.

FIG. 55 shows the investigation of proteome-wide cysteine reactivity of ETP-electrophiles. FIG. 55a shows the workflow for the labelling of whole-cell lysate using the fluorescent phosphinate CS375 and subsequent analysis. FIG. 55b shows the SDS-PAGE analysis of cell lysate treated with increasing electrophile concentration. FIG. 55c shows the illustration of the workflow used for the unbiased analysis of electrophile selectivity via MS-based proteomics. FIG. 55d shows the histogram of the modifications detected in MS-Fragger open search across three replicates. Δm of phosphinate CS418 is highlighted in green. ox=oxidation (+15.99 Da), f=formylation (+27.99 Da), CAM=carbamidomethylation (+57.02 Da). FIG. 55e shows the abundance of identified modification sites when using Δmₑₓₚ as offset-mass. #of PSMs represents the sum of three replicates.

FIG. 56 shows the proteome wide amino acid selectivity applying a ΔScore >1. The proteome-wide amino acid selectivity was determined as described in Example 16 under Proteomics Data Analysis and Example 17 under Sample preparation for proteome-wide cysteine profiling with an additional ΔScore filter of >1.

FIG. 57 shows the proteome-wide cysteine-profiling. Comparison of the protein input for cysteine proteomics using phosphinate CS418. Data was analyzed using Proteome Discoverer (v. 2.5.0.400) and MS Amanda 2.0. Values shown are the sum of three replicates.

FIG. 58 shows the mass spectra of the conjugate Trastuzumab-CS375 obtained by intact-protein MS. FIG. 58a shows the non-deconvoluted MS-spectrum. FIG. 58b shows the deconvoluted MS-spectrum.

FIG. 59 shows the intact-protein mass spectra of UBK48-ETP and UbK63-ETP. FIG. 59a shows the intact-protein MS of UBK48-EPT. The peak with a mass of 8739.1 Da corresponds to UbK48M, an impurity from auxotrophic expression. FIG. 59b shows the intact-protein MS of UbK63-ETP.

FIG. 60 shows intact-protein MS of the UbK48ETP-UbG76C dimer 12 and the UbK63ETP-UbG76C dimer 13. FIG. 60a shows intact-protein MS of the UbK48ETP-UbG76C dimer 12. FIG. 60b shows intact-protein MS of the UbK63ETP-UbG76C dimer 13.

FIG. 61 shows the SDS-PAGE analysis of dimer 12 incubated with USP2-CD.

FIG. 62 shows NMR spectra of compound ethyl norbornene-PEG₇-triazol-ethynyl-phosphinate (CS390). FIG. 62a shows the ¹H NMR (600 MHz, acetonitrile-d₃) spectrum. FIG. 62b shows the ¹³C NMR (151 MHz, acetonitrile-d₃) spectrum. FIG. 62c shows the ³¹P NMR (234 MHz, acetonitrile-d₃) spectrum.

FIG. 63 shows an HPLC chromatogram of compound 5/6-carboxyfluorescein-1,2,3-triazol-ethyl-ethynylphosphinate (CS375).

FIG. 64 shows NMR spectra of compound desthiobiotin-1,2,3-triazol-ethyl-ethynylphosphinate (CS418). FIG. 64a shows the ¹H NMR (600 MHz, DMSO-d₆) spectrum. FIG. 64b shows the ¹³C NMR (151 MHz, DMSO-d₆) spectrum. FIG. 64c shows the ³¹P NMR (234 MHz, DMSO-d₆) spectrum.

FIG. 65 shows an HPLC chromatogram of compound Cy5-1,2,3-triazol-ethyl-ethynylphosphinate (CS450).

FIG. 66 shows NMR spectra of compound 3-(4-(2-(2-(2-azidoethoxy)ethoxy)ethoxy)phenyl)-6-methyl-1,2,4,5-tetrazine (CS414). FIG. 66a shows the ¹H NMR (600 MHz, acetonitrile-d₃) spectrum. FIG. 66b shows the ¹³C NMR (151 MHz, acetonitrile-d₃) spectrum.

FIG. 67 shows NMR spectra of compound tetrazine-PEG3-triazolyl-phosphinoxide (CS415). FIG. 67a shows the ¹H NMR (600 MHz, DMSO-d₆) spectrum. FIG. 67b shows the ¹³C NMR (151 MHz, DMSO-d₆) spectrum. FIG. 67c shows the ³¹P NMR (234 MHz, DMSO-d₆) spectrum.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. The person skilled in the art is aware that the terms “a” or “an”, as used in the present application, may, depending on the situation, mean “one (1)” “one (1) or more” or “at least one (1)”. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one or more such as one, two, three, four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### Definitions

Halogen, unless defined otherwise: elements of the 7^{th} main group, preferably fluorine, chlorine, bromine and iodine, more preferably fluorine, chlorine and bromine and, in combination with Mg even more preferably bromine.

Alkyl, unless defined otherwise elsewhere: saturated straight-chain or branched hydrocarbon radicals having preferably (C₁-C₈)-, (C₁-C₆)- or (C₁-C₄)-carbon atoms. Examples: methyl, ethyl, propyl, 1-methylethyl, butyl, etc.

Alkenyl, unless defined otherwise elsewhere: unsaturated straight-chain or branched hydrocarbon radicals having a double bond. Alkenyl is preferably (C₂-C₈)-, (C₂-C₆)- or (C₂-C₄)-alkenyl. Examples: ethenyl, 1-propenyl, 3-butenyl, etc.

Alkynyl, unless defined otherwise elsewhere: unsaturated straight-chain or branched hydrocarbon radicals having a triple bond. Alkynyl is preferably (C₂-C₈)-, (C₂-C₆)- or (C₂-C₄)-alkynyl. Examples: ethynyl, 1-propynyl, etc.

Alkoxy (alkyl radical —O—), unless defined otherwise elsewhere: an alkyl radical which is attached via an oxygen atom (—O—) to the basic structure. Alkoxy is preferably (C₁-C₈)-, (C₁-C₆)- or (C₁-C₄)-alkoxy. Examples: methoxy, ethoxy, propoxy, 1-methylethoxy, etc.

Analogously, alkenoxy and alkynoxy, unless defined otherwise elsewhere, are alkenyl radicals and alkynyl radicals, respectively, which are attached via —O— to the basic structure. Alkenoxy is preferably (C₂-C₈)-, (C₆-C₆)- or (C₂-C₄)-alkenoxy. Alkynoxy is preferably (C₃-C₁₀)-, (C₃-C₆)- or (C₃-C₄)-alkynoxy.

Alkylcarbonyl (alkyl radical —C(═O)—), unless defined otherwise: alkylcarbonyl is preferably (C₁-C₈)-, (C₁-C₆)- or (C₁-C₄)-alkylcarbonyl. Here, the number of carbon atoms refers to the alkyl radical in the alkylcarbonyl group.

Analogously, alkenylcarbonyl and alkynylcarbonyl, are, unless defined otherwise elsewhere: alkenyl radicals and alkynyl radicals, respectively, which are attached via —C(═O)— to the basic structure. Alkenylcarbonyl is preferably (C₂-C₈)-, (C₂-C₆)- or (C₂-C₄)-alkenylcarbonyl. Alkynylcarbonyl is preferably (C₂-C₈)-, (C₂-C₆)- or (C₂-C₄)-alkynylcarbonyl.

Alkoxycarbonyl (alkyl radical —O—C(═O)—), unless defined otherwise elsewhere: alkoxycarbonyl is preferably (C₁-C₈)-, (C₁-C₆)- or (C₁-C₄)-alkoxycarbonyl. Here, the number of carbon atoms refers to the alkyl radical in the alkoxycarbonyl group.

Analogously, alkenoxycarbonyl and alkynoxycarbonyl, unless defined otherwise elsewhere, are: alkenyl radicals and alkynyl radicals, respectively, which are attached via —O—C(═O)— to the basic structure. Alkenoxycarbonyl is preferably (C₂-C₈)-, (C₂-C₆)- or (C₂-C₄)-alkenoxycarbonyl. Alkynoxycarbonyl is preferably (C₃-C₈)-, (C₃-C₆)- or (C₃-C₄)-alkynoxycarbonyl.

Alkylcarbonyloxy (alkyl radical —C(═O)—O—), unless defined otherwise elsewhere: an alkyl radical which is attached via a carbonyloxy group (—C(═O)—O—) by the oxygen to the basic structure. alkylcarbonyloxy is preferably (C₁-C₈)-, (C₁-C₆)- or (C₁-C₄)-alkylcarbonyloxy.

Analogously, alkenylcarbonyloxy and alkynylcarbonyloxy, unless defined otherwise elsewhere, are: alkenyl radicals and alkynyl radicals, respectively, which are attached via (—C(═O)—O—) to the basic structure. Alkenylcarbonyloxy is preferably (C₂-C₈)-, (C₂-C₆)- or (C₂-C₄)-alkenylcarbonyloxy. Alkynylcarbonyloxy is preferably (C₂-C₈)-, (C₂-C₆)- or (C₂-C₄)-alkynylcarbonyloxy.

Alkylthio, unless defined otherwise elsewhere: an alkyl radical which is attached via —S— to the basic structure. Alkylthio is preferably (C₁-C₈)-, (C₁-C₆)- or (C₁-C₄)-alkylthio.

Analogously, alkenylthio and alkynylthio, unless defined otherwise elsewhere, are: alkenyl radicals and alkynyl radicals, respectively, which are attached via —S— to the basic structure. Alkenylthio is preferably (C₂-C₈)-, (C₂-C₆)- or (C₂-C₄)-alkenylthio. Alkynylthio is preferably (C₃-C₈)-, (C₃-C₆)- or (C₃-C₄)-alkynylthio.

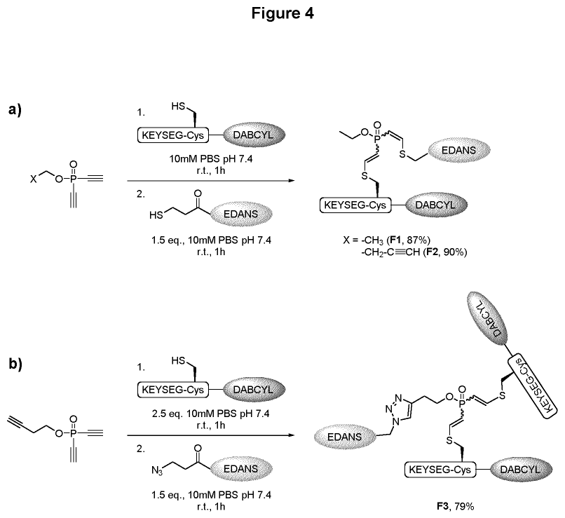

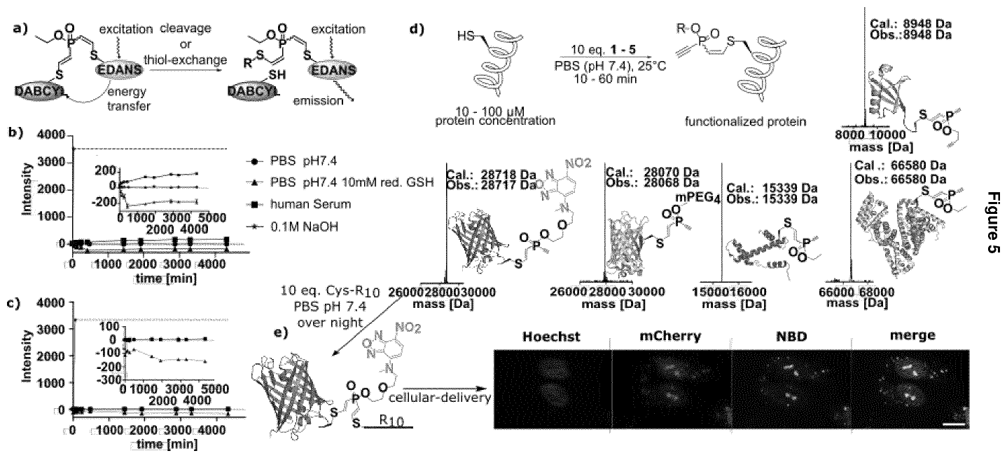

The term “substituted”, or “optionally substituted”, or the like as used unless defined otherwise elsewhere, refers to a very broad substitution pattern. As can be seen from the disclosure of this invention, especially position R₁, Z, [symbol] , ● and ▴, allow the substitution with numerous organic (macro)molecules. It is submitted that the structure of these molecules is not relevant for the presently disclosed process and the resulting conjugates. Thus, it would represent an undue limitation to limit the principle of this new and innovative concept to only some molecules. Nevertheless, it is submitted that the term refers to organic substituents or salts thereof, respectively, which may again be substituted several times by further organic substituents or salts thereof, respectively. Examples for such complex substituents were produced and are presented in this application (see, e.g., Schemes 1b and 1c; FIGS. 4, 5, 6, 9, 13, 14, 15, 16, 17, 18 and 28; and the synthetic examples). Preferably, the term substituted refers to groups which are substituted with one or more substituents selected from nitro, cyano, Cl, F, Cl, Br, —NH—R, NR₂, COOH, —COOR, —OC(O)R—NH₂, —OH, —CONH₂ CONHR, CON(R)₂, —S—R, —SH, —C(O)H, —C(O)R, (C₁-C₂₀)-alkyl, (C₁-C₂₀)-alkoxy, (C₂-C₂₀)-allyl, (hetero)cyclic rings of 3 to 8 ring-members wherein, if present, the heteroatom or atoms are independently selected from N, O and S, (hetero)aromatic systems with 5 to 12 ring atoms (e.g., phenyl, pyridyl, naphtyl etc.), wherein R again can represent any of these substituents and the substitution can be repeated several times, for example, substitution can be repeated for 1, 2, 3, 4, 5, 6, 7, 8, 9 or 10 times; see, e.g. the R₁ substituent in the following:

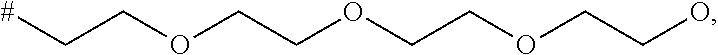

wherein represents the position of Y in the compounds used herein, if R₁ is already part of a compound of e.g. formula (I) or (III). However, the skilled person will agree that an alkyl-chain which is substituted e.g. with a polysaccharide of 40 units, or a protein or an antibody, cannot be simply described by general substitution pattern.

The terms “peptide” as used herein refers to an organic compound comprising two or more amino acids covalently joined by peptide bonds (amide bond). Peptides may be referred to with respect to the number of constituent amino acids, i.e., a dipeptide contains two amino acid residues, a tripeptide contains three, etc. Peptides containing ten or fewer amino acids may be referred to as oligopeptides, while those with more than ten amino acid residues, e.g. with up to about 30 amino acid residues, are polypeptides. The amino acids can form at least one circle or a branched or unbranched chain or mixtures thereof. Proteins and antibodies are peptides and, thus, encompassed by the term, but may be named separately, due to their importance.

The term “amino acid” as used herein refers to an organic compound having a —CH(NH₃)—COOH group. In one embodiment, the term “amino acid” refers to a naturally occurring amino acid. As illustrative examples, naturally occurring amino acids include arginine, lysine, aspartic acid, glutamic acid, glutamine, asparagine, histidine, serine, threonine, tyrosine, cysteine, methionine, tryptophan, alanine, isoleucine, leicine, phenylalanine, valine, proline and glycine. However, the term in its broader meaning also encompasses non-naturally occurring amino acids.

Amino acids and peptides according to the invention can also be modified at functional groups. Non limiting examples are saccharides, e.g., N-Acetylgalactosamine (GaINAc), or protecting groups, e.g., Fluorenylmethoxycarbonyl (Fmoc)-modifications or esters.

The term “protein” refers to peptides which comprise one or more long chains of amino acid residues, e.g. with more than about 30 amino acid residues. Proteins perform a vast array of functions in vivo and in vitro including catalysing metabolic reactions, DNA replication, responding to stimuli, and transporting molecules, catalysing reactions. Proteins are folded into a specific three-dimensional structure. The residues in a protein are often chemically modified, e.g., by post-translational modification, which alters the physical and chemical properties, folding, stability, activity, and ultimately, the function of the proteins. Sometimes proteins have non-peptide groups attached, which can be called prosthetic groups or cofactors. Proteins, including enzymes and coenzymes, can also work together to achieve a particular function, and they often associate to form stable protein complexes. All these forms are encompassed by the term “protein”.

The term “protein tags” as used herein refers to peptide sequences which can be attached to proteins or other thiol-comprising compounds via a linker according to the present invention for various purposes. Non limiting examples for protein tags are affinity tags, solubilization tags, chromatography tags epitope tags and reporter enzymes.

Affinity tags are appended to proteins and other thiol-comprising compounds via the linker according to the present invention so that they can be, e.g., purified using an affinity technique. These include for example chitin binding protein (CBP), maltose binding protein (MBP), and glutathione-S-transferase (GST) or the poly(His) tag.

Solubilization tags can be used to assist in the proper folding in proteins and keep them from precipitating. These include thioredoxin (TRX) and poly(NANP). Some affinity tags have a dual role as a solubilization agent, such as MBP, and GST.

Chromatography tags are used to alter chromatographic properties of the protein to afford different resolution across a particular separation technique. Often, these consist of polyanionic amino acids, such as FLAG-tag.

Epitope tags are short peptide sequences which are chosen because high-affinity antibodies can be reliably produced in many different species. These are usually derived from viral genes. Epitope tags include V5-tag, Myc-tag, HA-tag and NE-tag. These tags are particularly useful for western blotting, immunofluorescence and immunoprecipitation experiments, and antibody purification.

The term “reporter enzymes” as used herein refer to any known enzyme which allows an increase of a signal in a biochemical detection. Non limiting examples are, colorant forming enzymes such as alkaline phosphatase (AP), horseradish peroxidase (HRP) or glucose oxidase (GOX); fluorescent proteins, such as green fluorescence protein (GFP), redox sensitive GFP (RoGFP), Azurite or Emerald; luciferase, i.e. a class of oxidative enzymes that produce bioluminescence (e.g. firefly luciferase (EC 1.13.12.7)); chloramphenicol acetyl transferase (CAT); ß-galactosidase; or ß-glucuronidase.

Non-limiting examples of protein tags are: AviTag, a peptide allowing biotinylation by the enzyme BirA and so the protein can be isolated by streptavidin (GLNDIFEAQKIEWHE), Calmodulin-tag, a peptide bound by the protein calmodulin (KRRWKKNFIAVSAANRFKKISSSGAL), polyglutamate tag, a peptide binding efficiently to anion-exchange resin such as Mono-Q (EEEEEE), E-tag, a peptide recognized by an antibody (GAPVPYPDPLEPR), FLAG-tag, a peptide recognized by an antibody (DYKDDDDK), HA-tag, a peptide from hemagglutinin recognized by an antibody (YPYDVPDYA)His-tag, 5-10 histidines bound by a nickel or cobalt chelate (HHHHHH), Myc-tag, a peptide derived from c-myc recognized by an antibody (EQKLISEEDL), NE-tag, a novel 18-amino-acid synthetic peptide (TKENPRSNQEESYDDNES) recognized by a monoclonal IgG1 antibody, which is useful in a wide spectrum of applications including Western blotting, ELISA, flow cytometry, immunocytochemistry, immunoprecipitation, and affinity purification of recombinant proteins, S-tag, a peptide derived from Ribonuclease A (KETAAAKFERQHMDS), SBP-tag, a peptide which binds to streptavidin (MDEKTTGWRGGHVVEGLAGELEQLRARLEHHPQGQREP), Softag 1, for mammalian expression (SLAELLNAGLGGS), Softag 3, for prokaryotic expression (TQDPSRVG), Strep-tag, a peptide which binds to streptavidin or the modified streptavidin called streptactin (Strep-tag II: WSHPQFEK), TC tag, a tetracysteine tag that is recognized by FlAsH and ReAsH biarsenical compounds (CCPGCC), V5 tag, a peptide recognized by an antibody (GKPIPNPLLGLDST), VSV-tag, a peptide recognized by an antibody (YTDIEMNRLGK), Xpress tag (DLYDDDDK), Isopeptag, a peptide which binds covalently to pilin-C protein (TDKDMTITFTNKKDAE), SpyTag, a peptide which binds covalently to SpyCatcher protein (AHIVMVDAYKPTK),SnoopTag, a peptide which binds covalently to SnoopCatcher protein (KLGDIEFIKVNK), BCCP (Biotin Carboxyl Carrier Protein), a protein domain biotinylated by BirA enabling recognition by streptavidin, Glutathione-S-transferase-tag, a protein which binds to immobilized glutathione, Green fluorescent protein-tag, a protein which is spontaneously fluorescent and can be bound by nanobodies, Halo-tag, a mutated hydrolase that covalently attaches to the HaloLink™ Resin (Promega), Maltose binding protein-tag, a protein which binds to amylose agarose, Nus-tag, Thioredoxin-tag, Fc-tag, derived from immunoglobulin Fc domain, allow dimerization and solubilization. Can be used for purification on Protein-A Sepharose, Designed Intrinsically Disordered tags containing disorder promoting amino acids (P,E,S,T,A,Q,G, . . . ), alkaline phosphatase (AP), horseradish peroxidase (HRP) glucose oxidase (GOX), green fluorescence protein (GFP), redox sensitive GFP (RoGFP), Azurite, Emerald, firefly luciferase (EC 1.13.12.7)), chloramphenicol acetyl transferase (CAT), β-galactosidase, β-glucuronidase, tubulin-tyrosine ligase (TTL).

The term “antibody”, as used herein, is intended to refer to immunoglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs arranged from amino-terminus to carboxy-terminus e.g. in the following order FR1, CDR1, FR2, CDR2, FR3, CDR3, FR4. The term “antibody” or “antibody molecule”, as used herein, includes an intact full length antibody and an antigen-binding fragment of an antibody.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H3) in the heavy chain variable domain; and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA1, and IgA2. A preferred class of immunoglobulins for use in the present invention is IgG.

The heavy-chain constant domains that correspond to the different classes of antibodies are called [alpha], [delta], [epsilon], [gamma], and [mu], respectively. The subunit structures and three-dimensional configurations of different classes of immunoglobulins are well known. As used herein antibodies are conventionally known antibodies and functional fragments thereof.

A “functional fragment”, or “antigen-binding antibody fragment” of an antibody/immunoglobulin, or “antigen-binding fragment of an antibody”, or an “antibody fragment”, or a “fragment of an antibody” hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). In many instances, the present disclosure refers to an “antibody molecule”. The term “antibody molecule”, as used herein, also includes “a functional fragment”, an “antigen-binding antibody fragment”, “an antigen-binding fragment of an antibody”, an “antibody fragment”, or a “fragment of an antibody”, or the like.

“Functional fragments”, “antigen-binding antibody fragments”, “antigen-binding fragments of an antibody”, or “antibody fragments” or “fragments of an antibody” of the invention include, but are not limited to, those which contain at least one disulfide bond that can be reacted with a reducing agent as described herein. Examples of suitable fragments include Fab, Fab′, Fab′-SH, F(ab′)₂, a “half antibody molecule” as used herein which consists of one antibody heavy chain and one antibody light chain, Fv fragments; diabodies; single domain antibodies (DAbs), linear antibodies; single-chain antibody molecules (scFv); and multispecific, such as bi- and tri-specific, antibodies formed from antibody fragments. An antibody other than a “multi-specific” or “multi-functional” antibody is understood to have each of its binding sites identical. The F(ab′)₂ or Fab may be engineered to minimize or completely remove the intermolecular disulfide interactions that occur between the CH1 and CL domains.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index.

Variants of the antibodies or antigen-binding antibody fragments contemplated in the invention are molecules in which the binding activity of the antibody or antigen-binding antibody fragment is maintained.

“Binding proteins” contemplated in the invention are for example antibody mimetics, such as Affibodies, Adnectins, Anticalins, DARPins, Avimers, Nanobodies.

A “human” antibody or antigen-binding fragment thereof is hereby defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source).

A “humanized antibody” or humanized antigen-binding fragment thereof is defined herein as one that is (i) derived from a non-human source (e.g., a transgenic mouse which bears a heterologous immune system), which antibody is based on a human germline sequence; (ii) where amino acids of the framework regions of a non-human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is defined herein as one, wherein the variable domains are derived from a non-human origin and some or all constant domains are derived from a human origin.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or nonproteinaceous solutes.

As used herein, an antibody “binds specifically to”, is “specific to/for” or “specifically recognizes” an antigen of interest, e.g. a tumor-associated polypeptide antigen target, is one that binds the antigen with sufficient affinity such that the antibody is useful as a therapeutic agent in targeting a cell or tissue expressing the antigen, and does not significantly cross-react with other proteins or does not significantly cross-react with proteins other than orthologs and variants (e.g. mutant forms, splice variants, or proteolytically truncated forms) of the aforementioned antigen target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to surface plasmon resonance (SPR), Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” or “affinity” refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K₀ value” according to this invention is measured by using surface plasmon resonance assays using suitable devices including but not limited to Biacore instruments like Biacore T100, Biacore T200, Biacore 2000, Biacore 4000, a Biacore 3000 (GE Healthcare Biacore, Inc.), or a ProteOn XPR36 instrument (Bio-Rad Laboratories, Inc.).

The terms “nucleoside” and “nucleoside moiety” as use herein reference a nucleic acid subunit including a sugar group and a heterocyclic base, as well as analogs of such sub-units, such as a modified or naturally occurring deoxyribonucleoside or ribonucleoside or any chemical modifications thereof. Other groups (e.g., protecting groups) can be attached to any component(s) of a nucleoside. Modifications of the nucleosides include, but are not limited to, 2′-, 3′- and 5′-position sugar modifications, 5- and 6-position pyrimidine modifications, 2-, 6- and 8-position purine modifications, modifications at exocyclic amines, substitution of 5-bromo-uracil, and the like. Nucleosides can be suitably protected and derivatized to enable oligonucleotide synthesis by methods known in the field, such as solid phase automated synthesis using nucleoside phosphoramidite monomers, H-phosphonate coupling or phosphate triester coupling.

A “nucleotide” or “nucleotide moiety” refers to a sub-unit of a nucleic acid which includes a phosphate group, a sugar group and a heterocyclic base, as well as analogs of such sub-units. Other groups (e.g., protecting groups) can be attached to any component(s) of a nucleotide. The term “nucleotide”, may refer to a modified or naturally occurring deoxyribonucleotide or ribonucleotide. Nucleotides in some cases include purines and pyrimidines, which include thymidine, cytidine, guanosine, adenine and uridine. The term “nucleotide” is intended to include those moieties that contain not only the known purine and pyrimidine bases, e.g. adenine (A), thymine (T), cytosine (C), guanine (G), or uracil (U), but also other heterocyclic bases that have been modified. Such modifications include methylated purines or pyrimidines, acylated purines or pyrimidines, alkylated riboses or other heterocycles. Such modifications include, e.g., diaminopurine and its derivatives, inosine and its derivatives, alkylated purines or pyrimidines, acylated purines or pyrimidines thiolated purines or pyrimidines, and the like, or the addition of a protecting group such as acetyl, difluoroacetyl, trifluoroacetyl, isobutyryl, benzoyl, 9-fluorenylmethoxycarbonyl, phenoxyacetyl, dimethylformamidine, dibutylformamidine, dimethylacetamidine, N,N-diphenyl carbamate, or the like. The purine or pyrimidine base may also be an analog of the foregoing; suitable analogs will be known to those skilled in the art and are described in the pertinent texts and literature. Common analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine.

The term “oligonucleotide”, as used herein, refers to a polynucleotide formed from a plurality of linked nucleotide units as defined above. The nucleotide units each include a nucleoside unit linked together via a phosphate linking group, or an analog thereof. The term oligonucleotide also refers to a plurality of nucleotides that are linked together via linkages other than phosphate linkages such as phosphorothioate linkages or squaramide linkages. The oligonucleotide may be naturally occurring or non-naturally occurring. In some cases, the oligonucleotides may include ribonucleotide monomers (i.e., may be oligoribonucleotides) and/or deoxyribonucleotide monomers. As illustrative examples, the oligonucleotides may comprise of from 2 to 50 nucleotide units, e.g. of from 2 to 40 nucleotide units, e.g. of from 5 to 35 nucleotide units, e.g. of from 10 to 35 nucleotide units, e.g of from 15 to 30 nucleotide units.

The term “monosaccharide” as use herein refers to an open chained or cyclic compound of general formula Cₘ(H₂O)ₙ wherein m is 3, 4, 5, 6, 7 or 8 and n is 2, 3, 4, 5 6, 7 or 8. However, the term also encompasses derivatives of these basic compounds wherein a OH group is replaced by an NH₂ group (such as glucosamine), desoxysaccharides, wherein at least one OH group is replaced by H (e.g. desoxiribose). Preferred examples for monosaccharides are D-(+)-Glycerinaldehyd; D-(−)-Erythrose; D-(−)-Threose; D-(−)-Ribose; D-(−)-Arabinose; D-(+)-Xylose; D-(−)-Lyxose; D-(+)-Allose; D-(+)-Altrose; D-(+)-Glucose; D-(+)-Mannose; D-(−)-Gulose; D-(−)-Idose; D-(+)-Galactose; D-(+)-Talose; Dihydroxyaceton; D-Erythrulose; D-Ribulose; D-Xylulose; D-Psicose; D-Fructose; D-Sorbose; D-Tagatose. The term monosaccharide also encompasses monosaccharides which one, two, three or four hydroxyl-groups are substituted.

The term “polysaccharides” refers to molecules comprising at least 2 (two), preferably at least 5 (five), more preferably at least 10 (ten) monosaccharides which are connected via a glycosidic bond.

A carbohydrate as used herein encompasses a monosaccharide and a polysaccharide and derivatives thereof.

A polymer as used herein refers to macromolecules composed of many repeated organic subunits, however, which are no polysaccharides, oligonucleotides or peptides. Examples for polymers are Polyethylenglycole (PEG), polyoxyethylene (PEO) or polyglycerol (e.g. polyglycerol-polyricinoleate (PGPR).

The term “fluorophore” is well-known to the skilled person and refers to chemical compounds that re-emit light upon light excitation. Non limiting examples are CY₅, EDANS, Xanthene derivatives (e.g. fluorescein, Rhodamine, Oregon green, eosin, Texas red), Cyanine derivatives (e.g., indocarbocyanine, oxacarbocyanine, merocyanine), Squaraine derivatives (e.g., Seta, Se Tau, Square dyes), Naphthalene derivatives (e.g., dansyl or prodan derivatives), Coumarin derivatives, Oxadiazole derivatives, Anthracene derivatives (e.g., Anthraquinones such as DRAQ5, DRAQ7, CyTRAK Orange), Pyrene derivatives (e.g., cascade blue), Oxazine derivatives (e.g., Nile red, Nile blue, Cresyl violet), Acridine derivatives (e.g., Proflavin, Acridine Orange, Acridine Yellow), Arylmethine derivatives (e.g., Auramine, Crystal Violet, Malachite Green), or Tetrapyrrole derivatives (e.g., Parphin, Phthal ocyanine, Bilirubin).

The term “aliphatic or aromatic residue” as used herein refers to an aliphatic substituent, e.g. an alkyl residue which, however, can be optionally substituted by further aliphatic and/or aromatic substituents, e.g. an aliphatic residue can be a nucleic acid, a peptide, a protein, an enzyme, a co-enzyme, an antibody, a nucleotide, an oligonucleotide, a monosaccharide, a polysaccharide, a polymer, a fluorophore, optionally substituted benzene, etc. as long as the direct link of such a molecule to the core structure (in case of R₁, e.g., to the respective Y, e.g. oxygen, of a compound of e.g. formula (I), (III), (IIIa), (IV) or (IV*), or of a conjugate of an antibody molecule as described herein) is aliphatic. An aromatic residue is a substitute, wherein the direct link to the core structure is part of an aromatic system, e.g., an optionally substituted phenyl or triazolyl or pyridyl or peptide, e.g., if the direct link of the peptide to the core structure is for example via a phenyl-residue. The term “aromatic residue”, as used herein, also includes a heteroaromatic residue.

The term “antibody drug conjugate” or abbreviated ADC is well known to a person skilled in the art, and, as used herein, refers to the linkage of an antibody or an antigen binding fragment thereof with a drug, such as a chemotherapeutic agent, a toxin, an immunotherapeutic agent, an imaging probe, and the like. As used herein, a “linker” is any chemical moiety that links an antibody or an antigen binding fragment thereof covalently to the drug. The linker may be any linker known to a person skilled in the art. As used herein, the term “linker drug conjugate” refers to a molecule or chemical group comprising or consisting of a linker as defined herein before, and a drug. In this regard, the term “linker drug conjugate” in general refers to that part of an antibody drug conjugate which is not the antibody or an antigen binding fragment thereof. In general, in a linker drug conjugate the linker is covalently linked to the drug.

The term “antibody fluorophore conjugates” or abbreviated AFC is also well-known to a person skilled in the art and refers to the linkage of an antibody or an antigen binding fragment thereof with a fluorophore, such as, for example, Cy5. The fluorophore may be linked to the antibody or antigen-binding fragment thereof through a linker. The linker may be any linker known to a person skilled in the art. The antibody fluorophore conjugate may comprise a “linker fluorophore conjugate”. As used herein, the term “linker fluorophore conjugate” refers to a molecule or chemical group comprising or consisting of a linker as defined herein before, and a fluorophore. In this regard, the term “linker fluorophore conjugate” in general refers to that part of an antibody fluorophore conjugate which is not the antibody or an antigen binding fragment thereof. In general, in a linker fluorophore conjugate the linker is covalently linked to the fluorophore.

The term “small molecule” as used herein denotes an organic molecule comprising at least two carbon atoms, but preferably not more than 7, 12, 15 or 20 rotatable carbon bonds, more preferably not more than 7, 12 or 15 rotatable carbon bonds, even more preferably not more than 7 or 12 rotatable carbon bonds, having a molecular weight in the range between 100 and 2000 Dalton, preferably between 100 and 1000 Dalton, and optionally including one or two metal atoms. As merely illustrative examples for small molecules biotin and the fluorophores EDANS and Cy5 may be mentioned.

### Methods

### Method of Preparing a Compound of Formula (III)

The present invention, in one aspect, relates to a method of preparing a compound of formula (III) comprising a step of:
reacting a compound of formula (I)

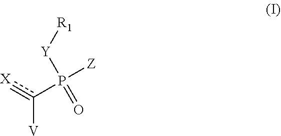

wherein
[symbol] represents a triple bond or a double bond;
V is absent when [symbol] is a triple bond; or
V represents H or C₁-C₈-alkyl when [symbol] is a double bond;
X represents R₃—C when [symbol] is a triple bond; or
X represents

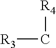

when [symbol] is a double bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl; and
Z represents a residue bound to the phosphorus via a carbon atom and comprising a group ●, wherein ▴ represents an optionally substituted aliphatic or optionally substituted aromatic residue;
with a thiol-containing molecule of formula (II)
wherein [symbol] represents an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system;
resulting in a compound of formula (III)

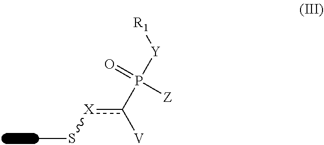

wherein
[symbol] represents a double bond when [symbol] in a compound of formula (I) represents a triple bond; or
[symbol] represents a bond when [symbol] in a compound of formula (I) represents a double bond;
V is absent when [symbol] is a double bond; or
V is H or C₁-C₈-alkyl when [symbol] is a bond;
X represents R₃—C when [symbol] is a double bond; or
X represents

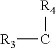

when [symbol] is a bond; and
[symbol] R₁, R₃, R₄, Y and Z are as defined for the compounds of formula (I) and formula (II).

The [symbol] , V, X, Y, R₁, R², R₃, R₄, Z, ●, [symbol] and [symbol] may be as defined herein for any one of the methods, compounds and/or conjugates. Any [symbol] , V, X, Y, R₁, R², R₃, R₄, Z, ●, [symbol] and [symbol] as defined herein for any one of the methods, compounds and/or conjugates may be combined with each other.

The present invention provides a method of reacting thiol-comprising compounds with unsaturated phosphorus(V) compounds. The methods described herein allow to combine a huge amount of different organic residues in positions R₁, Z, ● and [symbol] . As merely illustrative examples, the processes according to the invention are suitable for forming conjugates when [symbol] is an amino acid, a peptide, a protein, an antibody, a nucleotide or an oligonucleotide. As an advantage, thiol groups present in such amino acid, peptide, protein or antibody, such as e.g. a thiol group of a cysteine residue, or thiol groups present in a nucleotide or an oligonucleotide, react chemoselectively with a triple bond or double bond of the unsaturated phosphorus(V) compound, thus providing a chemoselective modification method. Due to such chemoselectivity, the thiol containing compound, as illustrative examples the amino acid, peptide, protein, antibody, nucleotide or oligonucleotide may be unprotected, which means that protecting groups are not necessary. Further, the methods according to the invention allow for conjugation of two complex molecules (e.g. a fluorophore and a protein or an antibody). The obtained conjugates are highly stable, in particular under physiologically relevant conditions, such as for example, in human serum, in the presence of small thiols, and under conditions inside of a living cell. The conjugation works under a broad variety of reaction conditions, for example, under physiologically relevant conditions, such as e.g. physiological pH.

In this context it is noted that compounds falling under the definition of formula (I), namely diethynyl-phosphinates, i.e. molecules having two ethynyl-groups on the same phosphorus atom, have been previously reported in the generation of P-stereogenic heterocycles (J. S. Harvey, G. T. Giuffredi, V. Gouvemeur, Org. Left. 2010, 12, 1236-1239), as intermediates in the synthesis of phospha-scorpionate complexes (S. G. A. Van Assema, C. G. J. Tazelaar, G. De Bas Jong, J. H. Van Maarseveen, M. Schakel, M. Lutz, A. L. Spek, J. Chris Slootweg, K. Lammertsma, Organometallics 2008, 27, 3210-3215) and as building-blocks for macrocycles (S. G. A. van Assema, G. B. de Jong, A. W. Ehlers, F. J. J. de Kanter, M. Schakel, A. L. Spek, M. Lutz, K. Lammertsma, European J. Org. Chem. 2007, 2007, 2405-2412). It has been found in the present invention that these reagents can be used for preparing compounds of formula (III) and conjugates of an antibody molecule, as described herein.

Turning now to the invention in more detail, preferably, in any one of the methods of preparing a compound of formula (III), [symbol] represents a triple bond; V is absent; X represents R₃—C, R₃ represents H or C₁-C₈-alkyl; and [symbol] represents a double bond. Preferably, R₃ represents H or C₁-C₆-alkyl, more preferably H or C₁-C₄-alkyl, still more preferably H or C₁-C₂-alkyl. Even more preferably, R₃ is H.

In some embodiments, in any one of the methods of preparing a compound of formula (III), [symbol] may represent a double bond; V may be H or C₁-C₈-alkyl; X may represent

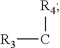

R₃ and R₄ may independently represent H or C₁-C₈-alkyl; [symbol] and may represents a bond. Preferably, R₃ and R₄ independently represent H or C₁-C₆-alkyl, more preferably H or C₁-C₄-alkyl, still more preferably H or C₁-C₂-alkyl. Preferably, R₃ and R₄ are the same; even more preferably, R₃, R₄ and V are the same. More preferably, R₃ and R₄ are both H. Preferably, V is H or C₁-C₆-alkyl, more preferably H or C₁-C₄-alkyl, still more preferably H or C₁-C₂-alkyl. Even more preferably, V is H. In preferred embodiments, R₃, R₄ and V are each H.

With regard to the representations [symbol] and [symbol] used herein, it is noted that, as commonly known to a person skilled in the art, each carbon atom is tetravalent. Accordingly, a structure

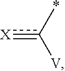

wherein X and V are as defined herein and the asterisk (*) indicates attachment to the phosphorus, includes the structure

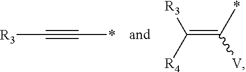

wherein R₃, R₄ and V are as defined herein. A structure

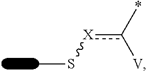

wherein [symbol] X and V are as defined herein and the asterisk (*) indicates attachment to the phosphorus, includes the structures

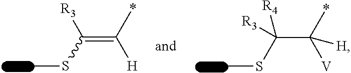

wherein [symbol] , R₃, R₄ and V are as defined herein, and H is hydrogen. A wavy bond indicates that the configuration of the double bond may be E or Z. It is also possible that the compound is present as a mixture of the E and Z isomers.

Preferably, in any one of the methods of preparing a compound of formula (III), Z is

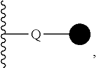

wherein [symbol] indicates the attachment point to the phosphorus and ● is as defined herein; and Q is a moiety comprising at least three main-chain carbon atoms and a carbon-carbon double bond, wherein at least one of the main chain atoms is a heteroatom selected from the group consisting of S, O or N, preferably S. Optionally, in each instance, a linker can be arranged between ● and Q. More preferably, Z is

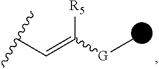

wherein Q is

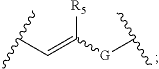

R₅ is H or C₁-C₈-alkyl; G is S, O or NR₁₀, wherein R₁₀ is H or C₁-C₈-alkyl; and ● is as defined herein; optionally, a linker can be arranged between ● and Q. Preferably, R₅ is H or C₁-C₆-alkyl, more preferably R₅ is H or C₁-C₄-alkyl, still more preferably R₅ is H or C₁-C₂-alkyl. Even more preferably, R₅ is H. Preferably, in any one of the methods, when [symbol] is a triple bond and X is R₃—C, R₃ and R₅ are the same; more preferably, when [symbol] is a triple bond and X is R₃—C, R₃ and R₅ are both H. Preferably, in any one of the methods, when [symbol] is a double bond and X is

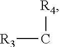

R₃, R₄ and R₅ are the same; more preferably, R₃, R₄, R₅ and V are the same. More preferably, when [symbol] is a double bond and X is

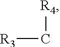

R₃, R₄ and R₅ are each H; even more preferably, R₃, R₄, R₅ and V are H. Preferably, R₁₀, when present, is H or C₁-C₆-alkyl, more preferably H or C₁-C₄-alkyl, still more preferably H or C₁-C₂alkyl. Still more preferably, R₁₀ is H. G may be NR₁₀. G may be O. Preferably, G is S. Accordingly, preferably, Z is

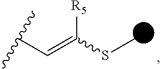

wherein Q is

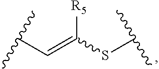

and R₅ and ● are as defined herein; optionally, a linker can be arranged between ● and Q. The method of preparing a compound of formula (III) may further comprise a preparation of a compound of formula (I), said preparation comprising:
reacting a compound of formula (IV)

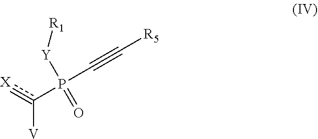

wherein R₁, R₅, [symbol] , V, X and Y are as defined herein, with ●-GH to form a compound of formula (I), wherein G and ● are as defined herein; H is hydrogen; preferably G is S. Optionally, in each instance, a linker can be arranged between ● and G, wherein G is the moiety that will be converted into Q by the reaction with the compound of formula (IV).

Preferably, in any one of the methods of preparing a compound of formula (III), Z is

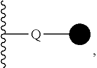

wherein [symbol] indicates the attachment point to the phosphorus and ● is as defined herein; and Q is a five- or six-membered heterocyclic moiety comprising 1, 2 or 3 heteroatoms independently selected from the group consisting of N, O or S. Optionally, in each instance, a linker is arranged between ● and Q. More preferably, Z is selected from the group consisting of

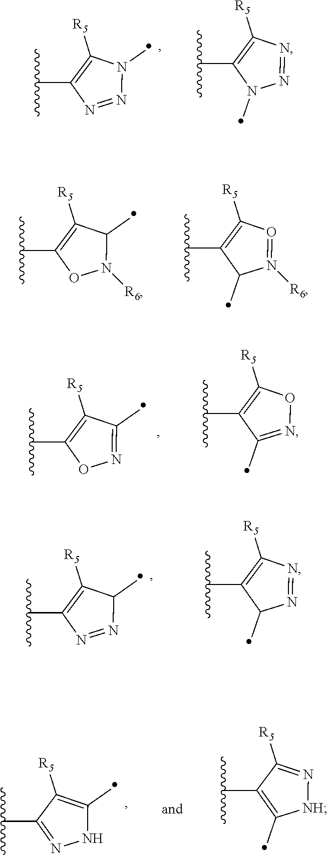

wherein R₅ is H or C₁-C₈-alkyl; R₆ is C₁-C₈-alkyl, and ● is as defined herein. Accordingly, Z may be

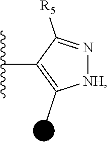

wherein Q is

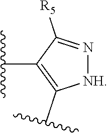

### Z may be

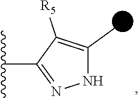

wherein Q is

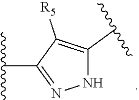

### Z may be

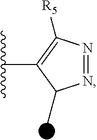

wherein Q is

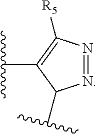

### Z may be

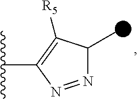

wherein Q is

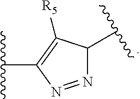

### Z may be

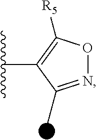

wherein Q is

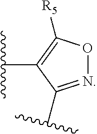

### Z may be

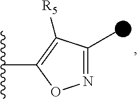

wherein Q is

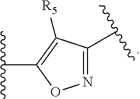

### Z may be

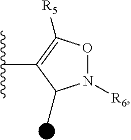

wherein Q is

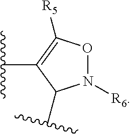

### Z may be

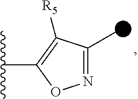

wherein Q is

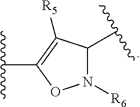

### Preferably, Z is

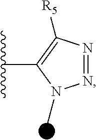

wherein Q is

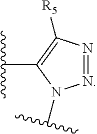

More preferably, Z is

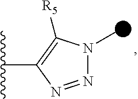

wherein Q is

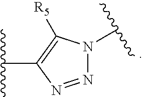

Optionally, in any one of these embodiments a linker can be arranged between ● and Q. Preferably, R₅ is H or C₁-C₆-alkyl, more preferably H or C₁-C₄-alkyl, still more preferably H or C₁-C₂alkyl. Even more preferably, R₅ is H. Preferably, in any one of the methods, when [symbol] is a triple bond and X is R₃—C, R₃ and R₅ are the same; more preferably, when [symbol] is a triple bond and X is R₃—C, R₃ and R₅ are both H. Preferably, in any one of the methods, when [symbol] is a double bond and X is

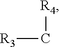

R₃, R₄ and R₅ are the same; even more preferably, R₃, R₄, R₅ and V are the same. More preferably, when [symbol] is a double bond and X is

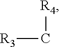

R₃, R₄ and R₅ are each H; more preferably, R₃, R₄, R₅ and V are H. Re, when present, may be C₁-C₈-alkyl, preferably C₁-C₆-alkyl, more preferably C₁-C₄-alkyl, still more preferably C₁-C₂alkyl. The method of preparing a compound of formula (III) may further comprise a preparation of a compound of formula (I), said preparation comprising:
reacting a compound of formula (IV)

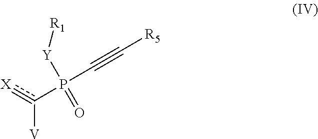

wherein R₁, R₅, [symbol] , V, X and Y are as defined herein,
with ●-N₃,

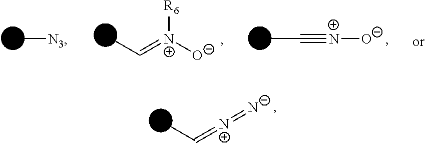

to form a compound of formula (I), wherein ● is as defined herein; and R₆ is C₁-C₈-alkyl, preferably C₁-C₆-alkyl, more preferably C₁-C₄-alkyl, still more preferably C₁-C₂-alkyl. Accordingly the compound of formula (IV) may be reacted with

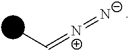

The compound of formula (IV) may be reacted with

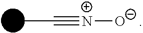

The compound of formula (IV) may be reacted with

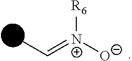

Preferably, the compound of formula (IV) may be reacted with ●-N₃. Preferably, the reacting is carried out in the presence of a catalyst, e.g. a copper catalyst or a ruthenium catalyst. Using a catalyst, e.g. a copper catalyst or a ruthenium catalyst, is particularly preferred when the reacting is carried out with ●-N₃. Optionally, a linker can be arranged between ● and the moiety that will be converted into Q by the reaction with the compound of formula (IV).

Preferably, in any one of the methods of preparing a compound of formula (III), Z is

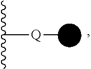

wherein [symbol] indicates the attachment point to the phosphorus and ● is as defined herein; and
Q is a moiety comprising a carbon-carbon triple bond bound to the phosphorus in the compound of formula (I), and an optionally substituted phenyl group bound to the carbon-carbon triple bond; or
Q is a moiety comprising a carbon-carbon triple bond bound to the phosphorus in formula (I), and an optionally substituted carbon-carbon double bond bound to the carbon-carbon triple bond. Optionally, in each instance a linker is arranged between ● and Q. More preferably, Z is

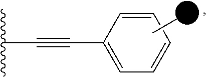

wherein Q is

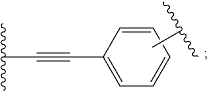

optionally, a linker can be arranged between ● and Q. More preferably, Z is

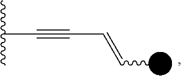

wherein Q is

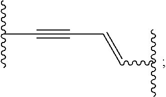

optionally, a linker can be arranged between ● and Q. The method of preparing a compound of formula (III) may further comprise a preparation of the compound of formula (I), said preparation comprising:
reacting a compound of formula (IV)

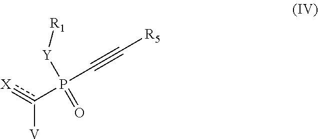

wherein R₁, [symbol] , V, X and Y are as defined herein and R₅ is H, with

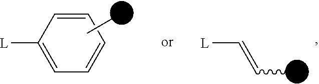

wherein L is halogen (I, Br, Cl, preferably I or Br, more preferably I) or O-triflate to form a compound of formula (I). Preferably, the reacting is carried out in presence of a palladium catalyst, a copper catalyst and a base. For example, the reacting may be carried out as Sonogashira coupling. Optionally, a linker can be arranged between ● and the moiety that will be converted into Q by the reaction with the compound of formula (IV).

Throughout this specification, wherever it is indicated herein with regard to any method, compound or conjugate, that optionally a linker is arranged between ● and Q, or the like, the linker may be virtually any linker known to a person skilled in the art, for example, a peptidic linker or a straight or branched hydrocarbon-based moiety. The linker can also comprise cyclic moieties. A peptidic linker may comprise, for example, 1 to 50, 1 to 40, 1 to 30, 1 to 20, 1 to 10, 1 to 5, 1 to 3, or 2, or 1 amino acid(s). If the linker is a hydrocarbon-based moiety, the main chain of the linker may comprise only carbon atoms but can also contain heteroatoms such as oxygen (O), nitrogen (N) or sulfur (S) atoms, and/or can contain carbonyl groups (C═O). The linker may be, for example, a C₁-C₂₀ carbon atom chain or a polyether-based chain such as a polyethylene glycol-based chain with —(O—CH₂—CH₂)— repeating units. In typical embodiments of hydrocarbon-based linkers, the linking moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. Illustrative example compounds, in which a linker is arranged between ● and Q, are shown in the following:

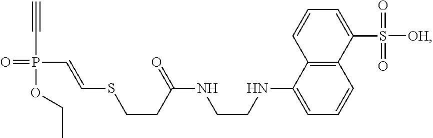 [SMILES: C#CP(=O)(/C=C/SCCC(=O)NCCNc1cccc2c(S(=O)(=O)O)cccc12)OCC]

wherein the linker is

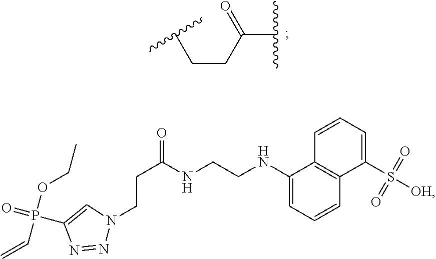 [SMILES: *CCC(*)=O ; C=CP(=O)(OCC)c1cn(CCC(=O)NCCNc2cccc3c(S(=O)(=O)O)cccc23)nn1]

wherein the linker is

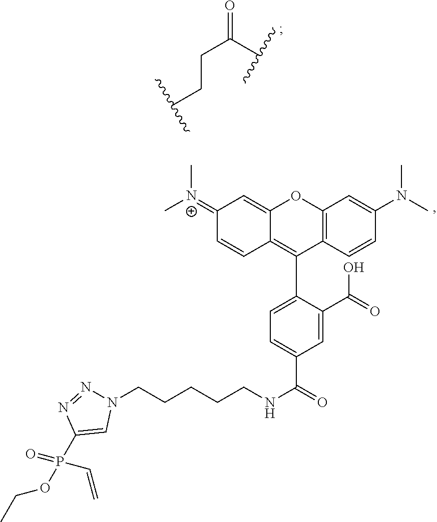 [SMILES: *CCC(*)=O ; C=CP(=O)(OCC)c1cn(CCCCCNC(=O)c2ccc(-c3c4ccc(=[N+](C)C)cc-4oc4cc(N(C)C)ccc34)c(C(=O)O)c2)nn1]

wherein the linker is

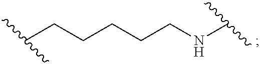 [SMILES: *CCCCCN*]

and

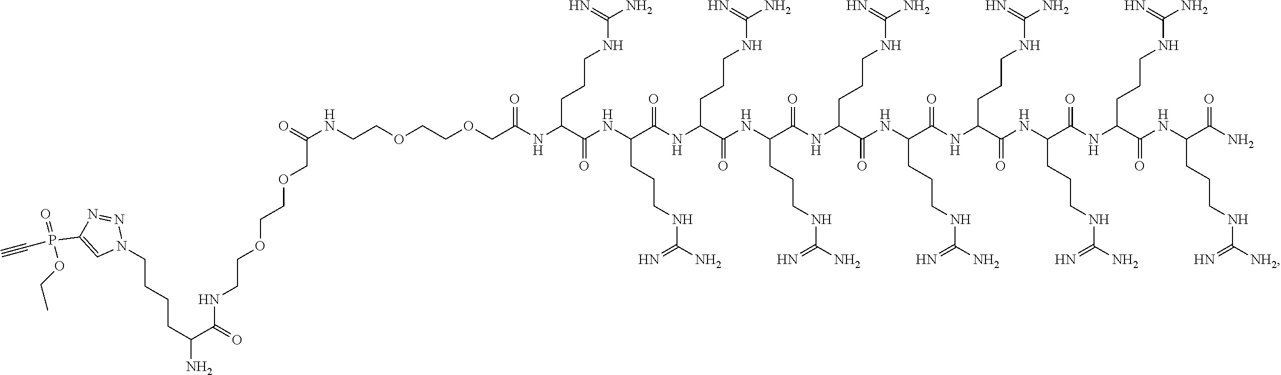 [SMILES: C#CP(=O)(OCC)c1cn(CCCCC(N)C(=O)NCCOCCOCC(=O)NCCOCCOCC(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O)nn1]

wherein the linker is

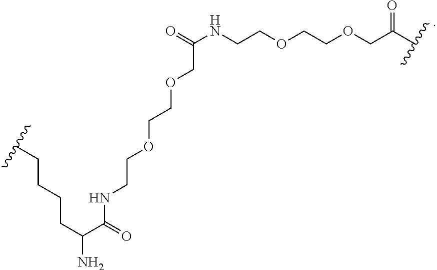 [SMILES: *CCCCC(N)C(=O)NCCOCCOCC(=O)NCCOCCOCC(*)=O]

The aforementioned exemplary linkers may be also used, for example, when the present specification refers to a “linker” as such, or to a “linker-drug conjugate”, for example in the context of an antibody drug conjugate, or to a “linker-fluorophore conjugate”, for example in the context of an antibody fluorophore conjugate. A person skilled in the art knows to select suitable linkers.

In any one of the methods of preparing a compound of formula (III), Y represents O, NR² wherein R² represents H or C₁-C₈-alkyl, S, or a bond. Preferably, in each instance, R₁ is bound to Y via a carbon atom.

Accordingly, Y may be O (oxygen).
Y may be NR². R² is H or C₁-C₈-alkyl. Preferably, R² is C₁-C₈-alkyl. More preferably, R² is methyl, ethyl, propyl or butyl. Still more preferably, R² is methyl or ethyl.

Y may be S (sulfur).

Y may be a bond. In particular, Y may be a single bond which connects R₁ with the phosphorus.

R₁, whenever mentioned throughout the present specification, may be any aliphatic or aromatic residue, which can be optionally substituted, and which does not interfere with the method of preparing a compound of formula (III) as described herein. Accordingly, R₁ covers a broad spectrum of aliphatic or aromatic residues, such as e.g. a group adjusting the water-solubility (e.g. an ethyleneglycol oligomer), a group which can be used for further functionalization (e.g. a group comprising a carbon-carbon triple bond which can be further functionalized, e.g., a so-called “click-handle” which can be further functionalized by a 1,3-dipolar cycloaddition), or a fluorophore (see, e.g., below Examples 2 and 7). A person skilled in the art knows to select suitable residues R₁ which are compatible with the methods described herein.

In any one of the methods of preparing a compound of formula (III), R₁ ma represent a small molecule; C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; preferably in any one of these embodiments Y is O. Accordingly, R₁ may be a small molecule. R₁ may be C₁-C₈-alkyl optionally substituted with (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with at least one of F, Cl, Br, I, —NO₂, —N(C₁-C₈-alkyl)H, —NH₂, —N₃, —N(C₁-C₈-alkyl)₂, ═O, C₃-C₈-cycloalkyl, and/or —S—S—(C₁-C₈-alkyl). R₁ may be C₁-C₈-alkyl optionally substituted with hydroxy-(C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with C₂-C₈-alkenyl. R₁ may be C₁-C₈-alkyl optionally substituted with C₂-C₈-alkynyl. Preferably, in any one of these embodiments Y is O.

In any one of the methods of preparing a compound of formula (III), R₁ may represent phenyl optionally independently substituted with at least one of C₁-C₈-alkyl, (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; preferably in each instance Y is a bond. Accordingly, R₁ may be phenyl optionally substituted with C₁-C₈-alkyl. R₁ may be phenyl optionally substituted with(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be phenyl optionally substituted with at least one of F, Cl, I, Br, —NO₂, —N(C₁-C₈-alkyl)H, —NH₂, and/or —N(C₁-C₈-alkyl)₂. R₁ may be phenyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably, in any one of these embodiments Y is a bond.

In any one of the methods of preparing a compound of formula (III), R₁ may represent a 5- or 6-membered heteroaromatic system such as optionally substituted triazolyl or optionally substituted pyridyl. Preferably, in any one of these embodiments Y is a bond.

In any one of the methods of preparing a compound of formula (III), R₁ may represent a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; or C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore. Accordingly, R₁ may represent a small molecule, and preferably Y may be O. R₁ may represent C₁-C₈-alkyl, and preferably Y may be O. R₁ may represent C₁-C₈-alkyl substituted with —S—S—(C₁-C₈-alkyl), and preferably Y may be O. R₁ may represent C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₁-C₈-alkyl substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₂-C₈-alkenyl, and preferably Y may be O. R₁ may represent C₁-C₈-alkyl substituted with optionally substituted phenyl, and preferably Y may be O. R₁ may represent C₂-C₈-alkynyl, and preferably Y may be O. R₁ may represent phenyl, and preferably Y may be a bond. R₁ may represent phenyl substituted with —NO₂, and preferably Y may be a bond. R₁ may represent triazolyl substituted with optionally substituted C₁-C₈-alkyl, and preferably Y may be a bond. R₁ may represent triazolyl substituted with a fluorophore, and preferably Y may be a bond.

Preferably, in any one of the methods of preparing a compound of formula (III), R₁ may represent C₁-C₈-alkyl. Preferably, R₁ represents methyl, ethyl, propyl or butyl. More preferably, R₁ represents methyl or ethyl. Still more preferably, R₁ represents ethyl. Preferably, in any one of these embodiments R₁ is O.

In any one of the methods of preparing a compound of formula (III), R₁ may be selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O. Accordingly, R₁ may be a small molecule. R₁ may be a fluorophore. R₁ may be optionally substituted C₁-C₈-alkyl, preferably methyl, ethyl, propyl or butyl, more preferably methyl or ethyl, still more preferably ethyl. R₁ may be optionally substituted C₂-C₈-alkenyl. R₁ may be optionally substituted optionally substituted C₂-C₈-alkinyl. Preferably, in any one of these embodiments Y is O.

Preferably, in any one of the methods of preparing a compound of formula (III), R₁ is selected from the group consisting of ethyl; C₁-C₈-alkyl optionally substituted with (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

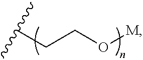

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

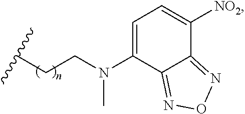

wherein n is 1, 2, 3, 4, 5, 6, 7, 8, 9 or 10, more preferably 4, 5 or 6, still more preferably 5, or more preferably R₁ is

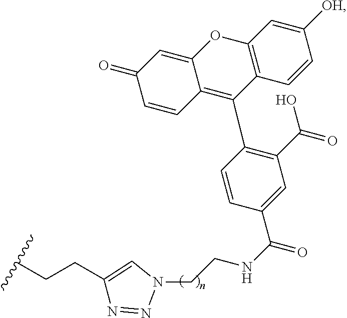

wherein n is 1, 2, 3, 4, 5, 6, 7, 8, 9 or 10, preferably 3, 4 or 5, still more preferably 4; C₂-C₈-alkynyl, preferably

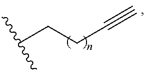

wherein n is 1, 2, 3, 4, or 5, preferably 1, 2 or 3, more preferably 1; or preferably R₁ is

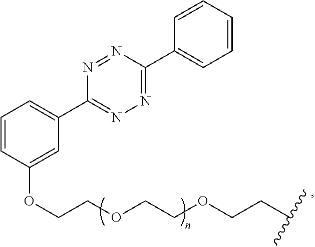

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

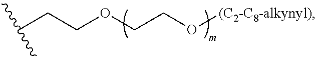

more preferably

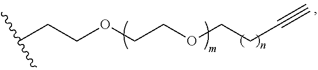

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O. Accordingly, R₁ may be ethyl. R₁ may be C₁-C₈alkyl optionally substituted with (C₁-C₈alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably R₁ is

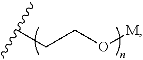

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4. R₁ may be a fluorophore. R₁ may be C₁-C₈-alkyl optionally substituted with a fluorophore. Preferably, R₁ is

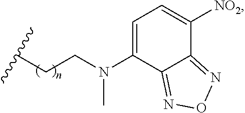

wherein n is 1, 2, 3, 4, 5, 6, 7, 8, 9 or 10, more preferably 4, 5 or 6, still more preferably 5. Preferably, R₁ is

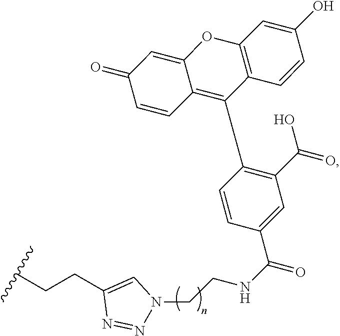

wherein n is 1, 2, 3, 4, 5, 6, 7, 8, 9 or 10, preferably 3, 4 or 5, still more preferably 4. R₁ may be C₂-C₈-alkynyl. Preferably, R₁ is

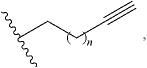

wherein n is 1, 2, 3, 4, or 5, preferably 1, 2 or 3, more preferably 1. Preferably, R₁ is

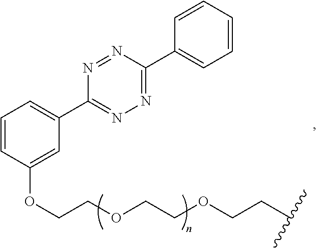

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2. Preferably, R₁ is

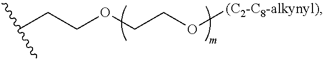

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3. More preferably, R₁ is

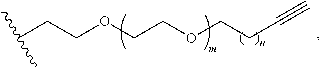

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1. Preferably, in any one of these embodiments Y is O.

In any one of the methods of preparing a compound of formula (III), R₁ may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

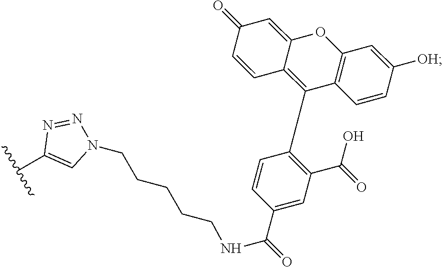 [SMILES: *c1cn(CCCCCNC(=O)c2ccc(-c3c4ccc(=O)cc-4oc4cc(O)ccc34)c(C(=O)O)c2)nn1]

or still more preferably R₁ is

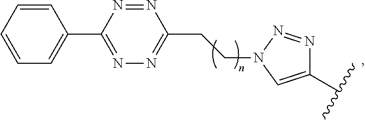

wherein n is 1, 2, 3, 4, 5, 6, 7, 8 or 9, preferably 1, 2 or 3, more preferably 1; or preferably R₁ is

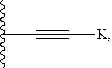

wherein K is H or C₁-C₈-alkyl, preferably K is H; preferably wherein in each instance Y is a bond. Accordingly, R₁ may be optionally substituted aryl. Preferably, R₁ is optionally substituted phenyl. More preferably, R₁ is unsubstituted phenyl. R₁ may be optionally substituted heteroaryl. Preferably, R₁ is optionally substituted triazolyl. More preferably, R₁ is triazolyl substituted with optionally substituted C₁-C₈-alkyl. R₁ may be a fluorophore. More preferably, R₁ is triazolyl substituted with a fluorophore. Still more preferably, R₁ is

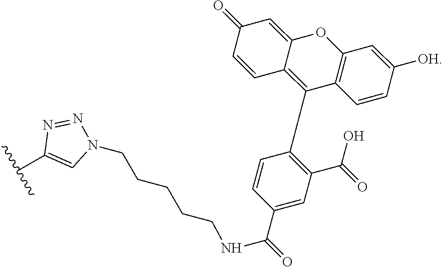 [SMILES: *c1cn(CCCCCNC(=O)c2ccc(-c3c4ccc(=O)cc-4oc4cc(O)ccc34)c(C(=O)O)c2)nn1]

Still more preferably, R₁ is

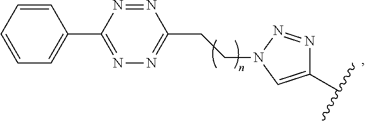

wherein n is 1, 2, 3, 4, 5, 6, 7, 8 or 9, preferably 1, 2 or 3, more preferably 1. Preferably R₁ is

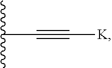

wherein K is H or C₁-C₈-alkyl, preferably H or C₁-C₆-alkyl, more preferably H or C₁-C₄-alkyl, sill more preferably H or C₁-C₈-alkyl; even more preferably K is H. Preferably, in any one of these embodiments Y is a bond.

In any one of the methods of preparing a compound of formula (III), R₁ may be C₁-C₈-alkyl, preferably methyl, ethyl, propyl or butyl; more preferably methyl or ethyl; still more preferably ethyl; and Z may be

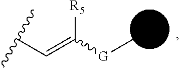

wherein Q is

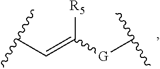

R₅ is as defined herein, preferably R₅ is H; G is S, O or NR₁₀, wherein R₁₀ is as defined herein, preferably R₁₀ is H; and ● is as defined herein. Preferably, Z is

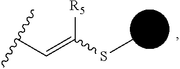

wherein Q is

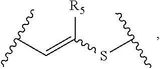

R₅ is as defined herein, preferably R₅ is H; and ● is as defined herein. Preferably, in any one of these embodiments Y is O. Optionally, in any one of these embodiments, R₁ may be C₁-C₈-alkyl substituted with a fluorophore. Optionally, in any one of these embodiments, a linker may be arranged between ● and Q.

In any one of the methods of preparing a compound of formula (III), R₁ may be C₂-C₈-alkynyl, preferably

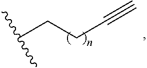

wherein n is 1, 2, 3, 4, or 5, preferably 1, 2 or 3, more preferably 1; and Z may be

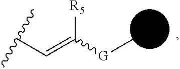

wherein Q is

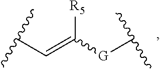

R₅ is as defined herein, preferably R₅ is H; G is S, O or NR₁₀, wherein R₁₀ is as defined herein, preferably R₁₀ is H; and ● is as defined herein; preferably, Z is S

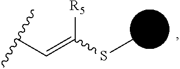

wherein Q is

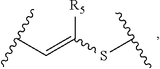

R₅ is as defined herein, preferably R₅ is H, and ● is as defined herein. Preferably, in any one of these embodiments Y is O. Optionally, in any one of these embodiments, a linker may be arranged between ● and Q.

In any one of the methods of preparing a compound of formula (III), R₁ may be

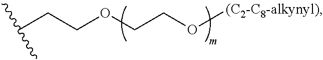

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3; preferably R₁ may be

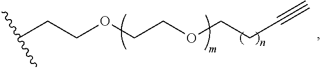

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and Z may be

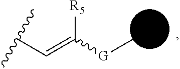

wherein Q is

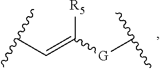

R₅ is as defined herein, preferably R₅ is H; G is S, O or NR₁₀, wherein R₁₀ is as defined herein, preferably R₁₀ is H; and ● is as defined herein. Preferably, Z is

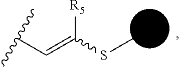

wherein Q is

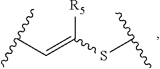

R₅ is as defined herein, preferably R₅ is H, and ● is as defined herein. Preferably, in any one of these embodiments Y is O. Optionally, in any one of these embodiments, a linker may be arranged between ● and Q.

In any one of the methods of preparing a compound of formula (III), R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be

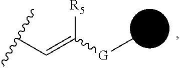

wherein Q is

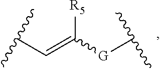

R₅ is as defined herein, preferably R₅ is H; G is S, O or NR₁₀, wherein R₁₀ is as defined herein, preferably R₁₀ is H; and ● is as defined herein. Preferably, Z is

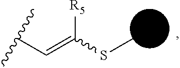

wherein Q is

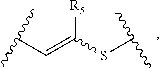

R₅ is as defined herein, preferably R₅ is H, and ● is as defined herein. Preferably, in any one of these embodiments Y is O. Optionally, in any one of these embodiments, a linker may be arranged between ● and Q.

In any one of the methods of preparing a compound of formula (III), R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be

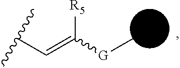

wherein Q is

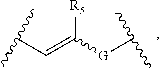

R₅ is as defined herein, preferably R₅ is H; G is S, O or NR₁₀, wherein R₁₀ is as defined herein, preferably R₁₀ is H; and ● is as defined herein. Preferably, Z is

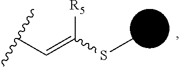

wherein Q is

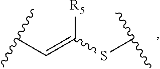

R₅ is as defined herein, preferably R₅ is H, and ● is as defined herein. Preferably, in any one of these embodiments Y is O. Optionally, in any one of these embodiments, a linker may be arranged between ● and Q.

Preferably, in any one of the methods of preparing a compound of formula (III), R₁ is

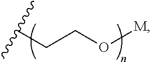

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; and Z may be

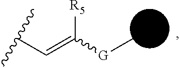

wherein Q is

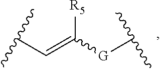

R₅ is as defined herein, preferably R₅ is H; G is S, O or NR₁₀, wherein R₁₀ is as defined herein, preferably R₁₀ is H; and ● is as defined herein; preferably, Z is

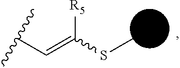

wherein Q is

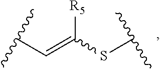

R₅ is as defined herein, preferably R₅ is H, and ● is as defined herein. Preferably, in any one of these embodiments Y is O. Optionally, in any one of these embodiments, a linker may be arranged between ● and Q.

In any one of the methods of preparing a compound of formula (III), R₁ may be C₁-C₈-alkyl, preferably methyl, ethyl, propyl or butyl; more preferably methyl or ethyl; still more preferably ethyl; and Z may be selected from the group consisting of

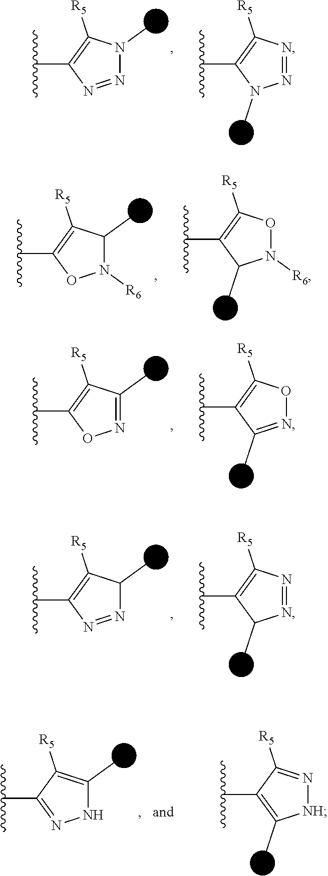

wherein R₅ is as defined herein, preferably R₅ is H; R₆ is as defined herein; and ● is as defined herein. More preferably, Z is

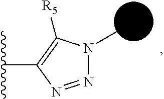

wherein Q is

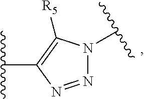

R₅ is as defined herein, preferably R₅ is H; and ● is as defined herein. Preferably, Z is

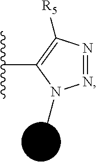

wherein Q is

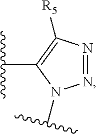

R₅ is as defined herein, preferably R₅ is H; and ● is as defined herein. Z may be

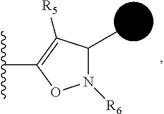

wherein Q is

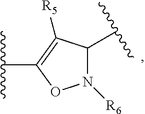

R₅ is as defined herein, preferably R₅ is H; R₆ is as defined herein; and ● is as defined herein. Z may be

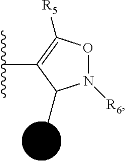

wherein Q is

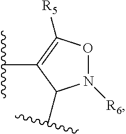

R₅ is as defined herein, preferably R₅ is H; R₆ is as defined herein; and ● is as defined herein. Z may be

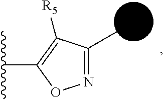

wherein Q is

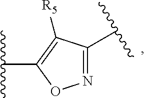

R₅ is as defined herein, preferably R₅ is H; and ● is as defined herein. Z may be

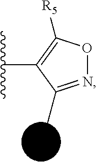

wherein Q is

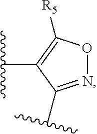

R₅ is as defined herein, preferably R₅ is H; and ● is as defined herein. Z may be

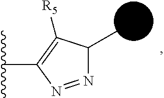

wherein Q is

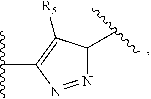

R₅ is as defined herein, preferably R₅ is H; and ● is as defined herein. Z may be

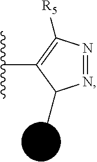

wherein Q is

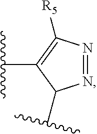

R₅ is as defined herein, preferably R₅ is H; and ● is as defined herein. Z may be

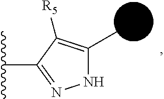

wherein Q is

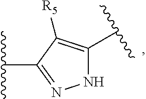

R₅ is as defined herein, preferably R₅ is H; and ● is as defined herein. Z may be

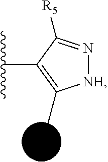

wherein Q is

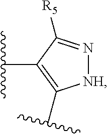

R₅ is as defined herein, preferably R₅ is H; and ● is as defined herein. Preferably, in any one of these embodiments Y is O. Optionally, in any one of these embodiments, R₁ may be C₁-C₈-alkyl substituted with a fluorophore. Optionally, in any one of these embodiments, a linker may be arranged between ● and Q.

In any one of the methods of preparing a compound of formula (III), R₁ may be C₂-C₈-alkynyl, preferably

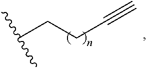

wherein n is 1, 2, 3, 4, 5, 6, 7, 8, 9 or 10, preferably 1, 2 or 3, more preferably 1; and Z may be selected from the group consisting of

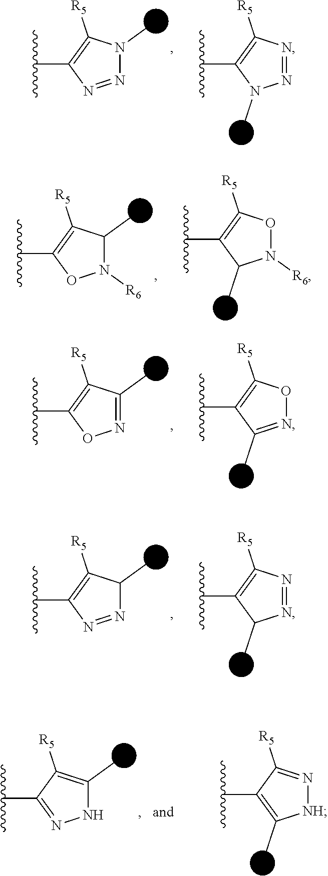

wherein R₅ is as defined herein, preferably R₅ is H; R₆ is as defined herein; and ● is as defined herein. More preferably, Z is

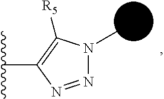

wherein Q is

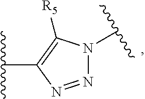

R₅ is as defined herein, preferably R₅ is H; and ● is as defined herein. Preferably, Z is

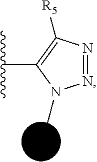

wherein Q is

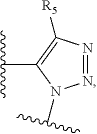

R₅ is as defined herein, preferably R₅ is H; and ● is as defined herein. Z may be

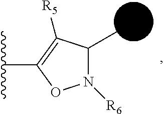

wherein Q is

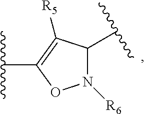

R₅ is as defined herein, preferably R₅ is H; R₆ is as defined herein; and ● is as defined herein. Z may be

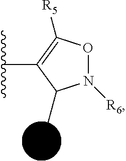

wherein Q is

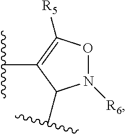

R₅ is as defined herein, preferably R₅ is H; R₆ is as defined herein; and ● is as defined herein. Z may be

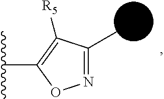

wherein Q is

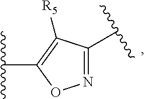

R₅ is as defined herein, preferably R₅ is H; and ● is as defined herein. Z may be

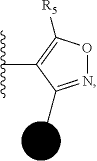

wherein Q is

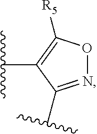

R₅ is as defined herein, preferably R₅ is H; and ● is as defined herein. Z may be

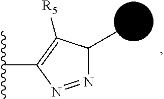

wherein Q is

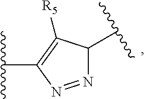

R₅ is as defined herein, preferably R₅ is H; and ● is as defined herein. Z may be

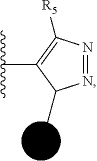

wherein Q is

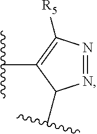

R₅ is as defined herein, preferably R₅ is H; and ● is as defined herein. Z may be

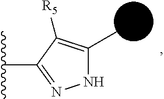

wherein Q is

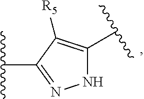

R₅ is as defined herein, preferably R₅ is H; and ● is as defined herein. Z may be

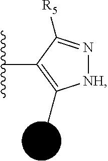

wherein Q is

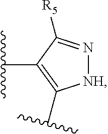

R₅ is as defined herein, preferably R₅ is H; and ● is as defined herein. Preferably, in any one of these embodiments Y is O. Optionally, in any one of these embodiments, a linker may be arranged between ● and Q.

In any one of the methods of preparing a compound of formula (III), R₁ may be

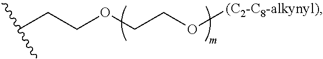

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3; preferably R₁ may be

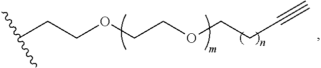

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and Z may be selected from the group consisting of

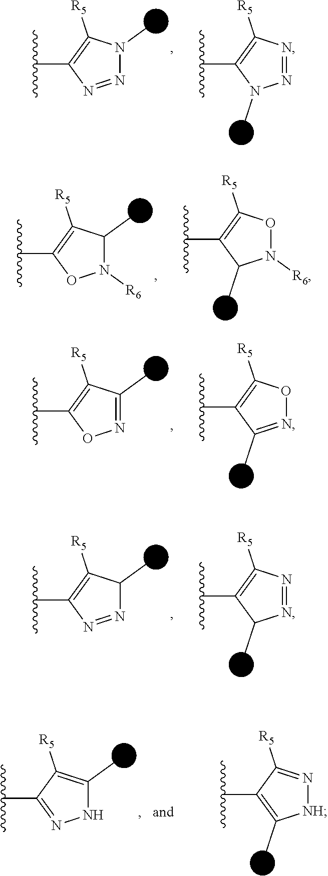

wherein R₅ is as defined herein, preferably R₅ is H; R₆ is as defined herein; and ● is as defined herein. More preferably, Z is

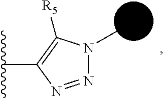

wherein Q is

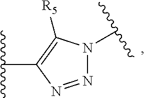

R₅ is as defined herein, preferably R₅ is H; and ● is as defined herein. Preferably, Z is

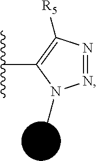

wherein Q is

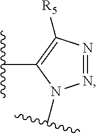

R₅ is as defined herein, preferably R₅ is H; and ● is as defined herein. Z may be

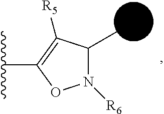

wherein Q is

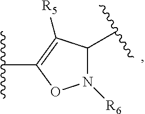

R₅ is as defined herein, preferably R₅ is H; R₆ is as defined herein; and ● is as defined herein. Z may be

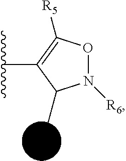

wherein Q is

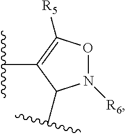

R₅ is as defined herein, preferably R₅ is H; R₆ is as defined herein; and ● is as defined herein. Z may be

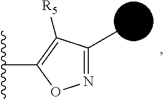

wherein Q is

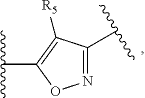

R₅ is as defined herein, preferably R₅ is H; and ● is as defined herein. Z may be

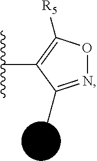

wherein Q is

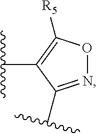

R₅ is as defined herein, preferably R₅ is H; and ● is as defined herein. Z may be

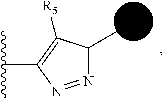

wherein Q is

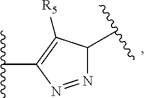

R₅ is as defined herein, preferably R₅ is H; and ● is as defined herein. Z may be

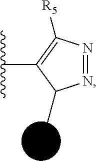

wherein Q is

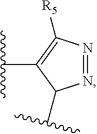

R₅ is as defined herein, preferably R₅ is H; and ● is as defined herein. Z may be

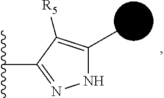

wherein Q is

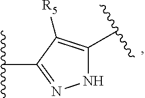

R₅ is as defined herein, preferably R₅ is H; and ● is as defined herein. Z may be

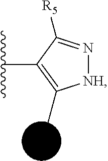

wherein Q is

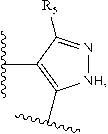

R₅ is as defined herein, preferably R₅ is H; and ● is as defined herein. Preferably, in any one of these embodiments Y is O. Optionally, in any one of these embodiments, a linker may be arranged between ● and Q.

In any one of the methods of preparing a compound of formula (III), R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be selected from the group consisting of

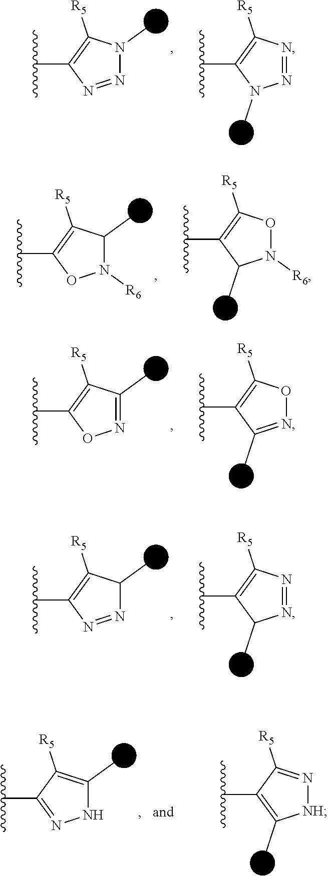

wherein R₅ is as defined herein, preferably R₅ is H; R₆ is as defined herein; and ● is as defined herein. More preferably, Z is

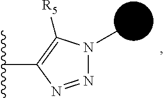

wherein Q is

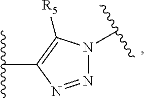

R₅ is as defined herein, preferably R₅ is H; and ● is as defined herein. Preferably, Z is

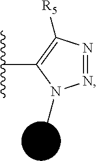

wherein Q is

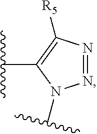

R₅ is as defined herein, preferably R₅ is H; and ● is as defined herein. Z may be

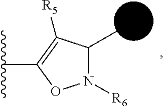

wherein Q is

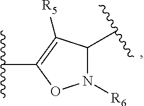

R₅ is as defined herein, preferably R₅ is H; R₆ is as defined herein; and ● is as defined herein. Z may be

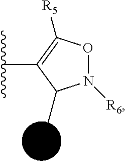

wherein Q is

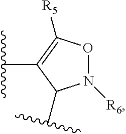

R₅ is as defined herein, preferably R₅ is H; R₆ is as defined herein; and ● is as defined herein. Z may be

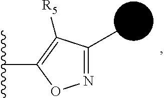

wherein Q is

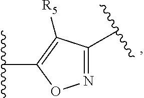

R₅ is as defined herein, preferably R₅ is H; and ● is as defined herein. Z may be

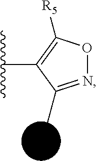

wherein Q is

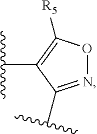

R₅ is as defined herein, preferably R₅ is H; and ● is as defined herein. Z may be

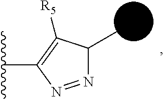

wherein Q is

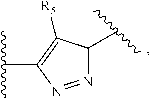

R₅ is as defined herein, preferably R₅ is H; and ● is as defined herein. Z may be

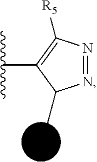

wherein Q is

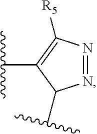

R₅ is as defined herein, preferably R₅ is H; and ● is as defined herein. Z may be

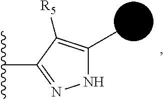

wherein Q is

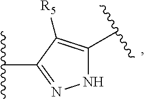

R₅ is as defined herein, preferably R₅ is H; and ● is as defined herein. Z may be

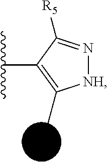

wherein Q is

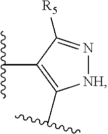

R₅ is as defined herein, preferably R₅ is H; and ● is as defined herein. Optionally, in any one of these embodiments Y is O. Optionally, in any one of these embodiments, a linker may be arranged between ● and Q.

In any one of the methods of preparing a compound of formula (III), R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be selected from the group consisting of

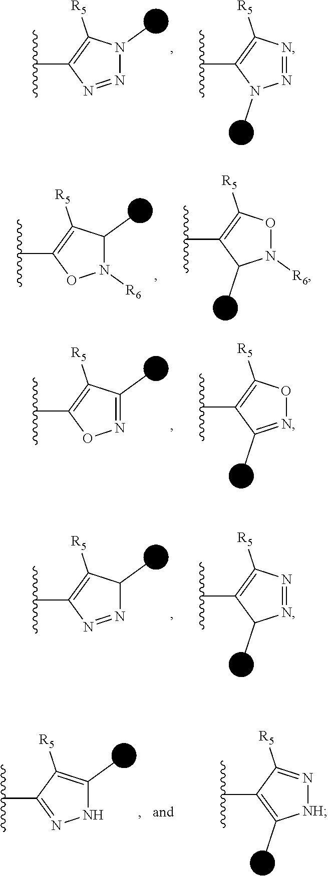

wherein R₅ is as defined herein, preferably R₅ is H; R₆ is as defined herein; and ● is as defined herein. More preferably, Z is

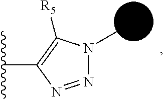

wherein Q is

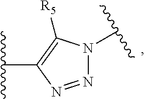

R₅ is as defined herein, preferably R₅ is H; and ● is as defined herein. Preferably, Z is

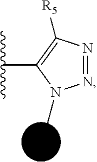

wherein Q is

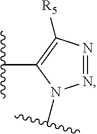

R₅ is as defined herein, preferably R₅ is H; and ● is as defined herein. Z may be

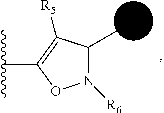

wherein Q is

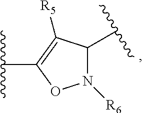

R₅ is as defined herein, preferably R₅ is H; R₆ is as defined herein; and ● is as defined herein. Z may be

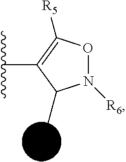

wherein Q is

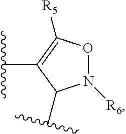

R₅ is as defined herein, preferably R₅ is H; R₆ is as defined herein; and ● is as defined herein. Z may be

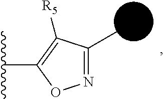

wherein Q is

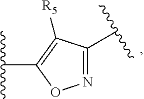

R₅ is as defined herein, preferably R₅ is H; and ● is as defined herein. Z may be

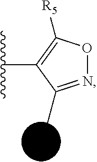

wherein Q is

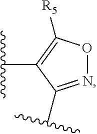

R₅ is as defined herein, preferably R₅ is H; and ● is as defined herein. Z may be

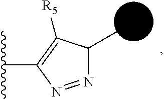

wherein Q is

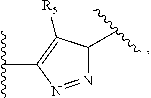

R₅ is as defined herein, preferably R₅ is H; and ● is as defined herein. Z may be

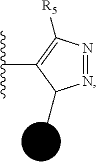

wherein Q is

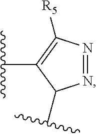

R₅ is as defined herein, preferably R₅ is H; and ● is as defined herein. Z may be

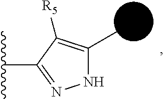

wherein Q is

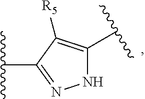

R₅ is as defined herein, preferably R₅ is H; and ● is as defined herein. Z may be

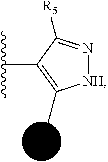

wherein Q is

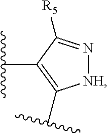

R₅ is as defined herein, preferably R₅ is H; and ● is as defined herein. Optionally, in any one of these embodiments Y is O. Optionally, in any one of these embodiments, a linker may be arranged between ● and Q.

Preferably, in any one of the methods of preparing a compound of formula (III), R₁ is

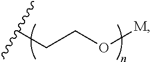

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; and Z may be selected from the group consisting of

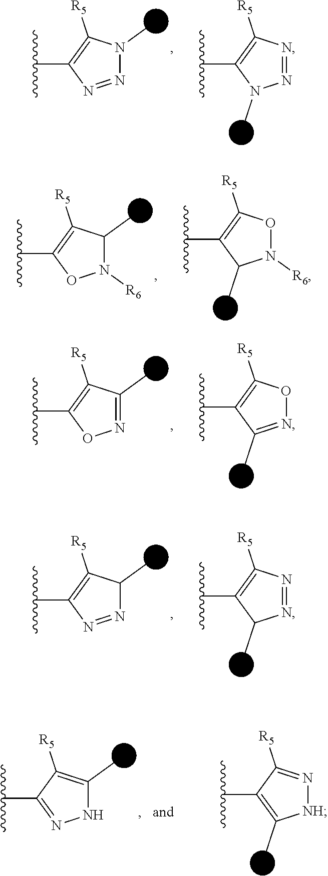

wherein R₅ is as defined herein, preferably R₅ is H; R₆ is as defined herein; and ● is as defined herein. More Preferably, Z is

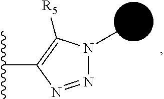

wherein Q is

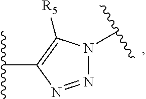

R₅ is as defined herein, preferably R₅ is H; and is as defined herein. Preferably, Z is

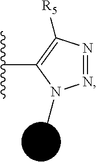

wherein Q is

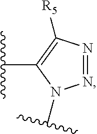

R₅ is as defined herein, preferably R₅ is H; and ● is as defined herein. Z may be

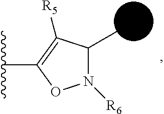

wherein Q is

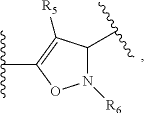

R₅ is as defined herein, preferably R₅ is H; R₆ is as defined herein; and ● is as defined herein. Z may be

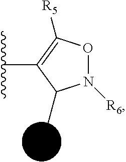

wherein Q is

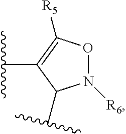

R₅ is as defined herein, preferably R₅ is H; R₆ is as defined herein; and ● is as defined herein. Z may be

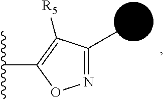

wherein Q is

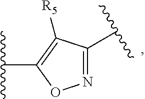

R₅ is as defined herein, preferably R₅ is H; and ● is as defined herein. Z may be

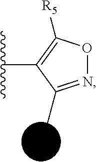

wherein Q is

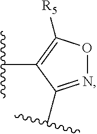

R₅ is as defined herein, preferably R₅ is H; and ● is as defined herein. Z may be

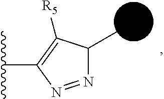

wherein Q is

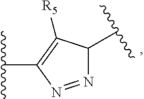

R₅ is as defined herein, preferably R₅ is H; and ● is as defined herein. Z may be

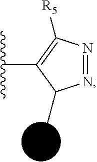

wherein Q is

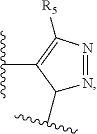

R₅ is as defined herein, preferably R₅ is H; and ● is as defined herein. Z may be

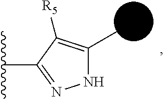

wherein Q is

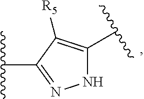

R₅ is as defined herein, preferably R₅ is H; and ● is as defined herein. Z may be

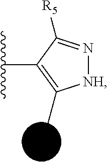

wherein Q is

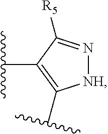

R₅ is as defined herein, preferably R₅ is H; and ● is as defined herein. Optionally, in any one of these embodiments Y is O. Optionally, in any one of these embodiments, a linker may be arranged between ● and Q.

In any one of the methods of preparing a compound of formula (III), R₁ may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

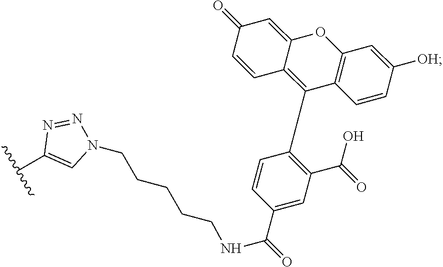 [SMILES: *c1cn(CCCCCNC(=O)c2ccc(-c3c4ccc(=O)cc-4oc4cc(O)ccc34)c(C(=O)O)c2)nn1]

or still more preferably R₁ is

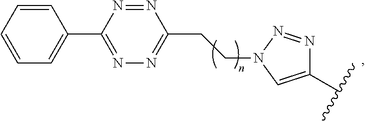

wherein n is 1, 2, 3, 4, 5, 6, 7, 8 or 9, preferably 1, 2 or 3, more preferably 1; or preferably R₁ is

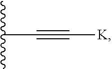

wherein K is as defined herein, preferably K is H; and Z is

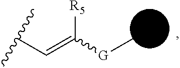

wherein Q is

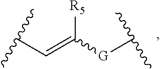

R₅ is as defined herein, preferably R₅ is H; G is S, O or NR₁₀, wherein R₁₀ is as defined herein, preferably R₁₀ is H; and ● is as defined herein. Preferably, Z is

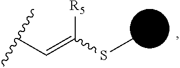

wherein Q is

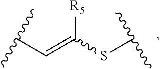

R₅ is as defined herein, preferably R₅ is H, and ● is as defined herein. R₁ may be optionally substituted aryl. Preferably, R₁ is optionally substituted phenyl. More preferably, R₁ is unsubstituted phenyl. R₁ may be optionally substituted heteroaryl. Preferably, R₁ is optionally substituted triazolyl. More preferably, R₁ is triazolyl substituted with optionally substituted C₁-C₈-alkyl. R₁ may be a fluorophore. More preferably, R₁ is triazolyl substituted with a fluorophore. Still more preferably, R₁ is

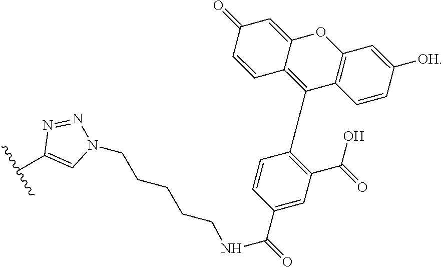 [SMILES: *c1cn(CCCCCNC(=O)c2ccc(-c3c4ccc(=O)cc-4oc4cc(O)ccc34)c(C(=O)O)c2)nn1]

Still more preferably, R₁ is

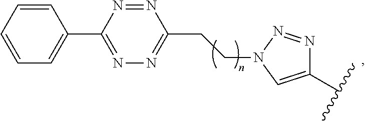

wherein n is 1, 2, 3, 4, 5, 6, 7, 8 or 9, preferably 1, 2 or 3, more preferably 1. Preferably R₁ is

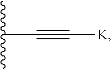

wherein K is H or C₁-C₈-alkyl, preferably H or C₁-C₆-alkyl, more preferably H or C₁-C₄-alkyl, still more preferably H or C₁-C₂-alkyl; even more preferably K is H. Preferably, in any one of these embodiments Y is a bond. Optionally, in any one of these embodiments, a linker may be arranged between ● and Q.

In any one of the methods of preparing a compound of formula (III), ● may represent an amino acid, a peptide, a protein, an antibody, a nucleotide, an oligonucleotide, a saccharide, a polysaccharide, a detectable label, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments, ● represents a protein tag. Preferably, ● represents a fluorophore such as CY₅, fluorescein or EDANS. Preferably, ● represents biotin. Preferably, ● represents a linker. Preferably, ● represents a drug. Preferably, ● represents a linker-drug conjugate. Preferably, ● represents a linker-fluorophore conjugate. In some embodiments ● represents a polymer. In some embodiments, ● represents a small molecule. In some embodiments, ● represents an optionally substituted C₁-C₈-alkyl, preferably an optionally substituted C₁-C₄-alkyl, more preferably an optionally substituted C₁-C₂-alkyl. In some embodiments, ● represents an optionally substituted phenyl. Preferably, ● represents an optionally substituted aromatic 5- or 6-membered heterocyclic system. Optionally, in any one of these embodiments a linker may be arranged between ● and Q.

In any one of the methods of preparing a compound of formula (III), ● may represent an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments ● represents a polymer. In some embodiments, ● represents an optionally substituted C₁-C₈-alkyl, preferably an optionally substituted C₁-C₄-alkyl, more preferably an optionally substituted C₁-C₂alkyl. In some embodiments, ● represents an optionally substituted phenyl. Preferably, ● represents an optionally substituted aromatic 5- or 6-membered heterocyclic system. Optionally, in any one of these embodiments a linker may be arranged between ● and Q.

Preferably, in any one of the methods of preparing a compound of formula (III), ● represents an amino acid, a peptide, a protein, an antibody, a nucleotide, or an oligonucleotide; wherein optionally a linker is arranged between ● and Q. More preferably, ● represents a peptide, a protein, an antibody, or an oligonucleotide; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. Optionally, in any one of these embodiments a linker may be arranged between ● and Q.

Preferably, in any one of the processes of preparing a compound of formula (III), ● represents a drug, a protein tag, or a fluorophore such as CY₅, fluorescein or EDANS, biotin, a protein, a peptide, an antibody or an oligonucleotide; wherein optionally a linker is arranged between ● and Q. Preferably, ● represents a drug. Preferably, ● represents a protein tag. Preferably, ● represents a linker-drug conjugate. Preferably, S represents a fluorophore such as CY₅, fluorescein or EDANS. Preferably, ● represents biotin. Preferably, ● represents a protein. Preferably, ● represents a peptide. Preferably, ● represents an antibody. Preferably, ● represents an oligonucleotide. Optionally, in any one of these embodiments a linker may be arranged between ● and Q.

Preferably, in any one of the methods of preparing a compound of formula (III), ● represents a linker, a fluorophore, or a linker-fluorophore conjugate. Preferably, ● represents a linker. Preferably, ● represents a fluorophore. Preferably, ● represents a linker-fluorophore conjugate.

Preferably, in any one of the methods of preparing a compound of formula (III), ● represents a small molecule, a fluorophore, a peptide, a protein, or an antibody; wherein optionally a linker is arranged between ● and Q. Preferably, ● represents a small molecule. Preferably, ● represents a fluorophore. Preferably, ● represents a peptide. Preferably, ● represents a protein. Preferably, ● represents an antibody. Optionally, in any one of these embodiments a linker may be arranged between ● and Q.

Preferably, in any one of the methods of preparing a compound of formula (III), ● represents a detectable label. Optionally, in this embodiment, a linker may be arranged between ● and Q.

Preferably, in any one of the methods of preparing a compound of formula (III), ● represents a linker, a drug, or a linker-drug conjugate. Preferably, ● represents a linker. Preferably, ● represents a drug. Preferably, ● represents a linker-drug conjugate.

When ● represents a linker or a linker-drug conjugate, the linker may be any chemical moiety which is capable to link a drug moiety to Q, wherein Q is as defined herein. The linker may be any linker known to a person skilled in the art. As used herein, the term “linker-drug conjugate” refers to a molecule or chemical group comprising or consisting of a linker, as defined herein, and a drug moiety. In general, in a linker drug conjugate the linker is covalently linked to the drug. As an illustrative example, the linker used in the invention may comprise a self-cleaving peptide, which may be cleaved by an enzyme, e.g. cathepsin B. In particular, the enzyme may cleave the linker to release the drug. As illustrative example, the enzyme, such as cathepsin B, may cleave the linker after uptake into a cell, such that the drug is released at the target location. In particular, the linker comprising a self-cleaving peptide used in the invention may comprise a valine-citrulline-p-aminobenzyloxycarbonyl (VC-PAB) moiety, a valine-alanine-p-aminobenzyloxycarbonyl (VA-PAB) moiety, a lysine-phenylalanine-p-aminobenzyloxycarbonyl (KF-PAB) moiety, or a valine-lysine-p-aminobenzyloxycarbonyl (VK-PAB) moiety. Linkers with a self-cleaving peptide are, for example, disclosed in U.S. patent application publication US 2006/0074008, G. M. Dubowchik et al., Bioconjugate Chem. 2002, 13, 855-869, or S. O. Doronina et al., Nature Biotechnology, vol. 21, 778-784 (2003), the whole disclosure of these documents is incorporated herein by reference.

When ● represents a linker-drug conjugate, the linker-drug conjugate may have the following general formula LD:

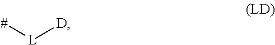

wherein:
L is a linker;
D is a drug; and
#indicates the position of Q, wherein Q is as defined herein.

Preferably, when ● represents a linker, or a linker-drug conjugate, the linker L is L1:

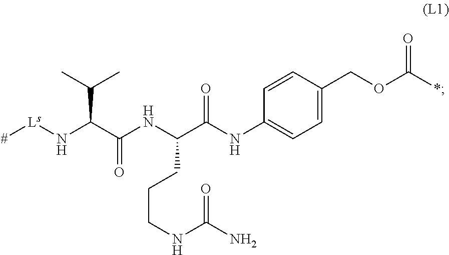

wherein:
L^{s} is an optional spacer;
#indicates the position of Q, wherein Q is as defined herein; and
* indicates the position of the drug.
Accordingly, L1 has a valine-citrulline-p-aminobenzyloxycarbonyl (VC-PAB) moiety.

The linker L may be 12:

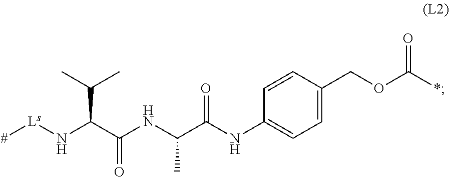

wherein:
L^{s} is an optional spacer;
#indicates the position of Q, wherein Q is as defined herein; and
* indicates the position of the drug.
Accordingly, L2 has a valine-alanine-p-aminobenzyloxycarbonyl (VA-PAB) moiety.

The linker L may be L3:

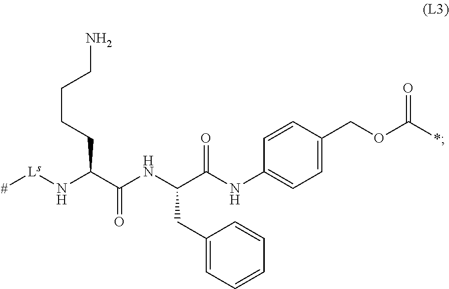

wherein:
L^{s} is an optional spacer;
#indicates the position of Q, wherein Q is as defined herein; and
* indicates the position of the drug.
Accordingly, L3 has a lysine-phenylalanine-p-aminobenzyloxycarbonyl (KF-PAB) moiety.

The linker L may be L4:

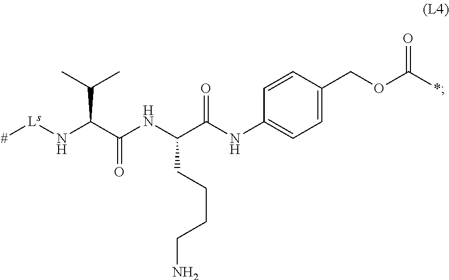

wherein:
L^{s} is an optional spacer;
#indicates the position of Q, wherein Q is as defined herein; and
* indicates the position of the drug.
Accordingly, L4 has a valine-lysine-p-aminobenzyloxycarbonyl (VK-PAB) moiety.

As indicated herein, L^{s} is an optional spacer. Accordingly, L^{s} may be present or absent. Preferably, L^{s} is present. As used herein, the term “spacer” may refer to any chemical moiety that is capable to covalentiy link the amino acid (valine or lysine in the formulae (L1) to (L4)) to Q. For this purpose, the spacer L^{s} may have a functional group, which is capable to form a bond to an amino group of an amino acid. Such functional group may be, for example, a carbonyl group, which can be depicted as, for example,

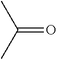

or —C(O)—. Virtually any spacer moiety (spacer) can be used. The spacer may, for example, be a straight or branched hydrocarbon-based moiety. The spacer can also comprise cyclic moieties. If the spacer is a hydrocarbon-based moiety, the main chain of the spacer may comprise only carbon atoms but can also contain heteroatoms such as oxygen (O), nitrogen (N) or sulfur (S) atoms. The spacer may for example include a C₁-C₂₀ carbon atom chain or a polyether-based chain such as a polyethylene glycol-based chain with —(O—CH₂—CH₂)— repeating units. In typical embodiments of hydrocarbon-based spacers, the spacer may comprise between 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18, and 19 main chain atoms. In some embodiments, L^{s} is selected from the group consisting of #—(C₁-C₁₀)alkylene-C(O)—**, #—(C₃-C₈)carbocyclo-C(O)—**, #-arylene-C(O)—**, #—(C₁-C₁₀)alkylene-arylene-C(O)—**, #-arylene-(C₁-C₁₀)alkylene-C(O)—**, #—(C₁-C₁₀)alkylene-(C₃-C₈)carbocyclo-C(O)—**, #—(C₃-C₈)carbocyclo-(C₁-C₁₀)alkylene-C(O)**, #—(C₃-C₈)heterocyclo-C(O)—**, #—(C₁-C₁₀)alkylene-(C₃-C₈)heterocyclo-C(O)—**, #—(C₃-C₈)heterocyclo-(C₁-C₁₀)alkylene-C(O)—**, #—(CH₂CH₂CH₂)ᵣ—C(O)—**, #—(CH₂CH₂CH₂)ᵣ(CH₂)₅—C(O)—*, #—(CH₂CH₂NH)ᵣ—C(O)—**, #—(CH₂CH₂NH)ᵣ—(CH₂)₅—C(O)—**, #—(CH₂CH₂O)ᵣ—C(O)—**, and #—(CH₂CH₂O)ᵣ—(CH₂)ₛ—C(O)—**, wherein r, in each instance, is an integer ranging from 1 to 20, preferably 1 to 10, more preferably 2 to 8, still more preferably 3 to 6, even more preferably r is 4; and s, in each instance, is an integer ranging from 1 to 10, preferably 1 to 6, more preferably 1 to 4, still more preferably 1 to 3, even more preferably s is 2; #indicates the position of Q, and ** indicates the position of the amino acid (valine or lysine in the formulae (L1) to (L4)).

Preferably, when ● represents a linker, or a linker-drug conjugate, the linker L is L1*:

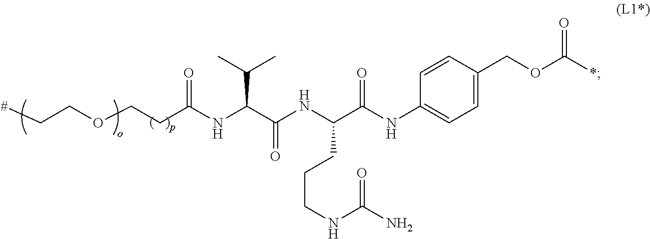

wherein:
o is an integer ranging from 1 to 20, preferably 1 to 10, more preferably 2 to 8, still more preferably 3 to 6, even more preferably 0 is 4;
p is an integer ranging from 0 to 9, preferably 0 to 5, more preferably 0 to 3, still more preferably 0 to 2, even more preferably p is 1;
#indicates the position of Q, wherein Q is as defined herein; and
* indicates the position of the drug.

The linker L may be L2*:

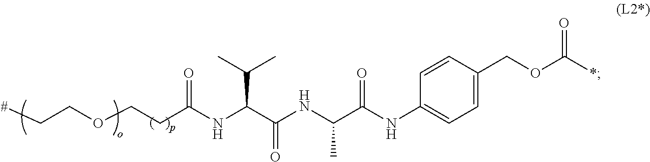

wherein:
o is an integer ranging from 1 to 20, preferably 1 to 10, more preferably 2 to 8, still more preferably 3 to 6, even more preferably o is 4;
p is an integer ranging from 0 to 9, preferably 0 to 5, more preferably 0 to 3, still more preferably 0 to 2, even more preferably p is 1;
#indicates the position of Q, wherein Q is as defined herein; and
* indicates the position of the drug.

The linker L may be L3*:

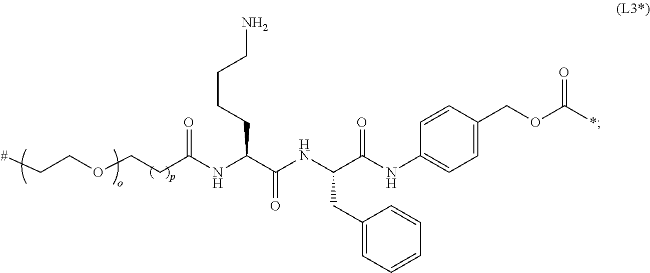

wherein:
is an integer ranging from 1 to 20, preferably 1 to 10, more preferably 2 to 8, still more preferably 3 to 6, even more preferably o is 4;
p is an integer ranging from 0 to 9, preferably 0 to 5, more preferably 0 to 3, still more preferably 0 to 2, even more preferably p is 1;
#indicates the position of Q, wherein Q is as defined herein; and
* indicates the position of the drug.

The linker L may be L4*:

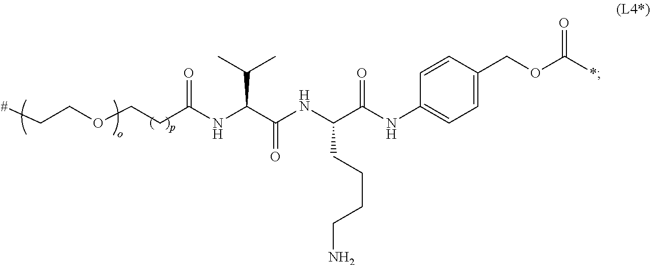

wherein:
o is an integer ranging from 1 to 20, preferably 1 to 10, more preferably 2 to 8, still more preferably 3 to 6, even more preferably o is 4;
p is an integer ranging from 0 to 9, preferably 0 to 5, more preferably 0 to 3, still more preferably 0 to 2, even more preferably p is 1;
#indicates the position of Q, wherein Q is as defined herein; and
* indicates the position of the drug.

In some embodiments, when ● represents a linker-drug conjugate, the linker-drug conjugate LD may be LD1:

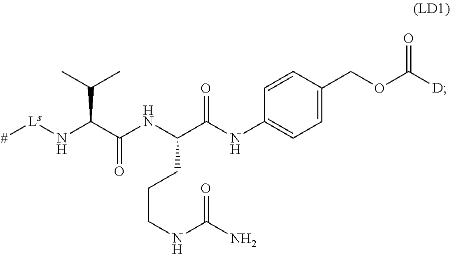

wherein:
L^{s} is an optional spacer;
#indicates the position of Q, wherein Q is as defined herein; and
D is a drug.

The linker-drug conjugate LD may be LD2:

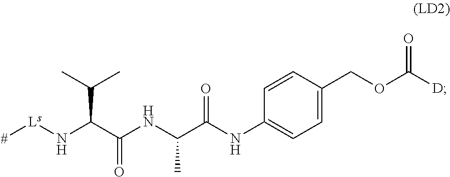

wherein:
L^{s} is an optional spacer;
#indicates the position of Q, wherein Q is as defined herein; and
D is a drug.

The linker-drug conjugate LD may be LD3:

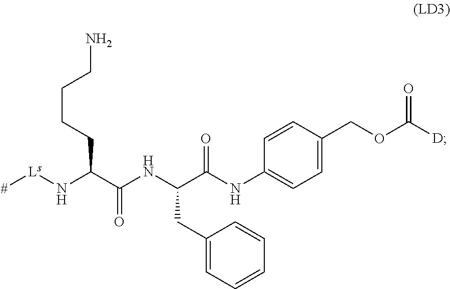

wherein:
L^{s} is an optional spacer;
#indicates the position of Q, wherein Q is as defined herein; and
D is a drug.

The linker-drug conjugate LD may be LD4:

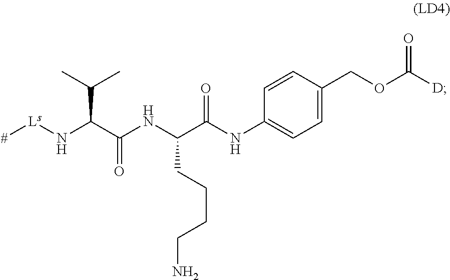

wherein:
L^{s} is an optional spacer;
#indicates the position of Q, wherein Q is as defined herein; and
D is a drug.

Preferably, when ● represents a linker, or a linker-drug conjugate, the linker-drug conjugate LD is LD1*:

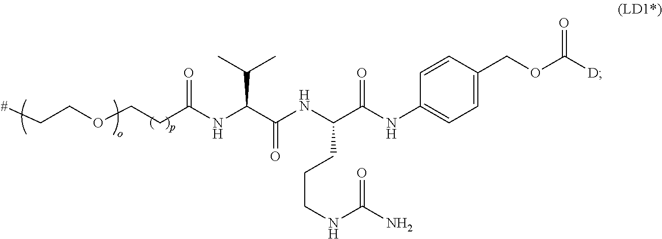

wherein:
o is an integer ranging from 1 to 20, preferably 1 to 10, more preferably 2 to 8, still more preferably 3 to 6, even more preferably o is 4;
p is an integer ranging from 0 to 9, preferably 0 to 5, more preferably 0 to 3, still more preferably 0 to 2, even more preferably p is 1;
#indicates the position of Q, wherein Q is as defined herein; and
D is a drug.

The linker-drug conjugate LD may be LD2*:

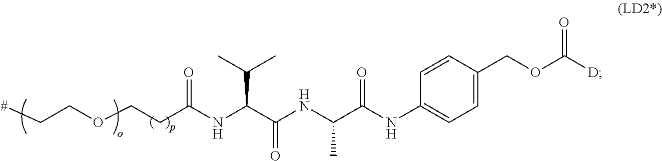

wherein:
o is an integer ranging from 1 to 20, preferably 1 to 10, more preferably 2 to 8, still more preferably 3 to 6, even more preferably o is 4;
p is an integer ranging from 0 to 9, preferably 0 to 5, more preferably 0 to 3, still more preferably 0 to 2, even more preferably p is 1;
#indicates the position of Q, wherein Q is as defined herein; and
D is a drug.

The linker-drug conjugate LD may be LD3*:

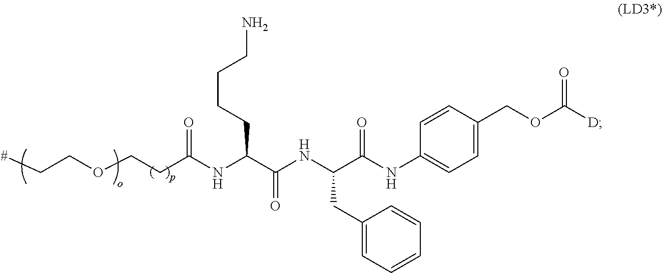

wherein:
o is an integer ranging from 1 to 20, preferably 1 to 10, more preferably 2 to 8, still more preferably 3 to 6, even more preferably o is 4;
p is an integer ranging from 0 to 9, preferably 0 to 5, more preferably 0 to 3, still more preferably 0 to 2, even more preferably p is 1;
#indicates the position of Q, wherein Q is as defined herein; and
D is a drug.

The linker-drug conjugate LD may be LD4*:

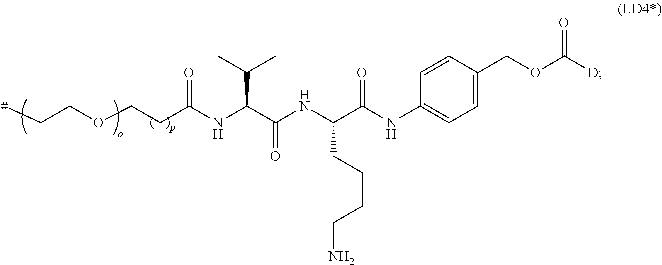

wherein:
o is an integer ranging from 1 to 20, preferably 1 to 10, more preferably 2 to 8, still more preferably 3 to 6, even more preferably o is 4;
p is an integer ranging from 0 to 9, preferably 0 to 5, more preferably 0 to 3, still more preferably 0 to 2, even more preferably p is 1;
#indicates the position of Q, wherein Q is as defined herein; and
D is a drug.

The term “drug” (which may be also denoted as “drug D”, or “drug moiety”, or “drug moiety D”) used in the present invention may be any drug known to a person skilled in the art. As an illustrative example, the drug may be a cytostatic or a cytotoxic drug. As an illustrative example, a drug used in the invention may be an auristatin, preferably monomethyl auristatin E (MMAE) or monomethyl auristatin F (MMAF).

Accordingly, in some embodiments, the drug D is monomethyl auristatin F (MMAF). MMAF is represented by the following structural formula:

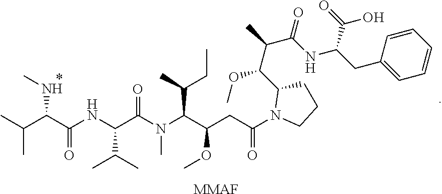

Monomethyl auristatin F (MMAF) may be bound to the linker via the nitrogen atom marked with an asterisk (*).

In some embodiments the drug D is monomethyl auristatin E (also known as MMAE). MMAE is represented by the following structural formula:

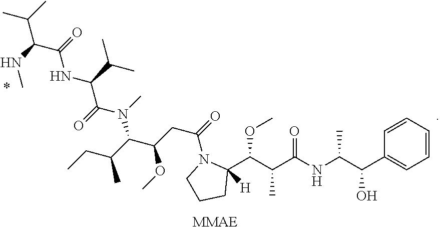

Monomethyl auristatin E (MMAE) may be bound to the linker via the nitrogen atom marked with an asterisk (*).

When ● represents a linker, a drug or a linker-drug conjugate, Z may be any Z as defined herein, wherein ● represents a linker, a drug or a linker-drug conjugate. Accordingly, Z may be any

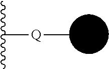

as defined herein, wherein ● represents a linker, a drug or a linker-drug conjugate; and Q may be any Q as defined herein. In some embodiments, when ● represents a linker, a drug or a linker-drug conjugate, Q may be a five- or six-membered heterocyclic moiety comprising 1, 2 or 3 heteroatoms independently selected from the group consisting of N, O or S. In some embodiments, Z may be

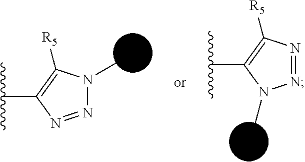

wherein R₅ is as defined herein, preferably R₅ is H. Accordingly, Z may, be

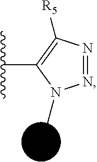

wherein Q is

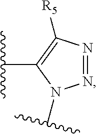

and R₅ is as defined herein, preferably R₅ is H. In preferred embodiments, when ● represents a linker, a drug or a linker-drug conjugate, Z is

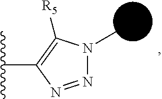

wherein Q is

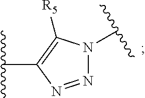

and R₅ is as defined herein, preferably R₅ is H.

In some embodiments, when ● represents a linker, a drug or a linker-drug conjugate, R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with hydroxy-(C₁—C-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. In particular, when ● represents a linker, a drug or a linker-drug conjugate, R₁ may be

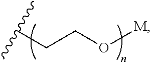

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; wherein the wavy line indicates the attachment point to the Y. In these embodiments, the Y may be any Y as defined herein. Preferably, in any one of these embodiments, Y is O (oxygen).

In other embodiments, when ● represents a linker, a drug or a linker-drug conjugate, R₁ may be optionally substituted C₁-C₈-alkyl, preferably methyl, ethyl, propyl or butyl, more preferably methyl or ethyl, still more preferably ethyl. In these embodiments, the Y may be any Y as defined herein. Preferably, in any one of these embodiments, Y is O (oxygen).

In some embodiments, when ● represents a linker, a drug or a linker-drug conjugate [symbol] may represent an antibody.

In any one of the methods of preparing a compound of formula (III), [symbol] may represent an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system. Preferably, [symbol] represents an amino acid, a peptide, a protein, an antibody, a nucleotide, an oligonucleotide, or a small molecule. More preferably, [symbol] represents a peptide, a protein, an antibody, an oligonucleotide, or a small molecule. Preferably, [symbol] represents an amino acid. Preferably, [symbol] represents a peptide. Preferably, [symbol] represents a protein. Preferably, [symbol] represents an antibody. Preferably, [symbol] represents a nucleotide. Preferably, [symbol] represents an oligonucleotide. In some embodiments [symbol] represents a saccharide. In some embodiments [symbol] represents a polysaccharide. In some embodiments [symbol] represents a polymer. Preferably, [symbol] represents a small molecule. In some embodiments [symbol] represents an optionally substituted C₁-C₈-alkyl, preferably an optionally substituted C₁-C₆-alkyl, more preferably an optionally substituted C₁-C₄-alkyl, still more preferably an optionally substituted C₁-C₂-alkyl. In some embodiments [symbol] represents an optionally substituted C₃-C₈-alkyl, preferably an optionally substituted C₃-C₆-alkyl, more preferably an optionally substituted C₃-C₄-alkyl. In some embodiments [symbol] represents an optionally substituted C₅-C₈-alkyl, preferably an optionally substituted C₆-C₇-alkyl. In some embodiments represents an optionally substituted phenyl. In some embodiments represents an optionally substituted aromatic 5- or 6-membered heterocyclic system.

Preferably, in any one of the methods of preparing a compound of formula (III), [symbol] represents an antibody, preferably an IgG antibody, such as e.g. a Cetuximab or a Trastuzumab or a Brentuximab; a protein, preferably a GFP protein or eGFP-protein, an mCherry protein or an albumin; a small molecule; a peptide, preferably a peptide of formula (VIII)

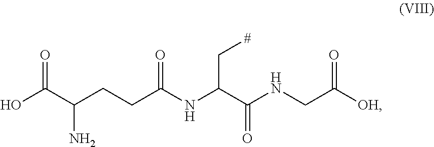

or of formula (IX).

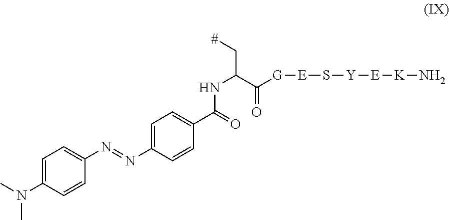

wherein #represents the position of S. Preferably, [symbol] represents an antibody, such as e.g. a Cetuximab or a Trastuzumab or a Brentuximab. Preferably, [symbol] represents a protein, such as e.g. a GFP protein or eGFP-protein. In some embodiments [symbol] represents an mCherry protein. In some embodiments [symbol] represents albumin. Preferably, [symbol] represents a small molecule. Preferably, [symbol] represents a peptide. More preferably, [symbol] represents a peptide of formula (VIII)

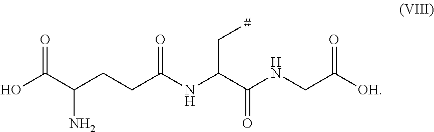

More preferably, [symbol] represents a peptide of formula (IX)

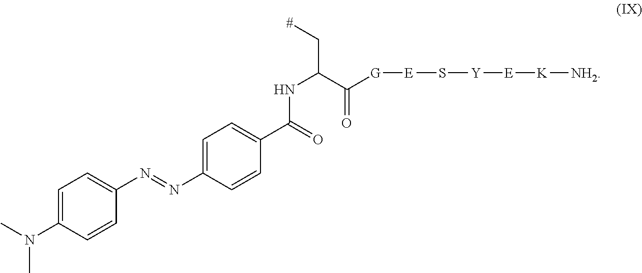

In preferred embodiments of any one of the processes of the invention, [symbol] represents an antibody (e.g. a Cetuximab, Trastuzumab, or Brentuximab) and ● represents a protein tag, or a fluorophore such as CY₅, fluorescein or EDANS, biotin, a peptide, a protein, an oligonucleotide, or a small molecule; wherein optionally a linker is arranged between ● and Q. Preferably, [symbol] represents an antibody and ● represents a protein tag. Preferably, [symbol] represents an antibody and ● represents a fluorophore such as CY₅, fluorescein or EDANS. Preferably, [symbol] represents an antibody and ● represents biotin. Preferably, [symbol] represents an antibody and ● represents a peptide. Preferably, [symbol] represents an antibody and ● represents a protein. Preferably, [symbol] represents an antibody and ● represents an oligonucleotide. Preferably, [symbol] represents an antibody and ● represents a small molecule. Optionally, in any one of these embodiments a linker may be arranged between ● and Q.

Preferably, in any one of the methods of preparing a compound of formula (III), [symbol] represents a protein (e.g. a GFP protein or eGFP protein or mCherry proterin) and ● represents a protein tag, or a fluorophore such as CY₅, fluorescein or EDANS, biotin, a peptide, an antibody, a protein, an oligonucleotide, or a small molecule; wherein optionally a linker is arranged between ● and Q. Preferably, [symbol] represents a protein and ● represents a protein tag. Preferably, [symbol] represents a protein and ● represents a fluorophore such as CY₅, fluorescein or EDANS. Preferably, [symbol] represents a protein and ● represents biotin. Preferably, [symbol] represents a protein and ● represents a peptide. Preferably, [symbol] represents a protein and ● represents an antibody. Preferably, [symbol] represents a protein and ● represents a protein. Preferably, [symbol] represents a protein and ● represents an oligonucleotide. Preferably, [symbol] represents a protein and ● represents a small molecule Optionally, in any one of these embodiments a linker may be arranged between ● and Q.

Preferably, in any one of the methods of preparing a compound of formula (III), [symbol] represents a peptide and ● represents a protein tag, or a fluorophore such as CY₅, fluorescein or EDANS, biotin, a peptide, a protein, an oligonucleotide, or a small molecule; wherein optionally a linker is arranged between ● and Q. Preferably, [symbol] represents a peptide and ● represents a protein tag. Preferably, [symbol] represents a peptide and ● represents a fluorophore such as CY₅ or EDANS. Preferably, [symbol] represents a peptide and ● represents biotin. Preferably, [symbol] represents a peptide and ● represents a peptide. Preferably, [symbol] represents a peptide and ● represents a protein. Preferably, [symbol] represents a peptide and ● represents an oligonucleotide. Preferably, [symbol] represents a peptide and ● represents a small molecule. Optionally, in any one of these embodiments a linker may be arranged between ● and Q.

Preferably, in any one of the methods of preparing a compound of formula (III), [symbol] represents an amino acid and ● represents a protein tag, or a fluorophore such as CY₅, fluorescein or EDANS, biotin, a peptide, a protein, an oligonucleotide, or a small molecule; wherein optionally a linker is arranged between ● and Q. Preferably, [symbol] represents an amino acid and ● represents a protein tag. Preferably, [symbol] represents an amino acid and ● represents a fluorophore such as CY₅, fluorescein or EDANS. Preferably, [symbol] represents an amino acid and ● represents biotin. Preferably, [symbol] represents an amino acid and ● represents a peptide. Preferably, [symbol] represents an amino acid and ● represents a protein. Preferably, [symbol] represents an amino acid and ● represents an oligonucleotide. Preferably, [symbol] represents an amino acid and ● represents a small molecule. Optionally, in any one of these embodiments a linker may be arranged between ● and Q.

Preferably, in any one of the methods of preparing a compound of formula (III), [symbol] represents an antibody (e.g. a Cetuximab, a Trastuzumab, or a Brentuximab) and ● represents a linker, a drug, or a linker-drug conjugate. Preferably, [symbol] represents an antibody and ● represents a linker. Preferably, [symbol] represents an antibody and ● represents a drug. Preferably, [symbol] represents an antibody and ● represents a linker-drug conjugate. The linker, drug or linker-drug conjugate may be any linker, drug or linker-drug conjugate as described herein. In particular, the linker, drug, or linker-drug conjugate may be any linker, drug or linker drug conjugate as described herein with regard to embodiments where ● represents a linker, a drug or a linker-drug conjugate.

Preferably, in any one of the methods of preparing a compound of formula (III), [symbol] represents an antibody (e.g. a Cetuximab, a Trastuzumab, or a Brentuximab) and ● represents a linker, a fluorophore, or a linker-fluorophore conjugate. Preferably, [symbol] represents an antibody and ● represents a linker. Preferably, [symbol] represents an antibody and ● represents a fluorophore. Preferably, [symbol] represents an antibody and ● represents a linker-fluorophore conjugate.

Preferably, in any one of the method of preparing a compound of formula (III), [symbol] represents a nucleotide and ● represents a peptide, a protein, a protein tag, an antibody, an oligonucleotide, a fluorophore such as CY₅, fluorescein, or EDANS, biotin, or a small molecule; wherein optionally a linker is arranged between ● and Q. Preferably, [symbol] represents a nucleotide and ● represents a peptide. Preferably [symbol] represents a nucleotide and ● represents a protein. Preferably, [symbol] represents a nucleotide and ● represents a protein tag. Preferably, [symbol] represents a nucleotide and ● represents an antibody. Preferably, [symbol] represents a nucleotide and ● represents an oligonucleotide. Preferably, [symbol] represents a nucleotide and ● represents a fluorophore such as CY₅, fluorescein or EDANS. Preferably, [symbol] represents a nucleotide and ● represents biotin. Preferably, [symbol] represents a nucleotide and ● represents a small molecule. Optionally, in any one of these embodiments a linker may be arranged between ● and Q.

Preferably, in any one of the methods of preparing a compound of formula (III), [symbol] represents a nucleotide and ● represents a linker.

Preferably, in any one of the methods of preparing a compound of formula (III), [symbol] represents an oligonucleotide and ● represents a peptide, a protein, a protein tag, an antibody, an oligonucleotide, a fluorophore such as CY₅, fluorescein or EDANS, biotin, or a small molecule; wherein optionally a linker is arranged between ● and Q. Preferably, [symbol] represents an oligonucleotide and ● represents a peptide. Preferably, [symbol] represents an oligonucleotide and ● represents a protein. Preferably, [symbol] represents an oligonucleotide and ● represents a protein tag. Preferably, [symbol] represents an oligonucleotide and ● represents an antibody. Preferably, [symbol] represents an oligonucleotide and ● represents an oligonucleotide. Preferably, [symbol] represents an oligonucleotide and ● represents a fluorophore such as CY₅, fluorescein, or EDANS. Preferably, [symbol] represents an oligonucleotide and ● represents biotin. Preferably, [symbol] represents an oligonucleotide and ● represents a small molecule. Optionally, in any one of these embodiments a linker may be arranged between ● and Q.

Preferably, in any one of the processes of preparing a compound of formula (III), [symbol] represents an oligonucleotide and ● represents a linker.

In some embodiments of the method of preparing a compound of formula (III), ● represents an amino acid, a peptide, a nucleotide or an oligonucleotide, wherein the amino acid, peptide, nucleotide or oligonucleotide is bound to a solid support. In some embodiments ● represents an amino acid or a peptide bound to a solid support. In some embodiments ● represents a nucleotide or an oligonucleotide bound to a solid support. Preferably, ● represents a peptide bound to a solid support. Compounds of formula (III) of the present invention are stable under acidic conditions which are typically used for cleavage of a peptide from the solid support, e.g. 90% trifluoroacetic acid (TFA). The solid support may be any solid support known to a person skilled in the art which is suitable for solid phase peptide synthesis, or any solid support which is suitable for solid phase oligonucleotide synthesis. Such solid supports are also known as resins. Illustrative examples for a solid support suitable for solid phase peptide synthesis include organic and inorganic supports such as a Merrifield polystyrene resin (copolymer from styrene and 1-2% divinylbenzene), polyacrylamide resins, TentaGel (a graft polymer where polythyleneglycol is grafted to polystyrene), Wang resin (typically based on crosslinked polystyrene, such as in a Merrifield resin), or porous glass having defined pore size as an example for an inorganic solid support. Illustrative examples for commercially available solid supports for solid phase peptide synthesis are Rink amide resins or NovaSyn®TGR resins supplied by Merck Millipore. Illustrative examples for a solid support suitable for solid phase oligonucleotide synthesis include glass having defined pore size (controlled pore glass, CPG) and polystyrene, such as macroporous polystyrene (MPPS). Optionally, in the foregoing embodiments where the amino acid, peptide, nucleotide or oligonucleotide is bound to a solid support, a linker may be arranged between ● and Q. Accordingly, the ●, Q, linker and solid support may be arranged as follows: Q-Linker-Amino Acid-Solid Support, Q-Linker-Peptide-Solid Support, Q-Linker-Nucleotide-Solid Support, or Q-Linker-Oligonucleotide-Solid Support. The “Linker” can be virtually any linker, and the linker is arranged between ● and Q. The Linker may be any linker known to a person skilled in the art, for example, a peptidic linker or a straight or branched hydrocarbon-based moiety. The linker can also comprise cyclic moieties. A peptidic linker may comprise, for example, 1 to 50, 1 to 40, 1 to 30, 1 to 20, 1 to 10, 1 to 5, 1 to 3, or 2, or 1 amino acid(s). If the linker is a hydrocarbon-based moiety, the main chain of the linker may comprise only carbon atoms but can also contain heteroatoms such as oxygen (O), nitrogen (N) or sulfur (S) atoms, and/or contain carbonyl groups (C═O). The linker may be, for example, a C₁-C₂₀ carbon atom chain or a polyether based chain such as a polyethylene glycol-based chain with —(O—CH₂—CH₂)— repeating units. In typical embodiments of hydrocarbon-based linkers, the linking moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. A person skilled in the art knows to select suitable linkers.

In some embodiments of the method of preparing a compound of formula (III), [symbol] represents an amino acid, a peptide, a nucleotide or an oligonucleotide, wherein the amino acid, peptide, nucleotide or oligonucleotide is bound to a solid support. In some embodiments [symbol] represents an amino acid or a peptide bound to a solid support. In some embodiments [symbol] represents a nucleotide or an oligonucleotide bound to a solid support. Preferably, [symbol] represents a peptide bound to a solid support.

In some embodiment of the method of preparing a compound of formula (III), the

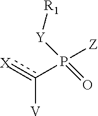

and the [symbol] -SH are in the same molecule. Accordingly, the present invention also relates to a method wherein a compound of formula (L)

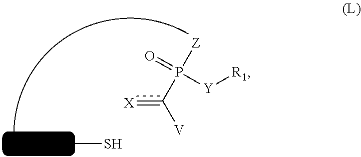

wherein the

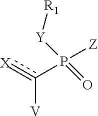

and the [symbol] -SH are in the same molecule as indicated by the arc connecting the Z and the [symbol] , is reacted to give a compound of formula (IIIa):

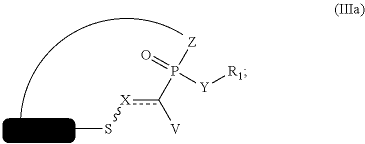

wherein [symbol] represents a bond if [symbol] in a compound of formula (L) represents a double bond, and X represents

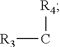

or [symbol] represents a double bond if [symbol] in a compound of formula (L) represents a triple bond, and X represents R₃—C; and [symbol] , R₁, R₃, R₄ V, Y and Z are as defined herein. In some embodiments the compound (L) having the

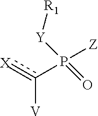

and the [symbol] -SH in the same molecule is a peptide, such as for example the BCL9 peptide. Accordingly, the compound of formula (IIIa) obtained by the process may be a cyclic peptide, such as for example a cyclic peptide derived from the BCL9 peptide. All methods described herein for compounds of formula (I), (II) and (III) can be carried out analogously for compounds of formula (L) and (IIIa).

### Method of Preparing a Conjugate of an Antibody Molecule

The present invention also relates to a method of preparing a conjugate of an antibody molecule, said method comprising:
reducing at least one disulfide bridge of an antibody molecule in the presence of a reducing agent; and
reacting said antibody molecule with a compound of formula (IV*)

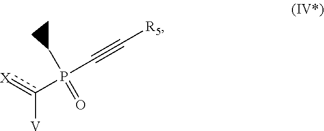

[symbol] represents a triple bond or a double bond;
V is absent when [symbol] is a triple bond; or
V represents H or C₁-C₈-alkyl when [symbol] is a double bond;
X represents R₃—C when is a triple bond; or
X represents

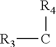

when [symbol] is a double bond;
▴ represents

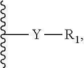

wherein [symbol] indicates the attachment point to the phosphorus; or
▴ represents Z;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
R₅ represents H or C₁-C₈-alkyl; and
Z represents a residue bound to the phosphorus via a carbon atom and comprising a group ●, wherein ● represents an optionally substituted aliphatic or optionally substituted aromatic residue
resulting in a conjugate of an antibody molecule comprising at least one moiety of formula (V)

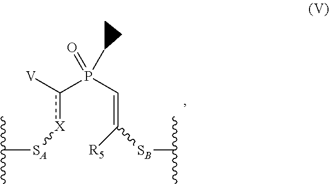

wherein S_{A} and S_{B} are each sulfur atoms of a chain of the antibody molecule;
[symbol] represents a double bond when [symbol] in a compound of formula (IV*) represents a triple bond; or
[symbol] represents a bond when [symbol] in a compound of formula (IV*) represents a double bond;
V is absent when [symbol] is a double bond; or
V represents H or C₁-C₈-alkyl when [symbol] is a bond;
X represents R₃—C when [symbol] is a double bond; or
X represents

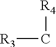

when [symbol] is a bond;
▴ represents

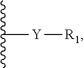

wherein [symbol] indicates the attachment point to the phosphorus; or
▴ represents Z; and
wherein R₁, R₃, R₄, R₅, Y and Z are as defined for the compound of formula (IV*).

The [symbol] , V, X, Y, R₁, R², R₃, R₄, R₅, Z, ● and [symbol] may be as defined herein for any one of the methods, compounds and/or conjugates. Any [symbol] , [symbol] , V, X, Y, R₁, R², R₃, R₄, R₅, Z, ●, and [symbol] as defined herein for any one of the methods, compounds and/or conjugates may be combined with each other.

The present invention provides a method of preparing conjugates of antibody molecules by reaction with unsaturated phosphorus(V) compounds, which includes reducing a disulfilde bridge of an antibody molecule by employing a reducing agent to obtain the two corresponding sulfhydryl groups, and then rebridging of the sulfhydryl groups by reacting with the compound of formula (IV*) to give the conjugate of an antibody molecule. The methods described herein allow to combine a huge amount of different organic residues in position ▴, and thus R₁, Z and ●, with an antibody molecule. For example, the methods according to the invention allow for conjugation of an antibody with a complex molecule, e.g. a fluorophore. The obtained conjugates are highly stable, in particular under physiologically relevant conditions, such as for example, in human serum, in the presence of small thiols, and under conditions inside of a living cell. The conjugation works under a broad variety of reaction conditions, for example, under physiologically relevant conditions, such as e.g. physiological pH. As shown in the below examples, in a conjugate of an antibody molecule as described herein, the antibody may remain specific for the target, i.e. it may retain its target selectivity (see e.g. below Example 7).

In any one of the methods of preparing a conjugate of an antibody molecule, the antibody molecule may be selected from the group consisting of an IgA, an IgD, an IgE, an IgG, an IgM, a human antibody, a humanized antibody, a chimeric antibody, a monoclonal antibody, and an isolated antibody. Accordingly, the antibody molecule may be an IgA. The antibody molecule may be an IgD. The antibody molecule may be an IgE. The antibody molecule may be an IgM. The antibody molecule may be a human antibody. The antibody molecule may be a humanized antibody. The antibody molecule may be a chimeric antibody. The antibody molecule may be a monoclonal antibody. The antibody molecule may be an isolated antibody. Preferably, the antibody molecule is an IgG, such as e.g. a Trastuzumab, a Cetuximab or a Brentuximab. The compounds of formula (IV*) offer the potential to modify all four interchain disulfides of an IgG, which allows to provide a precise antibody-to-cargo ratio of four. In particular, the method of the present invention allows at least for formation of an inter-chain cross-link between the heavy and the light chain by the moiety of formula (V). Such cross-link of a heavy chain with the light chain can be formed, e.g., between cysteins at positions C226 and C229 in human IgG. By applying the methods described herein, also a half antibody molecule can be formed which consists of one antibody heavy chain and one antibody light chain (see e.g. below Examples 7 and 11).

In any one of the methods of preparing an antibody molecule, the reducing agent may be selected from the group consisting of tris(2-carboxyethyl)phosphine (TCEP), dithiothreitol (DTT), sodium dithionite, sodium thiosulfate, and sodium sulfite. Accordingly, the reducing agent may be dithiothreitol (DTT). The reducing agent may be sodium dithionite. The reducing agent may be sodium sulfite. Preferably, the reducing agent is tris(2-carboxyethyl)phosphine (TCEP).

Preferably, in any one of the methods of preparing a conjugate of an antibody molecule, [symbol] represents a triple bond; V is absent; X represents R₃—C, R₃ represents H or C₁-C₈-alkyl; R₅ represents H or C₁-C₈alkyl; and [symbol] represents a double bond. Preferably, R₃ represents H or C₁-C₆-alkyl, more preferably H or C₁-C₄-alkyl, still more preferably H or C₁-C₂-alkyl. Even more preferably, R₃ is H. Preferably, R₅ represents H or C₁-C₆alkyl, more preferably R₅ represents H or C₁-C₄-alkyl, still more preferably R₅ represents H or C₁-C₂-alkyl. Even more preferably, R₅ is H. Preferably, in any one of the methods of preparing a conjugate of an antibody molecule, when [symbol] is a triple bond and X is R₃—C, R₃ and R₅ are the same; more preferably, when [symbol] is a triple bond and X is R₃—C, R₃ and R₅ are both H.

In some embodiments, in any one of the methods of preparing a conjugate of an antibody molecule, [symbol] may represent a double bond; V may be H or C₁-C₈-alkyl; X may represent

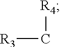

R₃ and R₄ may independently represent H or C₁-C₈-alkyl; R₅ represents H or C₁-C₈-alkyl; and [symbol] may represents a bond. Preferably, R₃ and R₄ independently represent H or C₁-C₆-alkyl, more preferably H or C₁-C₄-alkyl, still more preferably H or C₁-C₂-alkyl. Preferably, R₃ and R₄ are the same. More preferably, R₃ and R₄ are both H. Preferably, V is H or C₁-C₆-alkyl, more preferably H or C₁-C₄-alkyl, still more preferably H or C₁-C₂-alkyl. Even more preferably, V is H. In preferred embodiments, R₃, R₄ and V are the same; more preferably, R₃, R₄ and V are each H. Preferably, in any one of the methods of preparing a conjugate of an antibody molecule, when [symbol] is a double bond and X is

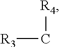

R₃, R₄ and R₅ are the same; more preferably, R₃, R₄, R₅ and V are the same. More preferably, when [symbol] is a double bond and X is

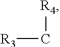

R₃, R₄ and R₅ are each H; even more preferably, R₃, R₄, R₅ and V are each H.

With regard to the representations [symbol] and [symbol] used herein, it is noted that, as commonly known to a person skilled in the art, each carbon atom is tetravalent. Accordingly, a structure

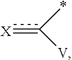

wherein X and V are as defined herein and the asterisk (*) indicates attachment to the phosphorus, includes the structures

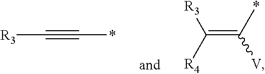

wherein R₃, R₄ and V are as defined herein. A structure

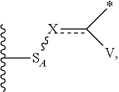

wherein S_{A}, X and V are as defined herein and the asterisk (*) indicates attachment to the phosphorus, includes the structures

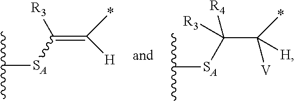

wherein R₃, R₄, S_{A} and V are as defined herein, and H is hydrogen.

In any one of the methods of preparing a conjugate of an antibody molecule, ▴ may represent

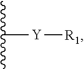

wherein [symbol] indicates the attachment point to the phosphorus; Y represents O, NR², S or a bond; R₁ represents an optionally substituted aliphatic or optionally substituted aromatic residue; and R² represents H or C₁-C₈-alkyl. Preferably, in each instance, R₁ is bound to Y via a carbon atom.

Accordingly, Y may be oxygen (O).

Y may be NR². R² is H or C₁-C₈-alkyl. Preferably, R² is C₁-C₈-alkyl. More preferably, R² is methyl, ethyl, propyl or butyl. Still more preferably, R² is methyl or ethyl.

Y may be S (sulfur).

Y may be a bond. In particular; Y may be a single bond which connects R₁ with the phosphorus.

R₁, whenever mentioned throughout the present specification, may be any aliphatic or aromatic residue, which can be optionally substituted, and which does not interfere with the method of preparing a conjugate of an antibody molecule as described herein. Accordingly, R₁ covers a broad spectrum of aliphatic or aromatic residues, such as e.g. a group adjusting the water-solubility (e.g. an ethyleneglycol oligomer), a group which can be used for further functionalization (e.g. a group comprising a carbon-carbon triple bond which can be further functionalized, e.g., a so-called “click-handle” which can be further functionalized by a 1,3-dipolar cycloaddition), or a fluorophore (see, e.g., below Examples 2 and 7). A person skilled in the art knows to select suitable residues R₁ which are compatible with the methods described herein.

In any one of the methods of preparing a conjugate of an antibody molecule, R₁ may represent a small molecule; C₁-C₈-alkyl optionally substituted with at least one of (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; preferably in in each instance Y is O. Accordingly, R₁ may be a small molecule. R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with at least one of F, Cl, Br, I, —NO₂, —N(C₁-C₈-alkyl)H, —NH₂, —N₃, —N(C₁-C₈-alkyl)₂, ═O, C₃-C₈-cycloalkyl, and/or —S—S—(C₁-C₈-alkyl). R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with C₂-C₈-alkenyl. R₁ may be C₁-C₈-alkyl optionally substituted with C₂-C₈-alkynyl. Preferably, in any one of these embodiments Y is O.

In any one of the methods of preparing a conjugate of an antibody molecule, R₁ may represent phenyl optionally independently substituted with at least one of C₁-C₈-alkyl, (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; preferably in any one of these embodiments Y is a bond. Accordingly, R₁ may be phenyl optionally substituted with C₁-C₈-alkyl. R₁ may be phenyl optionally substituted with(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be phenyl optionally substituted with at least one of F, Cl, I, Br, —NO₂, —N(C₁-C₈-alkyl)H, —NH₂, and/or —N(C₁-C₈-alkyl)₂. R₁ may be phenyl optionally substituted with hydroxy-(C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably, in any one of these embodiments Y is a bond.

In any one of the methods of preparing a conjugate of an antibody molecule, R₁ may represent a 5- or 6-membered heteroaromatic system such as optionally substituted triazolyl or optionally substituted pyridyl. Preferably, in any one of these embodiments Y is a bond.

In any one of the methods of preparing a conjugate of an antibody molecule, R₁ may represent a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; or C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore. Accordingly, R₁ may represent a small molecule, and preferably Y may be O. R₁ may represent C₁-C₈-alkyl, and preferably Y may be O. R₁ may represent C₁-C₈-alkyl substituted with —S—S—(C₁-C₈-alkyl), and preferably Y may be O. R₁ may represent C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₁-C₈-alkyl substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₂-C₈-alkenyl, and preferably Y may be O. R₁ may represent C₁-C₈-alkyl substituted with optionally substituted phenyl, and peferably Y may be O. R₁ may represent C₂-C₈-alkynyl, and preferably Y may be O. R₁ may represent phenyl, and preferably Y may be a bond. R₁ may represent phenyl substituted with —NO₂, and preferably Y may be a bond. R₁ may represent triazolyl substituted with optionally substituted C₁-C₈-alkyl, and preferably Y may be a bond. R₁ may represent triazolyl substituted with a fluorophore, and preferably Y may be a bond.

Preferably, in any one of the methods of preparing a conjugate of an antibody molecule, R₁ may represent C₁-C₈-alkyl. Preferably, R₁ represents methyl, ethyl, propyl or butyl. More preferably, R₁ represents methyl or ethyl. Still more preferably, R₁ represents ethyl. Preferably, in any one of these embodiments R₁ is O.

In any one of the methods of preparing a conjugate of an antibody molecule, R₁ may be selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O. Accordingly, R₁ may be a small molecule. R₁ may be a fluorophore. R₁ may be optionally substituted C₁-C₈-alkyl, preferably methyl, ethyl, propyl or butyl, more preferably methyl or ethyl, still more preferably ethyl. R₁ may be optionally substituted C₂-C₈-alkenyl. R₁ may be optionally substituted optionally substituted C₂-C₈-alkinyl. Preferably, in any one of these embodiments Y is O.

Preferably, in any one of the methods of preparing a conjugate of an antibldy molecule, R₁ is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

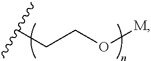

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

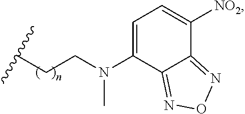

wherein n is 1, 2, 3, 4, 5, 6, 7, 8, 9 or 10, more preferably 4, 5 or 6, still more preferably 5, or more preferably R₁ is

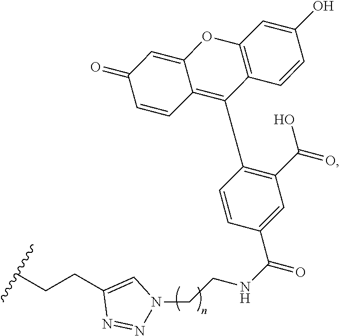

wherein n is 1, 2, 3, 4, 5, 6, 7, 8, 9 or 10, preferably 3, 4 or 5, still more preferably 4; C₂-C₈-alkynyl, preferably

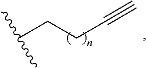

wherein n is 1, 2, 3, 4, or 5, preferably 1, 2 or 3, more preferably 1; or preferably R₁ is

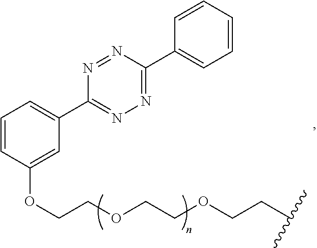

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

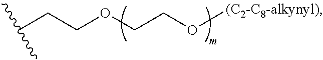

more preferably

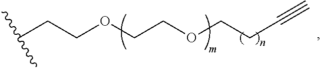

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O. Accordingly, R₁ may be ethyl. R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably R₁ is

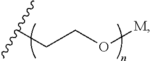

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4. R₁ may be a fluorophore. R₁ may be C₁-C₈-alkyl optionally substituted with a fluorophore. Preferably, R₁ is

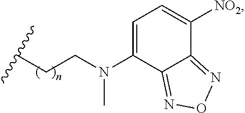

wherein n is 1, 2, 3, 4, 5, 6, 7, 8, 9 or OH 10, more preferably 4, 5 or 6, still more preferably 5. Preferably, R₁ is

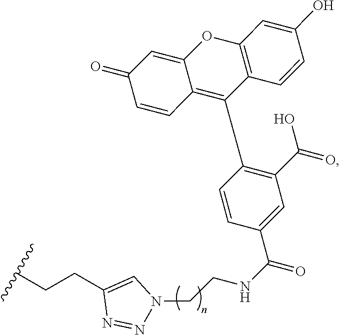

wherein n is 1, 2, 3, 4, 5, 6, 7, 8, 9 or 10, preferably 3, 4 or 5, still more preferably 4. R₁ may be C₂-C₈-alkynyl. Preferably, R₁ is

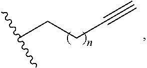

wherein n is 1, 2, 3, 4, or 5, preferably 1, 2 or 3, more preferably 1. Preferably, R₁ is

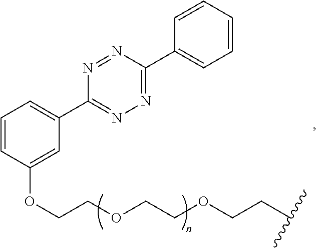

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2. Preferably, R₁ is

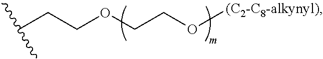

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3. More preferably, R₁ is

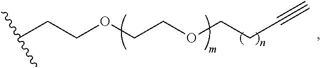

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1. Preferably, in any one of these embodiments Y is O.

Preferably, in any one of the methods of preparing a conjugate of an antibody molecule, R₁ may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

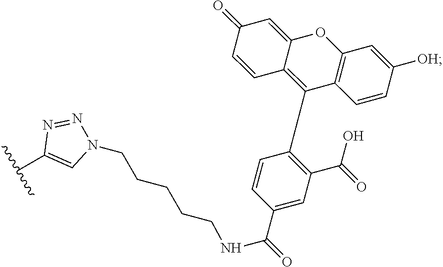 [SMILES: *c1cn(CCCCCNC(=O)c2ccc(-c3c4ccc(=O)cc-4oc4cc(O)ccc34)c(C(=O)O)c2)nn1]

or still more preferably R₁ is

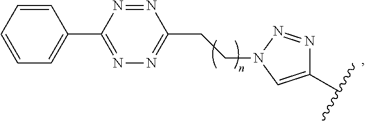

wherein n is 1, 2, 3, 4, 5, 6, 7, 8 or 9, preferably 1, 2 or 3, more preferably 1; or preferably R₁ is

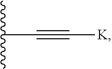

wherein K is H or C₁-C₈-alkyl, preferably K is H; preferably wherein in each instance Y is a bond. Accordingly, R₁ may be optionally substituted aryl. Preferably, R₁ is optionally substituted phenyl. More preferably, R₁ is unsubstituted phenyl. R₁ may be optionally substituted heteroaryl. Preferably, R₁ is optionally substituted triazolyl. More preferably, R₁ is triazolyl substituted with optionally substituted C₁-C₈-alkyl. R₁ may be a fluorophore. More preferably, R₁ is triazolyl substituted with a fluorophore. Still more preferably, R₁ is

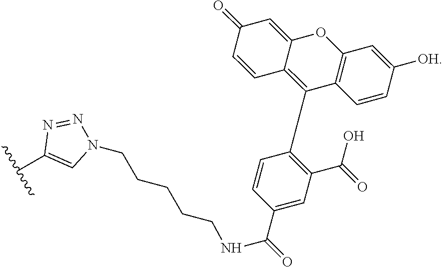 [SMILES: *c1cn(CCCCCNC(=O)c2ccc(-c3c4ccc(=O)cc-4oc4cc(O)ccc34)c(C(=O)O)c2)nn1]

Still more preferably, R₁ is

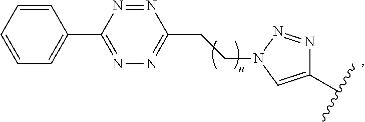

wherein n is 1, 2, 3, 4, 5, 6, 7, 8 or 9, preferably 1, 2 or 3, more preferably 1. Preferably R₁ is

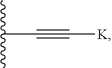

wherein K is H or C₁-C₈-alkyl, preferably H or C₁-C₆-alkyl, more preferably H or C₁-C₄-alkyl, sill more preferably H or C1-C2-alkyl; even more preferably K is H. Preferably, in any one of these embodiments Y is a bond.

In any one of the methods of preparing a conjugate of an antibody molecule, R₁ may represent an amino acid, a peptide, a protein, an antibody, a nucleotide, an oligonucleotide, a saccharide, a polysaccharide, a detectable label, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between R₁ and Y. Preferably, R₁ represents an amino acid. Preferably, R₁ represents a peptide. Preferably, R₁ represents a protein. Preferably, R₁ represents an antibody. Preferably, R₁ represents a nucleotide. Preferably, R₁ represents an oligonucleotide. In some embodiments, R₁ represents a saccharide. In some embodiments, R₁ represents a polysaccharide. In some embodiments, R₁ represents a radioactive or non-radioactive nuclide. In some embodiments, R₁ represents a reporter enzyme. In some embodiments, R₁ represents a protein tag. Preferably, R₁ represents a fluorophore such as CY₅, fluorescein or EDANS. Preferably, R₁ represents biotin. Preferably, R₁ represents a linker. Preferably, R₁ represents a drug. Preferably, R₁ represents a linker-drug conjugate. Preferably, R₁ represents a linker-fluorophore conjugate. In some embodiments R₁ represents a polymer. In some embodiments, R₁ represents a small molecule. In some embodiments, R₁ represents an optionally substituted C₁-C₈-alkyl, preferably an optionally substituted C₁-C₄-alkyl, more preferably an optionally substituted C₁-C₂-alkyl. In some embodiments, R₁ represents an optionally substituted phenyl. Preferably, R₁ represents an optionally substituted aromatic 5- or 6-membered heterocyclic system. Optionally, in any one of these embodiments a linker may be arranged between R₁ and Y.

In any one of the methods of preparing a conjugate of an antibody molecule, R₁ may represent an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between R₁ and Y. Preferably, R₁ represents an amino acid. Preferably, R₁ represents a peptide. Preferably, R₁ represents a protein. Preferably, R₁ represents an antibody. Preferably, R₁ represents a nucleotide. Preferably, R₁ represents an oligonucleotide. In some embodiments, R₁ represents a saccharide. In some embodiments, R₁ represents a polysaccharide. In some embodiments, R₁ represents a radioactive or non-radioactive nuclide. In some embodiments, R₁ represents a reporter enzyme. In some embodiments R₁ represents a polymer. In some embodiments, R₁ represents an optionally substituted C₁-C₈-alkyl, preferably an optionally substituted C₁-C₄-alkyl, more preferably an optionally substituted C₁-C₂-alkyl. In some embodiments, R₁ represents an optionally substituted phenyl. Preferably, R₁ represents an optionally substituted aromatic 5- or 6-membered heterocyclic system. Optionally, in any one of these embodiments a linker may be arranged between R₁ and Y.

Preferably, in any one of the methods of preparing a conjugate of an antibody molecule, R₁ represents an amino acid, a peptide, a protein, an antibody, a nucleotide, or an oligonucleotide; wherein optionally a linker is arranged between R₁ and Y. More preferably, R₁ represents a peptide, a protein, an antibody, or an oligonucleotide; wherein optionally a linker is arranged between R₁ and Y. Preferably, R₁ represents an amino acid. Preferably, R₁ represents a peptide. Preferably, R₁ represents a protein. Preferably, R₁ represents an antibody. Preferably, R₁ represents a nucleotide. Preferably, R₁ represents an oligonucleotide. Optionally, in any one of these embodiments a linker may be arranged between R₁ and Y.

Preferably, in any one of the methods of preparing a conjugate of an antibody molecule, R₁ represents a drug, a protein tag, or a fluorophore such as CY₅, fluorescein or EDANS, biotin, a protein, a peptide, an antibody or an oligonucleotide; wherein optionally a linker is arranged between R₁ and Y. Preferably, R₁ represents a drug. Preferably, R₁ represents a protein tag. Preferably, R₁ represents a linker-drug conjugate. Preferably, R₁ represents a fluorophore such as CY₅, fluorescein or EDANS. Preferably, R₁ represents biotin. Preferably, R₁ represents a protein. Preferably, R₁ represents a peptide. Preferably, R₁ represents an antibody. Preferably, R₁ represents an oligonucleotide. Optionally, in any one of these embodiments a linker may be arranged between R₁ and Y.

Preferably, in any one of the methods of preparing a conjugate of an antibody molecule, R₁ represents a linker, a drug, or a linker-drug conjugate. Preferably, R₁ represents a linker. Preferably, R₁ represents a drug. Preferably, R₁ represents a linker-drug conjugate.

Preferably, in any one of the methods of preparing a conjugate of an antibody molecule, R₁ represents a detectable label. Optionally, in this embodiment, a linker may be arranged between R₁ and Y.

Preferably, in any one of the methods of preparing a conjugate of an antibody molecule, R₁ represents a linker, a fluorophore, or a linker-fluorophore conjugate. Preferably, R₁ represents a linker. Preferably, R₁ represents a fluorophore. Preferably, R₁ represents a linker-fluorophore conjugate.

Preferably, in any one of the methods of preparing a conjugate of an antibody molecule, R₁ represents a small molecule, a fluorophore, a peptide, a protein, or an antibody; wherein optionally a linker is arranged between R₁ and Y. Preferably, R₁ represents a small molecule. Preferably, R₁ represents a fluorophore. Preferably, R₁ represents a peptide. Preferably, R₁ represents a protein. Preferably, R₁ represents an antibody. Optionally, in any one of these embodiments a linker may be arranged between R₁ and Y.

Throughout this specification, wherever it is indicated herein with regard to any method, compound or conjugate, that optionally a linker is arranged between R₁ and Y, or the like, the linker may be virtually any linker known to a person skilled in the art, such as e.g. those linkers disclosed herein for being arranged between ● and Q, for example, a peptidic linker or a straight or branched hydrocarbon-based moiety. The linker can also comprise cyclic moieties. A peptidic linker may comprise, for example, 1 to 50, 1 to 40, 1 to 30, 1 to 20, 1 to 10, 1 to 5, 1 to 3, or 2, or 1 amino acid(s). If the linker is a hydrocarbon-based moiety, the main chain of the linker may comprise only carbon atoms but can also contain heteroatoms such as oxygen (O), nitrogen (N) or sulfur (S) atoms, and/or can contain carbonyl groups (C═O). The linker may be, for example, a C₁-C₂₀ carbon atom chain or a polyether-based chain such as a polyethylene glycol-based chain with —(O—CH₂—CH₂)— repeating units. In typical embodiments of hydrocarbon-based linkers, the linking moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. An illustrative example compound, in which a linker is arranged between R₁ and Y (Y is a fluorophore), is shown in the following:

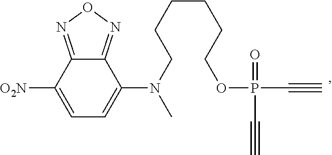 [SMILES: C#CP(=O)(C#C)OCCCCCCN(C)c1ccc([N+](=O)[O-])c2nonc12]

wherein the linker is

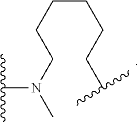 [SMILES: *CCCCCCN(*)C]

The aforementioned exemplary linkers may be also used, for example, when the present specification refers to a “linker” as such, or to a “linker-drug conjugate”, for example in the context of an antibody drug conjugate, or to a “linker-fluorophore conjugate”, for example in the context of an antibody fluorophore conjugate. A person skilled in the art knows to select suitable linkers.

In any one of the methods of preparing a conjugate of an antibody molecule, ▴ may represent Z; and Z represents a residue bound to the phosphorus via a carbon atom and comprising a group ●, wherein ● represents an optionally substituted aliphatic or optionally substituted aromatic residue.

Preferably, in any one of the methods of preparing a conjugate of an antibody, Z is

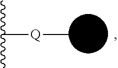

wherein [symbol] indicates the attachment point to the phosphorus and ● is as defined herein; and Q is a moiety comprising at least three main-chain carbon atoms and a carbon-carbon double bond, wherein at least one of the main chain atoms is a heteroatom selected from the group consisting of S, O or N, preferably S. Optionally, in each instance, a linker can be arranged between ● and Q. More preferably, Z is

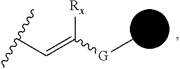

wherein Q is

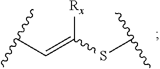

Rₓ is H or C₁-C₈-alkyl; G is S, O or NR₁₀, wherein R₁₀ is H or C₁-C₈-alkyl; and ● is as defined herein; optionally, a linker can be arranged between ● and Q. Preferably, Rₓ is H or C₁-C₆-alkyl, more preferably Rₓ is H or C₁-C₄-alkyl, still more preferably Rₓ is H or C₁-C₂alkyl. Even more preferably, Rₓ is H. Preferably, in any one of the methods, when [symbol] is a triple bond and X is R₃—C, R₃ and Rₓ are the same; more preferably, R₃, Rₓ and R₅ are the same. More preferably, when [symbol] is a triple bond and X is R₃—C, R₃ and Rₓ are both H; even more preferably, R₃, Rₓ and R₅ are each H. Preferably, in any one of the methods, when [symbol] is a double bond and X is

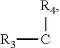

R₃, R₄ and Rₓ are the same; even more preferably, R₃, R₄, Rₓ and R₅ are the same; still more preferably, R₃, R₄, Rₓ, R₅ and V are the same. More preferably, when [symbol] is a double bond and X is

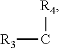

R₃, R₄ and Rₓ are each H; even more preferably, R₃, R₄, Rₓ and R₅ are each H; still more preferably, R₃, R₄, Rₓ, R₅ and V are each H. R₁₀, when present, may be H or C₁-C₆-alkyl, preferably H or C₁-C₄-alkyl, more preferably H or C₁-C₂alkyl. Still more preferably, R₁₀ is H. G may be NR₁₀. G may be O. Preferably, G is S. Accordingly, still more preferably, Z is

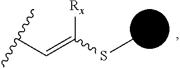

wherein Q is

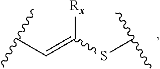

and Rₓ and ● are as defined herein; optionally, a linker can be arranged between ● and Q.

Preferably, in any one of the methods of preparing a conjugate of an antibody, Z is

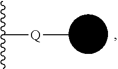

wherein [symbol] indicates the attachment point to the phosphorus and ● is as defined herein; and Q is a five- or six-membered heterocyclic moiety comprising 1, 2 or 3 heteroatoms independently selected from the group consisting of N, O or S. Optionally, in each instance, a linker is arranged between ● and Q. More preferably, Z is selected from the group consisting of

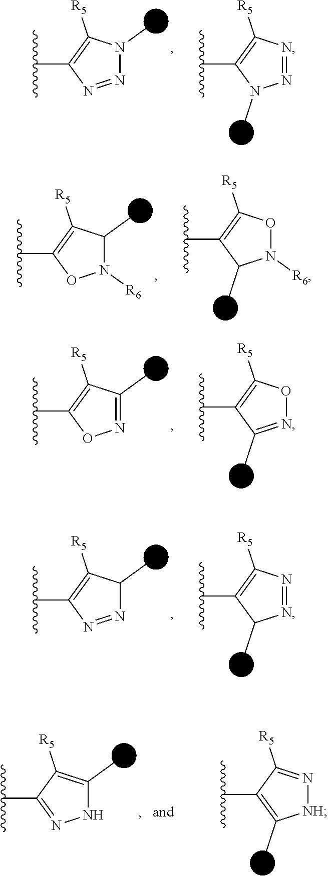

wherein Rₓ is H or C₁-C₈-alkyl; R₆ is C₁-C₈-alkyl, and ● is as defined herein. Accordingly, Z may be

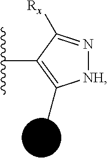

wherein Q is

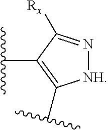

### Z may be

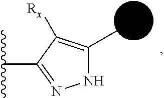

wherein Q is

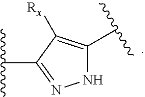

may be

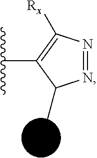

wherein Q is

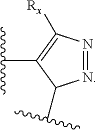

### Z may be

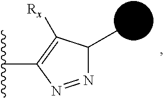

wherein Q is

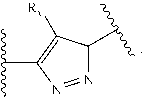

### Z may be

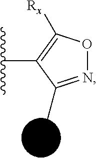

wherein Q is

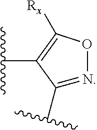

### Z may be

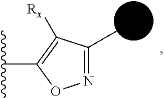

wherein Q is

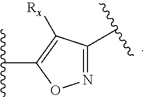

### Z may be

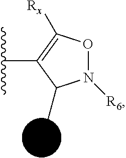

wherein Q is

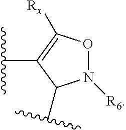

### Z may be

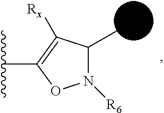

wherein Q is

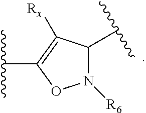

preferably, Z is

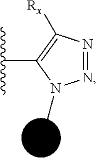

wherein Q is

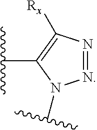

More preferably, Z is

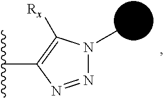

wherein Q is

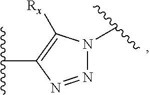

Optionally, in any one of these embodiments a linker can be arranged between ● and Q. Preferably, Rₓ is H or C₁-C₆-alkyl, more preferably H or C₁-C₄-alkyl, still more preferably H or C₁-C₂alkyl. Even more preferably, Rₓ is H. Preferably, in any one of the methods, when [symbol] is a triple bond and X is R₃—C, R₃ and Rₓ are the same; more preferably R₃, Rₓ and R₅ are the same. More preferably, when [symbol] is a triple bond and X is R₃—C, R₃ and Rₓ are both H; even more preferably, R₃, Rₓ and R₅ are each H. Preferably, in any one of the methods, when [symbol] is a double bond and X is

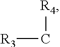

R₃, R₄ and Rₓ are the same; even more preferably, R₃, R₄, Rₓ and R₅ are the same; still more preferably, R₃, R₄, Rₓ, R₅ and V are the same. More preferably, when [symbol] is a double bond and X is

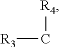

R₃, R₄ and Rₓ are each H; even more preferably, R₃, R₄, Rₓ and R₅ are each H; still more preferably, R₃, R₄, Rₓ, R₅ and V are each H. R₆, when present, may be C₁-C₈-alkyl, preferably C₁-C₆-alkyl, more preferably C₁-C₄-alkyl, still more preferably C₁-C₂alkyl.

Preferably, in any one of the methods of preparing a conjugate of an antibody molecule, Z is

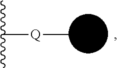

wherein [symbol] indicates the attachment point to the phosphorus and ● is as defined herein; and Q is a moiety comprising a carbon-carbon triple bond bound to the phosphorus in the compound of formula (I), and an optionally substituted phenyl group bound to the carbon-carbon triple bond; or
Q is a moiety comprising a carbon-carbon triple bond bound to the phosphorus in the compound of formula (IV*), and an optionally substituted carbon-carbon double bond bound to the carbon-carbon triple bond. Optionally, in each instance a linker is arranged between ● and Q. More preferably, Z is

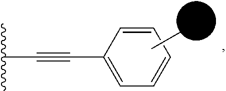

wherein Q is

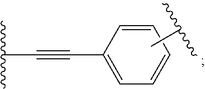

optionally, a linker can be arranged between ● and Q. More preferably, Z is

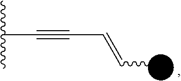

wherein Q is

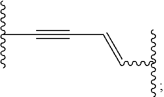

optionally, a linker can be arranged between ● and Q.

In any one of the methods of preparing a conjugate of an antibody molecule, ● may represent an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments, ● represents a protein tag. Preferably, ● represents a fluorophore such as CY₅, fluorescein or EDANS. Preferably, ● represents biotin. Preferably, ● represents a linker. Preferably, ● represents a drug. Preferably, ● represents a linker-drug conjugate. Preferably, ● represents a linker-fluorophore conjugate. In some embodiments ● represents a polymer. In some embodiments, ● represents a small molecule. In some embodiments, ● represents an optionally substituted C₁-C₈-alkyl, preferably an optionally substituted C₁-C₄-alkyl, more preferably an optionally substituted C₁-C₂-alkyl. In some embodiments, represents an optionally substituted phenyl. Preferably, ● represents an optionally substituted aromatic 5- or 6-membered heterocyclic system. Optionally, in any one of these embodiments a linker may be arranged between ● and Q.

In any one of the methods of preparing a conjugate of an antibody molecule, ● may represent an amino acid, a peptide, a protein, an antibody, a nucleotide, an oligonucleotide, a saccharide, a polysaccharide, a detectable label, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments ● represents a polymer. In some embodiments, ● represents an optionally substituted C₁-C₈-alkyl, preferably an optionally substituted C₁-C₄-alkyl, more preferably an optionally substituted C₁-C₂-alkyl. In some embodiments, ● represents an optionally substituted phenyl. Preferably, ● represents an optionally substituted aromatic 5- or 6-membered heterocyclic system. Optionally, in any one of these embodiments a linker may be arranged between ● and Q.

Preferably, in any one of the methods of preparing a conjugate of an antibody molecule, ● represents an amino acid, a peptide, a protein, an antibody, a nucleotide, or an oligonucleotide; wherein optionally a linker is arranged between ● and Q. More preferably, ● represents a peptide, a protein, an antibody, or an oligonucleotide; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. Optionally, in any one of these embodiments a linker may be arranged between ● and Q.

Preferably, in any one of the processes of preparing a conjugate of an antibody molecule, ● represents a drug, a protein tag, or a fluorophore such as CY₅, fluorescein or EDANS, biotin, a protein, a peptide, an antibody or an oligonucleotide; wherein optionally a linker is arranged between ● and Q. Preferably, ● represents a drug. Preferably, ● represents a protein tag. Preferably, ● represents a linker-drug conjugate. Preferably, ● represents a fluorophore such as CY₅, fluorescein or EDANS. Preferably, ● represents biotin. Preferably, ● represents a protein. Preferably, ● represents a peptide. Preferably, ● represents an antibody. Preferably, ● represents an oligonucleotide. Optionally, in any one of these embodiments a linker may be arranged between ● and Q.

Preferably, in any one of the methods of preparing a conjugate of an antibody molecule, ● represents a linker, a drug, or a linker-drug conjugate. Preferably, ● represents a linker. Preferably, ● represents a drug. Preferably, ● represents a linker-drug conjugate.

Preferably, in any one of the methods of preparing a conjugate of an antibody molecule, ● represents a detectable label. Optionally, in this embodiment, a linker may be arranged between ● and Q.

Preferably, in any one of the methods of preparing a conjugate of an antibody molecule, ● represents a linker, a fluorophore, or a linker-fluorophore conjugate. Preferably, ● represents a linker. Preferably, ● represents a fluorophore. Preferably, ● represents a linker-fluorophore conjugate.

Preferably, in any one of the methods of preparing a conjugate of an antibody molecule, ● represents a small molecule, a fluorophore, a peptide, a protein, or an antibody; wherein optionally a linker is arranged between ● and Q. Preferably, ● represents a small molecule. Preferably, ● represents a fluorophore. Preferably, ● represents a peptide. Preferably, ● represents a protein. Preferably, ● represents an antibody. Optionally, in any one of these embodiments a linker may be arranged between ● and Q.

In any one of the methods of preparing a conjugate of an antibody molecule, ● may represent a small molecule; C₁-C₈-alkyl optionally substituted with at least one of (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; wherein optionally a linker is arranged between ● and Q. Accordingly, ● may be a small molecule. ● may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be C₁-C₈-alkyl optionally substituted with at least one of F, Cl, Br, I, —NO₂, —N(C₁-C₈-alkyl)H, —NH₂, —N₃, —N(C₁-C₈-alkyl)₂, ═O, C₃-C₈-cycloalkyl, and/or —S—S—(C₁-C₈-alkyl). ● may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be C₁-C₈-alkyl optionally substituted with C₂-C₈-alkenyl. ● may be C₁-C₈-alkyl optionally substituted with C₂-C₈-alkynyl. Optionally, in any one of these embodiments a linker may be arranged between ● and Q.

In any one of the methods of preparing a conjugate of an antibody molecule, ● may represent phenyl optionally independently substituted with at least one of C₁-C₈-alkyl, (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; wherein optionally a linker is arranged between ● and Q. Accordingly, ● may be phenyl optionally substituted with C₁-C₈-alkyl. ● may be phenyl optionally substituted with(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be phenyl optionally substituted with at least one of F, Cl, I, Br, —NO₂, —N(C₁-C₈-alkyl)H, —NH₂, and/or —N(C₁-C₈-alkyl)₂. ● may be phenyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Optionally, in any one of these embodiments a linker may be arranged between ● and Q.

In any one of the methods of preparing a conjugate of an antibody molecule, R₁ may represent a 5- or 6-membered heteroaromatic system such as optionally substituted triazolyl or optionally substituted pyridyl. Optionally, in any one of these embodiments a linker may be arranged between ● and Q.

In any one of the methods of preparing a conjugate of an antibody molecule, ● may represent a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; or C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore; wherein optionally a linker is arranged between ● and Q. Accordingly, ● may represent a small molecule. ● may represent C₁-C₈-alkyl. ● may represent C₁-C₈-alkyl substituted with —S—S—(C₁-C₈-alkyl). ● may represent C₁-C₈-alkyl substituted with (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may represent C₁-C₈-alkyl substituted with hydroxy-(C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may represent C₂-C₈-alkenyl. ● may represent C₁-C₈-alkyl substituted with optionally substituted phenyl. ● may represent phenyl. ● may represent phenyl substituted with —NO₂. ● may represent triazolyl substituted with optionally substituted C₁-C₈-alkyl. ● may represent triazolyl substituted with a fluorophore. Optionally, in any one of these embodiments a linker may be arranged between ● and Q.

Preferably, in any one of the methods of preparing a conjugate of an antibody molecule, ● may represent C₁-C₈-alkyl; wherein optionally a linker is arranged between ● and Q. Preferably, ● represents methyl, ethyl, propyl or butyl. More preferably, ● represents methyl or ethyl. Still more preferably, ● represents ethyl. Preferably, in any one of these embodiments R₁ is O. Optionally, in any one of these embodiments a linker may be arranged between ● and Q.

In any one of the methods of preparing a conjugate of an antibody molecule, ● may be selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; wherein optionally a linker is arranged between ● and Q. Accordingly, ● may be a small molecule. ● may be a fluorophore. ● may be optionally substituted C₁-C₈-alkyl, preferably methyl, ethyl, propyl or butyl, more preferably methyl or ethyl, still more preferably ethyl. ● may be optionally substituted C₂-C₈-alkenyl. ● may be optionally substituted optionally substituted C₂-C₈-alkinyl. Optionally, in any one of these embodiments a linker may be arranged between ● and Q.

Preferably, in any one of the methods of preparing a conjugate of an antibody molecule, ● is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably ● is

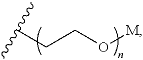

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably ● is

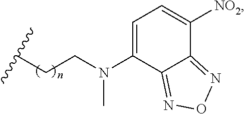

wherein n is 1, 2, 3, 4, 5, 6, 7, 8, 9 or 10, more preferably 4, 5 or 6, still more preferably 5, or more preferably ● is

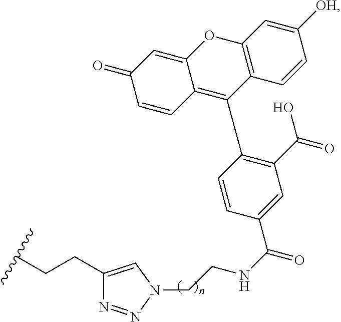

wherein n is 1, 2, 3, 4, 5, 6, 7, 8, 9 or 10, preferably 3, 4 or 5, still more preferably 4; C₂-C₈-alkynyl, preferably

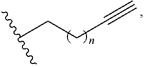

wherein n is 1, 2, 3, 4, or 5, preferably 1, 2 or 3, more preferably 1; or preferably ● is

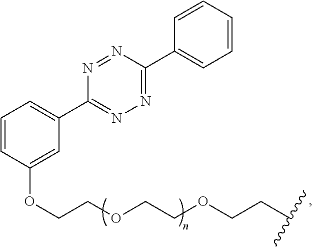

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably ● is

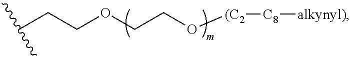

more preferably

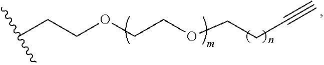

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; wherein optionally a linker is arranged between ● and Q. Accordingly, ● may be ethyl. ● may be C₁-C₈alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably ● is

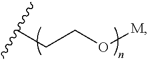

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4. ● may be a fluorophore. ● may be C₁-C₈alkyl optionally substituted with a fluorophore. Preferably, ● is

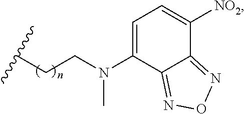

wherein n is 1, 2, 3, 4, 5, 6, 7, 8, 9 or 10, more preferably 4, 5 or 6, still more preferably 5. Preferably, ● is

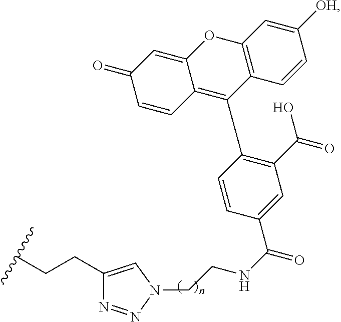

wherein n is 1, 2, 3, 4, 5, 6, 7, 8, 9 or 10, preferably 3, 4 or 5, still more preferably 4. R₁ may be C₂-C₈-alkynyl. Preferably, ● is

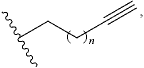

wherein n is 1, 2, 3, 4, or 5, preferably 1, 2 or 3, more preferably 1. Preferably, ● is

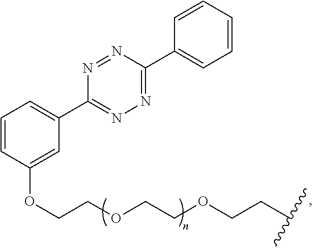

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2. Preferably, ● is

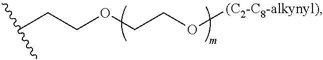

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3. More preferably, ● is

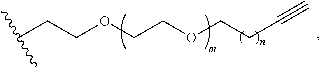

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1. Optionally, in any one of these embodiments a linker may be arranged between ● and Q.

Preferably, in any one of the methods of preparing a conjugate of an antibody molecule, ● may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably ● is

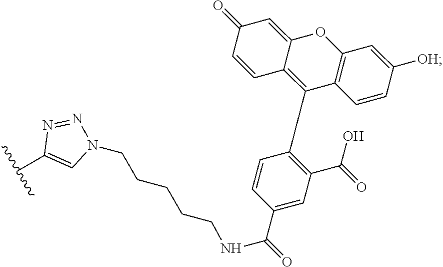 [SMILES: *c1cn(CCCCCNC(=O)c2ccc(-c3c4ccc(=O)cc-4oc4cc(O)ccc34)c(C(=O)O)c2)nn1]

or still more preferably ● is

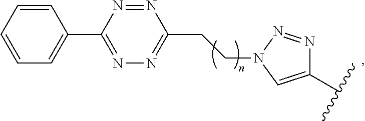

wherein n is 1, 2, 3, 4, 5, 6, 7, 8 or 9, preferably 1, 2 or 3, more preferably 1; or preferably ● is

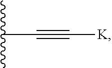

wherein K is H or C₁-C₈-alkyl, preferably K is H; wherein optionally a linker is arranged between ● and Q. Accordingly, ● may be optionally substituted aryl. Preferably, ● is optionally substituted phenyl. More preferably, ● is unsubstituted phenyl. ● may be optionally substituted heteroaryl. Preferably, ● is optionally substituted triazolyl. More preferably, ● is triazolyl substituted with optionally substituted C₁-C₈-alkyl. ● may be a fluorophore. More preferably, S is triazolyl substituted with a fluorophore. Still more preferably, ● is

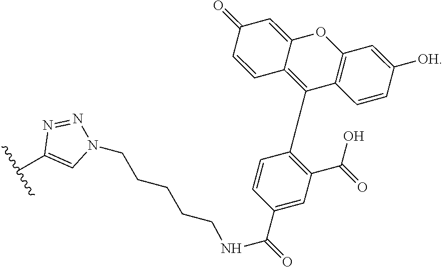 [SMILES: *c1cn(CCCCCNC(=O)c2ccc(-c3c4ccc(=O)cc-4oc4cc(O)ccc34)c(C(=O)O)c2)nn1]

Still more preferably, ● is

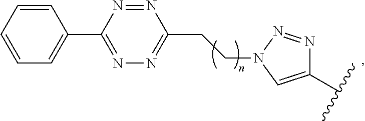

wherein n is 1, 2, 3, 4, 5, 6, 7, 8 or 9, preferably 1, 2 or 3, more preferably 1. Preferably ● is

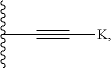

wherein K is H or C₁-C₈-alkyl, preferably H or C₁-C₆-alkyl, more preferably H or C₁-C₄-alkyl, still more preferably H or C₁-C₂-alkyl; even more preferably K is H. Optionally, in any one of these embodiments a linker may be arranged between ● and Q.

Throughout this specification, wherever it is indicated herein with regard to any method, compound or conjugate, that optionally a linker is arranged between ● and Q, or the like, the linker may be virtually any linker known to a person skilled in the art, for example, a peptidic linker or a straight or branched hydrocarbon-based moiety, e.g. any one of those linkers described herein. The linker can also comprise cyclic moieties. A peptidic linker may comprise, for example, 1 to 50, 1 to 40, 1 to 30, 1 to 20, 1 to 10, 1 to 5, 1 to 3, or 2, or 1 amino acid(s). If the linker is a hydrocarbon-based moiety, the main chain of the linker may comprise only carbon atoms but can also contain heteroatoms such as oxygen (O), nitrogen (N) or sulfur (S) atoms, and/or can contain carbonyl groups (C═O). The linker may be, for example, a C₁-C₂₀ carbon atom chain or a polyether-based chain such as a polyethylene glycol-based chain with —(O—CH₂—CH₂)— repeating units. In typical embodiments of hydrocarbon-based linkers, the linking moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. A further illustrative example compound, in which a linker is arranged between ● and Q, is shown in the following:

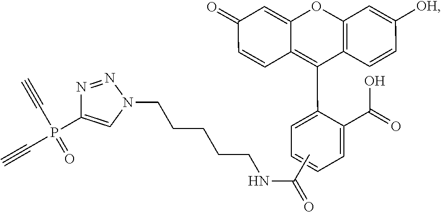

wherein the linker is

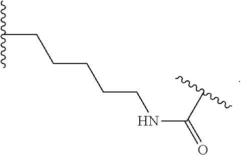 [SMILES: *CCCCCNC(*)=O]

The aforementioned exemplary linkers may be also used, for example, when the present specification refers to a “linker” as such, or to a “linker-drug conjugate”, for example in the context of an antibody drug conjugate, or to a “linker-fluorophore conjugate”, for example in the context of an antibody fluorophore conjugate. A person skilled in the art knows to select suitable linkers.

### Compounds

### Compounds of Formula (I), (IV), (III), (L) and (IIIa)

The present invention also relates to a compound of formula (I)

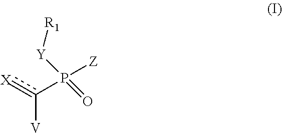

wherein
[symbol] represents a triple bond or a double bond;
V is absent when [symbol] is a triple bond; or
V represents H or C₁-C₈-alkyl when is a double bond;
X represents R₃—C when is a triple bond; or
X represents

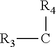

when [symbol] is a double bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl; and
Z represents a residue bound to the phosphorus via a carbon atom and comprising a group ●, wherein ● represents an optionally substituted aliphatic or optionally substituted aromatic residue.

In any one of the compounds of formula (I), the [symbol] V, X, Y, R₁, R², R₃, R₄, Z, and ● may be as defined herein for any one of the methods, compounds and/or conjugates. Any [symbol] V, X, Y, R₁, R², R₃, R₄, Z, and ● as defined herein for any one of the methods, compounds and/or conjugates may be combined with each other.

Preferably, in any one of the compounds of formula (I), [symbol] represents a triple bond; V is absent; X represents R₃—C, and R₃ represents H or C₁-C₈-alkyl. Preferably, R₃ represents H or C₁-C₆-alkyl, more preferably H or C₁-C₄-alkyl, still more preferably H or C₁-C₂-alkyl. Even more preferably, R₃ is H.

In some embodiments, in any one of the compounds of formula (I), [symbol] may represent a double bond; V may be H or C₁-C₈-alkyl; X may represent

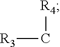

and R₃ and R₄ may independently represent H or C₁-C₈-alkyl. Preferably, R₃ and R₄ independently represent H or C₁-C₆-alkyl, more preferably H or C₁-C₄-alkyl, still more preferably H or C₁-C₂-alkyl. Preferably, R₃ and R₄ are the same; even more preferably, R₃, R₄ and V are the same. More preferably, R₃ and R₄ are both H. Preferably, V is H or C₁-C₆-alkyl, more preferably H or C₁-C₄-alkyl, still more preferably H or C₁-C₂-alkyl. Even more preferably, V is H. In preferred embodiments, R₃, R₄ and V are each H.

The present invention also relates to a compound of formula (III)

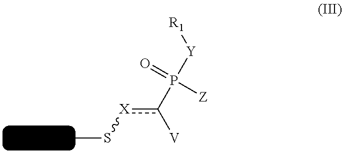

wherein
[symbol] represents a double bond; or
[symbol] represents a bond;
V is absent when [symbol] is a double bond; or
V represents H or C₁-C₈-alkyl when [symbol] is a bond;
X represents R₃—C when [symbol] is a double bond; or
X represents

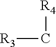

when [symbol] is a bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
Z represents a residue bound to the phosphorus via a carbon atom and comprising a group ●, wherein ● represents an optionally substituted aliphatic or optionally substituted aromatic residue; and [symbol] represents an amino acid, a peptide, a protein, an antibody, a nucleotide, an olignucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system.

In any one of the compounds of formula (III), the [symbol] V, X, Y, R₁, R², R₃, R₄, Z, ●, and [symbol] may be as defined herein for any one of the methods, compounds and/or conjugates. Any [symbol] , V, X, Y, R₁, R², R₃, R₄, Z, ●, and [symbol] defined herein for any one of the methods, compounds and/or conjugates may be combined with each other.

Preferably, in any one of the compounds of formula (III), [symbol] represents a double bond; V is absent; X represents R₃—C, and R₃ represents H or C₁-C₈-alkyl. Preferably, R₃ represents H or C₁-C₆-alkyl, more preferably H or C₁-C₄-alkyl, still more preferably H or C₁-C₂-alkyl. Even more preferably, R₃ is H.

In some embodiments, in any one of the compounds of formula (III), [symbol] may represent a bond; V may be H or C₁-C₈-alkyl; X may represent

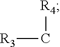

and R₃ and R₄ may independently represent H or C₁-C₈-alkyl. Preferably, R₃ and R₄ independently represent H or C₁-C₆alkyl, more preferably H or C₁-C₄-alkyl, still more preferably H or C₁-C₂alkyl. Preferably, R₃ and R₄ are the same; even more preferably, R₃, R₄ and V are the same. More preferably, R₃ and R₄ are both H. Preferably, V is H or C₁-C₆-alkyl, more preferably H or C₁-C₄-alkyl, still more preferably H or C₁-C₂-alkyl. Even more preferably, V is H. In preferred embodiments, R₃, R₄ and V are each H.

Preferably, in any one of the compounds of formula (I) or (III), Z is

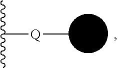

wherein [symbol] indicates the attachment point to the phosphorus and ● is as defined herein; and Q is a moiety comprising at least three main-chain carbon atoms and a carbon-carbon double bond, wherein at least one of the main chain atoms is a heteroatom selected from the group consisting of S, O or N, preferably S. Optionally, in each instance, a linker can be arranged between ● and Q. More preferably, Z is

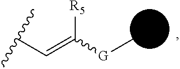

wherein Q is

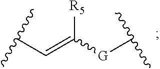

R₅ is H or C₁-C₈-alkyl; G is S, O or NR₁₀, wherein R₁₀ is H or C₁-C₈-alkyl; and ● is as defined herein; optionally, a linker can be arranged between ● and Q. Preferably, R₅ is H or C₁-C₆-alkyl, more preferably R₅ is H or C₁-C₄-alkyl, still more preferably R₅ is H or C₁-C₂-alkyl. Even more preferably, R₅ is H. Preferably, in any one of the compounds of formula (I) or (III), when X is R₃—C, R₃ and R₅ are the same; more preferably, when X is R₃—C, R₃ and R₅ are both H. Preferably, in any one of the compounds of formula (I) or (III), when X is

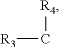

R₃, R₄ and R₅ are the same; even more preferably, R₃, R₄, R₅ and V are the same. More preferably, when X is

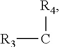

R₃, R₄ and R₅ are each H; more preferably, R₃, R₄, R₅ and V are each H. R₁₀, when present, may be H or C₁-C₆-alkyl, preferably H or C₁-C₄-alkyl, more preferably H or C₁-C₂-alkyl. Still more preferably, R₁₀ is H. G may be NR₁₀. G may be O. Preferably, G is S. Accordingly, preferably, Z is

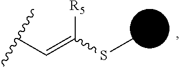

wherein Q is

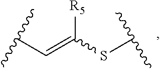

and R₅ and ● are as defined herein; optionally, a linker can be arranged between ● and Q.

Preferably, in any one of the compounds of formula (I) or (III), Z is

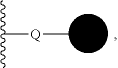

wherein [symbol] indicates the attachment point to the phosphorus and ● is as defined herein; and Q is a five- or six-membered heterocyclic moiety comprising 1, 2 or 3 heteroatoms independently selected from the group consisting of N, O or S. Optionally, in each instance, a linker is arranged between ● and Q. More preferably, Z is selected from the group consisting

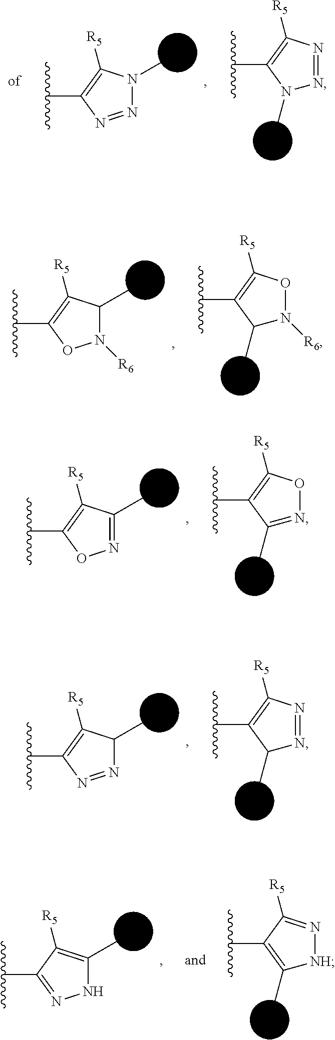

wherein R₅ is H or C₁-C₈-alkyl; R₆ is C₁-C₈-alkyl, and ● is as defined herein. Accordingly, Z may be

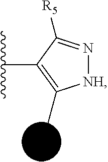

wherein Q is

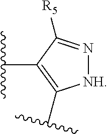

### Z may be

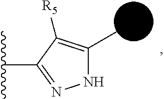

wherein Q is

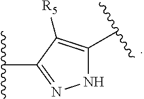

### Z may be,

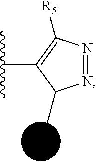

wherein Q is

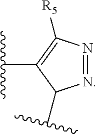

### Z may be

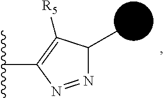

wherein Q is

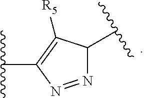

### Z may be

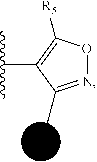

wherein Q is

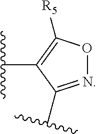

### Z may be

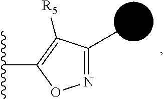

wherein Q is

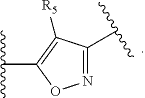

### Z may be

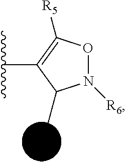

wherein Q is

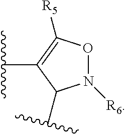

### Z may be

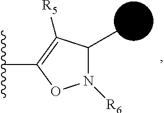

wherein Q is

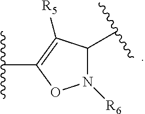

### Preferably, Z is

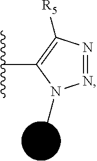

wherein Q is

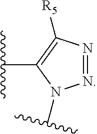

More preferably, Z is

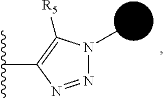

wherein Q is

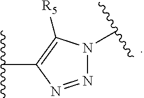

Optionally, in any one of these embodiments a linker can be arranged between ● and Q. Preferably, R₅ is H or C₁-C₆-alkyl, more preferably H or C₁-C₄-alkyl, still more preferably H or C₁-C₂alkyl. Even more preferably, R₅ is H. Preferably, in any one of the compounds of formula (I) or (III), when X is R₃—C, R₃ and R₅ are the same; more preferably, when X is R₃—C, R₃ and R₅ are both H. Preferably, in any one of the compounds of formula (I) or (III), when X is

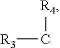

R₃, R₄ and R₅ are the same; even more preferably, R₃, R₄, R₅ and V are the same. More preferably, when X is

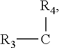

R₃, R₄ and R₅ are each H; even more preferably, R₃, R₄, R₅ and V are H. R₆, when present, may be C₁-C₈-alkyl, preferably C₁-C₆-alkyl, more preferably C₁-C₄-alkyl, still more preferably C₁-C₂alkyl.

Preferably, in any one of the compounds of formula (I) or (III), Z is

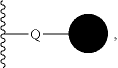

wherein [symbol] indicates the attachment point to the phosphorus and ● is as defined herein; and
Q is a moiety comprising a carbon-carbon triple bond bound to the phosphorus in the compound of formula (I) or formula (III), and an optionally substituted phenyl group bound to the carbon-carbon triple bond; or
Q is a moiety comprising a carbon-carbon triple bond bound to the phosphorus in the compound of formula (I) or formula (II), and an optionally substituted carbon-carbon double bond bound to the carbon-carbon triple bond. Optionally, in each instance a linker is arranged between ● and Q. More preferably, Z is

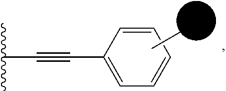

wherein Q is

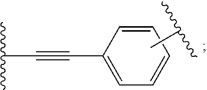

optionally, a linker can be arranged between ● and Q. More preferably, Z is

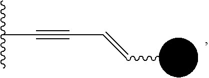

wherein Q is

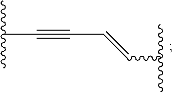

optionally, a linker can be arranged between ● and Q.

In any one of the compounds of formula (I) or (III), Y represents O, NR² wherein R² represents H or C₁-C₈-alkyl, S, or a bond. Preferably, in each instance, R₁ is bound to Y via a carbon atom.

Accordingly, Y may be O (oxygen).

Y may be NR². R² is H or C₁-C₈-alkyl. Preferably, R₂ is C₁-C₈-alkyl. More preferably, R² is methyl, ethyl, propyl or butyl. Still more preferably, R² is methyl or ethyl.

Y may be S (sulfur).

Y may be a bond. In particular, Y may be a single bond which connects R₁ with the phosphorus.

In any one of the compounds of formula (I) or (III), R₁ may represent a small molecule; C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; preferably in any one of these embodiments Y is O. Accordingly, R₁ may be a small molecule. R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with at least one of F, Cl, Br, I, —NO₂, —N(C₁-C₈-alkyl)H, —NH₂, —N₃, —N(C₁-C₈-alkyl)₂, ═O, C₃-C₈-cycloalkyl, and/or —S—S—(C₁-C₈-alkyl). R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with C₂-C₈-alkenyl. R₁ may be C₁-C₈-alkyl optionally substituted with C₂-C₈-alkynyl. Preferably, in any one of these embodiments Y is O.

In any one of the compounds of formula (I) or (III), R₁ may represent phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; preferably in each instance Y is a bond. Accordingly, R₁ may be phenyl optionally substituted with C₁-C₈-alkyl. R₁ may be phenyl optionally substituted with(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be phenyl optionally substituted with at least one of F, Cl, I, Br, —NO₂, —N(C₁-C₈-alkyl)H, —NH₂, and/or —N(C₁-C₈-alkyl)₂. R₁ may be phenyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably, in any one of these embodiments Y is a bond.

In any one of the compounds of formula (I) or (III), R₁ may represent a 5- or 6-membered heteroaromatic system such as optionally substituted triazolyl or optionally substituted pyridyl. Preferably, in any one of these embodiments Y is a bond.

In any one of the compounds of formula (I) or (III), R₁ may represent a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; or C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore. Accordingly, R₁ may represent a small molecule, and preferably Y may be O. R₁ may represent C₁-C₈-alkyl, and preferably Y may be O. R₁ may represent C₁-C₈-alkyl substituted with —S—S—(C₁-C₈-alkyl), and preferably Y may be O. R₁ may represent C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₁-C₈-alkyl substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₂-C₈-alkenyl, and preferably Y may be O. R₁ may represent C₁-C₈-alkyl substituted with optionally substituted phenyl, and peferably Y may be O. R₁ may represent C₂-C₈-alkynyl, and preferably Y may be O. R₁ may represent phenyl, and preferably Y may be a bond. R₁ may represent phenyl substituted with —NO₂, and preferably Y may be a bond. R₁ may represent triazolyl substituted with optionally substituted C₁-C₈-alkyl, and preferably Y may be a bond. R₁ may represent triazolyl substituted with a fluorophore, and preferably Y may be a bond.

Preferably, in any one of the compounds of formula (I) or (III), R₁ may represent C₁-C₈-alkyl. Preferably, R₁ represents methyl, ethyl, propyl or butyl. More preferably, R₁ represents methyl or ethyl. Still more preferably, R₁ represents ethyl. Preferably, in any one of these embodiments R₁ is O.

In any one of the compounds of formula (I) or (III), R₁ may be selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O. Accordingly, R₁ may be a small molecule. R₁ may be a fluorophore. R₁ may be optionally substituted C₁-C₈-alkyl, preferably methyl, ethyl, propyl or butyl, more preferably methyl or ethyl, still more preferably ethyl. R₁ may be optionally substituted C₂-C₈-alkenyl. R₁ may be optionally substituted optionally substituted C₂-C₈-alkinyl. Preferably, in any one of these embodiments Y is O.

Preferably, in any one of the compounds of formula (I) or (III), R₁ is selected from the group consisting of ethyl; C₁-C₈-alkyl optionally substituted with (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₁-C₈-alkyl optionally substituted with hydroxy-(C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

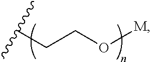

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

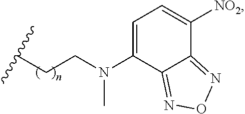

wherein n is 1, 2, 3, 4, 5, 6, 7, 8, 9 or 10, more preferably 4, 5 or 6, still more preferably 5, or more preferably R₁ is

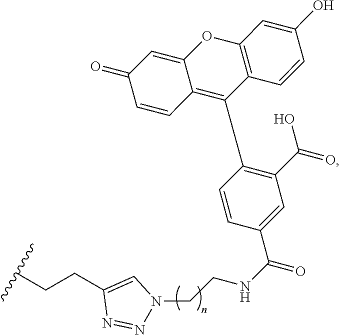

wherein n is 1, 2, 3, 4, 5, 6, 7, 8, 9 or 10, preferably 3, 4 or 5, still more preferably 4; C₂-C₈-alkynyl, preferably

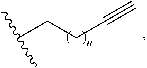

wherein n is 1, 2, 3, 4, or 5, preferably 1, 2 or 3, more preferably 1; or preferably R₁ is

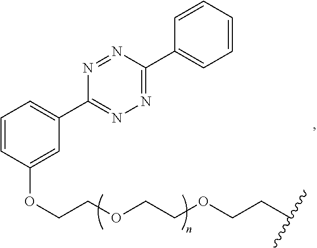

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

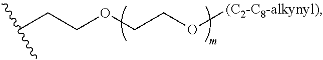

more preferably

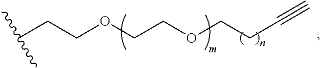

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O. Accordingly, R₁ may be ethyl. R₁ may be C₁-C₈alkyl optionally substituted with (C₁-C₈alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably R₁ is

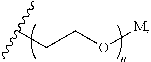

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4. R₁ may be a fluorophore. R₁ may be C₁-C₈-alkyl optionally substituted with a fluorophore. Preferably, R₁ is

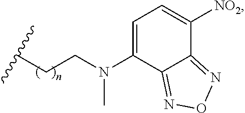

wherein n is 1, 2, 3, 4, 5, 6, 7, 8, 9 or 10, more preferably 4, 5 or 6, still more preferably 5. Preferably, R₁ is

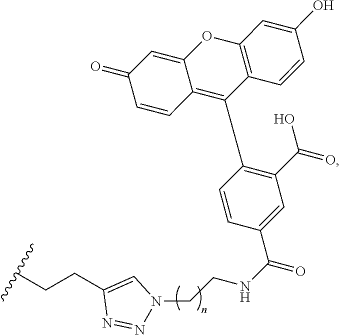

wherein n is 1, 2, 3, 4, 5, 6, 7, 8, 9 or 10, preferably 3, 4 or 5, still more preferably 4. R₁ may be C₂-C₈-alkynyl. Preferably, R₁ is

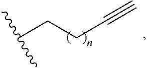

wherein n is 1, 2, 3, 4, or 5, preferably 1, 2 or 3, more preferably 1. Preferably, R₁ is

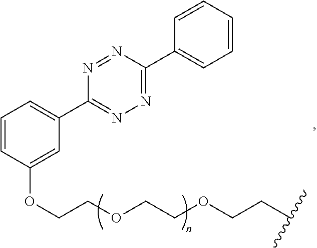

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2. Preferably, R₁ is

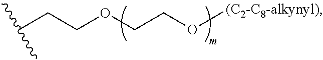

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3. More preferably, R₁ is

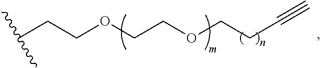

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1. Preferably, in any one of these embodiments Y is O.

In any one of the compounds of formula (I) or (III), R₁ may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

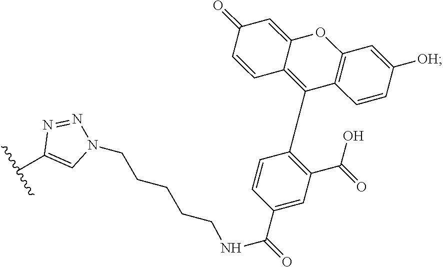 [SMILES: *c1cn(CCCCCNC(=O)c2ccc(-c3c4ccc(=O)cc-4oc4cc(O)ccc34)c(C(=O)O)c2)nn1]

or still more preferably R₁ is

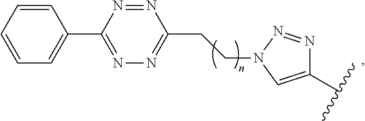

wherein n is 1, 2, 3, 4, 5, 6, 7, 8 or 9, preferably 1, 2 or 3, more preferably 1; or preferably R₁ is

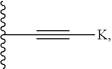

wherein K is H or C₁-C₈-alkyl, preferably K is H; preferably wherein in each instance Y is a bond. Accordingly, R₁ may be optionally substituted aryl. Preferably, R₁ is optionally substituted phenyl. More preferably, R₁ is unsubstituted phenyl. R₁ may be optionally substituted heteroaryl. Preferably, R₁ is optionally substituted triazolyl. More preferably, R₁ is triazolyl substituted with optionally substituted C₁-C₈-alkyl. R₁ may be a fluorophore. More preferably, R₁ is triazolyl substituted with a fluorophore. Still more preferably, R₁ is

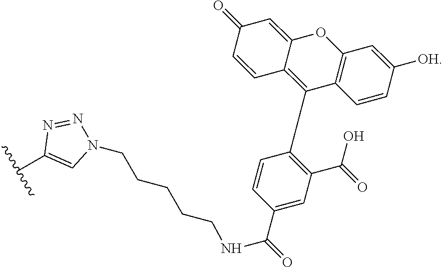 [SMILES: *c1cn(CCCCCNC(=O)c2ccc(-c3c4ccc(=O)cc-4oc4cc(O)ccc34)c(C(=O)O)c2)nn1]

Still more preferably, R₁ is

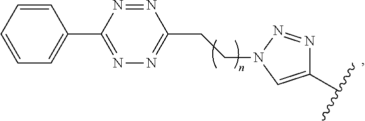

wherein n is 1, 2, 3, 4, 5, 6, 7, 8 or 9, preferably 1, 2 or 3, more preferably 1. Preferably R₁ is

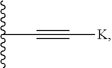

wherein K is H or C₁-C₈-alkyl, preferably H or C₁-C₆-alkyl, more preferably H or C₁-C₄-alkyl, sill more preferably H or C1-C2-alkyl; even more preferably K is H. Preferably, in any one of these embodiments Y is a bond.

In any one of the compounds of formula (I) or (III), R₁ may be C₁-C₈-alkyl, preferably methyl, ethyl, propyl or butyl; more preferably methyl or ethyl; still more preferably ethyl; and Z may be

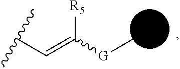

wherein Q is

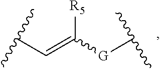

R₅ is as defined herein, preferably R₅ is H; G is S, O or NR₁₀, wherein R₁₀ is as defined herein, preferably R₁₀ is H; and ● is as defined herein. Preferably, Z is

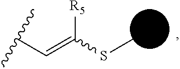

wherein Q is

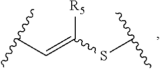

R₅ is as defined herein, preferably R₅ is H; and ● is as defined herein. Preferably, in any one of these embodiments Y is O. Optionally, in any one of these embodiments, R₁ may be C₁-C₈-alkyl substituted with a fluorophore. Optionally, in any one of these embodiments, a linker may be arranged between ● and Q.

In an one of the compounds of formula (I) or (III), R₁ may be C₂-C₈-alkynyl, preferably

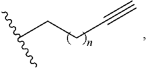

wherein n is 1, 2, 3, 4, or 5, preferably 1, 2 or 3, more preferably 1; and Z may be

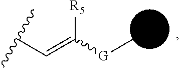

wherein Q is

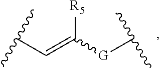

R₅ is as defined herein, preferably R₅ is H; G is S, O or NR₁₀, wherein R₁₀ is as defined herein, preferably R₁₀ is H; and ● is as defined herein; preferably, Z is

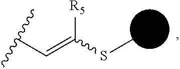

wherein Q is

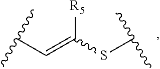

R₅ is as defined herein, preferably R₅ is H, and ● is as defined herein. Preferably, in any one of these embodiments Y is O. Optionally, in any one of these embodiments, a linker may be arranged between ● and Q.

In any one of the compounds of formula (I) or (III), R₁ may be

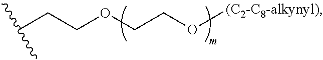

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3; preferably R₁ may be

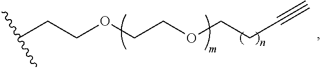

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and Z may be

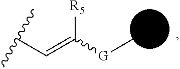

wherein Q is

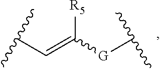

R₅ is as defined herein, preferably R₅ is H; G is S, O or NR₁₀, wherein R₁₀ is as defined herein, preferably R₁₀ is H; and ● is as defined herein. Preferably, Z is

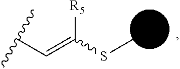

wherein Q is

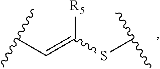

R₅ is as defined herein, preferably R₅ is H, and ● is as defined herein. Preferably, in any one of these embodiments Y is O. Optionally, in any one of these embodiments, a linker may be arranged between ● and Q.

In any one of the compounds of formula (I) or (III), R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be

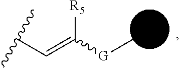

wherein Q is

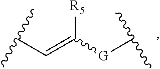

R₅ is as defined herein, preferably R₅ is H; G is S, O or NR₁₀, wherein R₁₀ is as defined herein, preferably R₁₀ is H; and ● is as defined herein. Preferably, Z is

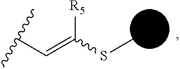

wherein Q is

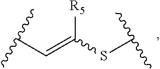

R₅ is as defined herein, preferably R₅ is H, and ● is as defined herein. Preferably, in any one of these embodiments Y is O. Optionally, in any one of these embodiments, a linker may be arranged between ● and Q.

In any one of the compounds of formula (I) or (III), R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be

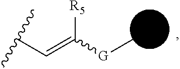

wherein Q is

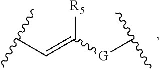

R₅ is as defined herein, preferably R₅ is H; G is S, O or NR₁₀, wherein R₁₀ is as defined herein, preferably R₁₀ is H; and ● is as defined herein. Preferably, Z is

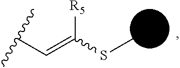

wherein Q is

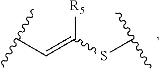

R₅ is as defined herein, preferably R₅ is H, and ● is as defined herein. Preferably, in any one of these embodiments Y is O. Optionally, in any one of these embodiments, a linker may be arranged between ● and Q.

Preferably, in any one of the compounds of formula (I) or (III), R₁ is

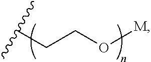

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; and Z may be

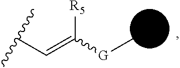

wherein Q is

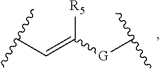

R₅ is as defined herein, preferably R₅ is H; G is S, O or NR₁₀, wherein R₁₀ is as defined herein, preferably R₁₀ is H; and ● is as defined herein; preferably, Z is

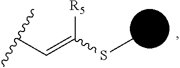

wherein Q is

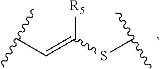

R₅ is as defined herein, preferably R₅ is H, and ● is as defined herein. Preferably, in any one of these embodiments Y is O. Optionally, in any one of these embodiments, a linker may be arranged between ● and Q.

In any one of the compounds of formula (I) or (III), R₁ may be C₁-C₈-alkyl, preferably methyl, ethyl, propyl or butyl; more preferably methyl or ethyl; still more preferably ethyl; and Z may be selected from the group consisting of

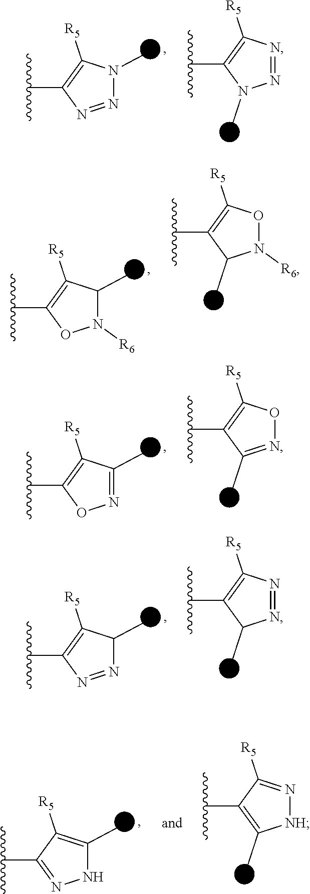

wherein R₅ is as defined herein, preferably R₅ is H; R₆ is as defined herein; and ● is as defined herein. More preferably, Z is

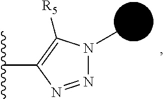

wherein Q is

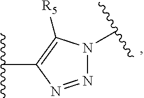

R₅ is as defined herein, preferably R₅ is H; and ● is as defined herein. Preferably, Z is

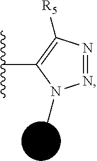

wherein Q is

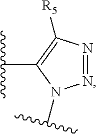

R₅ is as defined herein, preferably R₅ is H; and ● is as defined herein. Z may be

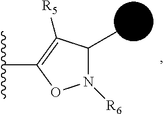

wherein Q is

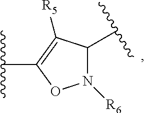

R₅ is as defined herein, preferably R₅ is H; R₆ is as defined herein; and ● is as defined herein. Z may be

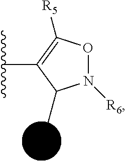

wherein Q is

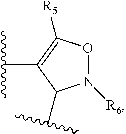

R₅ is as defined herein, preferably R₅ is H; R₆ is as defined herein; and ● is as defined herein. Z may be

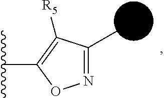

wherein Q is

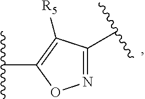

R₅ is as defined herein, preferably R₅ is H; and ● is as defined herein. Z may be

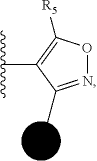

wherein Q is

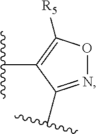

R₅ is as defined herein, preferably R₅ is H; and ● is as defined herein. Z may be

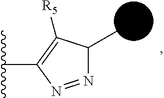

wherein Q is

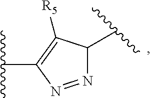

R₅ is as defined herein, preferably R₅ is H; and ● is as defined herein. Z may be

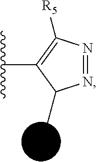

wherein Q is

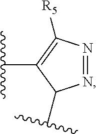

R₅ is as defined herein, preferably R₅ is H; and ● is as defined herein. Z may be

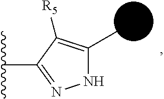

wherein Q is

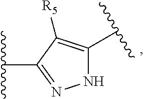

R₅ is as defined herein, preferably R₅ is H; and ● is as defined herein. Z may be

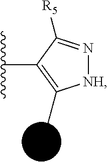

wherein Q is

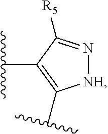

R₅ is as defined herein, preferably R₅ is H; and ● is as defined herein. Preferably, in any one of these embodiments Y is O. Optionally, in any one of these embodiments, R₁ may be C₁-C₈-alkyl substituted with a fluorophore. Optionally, in any one of these embodiments, a linker may be arranged between ● and Q.

In any one of the compounds of formula (I) or (III), R₁ may be C₂-C₈-alkynyl, preferably

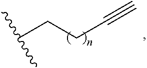

wherein n is 1, 2, 3, 4, 5, 6, 7, 8, 9 or 10, preferably 1, 2 or 3, more preferably 1; and Z may be selected from the group consisting of

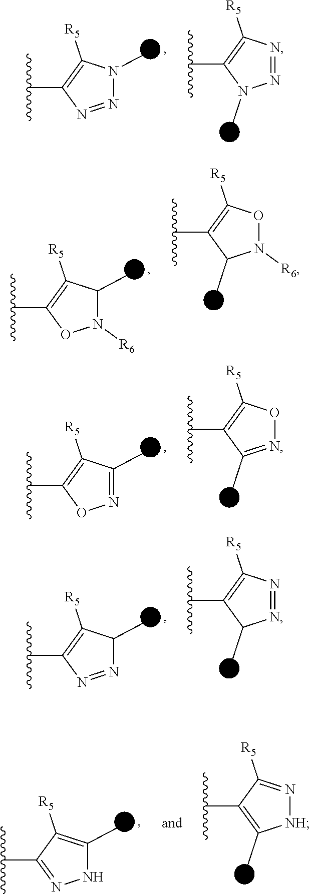

wherein R₅ is as defined herein, preferably R₅ is H; R₆ is as defined herein; and ● is as defined herein. More preferably, Z is

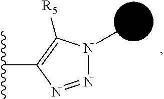

wherein Q is

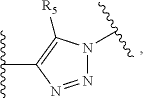

R₅ is as defined herein, preferably R₅ is H; and ● is as defined herein. Preferably, Z is

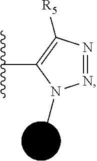

wherein Q is

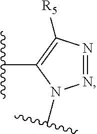

R₅ is as defined herein, preferably R₅ is H; and ● is as defined herein. Z may be

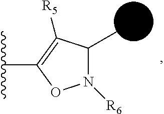

wherein Q is

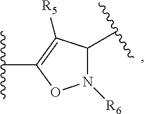

R₅ is as defined herein, preferably R₅ is H; R₆ is as defined herein; and ● is as defined herein. Z may be

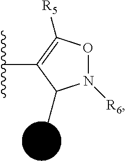

wherein Q is

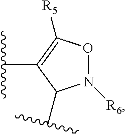

R₅ is as defined herein, preferably R₅ is H; R₆ is as defined herein; and ● is as defined herein. Z may be

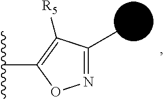

wherein Q is

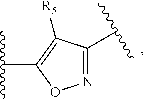

R₅ is as defined herein, preferably R₅ is H; and ● is as defined herein. Z may be

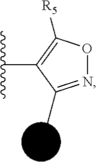

wherein Q is

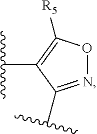

R₅ is as defined herein, preferably R₅ is H; and ● is as defined herein. Z may be

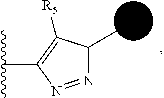

wherein Q is

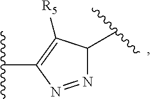

R₅ is as defined herein, preferably R₅ is H; and ● is as defined herein. Z may be

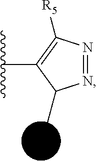

wherein Q is

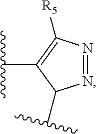

R₅ is as defined herein, preferably R₅ is H; and ● is as defined herein. Z may be

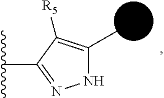

wherein Q is

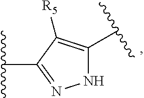

R₅ is as defined herein, preferably R₅ is H; and ● is as defined herein. Z may be

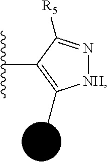

wherein Q is

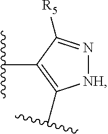

R₅ is as defined herein, preferably R₅ is H; and ● is as defined herein. Preferably, in any one of these embodiments Y is O. Optionally, in any one of these embodiments, a linker may be arranged between ● and Q.

In any one of the compounds of formula (I) or (III), R₁ may be

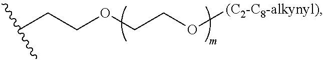

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3; preferably R₁ may be

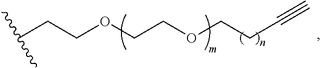

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and Z may be selected from the group consisting of

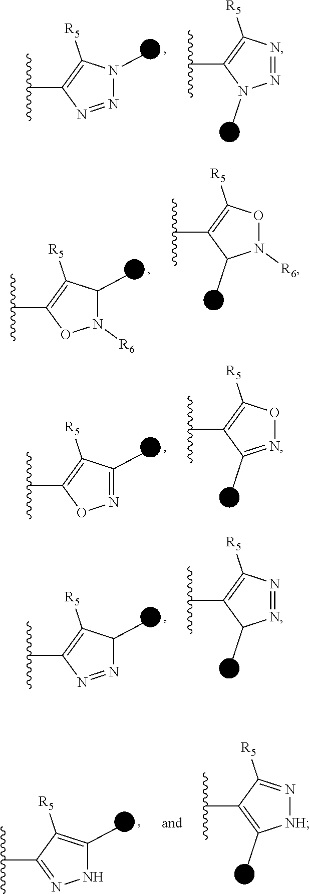

wherein R₅ is as defined herein, preferably R₅ is H; R₆ is as defined herein; and ● is as defined herein. More preferably, Z is

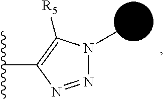

wherein Q is

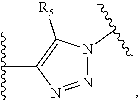

R₅ is as defined herein, preferably R₅ is H; and ● is as defined herein. Preferably, Z is

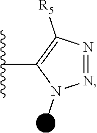

wherein Q is

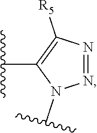

R₅ is as defined herein, preferably R₅ is H; and ● is as defined herein. Z may be

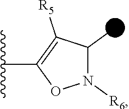

wherein Q is

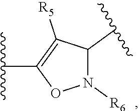

R₅ is as defined herein, preferably R₅ is H; R₆ is as defined herein; and ● is as defined herein. Z may be

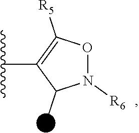

wherein Q is

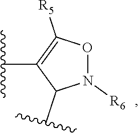

R₅ is as defined herein, preferably R₅ is H; R₆ is as defined herein; and ● is as defined herein. Z may be

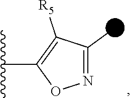

wherein Q is

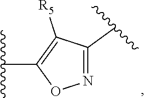

R₅ is as defined herein, preferably R₅ is H; and ● is as defined herein. Z may be

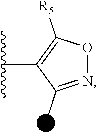

wherein Q is

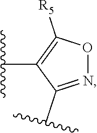

R₅ is as defined herein, preferably R₅ is H; and ● is as defined herein. Z may be

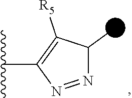

wherein Q is

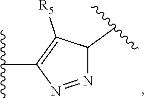

R₅ is as defined herein, preferably R₅ is H; and ● is as defined herein. Z may be

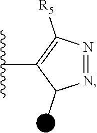

wherein Q is

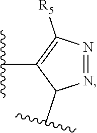

R₅ is as defined herein, preferably R₅ is H; and ● is as defined herein. Z may be

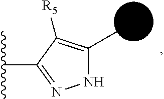

wherein Q is

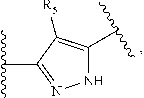

R₅ is as defined herein, preferably R₅ is H; and ● is as defined herein. Z may be

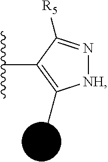

wherein Q is

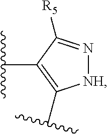

R₅ is as defined herein, preferably R₅ is H; and ● is as defined herein. Preferably, in any one of these embodiments Y is O. Optionally, in any one of these embodiments, a linker may be arranged between ● and Q.

In any one of the compounds of formula (I) or (III), R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be selected from the group consisting of

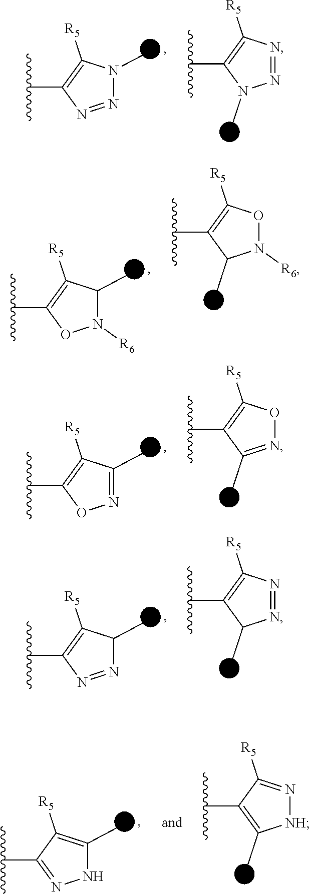

wherein R₅ is as defined herein, preferably R₅ is H; R₆ is as defined herein; and ● is as defined herein. More preferably, Z is

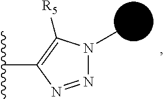

wherein Q is

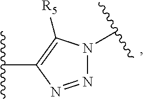

R₅ is as defined herein, preferably R₅ is H; and ● is as defined herein. Preferably, Z is

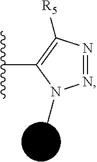

wherein Q is

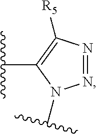

R₅ is as defined herein, preferably R₅ is H; and ● is as defined herein. Z may be

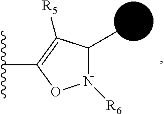

wherein Q is

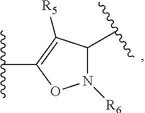

R₅ is as defined herein, preferably R₅ is H; R₆ is as defined herein; and ● is as defined herein. Z may be

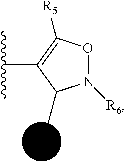

wherein Q is

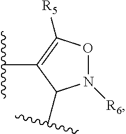

R₅ is as defined herein, preferably R₅ is H; R₆ is as defined herein; and ● is as defined herein. Z may be

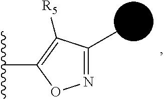

wherein Q is

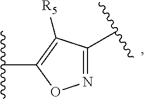

R₅ is as defined herein, preferably R₅ is H; and ● is as defined herein. Z may be

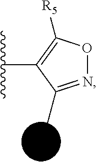

wherein Q is

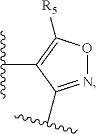

R₅ is as defined herein, preferably R₅ is H; and ● is as defined herein. Z may be

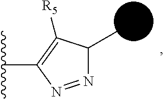

wherein Q is

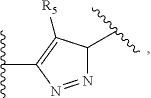

R₅ is as defined herein, preferably R₅ is H; and ● is as defined herein. Z may be

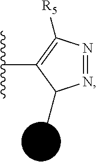

wherein Q is

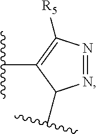

R₅ is as defined herein, preferably R₅ is H; and ● is as defined herein. Z may be

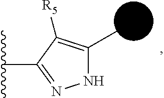

wherein Q is

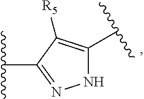

R₅ is as defined herein, preferably R₅ is H; and ● is as defined herein. Z may be

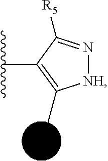

wherein Q is

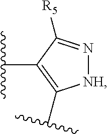

R₅ is as defined herein, preferably R₅ is H; and ● is as defined herein. Optionally, in any one of these embodiments Y is O. Optionally, in any one of these embodiments, a linker may be arranged between ● and Q.

In any one of the compounds of formula (I) or (III), R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z may be selected from the group consisting of

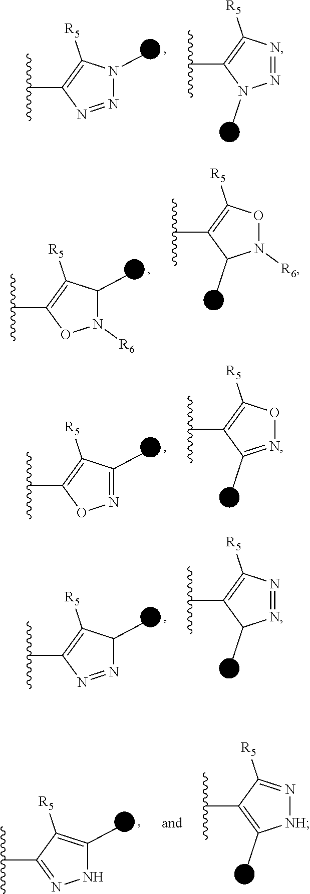

wherein R₅ is as defined herein, preferably R₅ is H; R₆ is as defined herein; and ● is as defined herein. More preferably, Z is

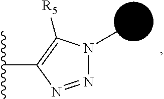

wherein Q is

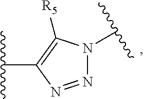

R₅ is as defined herein, preferably R₅ is H; and ● is as defined herein. Preferably, Z is

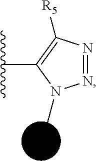

wherein Q is

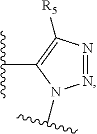

R₅ is as defined herein, preferably R₅ is H; and ● is as defined herein. Z may be

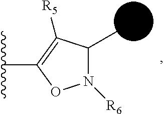

wherein Q is

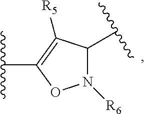

R₅ is as defined herein, preferably R₅ is H; R₆ is as defined herein; and ● is as defined herein. Z may be

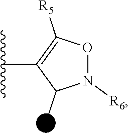

wherein Q is

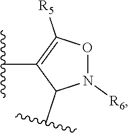

R₅ is as defined herein, preferably R₅ is H; R₆ is as defined herein; and ● is as defined herein. Z may be

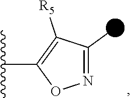

wherein Q is

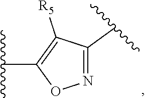

R₅ is as defined herein, preferably R₅ is H; and ● is as defined herein. Z may be

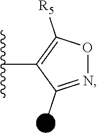

wherein Q is

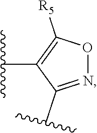

R₅ is as defined herein, preferably R₅ is H; and ● is as defined herein. Z may be

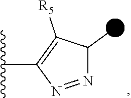

wherein Q is

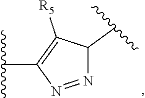

R₅ is as defined herein, preferably R₅ is H; and ● is as defined herein. Z may be

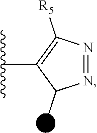

wherein Q is

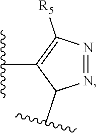

R₅ is as defined herein, preferably R₅ is H; and ● is as defined herein. Z may be

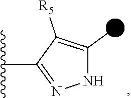

wherein Q is

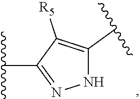

R₅ is as defined herein, preferably R₅ is H; and ● is as defined herein. Z may be

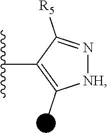

wherein Q is

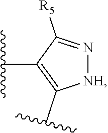

R₅ is as defined herein, preferably R₅ is H; and ● is as defined herein. Optionally, in any one of these embodiments Y is O. Optionally, in any one of these embodiments, a linker may be arranged between ● and Q.

Preferably, in any one of the compounds of formula (I) or (III), R₁ is

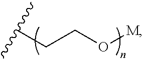

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; and Z may be selected from the group consisting of

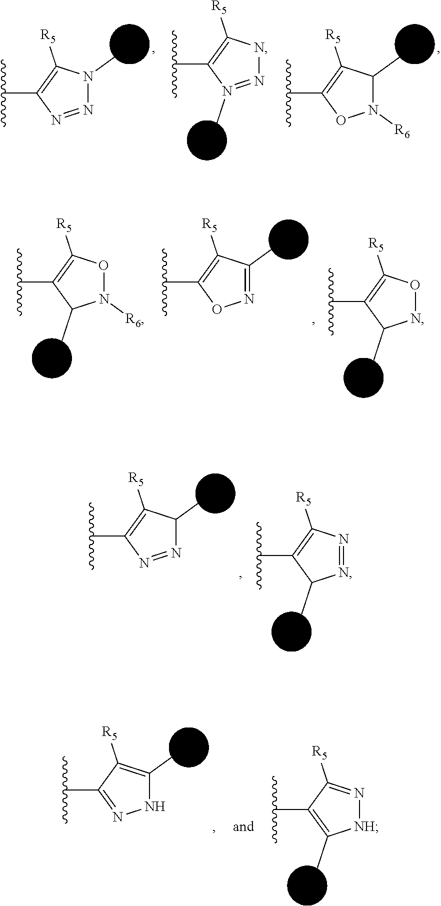

wherein R₅ is as defined herein, preferably R₅ is H; R₆ is as defined herein; and ● is as defined herein. More preferably, Z is

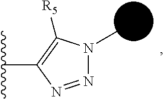

wherein Q is

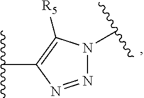

is as defined herein, preferably R₅ is H; and ● is as defined herein. Preferably, Z is

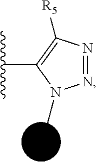

wherein Q is

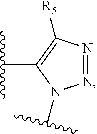

R₅ is as defined herein, preferably R₅ is H; and ● is as defined herein. Z may be

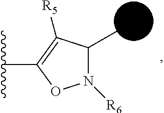

wherein Q is

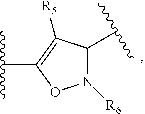

R₅ is as defined herein, preferably R₅ is H; R₆ is as defined herein; and ● is as defined herein. Z may be

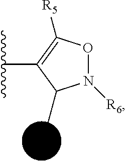

wherein Q is

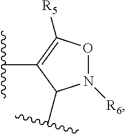

R₅ is as defined herein, preferably R₅ is H; R₆ is as defined herein; and ● is as defined herein. Z may be

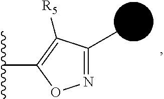

wherein Q is

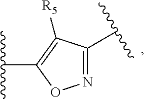

R₅ is as defined herein, preferably R₅ is H; and S is as defined herein. Z may be

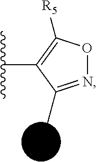

wherein Q is

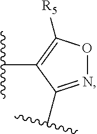

R₅ is as defined herein, preferably R₅ is H; and ● is as defined herein. Z may be

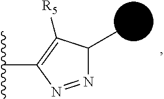

wherein Q is

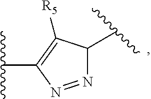

R₅ is as defined herein, preferably R₅ is H; and ● is as defined herein. Z may be

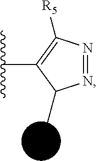

wherein Q is

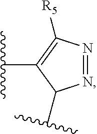

R₅ is as defined herein, preferably R₅ is H; and ● is as defined herein. Z may be

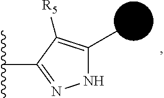

wherein Q is

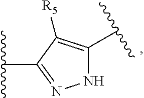

R₅ is as defined herein, preferably R₅ is H; and ● is as defined herein. Z may be

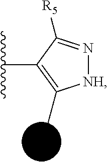

wherein Q is

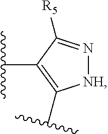

R₅ is as defined herein, preferably R₅ is H; and ● is as defined herein. Optionally, in any one of these embodiments Y is O. Optionally, in any one of these embodiments, a linker may be arranged between ● and Q.

In any one of the compounds of formula (I) or (III), R₁ may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

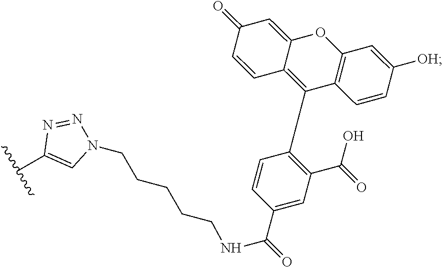 [SMILES: *c1cn(CCCCCNC(=O)c2ccc(-c3c4ccc(=O)cc-4oc4cc(O)ccc34)c(C(=O)O)c2)nn1]

or still more preferably R₁ is

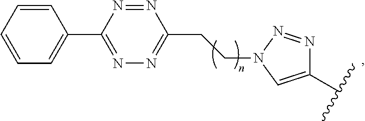

wherein n is 1, 2, 3, 4, 5, 6, 7, 8 or 9, preferably 1, 2 or 3, more preferably 1; or preferably R₁ is

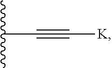

wherein K is as defined herein, preferably K is H; and Z is

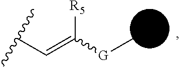

wherein Q is

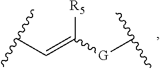

R₅ is as defined herein, preferably R₅ is H; G is S, O or NR₁₀, wherein R₁₀ is as defined herein, preferably R₁₀ is H; and ● is as defined herein. Preferably, Z is

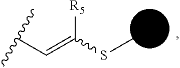

wherein Q is

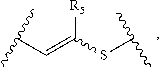

R₅ is as defined herein, preferably R₅ is H, and ● is as defined herein. R₁ may be optionally substituted aryl. Preferably, R₁ is optionally substituted phenyl. More preferably, R₁ is unsubstituted phenyl. R₁ may be optionally substituted heteroaryl. Preferably, R₁ is optionally substituted triazolyl. More preferably, R₁ is triazolyl substituted with optionally substituted C₁-C₈alkyl. R₁ may be a fluorophore. More preferably, R₁ is triazolyl substituted with a fluorophore. Still more preferably, R₁ is

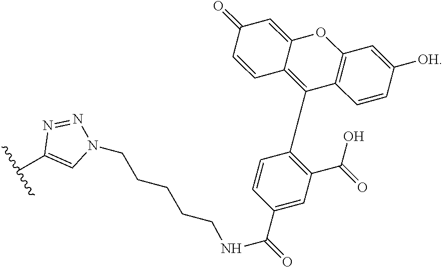 [SMILES: *c1cn(CCCCCNC(=O)c2ccc(-c3c4ccc(=O)cc-4oc4cc(O)ccc34)c(C(=O)O)c2)nn1]

Still more preferably, R₁ is

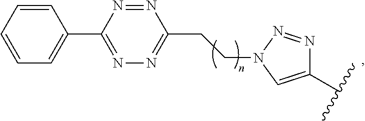

wherein n is 1, 2, 3, 4, 5, 6, 7, 8 or 9, preferably 1, 2 or 3, more preferably 1. Preferably R₁ is

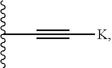

wherein K is H or C₁-C₈alkyl, preferably H or C₁-C₆-alkyl, more preferably H or C₁-C₄-alkyl, still more preferably H or C₁-C₂-alkyl; even more preferably K is H. Preferably, in any one of these embodiments Y is a bond. Optionally, in any one of these embodiments, a linker may be arranged between ● and Q.

In any one of the compounds of formula (I) or (III), ● may represent an amino acid, a peptide, a protein, an antibody, a nucleotide, an oligonucleotide, a saccharide, a polysaccharide, a detectable label, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments, ● represents a protein tag. Preferably, ● represents a fluorophore such as CY₅, fluorescein or EDANS. Preferably, ● represents biotin. Preferably, ● represents a linker. Preferably, S represents a drug. Preferably, ● represents a linker-drug conjugate. Preferably, S represents a linker-fluorophore conjugate. In some embodiments ● represents a polymer. In some embodiments, ● represents a small molecule. In some embodiments, ● represents an optionally substituted C₁-C₈-alkyl, preferably an optionally substituted C₁-C₄-alkyl, more preferably an optionally substituted C₁-C₂alkyl. In some embodiments, ● represents an optionally substituted phenyl. Preferably, ● represents an optionally substituted aromatic 5- or 6-membered heterocyclic system. Optionally, in any one of these embodiments a linker may be arranged between ● and Q.

In any one of the compounds of formula (I) or (III), ● may represent an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments ● represents a polymer. In some embodiments, ● represents an optionally substituted C₁-C₈-alkyl, preferably an optionally substituted C₁-C₄-alkyl, more preferably an optionally substituted C₁-C₂-alkyl. In some embodiments, ● represents an optionally substituted phenyl. Preferably, ● represents an optionally substituted aromatic 5- or 6-membered heterocyclic system. Optionally, in any one of these embodiments a linker may be arranged between ● and Q.

Preferably, in any one of the compounds of formula (I) or (III), ● represents an amino acid, a peptide, a protein, an antibody, a nucleotide, or an oligonucleotide; wherein optionally a linker is arranged between ● and Q. More preferably, ● represents a peptide, a protein, an antibody, or an oligonucleotide; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. Optionally, in any one of these embodiments a linker may be arranged between ● and Q.

Preferably, in any one of the compounds of formula (I) or (III), ● represents a drug, a protein tag, or a fluorophore such as CY₅, fluorescein or EDANS, biotin, a protein, a peptide, an antibody or an oligonucleotide; wherein optionally a linker is arranged between ● and Q. Preferably, ● represents a drug. Preferably, ● represents a protein tag. Preferably, ● represents a linker-drug conjugate. Preferably, ● represents a fluorophore such as CY₅, fluorescein or EDANS. Preferably, ● represents biotin. Preferably, ● represents a protein. Preferably, ● represents a peptide. Preferably, ● represents an antibody. Preferably, ● represents an oligonucleotide. Optionally, in any one of these embodiments a linker may be arranged between ● and Q.

Preferably, in any one of the compounds of formula (I) or (III), ● represents a linker, a drug, or a linker-drug conjugate. Preferably, ● represents a linker. Preferably, ● represents a drug. Preferably, ● represents a linker-drug conjugate. The linker, drug or linker-drug conjugate may be any linker, drug or linker-drug conjugate as described herein. In particular, the linker, drug, or linker-drug conjugate may be any linker, drug or linker drug conjugate as described herein with regard to embodiments where ● represents a linker, a drug or a linker-drug conjugate.

In some embodiments, when ● represents a linker, a drug or a linker-drug conjugate, [symbol] may represent an antibody.

Preferably, in any one of the compounds of formula (I) or (III), ● represents a detectable label. Optionally, in this embodiment, a linker may be arranged between ● and Q.

Preferably, in any one of the compounds of formula (I) or (III), ● represents a linker, a fluorophore, or a linker-fluorophore conjugate. Preferably, ● represents a linker. Preferably, ● represents a fluorophore. Preferably, ● represents a linker-fluorophore conjugate.

Preferably, in any one of the compounds of formula (I) or (III), ● represents a small molecule, a fluorophore, a peptide, a protein, or an antibody; wherein optionally a linker is arranged between ● and Q. Preferably, ● represents a small molecule. Preferably, ● represents a fluorophore. Preferably, ● represents a peptide. Preferably, ● represents a protein. Preferably, ● represents an antibody. Optionally, in any one of these embodiments a linker may be arranged between ● and Q.

In any one of the compounds of formula (III), [symbol] may represent an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system. Preferably, [symbol] represents an amino acid, a peptide, a protein, an antibody, a nucleotide, an oligonucleotide, or a small molecule. More preferably, [symbol] represents a peptide, a protein, an antibody, an oligonucleotide, or a small molecule. Preferably, [symbol] represents an amino acid. Preferably, [symbol] represents a peptide. Preferably, [symbol] represents a protein. Preferably, [symbol] represents an antibody. Preferably, [symbol] represents a nucleotide. Preferably, [symbol] represents an oligonucleotide. In some embodiments [symbol] represents a saccharide. In some embodiments [symbol] represents a polysaccharide. In some embodiments [symbol] represents a polymer. Preferably, [symbol] represents a small molecule. In some embodiments [symbol] represents an optionally substituted C₁-C₈-alkyl, preferably an optionally substituted C₁-C₆alkyl, more preferably an optionally substituted C₁-C₄-alkyl, still more preferably an optionally substituted C₁-C₂alkyl. In some embodiments [symbol] represents an optionally substituted C₃-C₈-alkyl, preferably an optionally substituted C₃-C₆-alkyl, more preferably an optionally substituted C₃-C₄-alkyl. In some embodiments [symbol] represents an optionally substituted C₅-C₈-alkyl, preferably an optionally substituted C₆-C₇-alkyl. In some embodiments [symbol] represents an optionally substituted phenyl. In some embodiments [symbol] represents an optionally substituted aromatic 5- or 6-membered heterocyclic system.

Preferably, in any one of the compounds of formula (III), [symbol] represents an antibody, preferably an IgG antibody, such as e.g. a Cetuximab or a Trastuzumab or a Brentuximab; a protein, preferably a GFP protein or eGFP-protein, an mCherry protein or an albumin; a small molecule; a peptide, preferably a peptide of formula (VIII)

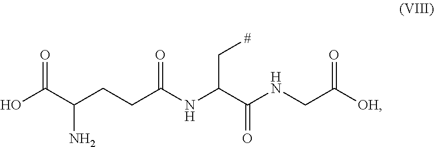

or of formula (IX).

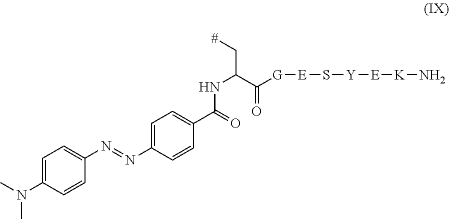

wherein #represents the position of S. Preferably, [symbol] represents an antibody, such as e.g. a Cetuximab or a Trastuzumab or a Brentuximab. Preferably, [symbol] represents a protein, such as e.g. a GFP protein or eGFP-protein. In some embodiments [symbol] represents an mCherry protein. In some embodiments [symbol] represents albumin. Preferably, [symbol] represents a small molecule. Preferably, [symbol] represents a peptide. More preferably, [symbol] represents a peptide of formula (VIII)

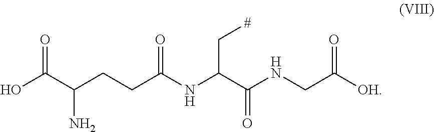

More preferably, [symbol] represents a peptide of formula (IX)

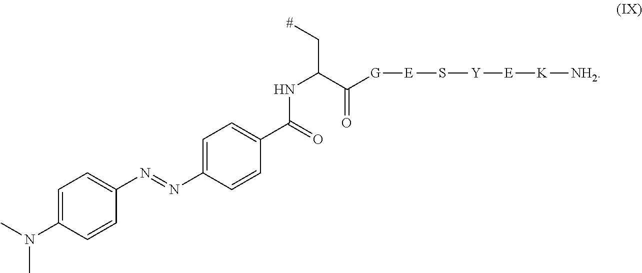

Preferably, in any one of the compounds of formula (III), [symbol] represents an antibody (e.g. a Cetuximab, Trastuzumab, or Brentuximab) and ● represents a protein tag, or a fluorophore such as CY₅, fluorescein or EDANS, biotin, a peptide, a protein, an oligonucleotide, or a small molecule; wherein optionally a linker is arranged between ● and Q. Preferably, [symbol] represents an antibody and ● represents a protein tag. Preferably, [symbol] represents an antibody and ● represents a fluorophore such as CY₅, fluorescein or EDANS. Preferably, [symbol] represents an antibody and ● represents biotin. Preferably, [symbol] represents an antibody and ● represents a peptide. Preferably, represents an antibody and ● represents a protein. Preferably, [symbol] represents an antibody and ● represents an oligonucleotide. Preferably, [symbol] represents an antibody and ● represents a small molecule. Optionally, in any one of these embodiments a linker may be arranged between ● and Q.

Preferably, in any one of the compounds of formula (III), [symbol] represents a protein (e.g. a GFP protein or eGFP protein or mCherry proterin) and ● represents a protein tag, or a fluorophore such as CY₅, fluorescein or EDANS, biotin, a peptide, an antibody, a protein, an oligonucleotide, or a small molecule; wherein optionally a linker is arranged between ● and Q. Preferably, [symbol] represents a protein and ● represents a protein tag. Preferably, [symbol] represents a protein and ● represents a fluorophore such as CY₅, fluorescein or EDANS. Preferably, [symbol] represents a protein and ● represents biotin. Preferably, [symbol] represents a protein and ● represents a peptide. Preferably, [symbol] represents a protein and ● represents an antibody. Preferably, [symbol] represents a protein and ● represents a protein. Preferably, [symbol] represents a protein and ● represents an oligonucleotide. Preferably, [symbol] represents a protein and ● represents a small molecule Optionally, in any one of these embodiments a linker may be arranged between ● and Q.

Preferably, in any one of the compounds of formula (III), [symbol] represents a peptide and ● represents a protein tag, or a fluorophore such as CY₅, fluorescein or EDANS, biotin, a peptide, a protein, an oligonucleotide, or a small molecule; wherein optionally a linker is arranged between ● and Q. Preferably, [symbol] represents a peptide and ● represents a protein tag. Preferably, [symbol] represents a peptide and ● represents a fluorophore such as CY₅ or EDANS. Preferably, [symbol] represents a peptide and ● represents biotin. Preferably, [symbol] represents a peptide and ● represents a peptide. Preferably, [symbol] represents a peptide and ● represents a protein. Preferably, [symbol] represents a peptide and ● represents an oligonucleotide. Preferably, [symbol] represents a peptide and ● represents a small molecule. Optionally, in any one of these embodiments a linker may be arranged between ● and Q.

Preferably, in any one of the compounds of formula (III), [symbol] represents an amino acid and ● represents a protein tag, or a fluorophore such as CY₅, fluorescein or EDANS, biotin, a peptide, a protein, an oligonucleotide, or a small molecule; wherein optionally a linker is arranged between ● and Q. Preferably, [symbol] represents an amino acid and ● represents a protein tag. Preferably, [symbol] represents an amino acid and ● represents a fluorophore such as CY₅, fluorescein or EDANS. Preferably, [symbol] represents an amino acid and ● represents biotin. Preferably, [symbol] represents an amino acid and ● represents a peptide. Preferably, [symbol] represents an amino acid and ● represents a protein. Preferably, [symbol] represents an amino acid and ● represents an oligonucleotide. Preferably, [symbol] represents an amino acid and ● represents a small molecule. Optionally, in any one of these embodiments a linker may be arranged between ● and Q.

Preferably, in any one of the compounds of formula (III), [symbol] represents an antibody (e.g. a Cetuximab, a Trastuzumab, or a Brentuximab) and ● represents a linker, a drug, or a linker-drug conjugate. Preferably, [symbol] represents an antibody and ● represents a linker. Preferably, [symbol] represents an antibody and ● represents a drug. Preferably, [symbol] represents an antibody and ● represents a linker-drug conjugate. The linker, drug or linker-drug conjugate may be any linker, drug or linker-drug conjugate as described herein. In particular, the linker, drug, or linker-drug conjugate may be any linker, drug or linker drug conjugate as described herein with regard to embodiments where ● represents a linker, a drug or a linker-drug conjugate.

Preferably, in any one of the compounds of formula (III), [symbol] represents an antibody (e.g. a Cetuximab, a Trastuzumab, or a Brentuximab) and ● represents a linker, a fluorophore, or a linker-fluorophore conjugate. Preferably, [symbol] represents an antibody and ● represents a linker. Preferably, represents an antibody and ● represents a fluorophore. Preferably, [symbol] represents an antibody and ● represents a linker-fluorophore conjugate.

Preferably, in any one of the compounds of formula (III), [symbol] represents a nucleotide and ● represents a peptide, a protein, a protein tag, an antibody, an oligonucleotide, a fluorophore such as CY₅, fluorescein, or EDANS, biotin, or a small molecule; wherein optionally a linker is arranged between ● and Q. Preferably, [symbol] represents a nucleotide and ● represents a peptide. Preferably represents a nucleotide and ● represents a protein. Preferably, [symbol] represents a nucleotide and ● represents a protein tag. Preferably, [symbol] represents a nucleotide and ● represents an antibody. Preferably, [symbol] represents a nucleotide and ● represents an oligonucleotide. Preferably, [symbol] represents nucleotide and ● represents a fluorophore such as CY₅, fluorescein or EDANS. Preferably, [symbol] represents a nucleotide and ● represents biotin. Preferably, [symbol] represents a nucleotide and ● represents a small molecule. Optionally, in any one of these embodiments a linker may be arranged between ● and Q.

Preferably, in any one of the compounds of formula (III), [symbol] represents a nucleotide and ● represents a linker.

Preferably, in any one of the compounds of formula (III), [symbol] represents an oligonucleotide and ● represents a peptide, a protein, a protein tag, an antibody, an oligonucleotide, a fluorophore such as CY₅, fluorescein or EDANS, biotin, or a small molecule; wherein optionally a linker is arranged between ● and Q. Preferably, [symbol] represents an oligonucleotide and ● represents a peptide. Preferably, [symbol] represents an oligonucleotide and ● represents a protein. Preferably, [symbol] represents an oligonucleotide and ● represents a protein tag. Preferably, [symbol] represents an oligonucleotide and ● represents an antibody. Preferably, [symbol] represents an oligonucleotide and S represents an oligonucleotide. Preferably, [symbol] represents an oligonucleotide and S represents a fluorophore such as CY₅, fluorescein, or EDANS. Preferably, [symbol] represents an oligonucleotide and ● represents biotin. Preferably, represents an oligonucleotide and ● represents a small molecule. Optionally, in any one of these embodiments a linker may be arranged between ● and Q.

Preferably, in any one of the compounds of formula (III), represents an oligonucleotide and ● represents a linker.

In some embodiments of any one of the compounds of formula (I) or formula (III), ● represents an amino acid, a peptide, a nucleotide or an oligonucleotide, wherein the amino acid, peptide, nucleotide or oligonucleotide is bound to a solid support. In some embodiments ● represents an amino acid or a peptide bound to a solid support. In some embodiments ● represents a nucleotide or an oligonucleotide bound to a solid support. Preferably, ● represents a peptide bound to a solid support. Compounds of formula (III) of the present invention are stable under acidic conditions which are typically used for cleavage of a peptide from the solid support, e.g. 90% trifluoroacetic acid (TFA). The solid support may be any solid support known to a person skilled in the art which is suitable for solid phase peptide synthesis, or any solid support which is suitable for solid phase oligonucleotide synthesis. Such solid supports are also known as resins. Illustrative examples for a solid support suitable for solid phase peptide synthesis include organic and inorganic supports such as a Merrifield polystyrene resin (copolymer from styrene and 1-2% divinylbenzene), polyacrylamide resins, TentaGel (a graft polymer where polythyleneglycol is grafted to polystyrene), Wang resin (typically based on crosslinked polystyrene, such as in a Merrifield resin), or porous glass having defined pore size as an example for an inorganic solid support. Illustrative examples for commercially available solid supports for solid phase peptide synthesis are Rink amide resins or NovaSyn®TGR resins supplied by Merck Millipore. Illustrative examples for a solid support suitable for solid phase oligonucleotide synthesis include glass having defined pore size (controlled pore glass, CPG) and polystyrene, such as macroporous polystyrene (MPPS). Optionally, in the foregoing embodiments where the amino acid, peptide, nucleotide or oligonucleotide is bound to a solid support, a linker may be arranged between ● and Q. Accordingly, the ●, Q, linker and solid support may be arranged as follows: Q-Unker-Amino Acid-Solid Support, Q-Unker-Peptide-Solid Support, Q-Unker-Nucleotide-Solid Support, or Q-Linker-Oligonucleotide-Solid Support. The “Unker” can be virtually any linker, and the linker is arranged between ● and Q. The Unker may be any linker known to a person skilled in the art, for example, a peptidic linker or a straight or branched hydrocarbon-based moiety. The linker can also comprise cyclic moieties. A peptidic linker may comprise, for example, 1 to 50, 1 to 40, 1 to 30, 1 to 20, 1 to 10, 1 to 5, 1 to 3, or 2, or 1 amino acid(s). If the linker is a hydrocarbon-based moiety, the main chain of the linker may comprise only carbon atoms but can also contain heteroatoms such as oxygen (O), nitrogen (N) or sulfur (S) atoms, and/or contain carbonyl groups (C═O). The linker may be, for example, a C₁-C₂₀ carbon atom chain or a polyether based chain such as a polyethylene glycol-based chain with —(O—CH₂—CH₂)— repeating units. In typical embodiments of hydrocarbon-based linkers, the linking moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. A person skilled in the art knows to select suitable linkers.

In some embodiments of any one of the compounds of formula (III), [symbol] represents an amino acid, a peptide, a nucleotide or an oligonucleotide, wherein the amino acid, peptide nucletide or oligonucleotide is bound to a solid support. In some embodiments [symbol] represents an amino acid or a peptide bound to a solid support. In some embodiments [symbol] represents a nucleotide or an oligonucleotide bound to a solid support. Preferably, [symbol] represents a peptide bound to a solid support.

The invention also relates to a compound of formula (IIIa)

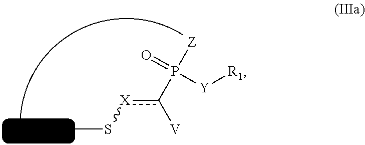

wherein
[symbol] represents a double bond; or
[symbol] represents a bond;
V is absent when [symbol] is a double bond; or
V is H or C₁-C₈-alkyl when [symbol] is a bond;
X represents R₃—C when [symbol] is a double bond; or
X represents

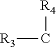

when [symbol] is a bond; and
R₁, R₃, R₄, V, X, Y, Z and [symbol] are as defined herein above and below, in particular as defined with regard to a compound of formula (III). Preferably, the compound (IIIa) is a cyclic peptide, such as for example a cyclic peptide derived from the BCL9 peptide.

The present invention also relates to a compound of formula (IV)

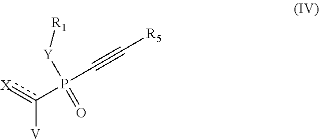

wherein R₁, R₅, [symbol] V, X and Y are as defined herein for any one of the methods and compounds. Accordingly, in any one of the compounds of formula (IV), [symbol] , V, X, Y, R₁ and R₅ may be as defined herein for any one of the methods, compounds and/or conjugates. Any [symbol] , V, X, Y, R₁ and R₅ as defined herein for any one of the methods, compounds and/or conjugates may be combined with each other.

### Compounds of Formula (IV*) and Conjugates of Antibody Molecules

The present invention also relates to a compound of formula (IV*)

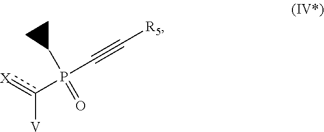

wherein
[symbol] represents a triple bond or a double bond;
V is absent when [symbol] is a triple bond; or
V represents H or C₁-C₈-alkyl when [symbol] is a double bond;
X represents R₃—C when is a triple bond;
X represents

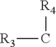

when [symbol] is a double bond;
▴ represents

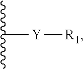

wherein [symbol] indicates the attachment point to the phosphorus; or
▴ represents Z;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
R₅ represents H or C₁-C₈-alkyl; and
Z represents a residue bound to the phosphorus via a carbon atom and comprising a group ●, wherein ● represents an optionally substituted aliphatic or optionally substituted aromatic residue.

In any one of the compounds of formula (IV*), the [symbol] V, X, Y, R₁, R², R₃, R₄, R₅, Z, ▴ and ● may be as defined herein for any one of the methods, compounds and/or conjugates. Any [symbol] , V, X, Y, R₁, R², R₃, R₄, R₅, Z, ▴ and ● as defined herein for any one of the methods, compounds and/or conjugates may be combined with each other.

Preferably, in any one of the compounds of formula (IV*), [symbol] represents a triple bond; V is absent; X represents R₃—C, R₃ represents H or C₁-C₈-alkyl; and R₅ represents H or C₁-C₈-alkyl. Preferably, R₃ represents H or C₁-C₆-alkyl, more preferably H or C₁-C₄-alkyl, still more preferably H or C₁-C₂alkyl. Even more preferably, R₃ is H. Preferably, R₅ represents H or C₁-C₆-alkyl, more preferably R₅ represents H or C₁-C₄-alkyl, still more preferably R₅ represents H or C₁-C₂alkyl. Even more preferably, R₅ is H. Preferably, in any one of the compounds of formula (IV*), when [symbol] is a triple bond and X is R₃—C, R₃ and R₅ are the same; more preferably, when [symbol] is a triple bond and X is R₃—C, R₃ and R₅ are both H.

In some embodiments, in any one of the compounds of formula (IV*), [symbol] may represent a double bond; V may be H or C₁-C₈-alkyl; X may represent

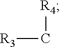

R₃ and R₄ may independently represent H or C₁-C₈-alkyl; and R₅ represents H or C₁-C₈-alkyl. Preferably, R₃ and R₄ independently represent H or C₁-C₆alkyl, more preferably H or C₁-C₄-alkyl, still more preferably H or C₁-C₆alkyl. Preferably, R₃ and R₄ are the same. More preferably, R₃ and R₄ are both H. Preferably, V is H or C₁-C₆-alkyl, more preferably H or C₁-C₄-alkyl, still more preferably H or C₁-C₂alkyl. Even more preferably, V is H. In preferred embodiments, R₃, R₄ and V are each H. Preferably, in any one of the compounds of formula (IV*), when [symbol] is a double bond and X is

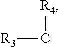

R₃, R₄ and R₅ are the same; even more preferably, R₃, R₄, R₅ and V are the same. More preferably, when [symbol] is a double bond and X is

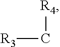

R₃, R₄ and R₅ are each H; more preferably, R₃, R₄, R₅ and V are each H.

The present invention also relates to a conjugate of an antibody molecule comprising at least one moiety of formula (V)

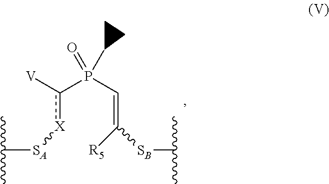

wherein S_{A} and S_{B} are each sulfur atoms of a chain of the antibody molecule;
[symbol] represents a double bond; or
[symbol] represents a bond;
V is absent when [symbol] is a double bond; or
V represents H or C₁-C₈-alkyl when [symbol] is a bond;
X represents R₃—C when [symbol] is a double bond; or
X represents

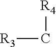

when [symbol] is a bond;
▴ represents

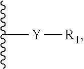

wherein [symbol] indicates the attachment point to the phosphorus; or
▴ represents Z;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl;
R₅ represents H or C₁-C₈-alkyl; and
Z represents a residue bound to the phosphorus via a carbon atom and comprising a group ●, wherein ● represents an optionally substituted aliphatic of optionally substituted aromatic residue.

In any one of the conjugates of an antibody molecule, the [symbol] , V, X, Y, R₁, R², R₃, R₄, R₅, Z, ▴ and ● may be as defined herein for any one of the methods, compounds and/or conjugates. Any [symbol] V, X, Y, R₁, R², R₃, R₄, R₅, Z, ▴ and ● as defined herein for any one of the methods, compounds and/or conjugates may be combined with each other.

In any one of the conjugates of an antibody molecule, the antibody molecule may be selected from the group consisting of an IgA, an IgD, an IgE, an IgG, an IgM, a human antibody, a humanized antibody, a chimeric antibody, a monoclonal antibody, and an isolated antibody. Accordingly, the antibody molecule may be an IgA. The antibody molecule may be an IgD. The antibody molecule may be an IgE. The antibody molecule may be an IgM. The antibody molecule may be a human antibody. The antibody molecule may be a humanized antibody. The antibody molecule may be a chimeric antibody. The antibody molecule may be a monoclonal antibody. The antibody molecule may be an isolated antibody. Preferably, the antibody molecule is an IgG, such as e.g. a Trastuzumab, a Cetuximab or a Brentuximab.

Preferably, in any one of the conjugates of an antibody molecule, [symbol] represents a double bond; V is absent; X represents R₃—C, R₃ represents H or C₁-C₈-alkyl; and R₅ represents H or C₁-C₈-alkyl. Preferably, R₃ represents H or C₁-C₆-alkyl, more preferably H or C₁-C₄-alkyl, still more preferably H or C₁-C₂-alkyl. Even more preferably, R₃ is H. Preferably, R₅ represents H or C₁-C₆alkyl, more preferably R₅ represents H or C₁-C₄-alkyl, still more preferably R₅ represents H or C₁-C₂-alkyl. Even more preferably, R₅ is H. Preferably, in any one of the conjugates of an antibody molecule, when [symbol] is a double bond and X is R₃—C, R₃ and R₅ are the same; more preferably, when [symbol] is a double bond and X is R₃—C, R₃ and R₅ are both H.

In some embodiments, in any one of the conjugates of an antibody molecule, [symbol] may represent a bond; V may be H or C₁-C₈-alkyl; X may represent

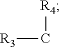

R₃ and R₄ may independently represent H or C₁-C₈-alkyl; and R₅ represents H or C₁-C₈-alkyl. Preferably, R₃ and R₄ independently represent H or C₁-C₆alkyl, more preferably H or C₁-C₄-alkyl, still more preferably H or C₁-C₂alkyl. Preferably, R₃ and R₄ are the same. More preferably, R₃ and R₄ are both H. Preferably, V is H or C₁-C₆-alkyl, more preferably H or C₁-C₄-alkyl, still more preferably H or C₁-C₂alkyl. Even more preferably, V is H. In preferred embodiments, R₃, R₄ and V are the same; more preferably, R₃, R₄ and V are each H. Preferably, in any one of the conjugates of an antibody molecule, when [symbol] is a bond and X is

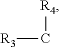

R₃, R₄ and R₅ are the same; even more preferably, R₃, R₄, R₅ and V are the same. More preferably, when [symbol] is a bond and X is

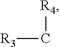

R₃, R₄ and R₅ are each H; even more preferably, R₃, R₄, R₅ and V are each H.

In any one of the compounds of formula (IV*) or the conjugates of an antibody molecule, ▴ may represent

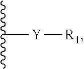

wherein [symbol] indicates the attachment point to the phosphorus; Y represents O, NR², S or a bond; R₁ represents an optionally substituted aliphatic or optionally substituted aromatic residue; and R² represents H or C₁-C₈-alkyl. Preferably, in each instance, R₁ is bound to Y via a carbon atom.

Accordingly, Y may be oxygen (O).

Y may be NR². R² is H or C₁-C₈-alkyl. Preferably, R² is C₁-C₈-alkyl. More preferably, R² is methyl, ethyl, propyl or butyl. Still more preferably, R² is methyl or ethyl.

Y may be S (sulfur).

Y may be a bond. In particular, Y may be a single bond which connects R₁ with the phosphorus.

In any one of the compounds of formula (IV*) or the conjugates of an antibody molecule, R₁ may represent a small molecule; C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; preferably in in each instance Y is O. Accordingly, R₁ may be a small molecule. R₁ may be C₁-C₈-alkyl optionally substituted with (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with at least one of F, Cl, Br, I, —NO₂, —N(C₁-C₈-alkyl)H, —NH₂, —N₃, —N(C₁-C₈-alkyl)₂, ═O, C₁-C₈-cycloalkyl, and/or —S—S—(C₁-C₈-alkyl). R₁ may be C₁-C₈-alkyl optionally substituted with hydroxy-(C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with C₂-C₈-alkenyl. R₁ may be C₁-C₈-alkyl optionally substituted with C₂-C₈-alkynyl. Preferably, in any one of these embodiments Y is O.

In any one of the compounds of formula (IV*) or the conjugates of an antibody molecule, R₁ may represent phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; preferably in any one of these embodiments Y is a bond. Accordingly, R₁ may be phenyl optionally substituted with C₁-C₈-alkyl. R₁ may be phenyl optionally substituted with(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be phenyl optionally substituted with at least one of F, Cl, I, Br, —NO₂, —N(C₁-C₈-alkyl)H, —NH₂, and/or —N(C₁-C₈-alkyl)₂. R₁ may be phenyl optionally substituted with hydroxy-(C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably, in any one of these embodiments Y is a bond.

In any one of the compounds of formula (IV*) or the conjugates of an antibody molecule, R₁ may represent a 5- or 6-membered heteroaromatic system such as optionally substituted triazolyl or optionally substituted pyridyl. Preferably, in any one of these embodiments Y is a bond.

In any one of the compounds of formula (IV*) or the conjugates of an antibody molecule, R₁ may represent a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; or C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₁-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore. Accordingly, R₁ may represent a small molecule, and preferably Y may be O. R₁ may represent C₁-C₈-alkyl, and preferably Y may be O. R₁ may represent C₁-C₈-alkyl substituted with —S—S—(C₁-C₈-alkyl), and preferably Y may be O. R₁ may represent C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₁-C₈-alkyl substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and preferably Y may be O. R₁ may represent C₂-C₈-alkenyl, and preferably Y may be O. R₁ may represent C₁-C₈-alkyl substituted with optionally substituted phenyl, and peferably Y may be O. R₁ may represent C₂-C₈-alkynyl, and preferably Y may be O. R₁ may represent phenyl, and preferably Y may be a bond. R₁ may represent phenyl substituted with —NO₂, and preferably Y may be a bond. R₁ may represent triazolyl substituted with optionally substituted C₁-C₈-alkyl, and preferably Y may be a bond. R₁ may represent triazolyl substituted with a fluorophore, and preferably Y may be a bond.

Preferably, in any one of the compounds of formula (IV*) or the conjugates of an antibody molecule, R₁ may represent C₁-C₈-alkyl. Preferably, R₁ represents methyl, ethyl, propyl or butyl. More preferably, R₁ represents methyl or ethyl. Still more preferably, R₁ represents ethyl. Preferably, in any one of these embodiments R₁ is O.

In any one of the compounds of formula (IV*) or the conjugates of an antibody molecule, R₁ may be selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O. Accordingly, R₁ may be a small molecule. R₁ may be a fluorophore. R₁ may be optionally substituted C₁-C₈-alkyl, preferably methyl, ethyl, propyl or butyl, more preferably methyl or ethyl, still more preferably ethyl. R₁ may be optionally substituted C₂-C₈-alkenyl. R₁ may be optionally substituted optionally substituted C₂-C₈-alkinyl. Preferably, in any one of these embodiments Y is O.

Preferably, in any one of the compounds of formula (IV*) or the conjugates of an antibody molecule, R₁ is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

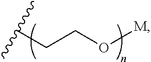

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

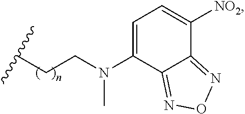

wherein n is 1, 2, 3, 4, 5, 6, 7, 8, 9 or 10, more preferably 4, 5 or 6, still more preferably 5, or more preferably R₁ is

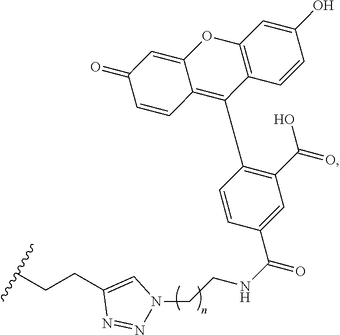

wherein n is 1, 2, 3, 4, 5, 6, 7, 8, 9 or 10, preferably 3, 4 or 5, still more preferably 4; C₂-C₈-alkynyl, preferably

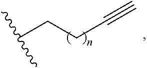

wherein n is 1, 2, 3, 4, or 5, preferably 1, 2 or 3, more preferably 1; or preferably R₁ is

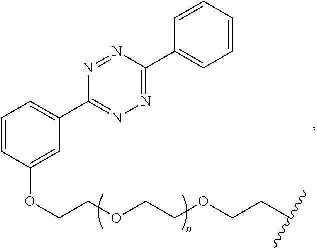

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

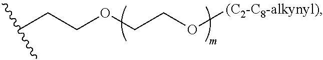

more preferably

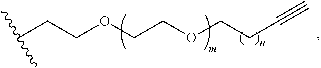

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O. Accordingly, R₁ may be ethyl. R₁ may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. R₁ may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably R₁ is

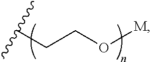

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4. R₁ may be a fluorophore. R₁ may be C₁-C₈-alkyl optionally substituted with a fluorophore. Preferably, R₁ is

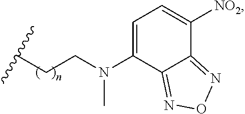

wherein n is 1, 2, 3, 4, 5, 6, 7, 8, 9 or 10, more preferably 4, 5 or 6, still more preferably 5. Preferably, R₁ is

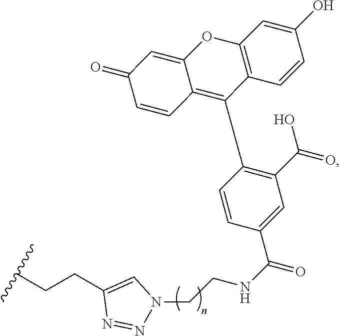

wherein n is 1, 2, 3, 4, 5, 6, 7, 8, 9 or 10, preferably 3, 4 or 5, still more preferably 4. R₁ may be C₂-C₈-alkynyl. Preferably, R₁ is

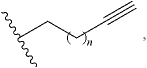

wherein n is 1, 2, 3, 4, or 5, preferably 1, 2 or 3, more preferably 1. Preferably, R₁ is

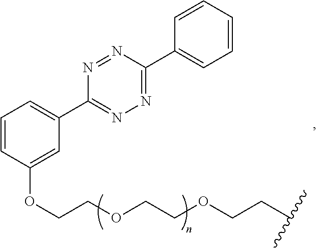

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2. Preferably, R₁ is

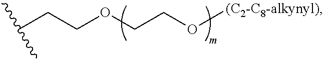

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3. More preferably, R₁ is

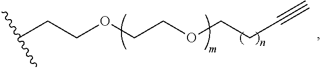

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1. Preferably, in any one of these embodiments Y is O.

Preferably, in any one of the compounds of formula (IV*) or the conjugates of an antibody molecule, R₁ may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

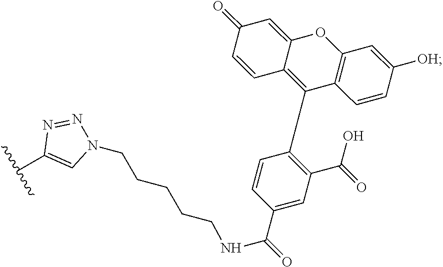 [SMILES: *c1cn(CCCCCNC(=O)c2ccc(-c3c4ccc(=O)cc-4oc4cc(O)ccc34)c(C(=O)O)c2)nn1]

or still more preferably R₁ is

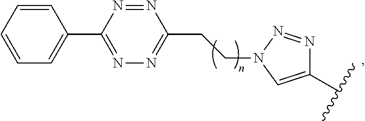

wherein n is 1, 2, 3, 4, 5, 6, 7, 8 or 9, preferably 1, 2 or 3, more preferably 1; or preferably R₁ is

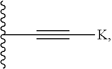

wherein K is H or C₁-C₈-alkyl, preferably K is H; preferably wherein in each instance Y is a bond. Accordingly, R₁ may be optionally substituted aryl. Preferably, R₁ is optionally substituted phenyl. More preferably, R₁ is unsubstituted phenyl. R₁ may be optionally substituted heteroaryl. Preferably, R₁ is optionally substituted triazolyl. More preferably, R₁ is triazolyl substituted with optionally substituted C₁-C₈-alkyl. R₁ may be a fluorophore. More preferably, R₁ is triazolyl substituted with a fluorophore. Still more preferably, R₁ is

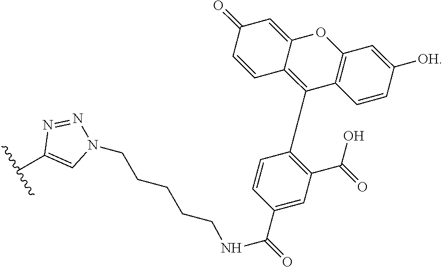 [SMILES: *c1cn(CCCCCNC(=O)c2ccc(-c3c4ccc(=O)cc-4oc4cc(O)ccc34)c(C(=O)O)c2)nn1]

Still more preferably, R₁ is

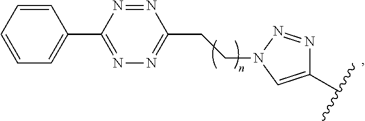

wherein n is 1, 2, 3, 4, 5, 6, 7, 8 or 9, preferably 1, 2 or 3, more preferably 1. Preferably R₁ is

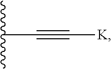

wherein K is H or C₁-C₈-alkyl, preferably H or C₁-C₆-alkyl, more preferably H or C₁-C₄-alkyl, sill more preferably H or C₁-C₂-alkyl; even more preferably K is H. Preferably, in any one of these embodiments Y is a bond.

In any one of the compounds of formula (IV*) or the conjugates of an antibody molecule, R₁ may represent an amino acid, a peptide, a protein, an antibody, a nucleotide, an oligonucleotide, a saccharide, a polysaccharide, a detectable label, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between R₁ and Y. Preferably, R₁ represents an amino acid. Preferably, R₁ represents a peptide. Preferably, R₁ represents a protein. Preferably, R₁ represents an antibody. Preferably, R₁ represents a nucleotide. Preferably, R₁ represents an oligonucleotide. In some embodiments, R₁ represents a saccharide. In some embodiments, R₁ represents a polysaccharide. In some embodiments, R₁ represents a radioactive or non-radioactive nuclide. In some embodiments, R₁ represents a reporter enzyme. In some embodiments, R₁ represents a protein tag. Preferably, R₁ represents a fluorophore such as CY₅, fluorescein or EDANS. Preferably, R₁ represents biotin. Preferably, R₁ represents a linker. Preferably, R₁ represents a drug. Preferably, R₁ represents a linker-drug conjugate. Preferably, R₁ represents a linker-fluorophore conjugate. In some embodiments R₁ represents a polymer. In some embodiments, R₁ represents a small molecule. In some embodiments, R₁ represents an optionally substituted C₁-C₈-alkyl, preferably an optionally substituted C₁-C₄-alkyl, more preferably an optionally substituted C₁-C₂alkyl. In some embodiments, R₁ represents an optionally substituted phenyl. Preferably, R₁ represents an optionally substituted aromatic 5- or 6-membered heterocyclic system. Optionally, in any one of these embodiments a linker may be arranged between R₁ and Y.

In any one of the compounds of formula (IV*) or the conjugates of an antibody molecule, R₁ may represent an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between R₁ and Y. Preferably, R₁ represents an amino acid. Preferably, R₁ represents a peptide. Preferably, R₁ represents a protein. Preferably, R₁ represents an antibody. Preferably, R₁ represents a nucleotide. Preferably, R₁ represents an oligonucleotide. In some embodiments, R₁ represents a saccharide. In some embodiments, R₁ represents a polysaccharide. In some embodiments, R₁ represents a radioactive or non-radioactive nuclide. In some embodiments, R₁ represents a reporter enzyme. In some embodiments R₁ represents a polymer. In some embodiments, R₁ represents an optionally substituted C₁-C₈-alkyl, preferably an optionally substituted C₁-C₄-alkyl, more preferably an optionally substituted C₁-C₂alkyl. In some embodiments, R₁ represents an optionally substituted phenyl. Preferably, R₁ represents an optionally substituted aromatic 5- or 6-membered heterocyclic system. Optionally, in any one of these embodiments a linker may be arranged between R₁ and Y.

Preferably, in any one of the compounds of formula (IV*) or the conjugates of an antibody molecule, R₁ represents an amino acid, a peptide, a protein, an antibody, a nucleotide, or an oligonucleotide; wherein optionally a linker is arranged between R₁ and Y. More preferably, R₁ represents a peptide, a protein, an antibody, or an oligonucleotide; wherein optionally a linker is arranged between R₁ and Y. Preferably, R₁ represents an amino acid. Preferably, R₁ represents a peptide. Preferably, R₁ represents a protein. Preferably, R₁ represents an antibody. Preferably, R₁ represents a nucleotide. Preferably, R₁ represents an oligonucleotide. Optionally, in any one of these embodiments a linker may be arranged between R₁ and Y.

Preferably, in any one of the compounds of formula (IV*) or the conjugates of an antibody molecule, R₁ represents a drug, a protein tag, or a fluorophore such as CY₅, fluorescein or EDANS, biotin, a protein, a peptide, an antibody or an oligonucleotide; wherein optionally a linker is arranged between R₁ and Y. Preferably, R₁ represents a drug. Preferably, R₁ represents a protein tag. Preferably, R₁ represents a linker-drug conjugate. Preferably, R₁ represents a fluorophore such as CY₅, fluorescein or EDANS. Preferably, R₁ represents biotin. Preferably, R₁ represents a protein. Preferably, R₁ represents a peptide. Preferably, R₁ represents an antibody. Preferably, R₁ represents an oligonucleotide. Optionally, in any one of these embodiments a linker may be arranged between R₁ and Y.

Preferably, in any one of the compounds of formula (IV*) or the conjugates of an antibody molecule, R₁ represents a linker, a drug, or a linker-drug conjugate. Preferably, R₁ represents a linker. Preferably, R₁ represents a drug. Preferably, R₁ represents a linker-drug conjugate.

Preferably, in any one of the compounds of formula (IV*) or the conjugates of an antibody molecule, R₁ represents a detectable label. Optionally, in this embodiment, a linker may be arranged between R₁ and Y.

Preferably, in any one of the compounds of formula (IV*) or the conjugates of an antibody molecule, R₁ represents a linker, a fluorophore, or a linker-fluorophore conjugate. Preferably, R₁ represents a linker. Preferably, R₁ represents a fluorophore. Preferably, R₁ represents a linker-fluorophore conjugate.

Preferably, in any one of the compounds of formula (IV*) or the conjugates of an antibody molecule, R₁ represents a small molecule, a fluorophore, a peptide, a protein, or an antibody; wherein optionally a linker is arranged between R₁ and Y. Preferably, R₁ represents a small molecule. Preferably, R₁ represents a fluorophore. Preferably, R₁ represents a peptide. Preferably, R₁ represents a protein. Preferably, R₁ represents an antibody. Optionally, in any one of these embodiments a linker may be arranged between R₁ and Y.

In any one of the compounds of formula (IV*) or the conjugates of an antibody molecule, ▴ may represent Z; and Z represents a residue bound to the phosphorus via a carbon atom and comprising a group ●, wherein ● represents an optionally substituted aliphatic or optionally substituted aromatic residue.

Preferably, in any one of the compounds of formula (IV*) or the conjugates of an antibody molecule, Z is

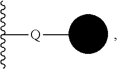

wherein [symbol] indicates the attachment point to the phosphorus and ● is as defined herein; and Q is a moiety comprising at least three main-chain carbon atoms and a carbon-carbon double bond, wherein at least one of the main chain atoms is a heteroatom selected from the group consisting of S, O or N, preferably S. Optionally, in each instance, a linker can be arranged between ● and Q. More preferably, Z is

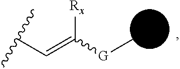

wherein Q is

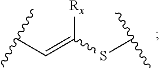

Rₓ is H or C₁-C₈-alkyl; G is S, O or NR₁₀, wherein R₁₀ is H or C₁-C₈-alkyl; and ● is as defined herein; optionally, a linker can be arranged between ● and Q. Preferably, Rₓ is H or C₁-C₆-alkyl, more preferably Rₓ is H or C₁-C₄-alkyl, still more preferably Rₓ is H or C₁-C₂-alkyl. Even more preferably, Rₓ is H. Preferably, in any one of the compounds of formula (IV*) or the conjugates of an antibody molecule, when X is R₃—C, R₃ and Rₓ are the same; more preferably, R₃, Rₓ and R₅ are the same. More preferably, when X is R₃—C, R₃ and Rₓ are both H; even more preferably, R₃, Rₓ and R₅ are each H. Preferably, in any one of the compounds of formula (IV*) or the conjugates of an antibody molecule, when X is

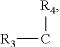

R₃—C, R₃, R₄ and Rₓ are the same; even more preferably, R₃, R₄, Rₓ and R₅ are the same; still more preferably, R₃, R₄, Rₓ, R₅ and V are the same. More preferably, when X is

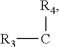

R₃—C, R₃, R₄ and Rₓ are each H; even more preferably, R₃, R₄, Rₓ and R₅ are each H; still more preferably, R₃, R₄, Rₓ, R₅ and V are each H. R₁₀, when present, may be H or C₁-C₆-alkyl, preferably H or C₁-C₄-alkyl, more preferably H or C₁-C₂alkyl. Still more preferably, R₁₀ is H. G may be NR₁₀. G may be O. Preferably, G is S. Accordingly, still more preferably, Z is

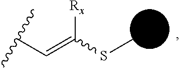

wherein Q is

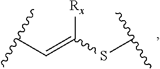

and Rₓ and ● are as defined herein; optionally, a linker can be arranged between ● and Q.

Preferably, in any one of the compounds of formula (IV*) or the conjugates of an antibody molecule, Z is

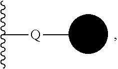

wherein [symbol] indicates the attachment point to the phosphorus and ● is as defined herein; and Q is a five- or six-membered heterocyclic moiety comprising 1, 2 or 3 heteroatoms independently selected from the group consisting of N, O or S. Optionally, in each instance, a linker is arranged between ● and Q. More preferably, Z is selected from the group consisting of

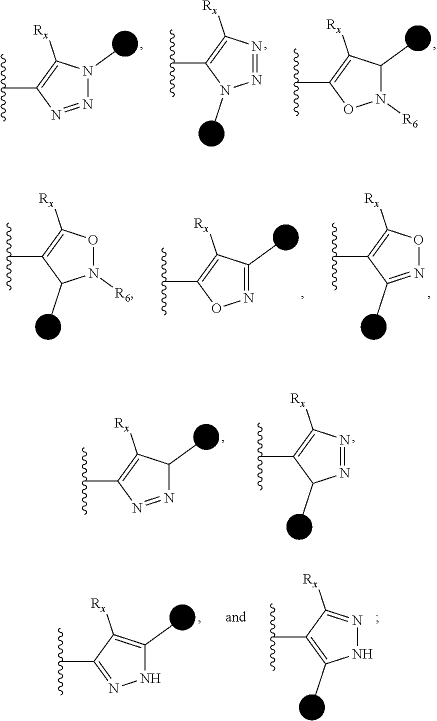

wherein Rₓ is H or C₁-C₈-alkyl; R₆ is C₁-C₈-alkyl, and ● is as defined herein. Accordingly, Z may be

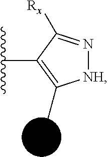

wherein Q is

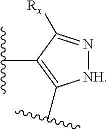

### Z may be

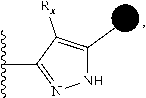

wherein Q is

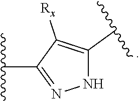

### Z may be

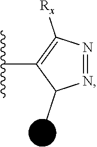

wherein Q is

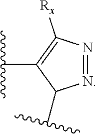

### Z may be

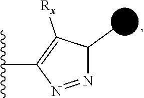

wherein Q is

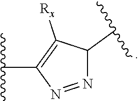

### Z may be

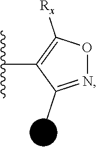

wherein Q is

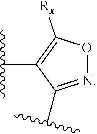

### Z may be

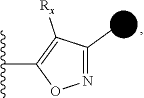

wherein Q is

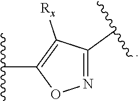

### Z may be

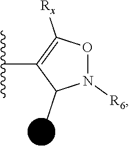

wherein Q is

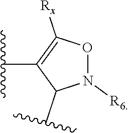

### Z may be

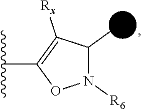

wherein Q is

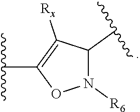

### Preferably, Z is

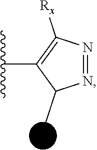

wherein Q is

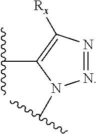

More preferably, Z is

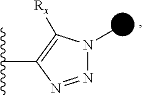

wherein Q is

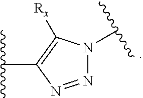

Optionally, in any one of these embodiments a linker can be arranged between ● and Q. Preferably, Rₓ is H or C₁-C₆-alkyl, more preferably H or C₁-C₄-alkyl, still more preferably H or C₁-C₂alkyl. Even more preferably, Rₓ is H. Preferably, in any one of the compounds of formula (IV*) or the conjugates of an antibody molecule, when X is R₃—C, R₃ and Rₓ are the same; more preferably, R₃, Rₓ and R₅ are the same. More preferably, when X is R₃—C, R₃ and Rₓ are both H; even more preferably, R₃, Rₓ and R₅ are each H. Preferably, in any one of the compounds of formula (IV*) or the conjugates of an antibody molecule, when X is

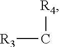

R₃, R₄ and Rₓ are the same; more preferably, R₃, R₄, Rₓ and R₅ are the same; even more preferably, R₃, R₄, Rₓ, R₅ and V are the same. More preferably, when X is

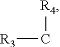

R₃, R₄ and Rₓ are each H; even more preferably, R₃, R₄, Rₓ and R₅ are each H; still more preferably, R₃, R₄, Rₓ, R₅ and V are each H. R₆, when present, may be C₁-C₈-alkyl, preferably C₁-C₆-alkyl, more preferably C₁-C₄-alkyl, still more preferably C₁-C₂alkyl.

Preferably, in any one of the compounds of formula (IV*) or the conjugates of an antibody molecule, Z is

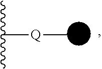

wherein [symbol] indicates the attachment point to the phosphorus and ● is as defined herein; and
Q is a moiety comprising a carbon-carbon triple bond bound to the phosphorus in the compound of formula (IV*) or the moiety of formula (V), and an optionally substituted phenyl group bound to the carbon-carbon triple bond, or
Q is a moiety comprising a carbon-carbon triple bond bound to the phosphorus in formula (IV*) or the moiety of formula (V), and an optionally substituted carbon-carbon double bond bound to the carbon-carbon triple bond. Optionally, in each instance a linker is arranged between ● and Q. More preferably, Z is

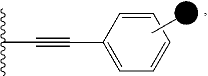

wherein Q is

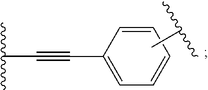

optionally, a linker can be arranged between ● and Q. More preferably, Z is

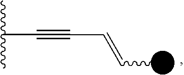

wherein Q is

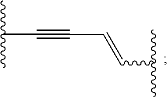

optionally, a linker can be arranged between ● and Q.

In any one of the compounds of formula (IV*) or the conjugates of an antibody molecule, ● may represent an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments, ● represents a protein tag. Preferably, ● represents a fluorophore such as CY₅, fluorescein or EDANS. Preferably, ● represents biotin. Preferably, ● represents a linker. Preferably, ● represents a drug. Preferably, ● represents a linker-drug conjugate. Preferably, ● represents a linker-fluorophore conjugate. In some embodiments ● represents a polymer. In some embodiments, ● represents a small molecule. In some embodiments, ● represents an optionally substituted C₁-C₈-alkyl, preferably an optionally substituted C₁-C₄-alkyl, more preferably an optionally substituted C₁-C₂-alkyl. In some embodiments, ● represents an optionally substituted phenyl. Preferably, ● represents an optionally substituted aromatic 5- or 6-membered heterocyclic system. Optionally, in any one of these embodiments a linker may be arranged between ● and Q.

In any one of the compounds of formula (IV*) or the conjugates of an antibody molecule, ● may represent an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. In some embodiments, ● represents a saccharide. In some embodiments, ● represents a polysaccharide. In some embodiments, ● represents a radioactive or non-radioactive nuclide. In some embodiments, ● represents a reporter enzyme. In some embodiments ● represents a polymer. In some embodiments, ● represents an optionally substituted C₁-C₈-alkyl, preferably an optionally substituted C₁-C₄-alkyl, more preferably an optionally substituted C₁-C₂-alkyl. In some embodiments, ● represents an optionally substituted phenyl. Preferably, ● represents an optionally substituted aromatic 5- or 6-membered heterocyclic system. Optionally, in any one of these embodiments a linker may be arranged between ● and Q.

Preferably, in any one of the compounds of formula (IV*) or the conjugates of an antibody molecule, ● represents an amino acid, a peptide, a protein, an antibody, a nucleotide, or an oligonucleotide; wherein optionally a linker is arranged between ● and Q. More preferably, ● represents a peptide, a protein, an antibody, or an oligonucleotide; wherein optionally a linker is arranged between ● and Q. Preferably, ● represents an amino acid. Preferably, ● represents a peptide. Preferably, ● represents a protein. Preferably, ● represents an antibody. Preferably, ● represents a nucleotide. Preferably, ● represents an oligonucleotide. Optionally, in any one of these embodiments a linker may be arranged between ● and Q.

Preferably, in any one of the compounds of formula (IV*) or the conjugates of an antibody molecule, ● represents a drug, a protein tag, or a fluorophore such as CY₅, fluorescein or EDANS, biotin, a protein, a peptide, an antibody or an oligonucleotide; wherein optionally alinker is arranged between ● and Q. Preferably, ● represents a drug. Preferably, ● represents a protein tag. Preferably, ● represents a linker-drug conjugate. Preferably, ● represents a fluorophore such as CY₅, fluorescein or EDANS. Preferably, ● represents biotin. Preferably, ● represents a protein. Preferably, ● represents a peptide. Preferably, ● represents an antibody. Preferably, ● represents an oligonucleotide. Optionally, in any one of these embodiments a linker may be arranged between ● and Q.

Preferably, in any one of the compounds of formula (IV*) or the conjugates of an antibody molecule, ● represents a linker, a drug, or a linker-drug conjugate. Preferably, ● represents a linker. Preferably, ● represents a drug. Preferably, ● represents a linker-drug conjugate.

Preferably, in any one of the compound of formula (IV*) or the conjugates of an antibody molecule, ● represents a detectable label. Optionally, in this embodiment, a linker may be arranged between ● and Q.

Preferably, in any one of the compounds of formula (IV*) or the conjugates of an antibody molecule, ● represents a linker, a fluorophore, or a linker-fluorophore conjugate. Preferably, ● represents a linker. Preferably, ● represents a fluorophore. Preferably, S represents a linker-fluorophore conjugate.

Preferably, in any one of the compounds of formula (IV*) or the conjugates of an antibody molecule, ● represents a small molecule, a fluorophore, a peptide, a protein, or an antibody; wherein optionally a linker is arranged between ● and Q. Preferably, ● represents a small molecule. Preferably, ● represents a fluorophore. Preferably, ● represents a peptide. Preferably, ● represents a protein. Preferably, ● represents an antibody. Optionally, in any one of these embodiments a linker may be arranged between ● and Q.

In any one of the compounds of formula (IV*) or the conjugates of an antibody molecule, ● may represent a small molecule; C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; wherein optionally a linker is arranged between ● and Q. Accordingly, ● may be a small molecule. ● may be C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be C₁-C₈-alkyl optionally substituted with at least one of F, Cl, Br, I, —NO₂, —N(C₁-C₈-alkyl)H, —NH₂, —N₃, —N(C₁-C₈-alkyl)₂, ═O, C₃-C₈-cycloalkyl, and/or —S—S—(C₁-C₈-alkyl). ● may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be C₁-C₈-alkyl optionally substituted with C₂-C₈-alkenyl. ● may be C₁-C₈-alkyl optionally substituted with C₂-C₈-alkynyl. Optionally, in any one of these embodiments a linker may be arranged between ● and Q.

In any one of the compounds of formula (IV*) or the conjugates of an antibody molecule, 4 may represent phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; wherein optionally a linker is arranged between ● and Q. Accordingly, ● may be phenyl optionally substituted with C₁-C₈-alkyl. ● may be phenyl optionally substituted with(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be phenyl optionally substituted with at least one of F, Cl, I, Br, —NO₂, —N(C₁-C₈-alkyl)H, —NH₂, and/or —N(C₁-C₈-alkyl)₂. ● may be phenyl optionally substituted with hydroxy-(C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Optionally, in any one of these embodiments a linker may be arranged between ● and Q.

In any one of the compounds of formula (IV*) or the conjugates of an antibody molecule, R₁ may represent a 5- or 6-membered heteroaromatic system such as optionally substituted triazolyl or optionally substituted pyridyl. Optionally, in any one of these embodiments a linker may be arranged between ● and Q.

In any one of the compounds of formula (IV*) or the conjugates of an antibody molecule, ● may represent a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; or C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore; wherein optionally a linker is arranged between ● and Q. Accordingly, ● may represent a small molecule. ● may represent C₁-C₈-alkyl. ● may represent C₁-C₈-alkyl substituted with —S—S—(C₁-C₈-alkyl). ● may represent C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may represent C₁-C₈-alkyl substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may represent C₁-C₈-alkenyl. ● may represent C₁-C₈-alkyl substituted with optionally substituted phenyl. ● may represent phenyl. ● may represent phenyl substituted with —NO₂. ● may represent triazolyl substituted with optionally substituted C₁-C₈-alkyl. ● may represent triazolyl substituted with a fluorophore. Optionally, in any one of these embodiments a linker may be arranged between ● and Q.

Preferably, in any one of the compounds of formula (IV*) or the conjugates of an antibody molecule, ● may represent C₁-C₈-alkyl; wherein optionally a linker is arranged between ● and Q. Preferably, ● represents methyl, ethyl, propyl or butyl. More preferably, ● represents methyl or ethyl. Still more preferably, ● represents ethyl. Preferably, in any one of these embodiments R₁ is O. Optionally, in any one of these embodiments a linker may be arranged between ● and Q.

In any one of the compounds of formula (IV*) or the conjugates of an antibody molecule, ● may be selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; wherein optionally a linker is arranged between ● and Q. Accordingly, ● may be a small molecule. ● may be a fluorophore. ● may be optionally substituted C₁-C₈-alkyl, preferably methyl, ethyl, propyl or butyl, more preferably methyl or ethyl, still more preferably ethyl. ● may be optionally substituted C₂-C₈-alkenyl. ● may be optionally substituted optionally substituted C₂-C₈-alkinyl. Optionally, in any one of these embodiments a linker may be arranged between ● and Q.

Preferably, in any one of the compounds of formula (IV*) or the conjugates of an antibody molecule, ● is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably ● is

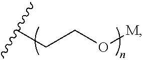

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably ● is

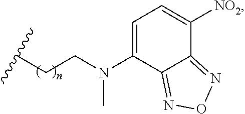

wherein n is 1, 2, 3, 4, 5, 6, 7, 8, 9 or 10, more preferably 4, 5 or 6, still more preferably 5, or more preferably ● is

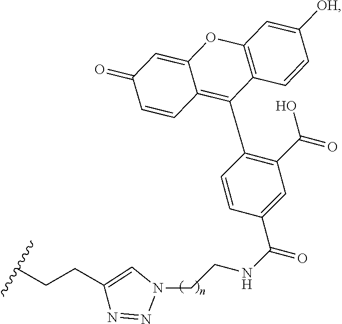

wherein n is 1, 2, 3, 4, 5, 6, 7, 8, 9 or 10, preferably 3, 4 or 5, still more preferably 4; C₂-C₈-alkynyl, preferably

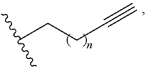

wherein n is 1, 2, 3, 4, or 5, preferably 1, 2 or 3, more preferably 1; or preferably ● is

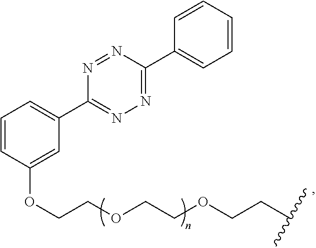

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably ● is

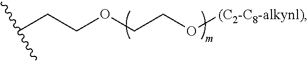

more preferably

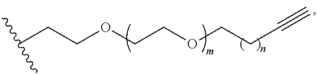

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; wherein optionally a linker is arranged between ● and Q. Accordingly, ● may be ethyl. ● may be C₁-C₈-alkyl optionally substituted with (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. ● may be C₁-C₈-alkyl optionally substituted with hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. Preferably ● is

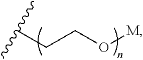

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4. ● may be a fluorophore. ● may be C₁-C₈-alkyl optionally substituted with a fluorophore. Preferably, ● is

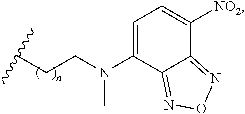

wherein n is 1, 2, 3, 4, 5, 6, 7, 8, 9 or 10, more preferably 4, 5 or 6, still more preferably 5. Preferably, ● is

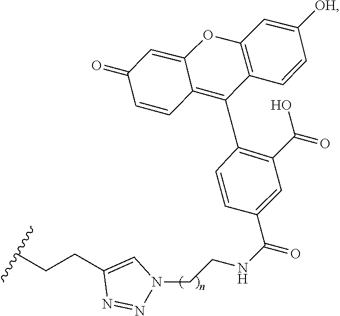

wherein n is 1, 2, 3, 4, 5, 6, 7, 8, 9 or 10, preferably 3, 4 or 5, still more preferably 4. R₁ may be C₂-C₈-alkynyl. Preferably, ● is

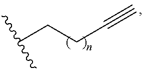

wherein n is 1, 2, 3, 4, or 5, preferably 1, 2 or 3, more preferably 1. Preferably, ● is

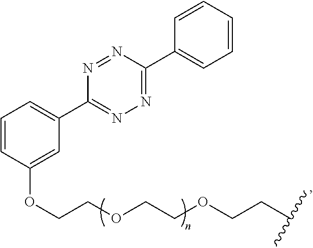

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2. Preferably, ● is

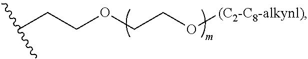

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3. More preferably, ● is

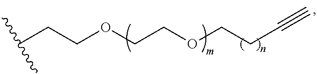

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1. Optionally, in any one of these embodiments a linker may be arranged between ● and Q.

Preferably, in any one of the compounds of formula (IV*) or the conjugates of an antibody molecule, ● may be selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably ● is

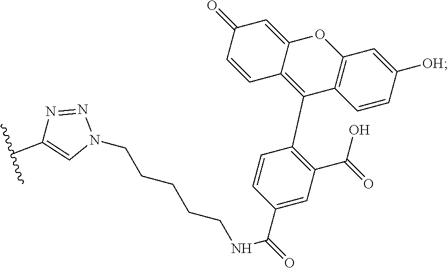 [SMILES: *c1cn(CCCCCNC(=O)c2ccc(-c3c4ccc(=O)cc-4oc4cc(O)ccc34)c(C(=O)O)c2)nn1]

or still more preferably ● is

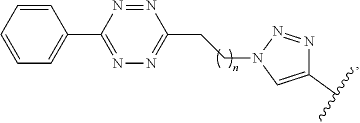

wherein n is 1, 2, 3, 4, 5, 6, 7, 8 or 9, preferably 1, 2 or 3, more preferably 1; or preferably ● is

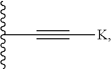

wherein K is H or C₁-C₈-alkyl, preferably K is H; wherein optionally a linker is arranged between ● and Q. Accordingly, ● may be optionally substituted aryl. Preferably, ● is optionally substituted phenyl. More preferably, ● is unsubstituted phenyl. ● may be optionally substituted heteroaryl. Preferably, ● is optionally substituted triazolyl. More preferably, ● is triazolyl substituted with optionally substituted C₁-C₈-alkyl. ● may be a fluorophore. More preferably, is triazolyl substituted with a fluorophore. Still more preferably, ● is

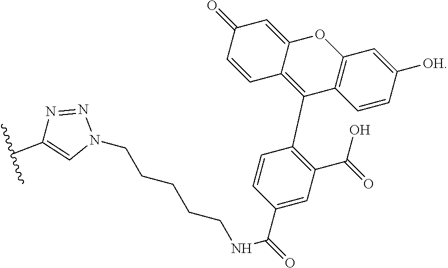 [SMILES: *c1cn(CCCCCNC(=O)c2ccc(-c3c4ccc(=O)cc-4oc4cc(O)ccc34)c(C(=O)O)c2)nn1]

Still more preferably, ● is

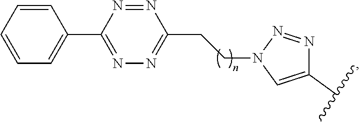

wherein n is 1, 2, 3, 4, 5, 6, 7, 8 or 9, preferably 1, 2 or 3, more preferably 1. Preferably ● is

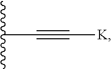

wherein K is H or C₁-C₈-alkyl, preferably H or C₁-C₆-alkyl, more preferably H or C₁-C₄-alkyl, still more preferably H or C₁-C₂-alkyl; even more preferably K is H. Optionally, in any one of these embodiments a linker may be arranged between ● and Q.

Moreover, also compounds and conjugates of an antibody molecule provided herein as examples in the example section for compounds of formula (I), (III), (IIIa), (IV), (IV*) and conjugates of an antibody molecule are preferred.

The skilled person understands that embodiments according to the invention can be combined with each other with the proviso that a combination which would contravene any natural law is excluded.

Any embodiment, feature, definition, or the like described herein with reference to any method also applies to any compound and/or conjugate of an antibody molecule mutatis mutandis. In the same manner, any embodiment, feature, definition or the like described herein with reference to any compound and/or conjugate of an antibody molecule applies to any method described herein mutatis mutandis.

Method for Sulfhydryl-/Cysteine-Specific Proteome-Wide Profiling of a Sample, and Method for Sulfhydryl-/Cysteine-Specific Isolation of Proteins from a Sample

The compounds of the present invention may also be used for a sulfhydryl-/cysteine-specific proteome-wide profiling of a sample. In such a method, the sample is incubated with a compound of formula (I) according to the present invention, wherein ● is a detectable label. Said compound is conjugated with or comprises a detectable label. Thereby, sulfhydryl-comprising proteins, in particular cysteine-comprising proteins, in the sample are conjugated with the detectable label (via the compound according to formula (I), wherein ● is a detectable label). After that, the proteins comprised in the sample (both labeled and unlabeled) may be enriched by precipitation, e.g., by using ice-cold acetone. After the optional precipitation step, the sulfhydryl-containing proteins, in particular cysteine-containing proteins, of the sample can be enriched based on the detectable label. Finally, the enriched and isolated conjugated proteins can be subjected to an enzymatic digestion, e.g., by trypsin, chymotrypsin, Lys-C, Lys-N, Asp-N, Glu-C and/or Arg-C. To analyze the proteome, the conjugated proteins or the digested proteins can be analyzed by mass spectrometry, e.g., LC-MS/MS.

Accordingly, the present invention also relates to a method for sulfhydryl-specific, in particular cysteine-specific, proteome-wide profiling of a sample, the method comprising:
(a) Incubating the sample with a compound of formula (I), wherein 4 represents a detectable label, thereby specifically conjugating proteins, which comprise a sulfhydryl group, in particular cysteine, to the label;
(b) Optionally enriching the proteins comprised in the sample by precipitation;
(c) Optionally enriching the proteins conjugated with the detectable label, preferably by contacting them with solid phase such as a bead reversibly binding to the detectable label;
(d) Analyzing the conjugated proteins.
   The detectable label may be, e.g., desthiobiotin.
   Analyzing the conjugated proteins may be carried out, e.g., by mass spectrometry. Mass spectrometry may be, e.g., LC-MS/MS. Analyzing the conjugated proteins may also include digestion, e.g., tryptic digestion, in particular before mass spectrometry. Accordingly, analyzing the conjugated proteins may be carried out, e.g., by (tryptic) digestion followed by LC-MS/MS.
   In one embodiment, step (c) comprises:
(1) Contacting the sample obtained in step (a) or (b) with a solid phase, the solid phase comprising a ligand reversibly binding to the detectable label, thereby reversibly immobilizing the proteins, which have been conjugated with the label, on the solid phase;
(2) Separating the solid phase from the remainder of the sample;
(3) Disrupting the reversible bond between the ligand of the solid phase and the label of the conjugated proteins, e.g., by displacing the label with a competitor such as biotin.
   The ligand reversibly binding to the detectable label may be, e.g., streptavidin or avidin.
   Disrupting the reversible bond between the ligand of the solid phase and the label of the conjugated proteins may be carried out, e.g., by displacing the label with a competitor, such as e.g. biotin.

The present invention is also suitable for sulfhydryl-/cysteine-specific isolation of proteins from a sample. Thereby, all sulfhydryl-comprising proteins, in particular cysteine-comprising proteins, can be isolated from a sample. Accordingly, the present invention also relates to a method for isolating sulfhydryl-comprising proteins, in particular cysteine-comprising proteins, from a sample, the method comprising:
(a) Incubating the sample with a compound of formula (I), wherein ● represents a detectable label, thereby specifically conjugating proteins, which comprise a sulfhydryl group/cysteine, to the label; and
(b) Enriching the proteins conjugated with the detectable label.
   The detectable label may be, e.g., desthiobiotin.
   Preferably, enriching the proteins conjugated with the detectable label may be carried out by contacting them with a bead reversibly binding to the detectable label.
   In one embodiment, step (b) comprises
(1) Contacting the sample obtained in step (a) with a solid phase, the solid phase comprising a ligand reversibly binding to the detectable label, thereby reversibly immobilizing the proteins, which have been conjugated with the label, on the solid phase;
(2) Separating the solid phase from the remainder of the sample;
(3) Disrupting the reversible bond between the ligand of the solid phase and the label of the conjugated proteins.
   The ligand reversibly binding to the detectable label may be, e.g., streptavidin or avidin.
   Disrupting the reversible bond between the ligand of the solid phase and the label of the conjugated proteins may be carried out, e.g., by displacing the label with a competitor, such as e.g. biotin.

The compound of formula (I), wherein ● represents a detectable label, has the following structure:

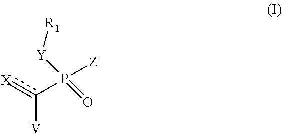

wherein Z represents a residue bound to the phosphorus via a carbon atom and comprising a group ●, wherein ● represents a detectable label; and
[symbol] , R₁, V, X and Y are as defined herein with regard to a compound of formula (I).

In some embodiments, the detectable label is desthiobiotin.

Z may be any

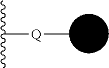

as defined herein, wherein ● represents a detectable label; and Q may be any Q as defined herein. Optionally, a linker may be arranged between ● and Q. In some embodiments, when ● represents a detectable label, Q may be a five- or six-membered heterocyclic moiety comprising 1, 2 or 3 heteroatoms independently selected from the group consisting of N, O or S. In some embodiments, Z may be

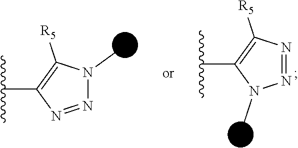

wherein R₅ is as defined herein, preferably R₅ is H. Accordingly, Z may be

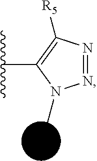

wherein Q is

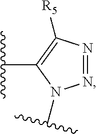

and R₅ is as defined herein, preferably R₅ is H. In preferred embodiments, when ● represents a detectable label, Z is

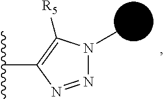

wherein Q is

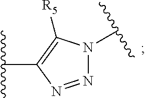

and R₅ is as defined herein, preferably R₅ is H. Optionally, in any one of these embodiments, a linker may be arranged between ● and Q.

When ● represents a detectable label, R₁ may be optionally substituted C₁-C₈-alkyl, preferably methyl, ethyl, propyl or butyl, more preferably methyl or ethyl, still more preferably ethyl. In these embodiments, the Y may be any Y as defined herein. Preferably, in any one of these embodiments, Y is O (oxygen).

In one embodiment, the compound of formula (I), wherein S represents a detectable label, is:

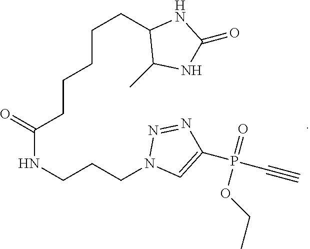 [SMILES: C#CP(=O)(OCC)c1cn(CCCNC(=O)CCCCCC2NC(=O)NC2C)nn1]

Methods for precipitation are known to a person skilled in the art and include salting out, e.g., using ammonium sulfate, isoelectric precipitation, precipitation with miscible solvents such as ethanol or methanol, non-ionic hydrophilic polymers such as dextrans or polyethylene glycols, flocculation by polyelectrolytes such as alginate, carboxymethylcellulose, polyacrylic acid, tannic acid and polyphosphates, or precipitation with polyvalent metallic ions such as Ca²⁺, Mg²⁺, Mn²⁺ or Fe²⁺.

“Analyzing” as used herein relates to any method, which results in information about the proteome, which comprises sulfhydryl or cysteine groups. Such information may relate to mass, structure, the presence or absence of a specific protein or its sulfhydryl/cysteine groups, and the like. Suitable methods include, but are not limited to, mass spectrometry, optionally after liquid (reversed-phase) chromatography (“online”-MS), immunoassays, Edman degradation, multidimensional and the like.

The term “sample”, when used herein, relates to a material or mixture of materials, typically but not necessarily in liquid form, containing one or more proteins comprising a cysteine or a sulfhydryl group. The sample may be a cell lysate. The sample of the present invention may be a biological sample. The term “biological sample” as used herein refers to a sample obtained from a subject, wherein the sample may be any biological tissue or fluid sample. Frequently, the sample will be a “clinical sample” which is a sample derived from a patient. The biological sample of the present invention may be a serum sample, a plasma sample, a urine sample, a feces sample, a saliva sample, a tear fluid sample, or a tissue extract sample. Further samples envisaged are sputum, cerebrospinal fluid, fine needle biopsy samples, peritoneal fluid, and pleural fluid, but the invention is not limited thereto. In the context of the present invention, the sample may be a saliva sample, a serum sample, a tissue sample, a blood sample, a urine sample, a lymphatic fluid sample, a nasopharyngeal wash sample, a sputum sample, a mouth swab sample, a throat swab sample, a nasal swab sample, a bronchoalveolar lavage sample, or a bronchial secretion sample. Biological samples may also include sections of tissues such as frozen sections taken for histological purposes. Such samples include, for example, whole blood, serum, etc. Preferably, a sample is a sample that includes extracellular material or liquid. Biological samples can be analyzed directly or they may be subject to some preparation prior to use in methods or kits of the present invention. Such preparation can include, but is not limited to, suspension/dilution of the sample in water or an appropriate buffer or removal of cellular debris, e.g. by centrifugation, precipitation, or selection of particular fractions of the sample before analysis.

“Enriching” as used herein describes the process of increasing the amount of the bead/complex/substance in a mixture. Typically, the sample is contacted with a solid phase such as a bead reversibly binding to the detectable label. The solid phase may then be separated from the remaining sample or supernatant. The solid phase may be washed. Finally, the proteins conjugated to the detectable label may be eluted from the solid phase, e.g., by adding a competitor as described herein.

The solid phase may be used in a batch method or a chromatographic method. Accordingly, the method or sulfhydryl-/cysteine-specific proteome-wide profiling of a sample or for isolating sulfhydryl-/cysteine-comprising proteins from a sample described herein may be a batch method. The method or sulfhydryl-/cysteine-specific proteome-wide profiling of a sample or for isolating sulfhydryl-/cysteine-comprising proteins from a sample described herein may also be a chromatographic method. The solid phase may comprise one or more ligands reversibly binding to the detectable label.

As already mentioned, the method or sulfhydryl-/cysteine-specific proteome-wide profiling of a sample or for isolating sulfhydryl-/cysteine-comprising proteins from a sample described herein may be practiced as part of fluid chromatography, typically a liquid chromatography. Any material may be employed as chromatography matrix in the context of the invention, as long as the material is suitable for the chromatographic isolation of the chosen biological entity such as proteins. The chromatography matrix corresponds to the solid phase in the context of the method or sulfhydryl-/cysteine-specific proteome-wide profiling of a sample or for isolating sulfhydryl-/cysteine-comprising proteins from a sample described herein. A chromatography matrix as used in the method or sulfhydryl-/cysteine-specific proteome-wide profiling of a sample or for isolating sulfhydryl-/cysteine-comprising proteins from a sample described herein typically remains in a predefined location, typically in a predefined position, whereas the location of the sample to be separated and of components included therein is being altered, i.e. the chromatography matrix can also be seen as a stationary phase. As an illustrative example, if packed-bed chromatography columns are employed, the solid phase is generally confined between the bottom of the column and the flow adapter. Where chromatography is carried out as expanded bed adsorption, the resin becomes fluidized in use, and beads employed arrange in the form of a concentration gradient, individual beads taking a position where their sedimentation velocity matches the upward liquid flow velocity. The chromatography matrix is thus a “stationary phase” (corresponding to the “solid phase” used in the context of the present invention) in line with the common understanding of the person skilled in the art in that the stationary phase is that part of a chromatographic system through which the mobile phase flows and where components included in the liquid phase are being disseminated between the phases.

If beads are employed, in column chromatography beads are commonly rather uniform in size, whereas in expanded bed adsorption beads are variable in size, typically ranging from about 50 to about 400 mm. In this regard, it is noted that particles such as freely moveable magnetic beads that are added to a liquid sample, mixed with the sample and are then removed from the sample, for example, by discarding the supernatant (liquid) while holding the beads temporarily in place (for example, by an external magnetic or by centrifugation) are in one embodiment not a solid phase as used herein. However, the method or sulfhydryl-/cysteine-specific proteome-wide profiling of a sample or for isolating sulfhydryl-/cysteine-comprising proteins from a sample described herein can also be practiced in a batch mode. In such a method (magnetic) beads can be added to a sample containing the cysteine-/sulfhydryl-containing proteins for immobilization of the proteins on such beads, and the beads are then separated from the sample, for example by temporarily holding the beads in place, while discarding the supernatant Such a batch method is also a method according to the invention.

Typically, the respective chromatography matrix has the form of a solid or semi-solid phase, whereas the sample is a fluid phase. The mobile phase used to achieve separation is likewise a fluid phase. The chromatography matrix can be a particulate material (of any suitable size and shape) or a monolithic chromatography material, including a paper substrate or membrane. Thus, the chromatography can for example be column chromatography. In some embodiments the chromatography may be planar chromatography. In some embodiments the chromatography may be expanded bed chromatography. If a particulate matrix material is used in column chromatography, the particulate matrix material may, for example, have a mean particle size of about 5 μm to about 200 μm, or from about 5 μm to about 400 μm, or from about 5 μm to about 600 μm. As explained in detail the following, the chromatography matrix may, for example, be or include a polymeric resin or a metal oxide or a metalloid oxide. If planar chromatography is used, the matrix material may be any material suitable for planar chromatography, such as conventional cellulose-based or organic polymer based membranes (for example, a paper membrane, a nitrocellulose membrane or a polyvinylidene difluoride (PVDF) membrane) or silica coated glass plates. In one embodiment, the chromatography matrix/solid phase is a non-magnetic material or non-magnetisable material.

Non-magnetic or non-magnetisable chromatography solid phases that are used in the art, and that are also suitable in a the method or sulfhydryl-/cysteine-specific proteome-wide profiling of a sample or for isolating sulfhydryl-/cysteine-comprising proteins from a sample described herein described herein, include derivatized silica or a crosslinked gel. A crosslinked gel (which is typically manufactured in a bead form) may be based on a natural polymer, i.e. on a polymer class that occurs in nature. For example, a natural polymer on which a chromatography solid phase is based is a polysaccharide. A respective polysaccharide is generally crosslinked. An example of a polysaccharide matrix is an agarose gel (for example, Superflow™ agarose or a Sepharose® material such as Superflow™ Sepharose® that are commercially available in different bead and pore sizes) or a gel of crosslinked dextran(s). A further illustrative example is a particulate cross-linked agarose matrix, to which dextran is covalentiy bonded, that is commercially available (in various bead sizes and with various pore sizes) as Sephadex® or Superdex®, both available from GE Healthcare. Another illustrative example of such a chromatography material is Sephacryl® which is also available in different bead and pore sizes from GE Healthcare.

A crosslinked gel may also be based on a synthetic polymer, i.e. on a polymer class that does not occur in nature. Usually such a synthetic polymer on which a chromatography solid phase for cell separation is based is a polymer that has polar monomer units, and which is therefore in itself polar. Such a polar polymer is hydrophilic. Hydrophilic (“water-loving”) molecules, also termed lipophobic (“fat hating”), contain moieties that can form dipole-dipole interactions with water molecules. Hydrophobic (“water hating”) molecules, also termed lipophilic, have a tendency to separate from water.

Illustrative examples of suitable synthetic polymers are polyacrylamide(s), a styrene-divinylbenzene gel and a copolymer of an acrylate and a diol or of an acrylamide and a diol. An illustrative example is a polymethacrylate gel, commercially available as a Fractogel®. A further example is a copolymer of ethylene glycol and methacrylate, commercially available as a Toyopear®. In some embodiments a chromatography solid phase may also include natural and synthetic polymer components, such as a composite matrix or a composite or a co-polymer of a polysaccharide and agarose, e.g. a polyacrylamide/agarose composite, or of a polysaccharide and N,N′-methylenebisacrylamide. An illustrative example of a copolymer of a dextran and N,N′-methylenebisacryl-amide is the above-mentioned Sephacryl® series of material. A derivatized silica may include silica particles that are coupled to a synthetic or to a natural polymer. Examples of such embodiments include, but are not limited to, polysaccharide grafted silica, polyvinyl-pyrrolidone grafted silica, polyethylene oxide grafted silica, poly(2-hydroxyethylaspartamide) silica and poly(N-isopropylacrylamide) grafted silica.

A solid phase such as a chromatography matrix employed in a the method or sulfhydryl-/cysteine-specific proteome-wide profiling of a sample or for isolating sulfhydryl-/cysteine-comprising proteins from a sample described herein may also include magnetically attractable particles. Also such respective magnetically attractable particles may include a ligand reversibly binding to the detectable label comprised in the solid phase. Magnetically attractable particles may contain diamagnetic, ferromagnetic, paramagnetic or superparamagnetic material. Superparamagnetic material responds to a magnetic field with an induced magnetic field without a resulting permanent magnetization. Magnetic particles based on iron oxide are for example commercially available as Dynabeads® from Dynal Biotech, as magnetic MicroBeads from Miltenyi Biotec, as magnetic porous glass beads from CPG Inc., as well as from various other sources, such as Roche Applied Science, BIOCLON, BioSource International Inc., micromod, AMBION, Merck, Bangs Laboratories, Polysciences, or Novagen Inc., to name only a few. Magnetic nanoparticles based on superparamagnetic Co and FeCo, as well as ferromagnetic Co nanocrystals have been described, for example by Hutten, A. et al. (J. Biotech. (2004), 112, 47-63). In some embodiments a chromatography matrix employed in a method disclosed herein is void of any magnetically attractable matter.

A “proteome” as used herein relates to the entire set of proteins that is, or can be, expressed by a genome, cell, tissue, or organism at a certain time.

The non-covalent bond that is formed between the detectable label (of the conjugated protein) and the ligand of the solid phase (reversibly binding to the detectable label) may be of any desired strength and affinity, as long as it is disruptable or reversible under the conditions under which the methods of the invention is performed. The dissociation constant (KD) of the binding between the detectable label (of the conjugated protein) and the ligand of the solid phase may have a value in the range from about 10⁻² M to about 10⁻¹³ M. Thus, this reversible bond can, for example, have a K_{D} from about 10⁻² M to about 10⁻¹³ M, or from about 10⁻³ M to about 10⁻¹² M or from about 10⁻⁴ M to about 10⁻¹¹M, or from about 10−5 M to about 10⁻¹⁰ M. The K_{D} of this bond as well as the K_{D}, k_{off} and kₒₙ, rate of the bond formed between the detectable label (of the conjugated protein) and the ligand of the solid phase (reversibly binding to the detectable label) can be determined by any suitable means, for example, by fluorescence titration, equilibrium dialysis or surface plasmon resonance. The conjugated protein may include at least one, including two, three or more, detectable labels and the solid phase may include at least one, at least two, such as three, four, five, six, seven, eight or more ligands reversibly binding to the detectable label.

As already described herein, the binding of the detectable label (of the conjugated protein) and the ligand of the solid phase (reversibly binding to the detectable label) reversible. Disrupting (displacing) the reversible binding of the detectable label (of the conjugated protein) and the ligand of the solid phase (reversibly binding to the detectable label) can be achieved by contacting the sample with a composition comprising a substance capable of reversing the bond between the detectable label (of the conjugated protein) and the ligand of the solid phase (reversibly binding to the detectable label) (“competitor” as used herein). For example, the competitor is a free binding partner and/or is a competition agent (e.g. a biotin, a biotin analog, a biologically active fragment thereof). In some embodiments, the methods of the invention include after applying a competitor to disrupt (displace) the bond between the detectable label (of the conjugated protein) and the ligand of the solid phase (reversibly binding to the detectable label), thereby recovering the selected conjugated proteins from the solid phase or in other words, eluting a fraction of the conjugated proteins from the solid phase. The choice of the competitor depends on the particular detectable label (of the conjugated protein) and the ligand of the solid phase (reversibly binding to the detectable label). In some embodiments, the ligand of the solid phase (reversibly binding to the detectable label) is a streptavidin mutein (e.g. Strep-Tactin®) for recognition of a streptavidin binding peptide (e.g. Strep-tag® or a Twin-Strep-tag®) comprised in the detectable label and the competitor is biotin or a biotin analog.

The term “competitor” or “competition reagent”—both terms can be used interchangeably—as used herein refers to any reagent or condition that is able to reduce, interfere with or abrogate the formation of a complex between a pair of binding agents or moieties, such as a the detectable label (of the conjugated protein) and the ligand of the solid phase (reversibly binding to the detectable label). The term “competition” is meant to refer any interference with binding, regardless of the nature of such interference. Such interference may in some embodiments also be a non-competitive binding to a certain binding site. An example of such a competition mechanism is the metal chelation by a chelating reagent such as EDTA or EGTA, when the reversibly bond is mediated by complexed metal ions such as Ca²⁺, Ni²⁺, Co²⁺, or Zn²⁺. This mechanism applies for binding pairs such as calmodulin and calmodulin binding peptides that bind in the presence of Ca²⁺ or for binding pairs that are used in Immobilized Metal-chelate Affinity Chromatography (IMAC). In some embodiments, a competition reagent may have a binding site that is capable of specifically binding to the binding site included on one of the binding partners, e.g. the detectable label (of the conjugated protein) and the ligand of the solid phase (reversibly binding to the detectable label). It is also possible that the entire competition reagent is capable of specifically binding to the binding site included on one of these binding partners. In some embodiments competition is provided by a change in pH or the salt strength of a buffer and the competition reagent is then either an increased or decreased pH or salt strength. A change in pH can, for example, be used for displacing/disrupting the binding of streptavidin to a streptavidin binding peptide or for displacing/disrupting the binding between protein A or protein G and an antibody Fc domain. Preferably, the competitor is Biotin or a derivative thereof, more preferably Biotin. The competitor may be a reducing agent such as Dithiothreitol (DTT), LiAlH₄, NaBH₄, dithionates, thiosulfates, iodides, ascorbic acid or the like, preferably DTT.

In one embodiment, the ligand reversibly binding to the detectable label is an antibody or a fragment thereof. In such an embodiment, the disruption of the reversible bond can be carried out by a change in pH.

As described herein, the compound according to formula (I) as described herein may comprise a detectable label. Label and detectable label are used interchangeably herein. Preferred labels include, but are not limited to, an enzyme, a radioisotope, a fluorescent protein, a fluorescent dye, a bioluminescent label or a tag (e.g., biotin or desthiobiotin). The detectable labels can be any of the various types used currently in the field of in vitro diagnostics, including particulate labels including metals such as colloidal gold, isotopes, chromophores including fluorescent markers, biotin, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, polyHRP, alkaline phosphatase and the like, preferably horseradish peroxidase. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-star® or other luminescent substrates well-known to those in the art, for example the chelates of suitable lanthanides such as Terbium(II) and Europium(II). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice. Accordingly, the label may be detected based on optical, fluorescent, luminescent, electrochemiluminescent and/or multi-analyte profiling (xMAP) readouts or means. The label may be detected by optical means such as absorption at a particular wavelength or inspection by the naked eye. The label may be detected by fluorescent means such as determining the emission of a fluorophore at a specific wavelength after excitation at a different, typically shorter, wave length. The label may be detected by electro chemiluminescent means, e.g., making of use the commercially available ELECSYS system by Roche. The label may be detected by multi-analyte profiling (xMAP), e.g., as described in WO 2007/075891.

A “tag” as used herein may include, but is not limited to, affinity tags that are appended to proteins so that they can be purified from their crude biological source using an affinity technique such as chitin binding protein (CBP), maltose binding protein (MBP), Strep-tag and glutathione-S-transferase (GST) or the poly(His) tag is a widely used protein tag, which binds to metal matrices; chromatography tags that are used to alter chromatographic properties of the protein to afford different resolution across a particular separation technique such as FLAG-tag; epitope tags that are short peptide sequences which are chosen because high-affinity antibodies can be reliably produced in many different species such as ALFA-tag, V5-tag, Myc-tag, HA-tag, Spot-tag, T7-tag and NE-tag; fluorescence tags that are used to give visual readout on a protein such as GFP and its variants; protein tags that may allow specific enzymatic modification (such as biotinylation by biotin ligase) or chemical modification (such as reaction with FIAsH-EDT2 for fluorescence imaging).

### Synthesis of Compounds and Conjugates of an Antibody Molecule

The following section provides some general features of the synthesis of compounds of formulae I, III, IIIa, IV, IV* and the conjugate of an antibody molecule. In general, a person skilled in the art knows to select suitable starting materials and reaction conditions for carrying out such synthesis. Further details are given in the Example section below. Any variables, e.g. R₁, R², R₃, R₄, R₅, V, X, Y, Z, [symbol] , [symbol] , ●, [symbol] and any others, are as defined throughout this specification, if not noted otherwise.

### Synthesis of Compounds of Formula (IV) or (IV*)

Compounds of formula (IV), wherein Y is O or S, or compounds of formula (IV*) wherein ▴ is —Y—R₁ and Y is O or S, can be prepared, e.g., from a dialkyl-phosphoramidous dihalide, e.g. commercially available diethyl-phosphoramidous dichloride, as indicated in the following scheme:

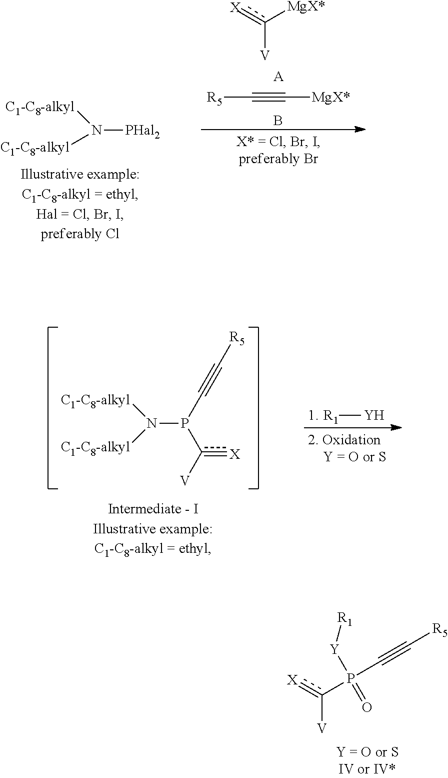

Accordingly, the dialkyl-phosphoramidous dihalide, e.g. diethyl-phosphoramidous dichloride, can be reacted with a Grignard compound A comprising an X group, and a Grignard compound B comprising an R₅ group to form Intermediate-I. A person skilled in the art will select Grignard compounds A and B in order to obtain the desired substitution pattern. In some embodiments, Grignard compounds A and B are the same (in other words, only one Grignard compound is used), e.g., in A the [symbol] is a triple bond, V is absent, X is R₃C, R₃ and R₅ are the same, preferably R₃ and R₅ are hydrogen. Then, when A and B are the same, the dialkyl-phosphoramidous dihalide, e.g. diethyl-phosphoramidous dichloride, can be reacted with 2 to 3, e.g. 2.5, equivalents of the Grignard compound to give an Intermediate-I which comprises two identical ethynyl substitutents. Alternatively, when Grignard compounds A and B are different, the dialkyl-phosphoramidous dihalide, e.g. diethyl-phosphoramidous dichloride, can be reacted, in sequential order, with about 1 eq. of Grignard compound A, and thereafter with about 1 eq. of a Grignard compound B, to obtain a respective Intermediate-1 having different substituents introduced by the Grignard compounds. Grignard compounds A and B can be reacted in any sequential order, i.e. first reacting A and then B, or first reacting B and then A. A person skilled in the art will readily select a suitable sequential order. Reaction of the dialkyl-phosphoramidous dihalide, e.g. diethyl-phosphoramidous dichloride, with the Grignard compound(s) to give Intermediate-I can be effected applying typical known conditions for Grignard reactions, e.g. using tetrahydrofuran (THF) of diethyl ether as solvent, at a low temperature below −50° C., and then warming the reaction; e.g. the temperature range may be between −100° C. and +50° C.; more specifically, the reaction may be carried out at about −78° C. and then warming to room temperature. A person skilled in the art knows to select suitable reaction conditions. Preferably, the reaction with the Grignard compound is carried out under an inert gas, such as argon. In many cases it is not necessary to isolate Intermediate-I; rather, Intermediate-I can be used in subsequent reactions, e.g. in a reaction with R₁—YH, without isolation. Thus, for reacting with R₁—YH, the mixture comprising Intermediate-I can be cooled again, and R₁—YH wherein Y is O (i.e. an alcohol) or Y is S (i.e. a thiol) can be added in a suitable solvent, e.g. acetonitrile. The reaction with R₁—YH can be carried out at low temperature, e.g. below −50° C. and preferably at −78° C. and then warming, e.g. to room temperature, or even up to +50° C. Preferably, the reaction with R₁—YH is carried out in the presence of tetrazole. Preferably, the reaction with R₁—YH can be carried out under an inert gas, such as argon. Optionally, after reaction with R₁—YH, the mixture can be worked-up using procedures commonly known to a person skilled in the art, e.g. including extraction, and the crude product may be obtained after evaporation of the solvent. Optionally, if desired, the crude product may be further purified, e.g. using silica gel chromatography. In order to obtain a compound of formula (IV) or (IV*), the compound is further oxidized using a suitable oxidant. Various suitable oxidants may be used, such as e.g. tert-butylhydroperoxide (tBu-OOH), meta-chloroperoxybenzoic acid (mCPBA), hydrogen peroxide (H₂O₂), iodine (I₂), potassium peroxymonosulphate, or oxygen (O₂), e.g. oxygen from air. The skilled person will readily determine a suitable oxidant. Preferably, H₂O₂ is used as oxidant. For example, the oxidation may be carried out using H₂O₂ in water/acetonitrile as solvent. The oxidation may be carried out at a suitable temperature, e.g. between 0° C. and 50° C., e.g. at room temperature. After oxidation, the compound of formula (IV)/formula (IV*) can be isolated using work-up procedures and/or purification methods known to a person skilled in the art. For example, after oxidation, the mixture can be lyophilized to provide the compound of formula (IV)/formula (IV*).

Compounds of formula (IV) wherein Y is NR², O or S, in particular wherein Y is NR², or formula (IV*) wherein ▴ is —Y—R₁ and Y is NR², O or S, in particular wherein Y is NR², can be also prepared via Intermediate-I, as shown in the following scheme:

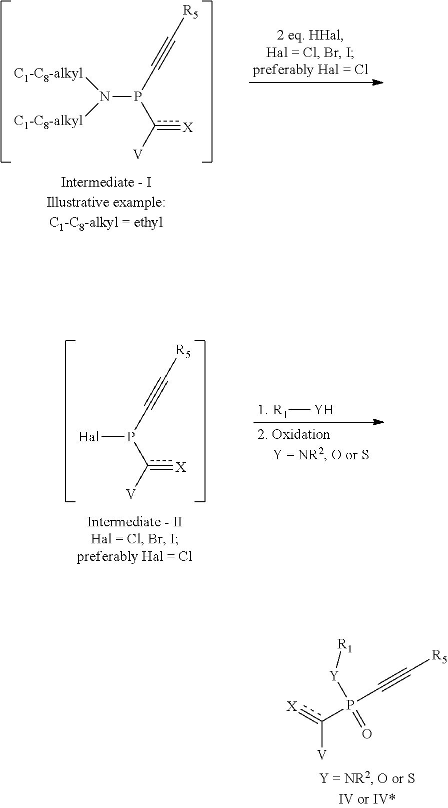

Accordingly, Intermediate-I, which again does not need to be isolated after the Grignard reaction but can be rather used in a subsequent reaction without isolation, is reacted with about two equivalents of an acid, e.g. a hydrohalic acid such as HCl, HBr or HI, preferably HCl, to obtain Intermediate-II (see, e.g., Van Assema et al. (2007), J. Organomet. Chem., which is incorporated herein by reference in its entirety). The reaction with the hydrohalic acid can be carried out in a suitable solvent, e.g. the reaction can be carried out using hydrochloric acid in diethyl ether. Suitable reaction temperatures are readily selected by a person skilled in the art and can be, e.g., in the range of from −20° C. to +20° C.; in particular, the reaction can be carried out 0° C. Intermediate-II is typically not isolated, and subjected to the reaction with R₁—YH wherein Y is NR², i.e. an amine. It is also possible to react Intermediate-II with R₁—YH wherein Y is O or S. Thus, for reacting with R₁—YH, the mixture comprising Intermediate-II can be cooled again, and R₁—YH wherein Y is NR₂ (or O or S) can be added in a suitable solvent, e.g. diethyl ether, tetrahydrofuran or acetonitrile. The reaction with R₁—YH can be carried out at low temperature, e.g. below −50° C. and preferably at −78° C. and then warming, e.g. to room temperature, or even up to +50° C. Preferably, the reaction with R₁—YH is carried out in the presence of pyridine. Preferably, the reaction with R₁—YH can be carried out under an inert gas, such as argon. Optionally, after reaction with R₁—YH, the mixture can be worked-up using procedures commonly known to a person skilled in the art, e.g. including extraction, and the crude product may be obtained after evaporation of the solvent. Optionally, if desired, the crude product may be further purified, e.g. using silica gel chromatography. In order to obtain a compound of formula (IV) or (IV*), the compound is further oxidized using a suitable oxidant, as described herein. Various suitable oxidants may be used, such as e.g. tert-butylhydroperoxide (tBu-OOH), meta-chloroperoxybenzoic acid (mCPBA), hydrogen peroxide (H₂O₂), iodine (I₂), potassium peroxymonosulphate, or oxygen (O₂), e.g. oxygen from air. The skilled person will readily determine a suitable oxidant. Preferably, H₂O₂ is used as oxidant. For example, the oxidation may be carried out using H₂O₂ in water/acetonitrile as solvent. The oxidation may be carried out at a suitable temperature, e.g. between 0° C. and 50° C., e.g. at room temperature. After oxidation, the compound of formula (IV)/formula (IV*) can isolated using work-up procedures and/or purification methods known to a person skilled in the art. For example, after oxidation, the mixture can be lyophilized to provide the compound of formula (IV)/formula (IV*).

Compounds of formula (IV) wherein Y is a bond, or compounds of formula (IV*) wherein ▴ is —Y—R₁ and Y is a bond, or compounds of formula (IV*) wherein ▴ is Z and Z represents a residue bound to the phosphorus via a carbon atom and comprising a group ●, wherein ● represents an optionally substituted aliphatic or optionally substituted aromatic residue, can be also prepared via Intermediate-II, as shown in the following scheme:

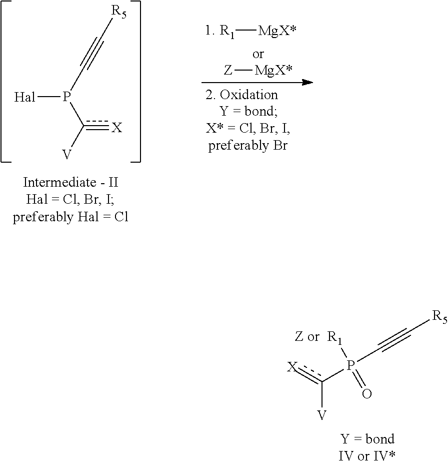

Intermediate-II can be subjected to a reaction, e.g., with a Grignard compound, in order to form a compound of formula IV or formula IV* wherein Y is a bond. Reaction of Intermediate-II with R₁—MgX or Z—MgX* can be effected applying typical conditions for Grignard reactions, e.g. conditions described above using tetrahydrofuran or diethyl ether as solvent, at a low temperature below −50° C. and +50° C.; more specifically, the reaction may be carried out at about −78° C. and then warming to room temperature. A person skilled in the art knows to select suitable reaction conditions. Preferably, the reaction with the Grignard reagent reaction is carried out under an inert gas, such as argon. Optionally, after reaction with R₁—MgX* or Z—MgX*, the mixture can be worked-up using procedures commonly known to a person skilled in the art, e.g. including extraction, and the crude product may be obtained after evaporation of the solvent. Optionally, if desired, the crude product may be further purified, e.g. using silica gel chromatography. In order to obtain a compound of formula (IV) or (IV*), the compound is further oxidized using a suitable oxidant, as described herein. Various suitable oxidants may be used, such as e.g. tert-butylhydroperoxide (tBu-OOH), meta-chloroperoxybenzoic acid (mCPBA), hydrogen peroxide (H₂O₂), iodine (I₂), potassium peroxymonosulphate, or oxygen (O₂), e.g. oxygen from air. The skilled person will readily determine a suitable oxidant. Preferably, H₂O₂ is used as oxidant. For example, the oxidation may be carried out using H₂O₂ in water/acetonitrile as solvent. The oxidation may be carried out at a suitable temperature, e.g. between 0° C. and 50° C., e.g. at room temperature. After oxidation, the compound of formula (IV)/formula (IV*) can be isolated using work-up procedures and/or purification methods known to a person skilled in the art. For example, after oxidation, the mixture can be lyophilized to provide the compound of formula (IV) or formula (IV*).

Compounds of formula IV wherein Y is O, S or NR², or compounds of formula IV* wherein ▴ is —Y—R₁ and Y is O, S or NR² can be, e.g., also prepared according to the following Scheme:

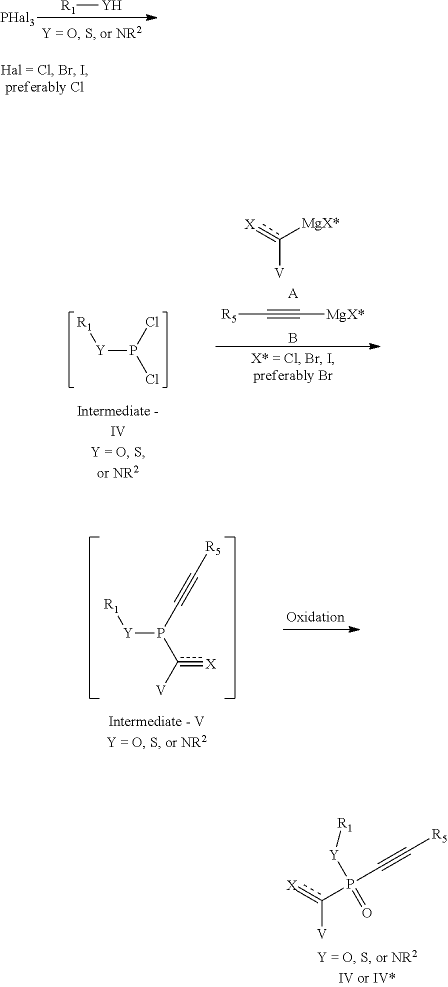

For example, a phosphorus trihalide, preferably PCl₃, can be reacted with R₁—YH, wherein Y is O, S or NR², in a suitable solvent, such as e.g. diethyl ether or tetrahydrofuran, at a low temperature below −10° C., and then warming the reaction mixture; for example, the temperature range may be between −50° C. and +50° C.; more specifically, the reaction may be carried out at about −40° C. or −30° C. and then warming to room temperature. Preferably, the reaction of the phosphorus trihalide with the alcohol is carried out in presence of a weak base, such as e.g. an amine base like triethylamine. Preferably, the reaction is carried out under an inert gas, such as argon. Intermediate-IV can be typically used in the subsequent Grignard reaction without isolation. Optionally, the mixture may be purified, e.g. by celite filtration. Then, Intermediate-IV can be reacted with a Grignard compound A comprising an X group, and a Grignard compound B comprising an R₅ group to form Intermediate-V. A person skilled in the art will select Grignard compounds A and B in order to obtain the desired substitution pattern. In some embodiments, Grignard compounds A and B are the same (in other words, only one Grignard compound is used), e.g., in A the [symbol] is a triple bond, V is absent, X is R₃C, R₃ and R₅ are the same, preferably R₃ and R₅ are hydrogen. Then, when A and B are the same, the dialkyl-phosphoramidous dihalide, e.g. diethyl-phosphoramidous dichloride, can be reacted with 2 to 3, e.g. 2.5, equivalents of the Grignard compound to give an Intermediate-V which comprises two identical ethynyl substituents. Alternatively, when Grignard compounds A and B are different, the dialkyl-phosphoramidous dihalide, e.g. diethyl-phosphoramidous dichloride, can be reacted, in sequential order, with about 1 eq. of Grignard compound A, and thereafter with about 1 eq. of a Grignard compound B, to obtain a respective Intermediate-V having different substituents introduced by the Grignard compounds. Grignard compounds A and B can be reacted in any sequential order, i.e. first reacting A and then B, or first reacting B and then A. A person skilled in the art will readily select a suitable sequential order. Reaction of Intermediate-IV with the Grignard compound to give Intermediate-V can be effected applying typical known conditions for Grignard reactions, e.g. conditions as described above using tetrahydrofuran (THF) or diethyl ether as solvent, at a low temperature below −50° C., and then warming the reaction; e.g. the temperature range may be between −100° C. and +50° C.; more specifically, the reaction may be carried out at about −78° C. and then warming to room temperature. A person skilled in the art knows to select suitable reaction conditions. Preferably, the reaction with the Grignard compound is carried out under an inert gas, such as argon. In many cases it is not necessary to isolate Intermediate-V; rather, Intermediate-V can be used in the following oxidation without isolation. Accordingly, in order to obtain a compound of formula (IV) or (IV*), Intermediate-V is further oxidized using a suitable oxidant. Various suitable oxidants may be used, such as e.g. tert-butylhydroperoxide (tBu-OOH), meta-chloroperoxybenzoic acid (mCPBA), hydrogen peroxide (H₂O₂), iodine (I₂), potassium peroxymonosulphate, or oxygen (O₂), e.g. oxygen from air. The skilled person will readily determine a suitable oxidant. Preferably, H₂O₂ is used as oxidant. For example, the oxidation may be carried out using H₂O₂ in water/acetonitrile as solvent. The oxidation may be carried out at a suitable temperature, e.g. between 0° C. and 50° C., e.g. at room temperature. After oxidation, the compound of formula (IV)/formula (IV*) can be isolated using work-up procedures and/or purification methods known to a person skilled in the art. For example, after oxidation, the mixture can be lyophilized to provide the compound of formula (IV)/formula (IV*).

Further synthetic methods to provide compounds of formula (IV) or formula (IV*), wherein an aliphatic or aromatic residue is bound to the phosphorus atom via a carbon atom, are generally known to a person skilled in the art. Accordingly, compounds of formula (IV) wherein Y is a bond, or compounds of formula (IV*) wherein ▴ is —Y—R₁ and Y is a bond, or preferably compounds of formula (IV*) wherein is Z and Z represents a residue bound to the phosphorus via a carbon atom and comprising a group ●, wherein ● represents an optionally substituted aliphatic or optionally substituted aromatic residue, can be also prepared from optionally substituted phosphine oxide, as shown in the following scheme; in particular when Z is

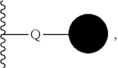

wherein [symbol] indicates the attachment point to the phosphorus and Q is a moiety comprising at least three main-chain atoms and a carbon-carbon double bond, wherein at least one of the main chain atoms is a heteroatom selected from the group consisting of S, O or N:

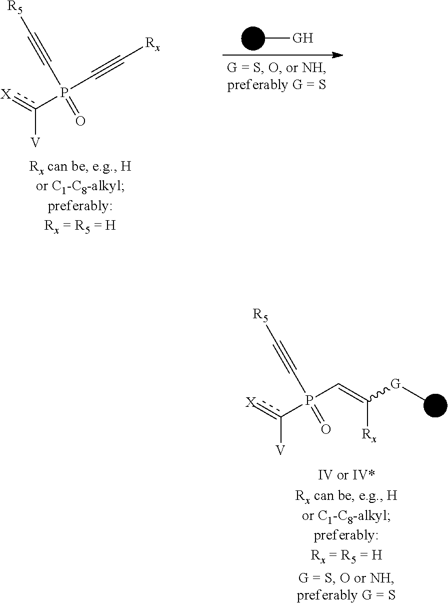

In these embodiments the compound of formula (IV) or (IV*) is formed by addition of ●-GH wherein G is S, O, or NH, to the triple bond to form a compound of formula (IV*); preferably, G is S. In some embodiments, [symbol] is a triple bond, V is absent, X is R₃C, preferably R₃, R₅ and Rₓ are the same, more preferably, R₃, R₅ and Rₓ are each hydrogen. The reaction is carried out in a suitable solvent, which is readily determined by a person skilled in the art. The solvent system can be chosen from a wide range of solvents. The solvent can be a polar aprotic solvent system such as tetrahydrofuran (THF), dimethylformamide (DMF), acetonitrile (MeCN), acetone, dimethyl sulfoxide (DMSO), ethyl acetate (EtOAc), N-ethylpyrrolidone or mixtures thereof, preferably THF, DMF, DMSO; nonpolar solvents such as hexane, toluene, benzene, 1,4-dioxane, chloroform, diethyl ether or dichloromethane (DCM), preferably DCM; polar protic solvents such as water, ethanol, isopropanol, methanol, n-butanol, preferably ethanol; or mixtures thereof. For example, the reaction may be carried out in DMF, DMSO, a DMF/water mixture, or a DMSO/water mixture. In particular, the reaction may be carried out in DMF, DMSO, a DMF/water mixture, or a DMSO/water mixture when a biomolecule, such as e.g. a protein, an antibody, a peptide, a nucleotide or an oligonucleotide, is reacted. The solvent may be also an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), or borate-containing phosphate-buffered saline. Carrying out the reaction in a buffer is preferred in case a biomolecule, such as e.g. a protein, an antibody, a peptide, a nucleotide or an oligonucleotide, is employed in the reaction. The reaction may be also carried out in a mixture of any one of the aforementioned aqueous buffers and DMF, or DMSO. Suitable solvents and buffers will be readily selected by a person skilled in the art. Preferably, the reaction is carried out under basic conditions, in particular under slightly basic conditions, e.g. at a pH of e.g. between 7.2 and 9, such as e.g. at a pH of 8 or 8.5. Such basic conditions may be established by using a suitable buffer system, such as e.g. by using any one of the buffers mentioned above. In addition or alternatively, basic conditions for the reaction may be established by using a weak base. Suitable bases are e.g. carbonates such as (NH₄)₂CO₃, Na₂CO₃, Rb₂CO₃, K₂CO₃ or Cs₂CO₃ or correlating hydrogencarbonates thereof (e.g. NaHCO₃ etc.); and weak nitrogen-containing bases such as trimethylamine Et₃N (pKa 10,76 at 25° C.). Preferably, a base with a pKₐ value within the range of 7,5 to 11,5 is used. Suitable bases will be readily selected by a person skilled in the art. The reaction temperature is not particularly limited. For example, the reaction may be carried out at temperatures in a range of from 0° C. to 60° C., of from 0° C. to 50° C., of from 0° C. to 40° C., of from 0° C. to 30° C., e.g. at room temperature, i.e. around 25° C., e.g. at around 5° C., or e.g. at physiologically relevant conditions at around 37° C. Suitable reaction conditions, including temperatures and reaction times, will be readily selected by a person skilled in the art.

Compounds of formula (IV) wherein Y is a bond, or compounds of formula (IV*) wherein ▴ is —Y—R₁ and Y is a bond, or preferably compounds of formula (IV*) wherein ▴ is Z and Z represents a residue bound to the phosphorus via a carbon atom and comprising a group ●, wherein ● represents an optionally substituted aliphatic or optionally substituted aromatic residue, can be also prepared from optionally substituted phosphine oxide as shown in the following structure (in some embodiments, [symbol] is a triple bond, V is absent, X is R₃C, preferably R₃, R₅ and Rₓ are the same, more preferably, R₃, R₅ and Rₓ are each hydrogen):

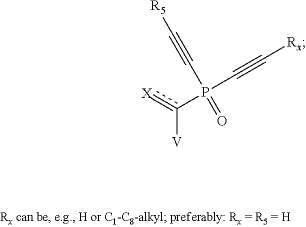

for example, such optionally substituted phosphine oxide can be used to prepare compounds where Z is

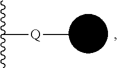

wherein [symbol] indicates the attachment point to the phosphorus and Q is a five- or six-membered heterocyclic moiety comprising 1, 2 or 3 heteroatoms independently selected from the group consisting of N, O or S. Such heterocyclic moiety can be formed, e.g., using cycloaddition reactions, as known to a person skilled in the art. For example, a six-membered heterocyclic moiety may be obtained by reacting the phosphine oxide with a suitable hetero-diene in a hetero-Diels-Alder reaction. A five-membered heterocyclic moiety may be formed by reacting the phosphine oxide with a suitable 1,3-dipole compound in a 1,3-dipolar cycloaddition (the 1,3-dipolar cycloaddition is also known as click reaction). Suitable hetero-dienes and 1,3-dipole compounds are known to a person skilled in the art and readily selected. A 1,3-dipole compound comprises a three-atom π-electron system containing four electrons delocalized over the three atoms; 1,3-dipole compounds, i.e. a compound comprising a 1,3-dipole functional group, are well-known in the art. Also, a person skilled in the art knows to select suitable reaction conditions for carrying out the cycloaddition reactions, e.g. hetero-Diels-Alder reaction or 1,3-dipolar cycloaddition. For example, the cycloaddition reaction may be performed for a suitable reaction time in a suitable solvent, for example, dichloromethane, chloroform, tetrahydrofuran (THF), Me-THF, ethyl acetate, diethyl ether, DMF, DMA, DMSO, toluene, benzene, xylene, acetone or hexane; the cycloaddition can be also performed in water, or a mixture of water and a water miscible solvent (e.g. acetonitrile or THF), also suitable buffer systems can be used when performing the reaction with a biomolecule. For example, the reaction may be carried out in DMF, DMSO, a DMF/water mixture, or a DMSO/water mixture. In particular, the reaction may be carried out in DMF, DMSO, a DMF/water mixture, or a DMSO/water mixture when a biomolecule, such as e.g. a protein, an antibody, a peptide, a nucleotide or an oligonucleotide, is reacted. The solvent may be also an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), or borate-containing phosphate-buffered saline. Carrying out the reaction in a buffer is preferred in case a biomolecule, such as e.g. a protein, an antibody, a peptide, a nucleotide or an oligonucleotide, is employed in the reaction. The reaction may be also carried out in a mixture of any one of the aforementioned aqueous buffers and DMF, or DMSO. Suitable solvents and buffers will be readily selected by a person skilled in the art. Alternatively, the reaction can be performed without any solvent (neat). Optionally, the cycloaddition can be performed in the presence of a suitable catalyst. In preferred embodiments, the cycloaddition is a 1,3-dipolar cycloaddition. Suitable 1,3-dipole compounds and reaction conditions are described, e.g., in US 2017/0008858, the entire content of which is hereby incorporated by reference. In particular, azides (e.g., ●-N₃) nitrones (e.g.,

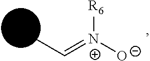

wherein R₆ is as defined herein), nitrile oxides (e.g.,

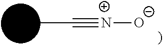

or diazo compounds (e.g.,

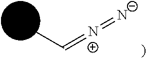

may be used as the 1,3-dipole compound.

Preferably, the 1,3-dipole compound is an azide. The products with an azide, as an exemplary reagent, are shown in the following scheme; as readily appreciated by a person skilled in the art, in principle two regioisomers can be formed (in some embodiments, [symbol] is a triple bond, V is absent, X is R₃C, preferably R₃, R₅ and Rₓ are the same, more preferably, R₃, R₅ and Rₓ are each hydrogen):

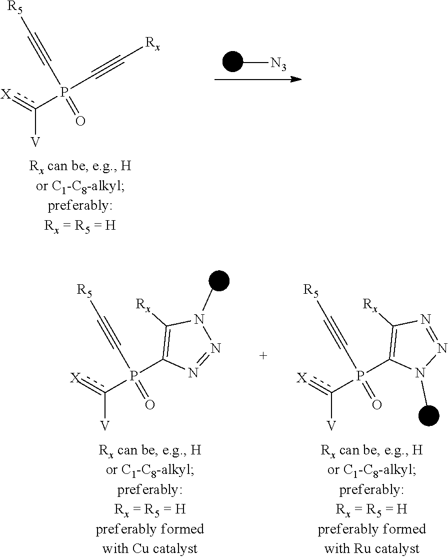

Preferably, regioselectivity in the 1,3-dipolar cycloaddition with an azide can be achieved by using a suitable catalysts. For example, as known to a person skilled in the art, a copper-catalyzed azide/alkyne click reaction using a copper catalyst, preferably a copper(I) catalyst, results in the product shown on the left in the above scheme; preferably, copper(I) bromide is used as catalyst. On the other hand, the product shown in the above scheme on the right is formed when a ruthenium catalyst is used, preferably a ruthenium(II) catalyst, more preferably Cp*RuCl(PPh₃)₂, Cp*RuCl(COD), or Cp*RuCl(NBD) (e.g., B. C. Boren et al., Ruthenium-Catalyzed Azide-Alkyne Cycloaddition: Scope and Mechanism, J. Am. Chem. Soc. 2008, 130, 28, 8923-8930, https://doi.org/10.1021/ja0749993). A person skilled in the art knows to readily select a suitable catalyst and suitable reaction conditions. Other exemplary 5-membered heterocyclic moieties obtained by 1,3-cycloaddition are

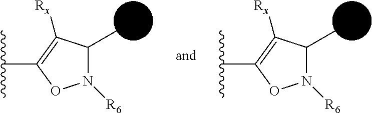

(obtained from a nitrone, R₆ is as defined herein)

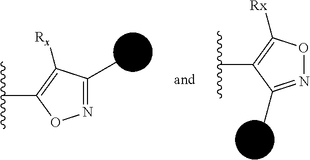

(obtained from a nitrile oxide),

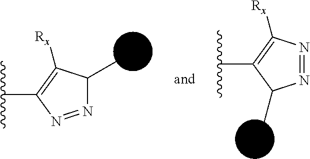

(obtained from a diazo compound), and

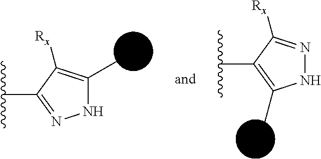

(rearrangement product obtained from a diazo compound).

Compounds of formula (IV) wherein Y is a bond, or compounds of formula (IV*) wherein ▴ is —Y—R₁ and Y is a bond, or preferably compounds of formula (IV*) wherein ▴ is Z and Z represents a residue bound to the phosphorus via a carbon atom and comprising a group ●, wherein ● represents an optionally substituted aliphatic or optionally substituted aromatic residue, can be also prepared from an optionally substituted phosphine oxide, as shown in the following scheme; in particular when Z is

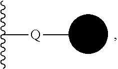

wherein [symbol] indicates the attachment point to the phosphorus and Q is a moiety comprising a carbon carbon triple bond bound to the phosphorus and an optionally substituted phenyl group bound to the carbon-carbon triple bond, or an optionally substituted carbon-carbon double bond bound to the carbon-carbon triple bond (Rₓ is H; in some embodiments, [symbol] is a triple bond, V is absent, X is R₃C, preferably R₃ and R₅ are the same, more preferably, R₃ and R₅ are each hydrogen so that R₃, R₅ and Rₓ are each hydrogen; OTf=triflate):

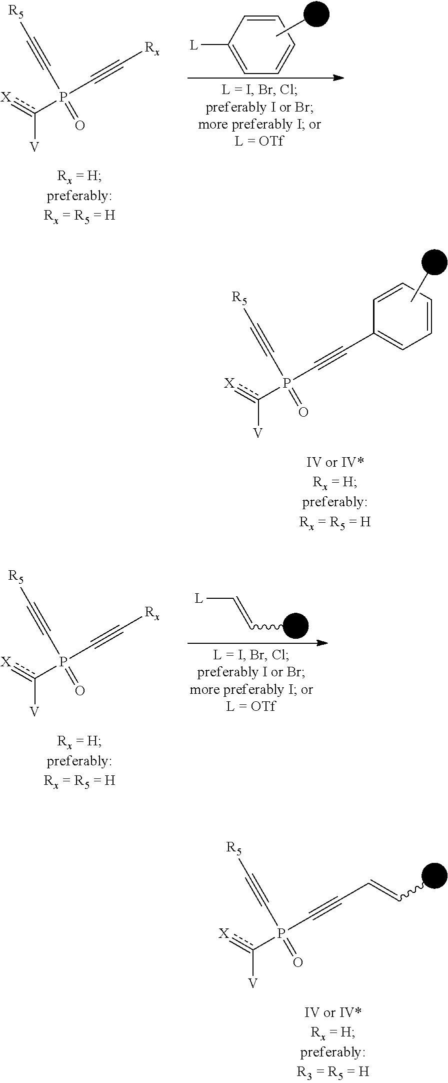

Coupling reactions as depicted in the above scheme and suitable conditions therefore are known to a person skilled in the art, e.g. Cacchi coupling, Castro-Stevens coupling, and Sonogashira coupling. As a non-limiting example, the coupling reaction can be carried out as Sonogashira coupling, using a palladium catalyst and a copper catalyst in the presence of a base. Typically, two catalysts are used for the Sonogashira coupling: a zerovalent palladium complex and a copper(I) halide salt. Common examples of palladium catalysts include those containing phosphine ligands such as [Pd(PPh₃)₄]. Another commonly used palladium source is [Pd(PPh₃)₂Cl₂], but complexes containing bidentate phosphine ligands, such as [Pd(dppe)Cl₂], [Pd(dppp)Cl₂], and [Pd(dppf)Cl₂] can also be used. Copper(I) salts, such as CuI, react with the terminal alkyne and produce a copper(I) acetylide, which acts as an activated species for the coupling reactions. Cu(I) is a co-catalyst in the reaction, and is used to increase the rate of the reaction. A person skilled in the art knows to select suitable conditions for carrying out a Sonogashira coupling. For example, the Sonogashira coupling is carried out at room temperature with a base, typically an amine, such as diethylamine, that may also act as the solvent, but also DMF, DMSO, or ether can be used as solvent. Other bases such as potassium carbonate or cesium carbonate can be used. For example, the reaction may be carried out in DMF, DMSO, a DMF/water mixture, or a DMSO/water mixture. In particular, the reaction may be carried out in DMF, DMSO, a DMF/water mixture, or a DMSO/water mixture when a biomolecule, such as e.g. a protein, an antibody, a peptide, a nucleotide or an oligonucleotide, is reacted. The solvent may be also an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), or borate-containing phosphate-buffered saline. Carrying out the reaction in a buffer is preferred in case a biomolecule, such as e.g. a protein, an antibody, a peptide, a nucleotide or an oligonucleotide, is employed in the reaction. The reaction may be also carried out in a mixture of any one of the aforementioned aqueous buffers and DMF, or DMSO. Suitable solvents and buffers will be readily selected by a person skilled in the art. Preferably, the Sonogashira reaction is carried out under an inert gas, such as e.g. argon.

Still further, compounds of formula (IV) wherein Y is a bond, or compounds of formula (IV*) wherein ▴ is —Y—R₁ and Y is a bond, can be prepared from aryl-substituted dihalophosphine oxide (i.e. R₁ may be aryl or heteroaryl, which may be optionally substituted; preferably R₁ may be phenyl, which may be optionally substituted), e.g. from commercially available dichloro-phenylphosphine oxide, via Grignard reaction, as shown in the following scheme:

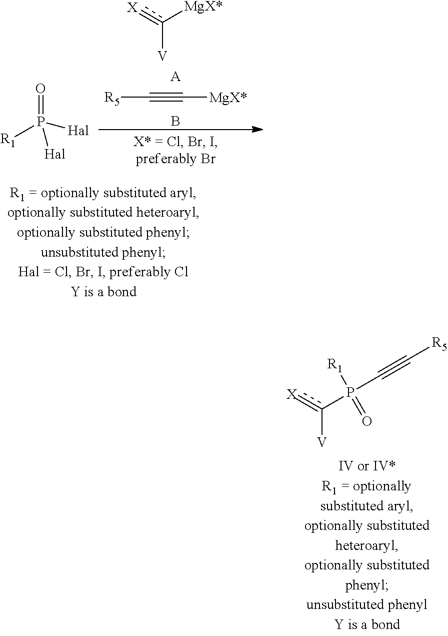

Grignard reaction can be effected under various conditions described herein, e.g. at −78° C. to room temperature, and in a suitable solvent, e.g. tetrahydrofuran or diethyl ether; A and B can be same or different and, where appropriate, can be added in sequential order; preferably, A and B are the same, in A the [symbol] is a triple bond, V is absent, X is R₃C, R₃ and R₅ are the same; more preferably R₃ and R₅ are hydrogen. Also, work-up and optional purification can be carried out as described herein.

Compounds of formula (IV) or (IV*), wherein [symbol] is a double bond and X is R₃R₄C, preferably, R₃, R₄ and V are each hydrogen, can be, as illustrative example, prepared as follows:

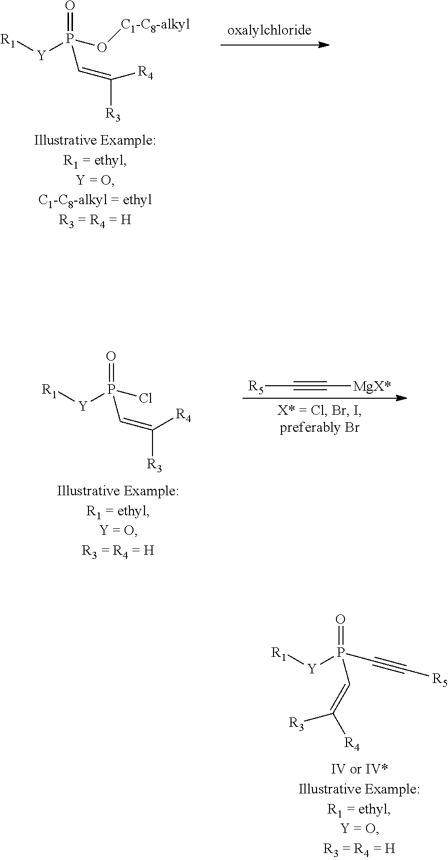

Accordingly, for example, activation can be achieved using oxalylchloride to form a chlorinated intermediate. The chlorination can be carried out, e.g., at temperatures between 0° C. and 60° C., for example at about 30° C. A person skilled in the art knows to select a suitable solvent; e.g., dichloromethane can be used. The chlorinated intermediate can be isolated, e.g., via evaporation of the solvent or, alternatively, used in the following reaction step without prior isolation. Preferably, when using dichloromethane as solvent for the chlorination, the solvent is removed, before the Grignard reaction is carried out. The Grignard reaction can be carried out as described in numerous instances herein, e.g. using diethyl ether or tetrahydrofuran as solvent, e.g. at a temperature at from −78° C. to room temperature. The reaction mixture can be subjected to work-up using procedures known to a person skilled in the art, and can be optionally purified, if desired, using e.g. chromatography.

As provided herein by describing the numerous synthetic procedures, a person skilled in the art is able to synthesize the compounds of formula (IV) or formula (IV*) as described throughout this specification and the appended claims, and will readily select suitable starting materials, reagents and reaction conditions.

### Synthesis of Compounds of Formula (I)

Synthetic methods to provide compounds of formula (I), wherein an aliphatic or aromatic residue Z is bound to the phosphorus atom via a carbon atom, are generally known to a person skilled in the art, and some general features are described in an exemplary manner in the following. For example, as readily appreciated by a person skilled in the art, Grignard reactions are suitable for forming a carbon-phosphorus bond. Numerous further methods, e.g. as those described in the following, can be used. In general, a person skilled in the art knows to select suitable starting materials and reaction conditions for carrying out such syntheses. Any variables, e.g. R₁, R², R₃, R₄, R₅, V, X, Y, Z, [symbol] , [symbol] , ●, [symbol] and any others, are as defined throughout this specification, if not noted otherwise.

As an illustrative example, compounds of formula (I) can be prepared from compounds of formula (IV), e.g., according to the following scheme, in particular when Z is

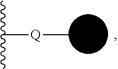

wherein [symbol] indicates the attachment point to the phosphorus and Q is a moiety comprising at least three main-chain atoms and a carbon-carbon double bond, wherein at least one of the main chain atoms is a heteroatom selected from the group consisting of S, O or N:

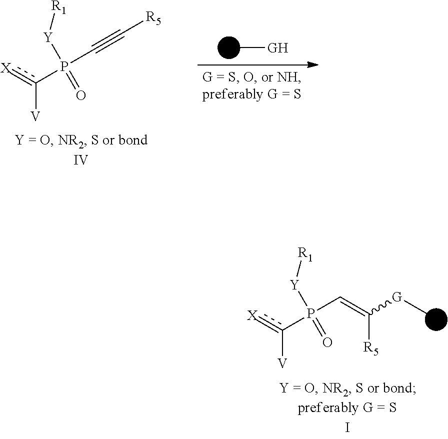

In these embodiments the compound of formula (I) is formed by addition of ●-GH, wherein G is S, O, or NH, to the triple bond of a compound of formula (IV) to form a compound of formula (I); preferably, G is S. In some embodiments, [symbol] is a triple bond, V is absent, X is R₃C, preferably R₃, R₅ and Rₓ are the same, more preferably, R₃, R₅ and Rₓ are each hydrogen. The reaction can be carried out, e.g., as described above for the synthesis of compounds of formula (IV) or (IV*). Thus, the reaction is carried out in a suitable solvent, which is readily determined by a person skilled in the art. The solvent system can be chosen from a wide range of solvents. The solvent can be a polar aprotic solvent system such as tetrahydrofuran (THF), dimethylformamide (DMF), acetonitrile (MeCN), acetone, dimethyl sulfoxide (DMSO), ethyl acetate (EtOAc), N-ethylpyrrolidone or mixtures thereof, preferably THF, DMF, DMSO; nonpolar solvents such as hexane, toluene, benzene, 1,4-dioxane, chloroform, diethyl ether or dichloromethane (DCM), preferably DCM; polar protic solvents such as water, ethanol, isopropanol, methanol, n-butanol, preferably ethanol; or mixtures thereof. For example, the reaction may be carried out in DMF, DMSO, a DMF/water mixture, or a DMSO/water mixture. In particular, the reaction may be carried out in DMF, DMSO, a DMF/water mixture, or a DMSO/water mixture when a biomolecule, such as e.g. a protein, an antibody, a peptide, a nucleotide or an oligonucleotide, is reacted. The solvent may be also an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), or borate-containing phosphate-buffered saline. Carrying out the reaction in a buffer is preferred in case a biomolecule, such as e.g. a protein, an antibody, a peptide, a nucleotide or an oligonucleotide, is employed in the reaction. The reaction may be also carried out in a mixture of any one of the aforementioned aqueous buffers and DMF, or DMSO. Suitable solvents and buffers will be readily selected by a person skilled in the art. Preferably, the reaction is carried out under basic conditions, in particular under slightly basic conditions, e.g. at a pH of e.g. between 7.2 and 9, such as e.g. at a pH of 8 or 8.5. Such basic conditions may be established by using a suitable buffer system, such as e.g. by using any one of the buffers mentioned above. In addition or alternatively, basic conditions for the reaction may be established by using a weak base. Suitable bases are e.g. carbonates such as (NH₄)₂CO₃, Na₂CO₃, Rb₂CO₃, K₂CO₃ or Cs₂CO₃ or correlating hydrogencarbonates thereof (e.g. NaHCO₃ etc.); and weak nitrogen-containing bases such as trimethylamine Et₃N (pKa 10,76 at 25° C.). Preferably, a base with a pKₐ value within the range of 7,5 to 11,5 is used. Suitable bases will be readily selected by a person skilled in the art. The reaction temperature is not particularly limited. For example, the reaction may be carried out at temperatures in a range of from 0° C. to 60° C., of from 0° C. to 50° C., of from 0° C. to 40° C., of from 0° C. to 30° C., e.g. at room temperature, i.e. around 25° C., e.g. at around 5° C., or e.g. at physiologically relevant conditions at around 37° C. Suitable reaction conditions, including temperatures and reaction times, will be readily selected by a person skilled in the art.

Compounds of formula (I) can be also prepared from compounds of formula (IV)

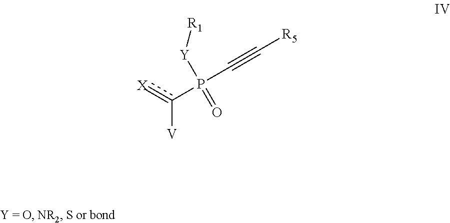

using, e.g., cycloaddition reactions, in particular when Z is

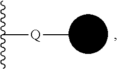

wherein [symbol] indicates the attachment point to the phosphorus, and Q is a five- or six-membered heterocyclic moiety comprising 1, 2 or 3 heteroatoms independently selected from the group consisting of N, O or S. Such heterocyclic moiety can be formed, e.g., using cycloaddition reactions, as known to a person skilled in the art. For example, a six-membered heterocyclic moiety may be obtained by reacting the compound of formula (IV) with a suitable hetero-diene in a hetero-Diels-Alder reaction. A five-membered heterocyclic moiety may be formed by reacting the compound of formula (IV) with a suitable 1,3-dipole compound in a 1,3-dipolar cycloaddition (the 1,3-dipolar cycloaddition is also known as click reaction). Suitable hetero-dienes and 1,3-dipole compounds are known to a person skilled in the art and readily selected. A 1,3-dipole compound comprises a three-atom π-electron system containing four electrons delocalized over the three atoms; 1,3-dipole compounds, i.e. a compound comprising a 1,3-dipole functional group, are well-known in the art. Also, a person skilled in the art knows to select suitable reaction conditions for carrying out the cycloaddition reactions, e.g. hetero-Diels-Alder reaction or 1,3-dipolar cycloaddition. For example, the cycloaddition reaction may be performed for a suitable reaction time in a suitable solvent, for example, dichloromethane, chloroform, tetrahydrofuran (THF), Me-THF, ethyl acetate, diethyl ether, DMF, DMA, DMSO, toluene, benzene, xylene, acetone or hexane; the cycloaddition can be also performed in water, or a mixture of water and a water miscible solvent (e.g. acetonitrile or THF), also suitable buffer systems can be used when performing the reaction with a biomolecule. For example, the reaction may be carried out in DMF, DMSO, a DMF/water mixture, or a DMSO/water mixture. In particular, the reaction may be carried out in DMF, DMSO, a DMF/water mixture, or a DMSO/water mixture when a biomolecule, such as e.g. a protein, an antibody, a peptide, a nucleotide or an oligonucleotide, is reacted. The solvent may be also an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), or borate-containing phosphate-buffered saline. Carrying out the reaction in a buffer is preferred in case a biomolecule, such as e.g. a protein, an antibody, a peptide, a nucleotide or an oligonucleotide, is employed in the reaction. The reaction may be also carried out in a mixture of any one of the aforementioned aqueous buffers and DMF, or DMSO. Suitable solvents and buffers will be readily selected by a person skilled in the art. Alternatively, the reaction can be performed without any solvent (neat). Optionally, the cycloaddition can be performed in the presence of a suitable catalyst. In preferred embodiments, the cycloaddition is a 1,3-dipolar cycloaddition. Suitable 1,3-dipole compounds and reaction conditions are described, e.g., in US 2017/0008858, the entire content of which is hereby incorporated by reference. In particular, azides (e.g., ●-N3) nitrones (e.g.,

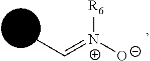

wherein R₆ is as defined herein), nitrile oxides (e.g.,

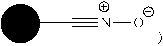

or diazo compounds (e.g.,

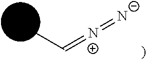

may be used as the 1,3-dipole compound. Preferably, the 1,3-dipole compound is an azide. The products with an azide, as an exemplary reagent, are shown in the following scheme; as readily appreciated by a person skilled in the art, in principle two regioisomers can be formed (in some embodiments, [symbol] is a triple bond, V is absent, X is R₃C, preferably R₃ and R₅ are the same, more preferably, R₃ and R₅ are each hydrogen; in some embodiments, [symbol] is a double bond, X is R₃R₄C, preferably V, R₃, R₄ and R₅ are each H):

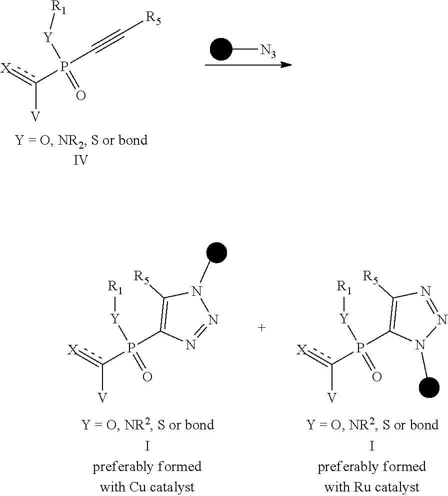

Preferably, regioselectivity in the 1,3-dipolar cycloaddition with an azide can be achieved by using a suitable catalysts. For example, as known to a person skilled in the art, a copper-catalyzed azide/alkyne click reaction using a copper catalyst, preferably a copper(I) catalyst, results in the product shown on the left in the above scheme; preferably, copper(I) bromide is used as catalyst. On the other hand, the product shown on the right in the above scheme is formed when a ruthenium catalyst is used, preferably a ruthenium(II) catalyst, more preferably Cp*RuCl(PPh₃)₂, Cp*RuCl(COD), or Cp*RuCl(NBD) (e.g., B. C. Boren et al., Ruthenium-Catalyzed Azide-Alkyne Cycloaddition: Scope and Mechanism, J. Am. Chem. Soc. 2008, 130, 28, 8923-8930, https://doi.org/10.1021/ja0749993). A person skilled in the art knows to readily select a suitable catalyst and suitable reaction conditions. Other exemplary 5-membered heterocyclic moieties obtained by 1,3-cycloaddition are

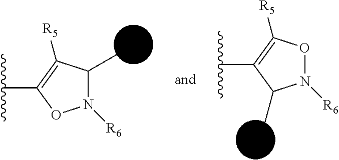

(obtained from a nitrone, R₆ is as defined herein)

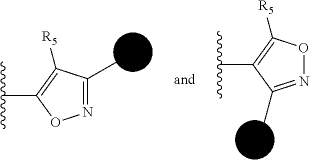

(obtained from a nitrile oxide),

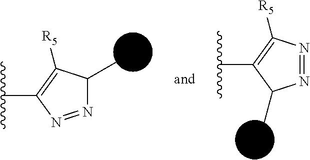

(obtained from a diazo compound), and

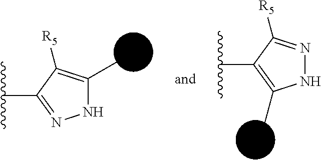

(rearrangement product obtained from a diazo compound).

Compounds of formula (I) can be also prepared, e.g., as indicated in the following scheme, using C—C coupling reactions, in particular when Z is

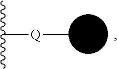

wherein [symbol] indicates the attachment point to the phosphorus and Q is a moiety comprising a carbon carbon triple bond bound to the phosphorus and an optionally substituted phenyl group bound to the carbon-carbon triple bond, or an optionally substituted carbon-carbon double bond bound to the carbon-carbon triple bond (R₅ is H; in some embodiments, [symbol] is a triple bond, V is absent, X is R₃C, preferably R₃ and R₅ are the same, more preferably, R₃ and R₅ are each hydrogen; OTf=triflate):

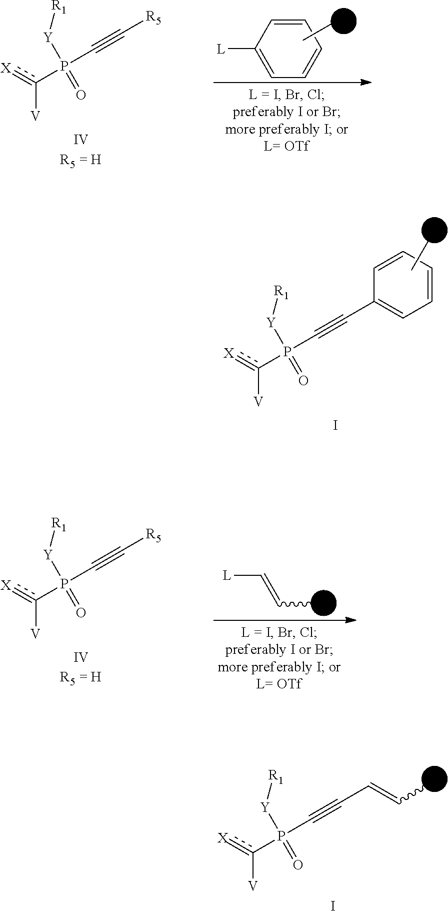

Coupling reactions as depicted in the above scheme and suitable conditions therefore are known to a person skilled in the art, e.g. Cacchi coupling, Castro-Stevens coupling, and Sonogashira coupling. As a non-limiting example, the coupling reaction can be carried out as Sonogashira coupling, using a palladium catalyst and a copper catalyst in the presence of a base. Typically, two catalysts are used for the Sonogashira coupling: a zerovalent palladium complex and a copper(I) halide salt. Common examples of palladium catalysts include those containing phosphine ligands such as [Pd(PPh₃)₄]. Another commonly used palladium source is [Pd(PPh₃)₂Cl₂], but complexes containing bidentate phosphine ligands, such as [Pd(dppe)Cl₂], [Pd(dppp)Cl₂], and [Pd(dppf)Cl₂] can also be used. Copper(I) salts, such as CuI, react with the terminal alkyne and produce a copper(I) acetylide, which acts as an activated species for the coupling reactions. Cu(I) is a co-catalyst in the reaction, and is used to increase the rate of the reaction. A person skilled in the art knows to select suitable conditions for carrying out a Sonogashira coupling. For example, the Sonogashira coupling is carried out at room temperature with a base, typically an amine, such as diethylamine, that may also act as the solvent, but also DMF, DMSO, or ether can be used as solvent. Other bases such as potassium carbonate or cesium carbonate can be used. For example, the reaction may be carried out in DMF, DMSO, a DMF/water mixture, or a DMSO/water mixture. In particular, the reaction may be carried out in DMF, DMSO, a DMF/water mixture, or a DMSO/water mixture when a biomolecule, such as e.g. a protein, an antibody, a peptide, a nucleotide or an oligonucleotide, is reacted. The solvent may be also an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), or borate-containing phosphate-buffered saline. Carrying out the reaction in a buffer is preferred in case a biomolecule, such as e.g. a protein, an antibody, a peptide, a nucleotide or an oligonucleotide, is employed in the reaction. The reaction may be also carried out in a mixture of any one of the aforementioned aqueous buffers and DMF, or DMSO. Suitable solvents and buffers will be readily selected by a person skilled in the art. Preferably, the Sonogashira reaction is carried out under an inert gas, such as e.g. argon.

### Synthesis of Compounds of Formula (III)

Compounds of formula (I) can be subjected to hydrothiolation reaction with a thiol of formula (II), as shown in the following scheme:

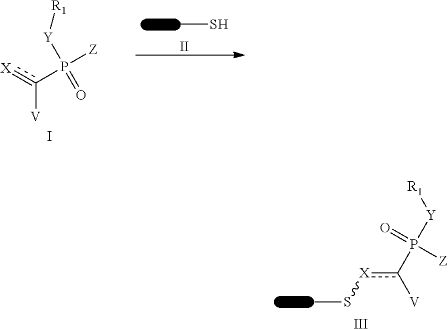

Any variables, e.g. R₁, V, X, Y, Z, [symbol] , [symbol] , ●, [symbol] and any others, are as defined throughout this specification, if not noted otherwise. The reaction can be carried out in a suitable solvent. The solvent system can be chosen from a wide range of solvents. The solvent can be a polar aprotic solvent system such as tetrahydrofuran (THF), dimethylformamide (DMF), acetonitrile (MeCN), acetone, dimethyl sulfoxide (DMSO), ethyl acetate (EtOAc), N-ethylpyrrolidone or mixtures thereof, preferably THF, DMF, DMSO; nonpolar solvents such as hexane, toluene, benzene, 1,4-dioxane, chloroform, diethyl ether or dichloromethane (DCM), preferably DCM; polar protic solvents such as water, ethanol, isopropanol, methanol, n-butanol, preferably ethanol; or mixtures thereof. For example, the reaction may be carried out in DMF, DMSO, a DMF/water mixture, or a DMSO/water mixture. In particular, the reaction may be carried out in DMF, DMSO, a DMF/water mixture, or a DMSO/water mixture when a biomolecule, such as e.g. a protein, an antibody, a peptide, a nucleotide or an oligonucleotide, is reacted. The solvent may be also an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), or borate-containing phosphate-buffered saline. Carrying out the reaction in a buffer is preferred in case a biomolecule, such as e.g. a protein, an antibody, a peptide, a nucleotide or an oligonucleotide, is employed in the reaction. The reaction may be also carried out in a mixture of any one of the aforementioned aqueous buffers and DMF, or DMSO. Suitable solvents and buffers will be readily selected by a person skilled in the art. Preferably, the hydrothiolation reaction of a phosphonothiolate or a phosphonate is carried out under basic conditions, in particular under slightly basic conditions, e.g. at a pH of e.g. between 7.2 and 9, such as e.g. at a pH of 8 or 8.5. Such basic conditions may be established by using a suitable buffer system, such as e.g. by using any one of the buffers mentioned above. In addition or alternatively, basic conditions for the hydrothiolation reaction may be established by using a weak base. Suitable bases are e.g. carbonates such as (NH₄)₂CO₃, Na₂CO₃, Rb₂CO₃, K₂CO₃ or Cs₂CO₃ or correlating hydrogencarbonates thereof (e.g. NaHCO₃ etc.); and weak nitrogen-containing bases such as trimethylamine Et₃N (pKa 10,76 at 25° C.). Preferably, a base with a pKₐ value within the range of 7,5 to 11,5 is used. Suitable bases will be readily selected by a person skilled in the art. The reaction temperature of the hydrothiolation is not particularly limited. For example, the hydrothiolation may be carried out at temperatures in a range of from 0° C. to 60° C., of from 0° C. to 50° C., of from 0° C. to 40° C., of from 0° C. to 30° C., e.g. at room temperature, i.e. around 25° C., e.g. at around 5° C., or e.g. at physiologically relevant conditions at around 37° C. The reaction time depends on the temperature, the reaction volume and the amount of substance. As a guideline, the reaction could be e.g. carried out in a time frame from 1 minute to 24 hours, e.g. in a time frame of from 1 minute to 20 hours, of from 1 minute to 10 hours, of from 1 minute to 3 hours, or even within a time frame between 1 minute and 1 hour. Suitable reaction temperatures and reaction times will be readily determined by a person skilled in the art. Similarly, suitable conditions for work-up of the reaction mixture and purification, e.g. including chromatography, are readily determined by the skilled person.

### Synthesis of a Conjugate of an Antibody Molecule

The preparation of a conjugate of an antibody molecule in particular includes reducing a disulfide bridge of an antibody molecule and reacting said antibody molecule with a compound of formula (IV*), as exemplarily shown in the following scheme:

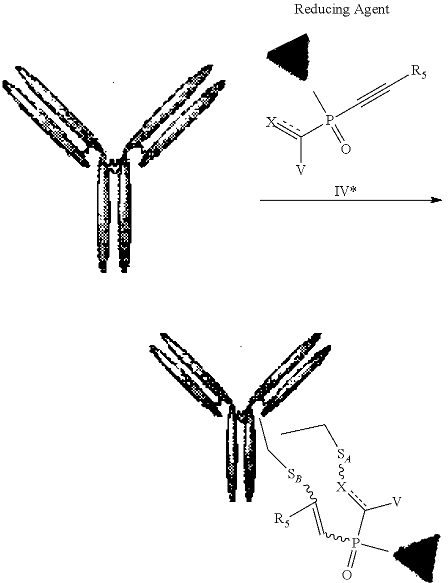

Accordingly, a conjugate of an antibody molecule is obtained which comprises a moiety of formula (V):

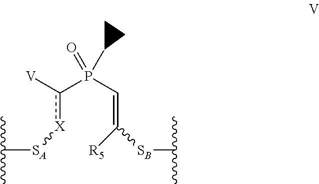

Any variables, e.g. R₅, V, X, [symbol] , [symbol] , ▴, S_{A}, S_{B} and any others, are as defined throughout this specification, if not noted otherwise. Suitable reducing agents for the reduction of a disulfide bridge are known to a person skilled in the art and can be readily selected. For example, among others, tris(2-carboxyethyl)phosphine (TCEP), dithiothreitol (DTT), sodium dithionite, sodium thiosulfate, or sodium sulfite can be used; for example, the reducing agent may be tris(2-carboxyethyl)phoshine (TCEP). Suitable reaction conditions, including concentration, temperature and time, are readily selected by the skilled person. Also, suitable conditions for reaction of the sulfhydryl groups with the compound of formula (IV*) are readily determined by a person skilled in the art and may include conditions as already described herein for the synthesis of compound of formula (III) from a compound of formula (I) with a compound of formula (11). For example, the reduction and reaction with a compound of formula (IV*) can be carried out at of from 0° C. to 50° C., of from 0° C. to 40° C., of from 0° C. to 30° C., e.g. at room temperature, i.e. around 25° C., e.g. at around 5° C., or e.g. at physiologically relevant conditions at around 37° C.; the reaction may be carried out in a time frame from 1 minute to 24 hours, e.g. overnight. As an illustrative example, reduction of a disulfide bridge can be achieved within 30 min using 10 eq. TCEP at 37° C. and pH 8.4; and then subsequently 5 eq. of the compound of formula (IV)* can be added, and the reaction is carried out overnight. The solvent can be any solvent or buffer system typically used for reactions of a biomolecule, in particular of an antibody, and suitable solvents and buffer systems may include those used for the reaction of a compound of formula (I) with a compound of formula (II) to give a compound of formula (III), as described herein. In particular, the reaction may be carried out in an aqueous medium, such as e.g. water or an aqueous buffer, such as e.g. phosphate-buffered saline (PBS), tris(hydroxymethyl)-aminomethane (TRIS), bicarbonate, EDTA/NH₄HCO₃ buffer, EDTA/NH₄HCO₃ in phosphate buffered saline (PBS), borate-containing phosphate-buffered saline, or TRIS/NaCl/EDTA. The reaction may be also carried out in a mixture of any one of the aforementioned aqueous buffers and DMF, or DMSO. Suitable solvents and buffers will be readily selected by a person skilled in the art. Preferably, the reaction with a compound of formula (IV*) is carried out under basic conditions, in particular under slightly basic conditions, e.g. at a pH of e.g. between 7.2 and 9, such as e.g. at a pH of 8 or 8.5. Such basic conditions may be established by using a suitable buffer system, such as e.g. by using any one of the buffers mentioned above. In addition or alternatively, basic conditions may be established by using a weak base. Suitable bases are e.g. carbonates such as (NH₄)₂CO₃, Na₂CO₃, Rb₂CO₃, K₂CO₃ or Cs₂CO₃ or correlating hydrogencarbonates thereof (e.g. NaHCO₃ etc.); and weak nitrogen-containing bases such as trimethylamine Et₃N (pKa 10,76 at 25° C.). Preferably, a base with a pKₐ value within the range of 7,5 to 11,5 is used. Suitable bases will be readily selected by a person skilled in the art. As illustrative examples, the reaction can be carried out in TRIS/NaCl/EDTA at a pH of 8.4, e.g. at 50 mM TRIS, 1 mM EDTA and 300 mM NaCl at a pH of 8.4. Further, Suitable conditions for work-up of the reaction mixture and/or purification, e.g. including buffer exchange using a spin desalting column, are readily determined by a skilled person.

### Items of the Invention

### The Invention Further Relates to the Following Items:

1. A method of preparing a compound of formula (III) comprising a step of:
reacting a compound of formula (I)

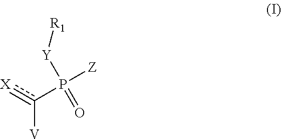

wherein
[symbol] represents a triple bond or a double bond;
V is absent when [symbol] is a triple bond; or
V represents H or C₁-C₈-alkyl when [symbol] is a double bond;
X represents R₃—C when [symbol] is a triple bond; or
X represents

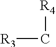

when [symbol] is a double bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl; and
Z represents a residue bound to the phosphorus via a carbon atom and comprising a group ●, wherein ● represents an optionally substituted aliphatic or optionally substituted aromatic residue;
with a thiol-containing molecule of formula (II)

[symbol]

-13 SH (II)
wherein [symbol] represents an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system;
resulting in a compound of formula (III)

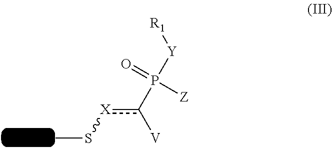

wherein
[symbol] represents a double bond when [symbol] in a compound of formula (I) represents a triple bond; or
[symbol] represents a bond when [symbol] in a compound of formula (I) represents a double bond;
V is absent when [symbol] is a double bond; or
V represents H or C₁-C₈-alkyl when [symbol] is a bond;
X represents R₃—C when [symbol] is a double bond; or
X represents

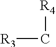

when [symbol] is a bond; and
[symbol] , R₁, R₃, R₄, Y and Z are as defined above.

2. The method according to item 1, wherein [symbol] represents a triple bond; V is absent; X represents R₃—C; R₃ represents H or C₁-C₈-alkyl, preferably R₃ is H, and [symbol] represents a double bond.

3. The method according to item 1, wherein [symbol] represents a double bond; V represents H or C₁-C₈-alkyl, preferably V is H; X represents

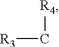

R₃ represents H or C₁-C₈-alkyl, preferably R₃ is H; R₄ represents H or C₁-C₈-alkyl, preferably R₄ is H; and [symbol] represents a bond.

4. The method according to any one of the preceding items, wherein Z is

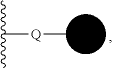

wherein [symbol] indicates the attachment point to the phosphorus and ● is as defined in any one of the preceding items; and
Q is a moiety comprising at least three main-chain atoms and a carbon-carbon double bond, wherein at least one of the main chain atoms is selected from the group consisting of S, O or N; wherein optionally a linker is arranged between ● and Q.

5. The method according to item 4, wherein Z is

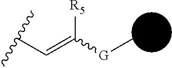

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items; wherein optionally a linker is arranged between ● and Q.

6. The method according to item 5, wherein Z is

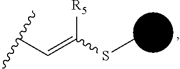

R₅ is H or C₁-C₈-alkyl, preferably R₅ is H, and ● is as defined in any one of the preceding items; wherein optionally a linker is arranged between ● and Q.

7. The method according to any one of items 4 to 6, further comprising a preparation of a compound of formula (I), said preparation comprising:
reacting a compound of formula (IV)

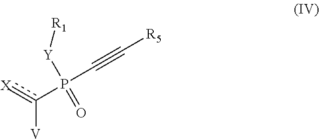

wherein R₁, R₅, [symbol] , V, X and Y are as defined in any one of the preceding items, with ●-GH to form a compound of formula (I), wherein G and ● are as defined in any one of the preceding items; preferably G is S; wherein optionally a linker is arranged between ● and Q.

8. The method according to any one of items 1 to 3, wherein Z is

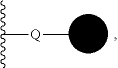

wherein [symbol] indicates the attachment point to the phosphorus and ● is as defined in any one of the preceding items; and
Q is a five- or six-membered heterocyclic moiety comprising 1, 2 or 3 heteroatoms independently selected from the group consisting of N, O or S; wherein optionally a linker is arranged between ● and Q.

9. The method according to item 8, wherein Z is selected from the group consisting of

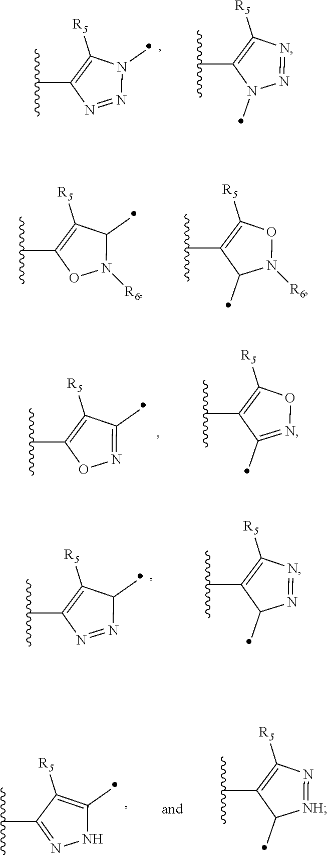

wherein R₅ is H or C₁-C₈-alkyl, preferably R₅ is H; R₆ is C₁-C₈-alkyl, and ● is as defined in any one of the preceding items; wherein optionally a linker is arranged between ● and Q.

10. The method according to item 9, wherein Z is

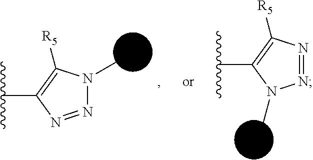

preferably Z is

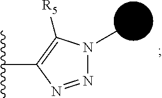

R₅ is H or C₁-C₈-alkyl, preferably R₅ is H; and ● is as defined in any one of the preceding items; wherein optionally a linker is arranged between ● and Q.

11. The method according to any one of items 8 to 10, further comprising a preparation of the compound of formula (I), said preparation comprising:
reacting a compound of formula (IV)

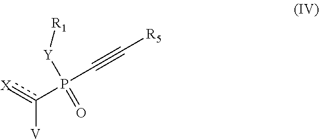

wherein R₁, R₅, [symbol] , V, X and Y are as defined in any one of the preceding items, with

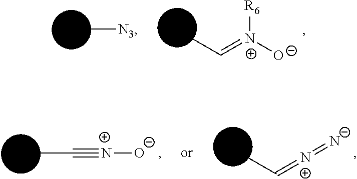

preferably reacting with ●-N₃, to form a compound of formula (I), wherein ● is as defined in any one of the preceding items; R₆ is C₁-C₈-alkyl; preferably the reacting is carried out in the presence of a catalyst, e.g. a copper catalyst or a ruthenium catalyst; wherein optionally a linker is arranged between ● and Q.

12. The method according to any one of items 1 to 3, wherein Z is

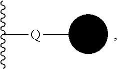

wherein [symbol] indicates the attachment point to the phosphorus and ● is as defined in any one of the preceding items; and
Q is a moiety comprising a carbon-carbon triple bond bound to the phosphorus in the compound of formula (I), and an optionally substituted phenyl group bound to the carbon-carbon triple bond; or
Q is a moiety comprising a carbon-carbon triple bond bound to the phosphorus in formula (I), and an optionally substituted carbon-carbon double bond bound to the carbon-carbon triple bond;
wherein optionally a linker is arranged between ● and Q.

13. The method according to item 12, wherein Z is

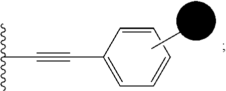

or wherein Z is

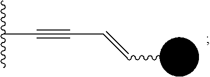

wherein ● is as defined in any one of the preceding items;
wherein optionally a linker is arranged between ● and Q.

14. The method according to item 12 or 13, further comprising a preparation of the compound of formula (I), said preparation comprising:
reacting a compound of formula (IV)

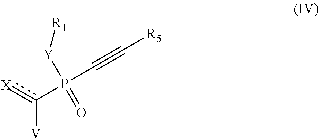

wherein R₁, [symbol] , V, X and Y are as defined in any one of the preceding items and R₅ with

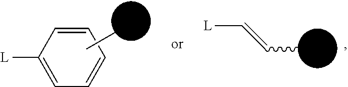

wherein L is halogen (I, Br, Cl, preferably I or Br, more preferably 1) or O-triflate to form a compound of formula (I); preferably, the reacting is carried out in presence of a palladium catalyst, a copper catalyst and a base; wherein optionally a linker is arranged between ● and Q.

15. The method according to any one of the preceding items, wherein Y is O.

16. The method according to any one of items 1 to 14, wherein Y is NR²; preferably wherein R² is C₁-C₈-alkyl, more preferably wherein R² is methyl, ethyl, propyl or butyl, still more preferably wherein R² is methyl or ethyl.

17. The method according to any one of items 1 to 14, wherein Y is S.

18. The method according to any one of items 1 to 14, wherein Y is a bond.

19. The method according to any one of the preceding items, wherein R₁ represents a small molecule; C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; preferably in in each instance Y is O; or R₁ represents phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; preferably in each instance Y is a bond; or R₁ represents a 5- or 6-membered heteroaromatic system such as optionally substituted triazolyl or optionally substituted pyridyl; preferably in each instance Y is a bond.

20. The method according to item 19, wherein R₁ represents a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore.

21. The method according to item 19 or 20, wherein R₁ represents C₁-C₈-alkyl, preferably methyl, ethyl, propyl or butyl, more preferably methyl or ethyl, still more preferably ethyl.

22. The method according to any one of items 1 to 18, wherein R₁ is selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O.

23. The method according to item 22, wherein R₁ is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

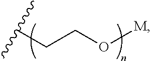

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

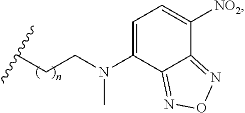

wherein n is 1, 2, 3, 4, 5, 6, 7, 8, 9 or 10, more preferably 4, 5 or 6, still more preferably 5, or more preferably R₁ is

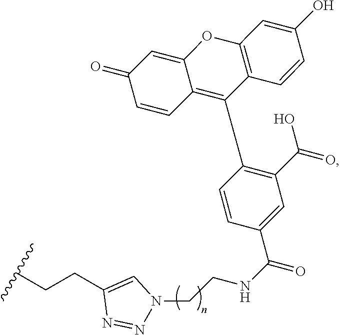

wherein n is 1, 2, 3, 4, 5, 6, 7, 8, 9 or 10, preferably 3, 4 or 5, still more preferably 4; C₂-C₈-alkynyl, preferably

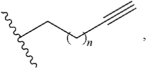

wherein n is 1, 2, 3, 4, or 5, preferably 1, 2 or 3, more preferably 1; or preferably R₁ is

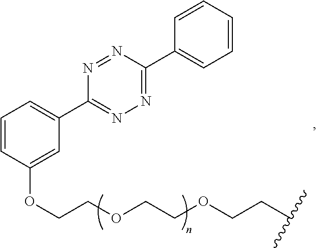

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

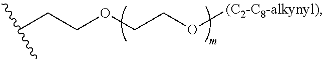

more preferably

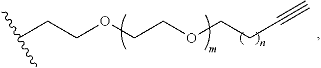

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O.

24. The method according to any one of items 1 to 18, wherein R₁ is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

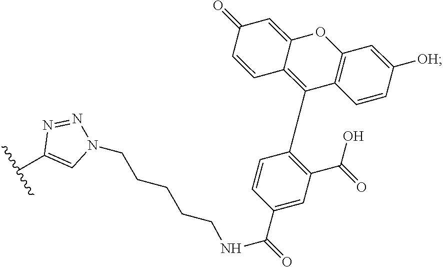 [SMILES: *c1cn(CCCCCNC(=O)c2ccc(-c3c4ccc(=O)cc-4oc4cc(O)ccc34)c(C(=O)O)c2)nn1]

or still more preferably R₁ is

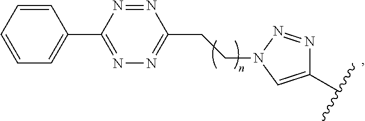

wherein n is 1, 2, 3, 4, 5, 6, 7, 8 or 9, preferably 1, 2 or 3, more preferably 1; or preferably R₁ is

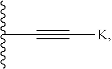

wherein K is H or C₁-C₈-alkyl, preferably K is H; preferably wherein in each instance Y is a bond.

25. The method according to any one of items 1 to 23, wherein R₁ is C₁-C₈-alkyl, preferably methyl, ethyl, propyl or butyl; more preferably methyl or ethyl; still more preferably ethyl; and
Z is

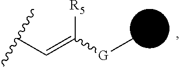

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

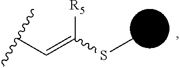

R₅ is H or C₁-C₈-alkyl, preferably R₅ is H, and ● is as defined in any one of the preceding items;
preferably in each instance Y is O;
optionally the C₁-C₈-alkyl is substituted with a fluorophore;
optionally a linker is arranged between ● and Q.

26. The method according to any one of items 1 to 23, wherein R₁ is C₂-C₈-alkynyl, preferably

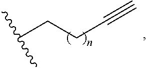

wherein n is 1, 2, 3, 4, or 5, preferably 1, 2 or 3 more preferably 1; or wherein R₁ is

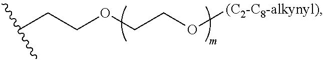

preferably

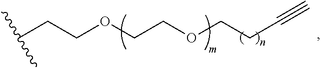

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and
Z is

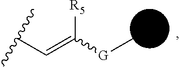

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

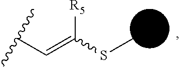

R₅ is H or C₁-C₈-alkyl, preferably R₅ is H, and ● is as defined in any one of the preceding items;
preferably in each instance Y is O;
optionally a linker is arranged between ● and Q.

27. The method according to any one of items 1 to 23, wherein R₁ is C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and
Z is

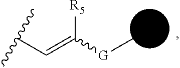

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

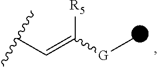

R₅ is H or C₁-C₈-alkyl, preferably R₅ is H, and ● is as defined in any one of the preceding items;
preferably R₁ is

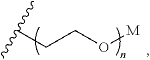

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4;
preferably in each instance Y is O;
optionally a linker is arranged between ● and Q.

28. The method according to any one of items 1 to 23, wherein R₁ is C₁-C₈-alkyl, preferably methyl, ethyl, propyl or butyl; more preferably methyl or ethyl; still more preferably ethyl; and
Z is selected from the group consisting of

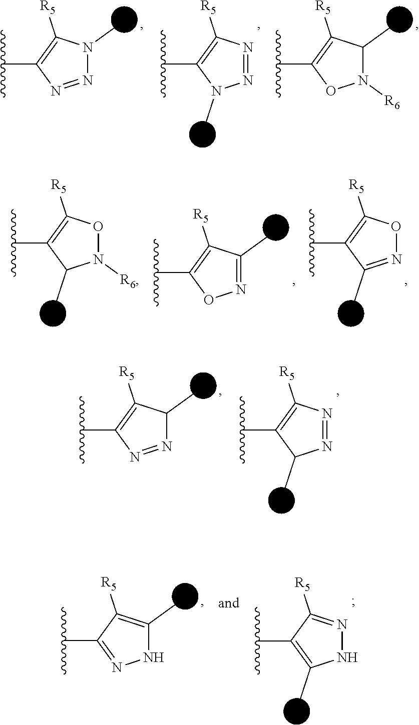

wherein R₅ is H or C₁-C₈-alkyl, preferably R₅ is H; R₆ is C₁-C₈-alkyl; and ● is as defined in any one of the preceding items;
preferably Z is

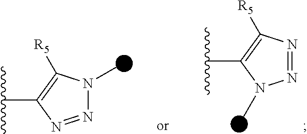

or more preferably Z is

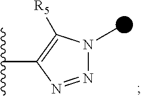

is H or C₁-C₈alkyl, preferably R₅ is H; and ● is as defined in any one of the preceding items;
preferably in each instance Y is O;
optionally the C₁-C₈-alkyl is substituted with a fluorophore;
optionally a linker is arranged between ● and Q.

29. The method according to any one of items 1 to 23, wherein R₁ is C₂-C₈-alkynyl, preferably

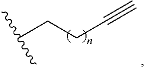

wherein n is 1, 2, 3, 4, 5, 6, 7, 8, 9 or 10, preferably 1, 2 or 3, more preferably 1; or wherein R₁ is

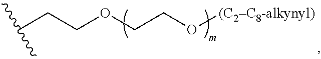

preferably

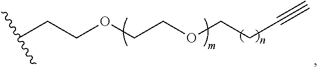

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and
Z is selected from the group consisting of

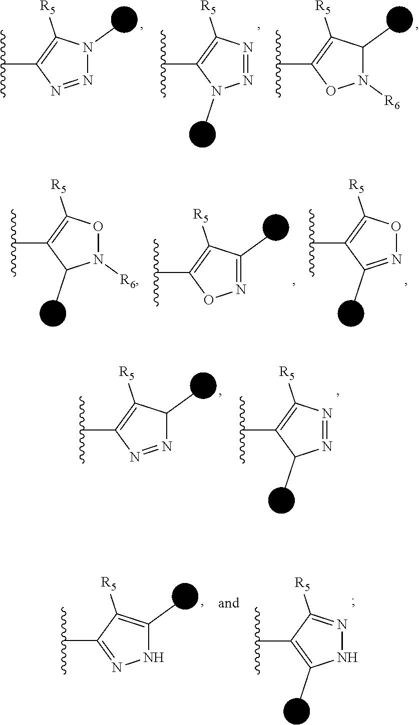

wherein R₅ is H or C₁-C₈-alkyl, preferably R₅ is H; R₆ is C₁-C₈-alkyl; and ● is as defined in any one of the preceding items;
preferably Z is

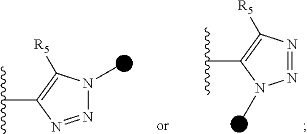

more preferably Z is

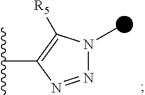

R₅ is H or C₁-C₈-alkyl, preferably R₅ is H; and ● is as defined in any one of the preceding items;
preferably in each instance Y is O;
optionally a linker is arranged between ● and Q.

30. The method according to any one of items 1 to 23, wherein R₁ is C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z is selected from the group consisting of

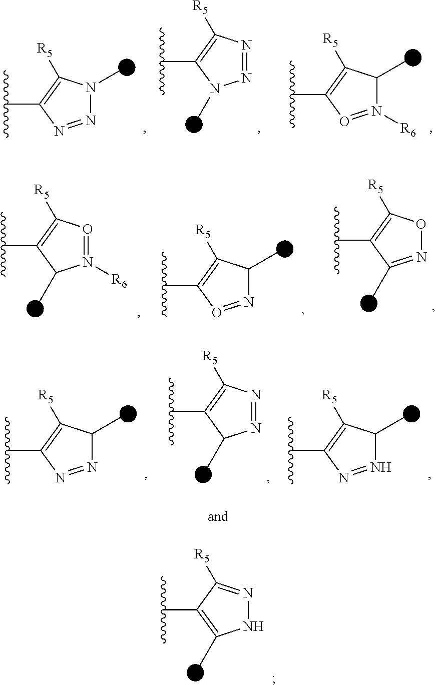

wherein R₅ is H or C₁-C₈-alkyl, preferably R₅ is H; R₆ is C₁-C₈-alkyl; and
● is as defined in any one of the preceding items;
preferably R₁ is

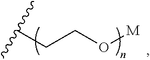

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4;
preferably Z is

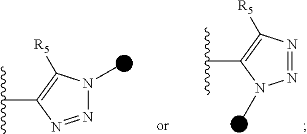

more preferably Z is

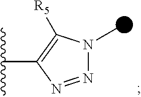

is H or C₁-C₈-alkyl, preferably R₅ is H; and ● is as defined in any one of the preceding items;
preferably in each instance Y is O;
optionally a linker is arranged between ● and Q.

31. The method according to any one of items 1 to 18 and 24, wherein R₁ is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

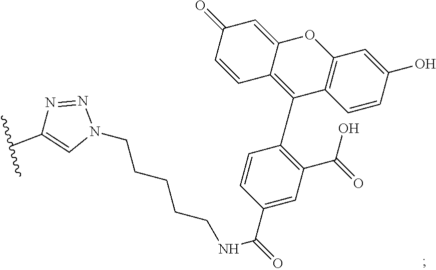 [SMILES: *c1cn(CCCCCNC(=O)c2ccc(-c3c4ccc(=O)cc-4oc4cc(O)ccc34)c(C(=O)O)c2)nn1]

or still more preferably R₁ is

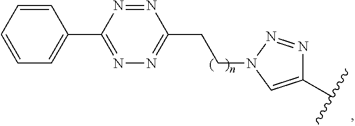

wherein n is 1, 2, 3, 4, 5, 6, 7, 8 or 9, preferably 1, 2 or 3, more preferably 1; or preferably R₁ is

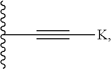

wherein K is H or C₁-C₈-alkyl, preferably K is H; and
Z is

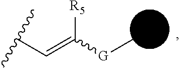

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

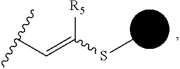

R₅ is H or C₁-C₈-alkyl, preferably R₅ is H, and ● is as defined in any one of the preceding items;
preferably in each instance Y is a bond;
optionally a linker is arranged between ● and Q.

32. The method according to any one of the preceding items, wherein
● represents an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q.

33. The method according to item 32, wherein
● represents an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q.

34. The method according to item 32 or 33, wherein ● represents an amino acid, a peptide, a protein, an antibody, a nucleotide or an oligonucleotide; wherein optionally a linker is arranged between ● and Q.

35. The method according to item 32, wherein ● represents a drug, a protein tag, a fluorophore such as CY₅, fluorescein or EDANS, biotin, a protein, a peptide, an antibody or an oligonucleotide; wherein optionally a linker is arranged between ● and Q.

36. The method according to item 32, wherein ● represents a linker, a drug, or a linker-drug conjugate.

37. The method according to item 32, wherein ● represents a linker, a fluorophore, or a linker-fluorophore conjugate.

38. The method according to item 32, wherein ● represents a small molecule, a fluorophore, a peptide, a protein, or an antibody; wherein optionally a linker is arranged between ● and Q.

39. The method according to any one of the preceding items, wherein [symbol] represents an amino acid, a peptide, a protein, an antibody, a nucleotide, an oligonucleotide, or a small molecule.

40. The method according to any one of the preceding items, wherein [symbol] represents an antibody, preferably an IgG antibody, more preferably a Cetuximab or a Trastuzumab or a Brentuximab; a protein, preferably a GFP protein, an eGFP-protein, an mCherry protein or an albumin; a small molecule; a peptide, preferably a peptide of formula (VIII)

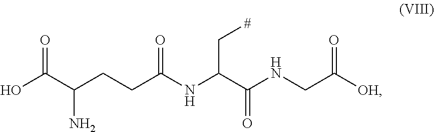

or of formula (IX)

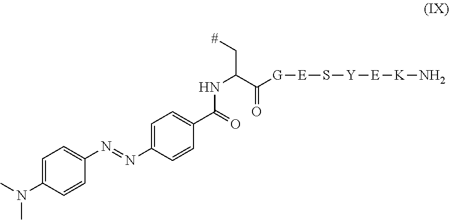

wherein #represents the position of S.

41. The method according to any one of items 1 to 32, wherein
[symbol] represents an antibody and
● represents a protein tag, or a fluorophore such as CY₅, fluorescein or EDANS, biotin, a peptide, a protein, an oligonucleotide, or a small molecule; wherein optionally
a linker is arranged between ● and Q.

42. The method according to any one of items 1 to 32, wherein
● represents a protein tag, or a fluorophore such as CY₅, fluorescein or EDANS, biotin, a peptide, an antibody, a protein, an oligonucleotide, or a small molecule;
wherein optionally a linker is arranged between ● and Q.

43. The method according to any one of items 1 to 32, wherein
[symbol] represents a peptide and
● represents a protein tag, or a fluorophore such as CY₅, fluorescein or EDANS, biotin, a peptide, a protein, an oligonucleotide, or a small molecule; wherein optionally
a linker is arranged between ● and Q.

44. The method according to any one of items 1 to 32, wherein
[symbol] represents an amino acid and
● represents a protein tag, or a fluorophore such as CY₅, fluorescein or EDANS, biotin, a peptide, a protein, an oligonucleotide, or a small molecule; wherein optionally
a linker is arranged between ● and Q.

45. The method according to any one of items 1 to 32, wherein
[symbol] represents an antibody and
● represents a linker, a drug, or a linker-drug conjugate.

46. The method according to any one of item 1 to 32, wherein
[symbol] represents an antibody and
● represents a linker, a fluorophore, or a linker-fluorophore conjugate.

47. The method according to any one of items 1 to 32, wherein represents a nucleotide and
● represents a peptide, a protein, a protein tag, an antibody, an oligonucleotide, a fluorophore such as CY₅, fluorescein or EDANS, biotin, or a small molecule; wherein
optionally a linker is arranged between ● and Q.

48. The method according to any one of items 1 to 32, wherein
[symbol] represents a nucleotide and
● epresents a linker.

49. The method according to any one of items 1 to 32, wherein
[symbol] represents an oligonucleotide and
● represents a peptide, a protein, a protein tag, an antibody, an oligonucleotide, a fluorophore such as CY₅, fluorescein or EDANS, biotin, or a small molecule; wherein
optionally a linker is arranged between ● and Q.

50. The method according to any one of items 1 to 32, wherein
[symbol] represents an oligonucleotide and
● epresents a linker.

51. The method according to any one of items 1 to 34, wherein ● represents an amino acid, a peptide, a nucleotide or an oligonucleotide, wherein the amino acid, peptide, nucleotide or oligonucleotide is bound to a solid support; wherein optionally a linker is arranged between ● and Q.

52. The method according to any one of items 1 to 39, wherein [symbol] represents an amino acid, a peptide, a nucleotide or an oligonucleotide, wherein the amino acid, peptide, nucleotide or oligonucleotide is bound to a solid support.

53. The method according to any one of items 1 to 50, wherein the

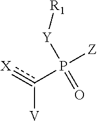

and the [symbol] -SH are in the same molecule.

54. A method of preparing a conjugate of an antibody molecule, said method comprising:
reducing at least one disulfide bridge of an antibody molecule in the presence of a reducing agent; and
reacting said antibody molecule with a compound of formula (IV*)

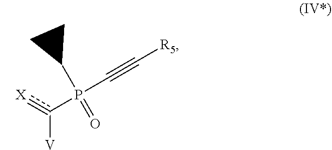

wherein
[symbol] represents a triple bond or a double bond;
V is absent when [symbol] is a triple bond; or
V represents H or C₁-C₈-alkyl when [symbol] is a double bond;
X represents R₃—C when [symbol] is a triple bond; or
X represent

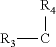

when [symbol] is a double bond;
▴ represents

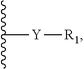

wherein [symbol] indicates the attachment point to the phosphorus; or
▴ represents Z; and
R₁, R₃, R₄, R₅, Y and Z are as defined in any one of the preceding items, resulting in a conjugate of an antibody molecule comprising at least one moiety of formula (V)

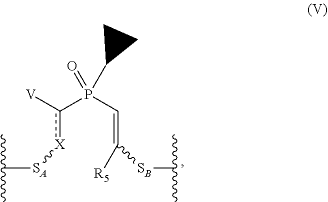

wherein S_{A} and S_{B} are each sulfur atoms of a chain of the antibody molecule;
[symbol] represents a double bond when [symbol] in a compound of formula (IV*) represents a triple bond; or
[symbol] represents a bond when [symbol] in a compound of formula (IV*) represents a double bond;
V is absent when [symbol] is a double bond; or
V represents H or C₁-C₈-alkyl when [symbol] is a bond;
X represents R₃—C when [symbol] is a double bond; or
X represents

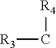

when [symbol] is a bond;
▴ represents

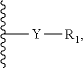

wherein [symbol] indicates the attachment point to the phosphorus; or
▴ represents Z; and
wherein R₁, R₃, R₄, R₅, Y and Z are as defined in any one of the preceding items.

55. The method according to item 54, wherein the antibody molecule is selected from the group consisting of an IgA, an IgD, an IgE, an IgG, an IgM, a human antibody, a humanized antibody, a chimeric antibody, a monoclonal antibody, and an isolated antibody.

56. The method according to item 55, wherein the antibody molecule is an IgG, such as e.g. a Trastuzumab, a Cetuximab or a Brentuximab.

57. The method according to any one of items 54 to 56, wherein the reducing agent is selected from the group consisting of tris(2-carboxyethyl)phosphine (TCEP), dithiothreitol (DTT), sodium dithionite, sodium thiosulfate, and sodium sulfite; preferably, the reducing agent is tris(2-carboxyethyl)phosphine (TCEP).

58. The method according to any one of items 54 to 57, wherein [symbol] represents a triple bond; V is absent; X represents R₃—C; R₃ represents H or C₁-C₈-alkyl, preferably R₃ is H; R₅ represents H or C₁-C₈-alkyl, preferably R₅ is H; and [symbol] represents a double bond.

59. The method according to any one of items 54 to 57, wherein [symbol] represents a double bond; V represents H or C₁-C₈-alkyl, preferably V is H; X represents

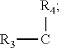

R₃ represents H or C₁-C₈-alkyl, preferably R₃ is H; R₄ represents H or C₁-C₈-alkyl, preferably R₄ is H; R₅ represents H or C₁-C₈-alkyl, preferably R₅ is H; and [symbol] represents a bond.

60. The method of any one of items 54 to 59, wherein ▴

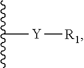

wherein [symbol] indicates the attachment point to the phosphorus; Y represents O, NR², S or a bond; R₁ represents an optionally substituted aliphatic or optionally substituted aromatic residue; and R² represents H or C₁-C₈-alkyl.

61. The method according to item 60, wherein Y is O.

62. The method according to item 60, wherein Y is NR²; preferably wherein R² is C₁-C₈-alkyl, more preferably wherein R² is methyl, ethyl, propyl or butyl, still more preferably wherein R² is methyl or ethyl.

63. The method according to item 60, wherein Y is S.

64. The method according to item 60, wherein Y is a bond.

65. The method according to any one of items 60 to 64, wherein R₁ represents a small molecule; C₁-C₈-alkyl optionally substituted with at least one of (C₁-C₈-alkoxy), wherein n is 1,2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; preferably in in each instance Y is O; or
R₁ represents phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; preferably in each instance Y is a bond; or
R₁ represents a 5- or 6-membered heteroaromatic system such as optionally substituted triazolyl or optionally substituted pyridyl; preferably in each instance Y is a bond.

66. The method according to item 65, wherein R₁ represents a small molecule, C₁-C₈-alkyl, C₁-C₈-alkyl substituted with —S—S—(C₁-C₈-alkyl), C₁-C₈-alkyl substituted with (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or Cr Ca-alkynyl; or phenyl; or phenyl substituted with —NO₂; or triazolyl substituted with optionally substituted C₁-C₈-alkyl; or triazolyl substituted with a fluorophore.

67. The method according to item 65 or 66, wherein R₁ represents C₁-C₈-alkyl, preferably methyl, ethyl, propyl or butyl, more preferably methyl or ethyl; still more preferably ethyl.

68. The method according to any one of items 60 to 64, wherein R₁ is selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O.

69. The method according to item 68, wherein R₁ is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

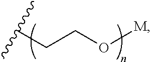

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

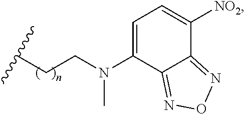

wherein n is 1, 2, 3, 4, 5, 6, 7, 8, 9 or 10, more preferably 4, 5 or 6, still more preferably 5, or more preferably R₁ is

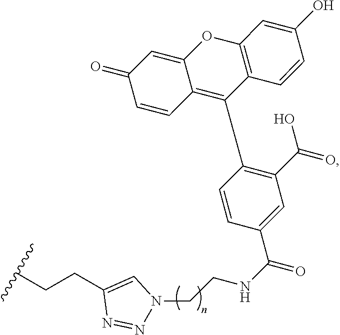

wherein n is 1, 2, 3, 4, 5, 6, 7, 8, 9 or 10, preferably 3, 4 or 5, still more preferably 4; C₂-C₈-alkynyl, preferably

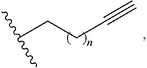

wherein n is 1, 2, 3, 4, or 5, preferably 1, 2 or 3, more preferably 1; or preferably R₁ is

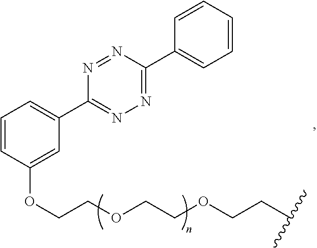

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

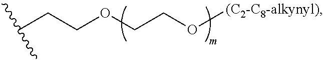

more preferably

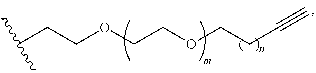

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O.

70. The method according to any one of items 60 to 64, wherein R₁ is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

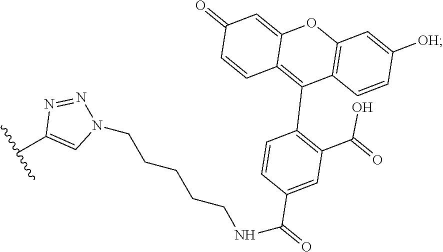 [SMILES: *c1cn(CCCCCNC(=O)c2ccc(-c3c4ccc(=O)cc-4oc4cc(O)ccc34)c(C(=O)O)c2)nn1]

or still more preferably R₁ is

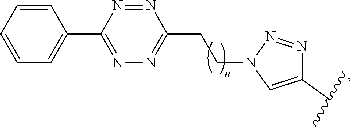

wherein n is 1, 2, 3, 4, 5, 6, 7, 8 or 9, preferably 1, 2 or 3, more preferably 1; or preferably R₁ is

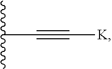

wherein K is H or C₁-C₈-alkyl, preferably K is H; preferably wherein in each instance Y is a bond.

71. The method according to any one of items 60 to 64, wherein R₁ represents an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between R₁ and Y.

72. The method according to item 71, wherein R₁ represents an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between R₁ and Y.

73. The method according to item 71 or 72, wherein R₁ represents an amino acid, a peptide, a protein, an antibody, a nucleotide or an oligonucleotide; wherein optionally a linker is arranged between R₁ and Y.

74. The method according to item 71, wherein R₁ represents a drug, a protein tag, or a fluorophore such as CY₅, fluorescein or EDANS, biotin, a protein, a peptide, an antibody or an oligonucleotide; wherein optionally a linker is arranged between R₁ and Y.

75. The method according to item 71, wherein R₁ represents a linker, a drug, or a linker-drug conjugate.

76. The method according to item 71, wherein R₁ represents a linker, a fluorophore, or a linker-fluorophore conjugate.

77. The method according to item 71, wherein R₁ represents a small molecule, a fluorophore, a peptide, a protein, or an antibody; wherein optionally a linker is arranged between R₁ and Y.

78. The method according to any one of items 54 to 59, wherein ▴ represents Z; and Z represents a residue bound to the phosphorus via a carbon atom and comprising a group ●, wherein ● represents an optionally substituted aliphatic or optionally substituted aromatic residue.

79. The method according to item 78, wherein Z is

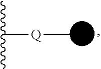

wherein [symbol] indicates the attachment point to the phosphorus and ● is as defined in any one of the preceding items; and
Q is a moiety comprising at least three main-chain atoms and a a carbon-carbon double bond, wherein at least one of the main-chain atoms is selected from the group consisting of S, O or N; wherein optionally a linker is arranged between ● and Q.

80. The method according to item 79, wherein Z is

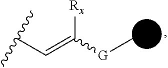

Rₓ is H or C₁-C₈-alkyl, preferably Rₓ is H; G is S, O or NR₁₀, wherein R₁₀ is H or C₁-C₈-alkyl, preferably R₁₀ is H; and ● is as defined in any one of the preceding items; wherein optionally a linker is arranged between ● and Q.

81. The method according to item 80, wherein Z is

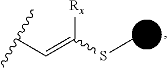

Rₓ is H or C₁-C₈-alkyl, preferably Rₓ is H, and ● is as defined in any one of the preceding items; wherein optionally a linker is arranged between ● and Q.

82. The method according to item 78, wherein Z is

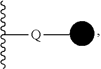

wherein [symbol] indicates the attachment point to the phosphorus and ● is as defined in any one of the preceding items; and
Q is a five- or six-membered heterocyclic moiety comprising 1, 2 or 3 heteroatoms independently selected from the group consisting of N, O or S; wherein optionally a linker is arranged between ● and Q.

83. The method according to item 82, wherein Z is selected from the group consisting of

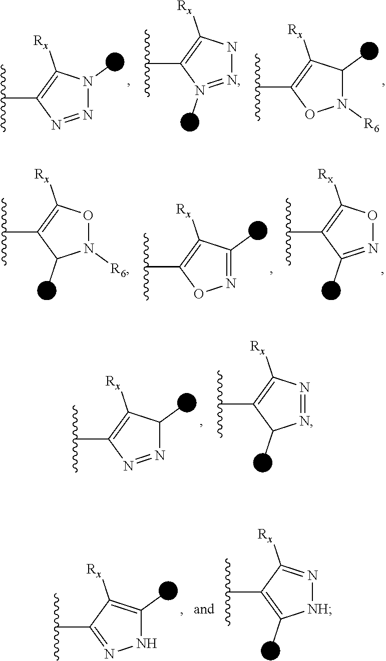

wherein Rₓ is H or C₁-C₈-alkyl, preferably Rₓ is H; R₆ is C₁-C₈-alkyl, and ● is as defined in any one of the preceding items; wherein optionally a linker is arranged between ● and Q.

84. The method according to item 83, wherein Z is

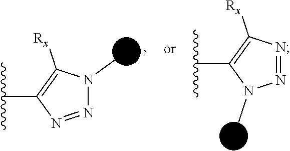

preferably Z is

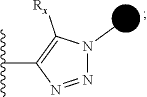

Rₓ is H or C₁-C₈-alkyl, preferably Rₓ is H, and ● is as defined in any one of the preceding items; wherein optionally a linker is arranged between ● and Q.

85. The method according to item 78, wherein Z is

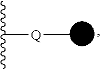

wherein [symbol] indicates the attachment point to the phosphorus and ● is as defined in any one of the preceding items; and
Q is a moiety comprising a carbon-carbon triple bond bound to the phosphorus in the compound of formula (IV*), and an optionally substituted phenyl group bound to the carbon-carbon triple bond; or
Q is a moiety comprising a carbon-carbon triple bond bound to the phosphorus in formula (IV*), and an optionally substituted carbon-carbon double bond bound to the carbon-carbon triple bond;
wherein optionally a linker is arranged between ● and Q.

86. The method according to item 85, wherein Z is

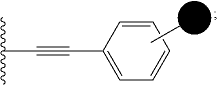

or wherein Z is

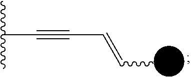

wherein ● is as defined in any one of the preceding items; wherein optionally a linker is arranged between ● and Q.

87. The method according to any one of items 78 to 86, wherein ● represents an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q.

88. The method according to item 87, wherein ● represents an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q.

89. The method according to item 87 or 88, wherein ● represents an amino acid, a peptide, a protein, an antibody, a nucleotide or an oligonucleotide; wherein optionally a linker is arranged between ● and Q.

90. The method according to item 87, wherein 4 represents a drug, a protein tag, or a fluorophore such as CY₅, fluorescein or EDANS, biotin, a protein, a peptide, an antibody or an oligonucleotide; wherein optionally a linker is arranged between ● and Q.

91. The method according to item 87, wherein ● represents a linker, a drug, or a linker-drug conjugate.

92. The method according to item 87, wherein ● represents a linker, a fluorophore, or a linker-fluorophore conjugate.

93. The method according to item 87, wherein 4 represents a small molecule, a fluorophore, a peptide, a protein, or an antibody; wherein optionally a linker is arranged between ● and Q.

94. The method according to any one of items 78 to 86, wherein ● represents a small molecule; C₁-C₈-alkyl optionally substituted with at least one of (C₁-C₈-alkoxy), wherein n is 1,2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈alkyl)H, —NH₂, —N₃, —N(C₁-C₈alkyl)₂, ═O, C₃-C₈-cycloalkyl, —S—S—(C₁-C₈-alkyl), hydroxy-(C₁—C-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; wherein optionally a linker is arranged between* and Q; or
● represents phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈alkyl)H, —NH₂, —N(C₁-C₈-alkyl)₂, or hydroxy-(C₁—C-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30;
wherein optionally a linker is arranged between ● and Q; or
● represents a 5- or 6-membered heteroaromatic system such as optionally substituted triazolyl or optionally substituted pyridyl; wherein optionally a linker is arranged between ● and Q.

95. The method according to item 94, wherein ● represents a small molecule, C₁-C₈-alkyl, C₁-C₈-alkyl substituted with —S—S—(C₁-C₈-alkyl), C₁-C₈-alkyl substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or Cr Ca-alkynyl; or phenyl; or phenyl substituted with —NO₂; or triazolyl substituted with optionally substituted C₁-C₈-alkyl; or triazolyl substituted with a fluorophore; wherein optionally a linker is arranged between ● and Q.

96. The method according to item 94 or 95, wherein ● represents C₁-C₈-alkyl, preferably methyl, ethyl, propyl or butyl, more preferably methyl or ethyl; still more preferably ethyl; wherein optionally a linker is arranged between ● and Q.

97. The method according to any one of items 78 to 86, wherein ● is selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; wherein optionally a linker is arranged between ● and Q.

98. The method according to item 97, wherein 4 is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably ● is

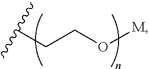

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably ● is

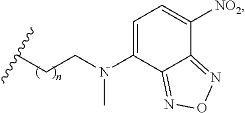

wherein n is 1, 2, 3, 4, 5, 6, 7, 8, 9 or 10, more preferably 4, 5 or 6, still more preferably 5, or more preferably ● is

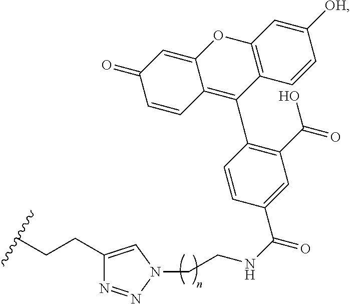

wherein n is 1, 2, 3, 4, 5, 6, 7, 8, 9 or 10, preferably 3, 4 or 5, still more preferably 4; C₂-C₈-alkynyl, preferably

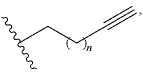

wherein n is 1, 2, 3, 4, or 5, preferably 1, 2 or 3, more preferably 1; or preferably ● is

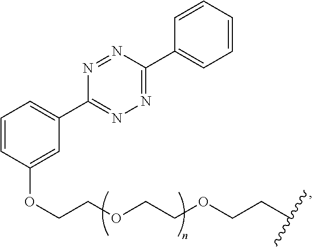

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably ● is

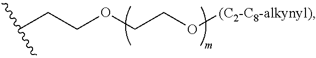

more preferably

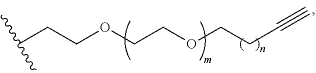

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; wherein optionally a linker is arranged between ● and Q.

99. The method according to any one of items 78 to 86, wherein ● is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably ● is

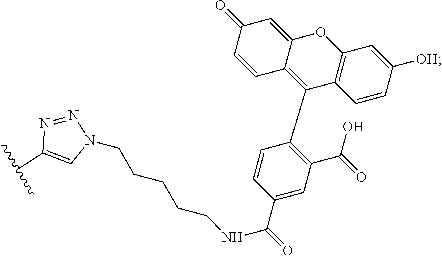 [SMILES: *c1cn(CCCCCNC(=O)c2ccc(-c3c4ccc(=O)cc-4oc4cc(O)ccc34)c(C(=O)O)c2)nn1]

or still more preferably ● is

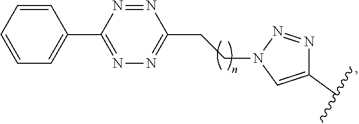

wherein n is 1, 2, 3, 4, 5, 6, 7, 8 or 9, preferably 1, 2 or 3, more preferably 1; or preferably ● is

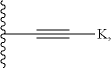

wherein K is H or C₁-C₈-alkyl, preferably K is H; wherein optionally a linker is arranged between ● and Q.

100. A compound of formula (I)

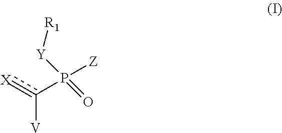

wherein [symbol] , R₁, V, X, Y and Z are as defined in any one of the preceding items, in particular as defined in any one of items 1 to 53.

101. The compound according to item 100, wherein [symbol] represents a triple bond; V is absent; X represents R₃—C; and R₃ represents H or C₁-C₈-alkyl, preferably R₃ is H.

102. The compound according to item 100, wherein [symbol] represents a double bond; V is H or C₁-C₈-alkyl, preferably V is H; X represents

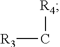

R₃ represents H or C₁-C₈-alkyl, preferably R₃ is H; and R₄ represents H or C₁-C₈-alkyl, preferably R₄ is H.

103. A compound of formula (III)

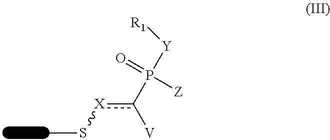

wherein
[symbol] represents a double bond; or
[symbol] represents a bond;
V is absent when [symbol] is a double bond; or
V represents H or C₁-C₈-alkyl when [symbol] is a bond;
X represents R₃—C when [symbol] is a double bond; or
X represents

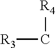

when [symbol] is a bond; and
R₁, R₃, R₄, Y, Z and [symbol] are as defined in any one of the preceding items, in particular as defined in any one of items 1 to 53.

104. The compound according to item 103, wherein [symbol] represents a double bond; V is absent; X represents R₃—C; and R₃ is H or C₁-C₈-alkyl, preferably R₃ is H.

105. The compound according to item 103, wherein [symbol] represents a bond; V represents H or C₁-C₈-alkyl, preferably V is H; X represents

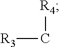

R₃ represents H or C₁-C₈-alkyl, preferably R₃ is H; R₄ represents H or C₁-C₈-alkyl, preferably R₄ is H.

106. The compound according to any one of items 100 to 105, wherein Z is

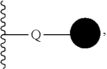

wherein [symbol] indicates the attachment point to the phosphorus and ● is as defined in any one of the preceding items; and
Q is a moiety comprising at least three main-chain atoms and a carbon-carbon double bond, wherein at least one of the main-chain atoms is selected from the group consisting of S, O or N; wherein optionally a linker is arranged between ● and Q.

107. The compound according to item 106, wherein Z is

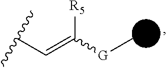

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items; wherein optionally a linker is arranged between ● and Q.

108. The compound according to item 107, wherein Z is

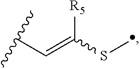

R₅ is H or C₁-C₈-alkyl, preferably R₅ is H, and ● is as defined in any one of the preceding items; wherein optionally a linker is arranged between ● and Q.

109. The compound according to any one of items 100 to 105, wherein Z is

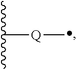

wherein [symbol] indicates the attachment point to the phosphorus and ● is as defined in any one of the preceding items; and
Q is a five- or six-membered heterocyclic moiety comprising 1, 2 or 3 heteroatoms independently selected from the group consisting of N, O or S; wherein optionally a linker is arranged between ● and Q.

110. The compound according to item 109, wherein Z is selected from the group consisting of

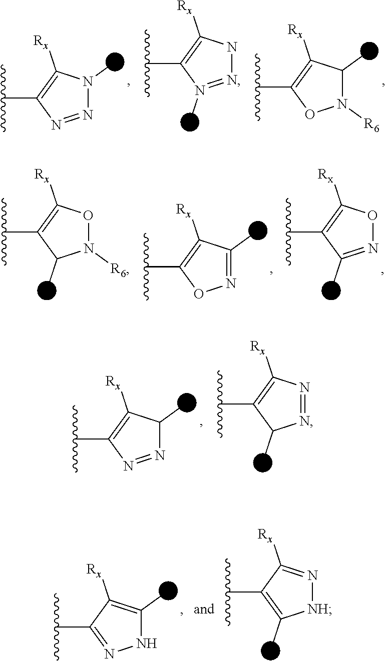

wherein R₅ is H or C₁-C₈-alkyl, preferably R₅ is H; Ra is C₁-C₈-alkyl, and ● is as defined in any one of the preceding items; wherein optionally a linker is arranged between ● and Q.

111. The compound according to item 110, wherein Z is

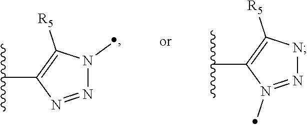

preferably Z is

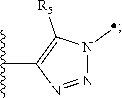

R₅ is H or C₁-C₈-alkyl, preferably R₅ is H, and ● is as defined in any one of the preceding items; wherein optionally a linker is arranged between ● and Q.

112. The compound according to any one of items 100 to 105, wherein Z is

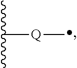

wherein [symbol] indicates the attachment point to the phosphorus and ● is as defined in any one of the preceding items; and
Q is a moiety comprising a carbon-carbon triple bond bound to the phosphorus in the compound of formula (I) or formula (III), and an optionally substituted phenyl group bound to the carbon-carbon triple bond; or
Q is a moiety comprising a carbon-carbon triple bond bound to the phosphorus in the compound of formula (I) or formula (III), and an optionally substituted carbon-carbon double bond bound to the carbon-carbon triple bond;
wherein optionally a linker is arranged between ● and Q.

113. The compound according to item 112, wherein Z is

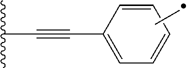

or wherein Z is

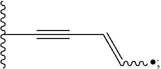

wherein ● is as defined in any one of the preceding items; wherein optionally a linker is arranged between ● and Q.

114. The compound according to any one of items 100 to 113, wherein Y is O.

115. The compound according to any one of items 100 to 113, wherein Y is NR²; preferably wherein R² is C₁-C₈-alkyl, more preferably wherein R² is methyl, ethyl, propyl or butyl, still more preferably wherein R² is methyl or ethyl.

116. The compound according to any one of items 100 to 113, wherein Y is S.

117. The compound according to any one of items 100 to 113, wherein Y is a bond.

118. The compound according to any one of items 100 to 117, wherein R₁ represents a small molecule; C₁-C₈-alkyl optionally substituted with at least one of (C₁-C₈-alkoxy), wherein n is 1,2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; preferably in in each instance Y is O; or
R₁ represents phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; preferably in each instance Y is a bond; or
R₁ represents a 5- or 6-membered heteroaromatic system such as optionally substituted triazolyl or optionally substituted pyridyl; preferably in each instance Y is a bond.

119. The compound according to item 118, wherein R₁ represents a small molecule, C₁-C₈-alkyl, C₁-C₈-alkyl substituted with —S—S—(C₁-C₈-alkyl), C₁-C₈-alkyl substituted with (C₁—Ca-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore.

120. The compound according to item 118 or 119, wherein R₁ represents C₁-C₈-alkyl, preferably methyl, ethyl, propyl or butyl, more preferably methyl or ethyl, still more preferably ethyl.

121. The compound according to any one of items 100 to 117, wherein R₁ is selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O.

122. The compound according to item 121, wherein R₁ is selected from the group consisting of ethyl; C₁-C₈alkyl optionally substituted with (C₁-C₈alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

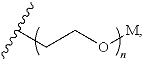

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

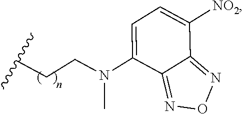

wherein n is 1, 2, 3, 4, 5, 6, 7, 8, 9 or 10, more preferably 4, 5 or 6, still more preferably 5, or more preferably R₁ is

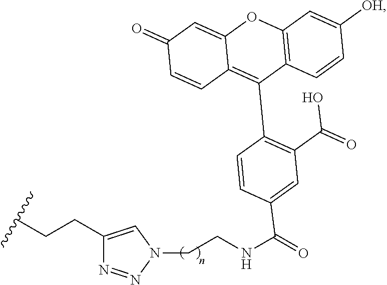

wherein n is 1, 2, 3, 4, 5, 6, 7, 8, 9 or 10, preferably 3, 4 or 5, still more preferably 4; C₂-C₈-alkynyl, preferably

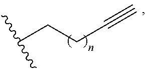

wherein n is 1, 2, 3, 4, or 5, preferably 1, 2 or 3, more preferably 1; or preferably R₁ is

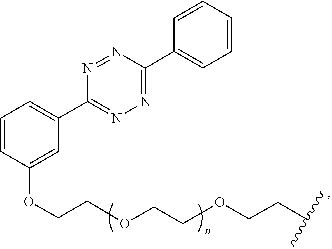

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

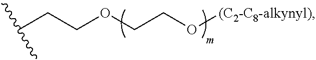

more preferably

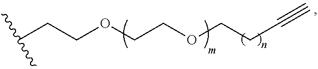

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O.

123. The compound according to any one of items 100 to 117, wherein R₁ is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

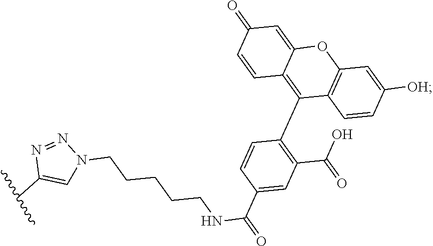 [SMILES: *c1cn(CCCCCNC(=O)c2ccc(-c3c4ccc(=O)cc-4oc4cc(O)ccc34)c(C(=O)O)c2)nn1]

or still more preferably R₁ is

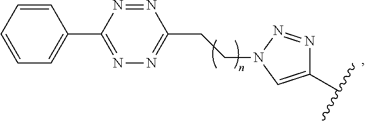

wherein n is 1, 2, 3, 4, 5, 6, 7, 8 or 9, preferably 1, 2 or 3, more preferably 1; or preferably R₁ is

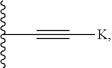

wherein K is H or C₁-C₈-alkyl, preferably K is H; preferably wherein in each instance Y is a bond.

124. The compound according to any one of items 100 to 122, wherein R₁ is C₁-C₈-alkyl, preferably methyl, ethyl, propyl or butyl; more preferably methyl or ethyl; still more preferably ethyl; and
Z is

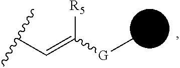

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

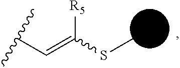

R₅ is H or C₁-C₈-alkyl, preferably R₅ is H, and ● is as defined in any one of the preceding items;
preferably in each instance Y is O;
optionally the C₁-C₈-alkyl is substituted with a fluorophore
optionally a linker is arranged between ● and Q.

125. The compound according to any one of items 100 to 122, wherein R₁ is C₂-C₈-alkynyl, preferably

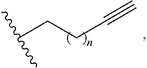

wherein n is 1, 2, 3, 4, or 5, preferably 1, 2 or 3, more preferably 1; or wherein R₁ is

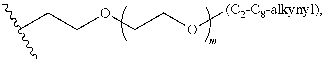

preferably

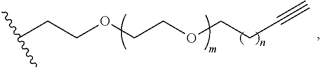

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and
Z is

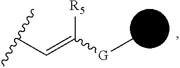

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

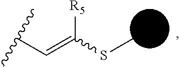

R₅ is H or C₁-C₈-alkyl, preferably R₅ is H, and ● is as defined in any one of the preceding items;
preferably in each instance Y is O;
optionally a linker is arranged between ● and Q.

126. The compound according to any one of items 100 to 122, wherein R₁ is C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and
Z is

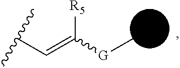

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

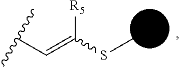

R₅ is H or C₁-C₈-alkyl, preferably R₅ is H, and ● is as defined in any one of the preceding items;
preferably R₁ is

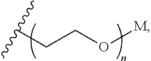

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4;
preferably in each instance Y is O;
optionally a linker is arranged between ● and Q.

127. The compound according to any one of items 100 to 122, wherein R₁ is C₁-C₈-alkyl, preferably methyl, ethyl, propyl or butyl; more preferably methyl or ethyl; still more preferably ethyl; and
Z is selected from the group consisting of

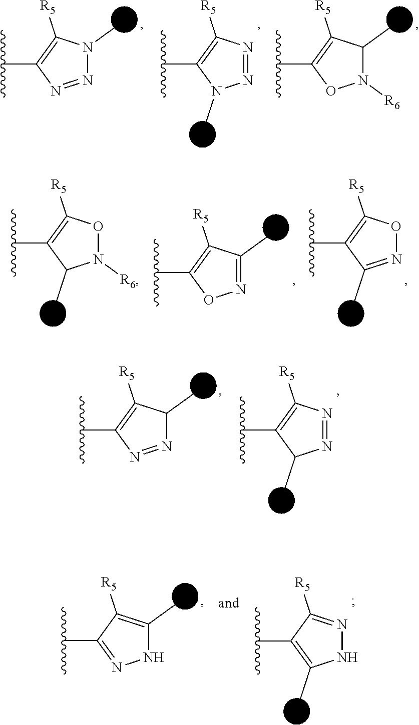

wherein R₅ is H or C₁-C₈-alkyl, preferably R₅ is H; R₆ is C₁-C₈-alkyl; and ● is as defined in any one of the preceding items;
preferably Z is

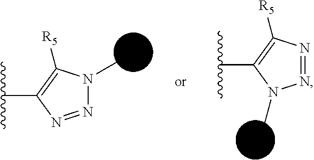

more preferably Z is

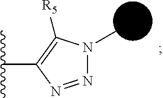

R₅ is H or C₁-C₈alkyl, preferably R₅ is H; and ● is as defined in any one of the preceding items;
preferably in each instance Y is O;
optionally the C₁-C₈-alkyl is substituted with a fluorophore;
optionally a linker is arranged between ● and Q.

128. The compound according to any one of items 100 to 122, wherein R₁ is C₂-C₈-alkynyl, preferably

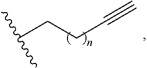

wherein n is 1, 2, 3, 4, 5, 6, 7, 8, 9 or 10, preferably 1, 2 or 3, more preferably 1; or wherein R₁ is

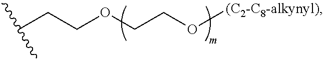

preferably

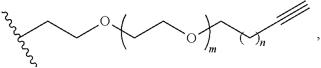

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; and
Z is selected from the group consisting of

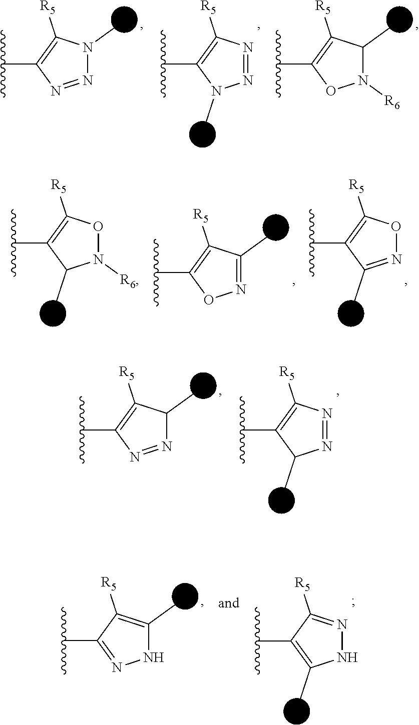

wherein R₅ is H or C₁-C₈-alkyl, preferably R₅ is H; R₆ is C₁-C₈-alkyl; and ● is as defined in any one of the preceding items;
preferably Z is

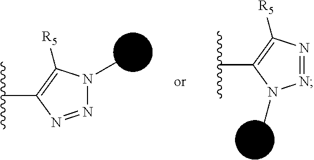

more preferably Z is

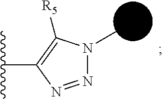

R₅ is H or C₁-C₈alkyl, preferably R₅ is H; and ● is as defined in any one of the preceding items;
preferably in each instance Y is O;
optionally a linker is arranged between ● and Q.

129. The compound according to any one of items 100 to 122, wherein R₁ is C₁-C₈-alkyl optionally substituted with at least one of (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Z is selected from the group consisting of

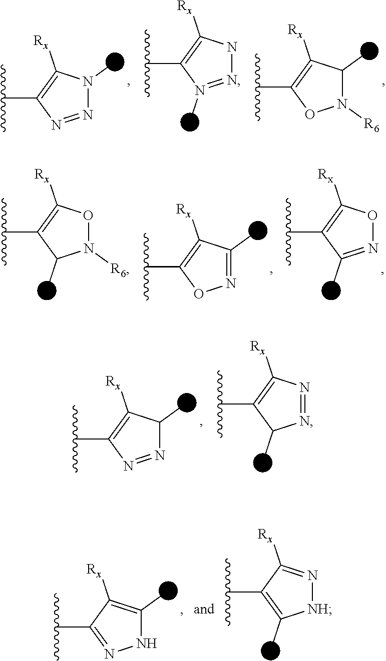

wherein R₅ is H or C₁-C₈-alkyl, preferably R₅ is H; R₆ is C₁-C₈-alkyl; and
● is as defined in any one of the preceding items;
preferably R₁ is

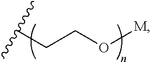

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4;
preferably Z is

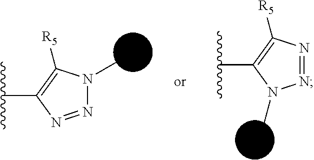

or more preferably Z is

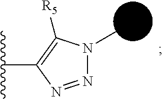

R₅ is H or C₁-C₈-alkyl, preferably R₅ is H; and ● is as defined in any one of the preceding items;
preferably in each instance Y is O;
optionally a linker is arranged between ● and Q.

130. The compound according to any one of items 100 to 117 and 123, wherein R₁ is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

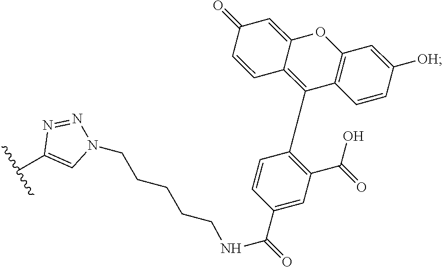 [SMILES: *c1cn(CCCCCNC(=O)c2ccc(-c3c4ccc(=O)cc-4oc4cc(O)ccc34)c(C(=O)O)c2)nn1]

or still more preferably R₁ is

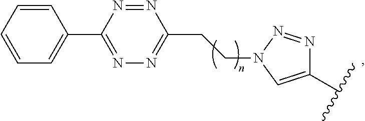

wherein n is 1, 2, 3, 4, 5, 6, 7, 8 or 9, preferably 1, 2 or 3, more preferably 1; or preferably wherein R₁ is

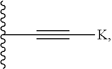

wherein K is H or C₁-C₈-alkyl, preferably K is H; and
Z is

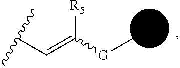

R₅ is H or C₁-C₈-alkyl, preferably R₅ is H; G is S, O or NR₁₀, wherein R₁₀ is H or C₁-C₈-alkyl, preferably R₁₀ is H; and ● is as defined in any one of the preceding items;
preferably, Z is

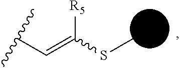

R₅ is H or C₁-C₈-alkyl, preferably R₅ is H, and ● is as defined in any one of the preceding items;
preferably in each instance Y is a bond;
optionally a linker is arranged between ● and Q.

131. The compound according to any one of items 100 to 130, wherein ● represents an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q.

132. The compound according to item 131, wherein ● represents an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q.

133. The compound according to item 131 or 132, wherein ● represents an amino acid, a peptide, a protein, an antibody, a nucleotide or an oligonucleotide; wherein optionally a linker is arranged between ● and Q.

134. The compound according to item 131, wherein ● represents a drug, a protein tag, a fluorophore such as CY₅, fluorescein or EDANS, biotin, a protein, a peptide, an antibody or an oligonucleotide; wherein optionally a linker is arranged between ● and Q.

135. The compound according to item 131, wherein ● represents a linker, a drug, or a linker-drug conjugate.

136. The compound according to item 131, wherein 4 represents a linker, a fluorophore, or a linker-fluorophore conjugate.

137. The compound according to item 131, wherein ● represents a small molecule, a fluorophore, a peptide, a protein, or an antibody; wherein optionally a linker is arranged between ● and Q.

138. The compound according to any one of items 103 to 137, wherein [symbol] represents an amino acid, a peptide, a protein, an antibody, a nucleotide, an oligonucleotide, or a small molecule.

139. The compound according to any one of items 103 to 138, wherein [symbol] represents an antibody, preferably an IgG antibody, more preferably a Cetuximab or a Trastuzumab or a Brentuximab; a protein, preferably a GFP protein, an eGFP-protein, an mCherry protein or an albumin; a small molecule; a peptide, preferably a peptide of formula (VIII)

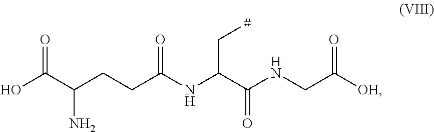

or of formula (IX)

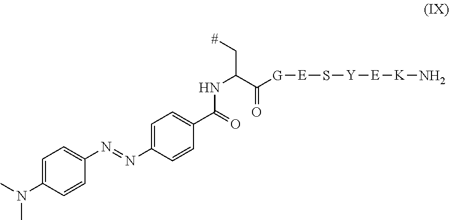

wherein #represents the position of S.

140. The compound according to any one of items 103 to 131, wherein
[symbol] represents an antibody and
● represents a protein tag, or a fluorophore such as CY₅, fluorescein or EDANS, biotin, a peptide, a protein, an oligonucleotide, or a small molecule; wherein optionally a linker is arranged between ● and Q.

141. The compound according to any one of items 103 to 131, wherein
[symbol] represents a protein and
● represents a protein tag, or a fluorophore such as CY₅, fluorescein, or EDANS, biotin, a peptide, an antibody, a protein, an oligonucleotide, or a small molecule;
wherein optionally a linker is arranged between ● and Q.

142. The compound according to any one of items 103 to 131, wherein
[symbol] represents a peptide and
● represents a protein tag, or a fluorophore such as CY₅, fluorescein or EDANS, biotin, a peptide, a protein, an oligonucleotide, or a small molecule; wherein optionally a linker is arranged between ● and Q.

143. The compound according to any one of items 103 to 131, wherein
[symbol] represents an amino acid and
● represents a protein tag, or a fluorophore such as CY₅, fluorescein or EDANS, biotin, a peptide, a protein, an oligonucleotide, or a small molecule; wherein optionally
a linker is arranged between ● and Q.

144. The compound according to any one of items 103 to 131, wherein
[symbol] represents an antibody and
● represents a linker, a drug, or a linker-drug conjugate.

145. The compound according to any one of items 103 to 131, wherein
[symbol] represents an antibody and
● represents a linker, a fluorophore, or a linker-fluorophore conjugate.

146. The compound according to any one of items 103 to 131, wherein
[symbol] represents a nucleotide and
● represents a peptide, a protein, a protein tag, an antibody, an oligonucleotide, a fluorophore such as CY₅, fluorescein or EDANS, biotin, or a small molecule; wherein optionally a linker is arranged between ● and Q.

147. The compound according to any one of items 103 to 131, wherein
[symbol] represents a nucleotide and
● represents a linker.

148. The compound according to any one of items 103 to 131, wherein
[symbol] represents an oligonucleotide and
● represents a peptide, a protein, a protein tag, an antibody, an oligonucleotide, a fluorophore such as CY₅, fluorescein or EDANS, biotin, or a small molecule; wherein
optionally a linker is arranged between ● and Q.

149. The compound according to any one of items 103 to 131, wherein
[symbol] represents an oligonucleotide and
● represents a linker.

150. The compound according to any one of items 100 to 133, wherein ● represents an amino acid, a peptide, a nucleotide or an oligonucleotide, wherein the amino acid, peptide, nucleotide or oligonucleotide is bound to a solid support, wherein optionally a linker is arranged between ● and Q.

151. The compound according to any one of items 103 to 138, wherein [symbol] represents an amino acid, a peptide, a nucleotide or an oligonucleotide, wherein the amino acid, peptide, nucleotide or oligonucleotide is bound to a solid support.

152. A compound of formula (IIIa)

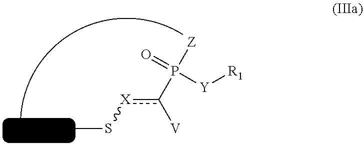

wherein
[symbol] represents a double bond; or
[symbol] represents a bond;
V is absent when [symbol] is a double bond; or
V represents H or C₁-C₈-alkyl when [symbol] is a bond;
X represents R₃—C when [symbol] is a double bond; or
X represents

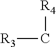

when [symbol] is a bond; and
R₁, R₃, R₄, Y, Z and [symbol] are as defined in any one of the preceding items, in particular as defined in any one of items 1 to 53 or 100 to 151.

153. A compound of formula (IV)

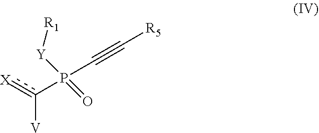

wherein R₁, R₅, [symbol] , V, X and Y are as defined in any one of the preceding items, in particular as defined in any one of items 1 to 52; and/or in particular as defined in any one of items 100 to 152.

154. A compound of formula (IV*)

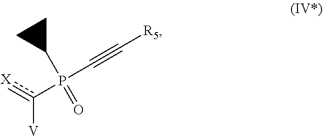

wherein
[symbol] represents a triple bond or a double bond;
V is absent when [symbol] is a triple bond; or
V represents H or C₁-C₈-alkyl when [symbol] is a double bond;
X represents R₃—C when [symbol] is a triple bond;
X represents

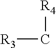

when [symbol] is a double bond;
▴ represents

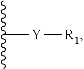

[symbol]

-Y—R₁, wherein [symbol] indicates the attachment point to the phosphorus; or
▴ represents Z; and
R₁, R₃, R₄, R₅, Y and Z are as defined in any one of the preceding items, in particular as defined in any one of items 54 to 99.

155. The compound according to item 154, wherein [symbol] represents a triple bond; V is absent; X represents R₃—C; R₃ represents H or C₁-C₈-alkyl, preferably R₃ is H; and R₅ is H or C₁-C₈-alkyl, preferably R₅ is H.

156. The compound according to item 154, wherein [symbol] represents a double bond; V represents H or C₁-C₈-alkyl, preferably V is H; X represents

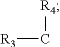

R₃ represents H or C₁-C₈-alkyl, preferably R₃ is H; R₄ represents H or C₁-C₈-alkyl, preferably R₄ is H; and R⁵ is H or C₁-C₈-alkyl, preferably R⁵ is H.

157. A conjugate of an antibody molecule comprising at least one moiety of formula (V)

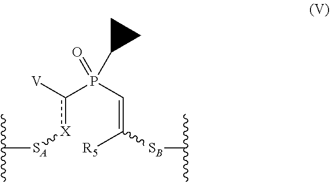

wherein S_{A} and S_{B} are each sulfur atoms of a chain of the antibody molecule;
[symbol] represents a double bond; or
[symbol] represents a bond;
V is absent when [symbol] is a double bond; or
V represents H or C₁-C₈-alkyl when [symbol] is a bond;
X represents R₃—C when [symbol] is a double bond; or
X represents

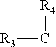

when [symbol] is a bond;
▴ represents

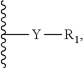

wherein [symbol] indicates the attachment point to the phosphorus; or
▴ represents Z; and
R₁, R₃, R₄, R₅, Y and Z are as defined in any one of the preceding items, in particular as defined in any one of items 54 to 99.

158. The conjugate of an antibody molecule according to item 157, wherein the antibody molecule is selected from the group consisting of an IgA, an IgD, an IgE, an IgG, an IgM, a human antibody, a humanized antibody, a chimeric antibody, a monoclonal antibody, and an isolated antibody.

159. The conjugate of an antibody molecule according to item 158, wherein the antibody molecule is an IgG, preferably a Trastuzumab, a Cetuximab or a Brentuximab; or a fragment thereof.

160. The conjugate of an antibody molecule according to any one of items 157 to 159, wherein [symbol] represents a double bond; V is absent; X represents R₃C; R₃ is H or C₁-C₈-alkyl, preferably R₃ is H; and R₅ is H or C₁-C₈-alkyl, preferably R₅ is H.

161. The conjugate of an antibody molecule according to any one of items 157 to 160, wherein [symbol] represents a bond; V represents H or C₁-C₈-alkyl, preferably V is H; X represents

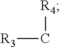

R₃ represents H or C₁-C₈-alkyl, preferably R₃ is H; R₄ represents H or C₁-C₈-alkyl, preferably R₄ is H; and R₅ is H or C₁-C₈-alkyl, preferably R₅ is H.

162. The compound according to any one of items 154 to 156, or the conjugate of an antibody molecule according to any one of items 157 to 161, wherein ▴ represents

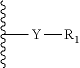

wherein [symbol] indicates the attachment point to the phosphorus; Y represents O, NR², S or a bond; R₁ represents an optionally substituted aliphatic or optionally substituted aromatic residue; and R² represents H or C₁-C₈-alkyl.

163. The compound according to item 162, or the conjugate of an antibody molecule according to item 162, wherein Y is O.

164. The compound according to item 162, or the conjugate of an antibody molecule according to item 162, wherein Y is NR²; preferably wherein R² is C₁-C₈-alkyl, more preferably wherein R² is methyl, ethyl, propyl or butyl, still more preferably wherein R² is methyl or ethyl.

165. The compound according to item 162, or the conjugate of an antibody molecule according to item 162, wherein Y is S.

166. The compound according to item 162, or the conjugate of an antibody molecule according to item 162, wherein Y is a bond.

167. The compound according to any one of items 162 to 166, or the conjugate of an antibody molecule according to any one of items 162 to 166, wherein R₁ represents a small molecule; C₁-C₈-alkyl optionally substituted with at least one of (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, C₂-C₈-alkenyl; C₂-C₈-alkynyl; preferably in in each instance Y is O; or
R₁ represents phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, CI, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, preferably in each instance Y is a bond; or
R₁ represents a 5- or 6-membered heteroaromatic system such as optionally substituted triazolyl or optionally substituted pyridyl; preferably in each instance Y is a bond.

168. The compound according to item 167, or the conjugate of an antibody molecule according to item 167, wherein R₁ represents a small molecule, C₁-C₈-alkyl, C₁-C₈-alkyl substituted with —S—S—(C₁-C₈-alkyl), C₁-C₈-alkyl substituted with (C₁-C₈-alkoxy), wherein n is 1,2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore.

169. The compound according to item 167 or 168, or the conjugate of an antibody molecule according to item 167 or 168, wherein R₁ represents C₁-C₈-alkyl, preferably methyl, ethyl, propyl or butyl, more preferably methyl or ethyl; still more preferably ethyl.

170. The compound according to any one of items 162 to 166, or the conjugate of an antibody molecule according to any one of items 162 to 166, wherein R₁ is selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; preferably wherein in each instance Y is O.

171. The compound according to item 170, or the conjugate of an antibody molecule according to item 170, wherein R₁ is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably R₁ is

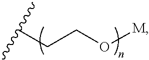

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably R₁ is

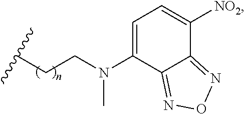

wherein n is 1, 2, 3, 4, 5, 6, 7, 8, 9 or 10, more preferably 4, 5 or 6, still more preferably 5, or more preferably R₁ is

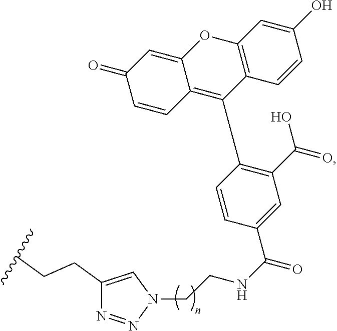

wherein n is 1, 2, 3, 4, 5, 6, 7, 8, 9 or 10, preferably 3, 4 or 5, still more preferably 4; C₂-C₈-alkynyl, preferably

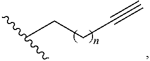

wherein n is 1, 2, 3, 4, or 5, preferably 1, 2 or 3, more preferably 1; or preferably R₁ is

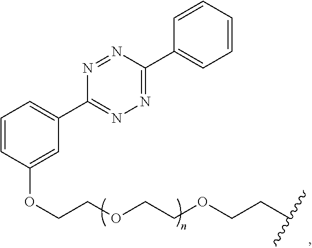

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably R₁ is

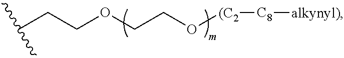

more preferably

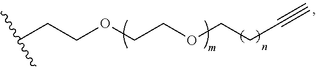

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; preferably wherein in each instance Y is O.

172. The compound according to any one of items 162 to 166, or the conjugate of an antibody molecule according to any one of items 162 to 166, wherein R₁ is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably R₁ is

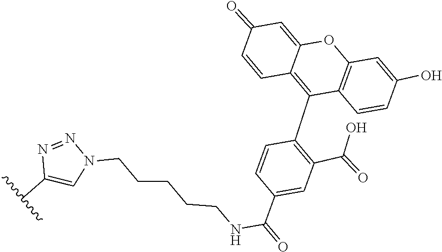 [SMILES: *c1cn(CCCCCNC(=O)c2ccc(-c3c4ccc(=O)cc-4oc4cc(O)ccc34)c(C(=O)O)c2)nn1]

or still more preferably R₁ is

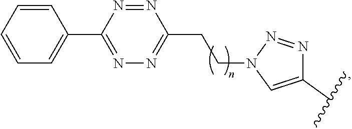

wherein n is 1, 2, 3, 4, 5, 6, 7, 8 or 9, preferably 1, 2 or 3, more preferably 1; or preferably R₁ is

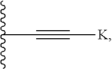

wherein K is H or C₁-C₈-alkyl, preferably K is H; preferably wherein in each instance Y is a bond.

173. The compound according to any one of items 162 to 166, or the conjugate of an antibody molecule according to any one of items 162 to 166, wherein R₁ represents an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between R₁ and Y.

174. The compound according to item 173, or the conjugate of an antibody molecule according to item 173, wherein R₁ represents an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between R₁ and Y.

175. The compound according to item 173 or item 174, or the conjugate of an antibody molecule according to item 173 or 174, wherein R₁ represents an amino acid, a peptide, a protein, an antibody, a nucleotide or an oligonucleotide; wherein optionally a linker is arranged between R₁ and Y.

176. The compound according to item 173, or the conjugate of an antibody molecule according to item 173, wherein R₁ represents a drug, a protein tag, or a fluorophore such as CY₅, fluorescein or EDANS, biotin, a protein, a peptide, an antibody or an oligonucleotide; wherein optionally a linker is arranged between R₁ and Y.

177. The compound according to item 173, or the conjugate of an antibody according to item 173, wherein R₁ represents a linker, a drug, or a linker-drug conjugate.

178. The compound according to item 173, or the conjugate of an antibody molecule according to item 173, wherein R₁ represents a linker, a fluorophore, or a linker-fluorophore conjugate.

179. The compound according to item 173, or the conjugate of an antibody molecule according to item 173, wherein R₁ represents a small molecule, a fluorophore, a peptide, a protein, or an antibody; wherein optionally a linker is arranged between R₁ and Y.

180. The compound according to any one of items 154 to 156, or the conjugate of an antibody molecule according to any one of items 154 to 156, wherein ▴ represents Z; and Z represents a residue bound to the phosphorus via a carbon atom and comprising a group ●, wherein ● represents an optionally substituted aliphatic or optionally substituted aromatic residue.

181. The compound according to item 180, or the conjugate of an antibody molecule according to item 180, wherein Z is

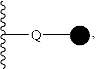

wherein [symbol] indicates the attachment point to the phosphorus and ● is as defined in any one of the preceding items; and Q is a moiety comprising at least three main-chain atoms and a carbon-carbon double bond, wherein at least one of the main-chain atoms is selected from the group consisting of S, O or N; wherein optionally a linker is arranged between ● and Q.

182. The compound according to item 181, or the conjugate of an antibody molecule according to item 181, wherein Z is

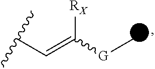

Rₓ is H or C₁-C₈-alkyl, preferably Rₓ is H; G is S, O or NR₁₀, wherein R₁₀ is H or C₁-C₈-alkyl, preferably R₁₀ is H; and ● is as defined in any one of the preceding items; wherein optionally a linker is arranged between ● and Q.

183. The compound according to item 182, or the conjugate of an antibody molecule according to item 185, wherein Z is

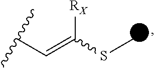

Rₓ is H or C₁-C₈-alkyl, preferably Rₓ is H, and ● is as defined in any one of the preceding items; wherein optionally a linker is arranged between ● and Q.

184. The compound according to item 180, or the conjugate of an antibody molecule according to item 180, wherein Z is

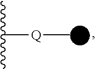

wherein [symbol] indicates the attachment point to the phosphorus and ● is as defined in any one of the preceding items; and Q is a five- or six-membered heterocyclic moiety comprising 1, 2 or 3 heteroatoms independently selected from the group consisting of N, O or S; wherein optionally a linker is arranged between ● and Q.

185. The compound according to item 184, or the conjugate of an antibody molecule according to item 184, wherein Z is selected from the group consisting of

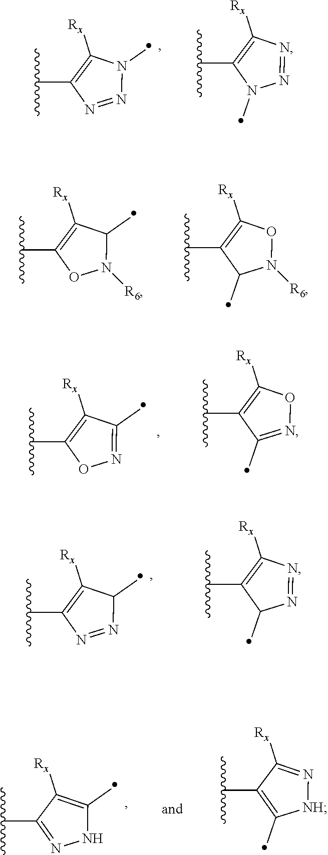

wherein Rₓ is H or C₁-C₈-alkyl, preferably Rₓ is H; R₆ is C₁-C₈-alkyl, and ● is as defined in any one of the preceding items; wherein optionally a linker is arranged between ● and Q.

186. The compound according to item 185, or the conjugate of an antibody molecule according to item 185, wherein Z is

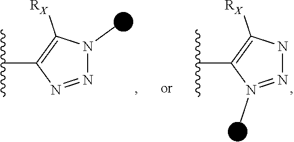

preferably Z is

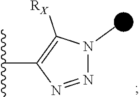

Rₓ is H or C₁-C₈-alkyl, preferably Rₓ is H, and ● is as defined in any one of the preceding items; wherein optionally a linker is arranged between ● and Q.

187. The compound according to item 180, or the conjugate of an antibody molecule according to item 180, wherein Z

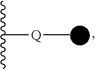

wherein [symbol] indicates the attachment point to the phosphorus and ● is as defined in any one of the preceding items; and Q is a moiety comprising a carbon-carbon triple bond bound to the phosphorus in the compound of formula (IV*) or the moiety of formula (V), and an optionally substituted phenyl group bound to the carbon-carbon triple bond, or
Q is a moiety comprising a carbon-carbon triple bond bound to the phosphorus in formula (IV*) or the moiety of formula (V), and an optionally substituted carbon-carbon double bond bound to the carbon-carbon triple bond; wherein optionally a linker is arranged between ● and Q.

188. The compound according to item 187, or the conjugate of an antibody molecule according to item 190, wherein Z is

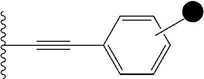

or wherein Z is

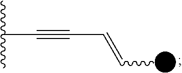

wherein ● is as defined in any one of the preceding items; wherein optionally a linker is arranged between ● and Q.

189. The compound according to any one of items 180 to 188, or the conjugate of an antibody molecule according to any one of items 180 to 188, wherein ● represents an amino acid, a peptide, a protein, an antibody, a nucleotide, an oligonucleotide, a saccharide, a polysaccharide, a radioactive or non-radioactive nuclide, biotin, a reporter enzyme, a protein tag, a fluorophore such as CY₅, fluorescein or EDANS, biotin, a linker, a drug, a linker-drug conjugate, a linker-fluorophore conjugate, a polymer, a small molecule, an optionally substituted C₁-C₈-alkyl, an optionally substituted phenyl, or an optionally substituted aromatic 5- or 6-membered heterocyclic system; wherein optionally a linker is arranged between ● and Q.

190. The compound according to item 189, or the conjugate of an antibody molecule according to item 189, wherein ● represents an amino acid, a peptide, a protein, an antibody, a nucleotide, an oligonucleotide, a saccharide, a polysaccharide, a radioactive or non-radioactive nuclide, biotin, a reporter enzyme, a polymer, an optionally substituted C₁-C₈-alkyl, an optionally substituted phenyl, or an optionally substituted aromatic 5- or 6-membered heterocyclic system; wherein optionally a linker is arranged between ● and Q.

191. The compound according to item 189 or 190, or the conjugate of an antibody molecule according to item 189 or 190, wherein ● represents an amino acid, a peptide, a protein, an antibody, a nucleotide or an oligonucleotide; wherein optionally a linker is arranged between ● and Q.

192. The compound according to item 189, or the conjugate of an antibody molecule according to item 189, wherein ● represents a drug, a protein tag, or a fluorophore such as CY₅, fluorescein or EDANS, biotin, a protein, a peptide, an antibody or an oligonucleotide; wherein optionally a linker is arranged between ● and Q.

193. The compound according to item 189, or the conjugate of an antibody molecule according to item 189, wherein ● represents a linker, a drug or a linker-drug conjugate.

194. The compound according to item 189, or the conjugate of an antibody molecule according to item 189, wherein ● represents a linker, a fluorophore, or a linker-fluorophore conjugate.

195. The compound according to item 189, or the conjugate of an antibody molecule according to item 189, wherein ● represents a small molecule, a fluorophore, a peptide, a protein, or an antibody; wherein optionally a linker is arranged between ● and Q.

196. The compound according to any one of items 180 to 188, or the conjugate of an antibody molecule according to any one of items 180 to 188, wherein ● represents a small molecule; C₁-C₈-alkyl optionally substituted with at least one of (C₁-C₈-alkoxy), wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, Br, I, —NO₂, —N(C₁-C₈-alkyl)H, —NH₂, —N₃, —N(C₁-C₈-alkyl)₂, ═O, C₃-C₈-cycloalkyl, —S—S—(C₁-C₈-alkyl), hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₂-C₈-alkynyl; wherein optionally a linker is arranged between ● and Q; or
● represents phenyl optionally independently substituted with at least one of C₁-C₈-alkyl, (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, F, Cl, I, Br, —NO₂, —N(C₁-C₈-alkyl)H, —NH₂, —N(C₁-C₈-alkyl)₂, or hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30;
wherein optionally a linker is arranged between ● and Q; or
● represents a 5- or 6-membered heteroaromatic system such as optionally substituted triazolyl or optionally substituted pyridyl; wherein optionally a linker is arranged between ● and Q.

197. The compound according to item 196, or the conjugate of an antibody molecule according to item 196, wherein ● represents a small molecule, C₁-C₈-alkyl, C₁-C₈-alkyl substituted with —S—S—(C₁-C₈-alkyl), C₁-C₈-alkyl substituted with (C₁-C₈-alkoxy), wherein n is 1,2, 3, 4, 5, 6, 7, 8, 9, 10, 11, 12, 13, 14, 15, 16, 17, 18, 19, 20, 21, 22, 23, 24, 25, 26, 27, 28, 29 or 30; hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; C₂-C₈-alkenyl; C₁-C₈-alkyl substituted with optionally substituted phenyl; or C₂-C₈-alkynyl; or phenyl; or phenyl substituted with —NO₂; or triazolyl substituted with optionally substituted C₁-C₈-alkyl; or triazolyl substituted with a fluorophore; wherein optionally a linker is arranged between ● and Q.

198. The compound according to item 196 or 197, or the conjugate of an antibody molecule according to item 196 or 197, wherein ● represents C₁-C₈-alkyl, preferably methyl, ethyl, propyl or butyl, more preferably methyl or ethyl; still more preferably ethyl; wherein optionally a linker is arranged between ● and Q.

199. The compound according to any one of items 180 to 188, or the conjugate of an antibody molecule according to any one of items 180 to 188, wherein ● is selected from the group consisting of small molecule; optionally substituted C₁-C₈-alkyl, preferably methyl, ethyl, propyl or butyl, more preferably methyl or ethyl, still more preferably ethyl; optionally substituted C₂-C₈-alkenyl; and optionally substituted C₂-C₈-alkinyl; wherein optionally a linker is arranged between ● and Q.

200. The compound according to item 199, or the conjugate of an antibody molecule according to item 199, wherein ● is selected from the group consisting of ethyl; C₁-C₈-alkyl optionally substituted with (C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, hydroxy-(C₁-C₈-alkoxy)ₙ wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; more preferably ● is

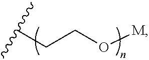

with M being hydrogen, methyl, ethyl, propyl or butyl, more preferably hydrogen or methyl, and wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 3, 4 or 5, still more preferably 4; C₁-C₈-alkyl optionally substituted with a fluorophore, more preferably ● is

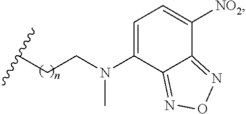

wherein n is 1, 2, 3, 4, 5, 6, 7, 8, 9 or 10, more preferably 4, 5 or 6, still more preferably 5, or more preferably ● is

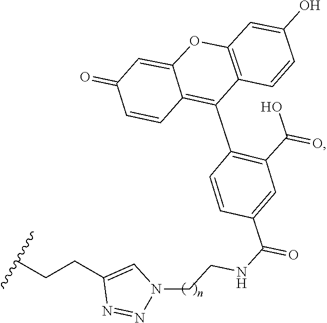

wherein n is 1, 2, 3, 4, 5, 6, 7, 8, 9 or 10, preferably 3, 4 or 5, still more preferably 4; C₂-C₈-alkynyl, preferably

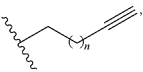

wherein n is 1, 2, 3, 4, or 5, preferably 1, 2 or 3, more preferably 1; or preferably ● is

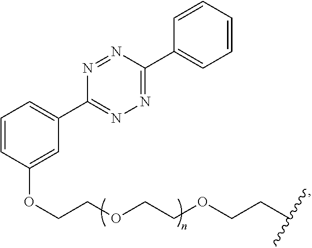

wherein n is 1, 2, 3, 4, 5, 6, 7, 8, 9, 10, 11, 12, 13, 14, 15, 16, 17, 18, 19, 20, 21, 22, 23, 24, 25, 26, 27 or 28, preferably 1, 2 or 3, more preferably 2; or preferably ● is

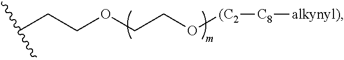

more preferably

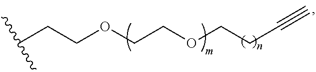

wherein m is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, more preferably 2, 3, or 4, still more preferably 3, and n is 1, 2, 3, 4 or 5, preferably 1, 2 or 3, more preferably 1; wherein optionally a linker is arranged between ● and Q.

201. The compound according to any one of items 180 to 188, or the conjugate of an antibody molecule according to any one of items 180 to 188, wherein ● is selected from the group consisting of optionally substituted aryl, preferably optionally substituted phenyl, more preferably unsubstituted phenyl; and optionally substituted heteroaryl, preferably optionally substituted triazolyl, more preferably triazolyl substituted with optionally substituted C₁-C₈-alkyl; more preferably triazolyl substituted with a fluorophore, still more preferably ● is

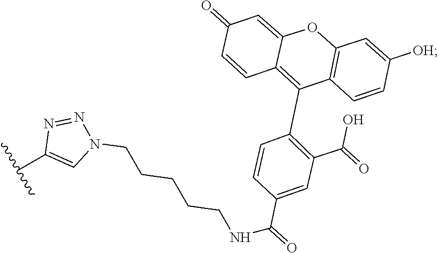 [SMILES: *c1cn(CCCCCNC(=O)c2ccc(-c3c4ccc(=O)cc-4oc4cc(O)ccc34)c(C(=O)O)c2)nn1]

or still more preferably ● is

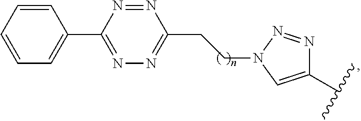

wherein n is 1, 2, 3, 4, 5, 6, 7, 8 or 9, preferably 1, 2 or 3, more preferably 1; or preferably ● is

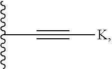

wherein K is H or C₁-C₈-alkyl, preferably K is H; wherein optionally a linker is arranged between ● and Q.

### EXAMPLES

The present invention is further illustrated by the following examples. Yet, the examples and specific embodiments described therein must not be construed as limiting the invention to such specific embodiments.

Among other things, the inventors have shown that thiol conjugates generated with diethynyl-phosphinates are highly stable in human serum and in the presence of small thiols, using a small-molecule based FRET-system. Moreover, diethynyl-phosphinates were used to site-selectively modify proteins for biological applications. Also, diethynyl phosphinates were employed in the generation of functionalized, rebridged antibodies that were shown to remain selective for their antigen.

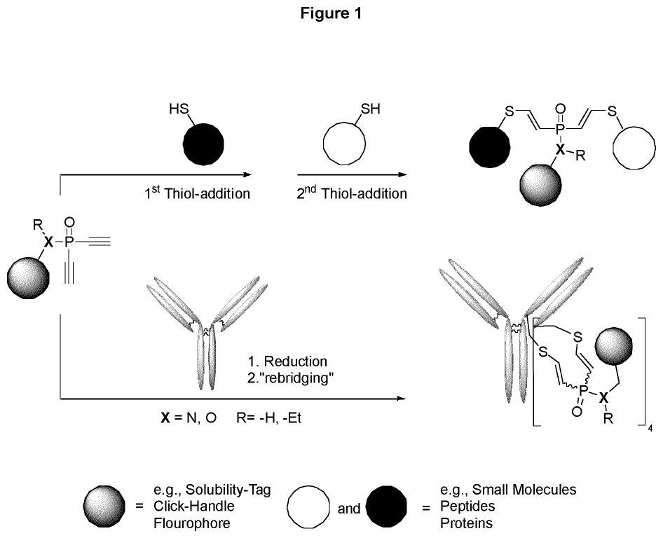

FIG. 1 shows the development of substituted diethynyl-phosphinates as reagents for selective thiol-thiol bio-conjugation and rebridging of native disulphides, e.g. in therapeutic antibodies, in accordance with the present invention.

### Example 1: Synthesis of Diethynyl-Phosphinates and Reactivity Towards Thiols

Starting from the commercially available ethyl dichlorophosphite and ethynylmagnesium bromide, the inventors obtained ethyl diethynyl phosphinite (I), Scheme 1a. Phosphinite I was successfully oxidized with hydrogen peroxide ethyl diethynyl phosphinate (1) in 72% yield. To test its reactivity towards thiols, compound 1 was mixed with an excess of ethanethiol (3 eq.) in DMF. Indeed, full conversion of the starting material into the double thiol-adduct was observed after reacting it for 5 minutes at room temperature (r.t., Scheme 1a).

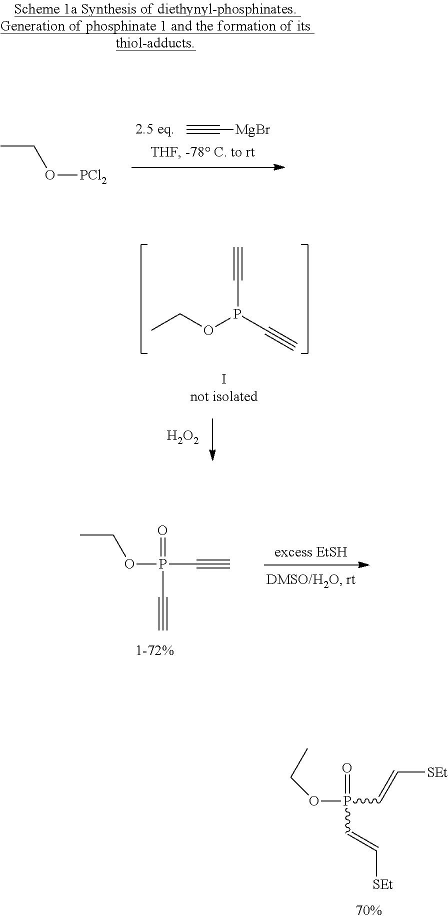

### Scheme 1a: Synthesis of Diethynyl-Phosphinates. Generation of Phosphinate 1 and the Formation of its Thiol-Adducts

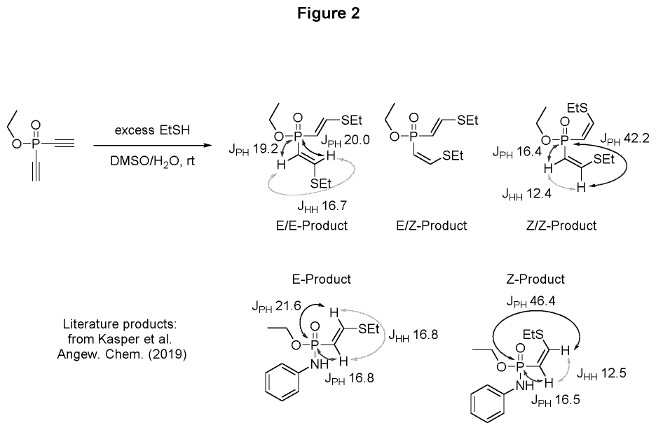

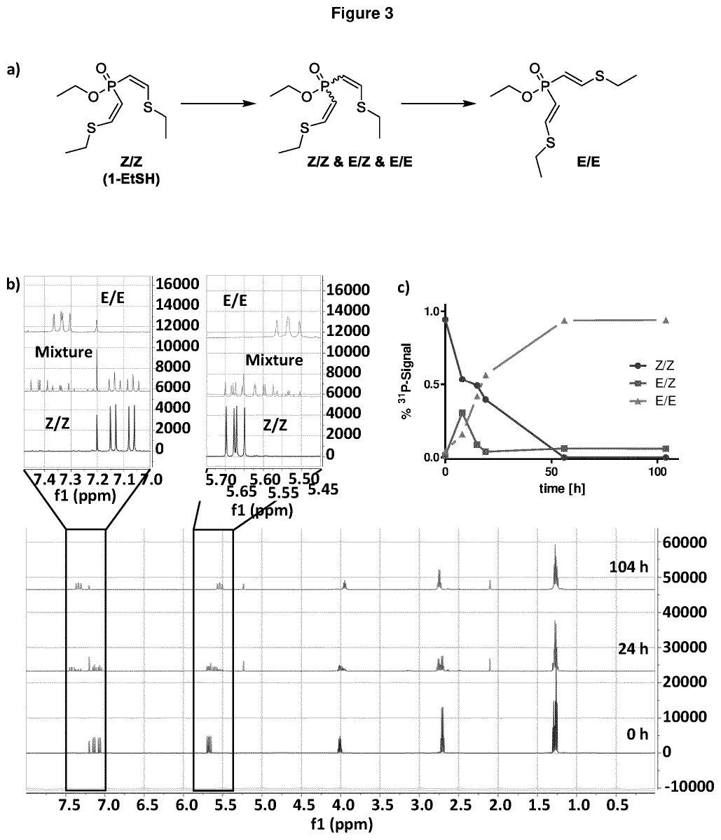

Interestingly, a mixture of three different isomers was formed (as determined by UPLC) that could be separated by means of semi-preparative HPLC (70% combined isolated yield). Characterisation of the obtained compounds with nuclear magnetic resonance (NMR) spectroscopy, revealed that the observed ratio of 45:50:5 corresponds to the Z/Z-, E/Z-, and E/E-isomer respectively. (FIG. 2) However, slow isomerization of the Z/Z- and E/Z isomer to form the E/E-product was observed. (FIG. 3).

FIG. 2 shows the E/Z-selectivity of the thiol addition with diethynyl-phosphinates. NMR analysis of the isolated compounds allowed to identify the different isomers based on the characteristic coupling constants of the alkene-protons and their comparison to previously characterized thioladducts (Kasper, M.; Glanz, M.; Stengl, A.; Penkert, M.; Klenk, S.; Sauer, T.; Schumacher, D.; Helma, J.; Krause, E.; Cardoso, M. C.; Leonhardt, H.; Hackenberger, C. P. R. Cysteine-Selective Phosphonamidate Electrophiles for Modular Protein Bioconjugations. Angew. Chemie Int. Ed. 2019, 58 (34), 11625-11630. https://doi.org/10.1002/anie.201814715; Mikolajczyk, M.; Costisella, B.; Grzejszczak, S. Organosulphur Compounds-XXIX. Synthesis and Pummerer Rearrangement of β-Phosphoryl Sulphoxides. Tetrahedron 1983, 39 (7), 1189-1193. https://doi.org/10.1016/S0040-4020(01)91883-6).

FIG. 3 shows isomerization of the formed thiol-adduct from Z/Z to E/E The isolated Z/Z-product (1-EtSH) was placed into an NMR-tube and dissolved in CDCl₃. ¹H- and ³¹P NMR were recorded over a period of four days to monitor the isomerization into the E/E-form. FIG. 3a shows a schematic representation of the isomerization process. FIG. 3b shows ¹H-NMR scans illustrating the stepwise isomerization. FIG. 3c shows Integrals of the ³¹P-NMR signals corresponding to the different isomers over time.

Experimental procedures are given in Example 18 below.

### Example 2: Synthesis of Diethynyl-Phosphinates with Functional O-Substituents

In order to access compounds with functional O-substituents a one-pot two-step reaction starting from diethyl-phosphoramidous dichloride was developed. (Scheme 1b). Accordingly, in an initial step, diethyl phosphoramidous dichloride was substituted two times with ethynyl magnesium bromide. Using this route diethynyl phosphinates bearing alkynes as click-handle (2), as well as tetraethylenglycol (3) and the fluorophore NBD (4) were synthesized (Scheme 1b). In the course of this synthesis, diethynyl phosphinates were found to be heat-sensitive and moderately stable on silica after they are oxidized. For this reason, the inventors purified the compound as P(III)-derivative and oxidized as final step. Further, the intermediate obtained by addition of the ethynyl magnesium bromide to diethyl-phosphoramidous dichloride, could be oxidized using hydrogen peroxide to give II.

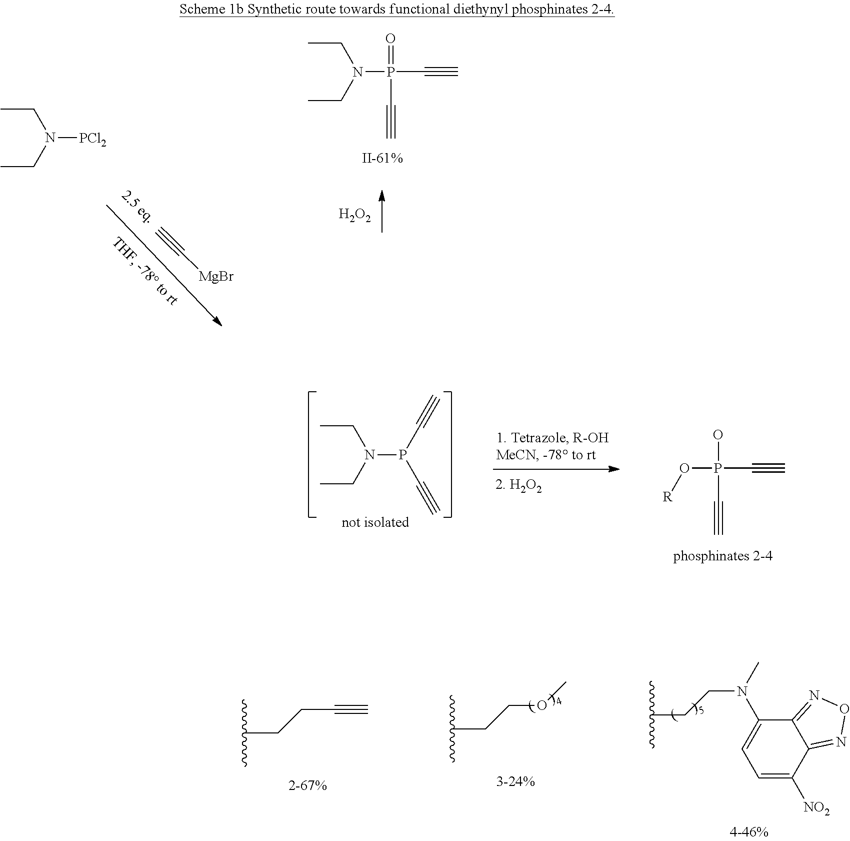

### Scheme 1b: Synthetic Route Towards Functional Diethynyl Phosphinates 2-4

Experimental procedures are given in Example 18 below.

### Example 3: Stability of Protein-Conjugates

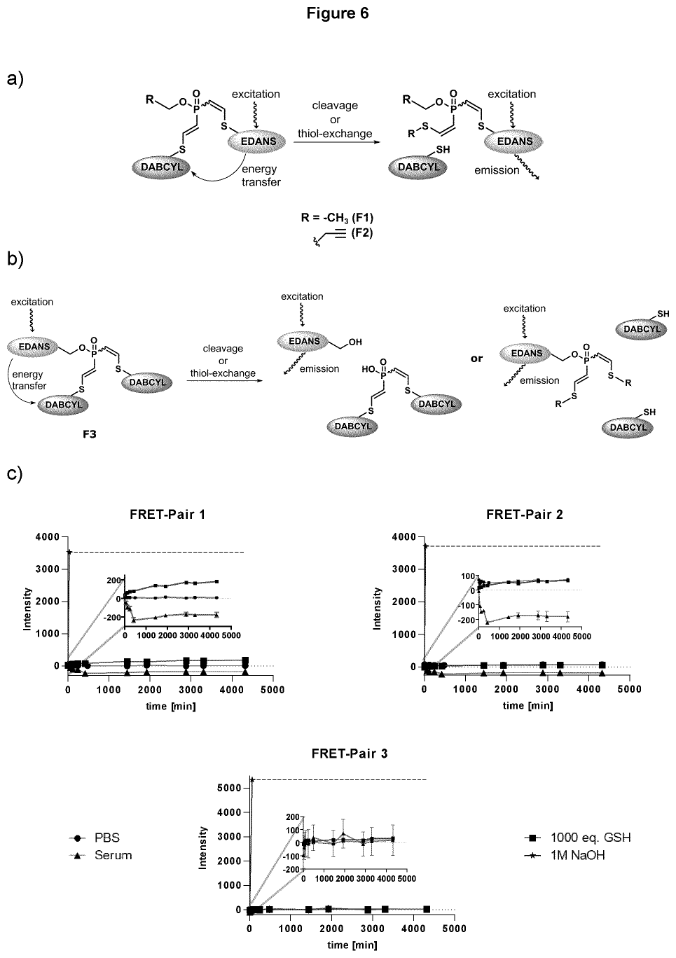

Stability of protein-conjugates under physiological and biologically relevant conditions (e.g. in the presence of other thiols) is of utmost importance for their successful application. Especially, maleimide- and electron-deficient alkyne-based thiol adducts have been reported to be susceptible towards exchange with other thiols as they are present inside of cells or in blood serum (S. B. Gunnoo, A. Madder, ChemBioChem 2016, 17, 529-553; H.-Y. Shiu, T.-C. Chan, C.-M. Ho, Y. Liu, M.-K. Wong, C.-M. Che, Chem.-A Eur. J. 2009, 15, 3839-3850). To investigate the stability of phosphonamidate and phosphonothiolate based thiol-adducts, a fluorescence-quenching-assay has been employed previously (A. L. Baumann, S. Schwagerus, K. Broi, K. Kemnitz-Hassanin, C. E. Stieger, N. Trieloff, P. Schmieder, C. P. R. Hackenberger, J. Am. Chem. Soc. 2020, 142, 9544-9552; M. Kasper, M. Glanz, A. Stengl, M. Penkert, S. Klenk, T. Sauer, D. Schumacher, J. Helma, E. Krause, M. C. Cardoso, H. Leonhardt, C. P. R. Hackenberger, Angew. Chemie Int. Ed. 2019, 58, 11625-11630). Using this assay, the stability of thiol-conjugates generated from diethynyl-phosphinates 1, as well as the stability of the P—O bond after thiol conjugation was investigated. Quenched products F1-F3 were synthesized from peptide P3, EDANS-SH or EDANS-N₃ and the corresponding phosphinates 1 or 3 were used as bireactive ethyni-phosphinates. (Scheme 1c and FIG. 4) For all constructs, excellent stability in physiological buffer, human serum and in the presence of excess free thiols was observed over the course of several days. (FIG. 5a-c and FIG. 6). Only under strong basic conditions, the conjugates degrade via beta-elimination at the linked Cys residue.

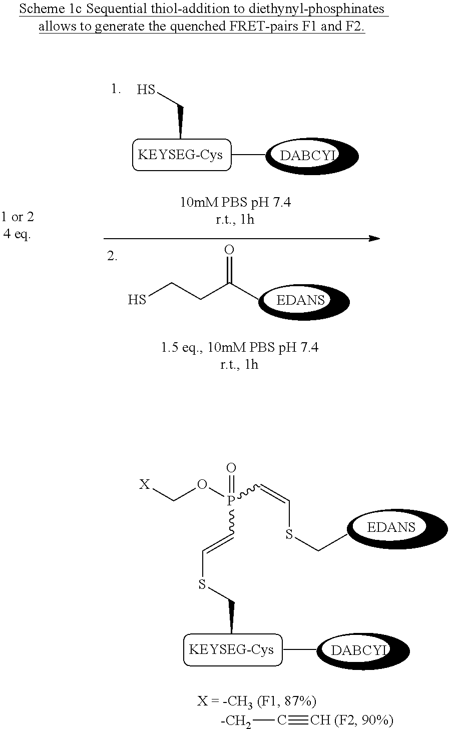

### Scheme 1c: Sequential Thiol-Addition to Diethynyl-Phosphinates Allows to Generate the Quenched FRET-Pairs F1 and F2.

FIG. 4 shows the synthesis of quenched FRET-pairs F1-F3. Synthetic procedure for the generation of quenched fluorophore pairs: Quenched FRET-Pair 1 was synthesized from peptide 2 and excess (10 eq.) phosphinate 1 in PBS (pH 7.4). After purification of the intermediate, it was reacted with 1.2 eq. EDANS-thiol in PBS. F1 was purified via semi-preparative HPLC (1.67 mg, 86%). Quenched FRET-Pair 2 was synthesized analogously to F1, only phosphinate 2 was used as a linker. F2 was purified via semi-preperaive HPLC (1.94 mg, 91%). Quenched FRET-Pair 3 was synthesized from 2 eq. peptide 2 and phosphinate 2 (1 eq.) in PBS. Subsequently, EDANS-azide was conjugated to the phosphinate side-chain using CuBr (10 mol %) as catalyst. F3 was purified via semi-preperaive HPLC (2.13 mg, 78%).

FIG. 5a shows a FRET-quenching assay to investigate the stability of the thiol-conjugates. FIG. 5b and FIG. 5c show the observed EDANS-fluorescence for constructs F1 & F2 in PBS, PBS supplemented with glutathione, human serum and in 0.1M aq. NaOH over 72 h.

FIG. 6 shows the stability testing of diethynyl-phosphinate conjugates using a fluorophore-quencher based assay to investigate the stability of phosphinate-thiol adducts. FIG. 6a and FIG. 6b show the structure of the phosphinate linked dye-quencher conjugates and principles of the fluorescence-quencher based readout. FIG. 6c shows fluorescence measurements for conjugates F1-F3. Stability studies of the Dabcyl-EDANS adducts were conducted in 96-well plate (Corning 3615, black with clear, flat bottom) at least in triplicates. 5 μl of a 200 μM Stock solution of the Dabcyl-EDANS conjugates and 95 μl of the respective test solutions were added to each well. Human serum was purchased from Sigma Aldrich. Glutathione was dissolved at a concentration of 10 mM in PBS and the pH was adjusted to 7.4. 0.1 mM NaOH studies were conducted at 200 μM, neutralized to pH 7 and diluted to 10 μM before fluorescence measurements. Fluorescence was measured on a Tecan Safire plate reader. Excitation: 360 nm, emission: 508 nm, bandwidth: 5 nm at 20° C.

Experimental procedures are given in Examples 17 and 18 below.

### Example 4: Protein Modification

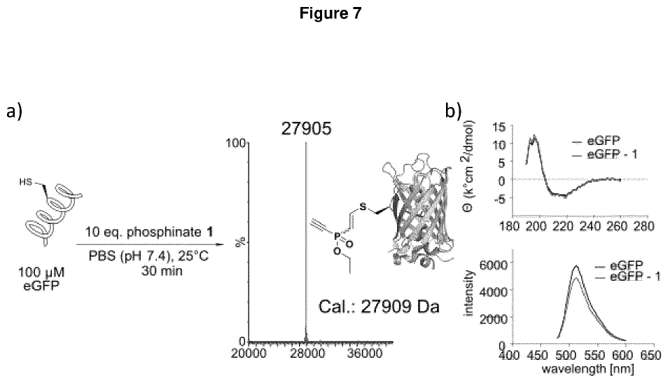

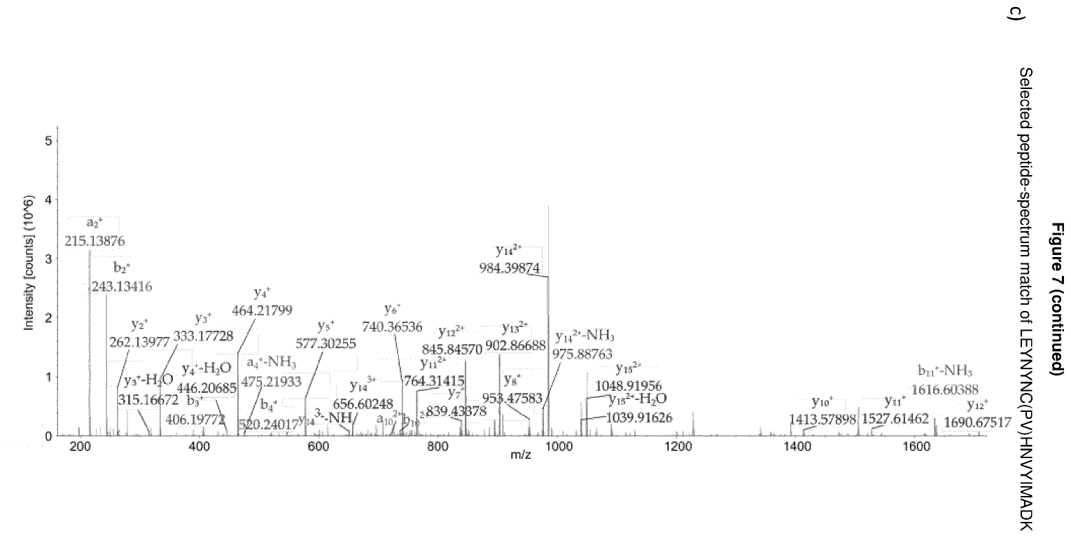

Having demonstrated that this compound-class is highly thiol-reactive and that the resulting conjugates are stable under various biologically relevant conditions, their applicability for protein modifications was tested. At first phosphinate-1 was reacted with an eGFP mutant containing a single addressable Cys (eGFP C70M S147C). Using 10 eq. phosphinate in PBS pH 7.4 containing 10% DMSO as a co-solvent, the reaction reached completion after 30 minutes (FIG. 5d). Analysis of the protein conjugate via CD- and fluorescence-spectroscopy showed that the secondary structure of eGFP is not altered upon modification (FIG. 7). Tandem-mass spectrometry (MS/MS) analysis of the tryptically-digested protein verified that no amino acid other than Cys was modified by the phosphinate (FIG. 7).

To prove the general applicability of Cys specific protein labeling using diethynyl phosphinates, various proteins of different size and nature (Ubiquitin G76C, Histone H4, recombinant human albumin and NLS-mCherry) bearing one single addressable Cys residue were labeled. Incubation of the proteins (10-50 μM in PBS pH 7.4) with 10 eq. phosphinate resulted in single-modified proteins after 10-60 minutes of reaction time. (FIG. 5d) In contrast, reaction of 1 with an eGFP variant containing no addressable Cys (eGFP C70M) did not result in any protein labeling, further supporting excellent Cys selectivity.

FIG. 5d shows a General scheme for the site-selective protein-modification using diethynyl-phosphinates and deconvoluted intact-protein-MS spectra of successfully labelled proteins.

FIG. 7 shows eGFP-labeling with phosphinate 1 via determination of the effect of labeling proteins with Diethynyl phosphinates on the secondary structure. FIG. 7a shows labeling of eGFP (C70M S147C) with 10 eq. 1 in PBS (30 min, r.t.) followed by purification via 0.5 mL Zeba™ Spin Desalting Columns with 7K MWCO (Thermo Fisher Scientific, USA) into phosphate-buffer (20 mM, pH 7.5). Complete labeling was verified via intact-protein MS. FIG. 7b shows CD and fluorescence spectra recorded after the protein was diluted to a concentration of 5 μM. Comparison to spectra obtained from non-modified eGFP showed no significant differences. This indicates, that the secondary structure of the protein is not affected upon labeling. FIG. 7c shows a tandem mass spectrometry analysis of in-gel trypsin-digested, labeled eGFP verified that only cysteine is labeled.

Experimental procedures are given in Examples 17 and 18 below.

### Example 5: Reaction Kinetics of Thiol Addition

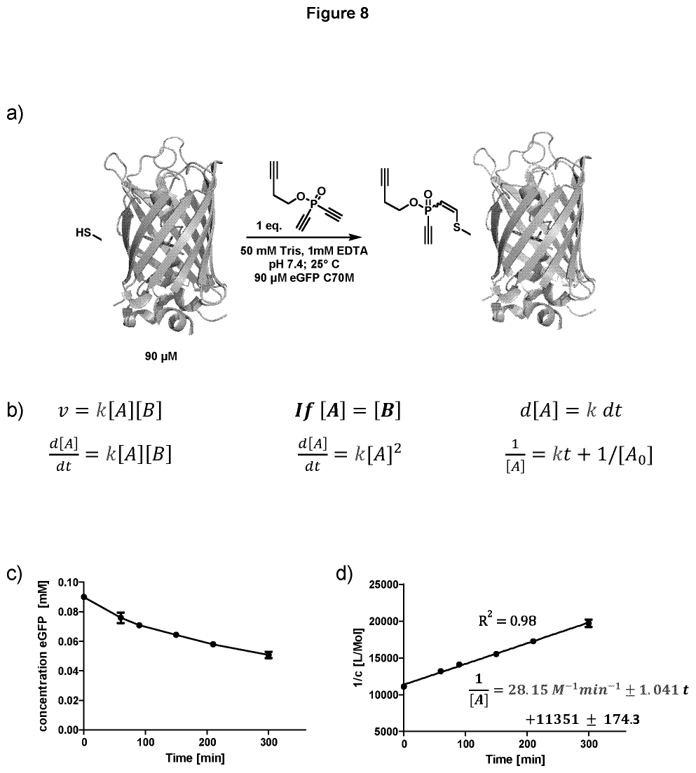

Subsequently, this new compound-class was compared with other Cys-reactive electrophiles in terms of reaction kinetics. The estimation of reaction-kinetics is particularly challenging for bis-reactive reagents because the second thiol addition interferes with the analysis. However, one observed that on protein-level the second thiol-addition (i.e. the protein-protein cross-linking) is significantly slower, probably because diffusion becomes a limiting factor. Consequently, 2 was reacted with one equivalent GFP in Tris-buffer (pH 8.3) at a concentration of 90 μM at room-temperature. The reaction progress was monitored by intact protein-MS using eGFP C70M as internal standard and revealed a second order rate constant of approximately 0.47 M⁻¹sec⁻¹ which is comparable with previously reported P(V)-electrophiles (A. L. Baumann, S. Schwagerus, K. Broi, K. Kemnitz-Hassanin, C. E. Stieger, N. Trieloff, P. Schmieder, C. P. R. Hackenberger, J. Am. Chem. Soc. 2020, 142, 9544-9552; M. Kasper, M. Glanz, A. Stengl, M. Penkert, S. Klenk, T. Sauer, D. Schumacher, J. Helma, E. Krause, M. C. Cardoso, H. Leonhardt, C. P. R. Hackenberger, Angew. Chemie Int. Ed. 2019, 58, 11625-11630) (FIG. 8).

FIG. 8 shows estimation of the reaction kinetics of diethynyl phosphinates with proteins. Determination of the second-order rate constant of the reaction between eGFP (C70M S147C) and phosphinate 2. FIG. 8a shows the Reaction conditions. Reactions were carried out in triplicate. 90 μl eGFP (0.1 mM) were mixed with 10 μl of a 0.9 mM solution of phosphinate 2 in DMSO. Samples were drawn after 60, 90, 150, 210 and 300 minutes and analyzed via intact protein-MS. FIG. 8b shows the mathematic consideration for the determination of a second order rate constant with equal concentrations of the two reactants. FIG. 8c shows the concentration of eGFP over time. Calculated by the intensity of the deconvoluted mass in relation to the internal standard eGFP (C70M). FIG. 8d shows 1/c over time. The slope corresponds to the second order rate constant. Shown are mean and error of three independent measurements.

### Example 6: Preparation of Protein Conjugates

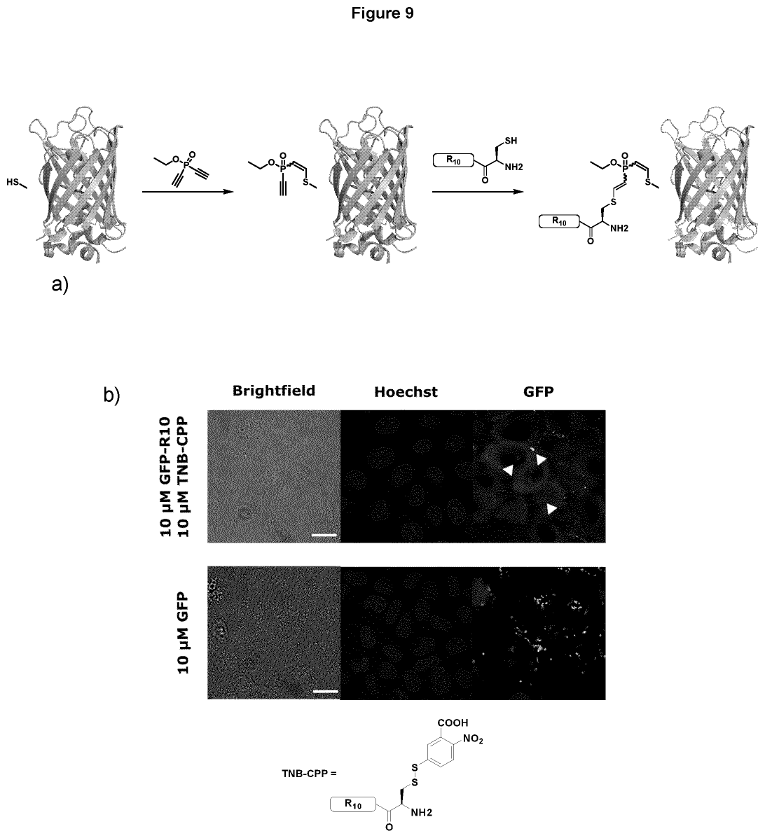

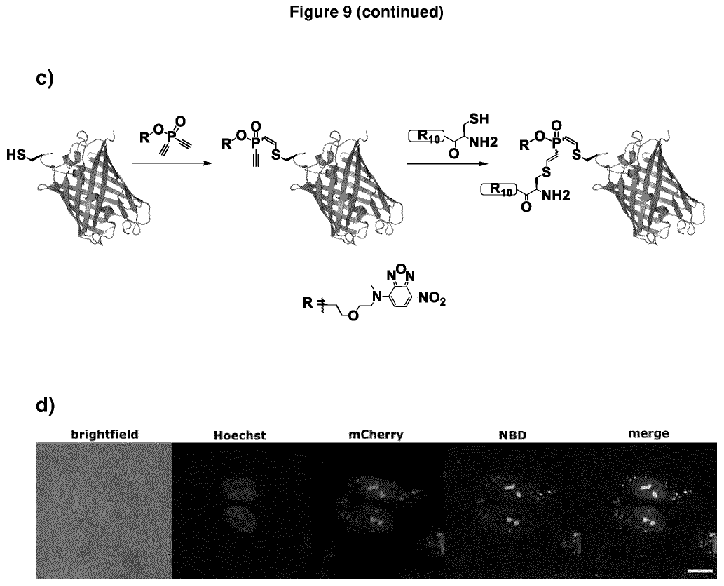

Next, a two-step labeling procedure was used to generate functional protein-conjugates. Recently, the use of linear poly-arginine peptides (R₁₀) for non-endosomal cellular delivery of proteins into living cells with immediate bioavailability has been described (C. P. R. Schneider, Anselm F. L.; Kithil, Marina; Cardoso, M. Cristina; Lehmann, Martin; Hackenberger, 2021, submitted Manuscript). Following the two-step labeling procedure, a Cys containing R₁₀ was conjugated to eGFP via phosphinate 1. (FIG. 9a) With the eGFP-R₁₀ conjugate in hand, the cellular uptake into live HeLa-Kyoto cells was probed following a previously developed protocol (C. P. R. Schneider, Anselm F. L.; Kithil, Marina; Cardoso, M. Cristina; Lehmann, Martin; Hackenberger, 2021, submitted Manuscript). Live-cell imaging showed cytosolic localization of the protein. Moreover, localization in the nucleoli was observed, which indicates that the phosphinate-linkage is stable inside of living cells (FIG. 5e and FIG. 9b). Also, the Cys-containing R₁₀ was conjugated to NLS-mCherry-5 via NBD phosphinate 5 (FIG. 5d and FIG. 9c).

FIG. 5e shows that conjugation of a cell penetrating R₁₀-peptide to mCherry-5 allows delivery of mCherry into living cells with nucleolar localization and co-localization of mCherry with NBD. This also indicates that the phosphinate linkage is stable inside of living cells (FIG. 5e and FIG. 9d).

FIG. 9 shows diethynyl phosphinates as linker-molecules for the attachment of cell-penetrating peptides to proteins. FIG. 9a shows a schematic illustration of the generation of the eGFP-R₁₀-conjugate. FIG. 9b shows Fluoresence imaging of HeLa-cells after incubation with eGFP alone and eGFP-R₁₀ following the procedure of Schneider et al. (Schneider, Anselm F. L.; Kithil, Marina; Cardoso, M. Cristina; Lehmann, Martin; Hackenberger, Christian P. R.; Cellular uptake of Large Biomolecules Enabled by Cell-surface-reactive Cell-penetrating Peptide Additives, Nat. Chem. (2021) accepted Manuscript https://doi.org/10.1038/s41557-021-00661-x.) HeLa Kyoto cells were grown at 37° C. in a humidified atmosphere with 5% CO₂ in DMEM 4.5 g/L glucose with 10% fetal bovine serum (FBS). 15′000 HeLa Kyoto cells were seeded per well of a 8-well ibidi μ-slide. Cells were left to adhere and grow for 24 hours at 37° C. and 5% CO₂. Cells were washed twice with Fluorobrite DMEM without FBS and incubated with 10 μM eGFP-1-R₁₀ and additives (10 μM TNB-R₁₀) in Fluorobrite DMEM without FBS. Cells were incubated for 1 h at 37° C. and 5% CO₂. Cells were washed three times with Fluorobrite DMEM with 20 mM glutamine and 10% FBS. The cells were then covered with Fluorobrite DMEM with 20 mM glutamine and 10% FBS containing Hoechst stain (Hoechst 33342). Live cell microscopy images of the eGFP uptake experiments were acquired on Nikon-CSU spinning disc microscope with a CSU-X1 (Andor) and live cell incubation chamber (OKOlab). All live cell images were acquired using a PlanApo 60× NA 1.4 oil objective (Nikon) and an EMCCD (AU888, Andor). Brightfield images were acquired along with fluorescence images. Standard laser, a quad Dicroic (400-410, 486-491, 560-570, 633-647, AHF) and Emission filters were used for the acquisition of confocal fluorescence images (BFP (Hoechst 33342) ex.: 405 nm em.: 450/50, GFP (GFP), ex: 488 em: 525/50. Images show brightfield in grey, eGFP channel in green and Hoechst 33342 in blue. Scale bar represents 20 μm.

Diethynyl phosphinates as linker-molecules for the generation of cell-penetrating protein-double conjugates: To further validate the integrity of the whole phosphinate-linkage, the inventors used phosphinate 5 with NBD at the O-substituent to generate an mCherry-NBD-R₁₀ double-conjugate. This conjugate was also delivered into living cells, and co-localization of mCherry with NBD was observed. This further points towards the intracellular stability of the whole phosphinate-linkage in the cargo-conjugate.

FIG. 9c shows a schematic illustration of the generation of the mCherry-NBD-R₁₀-conjugate. FIG. 9d shows fluorescence imaging of CCL2-cells after incubation with the mCherry double-conjugate following the procedure published by Schneider et al (Schneider, Anselm F. L.; Kithil, Marina; Cardoso, M. Cristina; Lehmann, Martin; Hackenberger, Christian P. R.; Cellular uptake of Large Biomolecules Enabled by Cell-surface-reactive Cell-penetrating Peptide Additives, Nat. Chem. (2021) accepted Manuscript. https://doi.org/10.1038/s41557-021-00661-x.).

Images show brightfield in grey, eGFP channel (NBD) in green, mCherry in red and Hoechst 33342 in blue. Scale bar represents 20 μm

Experimental procedures are given in Examples 17 and 18 below.

### Example 7: Rebridging of Disulfides in Antibody

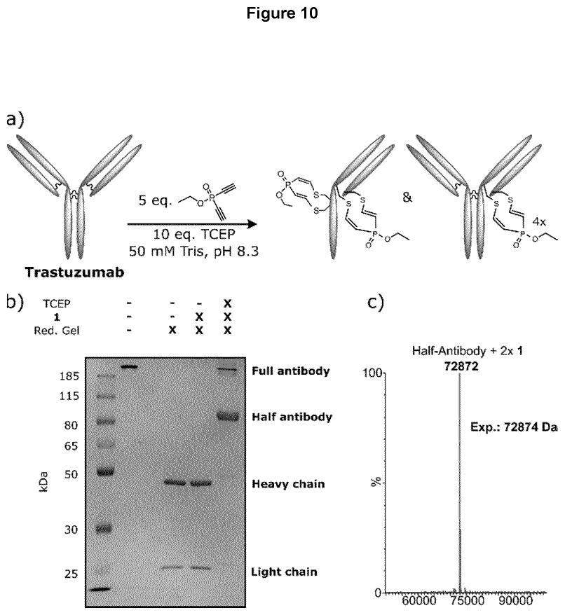

Motivated by these results, the inventors aimed for the rebridging of disulfides in IgG antibodies. Diethynyl phosphinates offer the potential to modify all four interchain disulfides, leading to a precise antibody-to-cargo ratio of four. Using the Her2-targeting monoclonal IgG antibody Trastuzumab, the ability of diethynyl phosphinates for antibody rebridging was investigated (FIG. 10).

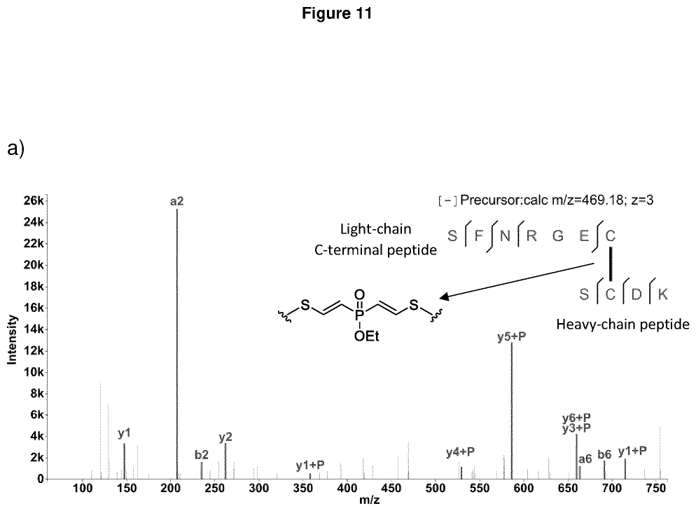

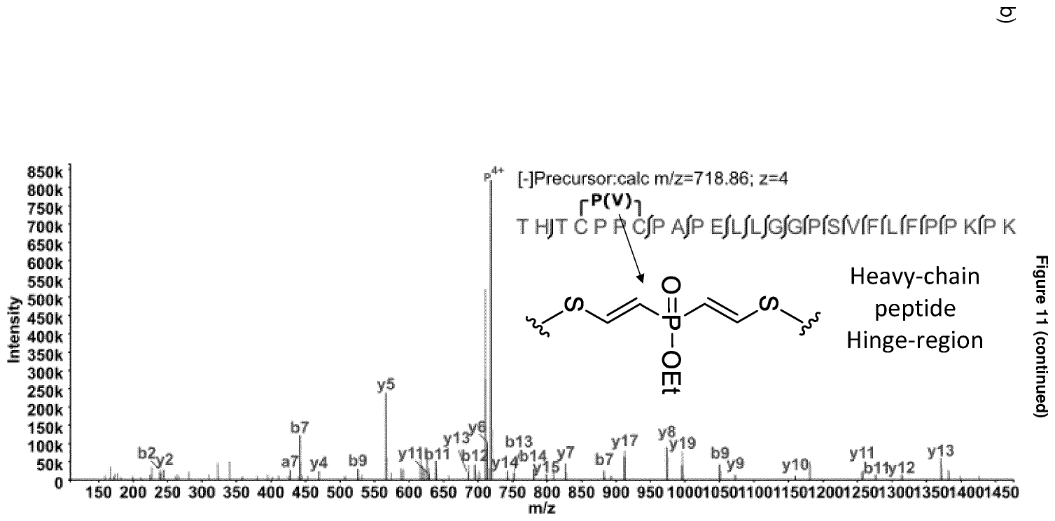

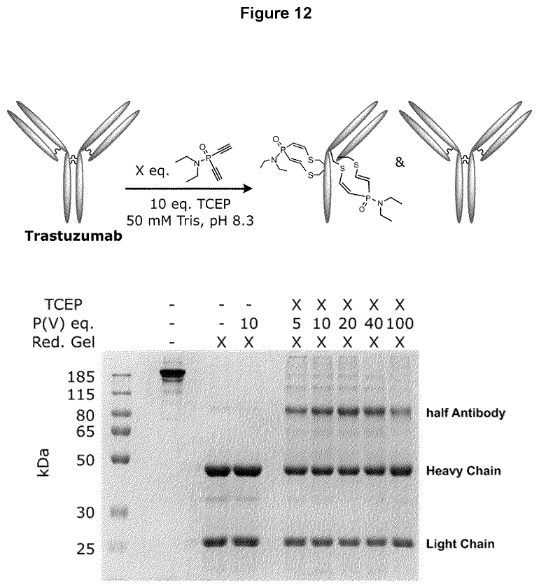

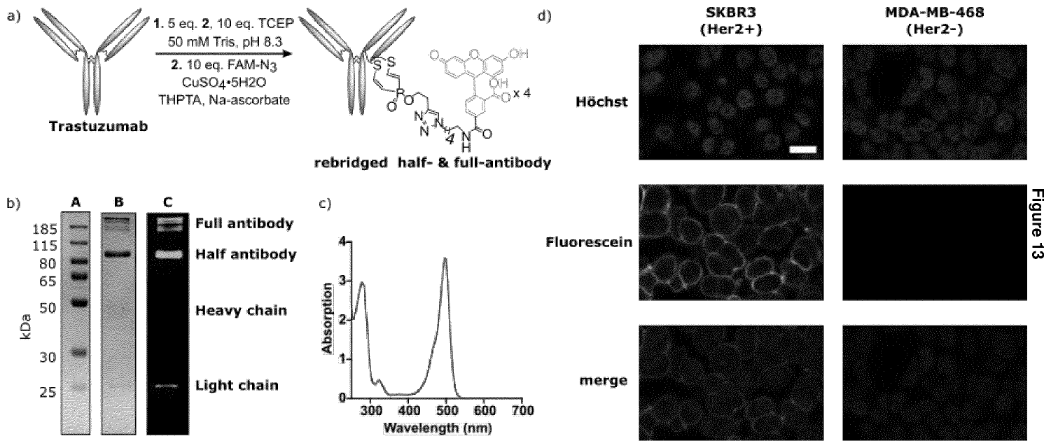

Therefore, a previously described protocol for antibody modification was slightly adopted (S. J. Walsh, S. Omarjee, W. R. J. D. Galloway, T. T.-L. Kwan, H. F. Sore, J. S. Parker, M. Hyvönen, J. S. Carroll, D. R. Spring, Chem. Sci. 2019, 10, 694-700). At first Trastuzumab (5 mg/ml) was reduced using 10 eq. (2.2 eq. per disulfide) TCEP at 37° C. for half an hour. Subsequently, 5 eq. phosphinate 1 were added to the solution and the reaction was allowed to proceed at room temperature over-night. SDS-page and intact protein-MS analysis showed >95% rebridging of the antibody. The reaction yielded a mixture of the rebridged intact full antibody, and the half antibody (covalently linked heavy- & light chain, FIG. 10b and FIG. 10c), with the half antibody being the main product. In contrast, when non-reduced Trastuzumab was reacted with 1, no rebridging or modification could be observed. Using a dedicated cross-linking mass spectrometry search engine (Z. L. Chen, J. M. Meng, Y. Cao, J. L. Yin, R. Q. Fang, S. B. Fan, C. Liu, W. F. Zeng, Y. H. Ding, D. Tan, L. Wu, W. J. Zhou, H. Chi, R. X. Sun, M. Q. Dong, S. M. He, Nat. Commun. 2019, 10, DOI 10.1038/s41467-019-11337-z), the inventors were even able to validate the inter-chain cross-link formed between the heavy- and light-chain of Trastuzumab (FIG. 11). Further, phosphinamidate II was tested for antibody rebridging. SDS-gel analysis showed that II was able to cross-link the antibodies light- & heavy-chain to approximately 33% (FIG. 12).

FIG. 10 shows the reaction of Trastuzumab with diethynyl-phosphinate 1 and subsequent analysis. FIG. 10a shows the general procedure for antibody-rebridging using compound 1. FIG. 10b shows the analysis of Trastuzumab before and after the reaction via SDS-PAGE. FIG. 10c shows the deconvoluted intact protein-MS of the rebridged half-antibody (2× modified with 1) after deglycosylation by PNGaseF.

FIG. 11 shows identification of the two cross-linked cysteine residues of an antibody via cross-linking mass spectrometry. For the MS/MS analysis, rebridged Trastuzumab was deglycosylated using PNGase F followed by tryptic in-gel digest. For the identification of the rebridged cysteine residues a dedicated cross-link search engine (pLink 2, Chen, Z. L.; Meng, J. M.; Cao, Y.; Yin, J. L.; Fang, R. Q.; Fan, S. B.; Liu, C.; Zeng, W. F.; Ding, Y. H.; Tan, D.; Wu, L.; Zhou, W. J.; Chi, H.; Sun, R. X.; Dong, M. Q.; He, S. M. A High-Speed Search Engine PLink 2 with Systematic Evaluation for Proteome-Scale Identification of Cross-Linked Peptides. Nat. Commun. 2019, 10 (1). https://doi.org/10.1038/s41467-019-11337-z) was used. FIG. 11a shows the only cross-link that could be identified was the cross-link between Trastuzumab light-chain and heavy-chain cysteine. Likely, the rebridged hinge-region could not be detected, because the resulting peptide is relatively large and hydrophobic. FIG. 11b shows that, moreover, the intra-chain cross-link between the two hinge-region cysteins of the heavy-chain was identified.

FIG. 12 shows antibody rebridging using phosphinamidate II. Trastuzumab was rebridged according to the general antibody re-bridging protocol using increasing concentrations of II (see “General procedure for antibody rebridging using phosphinates”). Cross-linking of the antibodies heavy- and light-chain was analysed by SDS-PAGE.

Encouraged by the excellent rebridging efficiency and site-selectivity of diethynyl phosphinates, Trastuzumab was reacted with phosphinate 2 to allow for further functionalization of the antibody-conjugate. (FIG. 13) Following the same procedure as described for 1, complete rebridging was observed after over-night reaction. The half antibody was formed as main product. (FIG. 13b) In the next step, the antibody was conjugated to fluorescein-azide (FAM-N₃) via copper-aided azide alkyne cycloaddition (CuAAC) to generate an antibody-fluorophore conjugate (AFC). In-gel fluorescence imaging and UV-vis spectroscopy validated successful modification of the antibody. (FIG. 13b and FIG. 13c) The fluorophore to antibody ratio was calculated to be 4.03, indicating quantitative conversion. (section “Cu-click modification of Trastuzumab-2 with Fluorescein-N₃” of experimental procedures below). The functionalized Trastuzumab clearly stained outer-membrane bound Her2 on BT474 cells, while no fluorescent signal could be observed with the Her2−cell line. (FIG. 13d) This indicates that the rebridging and labeling strategy does not impede the antibodies performance.

FIG. 13 shows functional modification of Trastuzumab and its biological evaluation. FIG. 13a shows two step modification of the antibody with phosphinate 2, followed by on antibody CuAAC forming the fluorescein conjugate. FIG. 13b shows the analysis of the conjugate via SDS-PAGE using coomassie staining and in-gel fluorescence. FIG. 13c shows a UV-Vis spectrum of the fluorescein conjugated antibody. FIG. 13d shows cell-membrane labelling of Her2-positive cells without any observed staining of Her2-negative cells (Scale bar 20 μm).

Experimental procedures are given in Examples 17 and 18 below.

### Example 8: Antibody Labeling Using Thiovinyl- and Triazole-Based Ethynyl-Phosphinates

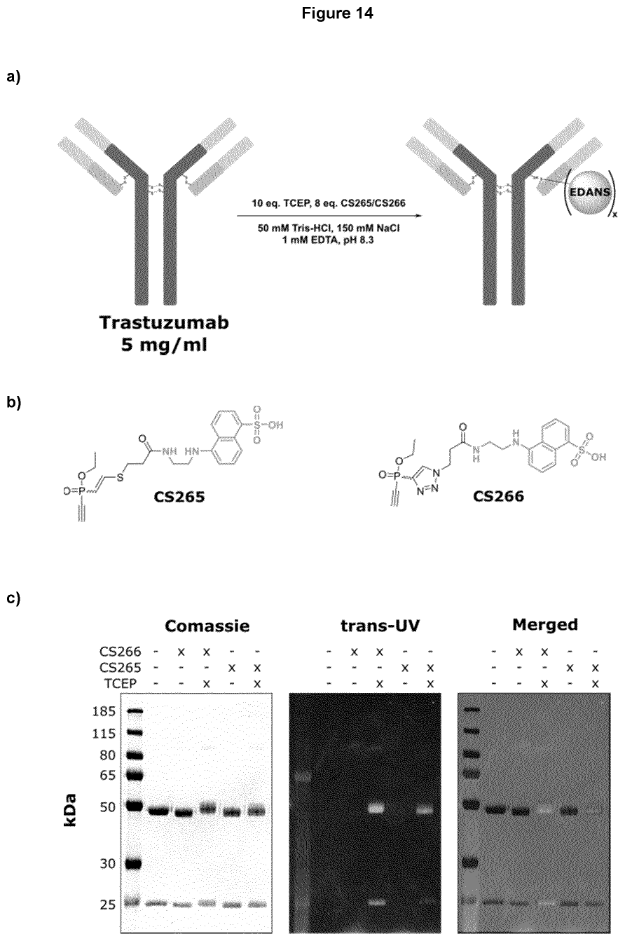

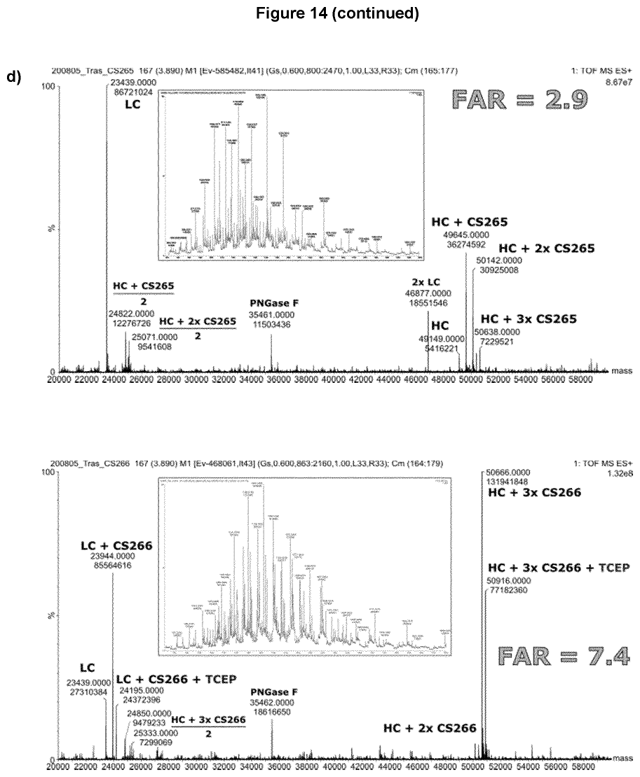

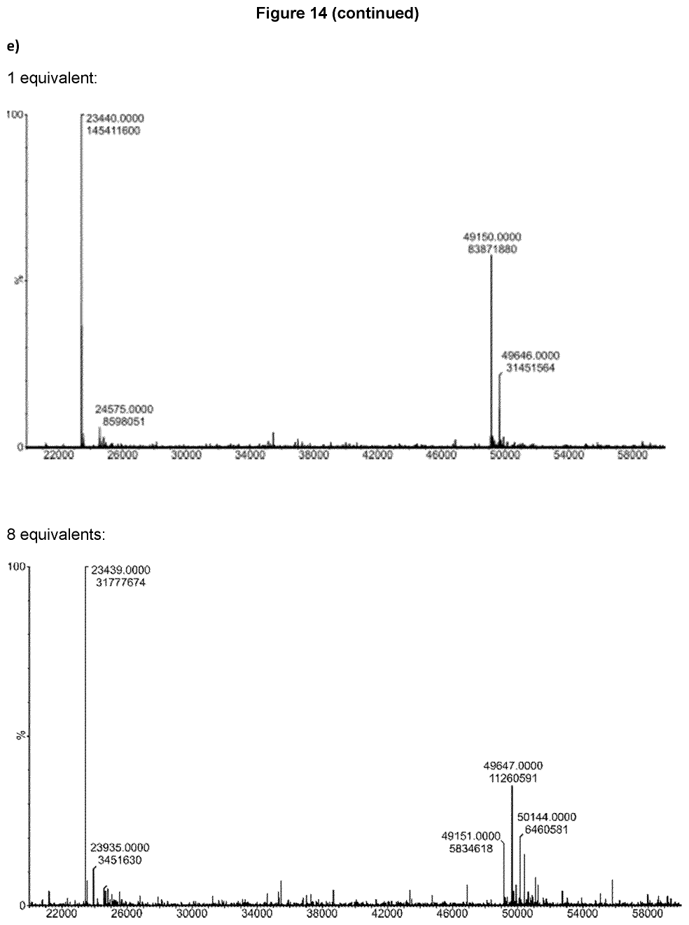

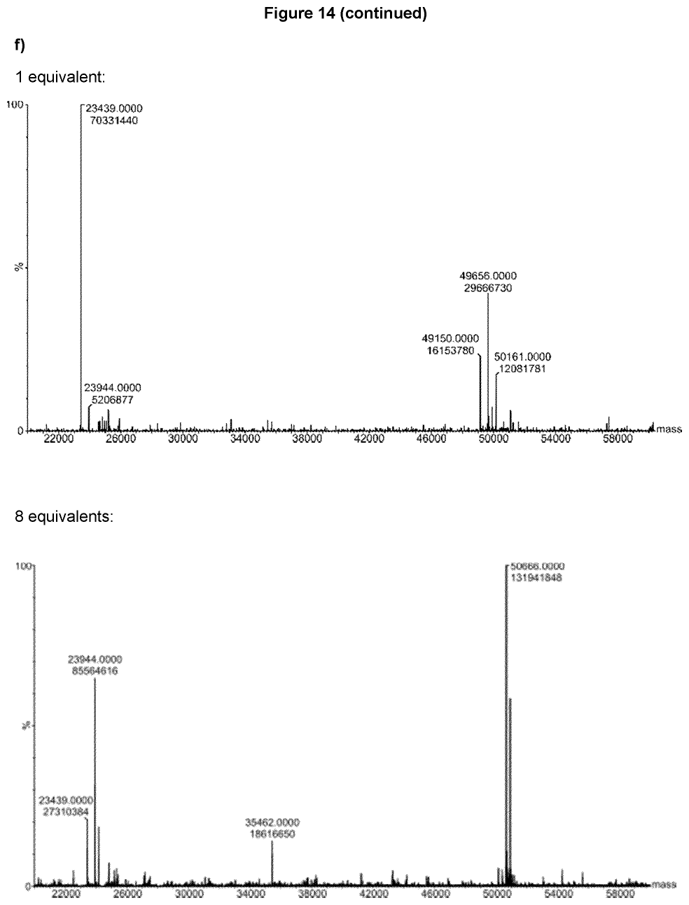

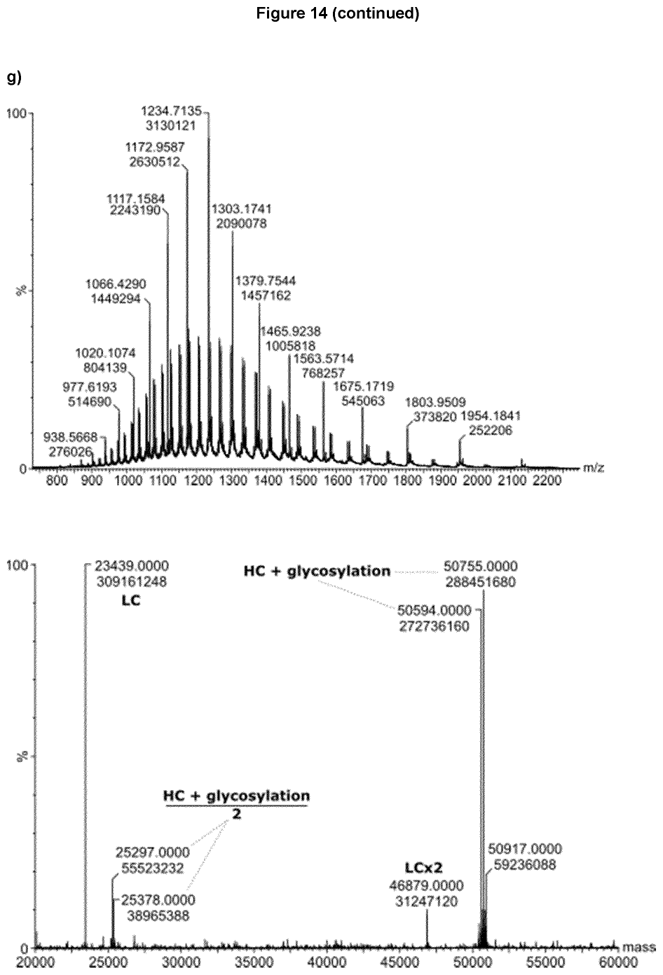

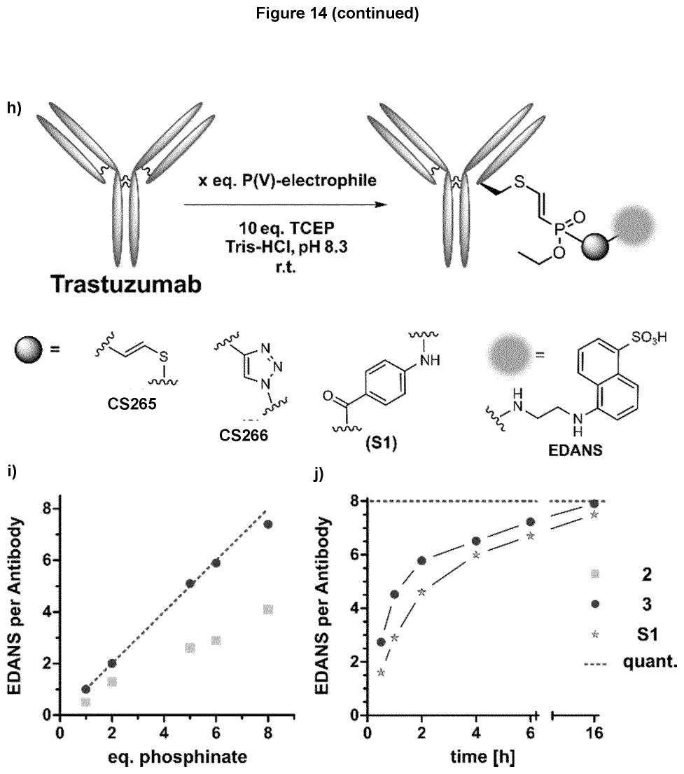

The Her2-targeting therapeutic antibody Trastuzumab was chosen to test labeling with thiovinyl- and triazole-based ethynyl phosphinates. At first, thiovinyl-phosphinate CS265 and triazolyl-phosphinate CS266 were tested. In brief, Trastuzumab (5 mg/ml in Tris-buffer pH 8.3) was reduced with 10 eq. TCEP (37° C., 30 min). Subsequently, 8 eq. of the corresponding phosphinate were added, and the reaction was allowed to proceed overnight at room temperature. Compound CS265 achieved an average labelling of 4.2 fluorophores per antibody (in another run with CS265, an average labelling of 2.9 fluorophores per antibody was determined). Compound CS266 reached almost stoichiometric labelling corresponding to a fluorophore to antibody ratio (FAR) of 7.4 (FIG. 14d). In the control experiment, where the antibody was not reduced prior to the reaction with the two phosphinates, no modification could be observed by SDS-PAGE and intact-protein MS (FIG. 14g). This already indicates good thiol selectivity. Also larger excess of reagent (50-100 eq.) did not lead to any antibody-labelling in the absence of a reducing agent.

After observing an almost stoichiometric labelling when using 8 eq. CS266, it was explored if this is a general phenomenon. Accordingly, Trastuzumab was titrated with increasing amounts of phosphinates CS265 and CS266 (FIG. 14i). Full antibody labelling with up to 5 eq. and close to stoichiometric labelling with 6 & 8 eq. was observed for phosphinate CS266. Also, compound CS265 reached approximately 50% labelling for all equivalents after the same time (FIG. 14i).

The inventors also compared the class of ethynyl-triazolyl-phosphinates with ethynyl-phosponamidates in antibody labelling. Therefore, a time-course experiment was carried out, where reduced Trastuzumab was incubated with 10 eq. of the corresponding P(V)-electrophile CS266 and S1, and the reaction was monitored over 16 h using intact-protein MS. After over-night reaction both the ethynyl phosphonamidate and the ethynyl-triazolyl-phosphinate reagent reached dose to full conversion (FAR 7.5 & 7.9, respectively) after shorter reaction times, the faster reaction-kinetics of phosphinate CS266 resulted in a higher degree of functionalisation (FIG. 14j).

Procedure for antibody labeling: The antibody Trastuzumab (5 mg/ml in the reaction buffer comprising 50 mM Tris-HCl, 150 mM NaCl and 1 mM EDTA, pH 8.3) is reacted with 8 eq. TCEP and 8 eq. of P(V) compound CS265 or CS266, which both carry a fluorophore (EDANS), for 16 h. Labeling efficiency is analyzed by SDS-PAGE and intact-protein MS. It has been shown that labeling of the antibody could be achieved by both CS265 and CS266. A fluorophore to antibody ratio (FAR) of 4.2 or 2.9 has been determined for CS265. Further, a fluorophore to antibody ratio (FAR) of 7.4 has been determined for CS266.

Reaction of non-reduced Trastuzumab with 100 eq. CS266: Trastuzumab (1 mg/ml in 50 mM Tris, 100 mM NaCl, 1 mM EDTA, pH 8.3) is reacted with 100 eq. phosphinate 3 without the addition of a reducing agent for 16 h. After removal of excess labelling reagent using 0.5 mL Zeba™ Spin Desalting Columns with 7K MWCO (Thermo Fisher Scientific, USA), the antibody was reduced with 5 mM DTT (37° C., 30 min) and analyzed via intact-protein MS. No labelling of the antibody could be detected indicating excellent cysteine selectivity of the labelling reagent (FIG. 14g).

The reaction scheme, structures of CS265 and CS266, analysis by SDS-PAGE and intact protein MS are shown in FIG. 14, which shows antibody labeling using thiovinyl- and triazole-based ethynyl-phosphinates. FIG. 14a shows the reaction scheme. Trastuzumab (5 mg/ml in reaction buffer) is reacted with 8 eq. TCEP and 8 eq. of compounds CS265 and CS266. FIG. 14b shows the structures of CS265 and CS266. FIG. 14c shows analysis of the conjugates by SDS page. FIG. 14d shows analysis of the conjugates by intact-protein MS. A fluorophore to antibody ratio (FAR) of 2.9 with CS265 and 7.4 with CS266 was determined. FIG. 14e shows exemplary deconvoluted intact-protein-MS spectra of other runs of the reaction of Trastuzumab with 1 equivalent or 8 equivalents of phosphinate CS265. FIG. 14f shows exemplary deconvoluted intact-protein-MS spectra of other runs of the reaction of Trastuzumab with 1 equivalent or 8 equivalents of phosphinate CS266. FIG. 14g shows the reaction of non-reduced Trastuzumab with 100 eq. CS266. No labelling of non-reduced Trastuzumab with CS266 could be detected using intact-protein MS. FIG. 14h shows labeling of Trastuzumab with thiovinyl-ethynyl phosphinate CS265, triazolyl-ethynyl phosphinate CS266 and ethynyl-phosphonamidate S1. FIG. 14i shows the titration of Trastuzumab with increasing amounts of phosphinates CS265 and CS266. FIG. 14j shows a time-course experiment, where reduced Trastuzumab was incubated with 10 eq. of the triazolyl-ethynyl phosphinate CS266 and ethynyl phosphonamidate S1.

Experimental procedures are given in Examples 17 and 18 below.

### Example 9: Kinetics of the Conjugate Formation Between Glutathione and EDANS-Phosphinates CS265 and CS266

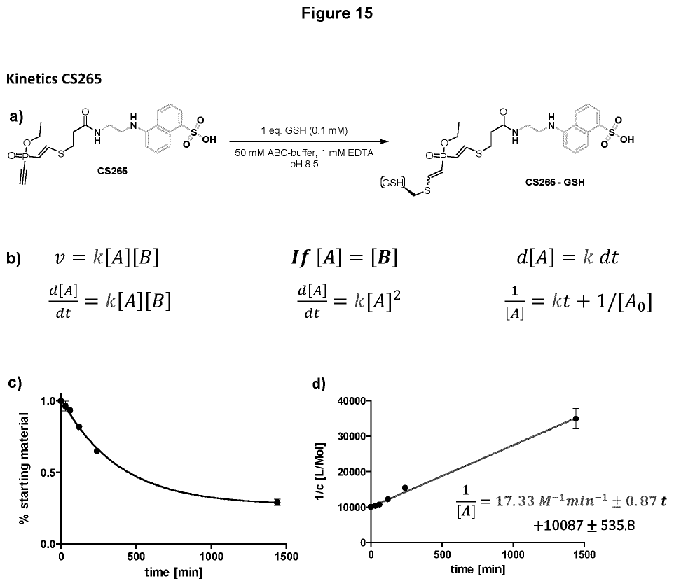

A small thiol containing fluorophore (EDANS-SH) was reacted with excess of ethyl diethynyl-phosphinate (1) and obtained the potential labelling reagent CS265 in good yield (Z-isomer, 76%) (FIG. 15). To assess the potential of this reagent for protein labelling, we determined its second order rate constant in the reaction with reduced glutathione in aqueous buffer, as described before (Kasper, M. A. et al. Vinylphosphonites for Staudinger-induced chemoselective peptide cyclization and functionalization. Chem. Sci. 10, 6322-6329 (2019); Baumann, A. L. et al. Chemically Induced Vinylphosphonothiolate Electrophiles for Thiol-Thiol Bioconjugations. J. Am. Chem. Soc. 142, 9544-9552 (2020)). Smooth conversion to a mixture of the E- and Z-isomers of the glutathione-adduct was observed.

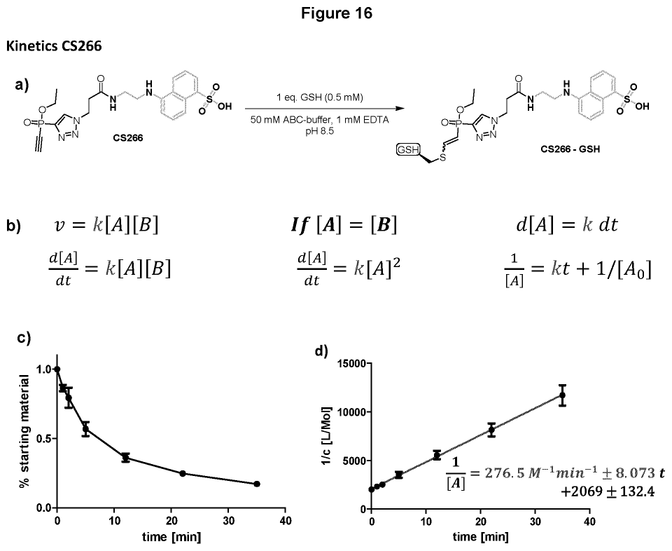

The inventors further investigated the synthesis of a triazole-based ethynyl-phosphinate starting from EDANS-N₃ and ethyl diethynyl-phosphinate (1) via Cu(I) mediated azide-alkyne cycloaddition. Using 5 eq. phosphinate allowed to obtain compound CS266 in good yield (FIG. 16). A kinetic analysis was performed using glutathione as a model thiol. Phosphinate CS266 showed accelerated reaction kinetics; the reaction with CS266 was roughly ten times faster than with CS265 (FIG. 16).

The second-order rate constant of the reaction between glutathione and EDANS-phosphinates CS265 and CS266 was determined according to the following procedure: The EDANS-phosphinate was reacted with 1 eq. of glutathione (0.5 mM) in the presence of 50 mM ABC buffer (ammonium bicarbonate buffer) and 1 mM EDTA at pH 8.5. The reactions were performed in a volume of 0.1 ml. The first sample (t=0) was drawn before the addition of glutathione. Samples were taken after 1, 2, 5, 12, 22 and 35 min (CS266) or after 30, 60, 120, 240 and 1440 min (CS265). Samples were drawn in a volume of 10 μl and immediately diluted into 200 μl of 50 mM NaOAc buffer at pH 3.5, to stop the reaction. Those samples were subjected to fluorescent HPLC analyses, injecting 50 μl each.

FIG. 15 shows the determination of the second-order rate constant of the reaction between glutathione and EDANS-phosphinate CS265. FIG. 15a shows the reaction conditions. Reactions were performed in a volume of 0.1 ml. The first sample (t=0) was drawn before the addition of glutathione. Samples were taken after 30, 60, 120, 240 and 1440 min (CS265). Samples were drawn in a volume of 10 μl and immediately diluted into 200 μl of 50 mM NaOAc buffer at pH 3.5, to stop the reaction. Those samples were subjected to fluorescent HPLC analyses, injecting 50 μl each. FIG. 15b shows the mathematic consideration for the determination of a second order rate constant with equal concentrations of the two reactants. FIG. 15c shows the concentration of starting material over time. Calculated by integration of the peaks in relation to the internal standard (EDANS). Shown are mean and error of three independent measurements.(n=3) FIG. 15d shows the graph: 1/c over time and linear plot. Slope is the second order rate constant. Shown are mean and error of three independent measurements.

FIG. 16 shows the determination of the second-order rate constant of the reaction between glutathione and EDANS-phosphinates CS266. FIG. 16a shows the reaction conditions. Reactions were performed in a volume of 0.1 ml. The first sample (t=0) was drawn before the addition of glutathione. Samples were taken after 1, 2, 5, 12, 22 and 35 min (CS266). Samples were drawn in a volume of 10 μl and immediately diluted into 200 μl of 50 mM NaOAc buffer at pH 3.5, to stop the reaction. Those samples were subjected to fluorescent HPLC analyses, injecting 50 μl each. FIG. 16b shows the mathematic consideration for the determination of a second order rate constant with equal concentrations of the two reactants. FIG. 16c shows the concentration of starting material over time. Calculated by integration of the peaks in relation to the internal standard (EDANS). Shown are mean and error of three independent measurements.(n=3) FIG. 16d shows the graph: 1/c over time and linear plot. Slope is the second order rate constant. Shown are mean and error of three independent measurements.

The results show that efficient conjugate formation between glutathione and CS265 and CS266 was achieved.

The inventors further synthesized various functionalized triazole-based ethynyl-phosphinates (Scheme 2).

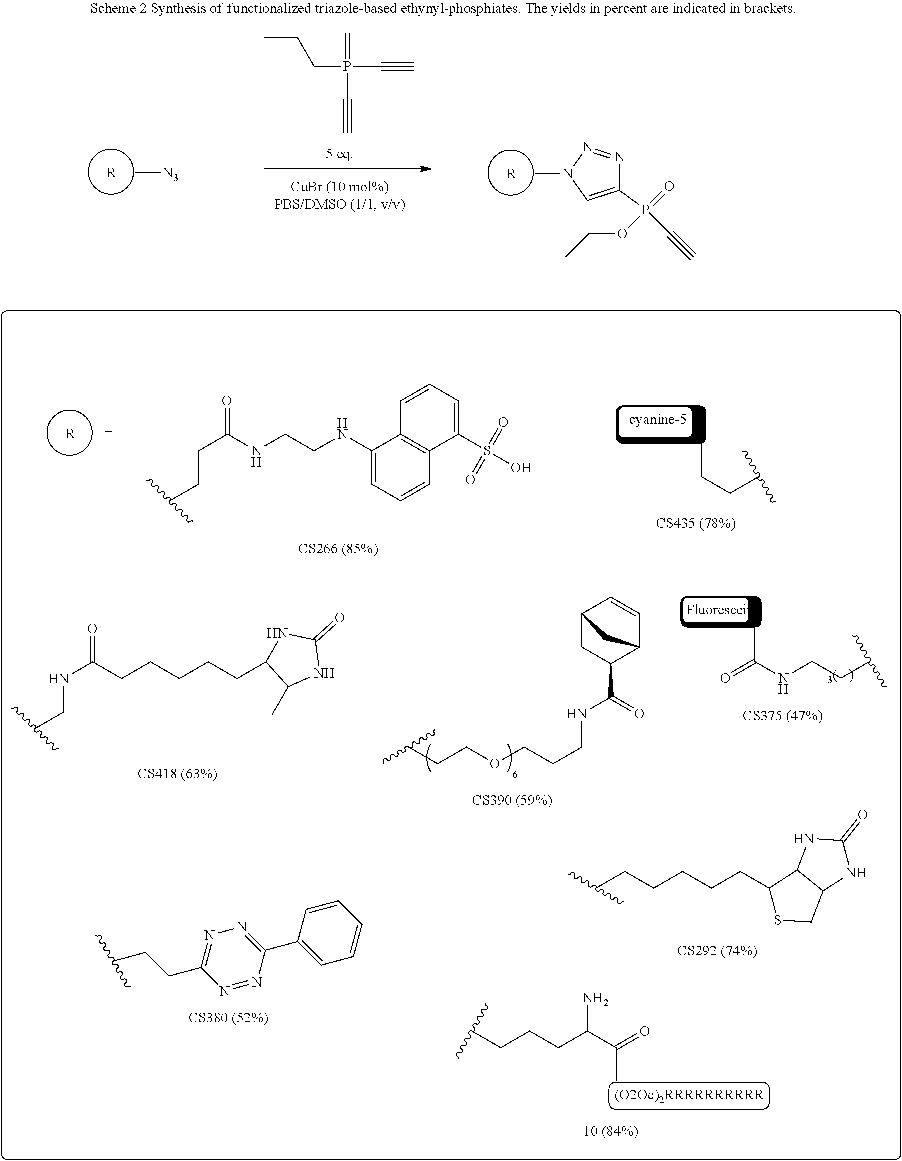

Scheme 2: Synthesis of Functionalized Triazole-Based Ethynyl-Phosphinates. The Yields in Percent are Indicated in Brackets.

Using the described procedure, triazole-based ethynyl-phosphinates reagents bearing fluorophores (CS266, CS375 & CS435), affinity-tags (CS418 & CS292) or click-handles (CS390 & CS380) were prepared in moderate to good yield. Moreover, this strategy allows for the incorporation of a triazole-based ethynyl-phosphinate into an azide-containing peptide (10).

Experimental procedures are given in Examples 17 and 18 below.

### Example 10: Stability of Triazole-Phosphinate Thiol-Adducts

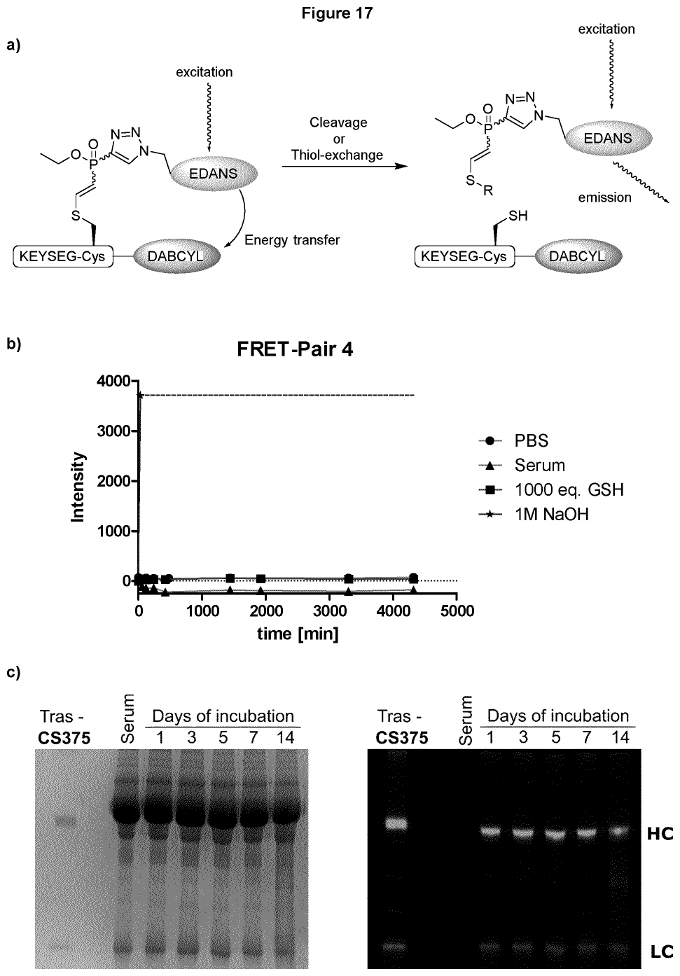

Serum-stability of the linkage is important for the successful application in antibody-drug-conjugates to prevent hazardous off-target effects. To test this, similarly to above Example 3, a fluorophore-quencher based assay was performed in order to determine the stability of a triazole-phosphinate thiol adduct (FRET-Pair 4 (F4)) comprising both DABCYL and EDANS. Stability in PBS, serum, in the presence of 1000 eq. glutathione and 1M NaOH was tested. Incubation of F4 in physiologic buffer in the presence of excess small thiols and in human serum did not show any increase in fluorescence signal, indicating excellent stability. FIGS. 17a and 17b show the structures and results. The quenched FRET-pair F4 was synthesized from phosphinate CS266 and Peptide 1 comprising DABCYL.

FIGS. 17a and 17b show a fluorophore-quencher based assay to investigate the stability of triazole-phosphinate thiol-adduct (FRET-Pair 4 (F4)). FIG. 17a shows the structure of the phosphinate linked dye-quencher conjugate and principle of the fluorescence-quencher based readout. FIG. 17b shows the results of the fluorescence measurements for conjugate F4.

Excellent stability of the triazole-phosphinate thiol-adduct in physiological buffer, human serum and in the presence of excess free thiols over time was observed. (FIG. 17b).

Further, an antibody-fluorescein conjugate using Trastuzumab and phosphinate CS375 (Trastuzumab-CS375) was synthesized, and incubated in human serum for 14 days at 37° C. No significant transfer onto other serum proteins was observed, thus confirming stability of the linkage (FIG. 17c).

FIG. 17c shows the analysis of the samples obtained after incubating the antibody fluorescein conjugate Trastuzumab-CS375 with human serum after different times of incubation.

Experimental procedures are given in Examples 17 and 18 below.

### Example 11: Rebridging of Disulfides in Antibody Using Triazolyl-Substituted Phosphinoxide

The antibody Trastuzumab was reduced using TCEP, and then rebridged using triazolyl-substituted phosphinoxide CS298 carrying a fluorescein moiety. CS298 was synthesized via Cu-click reaction of triethynyl phosphinoxide and fluorescein-azide (FAM-N₃). SDS-PAGE analysis of the rebridging reaction after 16 h showed a high degree of >85% of the re-bridged antibody.

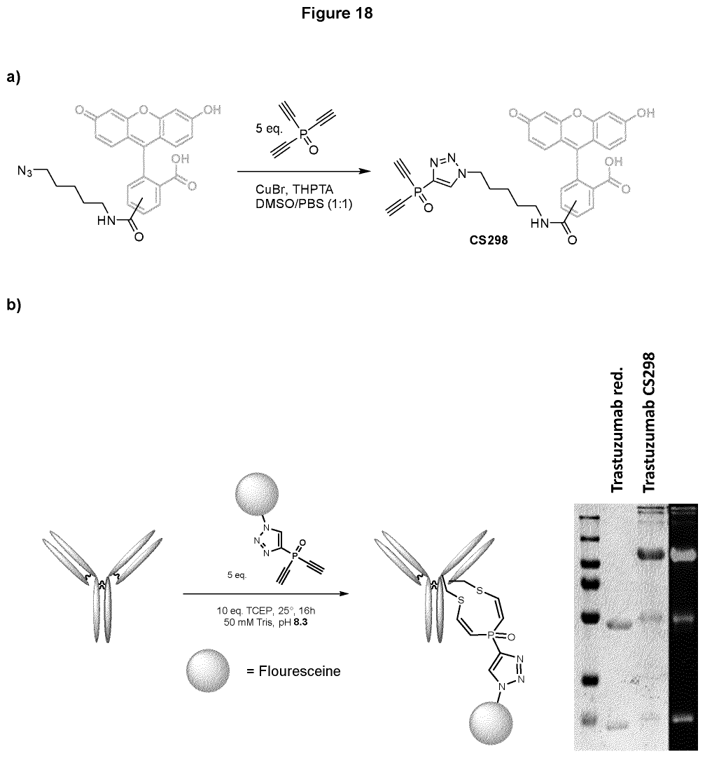

FIG. 18 shows the preparation and structure of compound CS298, and rebridging of an antibody using this compound. FIG. 18a shows the preparation of compound CS298 from fluorescein-azide (FAM-N₃) and tri-ethynyl-phosphinoxide. FIG. 18b shows rebridging of an antibody using CS298. SDS-PAGE analysis showed a high degree (>85%) of rebridged antibody.

Experimental procedures are given in Examples 17 and 18 below.

### Example 12: Protein-Labeling Using Triazole-Vinyl-Phosphinates

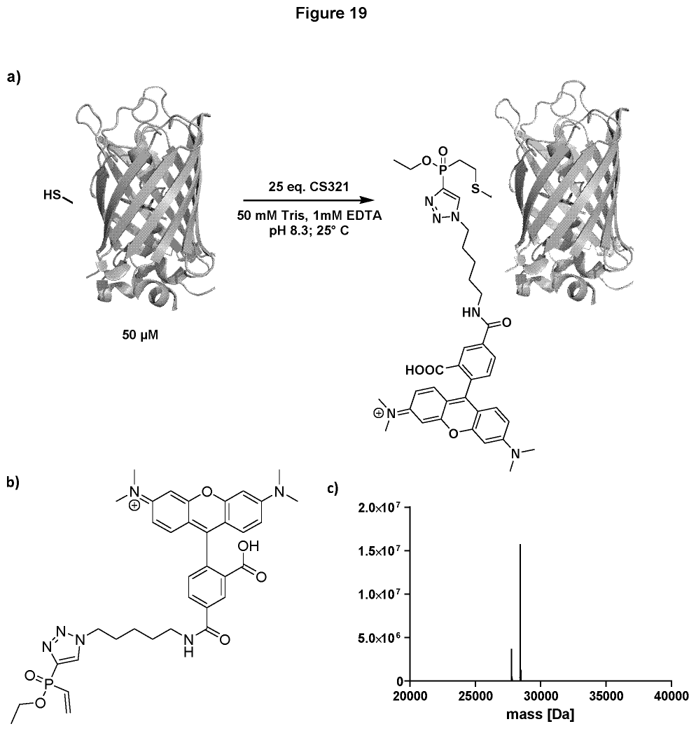

eGFP (50 μM) in a reaction buffer comprising 50 mM Tris and 1 mM EDTA is reacted with 25 eq. of triazole vinyl compound CS321 to give the corresponding labeled eGFP. The reaction scheme, the structure of CS321 and a mass spectrum are shown in FIGS. 19a, b and c. The mass spectrum was recorded after a reaction time of three days.

Experimental procedures are given in Examples 17 and 18 below.

### Example 13: Antibody Drug Conjugate

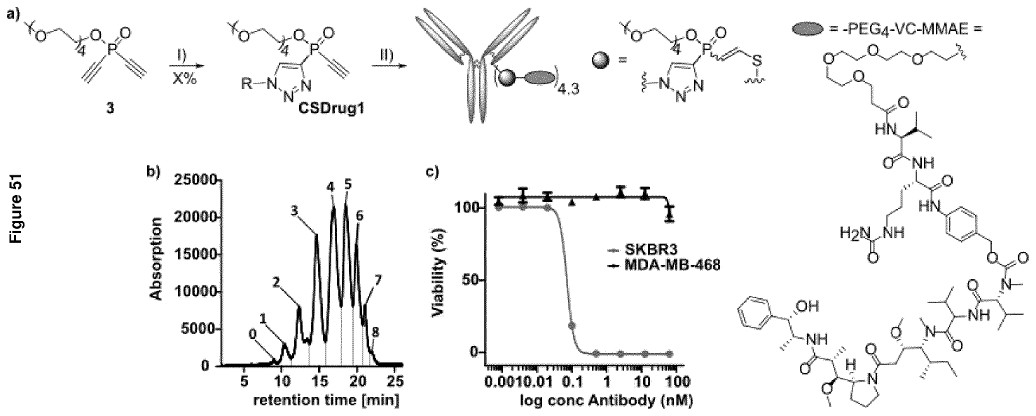

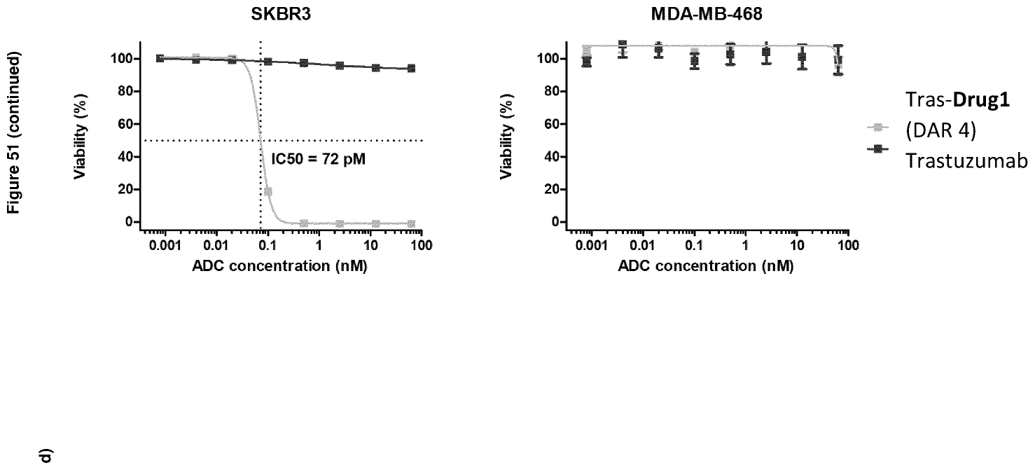

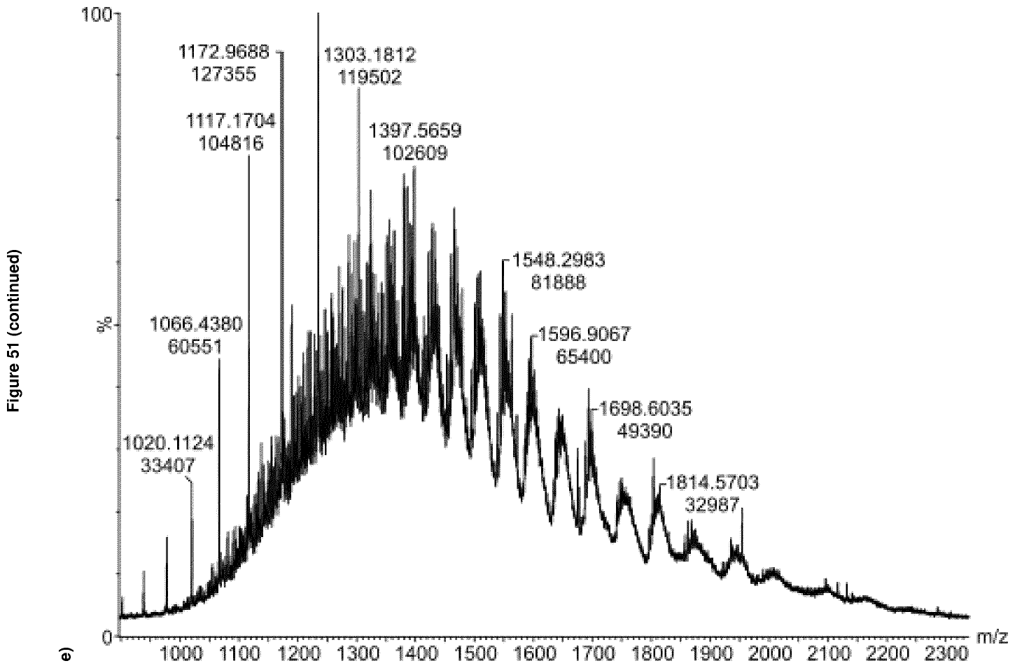

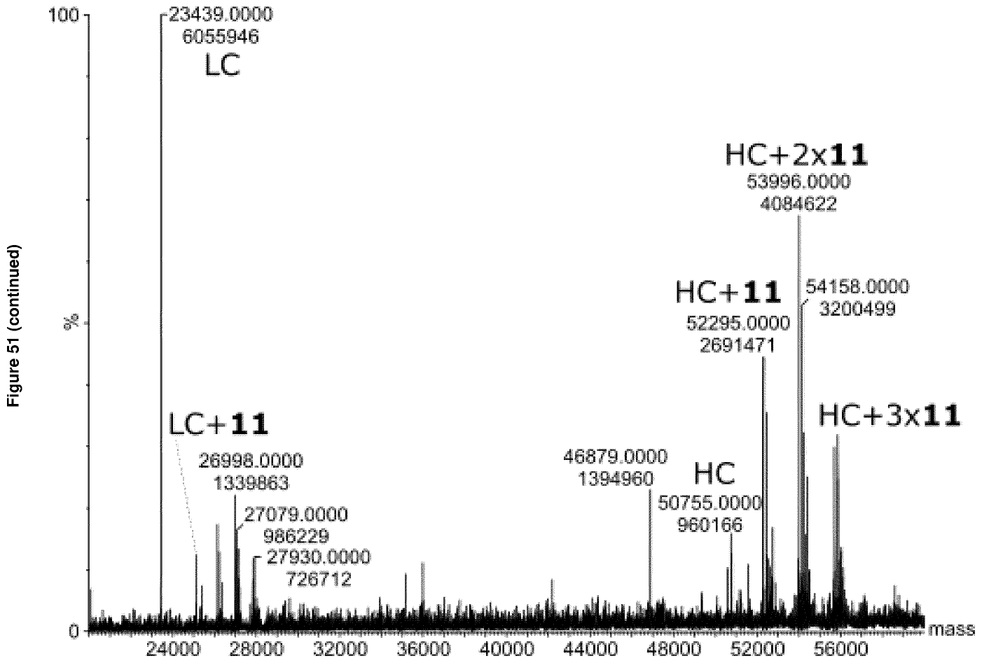

In this example, triazolyl-based ethynyl phosphinates were used for the synthesis of an antibody drug conjugate (ADC). Monomethyl-auristatin E (MMAE), a potent anti-mitotic drug that is also used in the FDA-approved ADC Vedotin®, was chosen as a payload for the ADC. Moreover, the cathepsin B cleavable Val-Cit dipeptide was included in the linker to assure free MMAE is released from the antibody after endosomal uptake. For the preparation of the ADC, mPEG4-diethynyl phosphinate 3 was used. After HPLC purification, the functionalized drug CSDrug1 was obtained in 59% yield (FIG. 51a). Initial Trastuzumab labeling experiments were performed to determine labeling efficiency with MMAE. Since the experiments using CSDrug1 showed that already after 4 hours the majority of the reagent has reacted, the reaction was terminated and checked after that time. Intact-protein mass spectrometry revealed that already 5 eq. of CSDrug1 are sufficient to reach an average drug-to-antibody-ratio (DAR) of approximately 4 after the relatively short reaction time (FIG. 51b, FIG. 51e). Hence, a scale-up of the reaction was carried out using 1 mg of Trastuzumab to allow for further biological testing. After purification via size-exclusion-chromatography (SEC), the functionalized antibody Trastuzumab-CSDrug1 was obtained in 80% yield (0.8 mg). Analysis via hydrophilic-interaction-chromatography (HIC) revealed that most of the antibody-molecules are functionalized with 3-6 drug molecules resulting in an average DAR of 4.3. (FIG. 51b) The cellular cytotoxicity of this Her-2 directed ADC was subsequently evaluated using the Her2-MDA-MB-468 and the Her2+ SKBR3 cell-lines. While proliferation of the antigen expressing cell-line could be fully inhibited by Trastuzumab-CSDrug1 from concentrations as low as 0.5 nM, antigen-negative cells were only influenced at very high concentrations of the ADC (>100 nM, FIG. 51d). These promising in-cellulo results highlight the potential of triazolyl-ethynyl-phosphinates as modular building blocks for the generation of efficacious biotherapeutics.

FIG. 51 shows the synthesis and biological evaluation of the ADC Trastuzumab-CSDrug1. FIG. 51a shows the synthetic route towards the functionalized toxic payload CSDrug1 that is used in the generation of a Trastuzumab based ADC. I) 0.2 eq. MMAE-VC-PEG₄-N₃, 10 mol % CuBr, PBS/DMSO (2:8, v/v), 4 h r.t., 59% yield. 11) 0.2 eq. Trastuzumab (5 mg/ml), 10 eq. TCEP (with respect to Trastuzumab), Tris-buffer (50 mM, pH 8.3), 1 mM EDTA, 100 mM NaCl. FIG. 51b shows the hydrophilic-interaction-chromatography of the purified ADC (Trastuzumab-CSDrug1; DAR 4.3). FIG. 51c shows the concentration dependent cellular cytotoxicity in Her2+(SKBR3, green) and Her2-(MDA-MB-468, black) cell lines. FIG. 51d shows the concentration dependent cellular cytotoxicity in Her2+(SKBR3, left) and Her2-(MDA-MB-468, right), and non-functionalized Trastuzumab as control, obtained from testing the ADC Trastuzumab-CSDrug1 in a proliferation assay. Cells were treated as described in Example 17 below with regard to the cell based anti proliferation assays with SKBR3 and MDA-MB-468. Trastuzumab-CSDrug1 shows dose-dependent toxicity against the Her2+ cell line SKBR3 with an IC50 of 72 μm. In contrast Her2-cells (MDA-MB-468) were not affected by the ADC. The non-functionalized Trastuzumab control did not show any cytotoxicity at the tested concentrations. FIG. 51e shows intact-protein MS of the crude reaction mixture of Trastuzumab-CSDrug1.

Experimental procedures are given in Examples 17 and 18 below.

### Example 14: Protein-Protein Conjugation

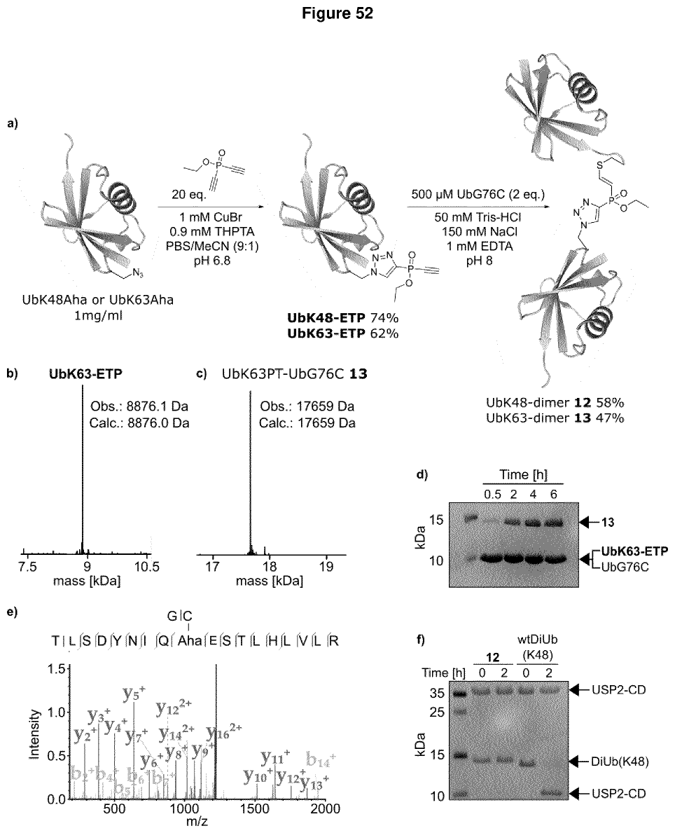

The inventors further expanded conjugation strategy using triazolyl-based ethynyl phosphinates to functionalize azide containing proteins and apply them in protein-protein conjugation. The incorporation of electrophilic vinylphosphonothiolates into ubiquitin and the subsequent application in artificial protein-ubiquitinylation has been reported previously (Baumann, A. L. et al. Chemically Induced Vinylphosphonothiolate Electrophiles for Thiol-Thiol Bioconjugations. J. Am. Chem. Soc. 142, 9544-9552 (2020)). This approach, however, comes with the drawback that anhydrous DMSO supplemented with TFA has to be used as a solvent and subsequent HPLC-purification is required. In contrast, the incorporation of a triazolyl-based ethynyl phosphinate can be achieved in physiological buffers with only a small amount of organic co-solvents. Moreover, the enhanced reactivity of the triazolyl-based ethynyl phosphinate compared to the phosphonothiolate results in superior protein-protein conjugation. To obtain an azide-containing protein, the inventors made use of an approach developed by Schneider et al. that allows for the incorporation of azidohomoalanine (Aha) into ubiquitin (Ub) via selective pressure incorporation (Schneider, T. et al. Dissecting Ubiquitin Signaling with Linkage-Defined and Protease Resistant Ubiquitin Chains. Angew. Chemie Int. Ed. 53, 12925-12929 (2014)). Following this procedure, the two Ub-mutants UbK48Aha and UbK63Aha were expressed for subsequent modification with ethyl diethynyl-phosphinate (1) (Example 17 under Expression of UbK48Aha and UbK63Aha). Compared to the small-molecule experiments, the synthetic procedure was slightly adapted (Example 17 under EETP-functionalization of UbK48Aha and UbK63Aha). Ub-mutants were re-buffered to PBS (pH 6.9) at a concentration of 1 mg/ml (115 μM) followed by the addition of ethyl diethynyl-phosphinate (1) (20 eq., 2.3 mM final concentration). CuBr and THPTA were dissolved in acetonitrile at a concentration of 50 mM and the pre-formed Cu(I)-complex was subsequently added to the Ub-phosphinate mixture to a final acetonitrile content of 5%. After 5 h reaction time full conversion of the Aha-mutants was observed via intact-protein MS. Excess of copper, ligand and phosphinate were removed via dialysis against PBS supplemented with 5 mM EDTA for 16 h, followed by size exclusion chromatography (SEC) to obtain triazolyl-phosphinate-modified UbK48ETP and UbK63ETP in 74% and 62% yield, respectively (FIG. 52a).

Next, the inventors used these protein-electrophiles in protein-protein conjugation. Therefore, both UbK48ETP and UbK63ETP were subjected to the bioconjugation with an UbG76C mutant. The reactions were carried out in Tris-buffer at pH 8 and monitored via intact-protein-MS and SDS-PAGE (FIG. 52d & 53). The starting material was fully consumed after 6 h reaction time and the corresponding Ub-dimers (12 & 113) were purified via SEC.

To verify the linkage and cysteine-selectivity, the purified dimers were digested using trypsin and analyzed via bottom-up proteomics. Using both conventional and dedicated proteomics software allowed to verify the correct linkage sites, with no detectable side-reactivity (FIG. 52e & 54) (Dorfer, V. et al. M S Amanda, a universal identification algorithm optimized for high accuracy tandem mass spectra. J. Proteome Res. 13, 3679-3684 (2014); G6tze, M. et al. StavroX-A software for analyzing crosslinked products in protein interaction studies. J. Am. Soc. Mass Spectrom. 23, 76-87 (2012)). Furthermore, the protease-resistance of the linkage-specifically synthesized ubiquitin dimers against deubiquitinating enzymes (DUBs) was investigated. While the DUB USP2 was able to completely cleave K48-linked wild-type diubiquitin no degradation of our synthetic analogue was observed. (FIG. 52f and 61).

FIG. 52 shows the protein-protein conjugation according to Example 14. FIG. 52a shows the synthetic strategy to obtain electrophilic ubiquitin from site-selectively installed K→Aha mutants. FIG. 52b shows an intact protein-MS of the UbK63ETP. FIG. 52c shows an intact-protein MS of the artificial Ub-dimer 13. FIG. 52d shows the time course of the conjugation of UbK63ETP to UbG76C monitored by SDS-Page. FIG. 52e shows the MS/MS-spectrum identifying the linkage-site of 13. FIG. 52f shows the SDS-PAGE analysis of 12 and wtUbiquitin incubated with USP2.

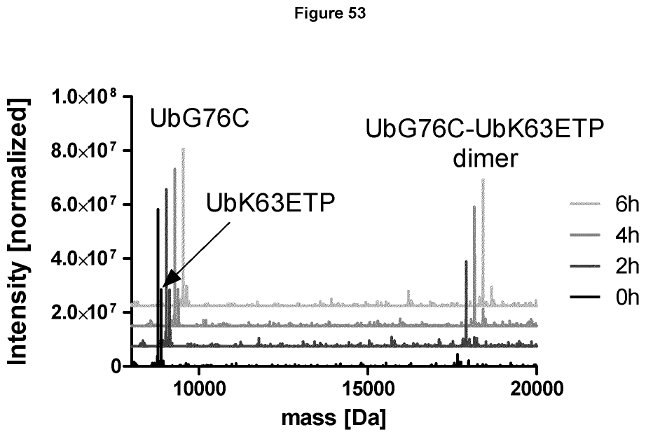

FIG. 53 shows the time course of the UbG76C-UbK63ETP dimer formation. UbK63ETP (200 μM) was reacted with 2.5 eq. freshly reduced UbG76C as described in Example 14 under ETP-functionalization of UbK48Aha and UbK63Aha. After 0, 2, 4 and 6 hours a sample was drawn and analyzed via intact-protein MS. Deconvoluted mass spectra were normalized to the UbG76C peak, plotted and stacked using Graphpad Prism 5 software.

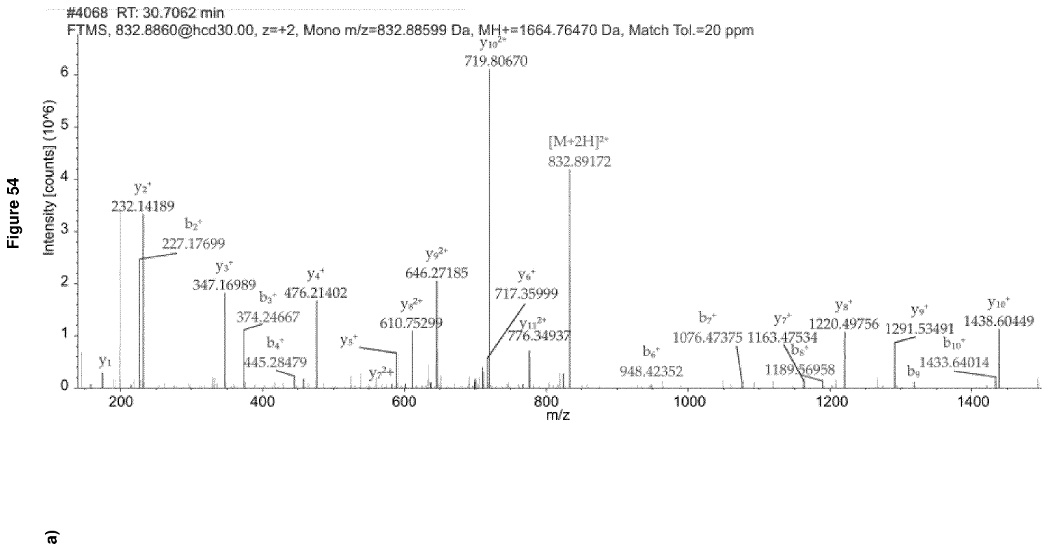

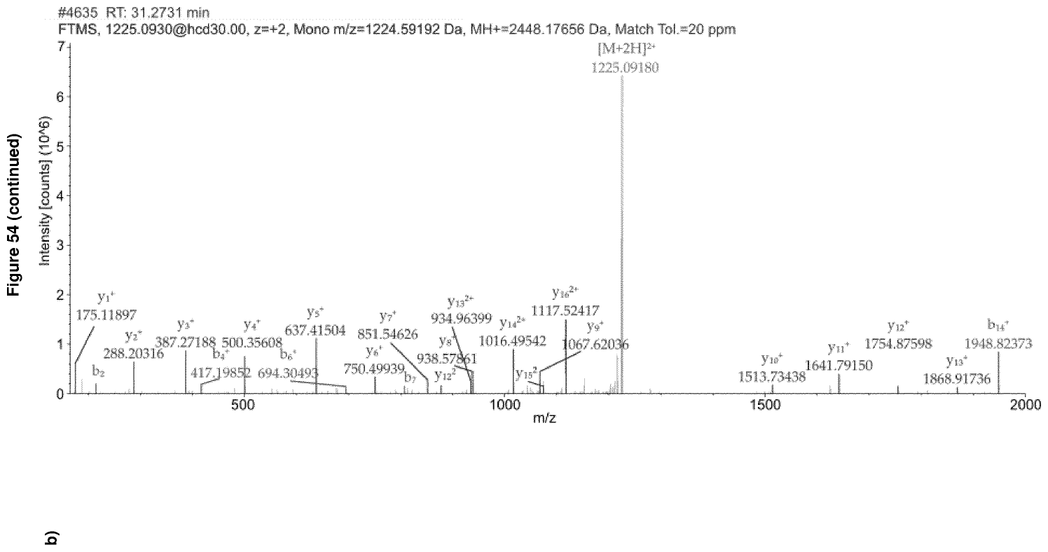

FIG. 54 shows the MS/MS-analysis of artificial ubiquitin dimers. FIG. 54a shows the MS/MS-analysis of UbK48-ETP-UbG76C. FIG. 54b shows the MS/MS-analysis of UbK63-ETP-UbG76C. For the MS/MS analysis, UbK48-ETP-UbG76C (a) and UbK63-ETP-UbG76C (b) dimers were prepared as described in Example 17 under ETP-functionalization of UbK48Aha and UbK63Aha, and analyzed as described in Example 16 under Proteomics Data Analysis—Ubiquitin dimers using Proteome Discoverer (v. 2.5.0.400) and MS Amanda 2.0. Exemplary spectra show the best scoring peptide-spectrum-match (PSM) identifying the correct linkage site.

FIG. 61 shows the SDS-PAGE analysis of dimer 12 incubated with USP2-CD.

Experimental procedures are given in Examples 17 and 18 below.

### Example 15: Proteomwide Cysteine Labelling

Having demonstrated the utility of ETP-reagents for cysteine selective protein modification, the inventors tested if this compound-class can be applied to proteome-wide cysteine labeling and profiling in whole-cell lysates. To test this, HEK-293 cell-lysate (1 mg/ml in PBS pH 7.4) was treated with increasing amounts of phosphinate CS375 for 45 minutes at room-temperature before analysis via SDS-PAGE and in-gel fluorescence (FIG. 55a). Coomassie staining was used to control for equal loading. We observed concentration dependent labeling of the lysate, where already at 30 μM 5 a decent fluorescent signal was observed.

To identify the sites of labeling, the inventors made use of desthiobiotin-PT CS418 and adapted a workflow that allows for the unbiased analysis of electrophile selectivity on proteome scale, recently developed by Zanon et al (Zanon, P. R. A. et al. Profiling the proteome-wide selectivity of diverse electrophiles. https://fragpipe.nesvilab.org (2021) doi:10.26434/CHEMRXIV.14186561.V1). In brief, HEK293-cell lysate (1 mg/ml, PBS pH 7.4) was treated with 200 μM of phosphinate CS418 for 45 minutes at room temperature. Afterwards the proteins were precipitated using ice-cold acetone and enriched using streptavidin-beads. After tryptic digestion and elution of the labeled peptides, the samples were analyzed by high-resolution liquid-chromatography couple tandem mass spectrometry (LC-MS/MS). To identify the mass of modification, the inventors performed an open search in MS-Fragger using the previously optimized settings (Zanon, P. R. A. et al. Profiling the proteome-wide selectvity of diverse electrophiles. https://fragpipe.nesvilab.org (2021) doi:10.26434/CHEMRXIV.14186561.V1). Pleasingly, the most prominent modification in all three replicates corresponds to the expected mass of an alkylation with phosphinate 6 (Δmₑₓₚ, 27137 PSMs). Moreover, the modification-mass plus oxidation (Δm_{Ox}, 1747 PSMs), formylation (Δm_{f}, 2005 PSMs) and carbamidomethylation (Δm_{CAM}, 4546 PSMs) were detected as well (FIG. 55d).

Having identified the modification mass, the next step is to analyse the amino acid selectivity. Therefore, the inventors performed an offset-mass search using Δmₑₓₚ as the modification mass. In this search every amino acid is considered as a potential site of modification, allowing for an unbiased investigation.

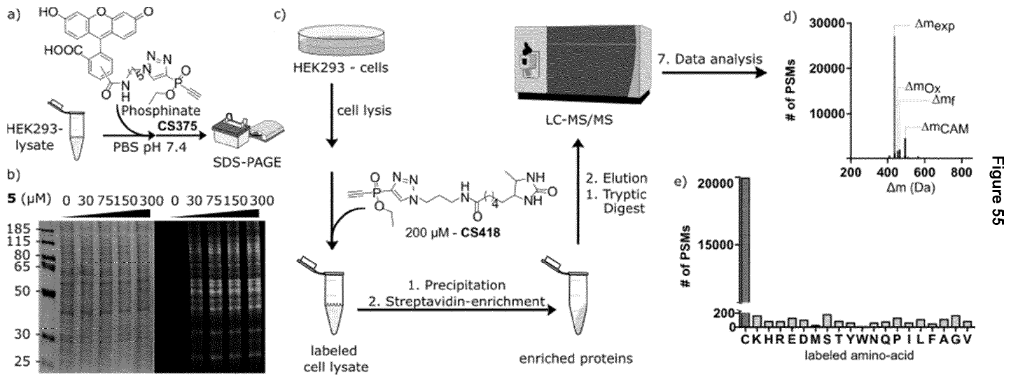

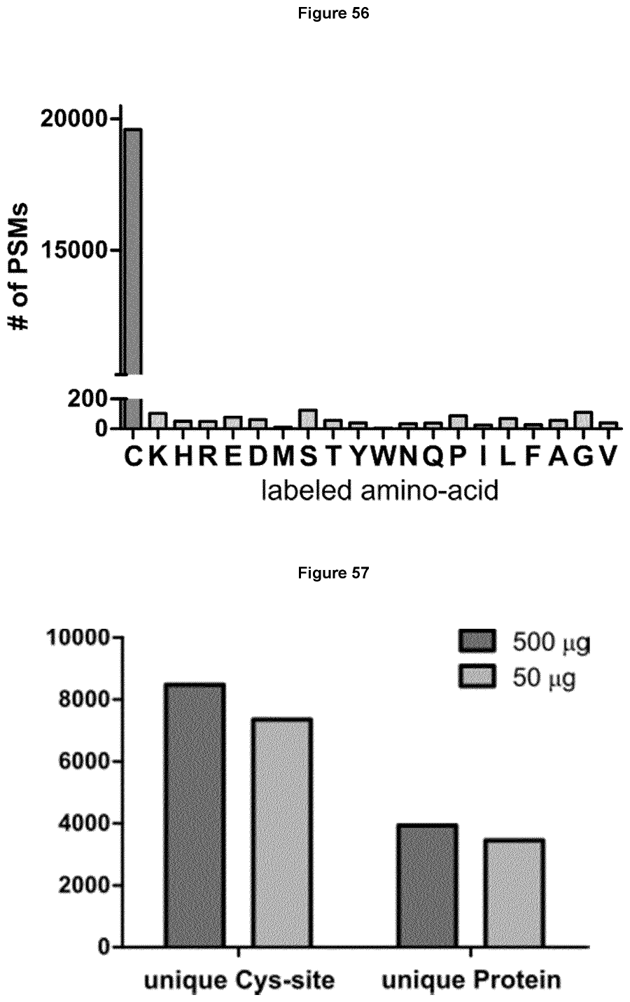

The obtained results were filtered to contain only PSMs that are contained within 1% peptide level false discovery rate (FDR), to assure for good data quality. Moreover, only spectra, where MS-Fragger was able to assign a single modification site were considered in the analysis. Out of 22102 spectra that matched the aforementioned criteria, 20429 (92%) were located at a cysteine residue (FIG. 55e). The remaining spectra were distributed among all other 19 proteinogenic amino acids, with none of them reaching more than 150 spectral matches (<0.7%). Taking a closer look at the results, it was found that out of the 1673 spectra that were not localized to a cysteine, 817 are assigned to an amino acid residue adjacent to a cysteine. Unambiguous site-localization is often difficult when MS/MS spectra do not contain enough fragment peaks. To account for this uncertainty, a delta-score >1 was applied (the score-difference between best and second-best modification site). From the remaining spectra (19595 PSMs) 95% were found to be modified on a cysteine (FIG. 56). These findings further underline the excellent cysteine selectivity of ETP-electrophiles.

Finally, the inventors performed a conventional search using MS Amanda (Dorfer, V. et al. M S Amanda, a universal identification algorithm optimized for high accuracy tandem mass spectra. J. Proteome Res. 13, 3679-3684 (2014)) applying the obtained parameters for desthiobiotin-PT CS418 (modification-mass: 438.2136; modification-site: Cys) as a variable modification (see Example 16 under Proteomics Data Analysis for details). In total, 8661 unique labeled cysteine sites derived from 3978 proteins could be identified from three independent replicates. Interestingly we found, that even at a lower cell lysate concentration along with a lower total proteomic input (0.1 mg/ml, 50 μg total) still 7023 unique cysteine sites could be detected (FIG. 57).

FIG. 55 shows the Investigation of proteome-wide cysteine reactivity of ETP-electrophiles. FIG. 55a shows the workflow for the labelling of whole-cell lysate using the fluorescent phosphinate CS375 and subsequent analysis. FIG. 55b shows the SDS-PAGE analysis of cell lysate treated with increasing electrophile concentration. FIG. 55c shows the illustration of the workflow used for the unbiased analysis of electrophile selectivity via MS-based proteomics. FIG. 55d shows the histogram of the modifications detected in MS-Fragger open search across three replicates. Δm of phosphinate CS418 is highlighted in green. ox=oxidation (+15.99 Da), f=formylation (+27.99 Da), CAM=carbamidomethylation (+57.02 Da). FIG. 55e shows the abundance of identified modification sites when using Δmₑₓₚ as offset-mass. #of PSMs represents the sum of three replicates.

FIG. 56 shows the proteome wide amino acid selectivity applying a ΔScore >1. The proteome-wide amino acid selectivity was determined as described in Example 16 under Proteomics Data Analysis and Example 17 under Sample preparation for proteome-wide cysteine profiling with an additional ΔScore filter of >1.

FIG. 57 shows the proteome-wide cysteine-profiling. Comparison of the protein input for cysteine proteomics using phosphinate CS418. Data was analyzed using Proteome Discoverer (v. 2.5.0.400) and MS Amanda 2.0. Values shown are the sum of three replicates.

Experimental procedures are given in Examples 17 and 18 below.

### Example 16: General Information

### Chemicals and Solvents

Chemicals and solvents were purchased from Merck (Merck group, Germany), TCI (Tokyo chemical industry CO., LTD., Japan) and Acros Organics (Thermo Fisher scientific, USA) and used without further purification. Dry solvents were purchased from Acros Organics (Thermo Fisher scientific, USA). Aminoacids and resins for SPPS were purchased from Novabiochem (Merck, USA) or Iris Biotech GmbH (Germany).

### Flash- and Thin Layer Chromatography

Flash column chromatography was performed, using NORMASIL 609 silica gel 40-63 μm (VWR international, USA). Glass TLC plates, silica gel 60 W coated with fluorescent indicator F254s were purchased from Merck (Merck Group, Germany). Spots were visualized by fluorescence depletion with a 254 nm lamp or manganese staining (10 g K₂CO₃, 1.5 g KMnO₄, 0.1 g NaOH in 200 ml H₂O), followed by heating.

### Semi-vreDarative HPLC

Semi-preparative HPLC was performed on was performed on a Shimadzu prominence HPLC system (Shimadzu Corp., Japan) with a CBM20A communication bus module, a FRC-10A fraction collector, 2 pumps LC-20AP, and a SPD-20A UVNIS detector, using a VP250/21 Macherey-Nagel Nucleodur Cie HTec Spum column (Macherey-Nagel GmbH & Co. Kg, Germany). The following gradient was used: (A=H₂O+0.1% TFA, B=MeCN++0.1% TFA), flow rate 10 ml/min, 5% B 0-5 min, 5-99% B 5-65 min, 99% B 65-75 min.

### NMR

NMR spectra were recorded with a Bruker Ultrashield 300 MHz spectrometer and a Bruker Avance III 600 MHz spectrometer (Bruker Corp., USA) at ambient temperature. Chemical shifts δ are reported in ppm relative to residual solvent peak (CDCl₃: 7.26 [ppm]; DMSO-d₆: 2.50 [ppm]; acetone-d₆: 2.05 [ppm]; CD3CN 1.94 [ppm]; 4.79 D20 [ppm] for ¹H-spectra and CDCl₃: 77.16 [ppm]; DMSO-d₆: 39.52 [ppm]; acetone-d₆: 29.84 [ppm]; CD₃CN 1.32 [ppm]; for ¹³C-spectra. Coupling constants J are stated in Hz. Signal multiplicities are abbreviated as follows: s: singlet; d: doublet; t triplet; q: quartet; m: multiplet.

### UPLC-UV/MS

UPLC-UV/MS traces were recorded on a Waters H-class instrument equipped with a quaternary solvent manager, a Waters autosampler, a Waters TUV detector and a Waters Acquity QDa detectorwith an Acquity UPLC BEH C18 1.7 μm, 2.1×50 mm RP column with a flow rate of 0.6 mL/min (Waters Corp., USA). The following gradient was used: A: 0.1% TFA in H2O; B: 0.1% TFA in MeCN. 5% B 0-0.5 min, 5-95% B 0.5-3 min, 95% B 3-3.9 min, 5% B 3.9-5 min. Also, the following gradients were used: Gradient A: 0.1% TFA in H₂O; B: 0.1% TFA in MeCN. 5% B 0-1.5 min, 5-95% B 1.5-13 min, 95% B 13-13.9 min, 5% B 13.9-15 min. Gradient B: 0.1% TFA in H₂O; B: 0.1% TFA in MeCN. 5% B 0-0.5 min, 5-95% B 0.5-3 min, 95% B 3-3.9 min, 5% B 3.9-5 min.

### SPPS

SPPS was carried out manually or on a Tribute-UV peptide synthesizer (Protein technologies, USA) via standard Fmoc-based protocols.

### HR-MS

High resolution ESI-MS spectra were recorded on a Waters H-class instrument equipped with a quaternary solvent manager, a Waters sample manager-FTN, a Waters PDA detector and a Waters column manager with an Acquity UPLC protein BEH C18 column (1.7 μm, 2.1 mm×50 mm). Samples were eluted with a flow rate of 0.3 mL/min. The following gradient was used: A: 0.01% FA in H2O; B: 0.01% FA in MeCN. 5% B: 0-1 min; 5 to 95% B: 1-7 min; 95% B: 7 to 8.5 min. Mass analysis was conducted with a Waters XEVO G2-XS QTof analyzer.

### Intact Protein MS

Intact proteins were analyzed using a Waters H-class instrument equipped with a quaternary solvent manager, a Waters sample manager-FTN, a Waters PDA detector and a Waters column manager with an Acquity UPLC protein BEH C4 column (300 Å, 1.7 μm, 2.1 mm×50 mm). Proteins were eluted with a flow rate of 0.3 mL/min. The following gradient was used: A: 0.01% FA in H2O; B: 0.01% FA in MeCN. 5-95% B 0-6 min. Mass analysis was conducted with a Waters XEVO G2-XS QTof analyzer. Raw data was analyzed with MaxEnt 1.

### Protein Concentration Determination

Protein concentrations were determined by absorption spectroscopy measurements at 280 nm using the extinction coefficient and molecular weight of the protein on a NanoDrop ND-1000. In addition or as alternative concentrations were determined by BCA assay (Thermo Fisher Scientific, USA) according to the manufacturers protocol.

### Protein Purification

Protein purification was accomplished either with an ÄKTA FPLC or with a BioRad NGC system as stated below.

### CD-Spectroscopy

CD-spectroscopy was measured on a Jasco J-720 spectropolarimeter at 25° C. and parameters set to: measured wavelength range 190-260 nm; data pitch of 0.1 nm; continuous scanning mode; 100 nm/min scanning speed; 1 sec. response; 1.0 nm band width; 0.1 cm cell length; 10 accumulations

### Protein MS/MS

Peptide mixtures after tryptic digest were analyzed by a reversed-phase capillary liquid chromatography system (Dionex Ultimate 3000 NCS-3500RS Nano, Thermo Scientific) connected to an Orbitrap Fusion mass spectrometer (Thermo Fisher Scientific, Germany). For sample loading a PepMap C-18 trap-column (Thermo Fischer Scientific) of 0.075 mm ID×50 mm length, 3 μm particle size, 100 Å pore size was used. The loading mobile phase A contained 1% acetonitrile and 0.1% TFA acid in water, and mobile phase B 0.1% TFA acid in acetonitrile.LC separation was performed with a 200 cm ρPAC™ column (Pharma-Fluidics, Ghent, Belgium) at an eluent flow rate of 750 nL/min using a gradient of 4-50% B in 59 min. The separation mobile phase A contained 0.1% formic acid in water, and mobile phase B 0.1% formic acid in acetonitrile. FT survey scans were acquired in a range of 350 to 1500 m/z with a resolution of 120000 (FWHM) and an AGC target value of 4e5. Precursor ions with charge states 2-5 were isolated with a mass selecting quadrupole (isolation window 1.2 m/z) with 10 sec dynamic exclusion. Precursor ions were fragmented using stepped higher-energy collisional dissociation (HCD). Stepped HCD MS/MS spectra were acquired with 27-30-33% normalized collision energy (NCE). The maximum injection time was set to 54 ms to collect 2e4 precursor ions. Fragment ion spectra were acquired in the Orbitrap with a resolution of 15000 (FWHM).

### Analytical Hydrophilic Interaction Chromatography (HIC)

Analytical HIC of the ADCs was conducted on a Shimadzu LC20AT with a DAD detector, using a TSKgel Butyl-NPR; 4.6 mm ID×10 cm, 2.5 micrometer (TOSOH) with a flow rate of 0.5 mL/min. Separation of different ADC/mAb populations have been achieved during a 30 minute gradient Solvent A: 25 mM Na—P-Puffer pH 7=1,5M (NH₄)₂SO₄; Solvent B: 25 mM Na—P-Puffer pH 7 +20% isopropylalcohol. (0-20 min 0-100% B, 20-25 min 100% B, 25-30 min 100-0% B). 10 μg ADC where loaded onto the column for HIC analysis. UV chromatograms were recorded at 220 and 280 nm. Quantification of different species was achieved after integration of the peak area at 220 nm

### LC-MS/MS

LC-MS analysis was performed using an UltiMate 3000 RSLC nano LC system coupled on-line to an Orbitrap Fusion mass spectrometer (Thermo Fisher Scientific). For sample loading a PepMap C-18 trap-column (Thermo Fischer Scientific) of 0.075 mm ID×50 mm length, 3 μm particle size and 100 Å pore size was used. The loading mobile phase A contained 1% acetonitrile and 0.05% TFA acid in water, and mobile phase B 0.05% TFA acid in acetonitrile. Reversed-phase separation was performed using a 50 cm analytical column (in-house packed with Poroshell 120 EC-C18, 2.7 μm, Agilent Technologies) with mobile phase A contained 0.1% formic acid in water, and mobile phase B 0.1% formic acid in acetonitrile using a 45 minutes gradient (4% B 0-5 minutes; 5-40% B in 5-30 minutes; 40% B 30-35 minutes; 40-50% B 35-38 minutes; 50% B 38-40.5 minutes; 50-80% B 40.5-41 minutes; 80% B 41-44 minutes; 80-4% B 44-45 minutes) or a 93 minutes gradient (4-5% B 0-8 minutes; 5-25% B in 8-74 minutes; 25-28% B 74-80 minutes; 28-31% B 80-86 minutes; 31-36% B 86-92 minutes; 36-40% B 92-95 minutes; 40-50% B 95-96 minutes; 50-80% B 96-101 minutes; 80% B 101-104 minutes; 80-4% B 104-104.1 minutes).

Data was acquired using survey scans in a range of 375 to 1500 m/z with a resolution of 120000 and an AGC target value of 4e5. Precursor ions with charge states 2-5 were isolated with a mass selecting quadrupole (isolation window 1.6 m/z) with 10 sec dynamic exclusion (40 seconds for 93 minutes gradient). Precursor ions were fragmented using higher-energy collisional dissociation (HCD) applying a normalized collision energy (NCE) of 30. The maximum injection time was set to 54 ms to collect 2e6 precursor ions. Fragment ion spectra were acquired in the Orbitrap with a resolution of 30000 (FWHM).

### Proteomics Data Analysis

Ubiquitin dimers: The FASTA-file was modified to contain a wtUb and a UbK63A or UbK48A mutant. Analysis was done in Proteome Discoverer (v. 2.5.0.400) using MS Amanda 2.0 (Dorfer, V. et al. M S Amanda, a universal identification algorithm optimized for high accuracy tandem mass spectra. J. Proteome Res. 13, 3679-3684 (2014)) as a search engine using the following settings: MS1 accuracy: 6 ppm; MS2 accuracy: 20 ppm; used enzyme: trypsin; max. missed cleavages: 3; max. dynamic modifications: 4; peptide mass: 350 5000 Da; dynamic modifications: ETP+Ub-Cterm (A, +375.077 Da).

Proteome-wide reactivity of Desthiobiotin-ETP CS418: To determine the modification mass and amino acid selectivity, data was analyzed as described by Zanon et al. Zanon, P. R. A. et al. Profiling the proteome-wide selectivity of diverse electrophiles. https://fragpipe.nesvilab.org (2021) doi:10.26434/CHEMRXIV.14186561.V1 The closed search was performed in Proteome Discoverer (v. 2.5.0.400) using MS Amanda 2.0 (Dorfer, V. et al. M S Amanda, a universal identification algorithm optimized for high accuracy tandem mass spectra. J. Proteome Res. 13, 3679-3684 (2014)) as a search engine using the following settings: MS1 accuracy: 8 ppm; MS2 accuracy: 20 ppm; used enzyme: trypsin; max. missed cleavages: 3; max. dynamic modifications: 4; peptide mass: 350-5000 Da; dynamic modifications: carbamidomethylation (cysteine, +57.021 Da), oxidation (methionine, +15.995 Da), formylation (N-term, +27.995 Da), DTB-ETP (cysteine, +438.214 Da). For FDR calculation Percolator was used with a target FDR of 1%.

### Example 17: Experimental Procedures

Stability Studies of the Dabcyl-EDANS Adducts (in Accordance with Examples 3 and 10)

Stability studies were conducted in 96-well plate (Corning 3615, black with clear, flat bottom) at least in triplicates. 5 μl of a 200 μM Stock solution of the Dabcyl-EDANS conjugates and 95 μl of the respective test solutions were added to each well. Human serum was purchased from Sigma Aldrich. Glutathione was dissolved at a concentration of 10 mM in PBS and the pH was adjusted to 7.4. 0.1 mM NaOH studies were conducted at 200 μM, neutralized to pH 7 and diluted to 10 μM before fluorescence measurements. Fluorescence was measured on a Tecan Safire plate reader. Excitation: 360 nm, emission: 508 nm, bandwidth: 5 nm at 20° C.

eGFP C70M S147C production (in accordance with Example 4)

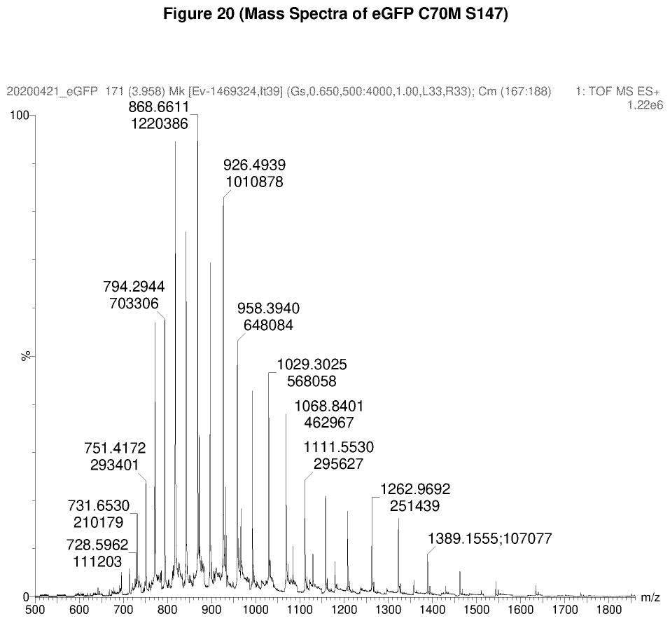

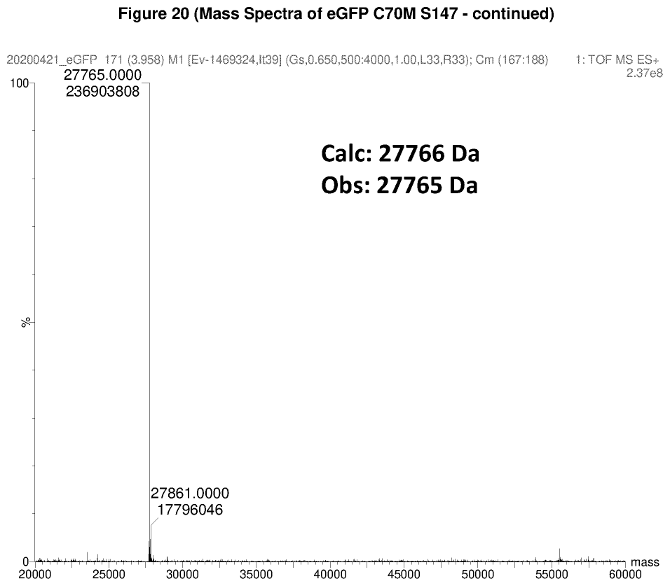

The eGFP mutant C70MS147C was expressed and purified as described in the literature (Kasper, M.; Glanz, M.; Stengl, A.; Penkert, M.; Klenk, S.; Sauer, T.; Schumacher, D.; Helma, J.; Krause, E.; Cardoso, M. C.; Leonhardt, H.; Hackenberger, C. P. R. Cysteine-Selective Phosphonamidate Electrophiles for Modular Protein Bioconjugations. Angew. Chemie Int. Ed. 2019, 58 (34), 11625-11630. https://doi.org/10.1002/anie.201814715). Aliquots were shock-frozen and stored at −80° C. until further use. Mass spectra of eGFP C70M S147C are shown in FIG. 20.

Protein sequence: His-tag highlighted in italics; protease cleavage site highlighted with subscripts; eGFP typed in normal letters; C70M and S147C highlighted in bold and subscript:

MGSSHHHHHHSSGLVPRGSHMGSIQMVSKGEELFTGVVPILVELDGDVN
GHKFSVSGEGEGDATYGKLTLKFICTTGKLPVPWPTLVTTLTYGVQMFS
RYPDHMKQHDFFKSAMPEGYVQERTIFFKDDGNYKTRAEVKFEGDTLVN
RIELKGIDFKEDGNILGHKLEYNYNCHNVYIMADKQKNGIKVNFKIRHN
IEDGSVQLADHYQQNTPIGDGPVLLPDNHYLSTQSALSKDPNEKRDHMV
LLEFVTAAGITLGMDELYK

Protein-Modification with Diethynyl-Phosphinates (in Accordance with Example 4)

The protein of interest dissolved in PBS (pH 7.4) was incubated with 10 eq. phosphinate for 10 to 60 minutes at room temperature. Excess reagent was removed using 0.5 mL Zeba™ Spin Desalting Columns with 7K MWCO (Thermo Fisher Scientific, USA). Complete labeling was verified by intact protein MS and the modified protein was stored at −20° C. until further usage.

eGFP C70M S124C-1 (in accordance with Example 4)

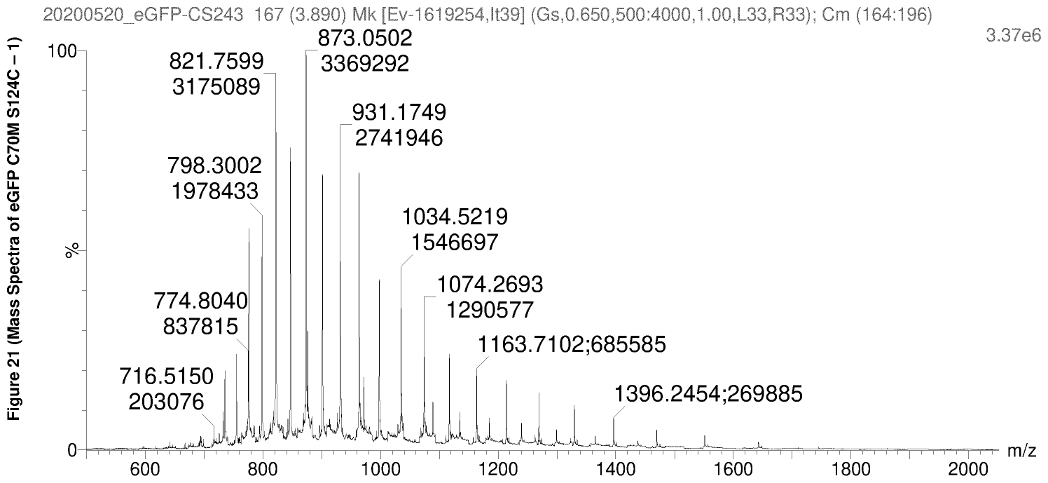

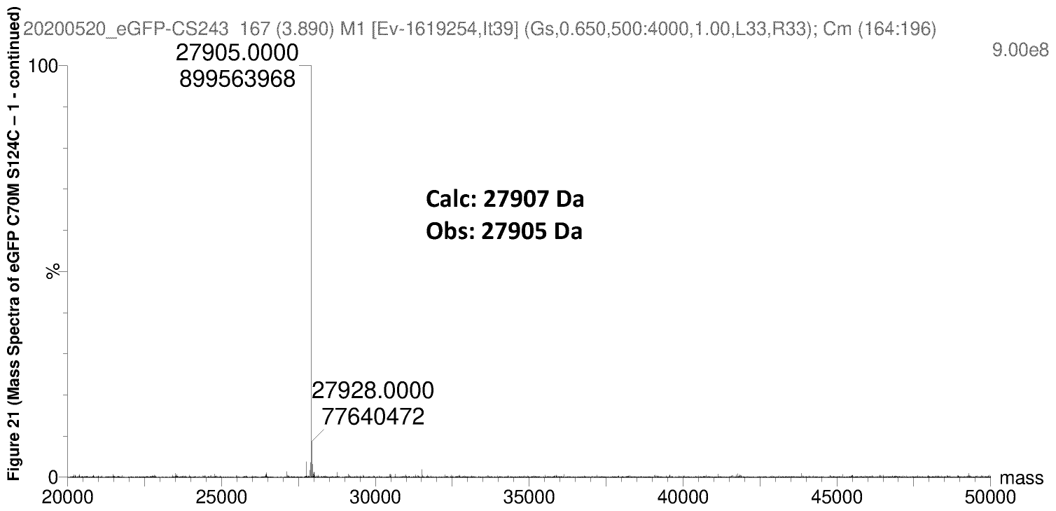

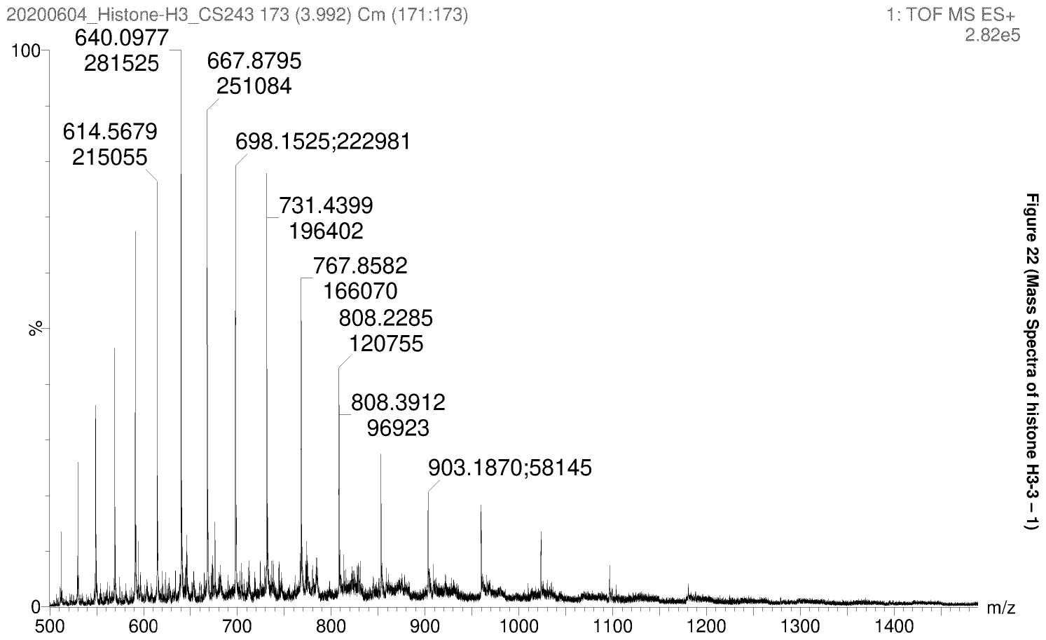

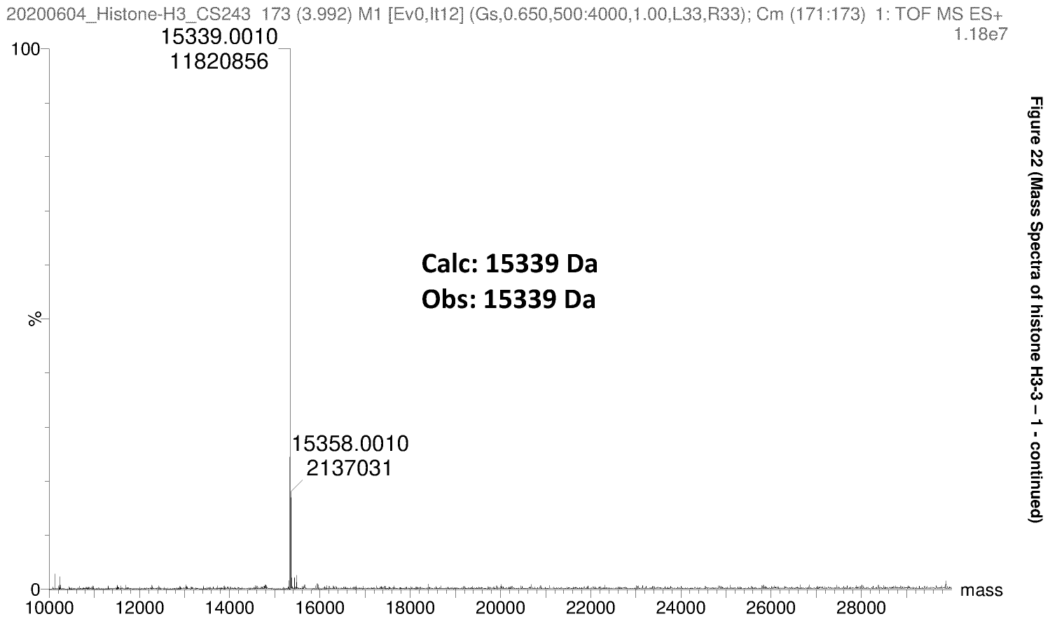

eGFP-1 was prepared according to section “Protein-modification with Bis-ethynyl-phosphinates” above using ethyl bis-ethynyl phosphinate 1 and with a protein-concentration of 100 μM. Mass spectra of eGFP C70M S124C-1 are shown in FIG. 21.

Histone H3-3-1 (in Accordance with Example 4)

Histone H3-3-1 was prepared according to section “Protein-modification with Bis-ethynyl-phosphinates” above using ethyl bis-ethynyl phosphinate 1 with a protein-concentration of 20 μM. Mass spectra of histone H3-3-1 are shown in FIG. 22.

Recombinant BSA—1 (in Accordance with Example 4)

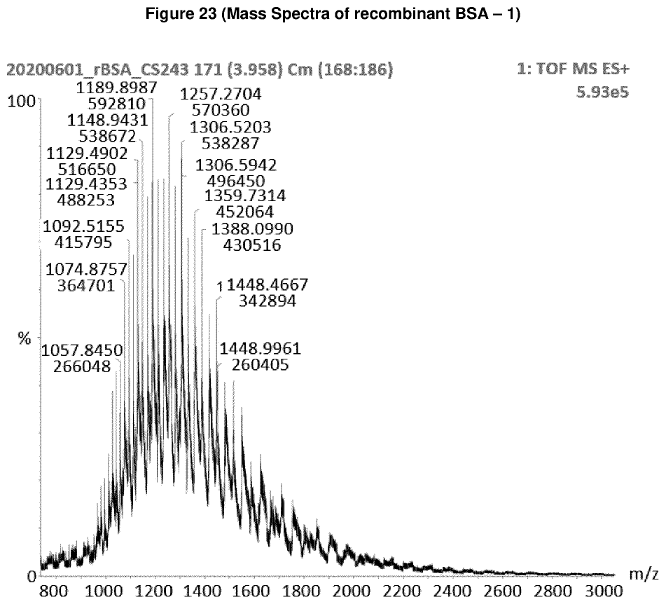

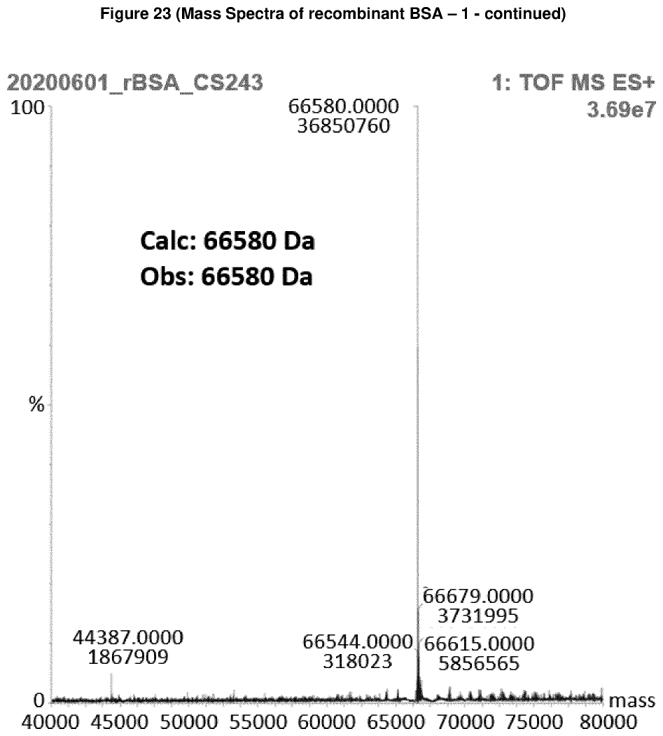

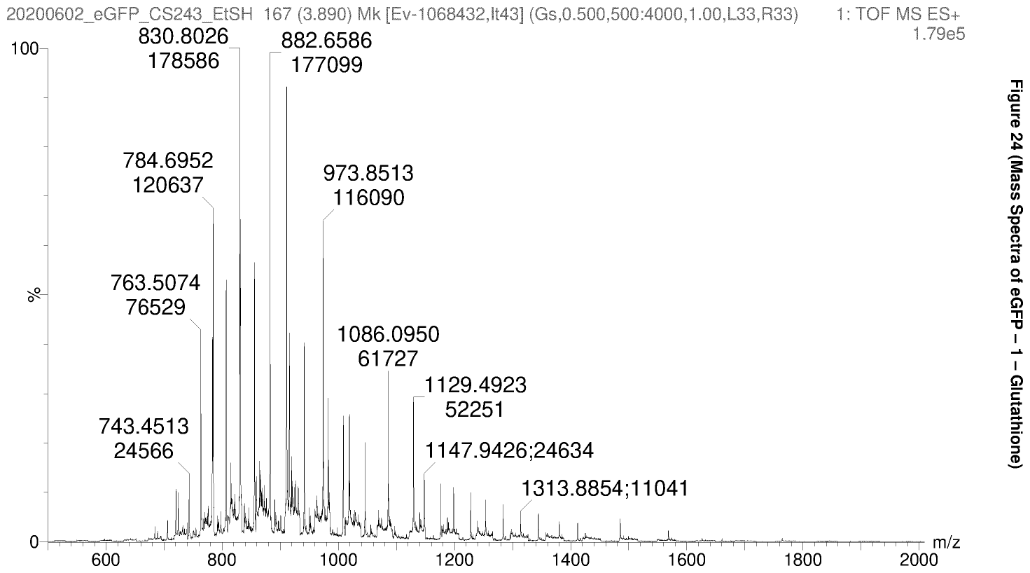

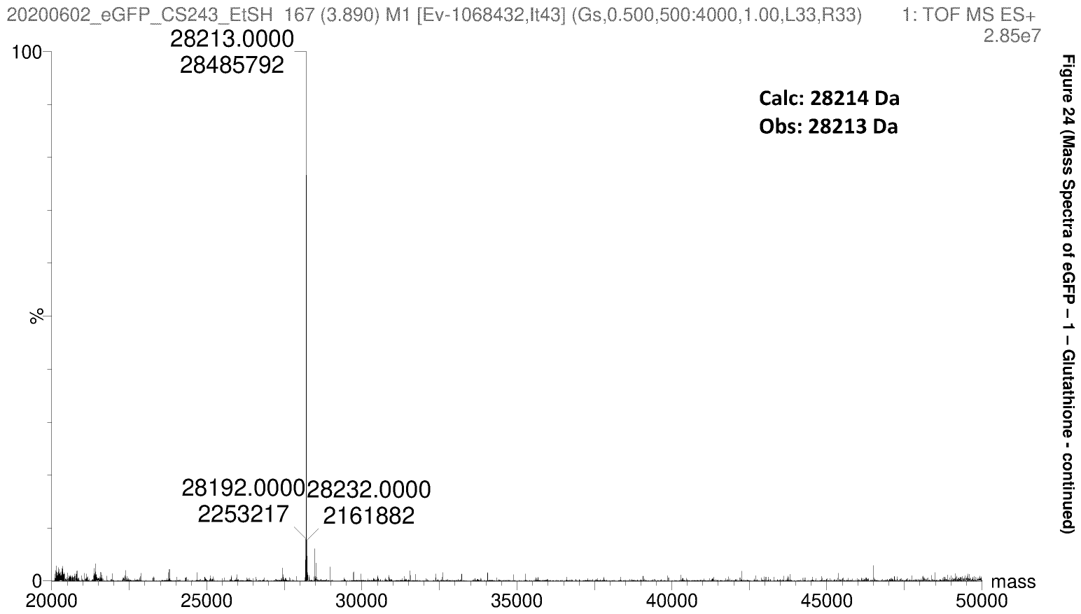

rBSA-1 was prepared according to section “Protein-modification with Bis-ethynyl-phosphinates” using ethyl bis-ethynyl phosphinate 1 with a protein-concentration of 10 μM. Mass spectra of rBSA-1 are shown in FIG. 23.

NLS-mCherry—5 (in Accordance with Example 4)

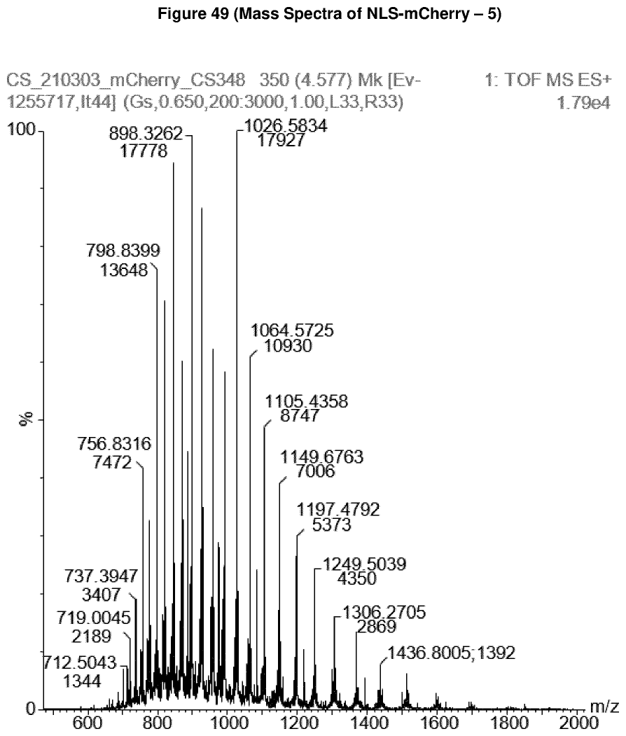

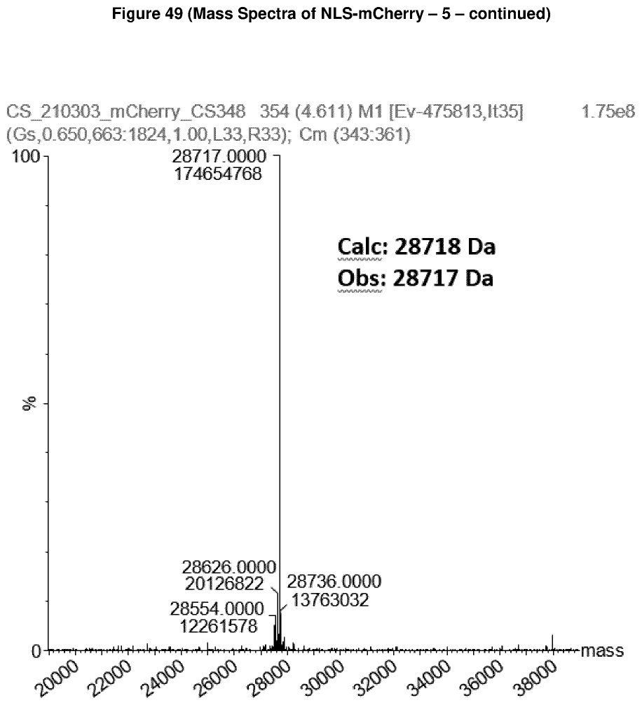

NLS-mCherry-5 was prepared according to section “Protein-modification with Bis-ethynyl-phosphinates” using NBD-bis-ethynyl phosphinate 5 with a protein-concentration of 120 μM. Mass spectra of NLS-mCherry-5 are shown in FIG. 49.

Thiol-Addition to Phosphinate-Modified eGFP (in Accordance with Example 6)

100 μl of phosphinate-modified eGFP (60 μM in PBS) were reacted with 20 eq. thiol for 16 h at room temperature. After the thiol-addition was completed, the excess reagents were removed by repeated diafiltration into PBS using an Amicon-Ultra centrifugal filter (10000 MWCO, Merck Millipore) and the modified protein was stored at −20° C. until further usage.

eGFP-1-Glutathione (in accordance with Example 6)

eGFP-1-Glutathione was Prepared According to Section “Thiol-Addition to phosphinate-modified eGFP” from eGFP-1 and glutathione as thiol. Mass spectra of the conjugate eGFP-1-Glutathione are shown in FIG. 24.

eGFP-1-R₁₀ (in accordance with Example 6)

eGFP-1-R₁₀ was prepared according to section “Thiol-addition to phosphinate-modified eGFP” from eGFP-1 and Cys-containing decaarginine

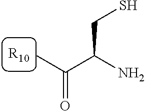

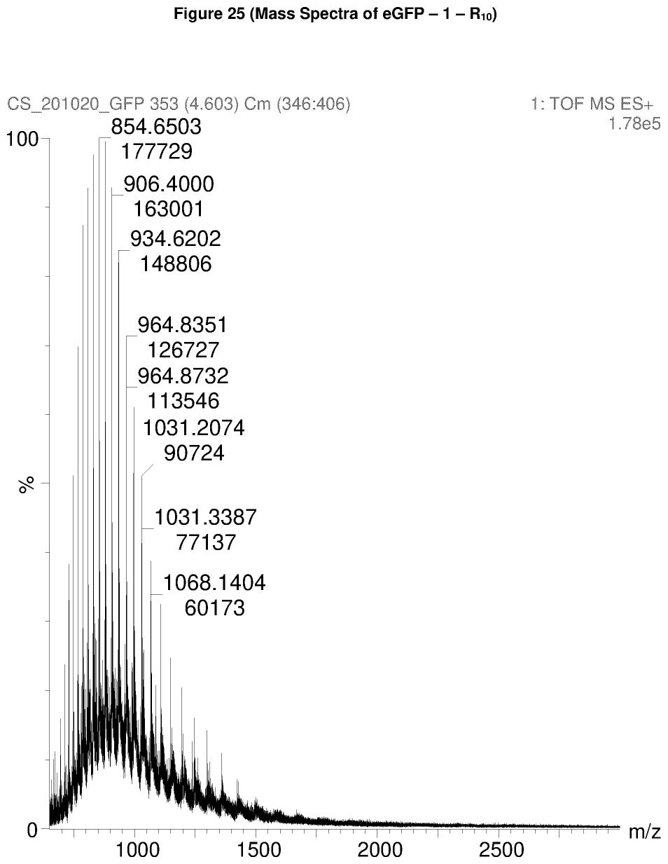

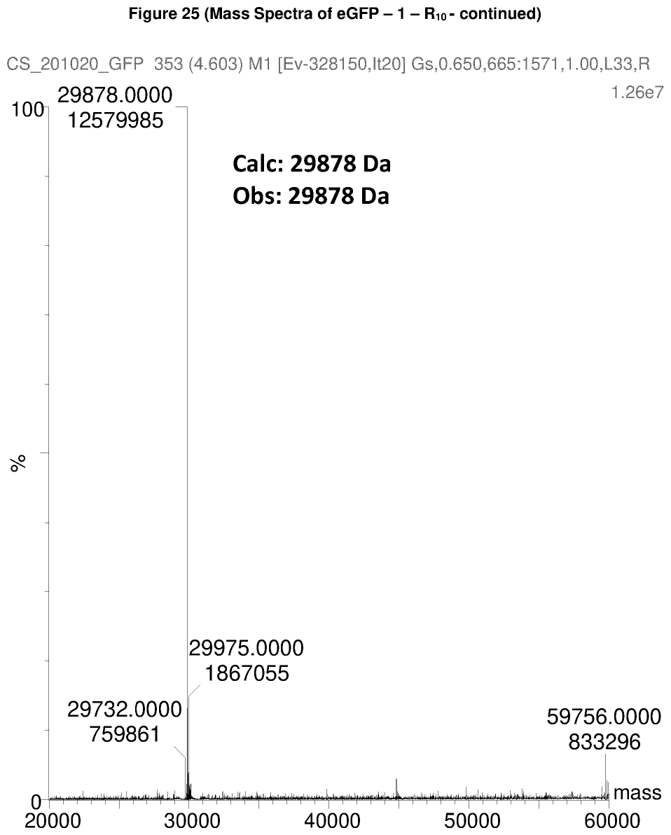

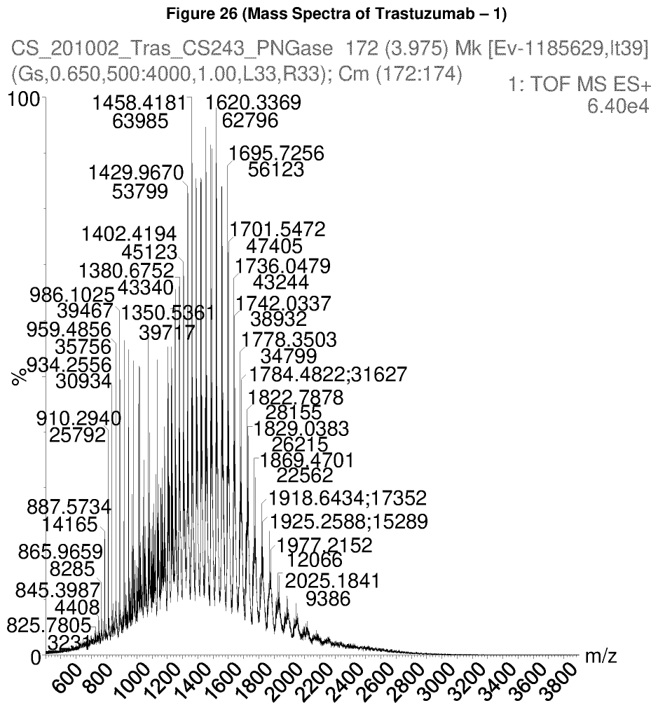

as thiol. Mass spectra of the conjugate eGFP-1-R₁₀ are shown in FIG. 25.
NLS-mCherry-Cys-5-R₁₀ (in accordance with Example 6)

NLS-mCherry-Cys-5-R₁₀ was prepared in an analogous manner to section “Thiol-addition to phosphinate-modified eGFP” from NLS-mCherry-Cys-5 and Cys-containing decaarginine

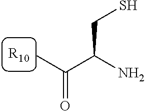

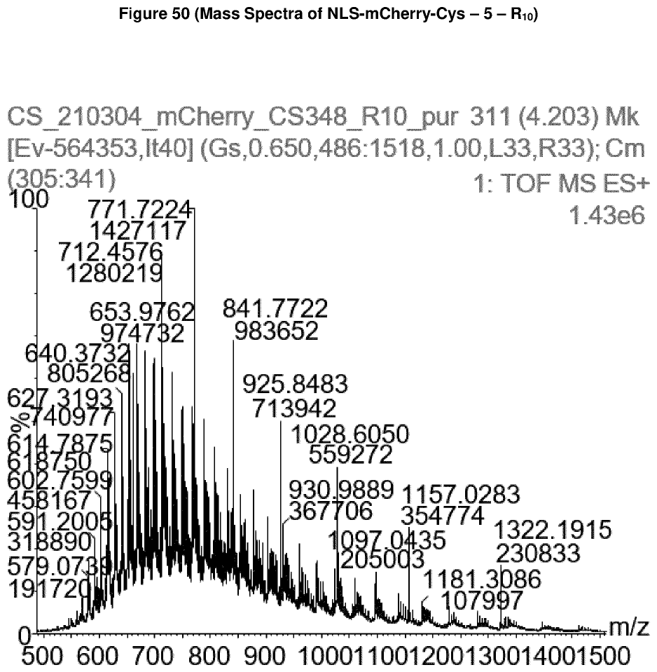

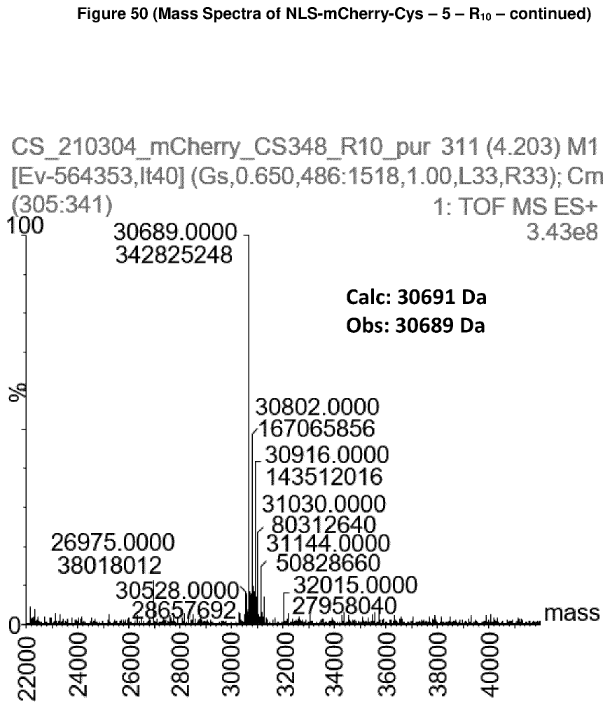

as thiol. Mass spectra of the conjugate NLS-mCherry-Cys-5-R₁₀ are shown in FIG. 50.
Cellular Uptake Experiments and Live-Cell Microscopy (in Accordance with Example 6)

Cells (HeLa Kyoto or CCL2) were grown at 37° C. in a humidified atmosphere with 5% CO₂ in DMEM 4.5 g/L glucose with 10% fetal bovine serum (FBS). 15′000 cells (30′000 for CCL2) were seeded per well of a 8-well ibidi μ-slide. Cells were left to adhere and grow for 24 hours at 37° C. and 5% CO₂. Cells were washed trice with Fluorobrite DMEM without FBS and incubated with 10 μM protein-R₁₀ and additives (10 μM TNB-R₁₀) in Fluorobrite DMEM without FBS. Cells were incubated for 1 h at 37° C. and 5% CO₂. Cells were washed three times with Fluorobrite DMEM with 20 mM glutamine and 10% FBS. The cells were then covered with Fluorobrite DMEM with 20 mM glutamine and 10% FBS containing Hoechst stain (Hoechst 33342).

Live cell microscopy images of the eGFP & mCherry uptake experiments were acquired on Nikon-CSU spinning disc microscope with a CSU-X1 (Andor) and live cell incubation chamber (OKOlab). All live cell images were acquired using a PlanApo 60× NA 1.4 oil objective (Nikon) and an EMCCD (AU888, Andor). Brightfield images were acquired along with fluorescence images. Standard laser, a quad Dicroic (400-410, 486-491, 560-570, 633-647, AHF) and Emission filters were used for the acquisition of confocal fluorescence images (BFP (Hoechst 33342) ex.: 405 nm em.: 450/50, GFP (GFP, NBD), ex.: 488 em.: 525/50, RFP (mCherry) ex.: 561 nm em.: 600/50).

General Procedure for Antibody Rebridging Using Phosphinates (in Accordance with Examples 7 and 11)

The interchain disulfides of the monoclonal antibody (5 mg/ml; 50 mM Tris, 1 mM EDTA, 300 mM NaCl, pH 8.4) were reduced using 10 eq. TCEP (37° C., 30 min). Subsequently, 5 eq. of phosphinate were added and the reaction was allowed to proceed overnight. The reaction was terminated by buffer-exchange to PBS using 0.5 mL Zeba™ Spin Desalting Columns with 7K MWCO (Thermo Fisher Scientific, USA). Re-bridging efficiency was evaluated via intact-protein mass spectrometry after deglycosylation with PNGaseF (see 3.6) and reducing SDS-PAGE. For the rebridging experiments using phosphinamidate II the same protocol was used, however varying amounts of II (5, 10, 20, 40, 100 eq.) were employed.

Deglycosylation, Reduction and MS-Analysis of Re-Bridged Trastuzumab (in Accordance with Example 7)

5 μl of the crude antibody modification mixture (5 mg/ml) were diluted to a final protein concentration of 1 mg/ml using PBS. 1 μl PNGase-F solution (Pomega, Germany, Recombinant, cloned from Elizabethkingia miricola 10 u/μl) and 2 μl TCEP solution (50 mM in H₂O) were added and the solution was incubated at 37° C. for >2 h and subjected to intact protein MS.

Trastuzumab-1 (in Accordance with Example 7)

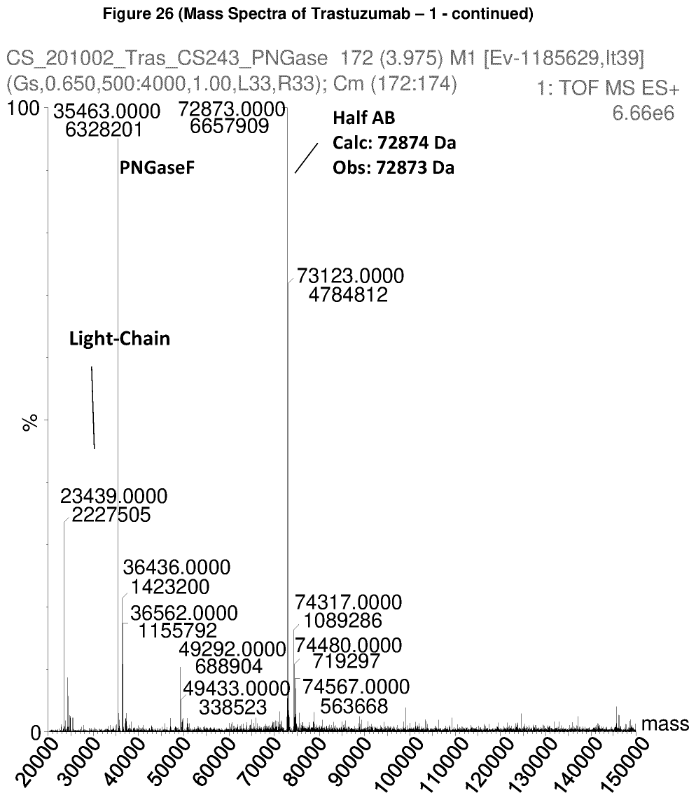

Trastuzumab-1 was prepared according to section “General procedure for Antibody rebridging using phosphinates” using Trastuzumab as antibody and ethyl bis-ethynyl phosphinate 1. Mass spectra of rebridged antibody Trastuzumab-1 were obtained according to section “Deglycosylation, reduction and MS-analysis of re-bridged Trastuzumab” and are shown in FIG. 26.

Trastuzumab-2 (in Accordance with Example 7)

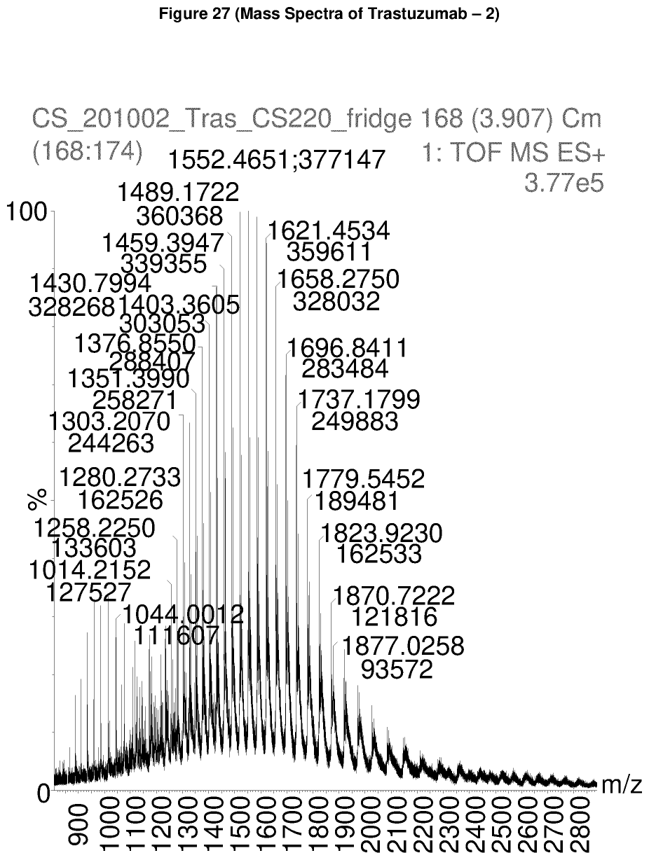

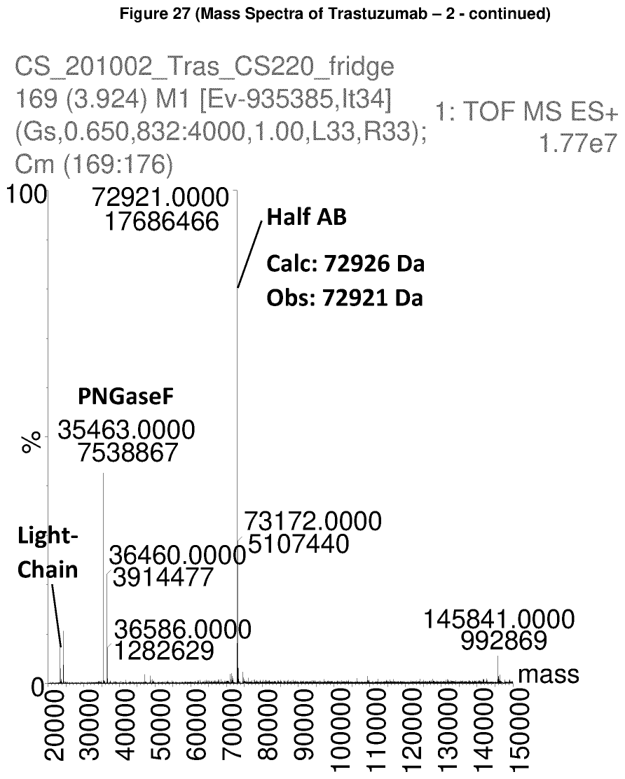

Trastuzumab-2 was prepared according to section “General procedure for Antibody rebridging using phosphinates” using Trastuzumab as antibody and but-3-yn-1-yl diethynylphosphinate 2. Mass spectra of rebridged antibody Trastuzumab-2 were obtained according to section “Deglycosylation, reduction and MS-analysis of re-bridged Trastuzumab” and are shown in FIG. 27.

Cu-Click modification of Trastuzumab-2 with fluorescein-Na (in accordance with Example 7)

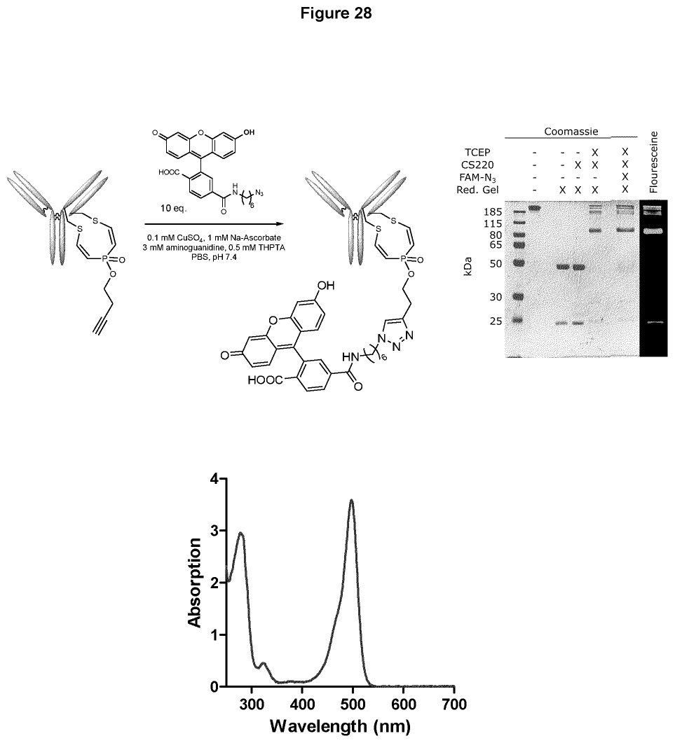

To a solution of Trastuzumab-2 (100 μL, 2 mg/mL) in PBS was added FAM-N₃ (5 mM in DMSO, to 200 μM), CuSO4·5H2O (to 300 μM), THPTA (to 0.5 mM) and sodium ascorbate (to 1.5 mM). The mixture was vortexed and incubated at r.t. for 2 h. The excess reagents were removed by repeated diafiltration into PBS using an Amicon-Ultra centrifugal filter (10000 MWCO, Merck Millipore). UV-vis analysis revealed conversion to an AFC with an average DAR of 4.03. FIG. 28 shows the click reaction of Trastuzumab-2 with FAM-N3, analysis of the conjugate via SDS-PAGE using Coomassie staining and in-gel fluorescence, and the UV-Vis spectrum of the fluorescein-conjugated antibody. The fluorophore to antibody ratio (FAR) was calculated according to the following formula:

### UV-Vis FAR Calculation:

PBS was used as a baseline for analysis.
FAR was calculated using the following formula:

$$F A R = \frac{{Abs}_{495} / {ε}_{495}}{( {Abs}_{280} - \times {Abs}_{495} ) / {ε}_{280}}$$

Abs₄₉₅=3.498; Abs₂₈₀=2.941; ε₂₈₀=215,380 M⁻¹ cm⁻¹ for Trastuzumab; ε₄₉₅=8030 M⁻¹ cm⁻¹ for fluorescein and a correction factor of 0.178 for fluorescein absorption at 280 nm.
Immunofluorescence of SK-BR-3 and MDA-MB-468 Cells with Labeled Trastuzumab (in Accordance with Example 7)

SKBR3 cells and MDA-MB-469 cells were grown in DMEM/Ham's F-12 with 10% FBS and 1% P/S at 37° C. in a humidified atmosphere with 5% CO₂. 30′000 SKBR3 or 15′000 MDA-MB-468 cells were seeded per well of an 8-well ibidi μ-slide respectively. Cells were left to adhere and grow overnight. Cells were washed three times with PBS and fixed for 10 minutes with 4% paraformaldehyde in PBS. The cells were then washed with 0.1% Triton X-100 in PBS (PBS-T). Antibody was added with 5 μg/mL in PBS and incubated 1 hour at room temperature in the dark. Cells were washed once with PBS-T and counterstained with Hoechst 33342. Cells were then imaged at a confocal laser scanning microscope.

Images were acquired with a Leica SP5II equipped with an ACS APO 63x/1.3 NA objective. Brightfield images were acquired along with fluorescent images. Argon-Laser (GFP, 488 nm) and Laserdiode CW (Hoechst 33342, 405 nm) and standard emission filters were used.

Equivalent Screen and Time-Course Experiments Using EDANS-Electrophiles and Trastuzumab (in Accordance with Example 8)

Trastuzumab (5 mg/ml, Tris-Buffer pH 8.5, 100 mM NaCl, 1 mM EDTA) was reduced with 8 eq. TCEP (30 min, 37° C.). Subsequently, X eq. electrophile were added (X=1, 2, 5, 6, 8, 10) and the reaction was allowed to proceed overnight at room temperature. The degree of labeling was determined by intact protein-MS after deglycosylation with PNGase-F (37° C., 1 mM TCEP, 1 h). For time-course experiments an aliquot was taken after 0.5, 1, 2, 4, 6 and 16 hours and analyzed via intact-protein MS.

Labelling of Trastuzumab Using Phosphinate CS375 (in Accordance with Example 10)

Trastuzumab (5 mg/ml, Tris-Buffer pH 8.5, 100 mM NaCl, 1 mM EDTA) was reduced with 8 eq. TCEP (30 min, 37° C.). Subsequently, 5 eq. phosphinate CS375 were added. After 4 h at room-temperature, intact protein-MS showed an average FAR of 4.5. Excess reagent was removed by spin-filtration (0.5 mL Zeba™ Spin Desalting Columns with 7K MWCO; Thermo Fisher Scientific, USA) and the protein was diluted to 1 mg/ml. Trastuzumab-CS375 was stored at 4° C. until further usage.

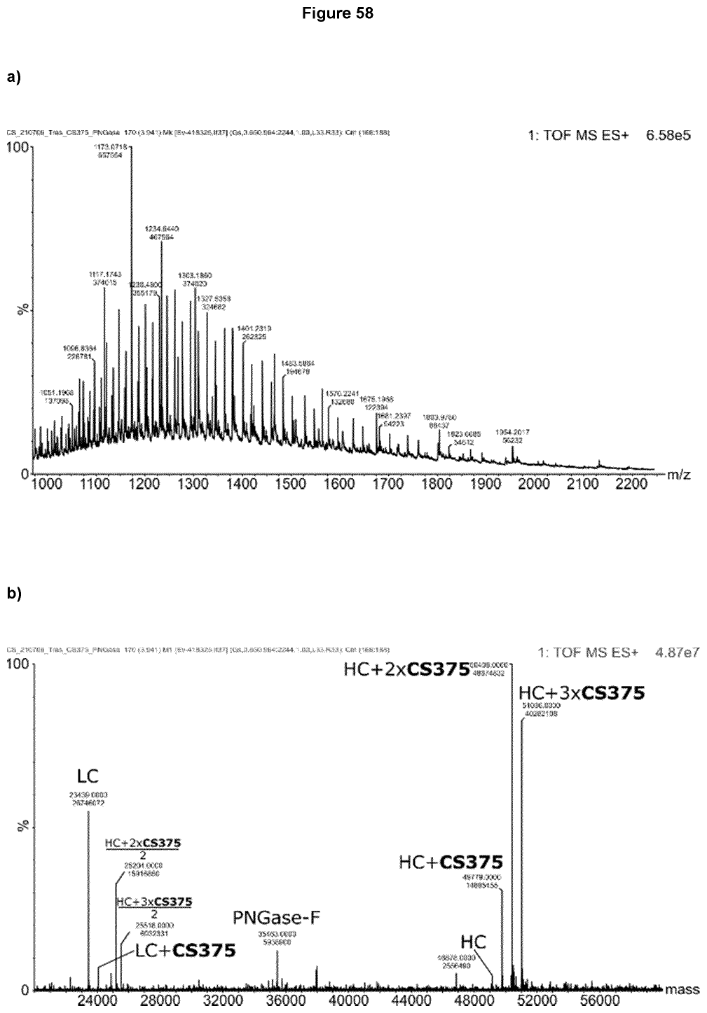

For analytical purposes, a small amount was deglycosylated using PNGaseF (37° C., 1 mM DTT, 1 h) and analyzed via intact-protein MS. FIG. 58 shows the mass spectra of the conjugate Trastuzumab-CS375 obtained by intact-protein MS. FIG. 58a shows the non-deconvoluted MS-spectrum. FIG. 58b shows the deconvoluted MS-spectrum.

Labelling of Trastuzumab with ETP-Modified Cytotoxic Payload (in Accordance with Example 13)

Trastuzumab (5 mg/ml, Tris-Buffer pH 8.5, 100 mM NaCl, 1 mM EDTA) was reduced with 8 eq. TCEP (30 min, 37° C.). Subsequently, 5 eq. MMAE-phosphinate CSDrug1 were added. After 4 h the reaction mixture was purified by size-exclusion chromatography with a 25 ml Superose™ 6 Increase 10/300GL (GE healthcare, United States) and a flow of 0.75 ml/min eluting with sterile PBS (Merck, Germany). The antibody containing fractions were pooled and the final concentration was determined (see Example 13 under protein concentration determination). For DAR analysis, 10 μl of this sample were analyzed using hydrophobic interaction chromatography. Trastuzumab-CSDrug1 was stored at 4° C. until further usage.

FIG. 51b shows an HIC-chromatogram of Trastuzumab-CSDrug1.

FIG. 51e shows an intact-protein MS of the crude reaction mixture of Trastuzumab-CSDrug1.

Cell Based Anti Proliferation Assays (in Accordance with Example 13)

SKBR3 and MDA-MB-468 cell lines were cultured in DMEM/F12 medium supplemented with 10% FCS and 0.5% Penicillin-Streptomycin. Cells were seeded (100 μL) at a density of 5*10³ cells/well (SKBR3) or 2*10³ cells/well (MDA-MB-468) in 96-well cell culture microplates. Plates were incubated for 24 h at 37° C., 5% CO₂. Subsequently, the cells were aspirated and respective wells on the microplate were directly subjected to 1:4 serial dilutions of ADCs/antibodies in medium (5% PBS; 100 μL) starting at 62.5 nM final concentration. Plates were incubated for 96 h at 37° C., 5% CO₂. Subsequently, the cells were aspirated and resazurin (100 μM in medium; 100 μL) was added, followed by incubation for 4 h at 37° C., 5% CO₂. Metabolic conversion of resazurin to resorufin was quantified by the fluorescent signal of resorufin (λ_{EX}=560 nm, λ_{EM}=590 nm, excitation bandwidth=9 nm, emission bandwidth=20 nm, gain (manual)=50, number of flashes=25, integration time=20 μs, lag time=0 ρs, settle time=0 ms, Z-position (manual)=20000 μm) on a Tecan Infinite 200 Pro microplate reader. Data analysis was performed with Graphpad Prism 5 software. Raw data was normalized to 0% viability (cells treated with 10 μM MMAE in medium with 5% PBS; 100 μL) and 100% viability (cells treated with medium with 5% PBS; 100 μL). Mean and standard error of the mean (SEM) were calculated from three biological replicates which all contained triplicate datasets (N=3, n=3) and plotted against ADC/antibody concentration. IC₅₀ values were calculated using a nonlinear regression (log inhibitor vs response).

Expression of UbK48Aha and UbK63Aha (in Accordance with Example 14)

The ubiquitin mutant DNA-sequence in a pET28a vector containing an N-terminal His6-tag and a thrombin cleavage site was used. This plasmid together with T7 polymerase containing pTARA vector was transformed into auxotrophic E. coli cell line B834. One colony from the LB-Agar plate containing 30 μg/mL Kanamycin (Fa. Roth #T832.3) and 34 μg/mL Chloramphenicol (Fa. Roth #388.2) was picked for the overnight culture. To start with OD600 of 0.05 5-10 mL of overnight culture were added to 500 mL NMM medium containing 45 μM methionine as limited amino acid, 0.75% arabinose, 30 μg/mL kanamycin and 34 μg/mL chloramphenicol. The cells were growing at 28° C. and 180 rpm overnight until the stationary phase was reached. Then azidohomoalanine (100 mg/L) was added and the cells were incubated for 30 min at 37° C. Subsequently, the expression was induced by adding 1 mM IPTG (Fa. Thermo Scientific, #R0392) and was shaken at 28° C. for 16-18 h at 180 rpm. The cells were harvested by centrifugation at 4000 RCF for 15 min at 4° C., washed with 1×PBS pH 7.4, centrifuged again and resuspended in 30 mL lysis buffer (PBS pH 7.4, 10 mM imidazole). The Lysis was performed by microfluidizer. The lysate was centrifuged at 50000 RCF for 15 min at 4° C. and the supernatant was purified by HisTrap (Cytiva, 17-5255-01) with lysis buffer as binding buffer. The protein was eluted with 1×PBS pH 7.4 containing 500 mM imidazole. After desalting with HiPrep 26/10 desalting column (GE, #17-5087-01) in 20 mM Tris pH 8, 150 mM NaCl, 2.5 mM CaCl2, the His6-tag was cleaved with thrombin (1 U/mL) (Bovine thrombin, Fa. Merck Millipore #605157) at rt overnight. The cleaved protein was purified by HisTrap in 1×PBS pH 7.4, 10 mM imidazole as binding buffer to remove remained His6-taged protein. The flow-through fraction was collected and the protein concentration was determined. The expressed protein was isolated in 2-10 mg yield for 1 L expressions.

ETP-functionalisation of UbK48Aha and UbK63Aha (in Accordance with Example 14)

UbK48/63Aha mutants were diluted to 1 mg/ml (0.115 mM) in PBS pH 6.9. The reaction was started by adding phosphinate 1 to a final concentration of 2.3 mM (20 eq.) and a premixed solution of CuBr (50 mM), THPTA (50 mM) to a final concentration of 2.5 mM (final MeCN concentration 5%). After 5 h reaction time 5 mM EDTA was added to terminate the reaction. Subsequently, the reaction mixture was dialyzed against the 1000-fold volume PBS (pH 6.9, 5 mM EDTA) overnight. Phosphinate modified Ubiquitins were further polished via SEC on an Äkta FPLC system equipped with a Superdex 75 10/300 GL column (GE Healthcare, USA) and a flow of 0.75 ml/min eluting with sterile PBS (Merck, Germany). Protein containing fractions were pooled, concentrated to a concentration of 200 μM and stored at 4° C. until further usage.

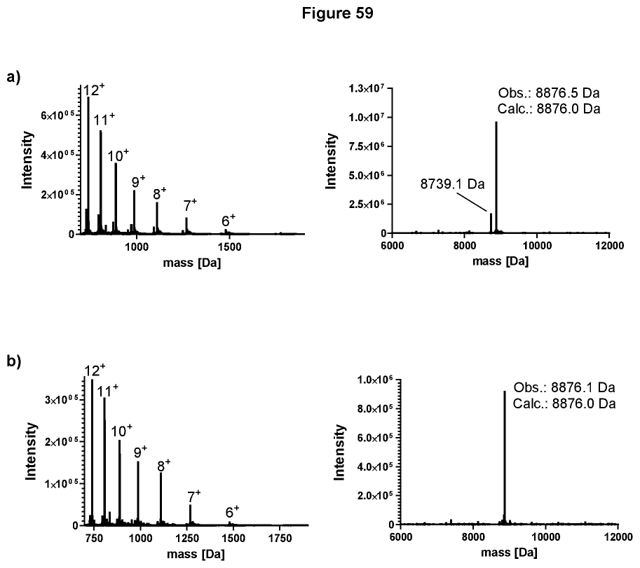

FIG. 59 shows the intact-protein mass spectra of UBK48-ETP and UbK63-ETP. FIG. 59a shows the intact-protein MS of UBK48-EPT. The peak with a mass of 8739.1 Da corresponds to UbK48M, an impurity from auxotrophic expression. FIG. 59b shows the intact-protein MS of UbK63-ETP.

Protein-Protein Cross-Linking Using ETP-Functionalized Ubiquitin (in Accordance with Example 14)

To 0.2 ml of UbG76C (0.5 mM) in Tris-EDTA buffer (50 mM Tris, 150 mM NaCl, 5 mM EDTA, pH 8.3) was added TCEP (1.05 eq.) and the mixture was shaken for 30 min at 37° C. After the initial reduction, 0.2 eq. of UbK63/48-ETP were added and the reaction was allowed to proceed for 6 h. Thereafter, the crude was purified by SEC on an Äkta FPLC system equipped with a Superdex 75 10/300 GL column (GE Healthcare, USA) and a flow of 0.75 ml/min eluting with sterile PBS (Merck, Germany). Product containing fractions were pooled and stored at −20° C. until further usage.

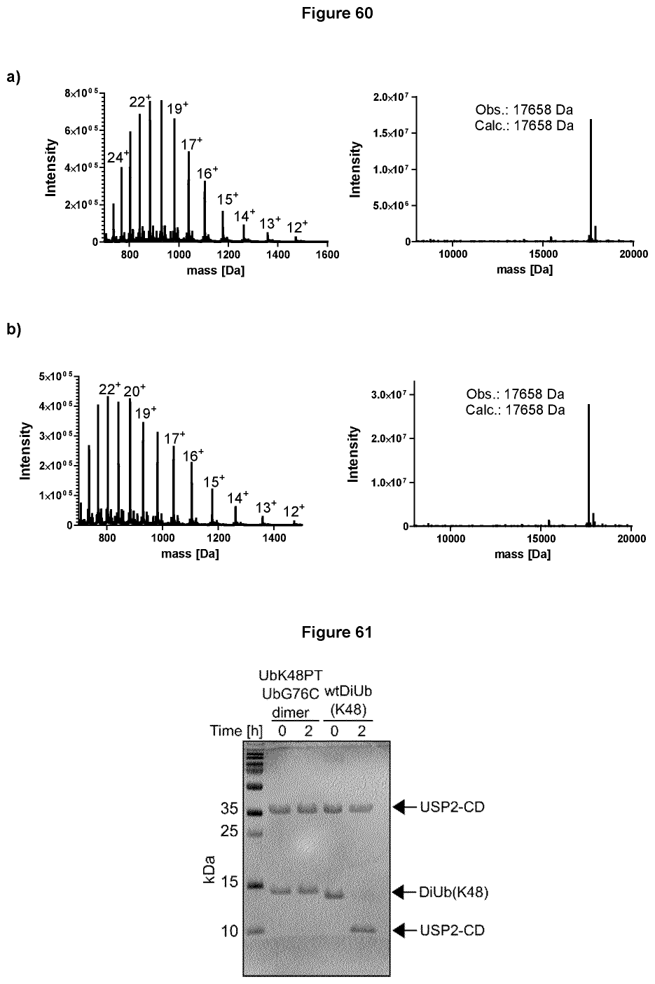

FIG. 60 shows intact-protein MS of the UbK48ETP-UbG76C dimer 12 and the UbK63ETP-UbG76C dimer 13. FIG. 60a shows intact-protein MS of the UbK48ETP-UbG76C dimer 12. FIG. 60b shows intact-protein MS of the UbK63ETP-UbG76C dimer 13.

Hydrolytic Stability of 12 Against USP2-CD (in Accordance with Example 14)

To the deubiquitinating enzyme (DUB) USP2-CD (1.6 μg, 40 pmol) (BostonBiochem) in 50 mM HEPES pH 8.0 containing 150 mM NaCl, 0.1 mM EDTA and 1 mM DTT was added either the 12 (1 μg) or wtDiUb(K48) (1 μg) (Enzo Life Sciences) in PBS pH 7.4 and the mixtures were incubated for 2 h at 37° C.

FIG. 61 shows the SDS-PAGE analysis of dimer 12 incubated with USP2-CD.

Tryptic Digest of Artificial Diubiquitins (in Accordance with Example 14)

Ubiquitin dimers (1 mg/ml, 50 mM HEPES pH 7.4) were supplemented with 1.5% (wt/vol) sodium deoxycholate (SDC) and heated to 60° C. for 30 minutes. The solution was diluted to 1% (wt/vol) SDC and trypsin was added 1:20 (wt/wt). The protein was digested for 4 h at 37° C. and the reaction was stopped by adding 1% TFA. Precipitated SDC was removed by centrifugation and the supernatant was stored at −20° C. until analysis.

Proteome-Wide Cysteine Labelling in HEK293-Cell Lysate Using Phosphinate CS375 (in Accordance with Example 15)

HEK293 cells were grown in 75 cm² cell culture flasks to ca. 80% of the total area. The cells have been washed by PBS and lysed in 50 mM Tris pH 7.5 containing 150 mM NaCl, 5 mM EDTA, 1 mM PMSF and 0.5% Triton x-100 by centrifugation. The total amount of the protein was detected by BCA assay and was adjusted to 1 mg/ml with the PBS. Cell lysate was reacted with increasing concentrations of phosphinate CS375 (0, 30, 75, 150, 300 μM) for 45 minutes at room-temperature. The labelled lysate was subsequently analyzed by SDS-PAGE by using in-gel fluorescence and coomassie staining.

Sample Preparation for Proteome-Wide Cysteine Profiling (in Accordance with Example 15)

For proteome-wide cysteine profiling using DTB-phosphinate CS418 the workflow described by Zanon et al. (Zanon, P. R. A. et al. Profiling the proteome-wide selectivity of diverse electrophiles. https://fragpipe.nesvilab.org (2021) doi:10.26434/CHEMRXIV.14186561.V1) was adapted.

HEK293 cells were grown in 75 cm² cell culture flasks to ca. 80% of the total area. The cells have been washed by PBS and lysed in 50 mM Tris pH 7.5 containing 150 mM NaCl, 5 mM EDTA, 1 mM PMSF and 0.5% Triton x-100 by centrifugation. The total amount of the protein was detected by BCA assay and was adjusted to 1 mg/ml with PBS. Cell lysate was reacted with 200 μM CS418 for 45 minutes at room-temperature. Subsequently, proteins were precipitated in 2 ml ice-cold acetone overnight at −20° C. Precipitates were centrifuged at 3,500 rpm at 25° C. for 10 min and resuspended in 0.5 ml cold MeOH. The suspension was centrifuged at 10′000×g for 5 minutes and the supernatant was removed. This procedure was repeated two more times. Proteins were resuspended in 150 μL 8 M urea in 100 mM triethylammonium bicarbonate (TEAB, Sigma Aldrich) and subsequently diluted to 2 M urea using 100 mM TEAB buffer. This solution was added to pre washed high capacity Streptavidin-beads (25 μl initial slurry) and incubated for 1 h at room-temperature with gentle agitation.

To remove unbound proteins, beads were centrifuged and the supernatant was discarded. Additionally, the beads were washed 3× with 300 μl PBS supplemented with 0.1% NP-40, 3× with 300 μl PBS and with 300 μl milliQ-water. Beads were resuspended (8M Urea, 100 mM TEAB) and treated with 15 μl of dithiothreitol (DTT; 31 mg/mL in water) at 37° C. for 45 minutes. Reduced cysteines were alkylated with 15 μl of iodoacetamide (74 mg/mL in water) for 45 minutes in the dark. After centrifugation and removal of the supernatant, the beads were resuspended in 0.5 ml TEAB-buffer (100 mM, 2 M Urea) and 1 μg trypsin was added (Promega, V5113). Proteins were digested at 37° C. overnight.

The supernatant, containing non-labeled peptides was discarded and the beads were washed 3× with 300 μl PBS supplemented with 0.1% NP-40, 3× with 300 μl PBS and with 300 μl milliQ-water. Desthio-biotinylated peptides were eluted two times with 100 μl 50:50:0.1 water/MeCN/FA. Solvent was removed by lyophilization, the samples were redissolved in 30 μl 5:95:0.1 water/MeCN/FA and stored at −20° C. until measurement.

### Example 18: Organic and Peptide Synthesis

Ethyl diethynyl phosphinate (1)

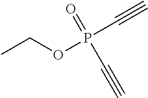

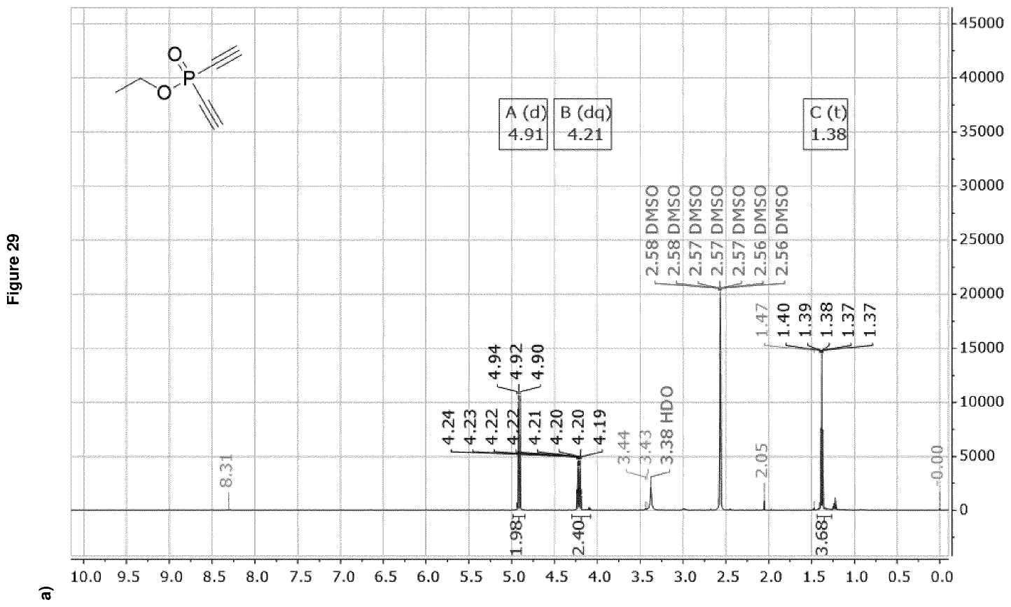

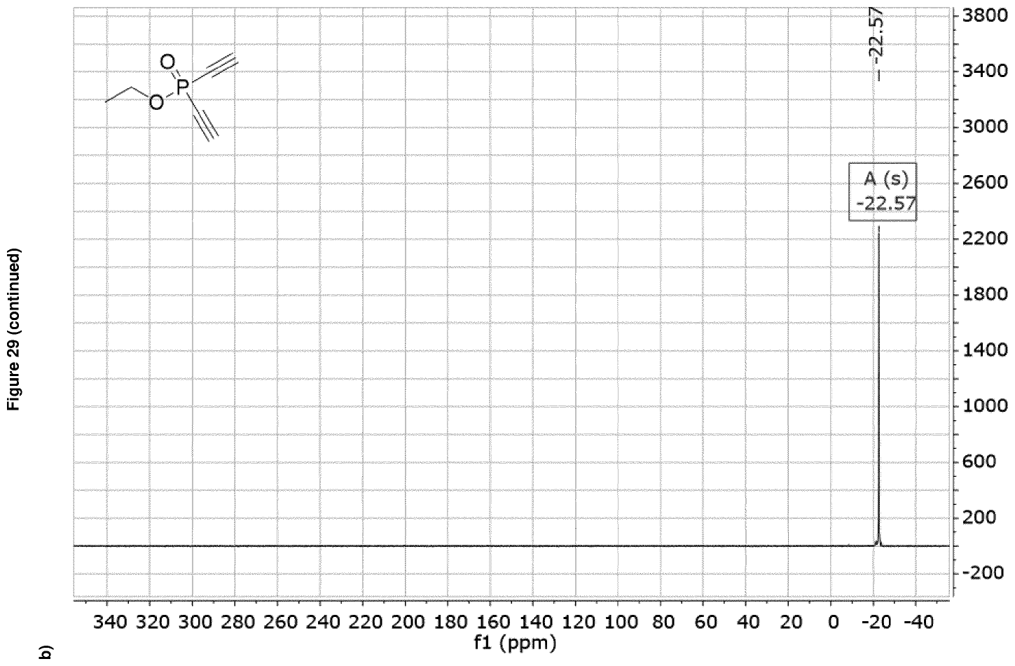

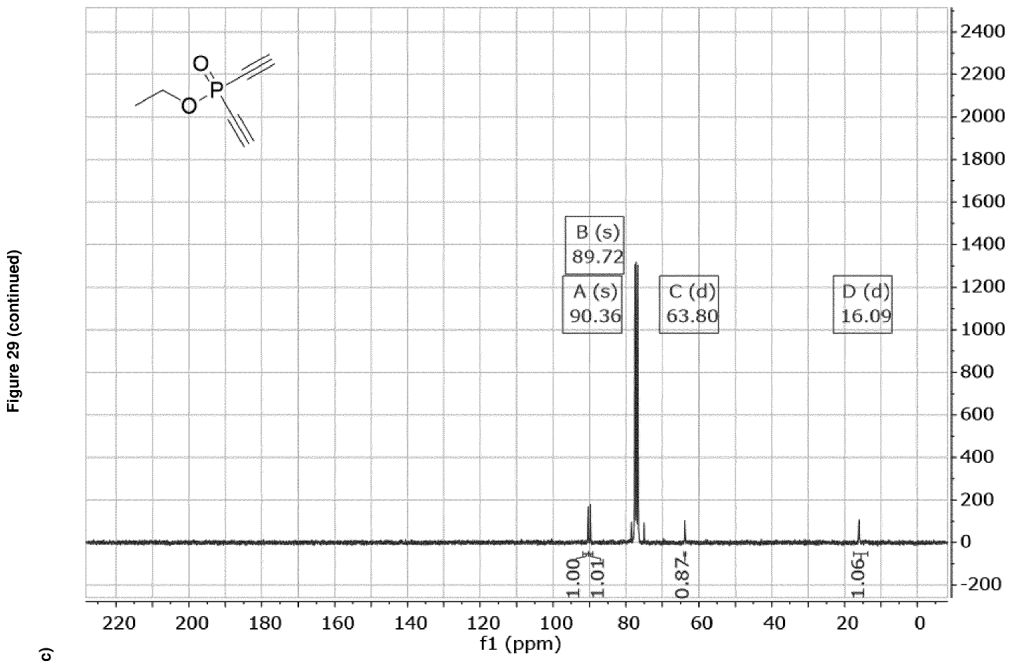

A 50 ml Schlenk-flask was charged with 115 μl ethyl-dichlorophosphite (1 mmol, 1 eq.) dissolved in 2 ml of dry THF under argon. After cooling to −78° C., 2.2 eq. ethynyl-magnesiumbromide (0.5 M in THF, 22 ml) were added dropwise. After the reaction was allowed to warm to room temperature, the mixture was poured onto 40 ml ice-cold H₂O containing 20 mmol H₂O₂. The solution was extracted 3× with 40 ml DCM, combined organic layers were washed twice with 50 ml H₂O and dried over MgSO₄. After solvent evaporation, 1 was obtained as a red liquid. (100 mg, 70% yield). NMR spectra of 1 are shown in FIG. 29 (a)¹H; b)³¹P; c)¹³C).

¹H NMR (600 MHz, DMSO-d₆) δ4.91 (d, J=12.8 Hz, 2H),4.21 (dq, J=9,9, 7.0 Hz, 2H),1.38 (t, J=7.0 Hz, 3H).

¹³C NMR (75 MHz, CDCl₃) δ90.36 (2C),89.72 (2C),63.80 (d, J=4.1 Hz),16.09 (d, J=7.9 Hz).

³¹P-NMR (122 MHz, CDCl₃) δ−22.57.

HRMS for C₆H₈O₂P⁺[M+H]⁺ calc.:143.0256; found:143.0257

Ethyl bis(2-(ethylthio)vinyl)phosohinate (5)

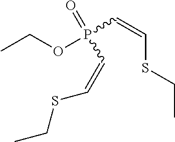

A 10 ml flask was charged with 72 mg 1 (0.5 mmol, 1 eq.) dissolved in 1 ml DMF. 150 μl ethanethiol (2 eq., dissolved in 1 ml DMF) were added dropwise and the reaction was stirred for 10 min at room temperature. The reaction was quenched with 2 ml 1% TFA in H₂O and different E/Z—isomers were separated via semi preparative HPLC.

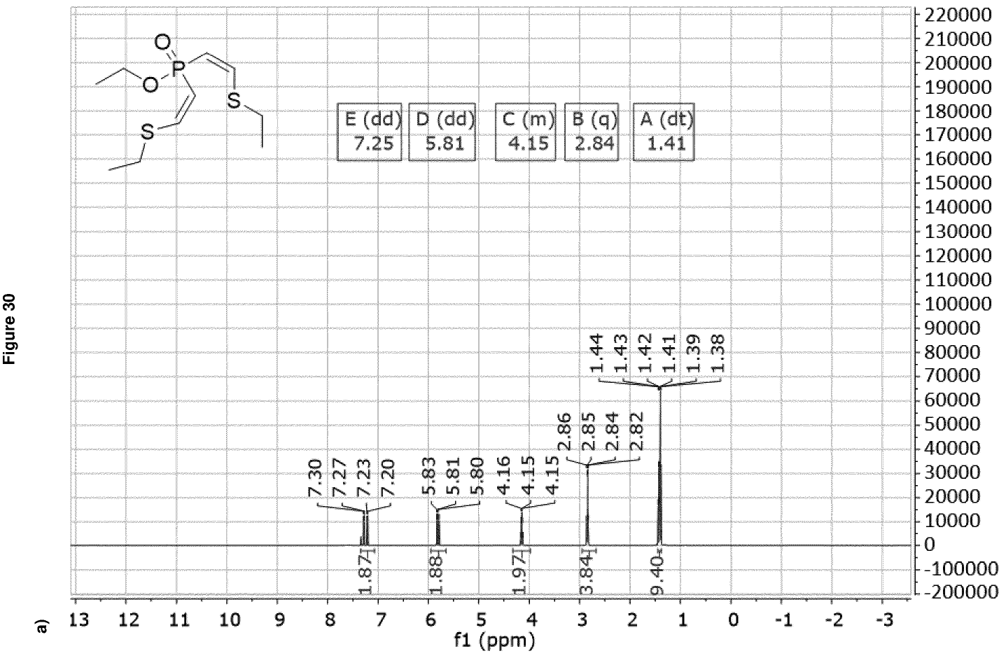

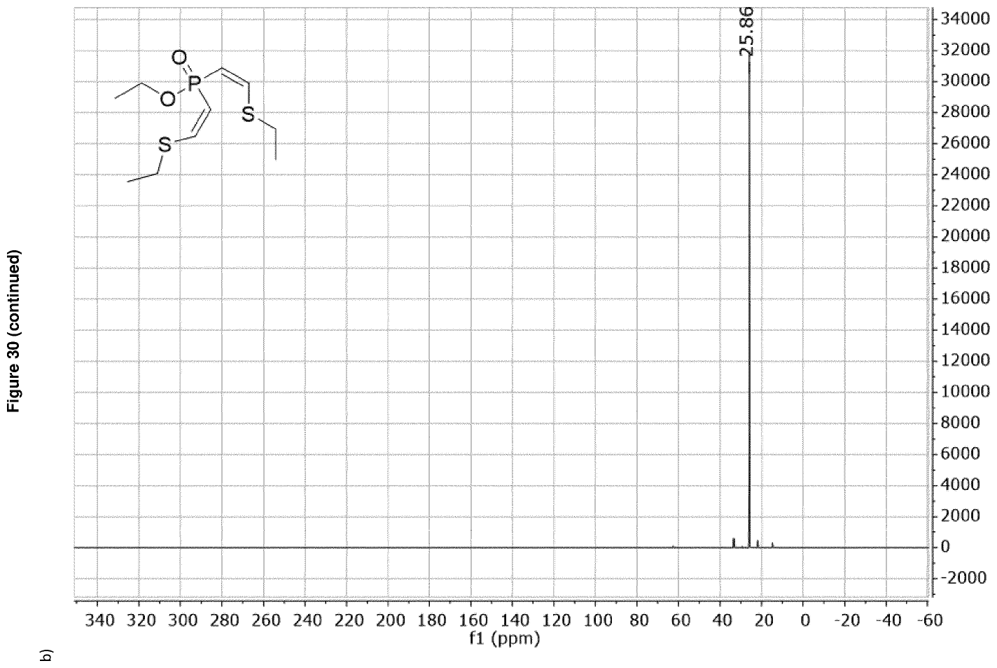

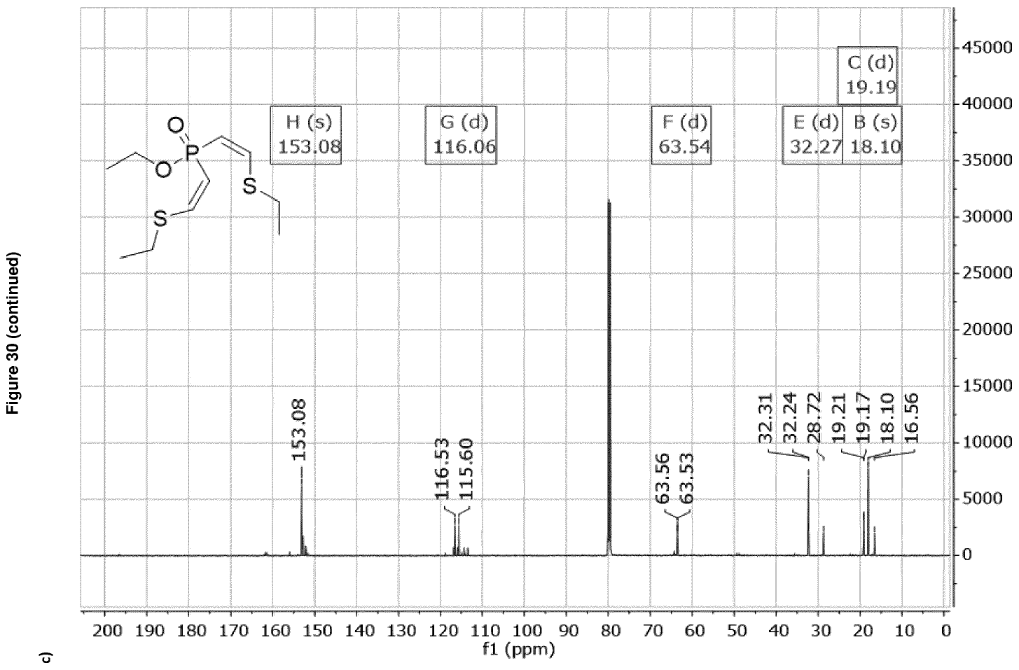

29 mg of the ZIZ-Isomer were obtained as a light yellow liquid. NMR spectra of the Z/Z-isomer are shown in FIG. 30 (a)¹H; b)³¹P; c)¹³C).

¹H NMR (600 MHz, CDCl₃) δ 7.25 (dd, J=42.2, 12.4 Hz, 2H), 5.81 (dd, J=16.4, 12.4 Hz, 2H), 4.32-3.97 (m, 2H), 2.84 (q, J=7.4 Hz, 4H), 1.41 (dt, J=20.4, 7.3 Hz, 9H).

¹³C NMR (151 MHz, CDCl₃) δ 153.08 (2C), 116.06 (d, J=139.9 Hz, 2C), 63.54 (d, J=5.2 Hz), 32.27 (d, J=9.4 Hz, 2C), 19.19 (d, J=6.9 Hz), 18.10 (2C).

³¹P-NMR (243 MHz, CDCl₃) δ 25.86.

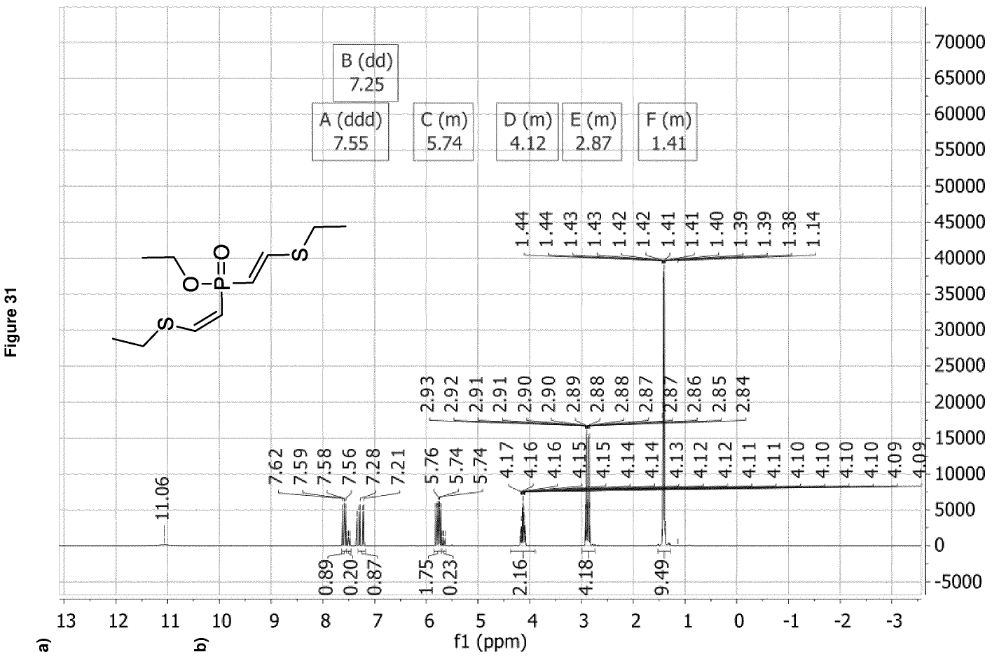

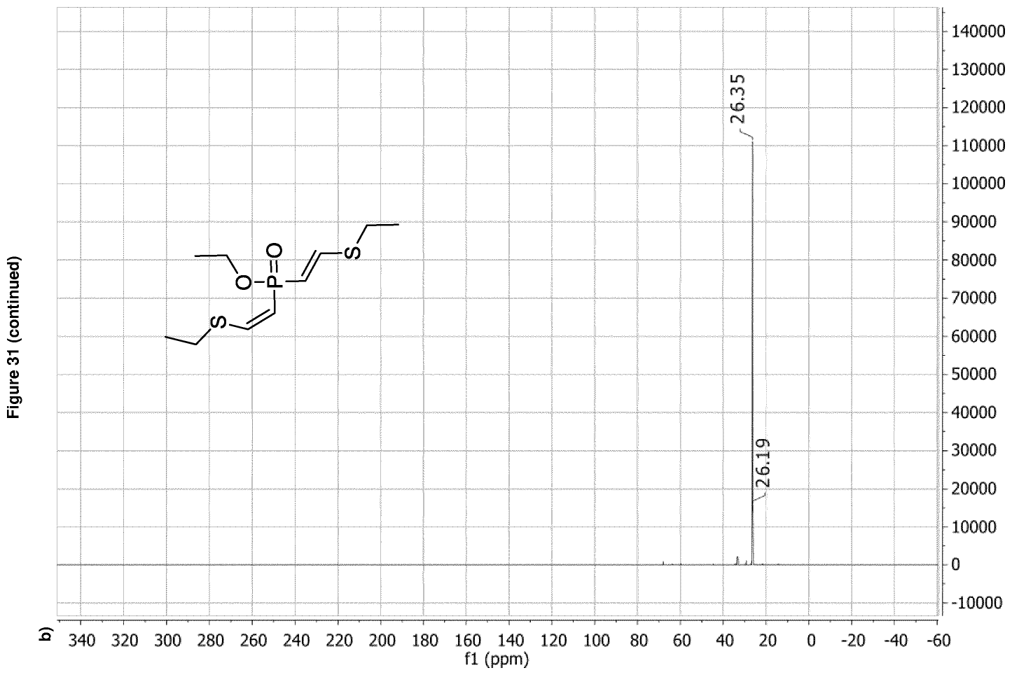

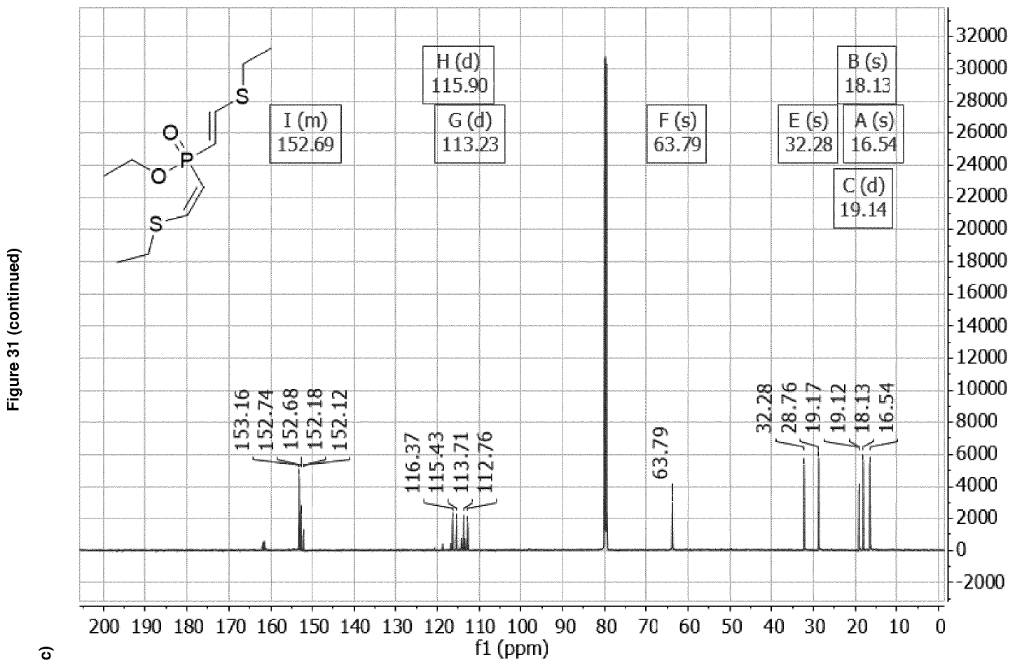

HRMS for C₁₀H₂₀O₂PS2⁺ [M+H]⁺ calc.: 267.0637; found: 267.0645. 32 mg of the E/Z-Isomer were obtained as an orange liquid. NMR spectra of the E/Z isomer are shown in FIG. 31 (a)¹H; b)³¹P; C)¹³C).

¹H NMR (600 MHz, CDCl₃) δ 7.59 (dd, J=20.4, 16.6 Hz, 1H), 7.25 (dd, J=42.8, 12.5 Hz, 1H), 6.02-5.61 (m, 2H), 4.45-3.81 (m, 2H), 2.88 (dq, J=27.0, 7.4 Hz, 4H), 1.67-1.04 (m, 9H).

¹³C NMR (151 MHz CDCl₃) δ 153.32-152.04 (m), 115.90 (d, J=142.1 Hz), 113.23 (d, J=143.0 Hz), 63.79, 32.28, 28.76, 19.14 (d, J=6.8 Hz), 18.13, 16.54.

³¹P-NMR (243 MHz, CDCl₃) δ 26.35.

HRMS for C₁₀H₂₀O₂PS2⁺ [M+H]⁺ calc.: 267.0637; found: 267.0645.

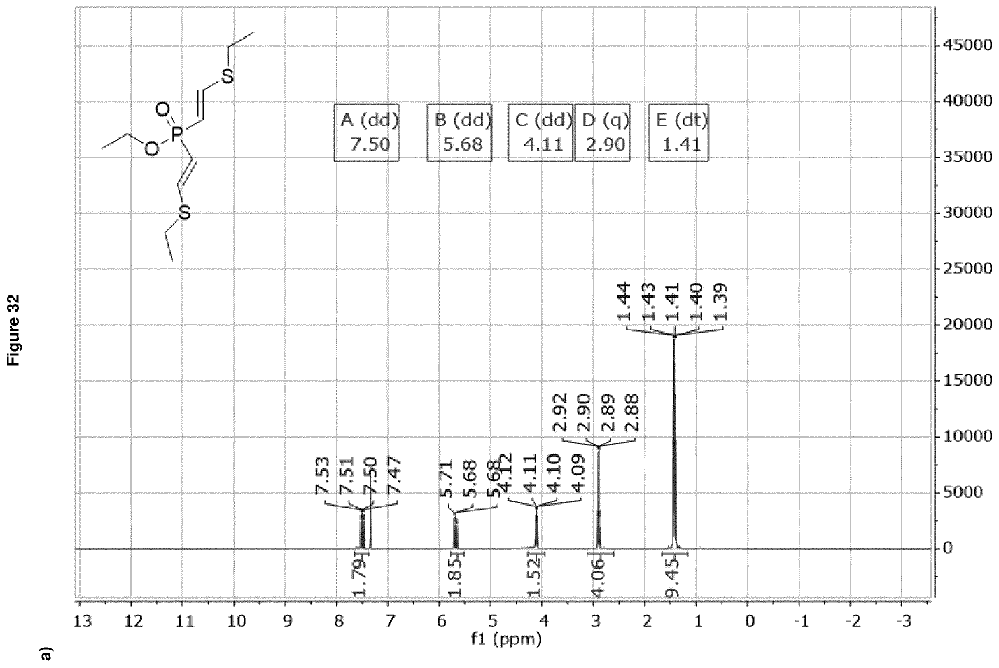

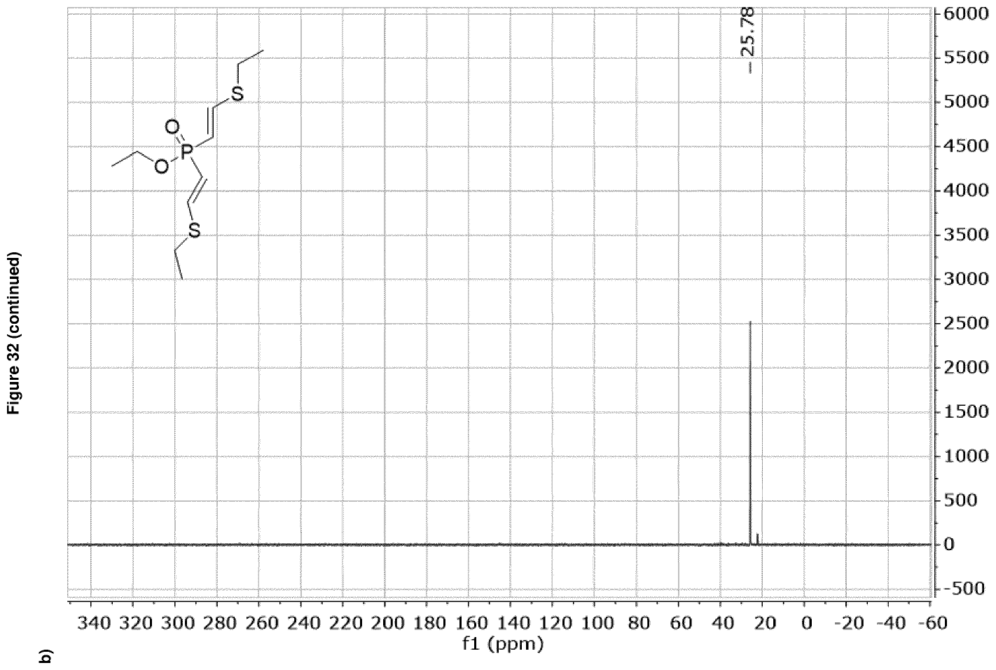

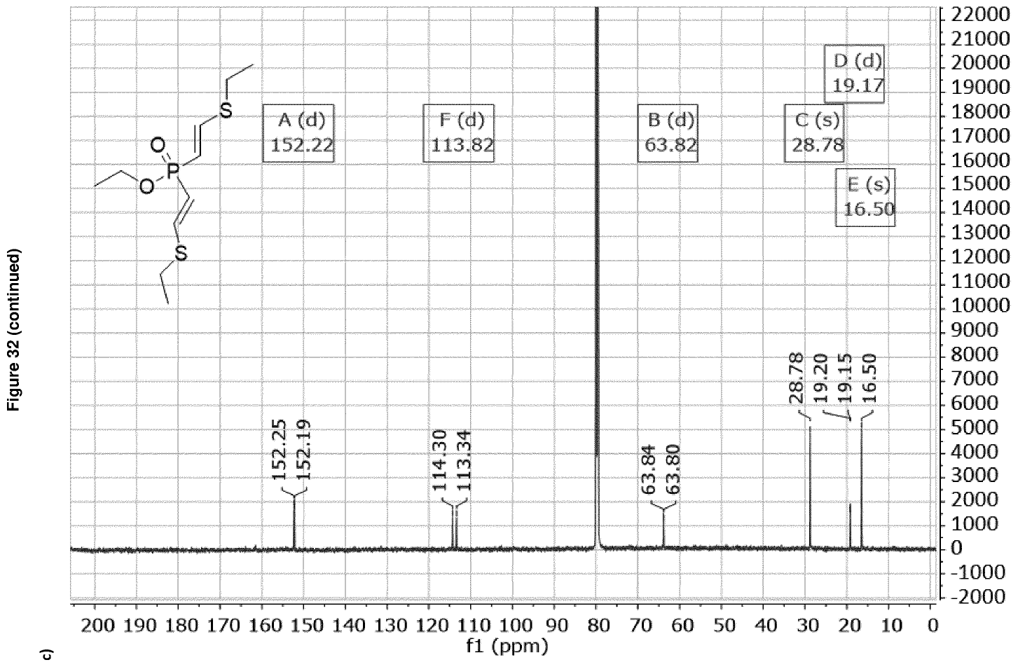

4 mg of the E/E-isomer were obtained as a dark orange liquid. NMR spectra of the E/E isomer are shown in FIG. 32 (a)¹H; b)³¹P; c)¹³C).

¹H NMR (600 MHz, CDCl₃) δ 7.50 (dd, J=20.0, 16.7 Hz, 2H), 5.68 (dd, J=19.2, 16.7 Hz, 2H), 4.11 (dd, J=7.9, 7.0 Hz, 2H), 2.90 (q, J=7.4 Hz, 4H), 1.41 (dt, J=14.2, 7.2 Hz, 9H).

¹³C NMR (151 MHz, CDCl₃) δ 152.22 (d, J=8.3 Hz), 113.82 (d, J=144.2 Hz), 63.82 (d, J=5.9 Hz), 28.78, 19.17 (d, J=6.7 Hz), 16.50.

³¹P-NMR (243 MHz, CDCl₃) δ 25.78.

HRMS for C₁₀H₂₀O₂PS2⁺ [M+H]⁺ calc.: 267.0637; found: 267.0645.

### General Procedure A

A 50 ml Schlenk flask was charged with 150 μl dimethylphosphoramidous dichloride (1 mmol, 1 eq.) under argon. 2 ml dry THF were added, and the mixture was cooled to −78° C. 4.4 ml ethynylmagnesium bromide (0.5 M in THF, 1.1 eq.) were added dropwise and the reaction was allowed to warm to room temperature. Then the mixture was again cooled to −78° C. and 1 eq. alcohol dissolved in 2.5 ml tetrazole-solution (0.45 M in acetonitrile, 1.1 eq.) and 2.5 ml acetonitrile were added dropwise. The reaction mixture was allowed to warm to room temperature. Subsequently the mixture was extracted from H₂O/DCM. Combined organic layers were dried and evaporated under reduced pressure. The crude was purified by column chromatography on silica gel (100% Et₂O) to afford the desired product as a phosphinite. The product fractions were combined and mixed with acetonitrile/H₂O (80:20) containing 0.1% TFA. Et₂O was removed under reduced pressure and H₂O₂ (0.5 ml of a 30% aqueous solution per mmol starting material) were added to the solution. Subsequently the reaction mixture was lyophilized to obtain the desired compound. The synthesis is also depicted in the following scheme, which further shows the synthesis of corresponding phosphonothioates and phosphinamidates:

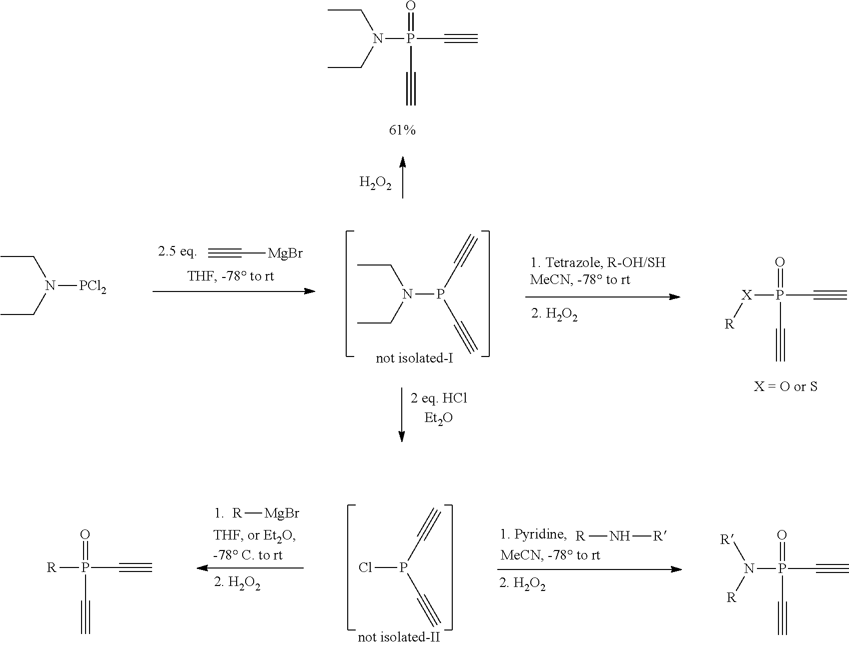

For the generation of the corresponding phosphinothioate the same procedure as for the phosphinates can be employed. Intermediate I is generated from diethylphosphoramidous dichloride and ethynylmagnesium bromide as described in the synthesis of diethynyl phosphinates. In brief, the mixture was again cooled to −78° C. and 1 eq. thiol dissolved in a 1:1 mixture tetrazole-solution (0.45 M in acetonitrile, 1.1 eq.) and acetonitrile were added dropwise. The reaction mixture was allowed to warm to room temperature. Subsequently the mixture was extracted from H₂O/DCM. Combined organic layers were dried and evaporated under reduced pressure. The crude was purified by column chromatography on silica gel (100% Et₂O) to afford the desired product as a phosphinite. The product fractions were combined and mixed with acetonitrile/H₂O (80:20) containing 0.1% TFA. Et₂O was removed under reduced pressure and H₂O₂ (0.5 ml of a 30% aqueous solution per mmol starting material) were added to the solution. Subsequently the reaction mixture was lyophilized to obtain the desired compound.

For the generation of the corresponding phosphinamidates Intermediate I would be treated with 2 eq. HCl in Et₂O at 0° C. to form intermediate II, as described by Van Assema et al. (S. G. A. van Assema, P. B. Kraikivskii, S. N. Zelinskii, V. V. Saraev, G. B. de Jong, F. J. J. de Kanter, M. Schakel, J. Chris Slootweg, K. Lammertsma, Building blocks for phospha[n]pericyclynes, J. Organomet. Chem. 692 (2007) 2314-2323. https://doi.org/10.1016/j.jorganchem.2007.02.017). After cooling to −78° C. the secondary amine dissolved in Et₂O or THF or MeCN with 1.2 eq. pyridine would be added dropwise to the virgorously stirred reaction mixture. The reaction was warmed to r.t. and stirring was continued for 30 minutes. The reaction mixture would be extracted with DCM and purified over silica. Finally, purified compounds would be dissolved in a mixture of water and MeCN, oxidized with hydrogenperoxide and lyophilized to obtain the desired product.

Alternatively, intermediate II can be reacted with a Grignard compound. Accordingly, after cooling to −78° C. the Grignard compound R—MgBr dissolved in THF or Et₂O would be added dropwise to the vigorously stirred reaction mixture. The reaction was warmed to r.t. and stirring was continued for 30 minutes. The reaction mixture would be extracted with DCM and purified over silica. Finally, purified compounds would be dissolved in a mixture of water and MeCN, oxidized with hydrogenperoxide and lyophilized to obtain the desired product.

### But-3-yn-1-yl diethynylphosphinate (2)

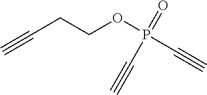

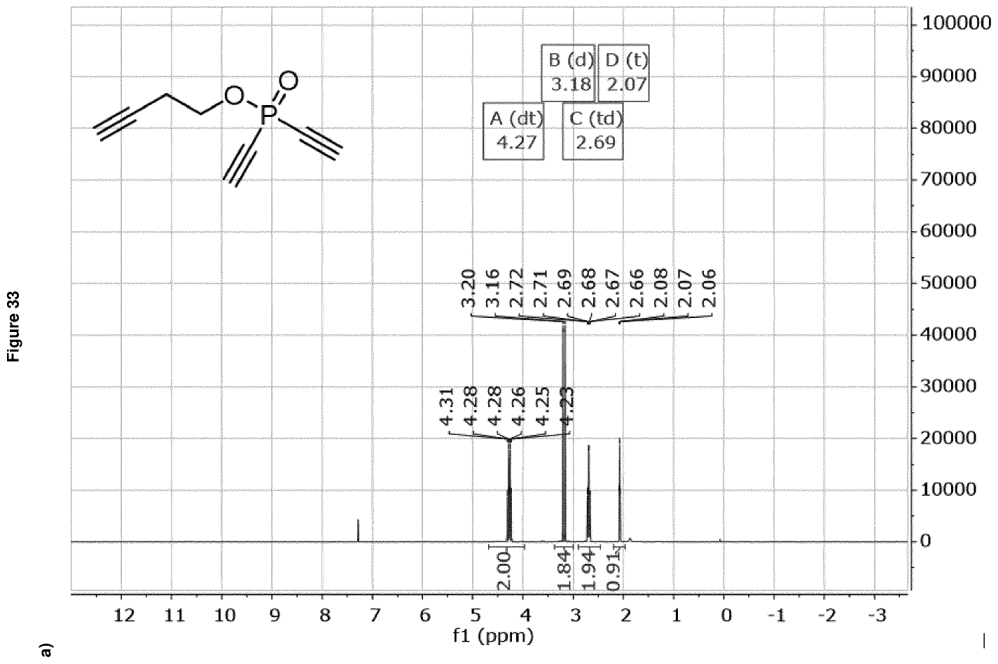

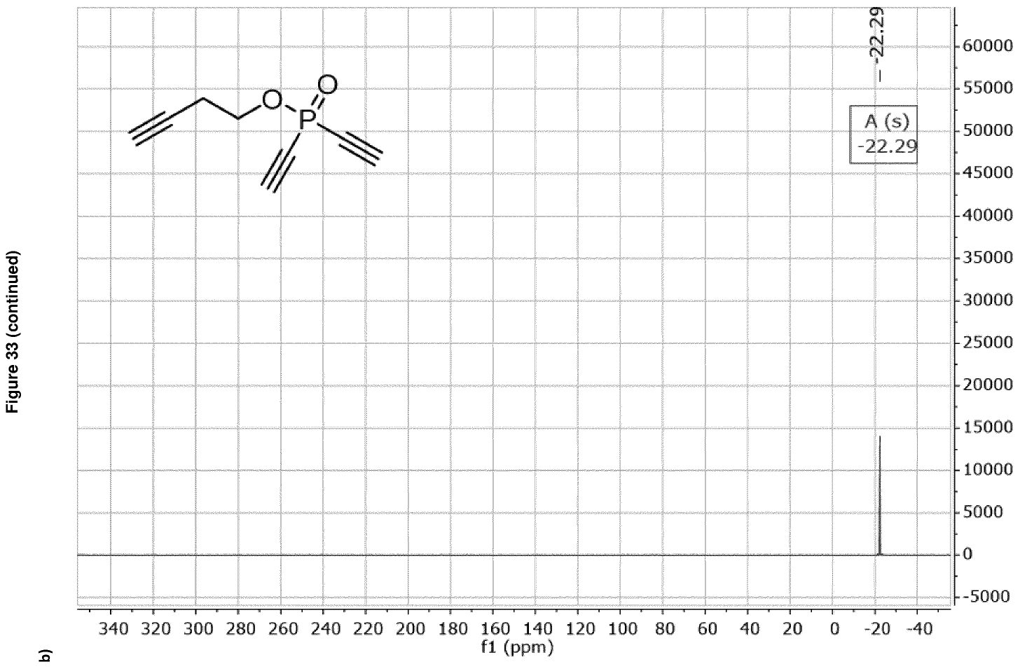

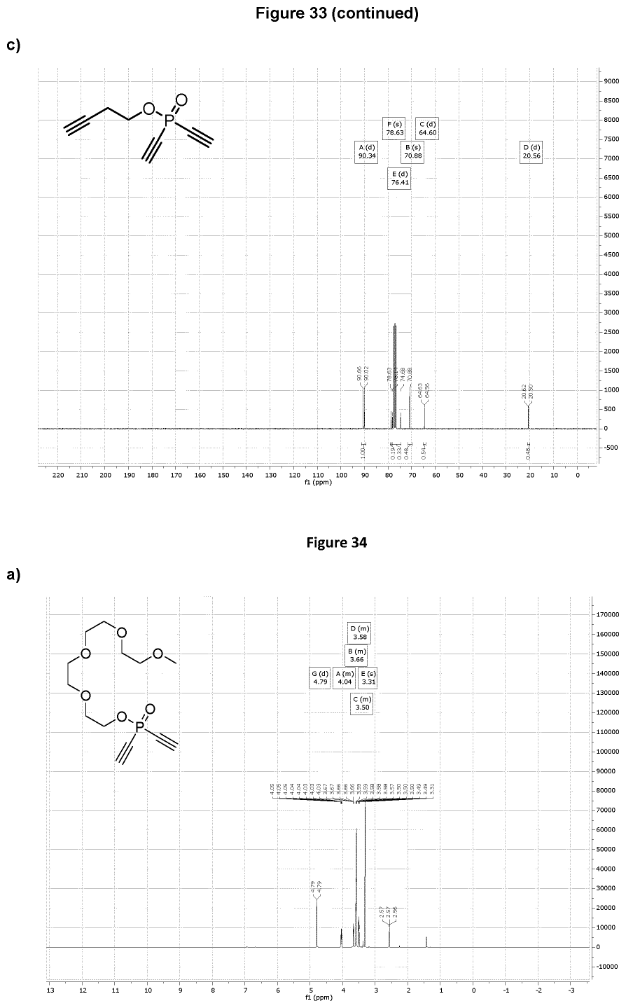

The compound was synthesized according to general procedure A and was obtained as a clear colorless liquid. (102 mg, 61% yield). NMR spectra of 2 are shown in FIG. 33 (a) ¹H; b)³¹P; C)¹³C).

¹H-NMR (300 MHz, CDCl₃) δ4.27 (dt, J=9.4, 7.1 Hz, 2H), 3.18 (d, J=12.8 Hz, 2H), 2.69 (td, J=7.1, 2.7 Hz, 2H), 2.07 (t, J=2.7 Hz, 1H).

¹C-NMR (75 MHz, CDCl₃) δ 90.34 (d, J=48.6 Hz), 78.63, 76.41 (d, J=260.9 Hz), 70.88, 64.60 (d, J=5.5 Hz), 20.56 (d, J=8.6 Hz).

³¹P-NMR (122 MHz, CDCl₃) δ−22.29.

HRMS for C₈H₈O₂P⁺ [M+H]⁺ calc.: 167.0256; found: 167.0258.

mPEG₄ diethynylphosphinate (3)

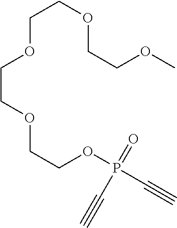 [SMILES: C#CP(=O)(C#C)OCCOCCOCCOCCOC]

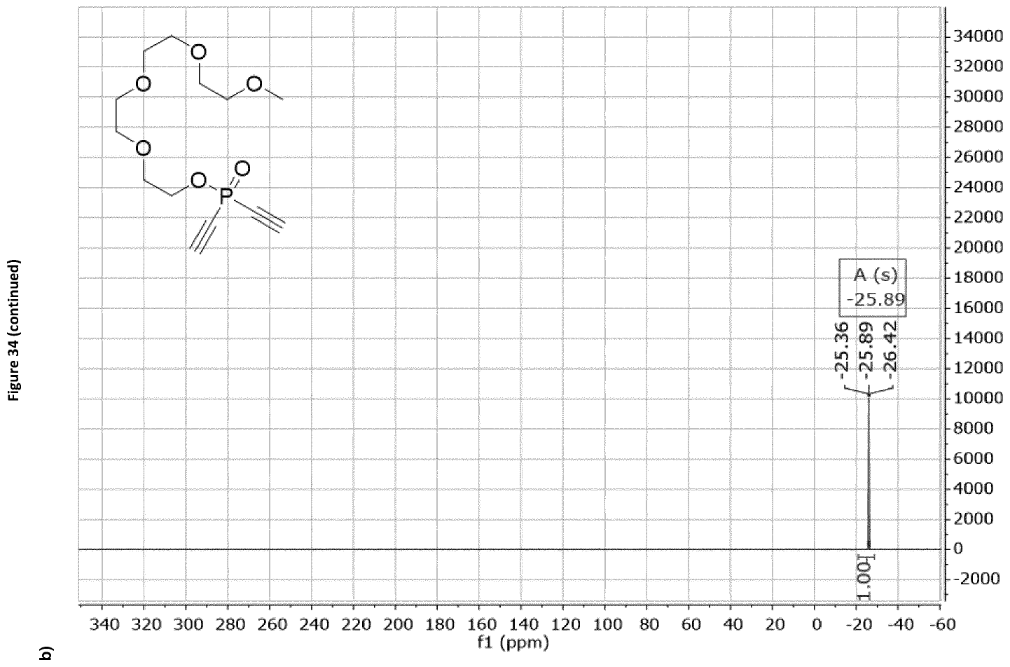

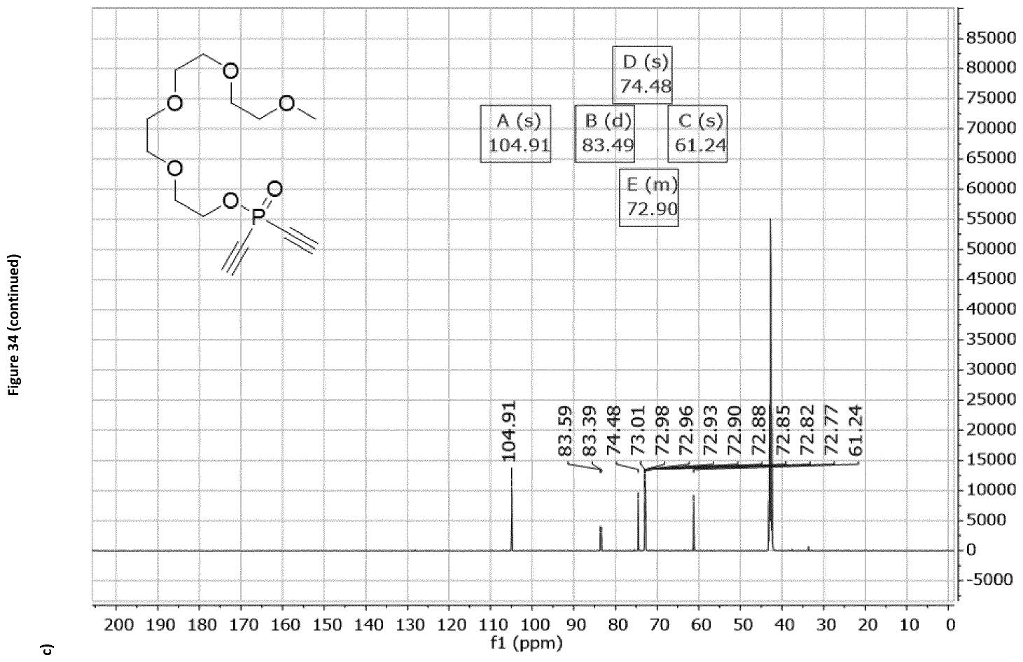

The compound was synthesized according to general procedure A and was obtained as a light brown liquid. (84 mg, 24% yield). NMR spectra of 3 are shown in FIG. 34 (a) ¹H; b) ³¹P; c) ¹³C).

¹H NMR (600 MHz, DMSO-d₆) δ 4.79 (d, J=1.5 Hz, 2H), 4.07-3.99 (m, 2H), 3.69-3.64 (m, 2H), 3.63-3.54 (m, 10H), 3.52-3.46 (m, 2H), 3.31 (s, 3H).

¹³C NMR (151 MHz, DMSO-d₆) δ104.91 (2C), 83.49 (d, J=30.3 Hz, 2C), 74.48 (2C), 73.17-72.57 (m, 6C), 61.24.

³¹P-NMR (122 MHz, CDCl₃) δ−25.89.

HRMS for C₁₃H₂₁O₆P⁺ [M+H]⁺ calc.: 305.1149; found: 305.1148.

NBD-methylamino-hexanol (A1)

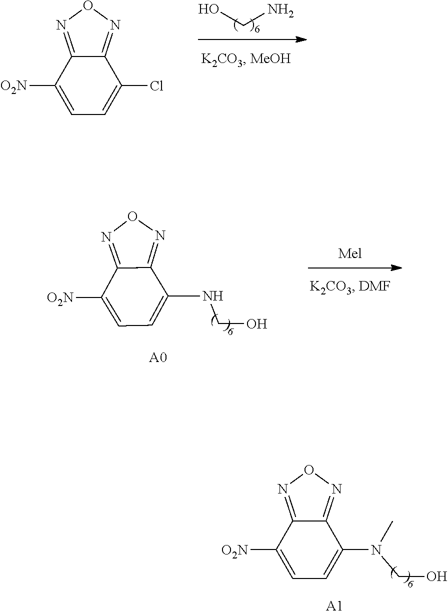

A1 was synthesized according to a two-step procedure reported by Concilio et al. (Concilio, S.; Ferrentino, I.; Sessa, L.; Massa, A.; lannelli, P.; Diana, R.; Panunzi, B.; Rella, A.; Piotto, S. A Novel Fluorescent Solvatochromic Probe for Lipid Bilayers. Supramol. Chem. 2017, 29 (11), 887-895. https://doi.org/10.1080/10610278.2017.1372583) and was obtained as a dark-red solid in 37% yield. Spectroscopic characterization was in good agreement with the literature.

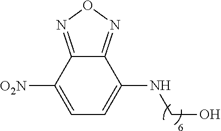

A0: ¹H NMR (600 MHz, Chloroform-d) δ 8.57 (d, J=8.6 Hz, 1H), 7.74 (d, J=7.8 Hz, 1H), 6.25 (d, J=8.6 Hz, 1H), 3.78 (t, J=6.3 Hz, 2H), 3.65-3.49 (m, 2H), 1.93 (m, 2H), 1.77-1.53 (m, 6H).

¹³C NMR (151 MHz, Chloroform-d) δ 146.63, 139.19, 133.15, 131.23, 126.73, 101.21, 65.43, 46.61, 34.90, 31.04, 29.35, 28.18.

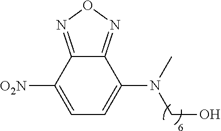

A1: ¹H NMR (600 MHz, Methanol-d₄) δ 8.46 (d, J=9.1 Hz, 1H), 6.32 (d, J=9.2 Hz, 1H), 4.11 (broad-s, 2H), 3.58 (t, J=6.5 Hz, 2H), 3.01 (s, 3H), 1.93-1.75 (m, 2H), 1.68-1.52 (m, 2H), 1.53-1.42 (m, 4H). ¹³C NMR (151 MHz, Methanol-d₄) δ 163.44, 145.94, 144.85, 135.67, 120.74, 101.39, 61.38, 55.42, 35.55, 32.06, 30.26, 26.12, 25.26.

HRMS for C₁₃H₁₈N₄O₄ [M+H]⁺ calc.: 295.1401; found: 295.1402

### NBD Diethynyl-Phosphinate (4)

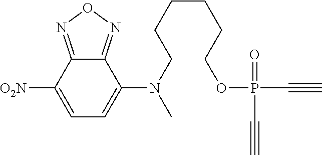 [SMILES: C#CP(=O)(C#C)OCCCCCCN(C)c1ccc([N+](=O)[O-])c2nonc12]

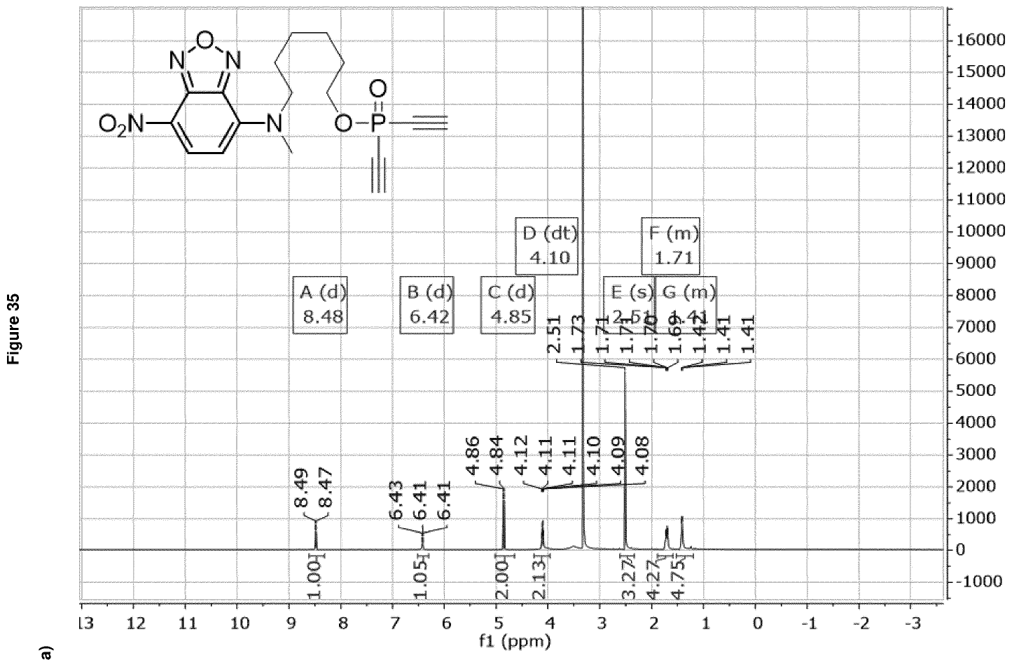

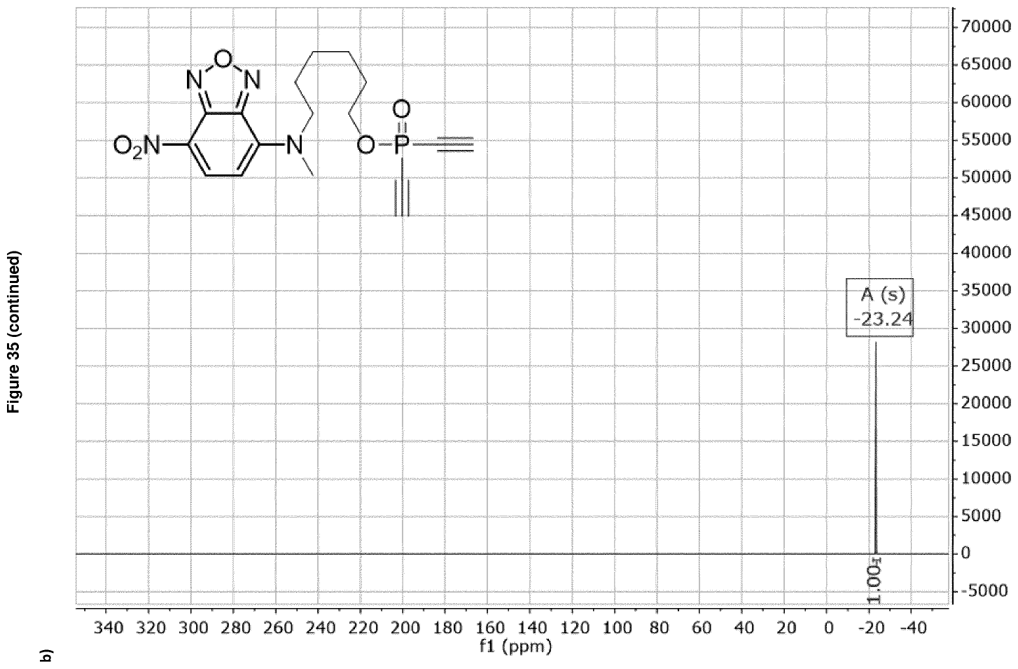

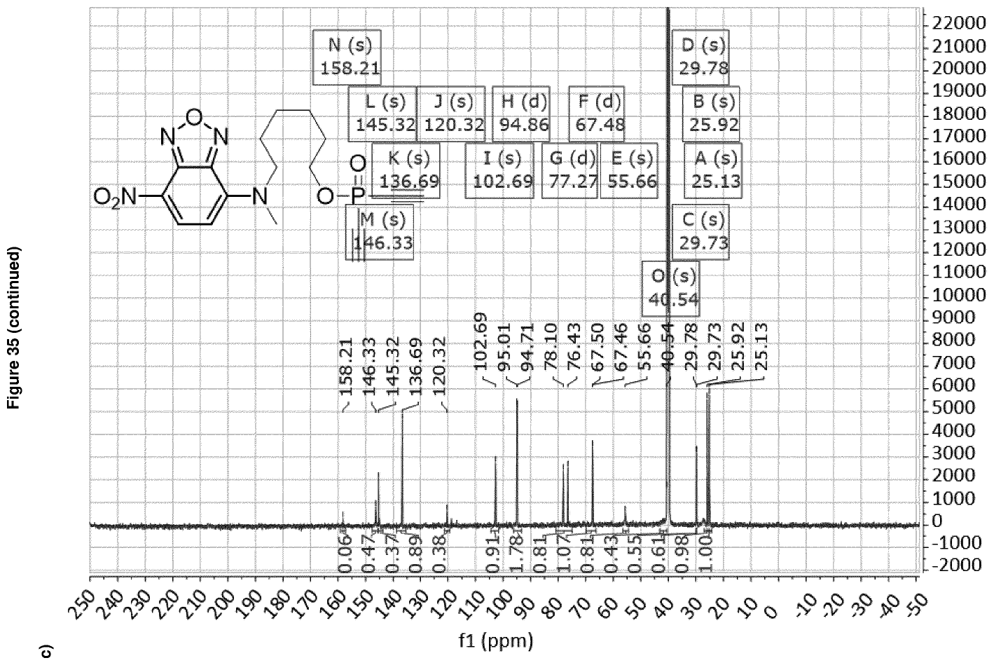

The compound was synthesized according to general procedure A (0.1 mmol-scale) and was obtained as a red-solid. (18 mg, 46% yield). NMR spectra of 4 are shown in FIG. 35 (a) ¹H; b)³¹P; c)¹³C).

¹H NMR (600 MHz, DMSO-d₆) δ 8.48 (d, J=9.2 Hz, 1H), 6.42 (d, J=9.1 Hz, 1H), 4.85 (d, J=12.7 Hz, 2H), 4.10 (dt, J=9.3, 6.3 Hz, 2H), 2.51 (s, 3H), 1.88-1.59 (m, 4H), 1.53-1.19 (m, 5H).

¹³C NMR (151 MHz, DMSO-d₆) δ 158.21, 146.33, 145.32, 136.69, 120.32, 102.69, 94.86 (d, J=45.3 Hz), 77.27 (d, J=251.5 Hz), 67.48 (d, J=6.2 Hz), 55.66, 40.54, 29.78, 29.73, 25.92, 25.13.

³¹P NMR (243 MHz, DMSO-d₆) δ-23.24.

HRMS for C₁₇H₂₀N₄O₅P⁺ [M+H]⁺ calc.: 391.1166; found: 391.1198.

Diethyl diethynylphosphinic amide (II)

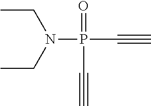

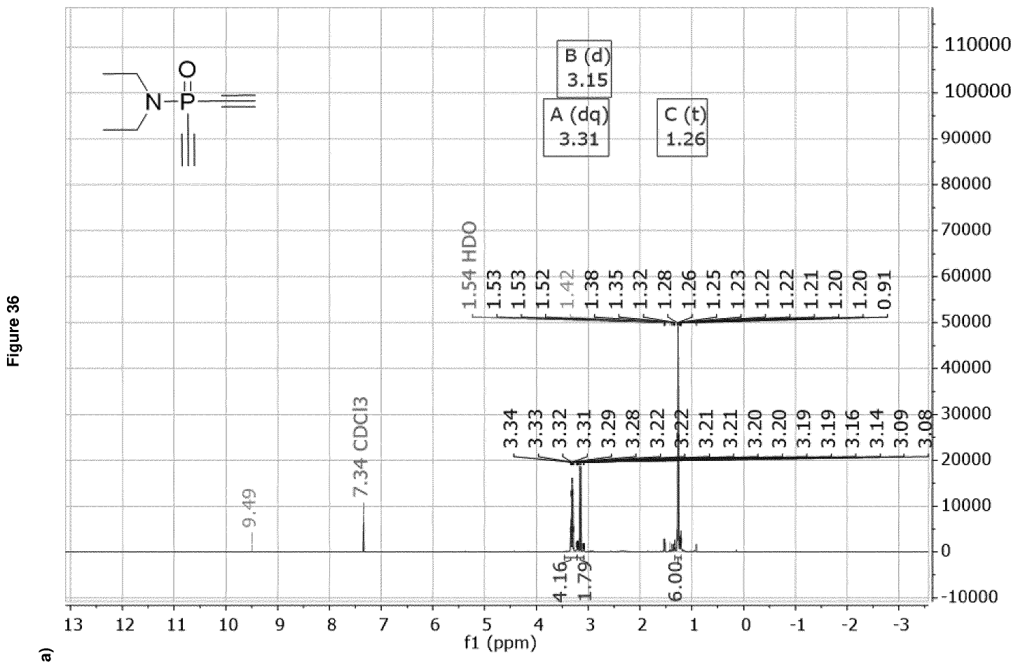

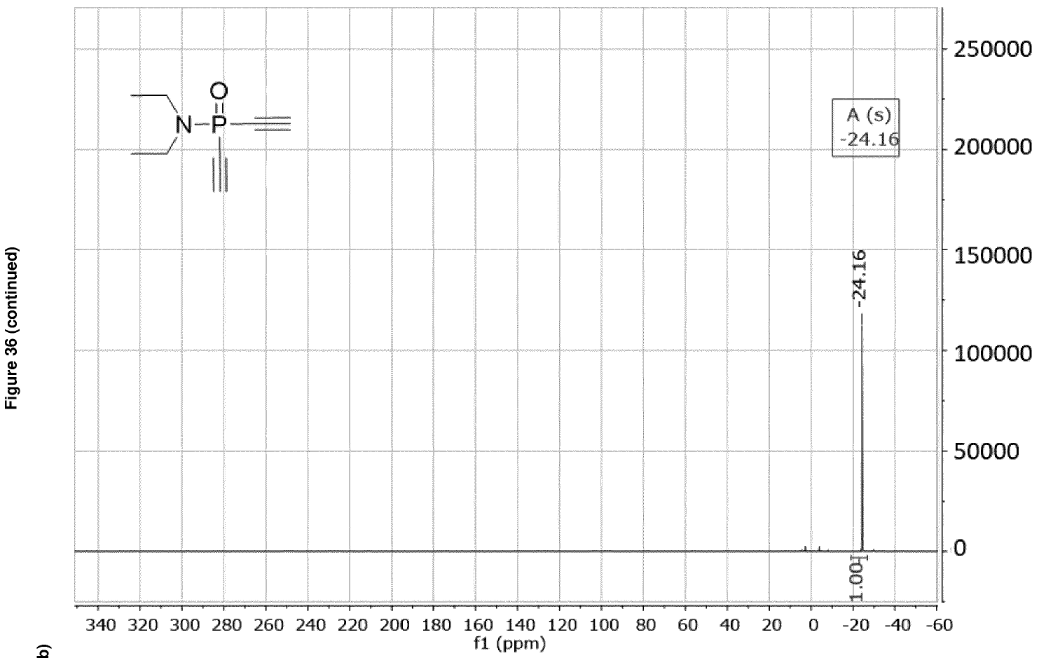

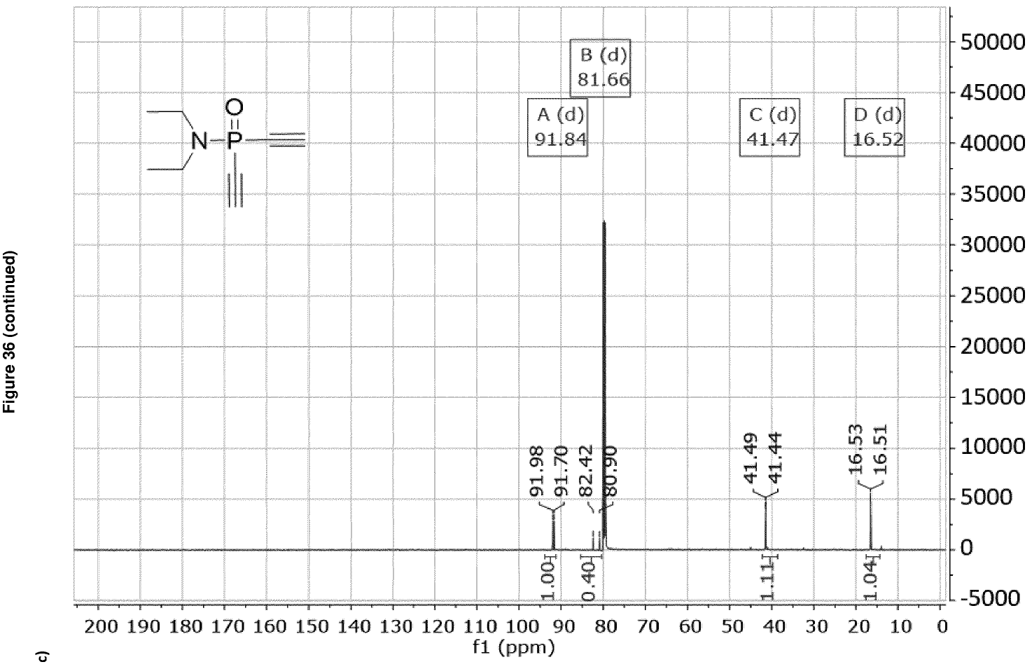

A 50 ml Schlenk-flask was charged with 300 μl diethyl-phosphoramidous (2 mmol, 1 eq.) dissolved in 4 ml of dry THF under argon. After cooling to −78° C., 2.5 eq. ethynyl-magnesiumbromide (0.5 M in THF, 10 ml) were added dropwise. After the reaction was allowed to warm to room temperature, the mixture was poured onto 150 ml ice-cold H₂O containing 10 mmol H₂O₂. The solution was extracted 3× with 30 ml DCM, combined organic layers were washed twice with 50 ml H₂O and dried over MgSO₄. After solvent evaporation, II was obtained as a brown solid. (205 mg, 61% yield). NMR spectra of II are shown in FIG. 36 (a) ¹H; b)³¹P; c)¹³C).

¹H-NMR (600 MHz, CDCl₃) δ=3.23 (dq, J=14.1, 7.1 Hz, 4H), 3.07 (d, J=11.6 Hz, 2H), 1.18 (t, J=7.1 Hz, 6H).

¹³C-NMR (151 MHz, CDCl₃) δ=89.47 (d, J=42.0 Hz), 79.29 (d, J=229.2 Hz), 39.09 (d, J=6.3 Hz), 14.15 (d, J=3.0 Hz).

³¹P-NMR (243 MHz, CDCl₃) δ=−24.16.

No HRMS could be recorded, due to P—N bond hydrolysis during analysis.

### EDANS-Azidopropionic Amide (EDANS-N₃)

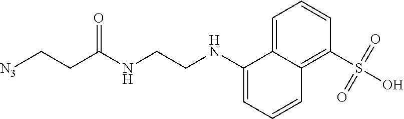 [SMILES: [N-]=[N+]=NCCC(=O)NCCNc1cccc2c(S(=O)(=O)O)cccc12]

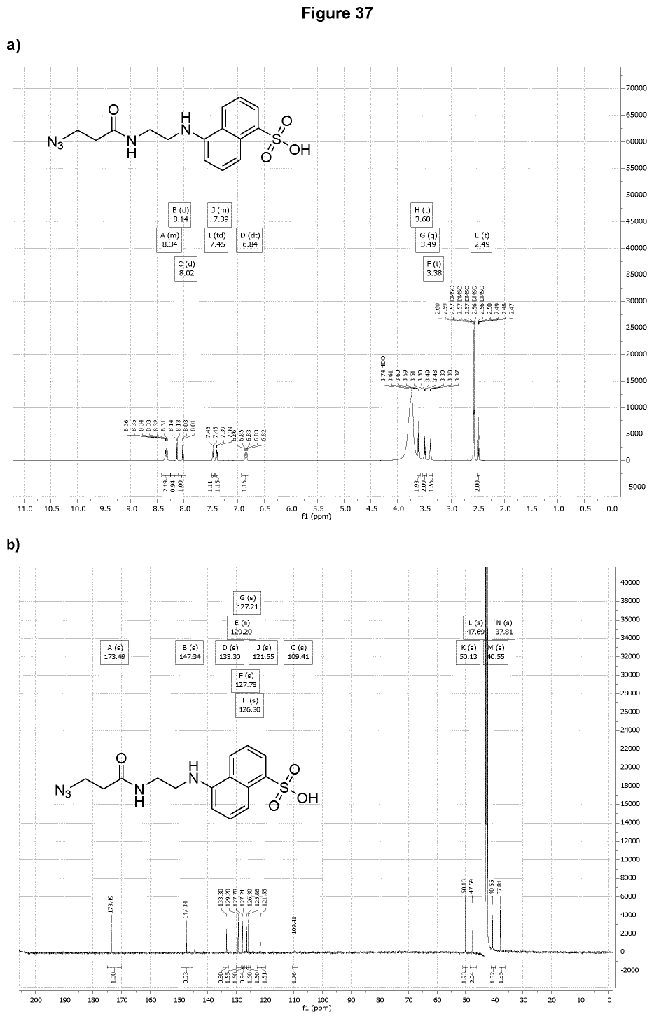

100 mg azido-propionic NHS-ester and 115 mg EDANS sodium salt were dissolved in 10 ml DCM/DMF (8:2) and 200 μl DIPEA were added. After the reaction completed, the solvent was evaporated and the crude was purified via semi-preparative HPLC. (grey solid, 76 mg, 50%). NMR spectra of EDANS-N₃ are shown in FIG. 37 (a) ¹H; b)¹³C).

¹H NMR (600 MHz, DMSO-d₆) δ 8.42-8.26 (m, 2H), 8.14 (d, J=8.5 Hz, 1H), 8.02 (d, J=7.1 Hz, 1H), 7.45 (td, J=7.8, 2.7 Hz, 1H), 7.42-7.36 (m, 1H), 6.84 (dt, J=11.2, 7.1 Hz, 1H), 3.60 (t, J=6.4 Hz, 2H), 3.49 (q, J=6.3 Hz, 2H), 3.38 (t, J=6.4 Hz, 2H), 2.49 (t, J=6.4 Hz, 2H).

¹³C NMR (151 MHz, DMSO-d₆) δ 173.49, 147.34, 133.30, 129.20, 127.78, 127.21, 126.30, 125.86, 121.55, 109.41, 50.13, 47.69, 40.55, 37.81.

### General Procedure for Peptide Synthesis

Peptides were synthesized in a 0.05 mmol scale on a Rink amide resin with a loading of 0.78 mmol/g. The synthesis was carried out on a PTI synthesizer with single couplings of each amino acid (5 eq. amino acid, 5 eq. HCTU, 5 eq. Oxyma, 10 eq. DIPEA for 40 min) in DMF. The last amino acid was coupled with a Boc-protected N-terminus. Finally the peptides were cleaved from the resin by treatment with 2 ml of a TFA/TIS/H₂O (95:2.5:2.5) mixture for 1 h and precipitated in cold Et₂O. The crude peptides were purified by semi-preparative HPLC.

### Peptide 1

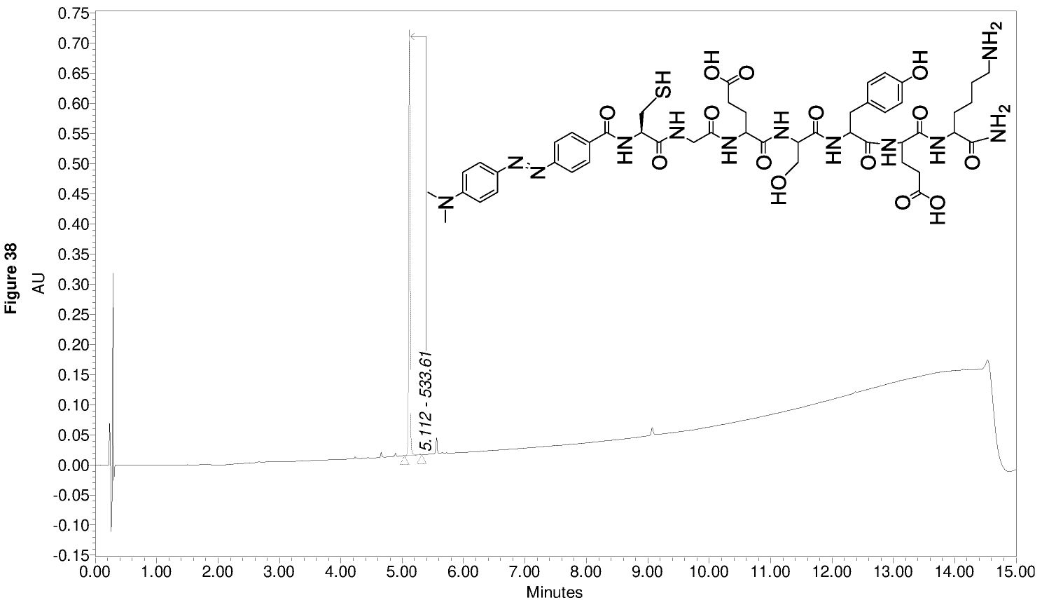

Peptide 1 was synthesized according to the general procedure for peptide synthesis. DABCYL-COOH was coupled as final step using the following conditions: 3 eq. acid, 3 eq. HATU, 10 eq. DIPEA for 60 minutes in DMF. The Peptide was purified via semi preparative HPLC and obtained as a dark-red powder. (39.1 mg, 18 μmol, 36%). An HPLC chromatogram of Peptide 1 is shown in FIG. 38.

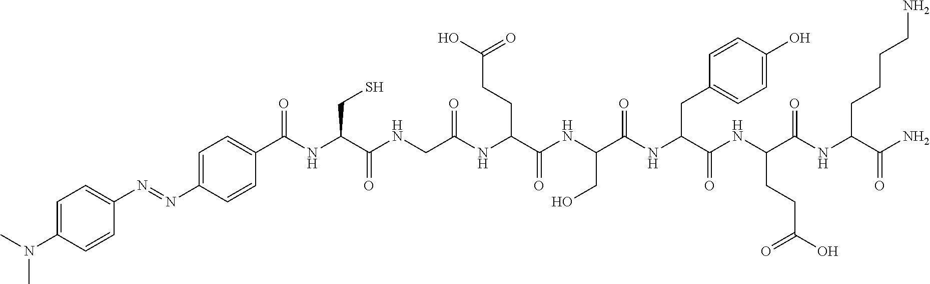 [SMILES: CN(C)c1ccc(/N=N/c2ccc(C(=O)N[C@@H](CS)C(=O)NCC(=O)NC(CCC(=O)O)C(=O)NC(CO)C(=O)NC(Cc3ccc(O)cc3)C(=O)NC(CCC(=O)O)C(=O)NC(CCCCN)C(N)=O)cc2)cc1]

ESI-MS for C₄₈H₆₆N₁₂O₁₄S: [M+2H]²⁺ calc.: 533.23; found: 533.34

### FRET-Pair 1 (F1)

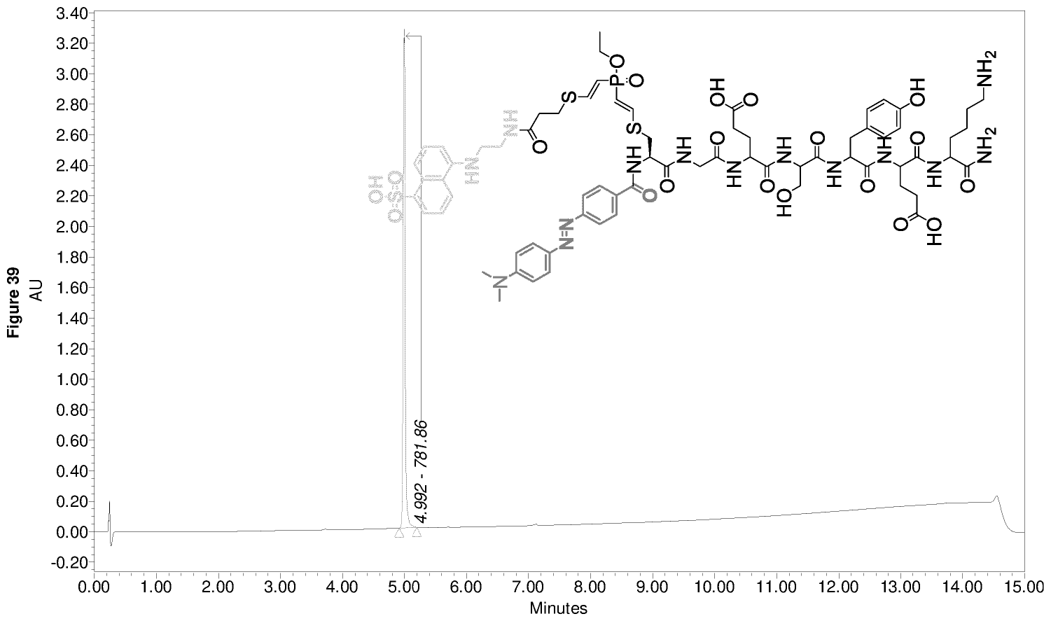

Quenched FRET-Pair 1 was synthesized from peptide 2 and excess (10 eq.) phosphinate 1 in PBS (pH 7.4). After purification of the intermediate, it was reacted with 1.2 eq. EDANS-thiol in PBS. F1 was purified via semi-preparative HPLC (1.67 mg, 86%). An HPLC chromatogram of F1 is shown in FIG. 39.

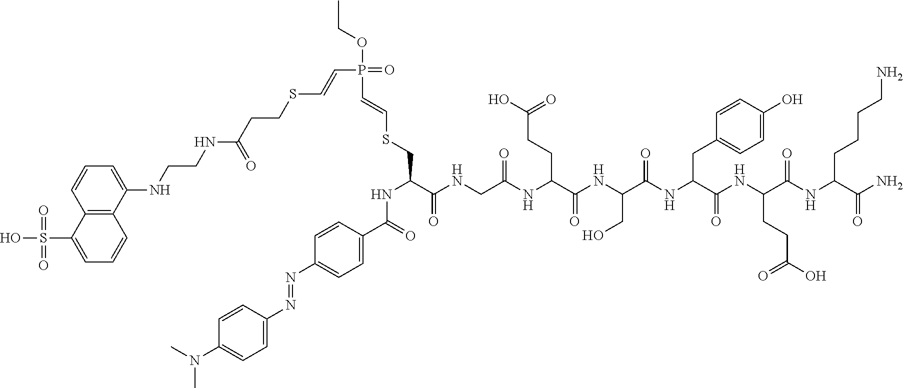 [SMILES: CCOP(=O)(/C=C/SCCC(=O)NCCNc1cccc2c(S(=O)(=O)O)cccc12)/C=C/SC[C@H](NC(=O)c1ccc(/N=N/c2ccc(N(C)C)cc2)cc1)C(=O)NCC(=O)NC(CCC(=O)O)C(=O)NC(CO)C(=O)NC(Cc1ccc(O)cc1)C(=O)NC(CCC(=O)O)C(=O)NC(CCCCN)C(N)=O]

HRMS for C₆₉H₈₉N₁₄O₂₀PS₃: [M+3H]³⁺ calc.: 521.1832; found: 521.1806
[M+2H]²⁺ calc.: 781.2711; found: 781.2713

### FRET-Pair 2 (F2)

Quenched FRET-Pair 2 was synthesized analogously to F1, only phosphinate 2 was used as a linker. F2 was purified via semi-preparative HPLC (1.94 mg, 91%). An HPLC chromatogram of F2 is shown in FIG. 40.

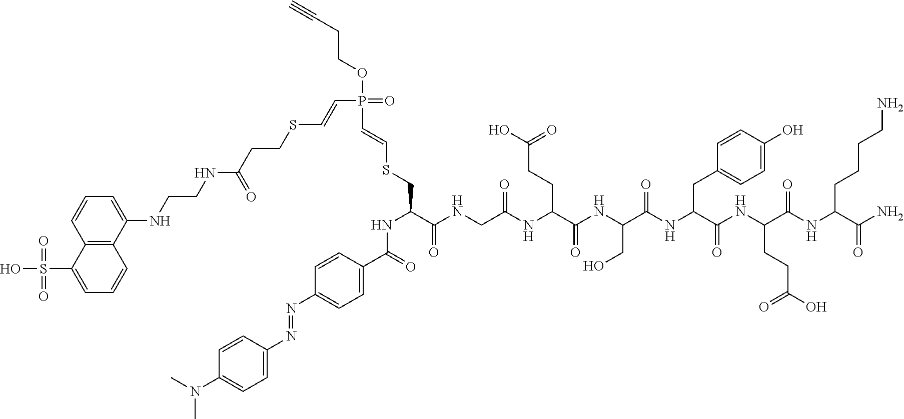 [SMILES: C#CCCOP(=O)(/C=C/SCCC(=O)NCCNc1cccc2c(S(=O)(=O)O)cccc12)/C=C/SC[C@H](NC(=O)c1ccc(/N=N/c2ccc(N(C)C)cc2)cc1)C(=O)NCC(=O)NC(CCC(=O)O)C(=O)NC(CO)C(=O)NC(Cc1ccc(O)cc1)C(=O)NC(CCC(=O)O)C(=O)NC(CCCCN)C(N)=O]

HRMS for C₇₁H₈₉N₁₄O₂₀PS₃: [M+3H]³⁺ calc.: 529.1832; found: 521.1826
[M+2H]²⁺ calc.: 793.2711; found: 793.2737

### FRET-Pair 3 (F3)

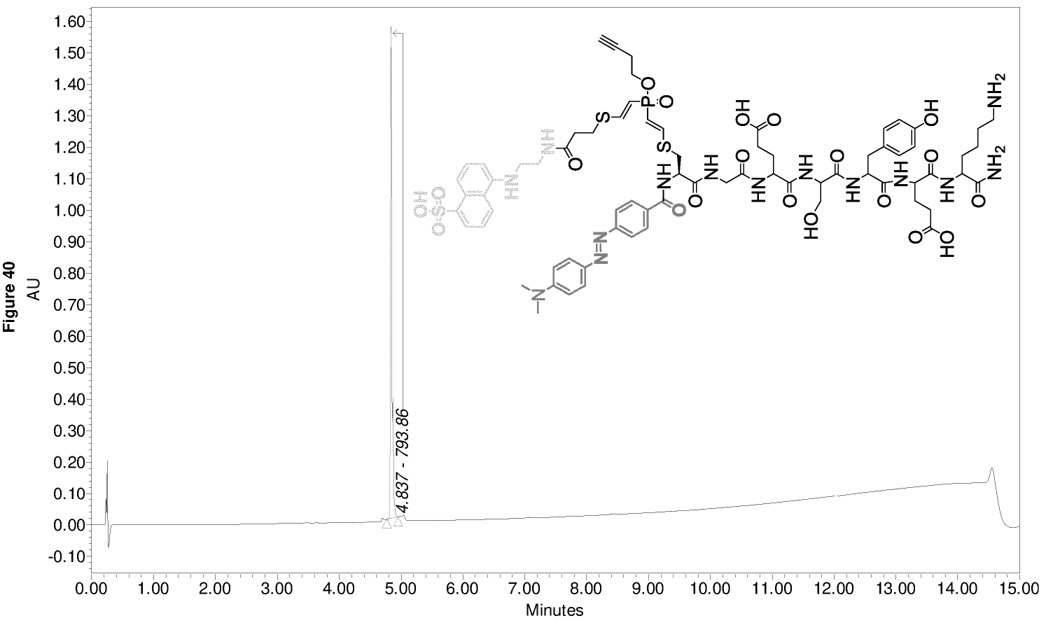

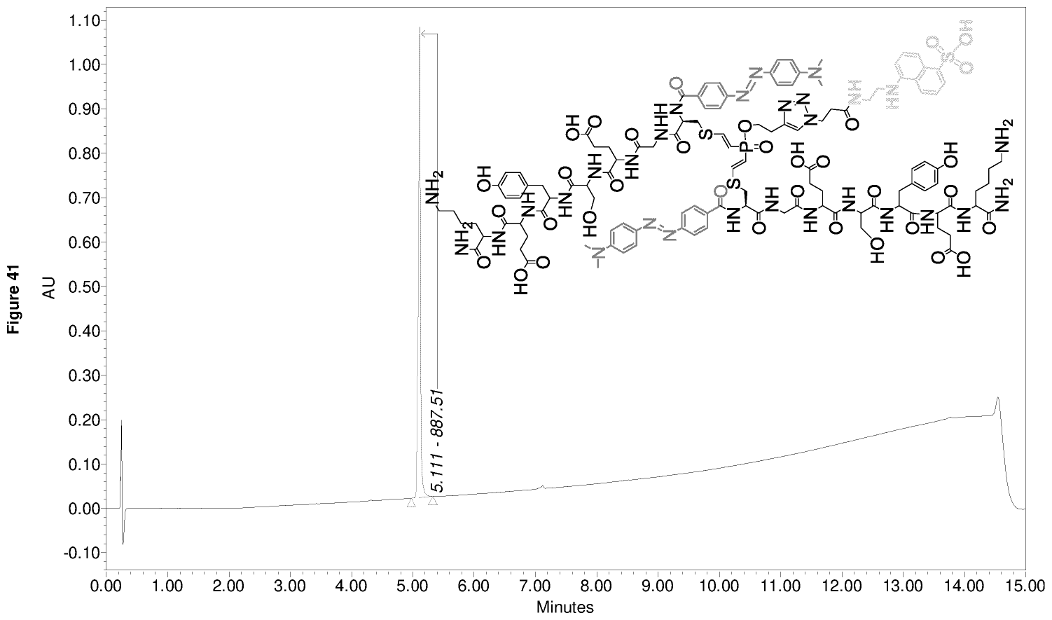

Quenched FRET-Pair 3 was synthesized from 2 eq. peptide 2 and phosphinate 2 (1 eq.) in PBS. Subsequently, EDANS-N₃ was conjugated to the phosphinate side-chain using CuBr (10 mol %) as catalyst. F3 was purified via semi-preparative HPLC (2.13 mg, 78%). An HPLC chromatogram of F3 is shown in FIG. 41.

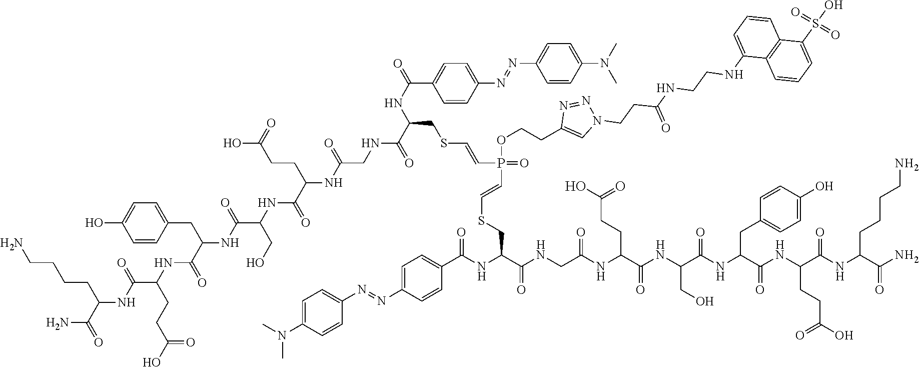 [SMILES: CN(C)c1ccc(/N=N/c2ccc(C(=O)N[C@@H](CS/C=C/P(=O)(/C=C/SC[C@H](NC(=O)c3ccc(/N=N/c4ccc(N(C)C)cc4)cc3)C(=O)NCC(=O)NC(CCC(=O)O)C(=O)NC(CO)C(=O)NC(Cc3ccc(O)cc3)C(=O)NC(CCC(=O)O)C(=O)NC(CCCCN)C(N)=O)OCCc3cn(CCC(=O)NCCNc4cccc5c(S(=O)(=O)O)cccc45)nn3)C(=O)NCC(=O)NC(CCC(=O)O)C(=O)NC(CO)C(=O)NC(Cc3ccc(O)cc3)C(=O)NC(CCC(=O)O)C(=O)NC(CCCCN)C(N)=O)cc2)cc1]

HRMS for C₁₁₉H₁₅₂N₂₉O₃₄PS₃: [M+3H]³⁺ calc.: 887.0058; found: 887.0068
[M+4H]⁴⁺ calc.: 665.5062; found: 665.5032

### FRET-Pair 4 (F4)

Quenched FRET-Pair 4 was synthesized from peptide 2 and excess (10 eq.) phosphinate 1 in PBS (pH 7.4). After purification of the intermediate, it was reacted with 1.2 eq. EDANS-N₃ in PBS and 20 mol % CuBr. F4 was purified via semi-preparative HPLC (2.73 mg, 89%).

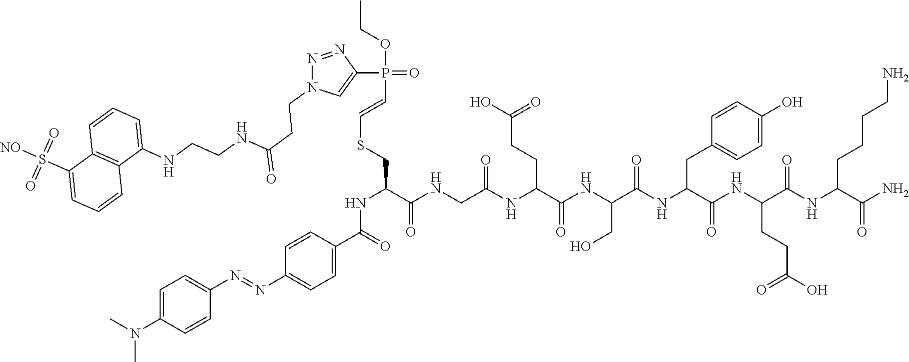

HRMS for C₆₉H₈₈N₁₇O₂₀PS₂: [M+3H]³⁺ calc.: 524.1930; found: 524.1885
[M+2H]²⁺ calc.: 785.7858; found: 785.7858

### EDANS-SH

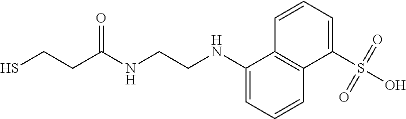 [SMILES: O=C(CCS)NCCNc1cccc2c(S(=O)(=O)O)cccc12]

50 mg tritylthio-propionic acid, 60 mg HATU and 200 μl DIPEA were dissolved in 5 ml DMF and stirred for 5 minutes. 40 mg EDANS sodium salt and were added and the reaction was allowed to proceed over night. The solvent was removed under reduced pressure and the residue was dissolved in TFA/DCM (1:1). After 30 minutes, solvent was removed under a stream of argon and the crude was purified via semi-preparative HPLC. (white powder, 21.3 mg, 45%)

¹H NMR (600 MHz, DMSO-d₆) δ 8.29 (d, J=8.6 Hz, 1H), 8.17 (t, J=5.8 Hz, 1H), 8.07 (dt, J=8.4, 1.1 Hz, 1H), 7.96 (dd, J=7.1, 1.1 Hz, 1H), 7.39 (dd, J=8.5, 7.1 Hz, 1H), 7.33 (dd, J=8.6, 7.5 Hz, 1H), 6.77 (d, J=7.5 Hz, 1H), 3.43 (q, J=6.3 Hz, 2H), 3.32 (t, J=6.5 Hz, 2H), 2.69 (q, J=7.1 Hz, 2H), 2.43 (t, J=7.0 Hz, 2H), 2.31 (t, J=8.0 Hz, 1H).

¹³C NMR (151 MHz, DMSO-d₆) δ 171.51, 144.71, 130.60, 126.46, 125.02, 124.45, 123.55, 123.09, 118.79, 45.10, 40.53, 37.79, 20.37.

HRMS for C₁₅H₁₉N₂O₄S₂⁺ [M+H]⁺ calc.: 355.0781 found: 355.0789.

EDANS-ethyl-thiovinyl-ethynylphosphinate (CS265)

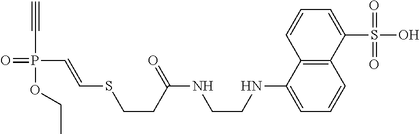 [SMILES: C#CP(=O)(/C=C/SCCC(=O)NCCNc1cccc2c(S(=O)(=O)O)cccc12)OCC]

CS265 was synthesized from ethyl-diethynyl-phosphinate (1, 5 eq.) and EDANS-SH (6.5 mg 1 eq.) dissolved in 400 μl DMSO/PBS (1:1) at room temperature. The reaction was stirred for 90 minutes and stopped by the addition of 3 ml 0.1% aq. TFA. CS265 was purified via semi-preparative HPLC. (6.9 mg, 76% yield).

¹H NMR (600 MHz, DMSO-d₆) δ 8.30-8.23 (m, 2H), 8.13 (d, J=8.5 Hz, 1H), 8.00 (dd, J=7.1, 1.1 Hz, 1H), 7.65 (dd, J=46.6, 12.5 Hz, 1H), 7.42 (dd, J=8.5, 7.1 Hz, 1H), 7.35 (dd, J=8.6, 7.6 Hz, 1H), 6.71 (d, J=7.6 Hz, 1H), 5.73 (dd, J=19.3, 12.5 Hz, 1H), 4.52 (d, J=10.8 Hz, 1H), 4.07 (dq, J=9.0, 7.0 Hz, 2H), 3.47 (q, J=6.3 Hz, 2H), 3.35 (t, J=6.6 Hz, 2H), 3.11 (t, J=7.1 Hz, 2H), 1.32 (t, J=7.0 Hz, 3H).

¹³C NMR (189 MHz, DMSO-d₆) δ 170.97, 154.21, 144.67, 143.14, 140.39, 130.61, 126.54, 124.88, 124.21, 123.15, 117.35, 112.44 (d, J=163.3 Hz), 104.57, 92.98 (d, J=36.1 Hz), 78.50 (d, J=201.7 Hz), 61.78 (d, J=5.9 Hz), 44.28, 38.09, 36.80, 31.03, 16.57 (d, J=6.8 Hz).

³¹P NMR (243 MHz, DMSO-d₆) δ 0.46.

EDANS-1,2,3-triazol-ethyl-ethynylphosphinate (CS266)

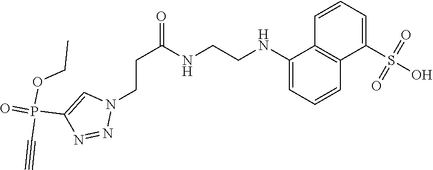 [SMILES: C#CP(=O)(OCC)c1cn(CCC(=O)NCCNc2cccc3c(S(=O)(=O)O)cccc23)nn1]

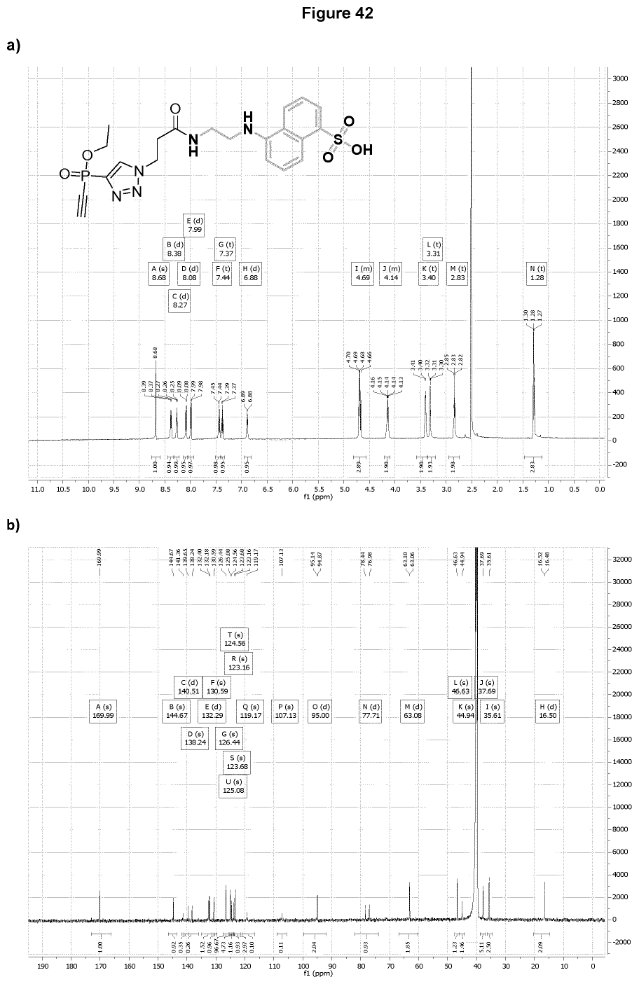

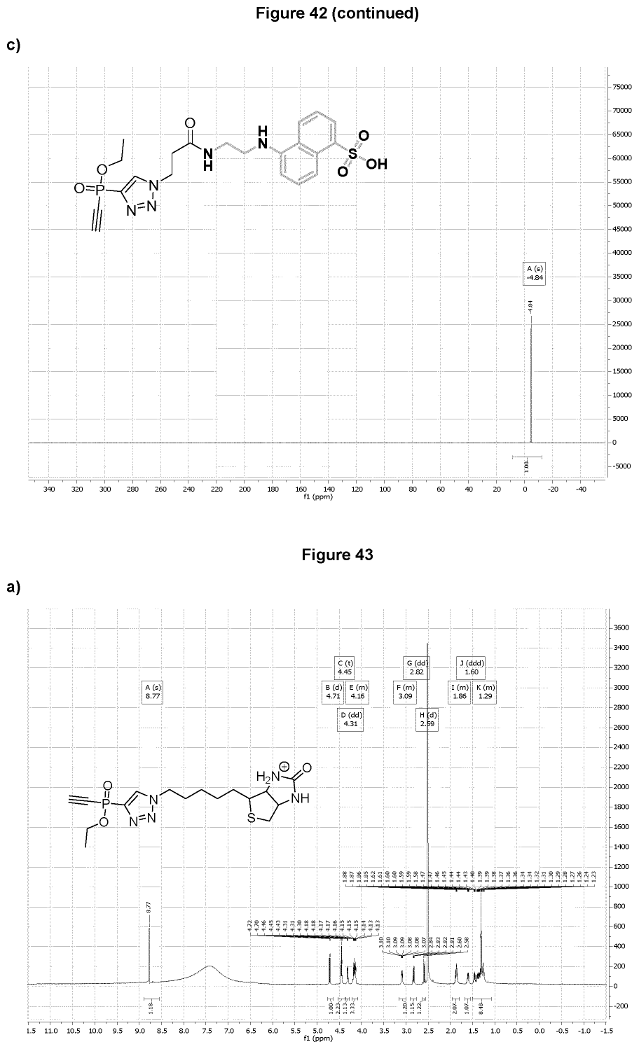

CS266 was synthesized from ethyl-diethynyl-phosphinate (1, 5 eq.) and EDANS-N₃ (8.3 mg 1 eq.) dissolved in 400 μl DMSO/PBS (1:1) at room temperature. The reaction was started by the addition of 5 mg CuBr. After 30 minutes, the reaction was stopped by the addition of 3 ml 0.1% aq. TFA and CS266 was purified via semi-preparative HPLC. (9.8 mg, 85% yield). NMR spectra of CS266 are shown in FIG. 42 (a)¹H; b)¹³C; c)³¹P).

¹H NMR (600 MHz, DMSO-d₆) δ 8.68 (s, 1H), 8.38 (d, J=8.7 Hz, 1H), 8.27 (d, J=5.7 Hz, 1H), 8.08 (d, J=8.5 Hz, 1H), 7.99 (d, J=7.1 Hz, 1H), 7.44 (t, J=7.9 Hz, 1H), 7.37 (t, J=8.1 Hz, 1H), 6.88 (d, J=7.5 Hz, 1H), 4.81-4.57 (m, 3H), 4.20-4.10 (m, 2H), 3.40 (t, J=6.2 Hz, 2H), 3.31 (t, J=6.6 Hz, 2H), 2.83 (t, J=6.8 Hz, 2H), 1.28 (t, J=7.0 Hz, 3H).

¹³C NMR (151 MHz, DMSO-d₆) δ 169.99, 144.67, 140.51 (d, J=257.5 Hz), 138.24, 132.29 (d, J=34.1 Hz), 130.59, 126.44, 125.08, 124.56, 123.68, 123.16, 119.17, 107.13, 95.00 (d, J=39.6 Hz), 77.71 (d, J=220.3 Hz), 63.08 (d, J=5.8 Hz), 46.63, 44.94, 37.69, 35.61, 16.50 (d, J=6.7 Hz).

³¹P NMR (243 MHz, DMSO-d₆) δ-4.84.

HRMS C₂₁H₂₅N₅O₆PS₂⁺ [M+H⁺] calc.: 506.1258; found:

Biotin-1,2,3-triazol-ethyl-ethynylphosphinate (CS292)

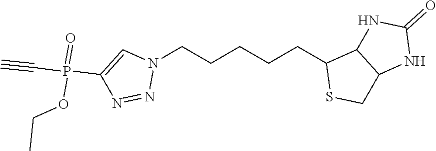 [SMILES: C#CP(=O)(OCC)c1cn(CCCCCC2SCC3NC(=O)NC32)nn1]

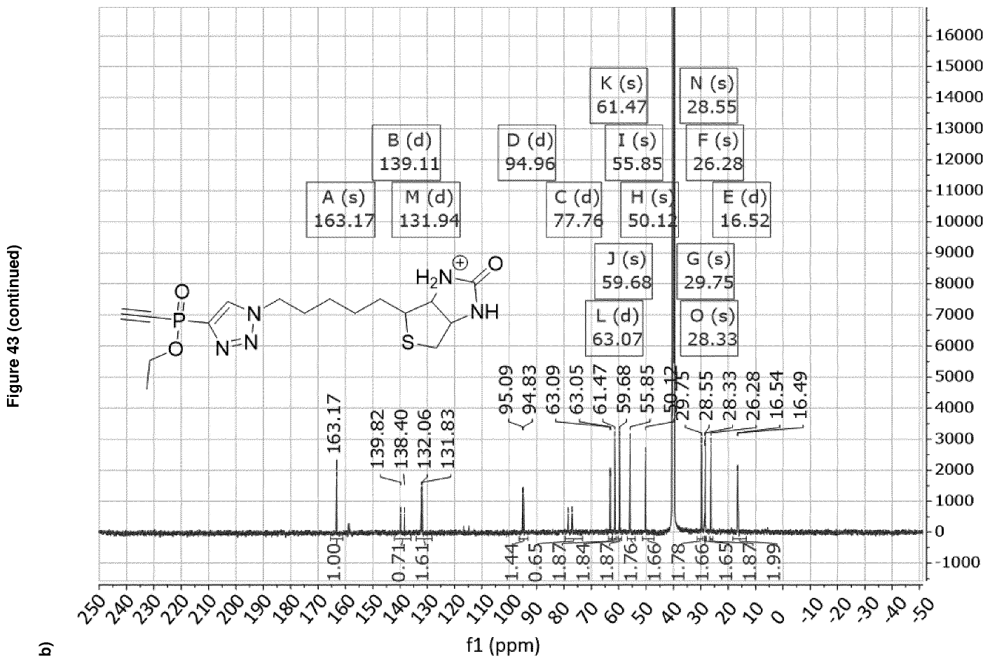

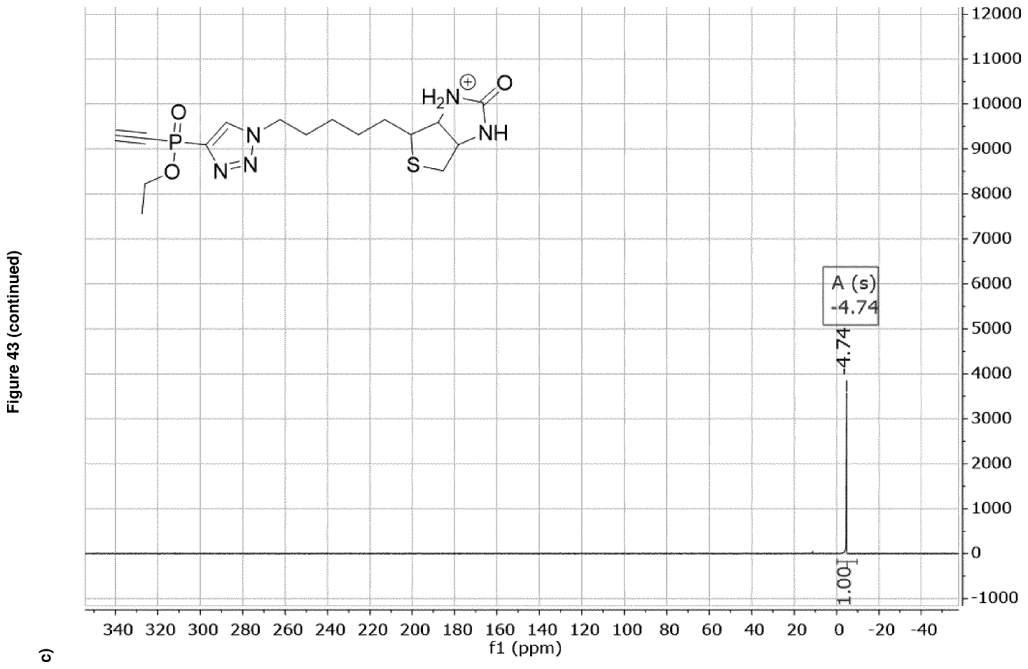

CS292 was synthesized analogously to CS266 from 16 mg Biotin-N₃. (18.4 mg, 74% yield). NMR spectra of CS292 are shown in FIG. 43 (a) ¹H; b)¹³C; c)³¹P).

¹H NMR (600 MHz, DMSO-d₆) δ 8.77 (s, 1H), 4.71 (d, J=11.6 Hz, 1H), 4.45 (t, J=7.1 Hz, 2H), 4.31 (dd, J=7.7, 5.0 Hz, 1H), 4.22-4.08 (m, 3H), 3.16-3.00 (m, 1H), 2.82 (dd, J=12.4, 5.1 Hz, 1H), 2.59 (d, J=12.4 Hz, 1H), 1.96-1.80 (m, 2H), 1.60 (ddd, J=11.8, 8.0, 4.3 Hz, 1H), 1.51-1.08 (m, 8H).

¹C NMR (151 MHz, DMSO-d₆) δ 163.17, 139.11 (d, J=213.4 Hz), 131.94 (d, J=34.0 Hz), 94.96 (d, J=39.3 Hz), 77.76 (d, J=220.2 Hz), 63.07 (d, J=5.9 Hz), 61.47, 59.68, 55.85, 50.12, 29.75, 28.55, 28.33, 26.28, 16.52 (d, J=6.8 Hz).

¹P NMR (243 MHz, DMSO-d₆) δ-4.74.

HRMS for C₁₆H₂₄N₅O₃SP [M+H]⁺ calc.: 398.1410; found: 398.1409

### Diethynyl(Phenyl)Phosphine Oxide (CS267)

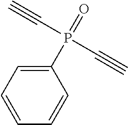

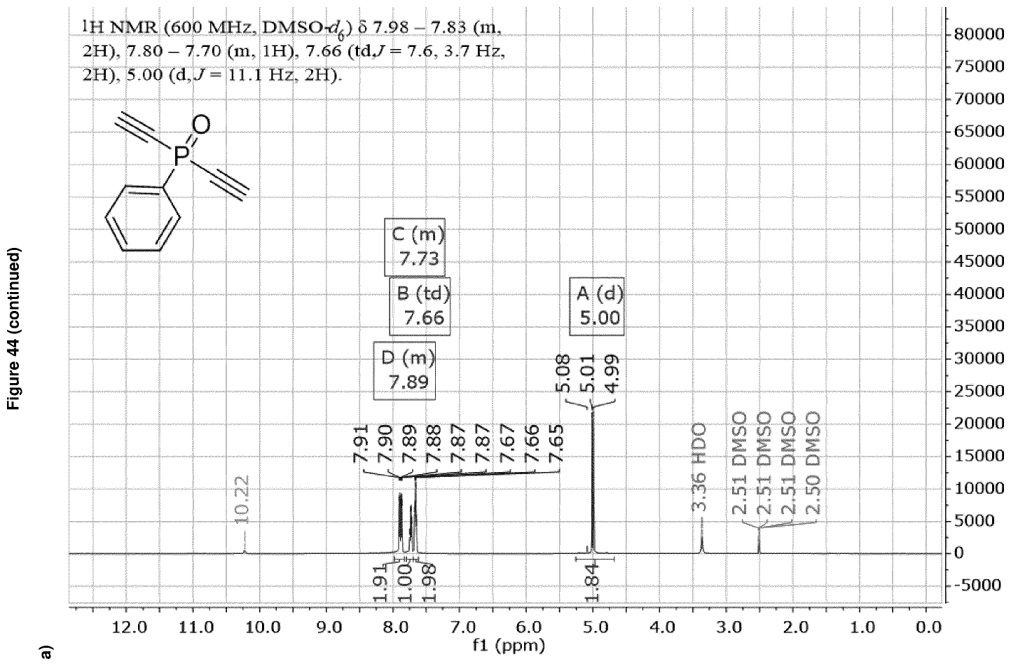

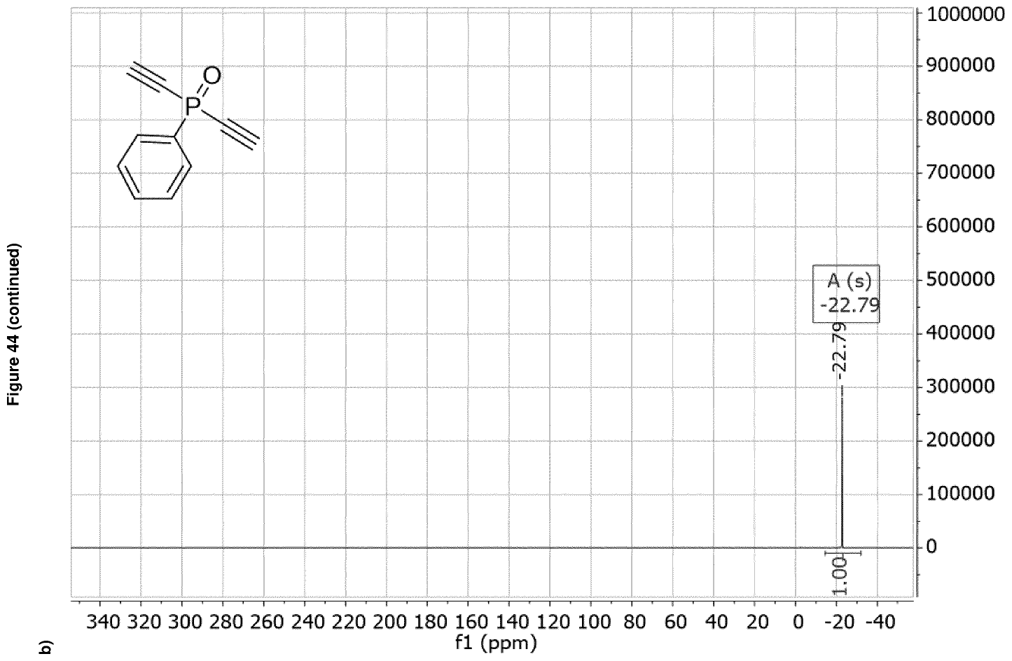

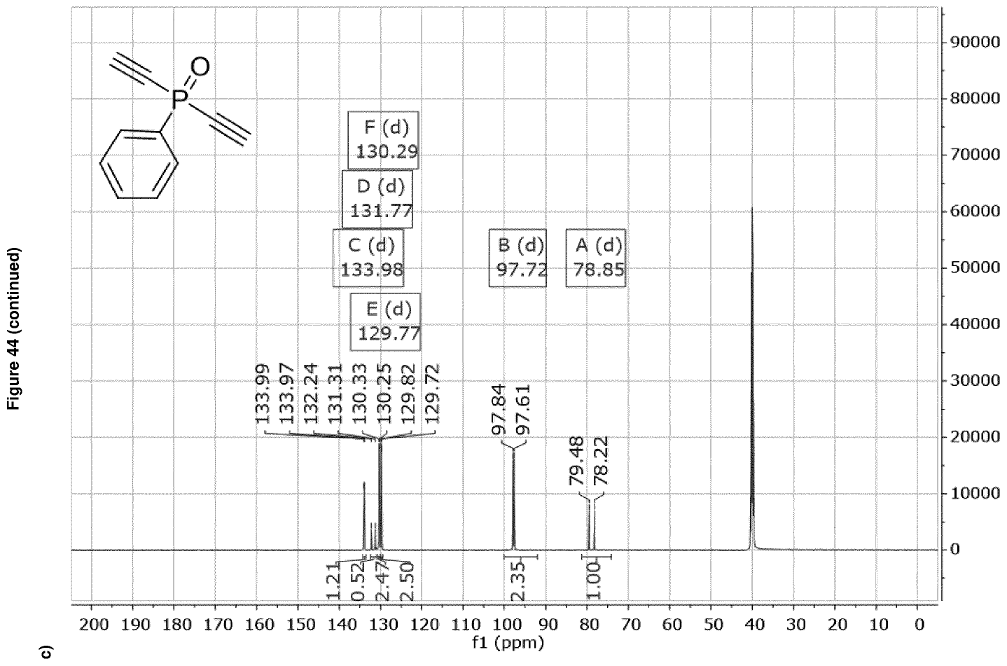

CS267 was synthesized according to ethyl di-ethynyl phosphinate, starting from the commercially available dichloro-phenylphosphine. NMR spectra of CS67 are shown in FIG. 44 (a)¹H; b)³¹P; c)¹³C).

¹H NMR (600 MHz, DMSO-d₆) δ 7.98-7.83 (m, 2H), 7.80-7.70 (m, 1H), 7.66 (td, J=7.6, 3.7 Hz, 2H), 5.00 (d, J=11.1 Hz, 2H).

¹³C NMR (151 MHz, DMSO-d₆) δ 133.98 (d, J=3.2 Hz), 131.77 (d, J=140.6 Hz), 130.29 (d, J=12.7 Hz), 129.77 (d, J=14.8 Hz), 97.72 (d, J=34.0 Hz), 78.85 (d, J=189.8 Hz).

³¹P NMR (243 MHz, DMSO-d₆) δ-22.79.

HRMS for C₁₀H₈OP⁺ [M+H]⁺ calc.: 175.0307; found: 175.0316

### Tri-ethynylphosphinate (CS297)

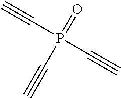

The compound was synthesized according to S. G. A. Van Assema, C. G. J. Tazelaar, G. De Bas Jong, J. H. Van Maarseveen, M. Schakel, M. Lutz, A. L. Spek, J. Chis Slootweg, K. Lammertsma, Phospha-scorpionate complexes by click chemistry using phenyl azide and ethynylphosphine oxides, Organometallics. 27 (2008) 3210-3215. https://doi.org/10.1021/om800127 h. Spectroscopic characterization was in agreement with the literature. (152 mg, 40% yield)

¹H NMR (600 MHz, DMSO-d₆) δ 5.13 (d, J=12.5 Hz, 3H).

¹³C NMR (151 MHz, DMSO-d₆) δ 97.07 (d, J=42.8 Hz), 78.09 (d, J=227.6 Hz).

³¹P-NMR (243 MHz, DMSO-d₆) δ-56.9.

HRMS for C₆H₄OP⁺ [M+H⁺] calc.: 122.9994; found: 123.0009

Bis-ethynyl(ethyl)phosphine oxide (CS297-Sideproduct)

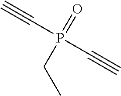

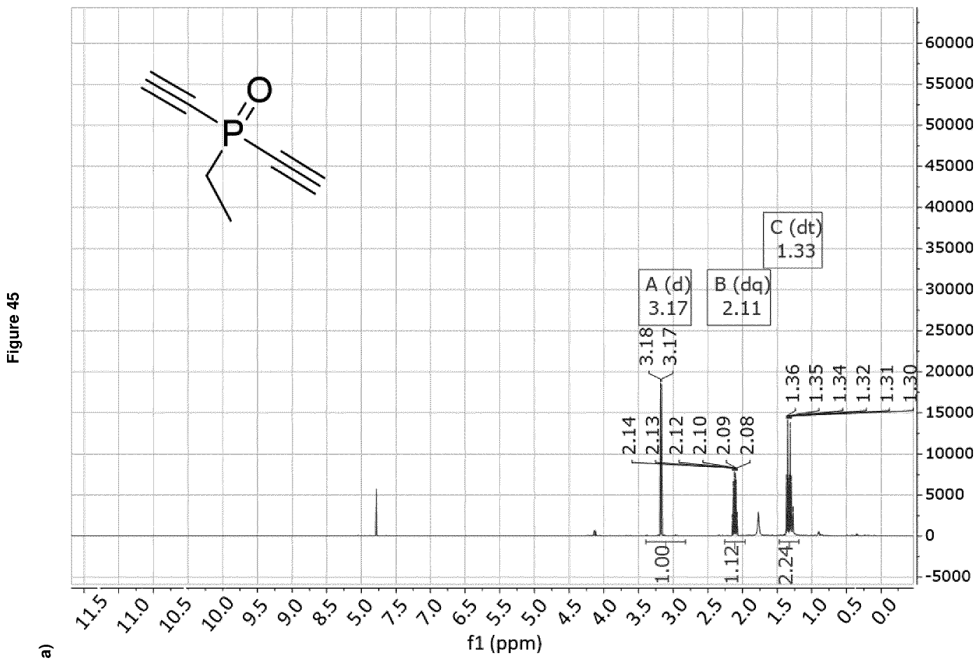

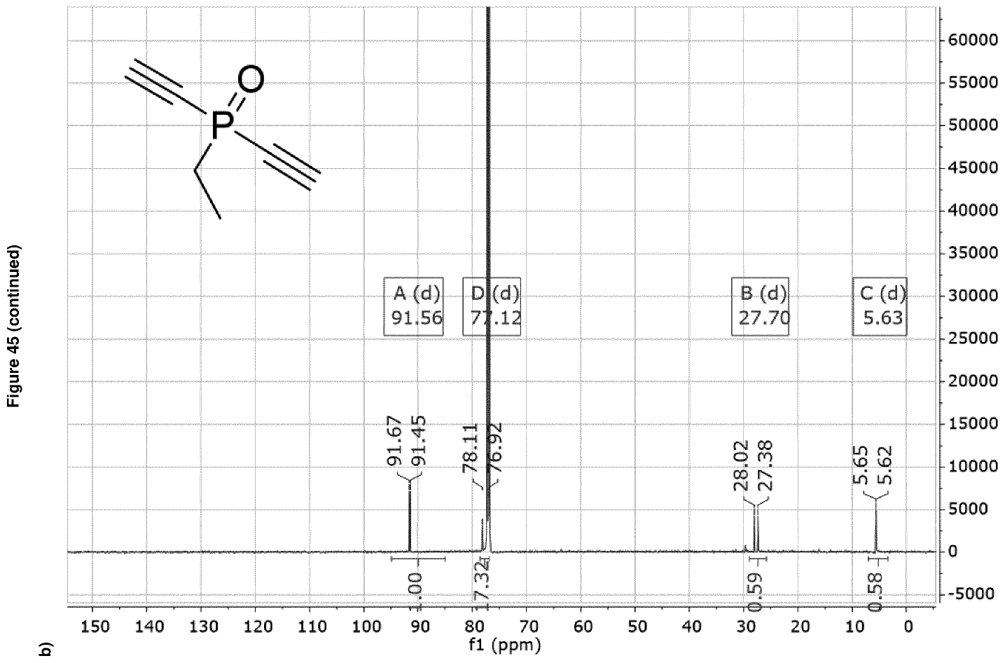

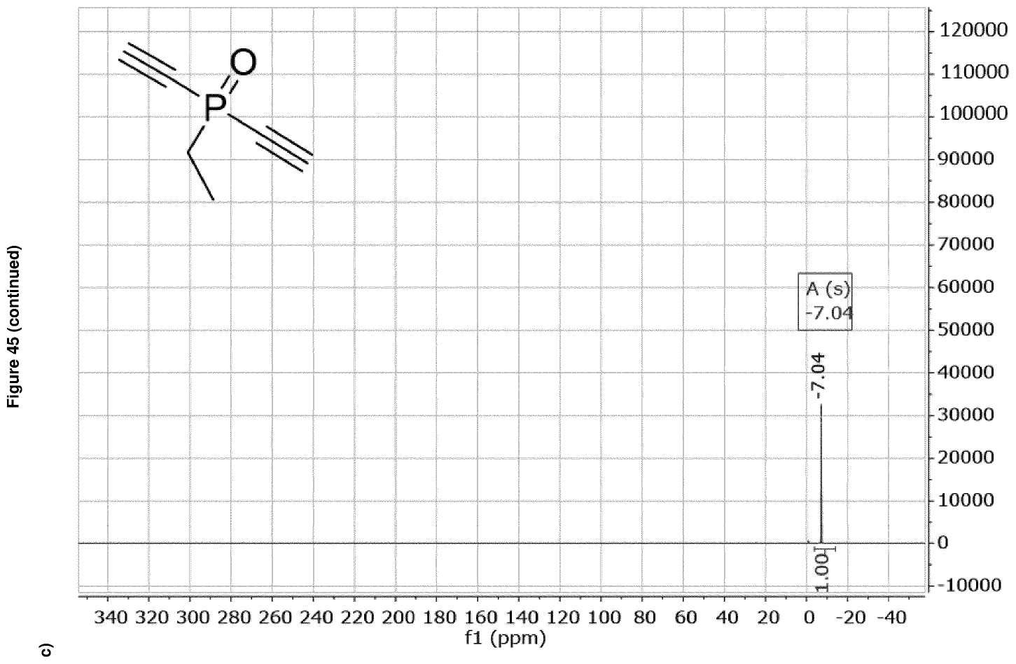

Was isolated as a side product in the synthesis of CS297. (5 mg, 1.3% yield). NMR spectra of CS197 are shown in FIG. 45 (a)¹H; b)¹³C; c)³¹P).

¹H NMR (600 MHz, Chloroform-d) δ 3.17 (d, J=10.5 Hz, 2H), 2.11 (dq, J=15.3, 7.6 Hz, 2H), 1.33 (dt, J=23.7, 7.6 Hz, 3H).

¹³C NMR (151 MHz, Chloroform-d) δ 91.56 (d, J=32.8 Hz), 77.12 (d, J=31.9 Hz), 27.70 (d, J=97.6 Hz), 5.63 (d, J=4.8 Hz).

³¹P NMR (243 MHz, Chloroform-d) δ-7.04.

HRMS for C₆H₈OP⁺ [M+H⁺] calc.: 127.0307; found: 127.0310

### Compound CS298

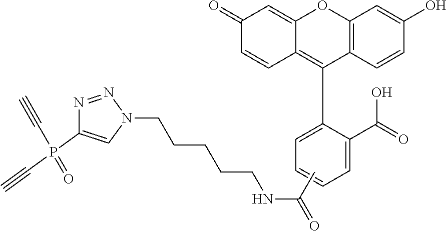

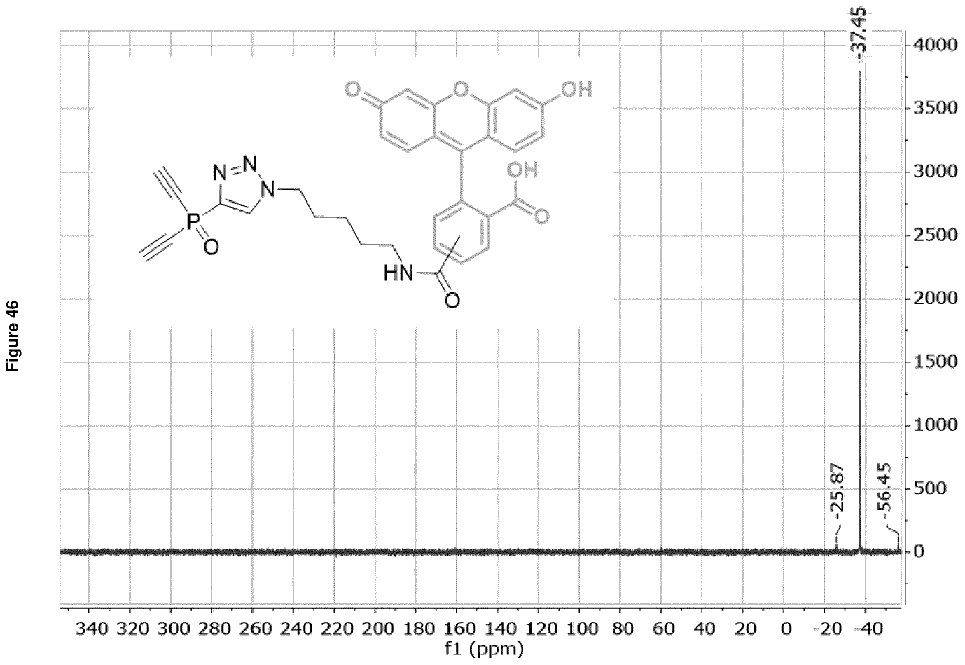

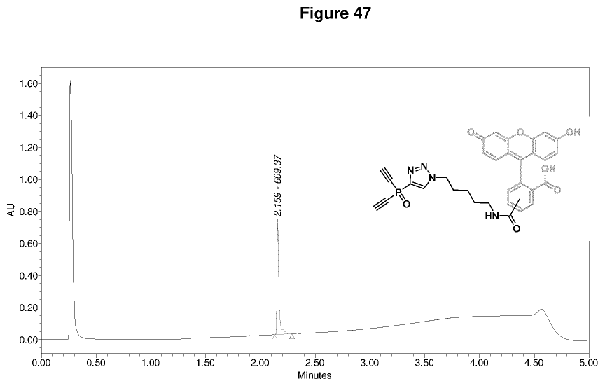

CS298 was synthesized from 4 mg FAM-N₃ (mixture of 5/6-isomers) and 10 eq. ethyl-diethynyl-phosphinate (1) dissolved in 1 ml DMSO/PBS (1:1). The reaction was started by the addition of 3 mg CuBr. After 30 minutes the reaction was stopped by the addition of 4 ml 0.1% aq. TFA and purified via semi-preparative HPLC. (3.2 mg, 62% yield). A ³¹P NMR spectrum of CS298 is shown in FIG. 46. An HPLC chromatogram of CS298 is shown in FIG. 47.

³¹P NMR (243 MHz, DMSO-d₆) δ-37.45.

HRMS for C₃₂H₂₆N₄O₇P⁺ [M+H⁺] calc.: 609.1534; found: 609.1494

### R₁₀-1,2,3-triazol-ethyl-ethynylphosphinate (CS314)

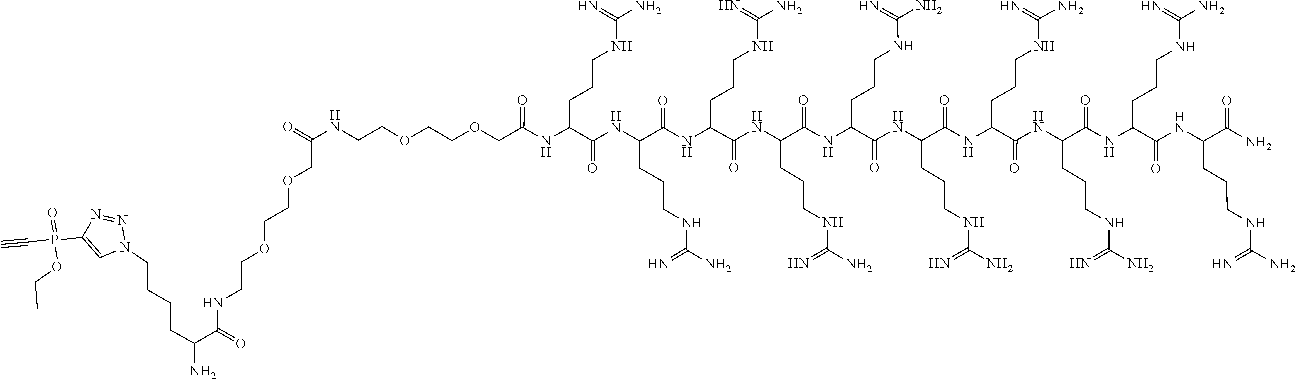 [SMILES: C#CP(=O)(OCC)c1cn(CCCCC(N)C(=O)NCCOCCOCC(=O)NCCOCCOCC(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(=O)NC(CCCNC(=N)N)C(N)=O)nn1]

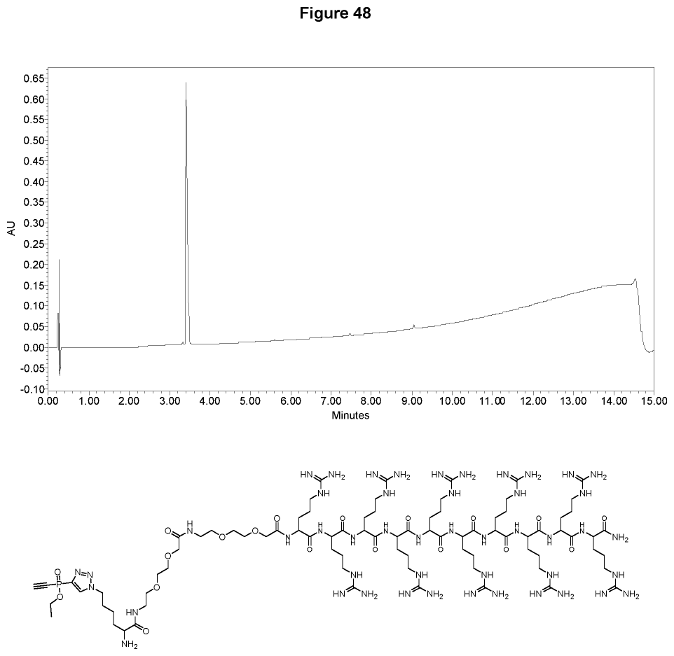

CS314 was synthesized analogously to CS266 from 9 mg of the corresponding azide-substituted peptide. An HPLC chromatogram of of CS314 is shown in FIG. 48.

HRMS for C₈₄H₁₆₆N₄₇O₁₉P⁴⁺ [M+4H⁺] calc.: 542.0796; found: 542.0784

### Compound CS321

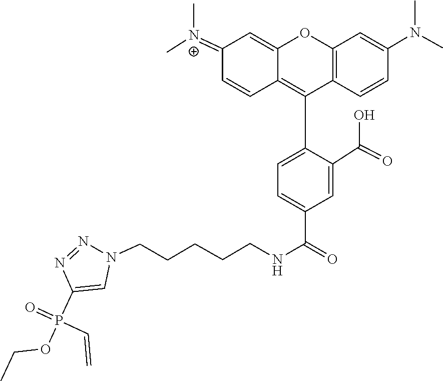 [SMILES: C=CP(=O)(OCC)c1cn(CCCCCNC(=O)c2ccc(-c3c4ccc(=[N+](C)C)cc-4oc4cc(N(C)C)ccc34)c(C(=O)O)c2)nn1]

CS321 was synthesized from ethyl-vinyl-ethynyl-phosphinate (1.2 eq.) and TAMRA-N₃ (5 mg 1 eq., mixture of 5/6-isomers) dissolved in 400 μl DMSO/PBS (1:1) at room temperature. The reaction was started by the addition of 5 mg CuBr. After 30 minutes, the reaction was stopped by the addition of 3 ml 0.1% aq. TFA and CS321 was purified via semi-preparative HPLC. (5.4 mg, 86% yield)

¹H NMR (600 MHz, Acetonitrile-d₃) δ 8.64 (d, J=10.8 Hz, 1H), 8.26-8.17 (m, 2H), 7.59 (t, J=6.3 Hz, 1H), 7.43 (dd, J=7.0, 4.8 Hz, 1H), 7.12 (t, J=10.2 Hz, 2H), 6.94 (dt, J=13.0, 6.4 Hz, 2H), 6.84 (dd, J=10.3, 2.9 Hz, 2H), 6.55-6.07 (m, 3H), 4.47 (q, J=6.1, 4.9 Hz, 2H), 4.18-3.88 (m, 2H), 3.44 (p, J=6.1 Hz, 2H), 3.26 (d, J=10.9 Hz, 12H), 2.02 (dd, J=14.8, 7.2 Hz, 4H), 1.69 (p, J=6.5, 5.6 Hz, 2H), 1.43 (q, J=7.6 Hz, 2H), 1.28 (td, J=7.0, 4.6 Hz, 3H).

¹³C NMR (189 MHz, Acetonitrile-d₃) δ 165.91, 165.40, 157.31 (d, J=7.1 Hz), 139.28 (d, J=176.2 Hz), 136.74, 136.57, 135.16 (d, J=1.8 Hz), 131.72, 131.11, 130.84, 130.72, 130.57, 129.99, 129.74 (d, J=144.9 Hz), 129.25, 114.17, 96.24, 61.33 (d, J=6.0 Hz), 50.05, 39.35, 29.43, 28.43, 23.56, 15.78 (d, J=6.4 Hz).

³¹P NMR (243 MHz, Acetonitrile-d₃) δ 20.02

HRMS for C₃₆H₄₂N₆O₆P⁺ [M+H]⁺ calc.: 685.2898; found: 685.2901

### Compound CS327

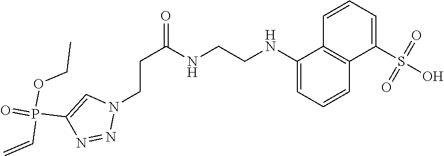 [SMILES: C=CP(=O)(OCC)c1cn(CCC(=O)NCCNc2cccc3c(S(=O)(=O)O)cccc23)nn1]

CS321 was synthesized according to CS321 from 15 mg EDANS-N₃. (14.3 mg, 68% yield)

¹H NMR (600 MHz, DMSO-d₆) δ 8.57 (s, 1H), 8.41 (d, J=8.7 Hz, 1H), 8.27 (t, J=5.7 Hz, 1H), 8.08 (d, J=8.5 Hz, 1H), 7.99 (dd, J=7.2, 1.1 Hz, 1H), 7.46-7.42 (m, 1H), 7.38 (t, J=8.1 Hz, 1H), 7.23-7.01 (m, 0H), 6.96-6.85 (m, 1H), 6.46 (ddd, J=24.4, 18.6, 12.7 Hz, 1H), 6.32-6.08 (m, 2H), 4.68 (t, J=6.8 Hz, 2H), 4.07-3.85 (m, 2H), 3.40 (q, J=6.4 Hz, 2H), 3.31 (t, J=6.6 Hz, 2H), 2.82 (t, J=6.8 Hz, 2H), 1.21 (t, J=7.0 Hz, 3H).

¹³C NMR (189 MHz, DMSO-d₆) δ 170.04, 144.62, 141.10, 139.14 (d, J=174.8 Hz), 135.88, 131.99 (d, J=28.6 Hz), 130.64 (d, J=25.6 Hz), 129.97, 126.42, 125.11, 124.20 (d, J=154.0 Hz), 123.17, 119.44, 107.61, 61.28 (d, J=5.8 Hz), 46.49, 45.10, 37.60, 35.73, 16.73 (d, J=6.1 Hz).

³¹P NMR (243 MHz, DMSO-d₆) δ 19.60.

HRMS calculated for C₂₁H₂₆N₅O₆PS⁺ [M+H] calc.: 508.1414; found: 508.1416

### Compound CS145

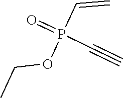

1 mmol diethyl-vinylphosphonate (153 μl, 1 eq.) was dissolved in 4 ml dry DCM under argon. 350 μl oxalylchloride were added in one portion and the reaction was stirred overnight at 30° C. followed by 30 min reflux. Volatiles were removed under high vacuum and the residue (ethyl vinylphosphonochloridate, ³¹P-NMR: 26.95 ppm) was dissolved in dry THF to form a 1 M solution. After cooling to 0° C., 2.2 ml of ethynyl-MgBr (0.5 M in THF, 1.1 eq.) were added and the reaction was allowed to warm to room temperature. The reaction was partitioned between DCM and water and the aqueous layer was extracted two additional times with DCM. Combined organic fractions were dried (MgSO₄) filtered and evaporated. The residue was used without further purification. (71 mg, 50%)

¹H NMR (600 MHz, Chloroform-d) δ 6.44 (ddd, J=27.2, 16.0, 4.1 Hz, 1H), 6.37-6.14 (m, 2H), 4.35-4.15 (m, 2H), 3.04 (d, J=10.7 Hz, 1H), 1.41 (t, J=7.1 Hz, 3H).

¹³C NMR (151 MHz, Chloroform-d) δ 136.01 (d, J=2.6 Hz), 128.78 (d, J=160.0 Hz), 89.72 (d, J=36.8 Hz), 76.95 (d, J=202.8 Hz), 62.40 (d, J=6.6 Hz), 16.22 (d, J=7.0 Hz).

³¹P NMR (243 MHz, Chloroform-d) δ 6.54.

### 2-(6-phenyl-1,2,4,5-tetrazin-3-yl)ethan-1-ol(CS333)

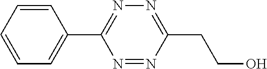 [SMILES: OCCc1nnc(-c2ccccc2)nn1]

Synthesized according to Mao et al. (W. Mao, W. Shi, J. Li, D. Su, X. Wang, L. Zhang, L. Pan, X. Wu, H. Wu, Organocatalytic and Scalable Syntheses of Unsymmetrical 1,2,4,5-Tetrazines by Thiol-Containing Promotors, Angew. Chemie Int. Ed. 58 (2019) 1106-1109. https://doi.org/10.1002/anie.) from benzonitrile and hydroxypropionitrile.

¹H NMR (600 MHz, DMSO-d₆) δ 8.48 (dd, J=7.5, 2.6 Hz, 2H), 7.68 (ddt, J=17.1, 8.8, 4.6 Hz, 3H), 4.84 (td, J=5.6, 2.7 Hz, 1H), 4.04 (qd, J=6.1, 2.7 Hz, 2H), 3.46 (td, J=6.4, 2.8 Hz, 2H).

¹³C NMR (151 MHz, DMSO-d₆) δ 168.68, 164.01, 132.95, 132.31, 130.04, 129.90, 127.90, 59.79, 38.62.

HRMS for C₁₀H₁₁N₄O⁺ [M+H]⁺ calc.: 203.0927; found: 203.0937

### 3-(2-azidoethyl)-6-phenyl-1,2,4,5-tetrazine (CS347)

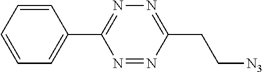 [SMILES: [N-]=[N+]=NCCc1nnc(-c2ccccc2)nn1]

50 mg CS333 (0.25 mmol, 1 eq.) were dissolved in 2 ml dry DCM and cooled to 0° C. 1.2 eq. triethylamine were added, followed by the dropwise addition of 1.2 eq. methanesulfonylchloride (dissolved in 2 ml dry DCM). After 1 h at room temperature, the reaction was extracted three times with H₂O, dried over MgSO₄, filtered and evaporated to dryness. The residue was dissolved in 3 ml DMSO, 5 eq. NaN₃ were added, and the reaction was stirred overnight at room temperature. Salts were precipitated with Et₂O (30 ml) and filtered over cellite. The filtrate was evaporated to dryness and CS347 was used without further purification. (pink powder; 51 mg, 91%)

¹H NMR (600 MHz, Chloroform-d) δ 8.70-8.55 (m, 1H), 7.74-7.55 (m, 2H), 4.05 (t, J=6.8 Hz, 1H), 3.66 (t, J=6.7 Hz, 1H).

¹³C NMR (151 MHz, Chloroform-d) δ 167.15, 164.63, 132.86, 131.55, 129.31, 128.13, 48.76, 34.46.

HRMS for C₁₀H₁₀N₇⁺ [M+H]⁺ calc.: 228.0992; found: 228.0999

### Ethyl ethynyl(1-(2-(6-phenyl-1,2,4,5-tetrazin-3-yl)ethyl)-1H-1,2,3-triazol-4-yl) phosphinate (CS380)

CS380 was synthesized analogously to CS266 from 10 mg CS347. (8.4 mg, 52% yield).

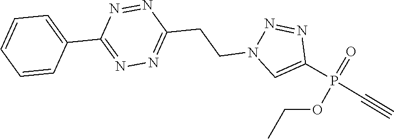 [SMILES: C#CP(=O)(OCC)c1cn(CCc2nnc(-c3ccccc3)nn2)nn1]

¹H NMR (600 MHz, DMSO-d₆) δ 8.87 (s, 1H), 8.59-8.43 (m, 2H), 7.84-7.60 (m, 3H), 5.12 (t, J=6.9 Hz, 2H), 4.72 (d, J=11.6 Hz, 1H), 4.15 (dqd, J=9.6, 7.0, 2.8 Hz, 2H), 4.01 (t, J=6.9 Hz, 2H), 1.29 (t, J=7.0 Hz, 3H).

³¹P NMR (243 MHz, Acetonitrile-d₃) δ-5.21.

¹³C NMR (151 MHz, DMSO-d₆) δ 167.22, 164.12, 139.10 (d, J=212.9 Hz), 133.20, 132.60 (d, J=34.2 Hz), 132.09, 129.99, 128.03, 95.06 (d, J=39.6 Hz), 77.66 (d, J=221.1 Hz), 63.11 (d, J=5.9 Hz), 47.79, 34.87, 16.49 (d, J=6.9 Hz).

HRMS for C₁₆H₁₆N₇O₂P⁺ [2M+Na]⁺ calc.: 761.2096; found: 761.2093.

### 3-(2-(2-(2-(2-hydroxyethoxy)ethoxy)ethoxy)ethoxy)benzonitrile (CS337)

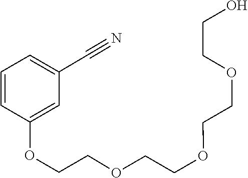 [SMILES: N#Cc1cccc(OCCOCCOCCOCCO)c1]

10 mmol 3-cyanophenol were dissolved in 10 ml acetone. 2 eq. K₂CO₃ were added followed by 1 eq. tetraethyleneglycol-tosylate. The reaction was refluxed for 5 h until no starting material was left. The mixture was dilluted with water (100 ml) and extracted with EtOAc (3×50 ml). Combined organic layers were dried (MgSO₄), filtered and evaporated. The residue was purified over silica (EtOAc/MeOH; 10:1) to obtain the desired product as a colourless oil. (1.8 g, 61%)

¹H NMR (600 MHz, Chloroform-d) δ 7.36 (t, J=7.9 Hz, 1H), 7.24 (dt, J=7.5, 1.2 Hz, 1H), 7.19-7.13 (m, 2H), 4.17-4.13 (m, 2H), 3.89-3.83 (m, 2H), 3.75-3.63 (m, 11H), 3.63-3.57 (m, 2H).

¹³C NMR (151 MHz, Chloroform-d) δ 158.90, 130.32, 124.65, 119.93, 118.68, 117.67, 113.12, 72.47, 70.84, 70.64, 70.55, 70.30, 69.48, 67.86, 61.68.

HRMS for C₁₅H₂₂NO₅⁺ [M+H]⁺ calc.: 296.1492; found: 296.1497

### 2-(2-(2-(2-(3-(6-phenyl-1,2,4,5-tetrazin-3-yl)phenoxy)ethoxy)ethoxy)ethoxy) ethan-1-ol (CS338)

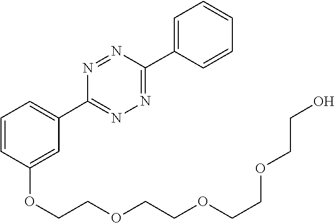 [SMILES: OCCOCCOCCOCCOc1cccc(-c2nnc(-c3ccccc3)nn2)c1]

Synthesized according to Mao et al. (W. Mao, W. Shi, J. Li, D. Su, X. Wang, L. Zhang, L. Pan, X. Wu, H. Wu, Organocatalytic and Scalable Syntheses of Unsymmetrical 1,2,4,5-Tetrazines by Thiol-Containing Promotors, Angew. Chemie Int. Ed. 58 (2019) 1106-1109. https://doi.org/10.1002/anie.) from CS337 and benzonitrile.

¹H NMR (600 MHz, Acetonitrile-d₃) δ 8.70-8.56 (m, 2H), 8.24 (dd, J=7.8, 1.3 Hz, 1H), 8.18 (dd, J=2.8, 1.4 Hz, 1H), 7.79-7.65 (m, 3H), 7.61 (td, J=8.0, 1.3 Hz, 1H), 7.29 (dd, J=8.3, 2.6 Hz, 1H), 4.38-4.25 (m, 2H), 3.88 (td, J=4.3, 1.2 Hz, 2H), 3.77-3.68 (m, 2H), 3.66-3.58 (m, 8H), 3.52 (td, J=4.8, 1.3 Hz, 2H).

¹³C NMR (151 MHz, Acetonitrile-d₃) δ 165.45, 165.24, 161.07, 135.00, 134.07, 133.61, 132.10, 130.84, 129.14, 121.73, 120.72, 114.32, 73.67, 71.77, 71.58, 71.54, 71.39, 70.65, 69.26, 62.33.

HRMS for C₂₂H₂₇N₄O₅⁺ [M+H]* calc.: 427.1976; found: 427.1981

### Compound CS344A

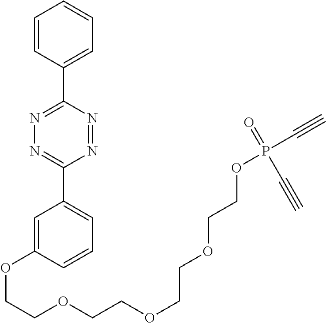 [SMILES: C#CP(=O)(C#C)OCCOCCOCCOCCOc1cccc(-c2nnc(-c3ccccc3)nn2)c1]

Synthesized according to general Procedure A from 20 mg CS338. (16 mg, 67%)

¹H NMR (600 MHz, DMSO-d₆) δ 8.64-8.49 (m, 2H), 8.26-8.11 (m, 1H), 8.07 (d, J=2.0 Hz, 1H), 7.72 (qd, J=8.8, 7.8, 3.8 Hz, 4H), 7.62 (t, J=8.0 Hz, 1H), 7.32 (dd, J=8.3, 2.6 Hz, 1H), 4.86 (d, J=12.7 Hz, 2H), 4.27 (dd, J=5.7, 3.5 Hz, 2H), 4.23-4.12 (m, 2H), 3.83 (dd, J=5.5, 3.6 Hz, 2H), 3.68 (t, J=4.5 Hz, 3H), 3.65 (dd, J=5.9, 3.6 Hz, 2H), 3.61-3.56 (m, 7H).

³¹P NMR (243 MHz, DMSO-d₆) δ-22.95.

¹³C NMR (151 MHz, DMSO-d₆) δ 165.26, 165.04, 161.06, 135.05, 134.56, 133.71, 132.67, 131.39, 129.47, 121.92, 121.04, 114.63, 96.52 (d, J=45.7 Hz), 78.53 (d, J=252.9 Hz), 71.83, 71.72, 71.67, 71.61, 70.81, 70.75, 69.39, 68.00 (d, J=6.1 Hz).

HRMS for C₂₆H₂₆N₄O₄P⁺ [M+H]⁺ calc.: 523.1741; found: 523.1744

### Diethynyl(1-(2-(6-phenyl-1,2,4,5-tetrazin-3-yl)ethyl)-1H-1,2,3-triazol-4-yl)phosphine oxide (CS350)

CS350 was synthesized according to CS298 from 15 mg CS347. (7 mg, 30%).

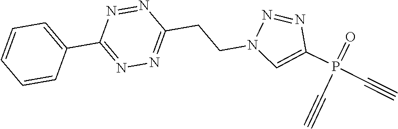 [SMILES: C#CP(=O)(C#C)c1cn(CCc2nnc(-c3ccccc3)nn2)nn1]

¹H NMR (600 MHz, Chloroform-d) δ 8.70-8.53 (m, 2H), 8.31 (s, 1H), 7.79-7.56 (m, 4H), 5.25 (t, J=6.8 Hz, 2H), 4.13 (t, J=6.8 Hz, 2H), 3.33 (d, J=11.7 Hz, 2H).

³¹P NMR (243 MHz, Chloroform-d) δ-35.18.

¹³C NMR (151 MHz, Acetonitrile-d₃) δ168.19, 165.86, 141.57 (d, J=191.3 Hz), 134.12, 133.46, 132.90 (d, J=35.7 Hz), 130.82, 129.20, 95.64 (d, J=39.1 Hz), 78.85 (d, J=205.2 Hz), 49.07, 35.91.

HRMS for C₁₆H₁₃N₇OP⁺ [M+H]⁺ calc.: 350.0914; found: 350.0918

### Exo-5-Norbornenecarboxylicacid NHS-Ester

Synthesized according to P. Werther, J. S. Möhler, R. Wombacher, Chem. Eur. J. 2017, 23, 18216.

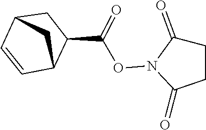 [SMILES: O=C(ON1C(=O)CCC1=O)[C@@H]1C[C@H]2C=C[C@@H]1C2]

¹H NMR (600 MHz, DMSO-d₆) δ 6.25 (dd, J=5.6, 3.0 Hz, 1H), 6.20 (dd, J=5.7, 3.1 Hz, 1H), 3.20-3.11 (m, 1H), 3.00 (dq, J=3.6, 1.8 Hz, 1H), 2.83 (s, 4H), 2.55 (ddd, J=9.1, 4.4, 1.3 Hz, 1H), 1.90 (dt, J=11.8, 3.9 Hz, 1H), 1.51 (ddd, J=11.7, 9.0, 2.4 Hz, 1H), 1.38 (qt, J=8.8, 1.9 Hz, 2H).

¹³C NMR (151 MHz, DMSO-d₆) δ 172.05, 170.74, 138.69, 135.67, 47.02, 46.42, 41.68, 30.97, 25.93 (One signal overlaps with the solvent-peak).

### Ethyl norbornene-PEG₇-triazol-ethynyl-phosphinate (CS390)

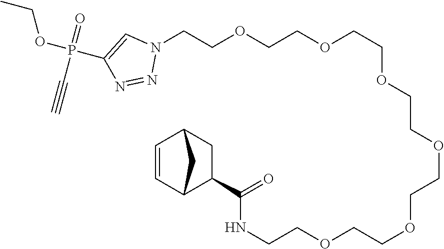 [SMILES: C#CP(=O)(OCC)c1cn(CCOCCOCCOCCOCCOCCOCCNC(=O)[C@@H]2C[C@H]3C=C[C@@H]2C3)nn1]

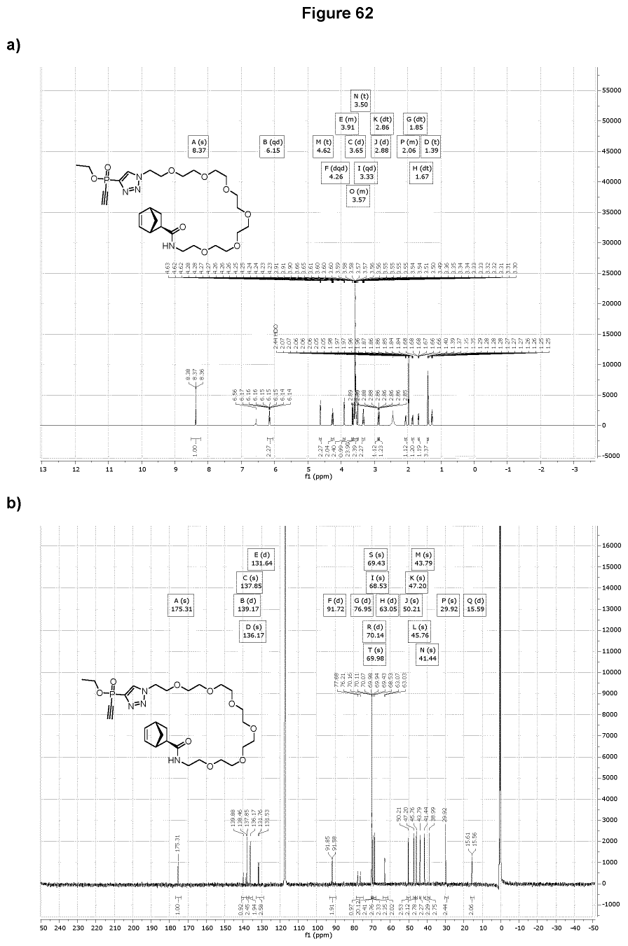

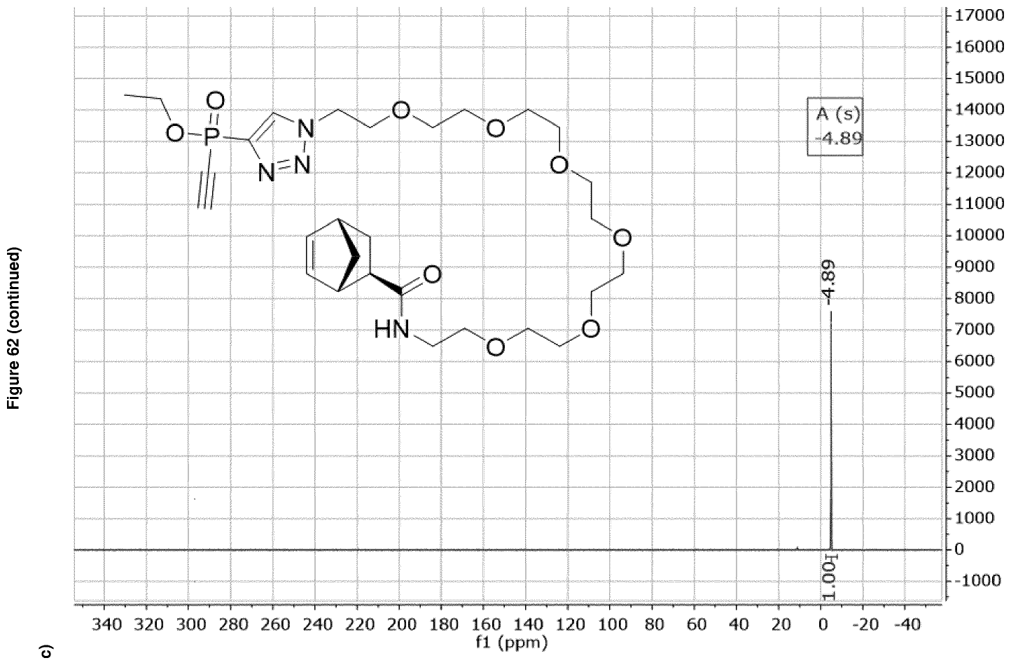

H₂N-PEG₇N₃ (11 mg, 0.02 mmol) was dissolved in 100 μl DMSO and 200 μl PBS (pH 7.4). 1.2 eq. exo-5-norbornencarboxylic acid NHS-ester were added and allowed to react for 1 h at room temperature. After full consumption of the amine (UPLC-MS) 5 eq. phosphinate 1 and 20 mol % CuBr were added. After 30 minutes, the reaction was stopped by the addition of 3 ml 0.1% aq. TFA and 7 was purified via semi-preparative HPLC. (11.1 mg, 59% yield). NMR spectra of CS390 are shown in FIG. 62 (a) ¹H; b) 13C; c)³¹P)

¹H NMR (600 MHz, Acetonitrile-d₃) δ 8.37 (s, 1H), 6.15 (qd, J=5.6, 2.8 Hz, 2H), 4.62 (t, J=5.1 Hz, 2H), 4.26 (dqd, J=9.0, 7.0, 2.1 Hz, 2H), 3.94-3.87 (m, 2H), 3.65 (d, J=11.5 Hz, 1H), 3.63-3.52 (m, 24H), 3.50 (t, J=5.6 Hz, 2H), 3.33 (qd, J=5.6, 2.3 Hz, 2H), 2.88 (d, J=3.1 Hz, 1H), 2.86 (dt, J=2.8, 1.3 Hz, 1H), 2.09-2.03 (m, 1H), 1.85 (dt, J=11.4, 4.0 Hz, 1H), 1.67 (dt, J=8.0, 1.5 Hz, 1H), 1.39 (t, J=7.1 Hz, 3H).

¹³C NMR (151 MHz, Acetonitrile-d₃) δ 175.31, 139.17 (d, J=214.8 Hz), 137.85, 136.17, 131.64 (d, J=33.8 Hz), 91.72 (d, J=40.6 Hz), 76.95 (d, J=221.0 Hz), 70.14 (d, J=7.4 Hz), 69.98, 69.94, 69.43, 68.53, 63.05 (d, J=6.1 Hz), 50.21, 47.20, 45.76, 43.79, 41.44, 38.99, 29.92, 15.59 (d, J=7.1 Hz).

³¹P NMR (243 MHz, Acetonitrile-d₃) δ-4.89.

HRMS for C₂₈H₄₆N₄O₉P⁺ [M+H⁺] calc.: 613.2997; found: 613.3019.

### 5/6-Carboxyfluorescein-Azide

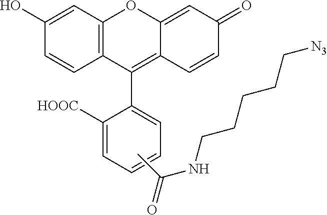

HRMS for C₂₆H₂₃N₄O₆P [M+H]⁺ calc.: 487.1612; found: 687.1613.

5/6-Carboxyfluorescein-1,2,3-triazol-ethyl-ethynylphosphinate (CS375)

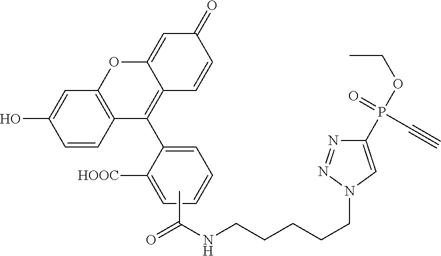

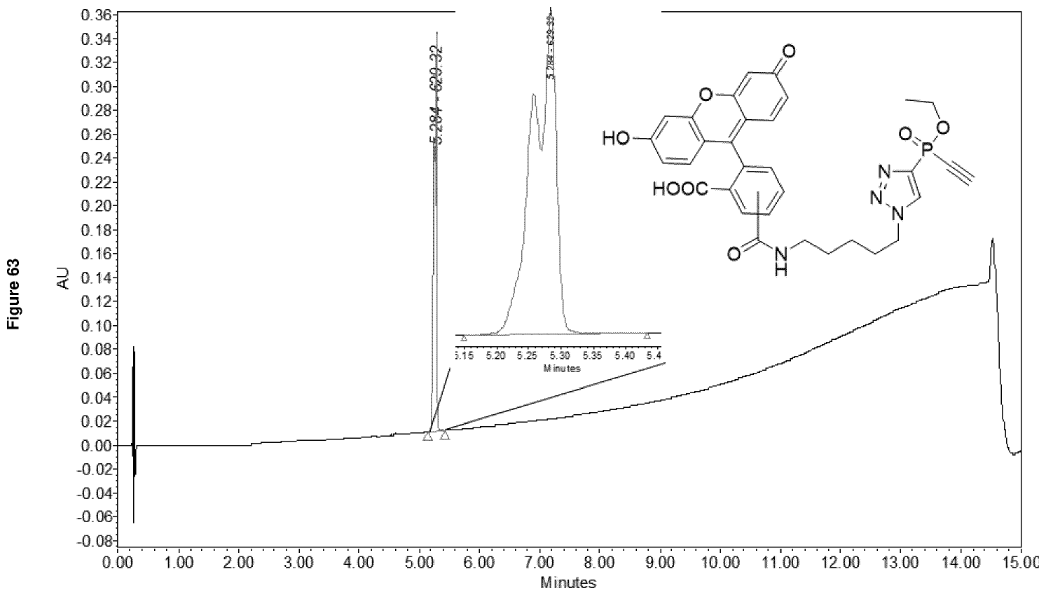

CS375 was synthesized analogously to CS298 from 3 mg 5/6-Carboxyfluoresceine-N₃ and phosphinate 1. (2.7 mg, 70%). An HPLC chromatogram of CS375 is shown in FIG. 63.

HRMS for C₃₂H₃₀N₄O₈P [M+H]⁺ calc.: 629.1796; found: 629.1769

### Desthiobiotin NHS-Ester

Desthiobiotin (214 mg, 1 mmol) was dissolved in 2 ml dry DMF. EDC-HCl (1.1 eq.) and NHS (1.1 eq.) were added, and the reaction was stirred overnight at room temperature. The reaction mixture was diluted with 50 ml 2M HCl and extracted with EtOAc (3×30 ml). The combined organic layers were dried (MgSO₄), filtered and evaporated to obtain desthiobiotin as a white solid (280 mg, 90%).

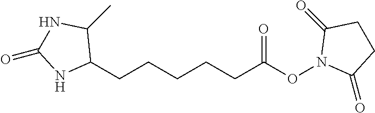 [SMILES: CC1NC(=O)NC1CCCCCC(=O)ON1C(=O)CCC1=O]

¹H NMR (600 MHz, DMSO-d₆) δ 3.66-3.59 (m, 1H), 3.50 (td, J=7.9, 4.7 Hz, 1H), 2.82 (d, J=6.2 Hz, 5H), 2.67 (t, J=7.3 Hz, 2H), 1.63 (p, J=7.2 Hz, 2H), 1.36 (dddd, J=20.5, 18.3, 9.7, 4.7 Hz, 5H), 1.22 (qd, J=9.3, 7.6, 5.0 Hz, 1H), 0.97 (d, J=6.4 Hz, 3H).

¹³C NMR (151 MHz, DMSO-d₆) δ 170.72, 169.44, 163.30, 55.37, 50.69, 30.57, 29.84, 28.48, 25.90, 25.76, 24.66, 15.94.

### Desthiobiotin-N₃ (CS374)

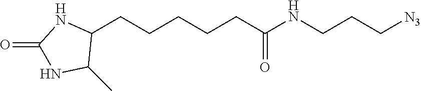 [SMILES: CC1NC(=O)NC1CCCCCC(=O)NCCCN=[N+]=[N-]]

20 mg 3-azidopropanamine was dissolved in 1 ml dry DMF and 40 μl diisopropylamine. 60 mg (0.95 eq.) Desthiobiotin-NHS-ester were added and the reaction was stirred for 2 h at room temperature. The product was isolated via preparative HPLC. (62 mg, 84%).

¹H NMR (600 MHz, DMSO-d₆) δ 7.82 (t, J=5.6 Hz, 1H), 3.61 (dd, J=7.6, 6.3 Hz, 1H), 3.48 (td, J=8.0, 4.7 Hz, 1H), 3.34 (t, J=6.8 Hz, 2H), 3.09 (q, J=6.5 Hz, 2H), 2.05 (t, J=7.4 Hz, 2H), 1.64 (p, J=6.8 Hz, 2H), 1.49 (p, J=7.4 Hz, 2H), 1.41-1.12 (m, 6H), 0.96 (d, J=6.4 Hz, 3H).

¹³C NMR (151 MHz, DMSO-d₆) δ 172.62, 163.27, 55.45, 50.70, 48.91, 36.21, 35.79, 29.97, 29.18, 28.93, 26.01, 25.63, 15.93.

HRMS C₁₃H₂₅N₆O₂⁺ [M+H⁺] calc.: 297.2034; found: 297.2036.

### Desthiobiotin-1,2,3-triazol-ethyl-ethynylphosphinate (CS418)

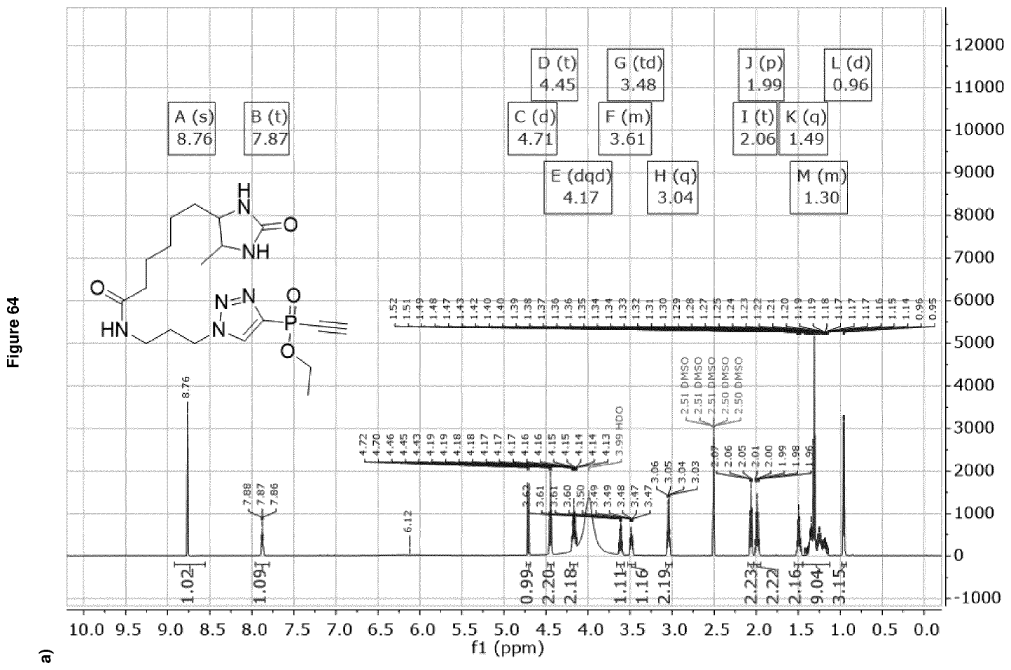

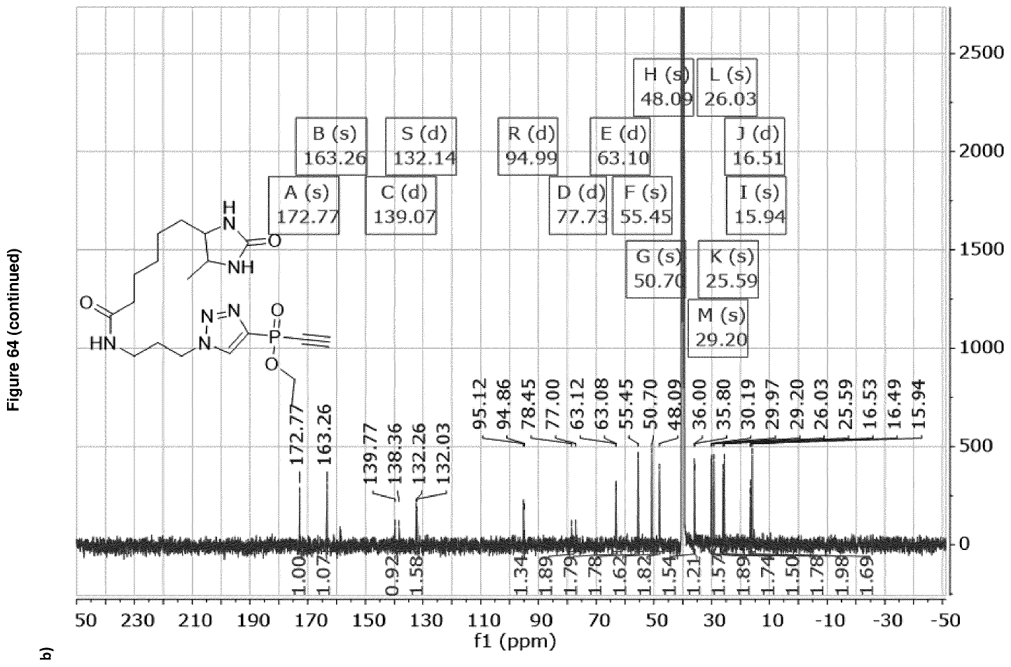

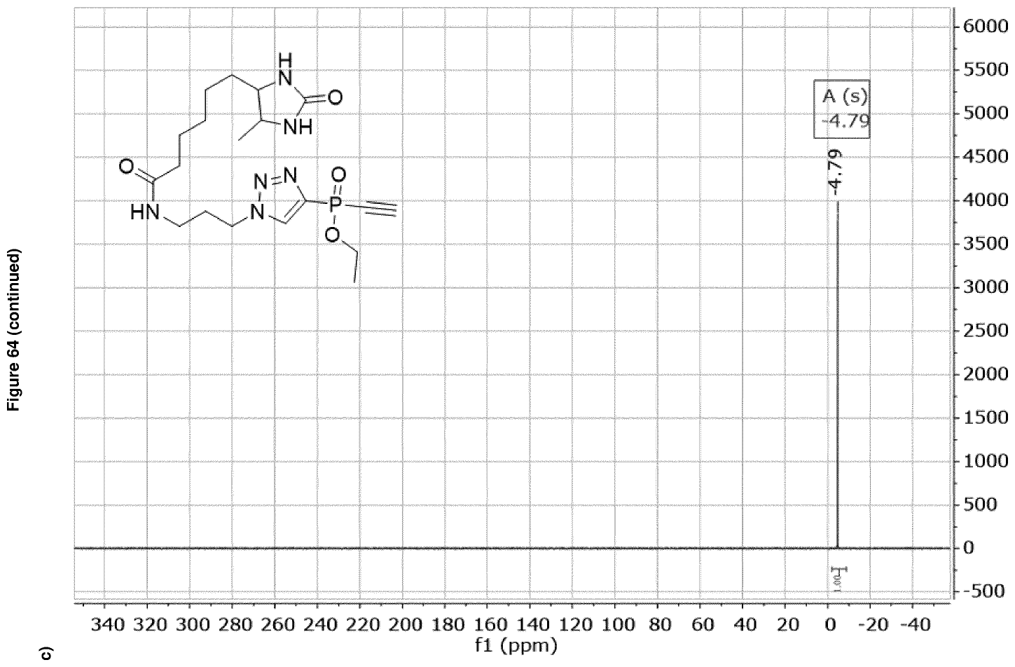

CS418 was synthesized analogously to CS298 from 10 mg CS374 and phosphinate 1. (9.27 mg, 63%). NMR spectra of CS418 are shown in FIG. 64 (a) ¹H; b)¹³C; c)³¹P).

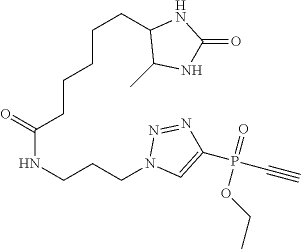 [SMILES: C#CP(=O)(OCC)c1cn(CCCNC(=O)CCCCCC2NC(=O)NC2C)nn1]

¹H NMR (600 MHz, DMSO-d₆) δ 8.76 (s, 1H), 7.87 (t, J=5.7 Hz, 1H), 4.71 (d, J=11.6 Hz, 1H), 4.45 (t, J=7.0 Hz, 2H), 4.17 (dqd, J=9.4, 7.0, 2.5 Hz, 2H), 3.66-3.57 (m, 1H), 3.48 (td, J=7.9, 4.6 Hz, 1H), 3.04 (q, J=6.5 Hz, 2H), 2.06 (t, J=7.5 Hz, 2H), 1.99 (p, J=6.9 Hz, 2H), 1.49 (q, J=7.5 Hz, 2H), 1.44-1.12 (m, 9H), 0.96 (d, J=6.4 Hz, 3H).

¹³C NMR (151 MHz, DMSO-d₆) δ 172.77, 163.26, 139.07 (d, J=213.1 Hz), 132.14 (d, J=34.3 Hz), 94.99 (d, J=39.6 Hz), 77.73 (d, J=219.3 Hz), 63.10 (d, J=5.7 Hz), 55.45, 50.70, 48.09, 36.00, 35.80, 30.19, 29.97, 29.20, 26.03, 25.59, 16.51 (d, J=6.6 Hz), 15.94.

³¹P NMR (243 MHz, DMSO-d₆) δ-4.79.

HRMS C₁₉H₃₂N₆O₄P⁺ [M+H⁺] calc.: 439.2217; found: 439.2231

### Cy5-1,2,3-triazol-ethyl-ethynylphosphinate (CS450)

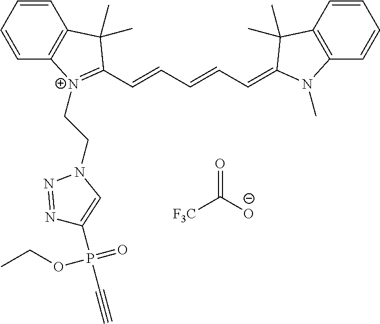 [SMILES: C#CP(=O)(OCC)c1cn(CC[N+]2=C(/C=C/C=C/C=C3/N(C)c4ccccc4C3(C)C)C(C)(C)c3ccccc32)nn1 ; O=C([O-])C(F)(F)F]

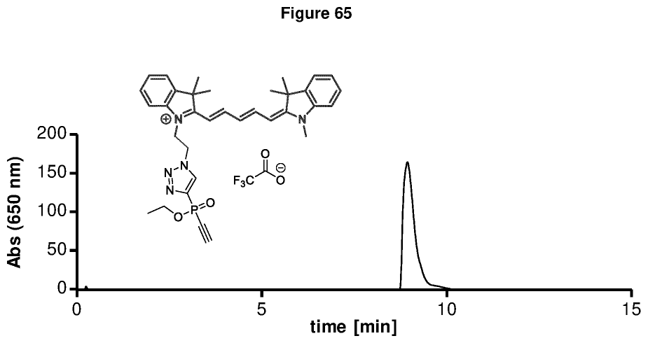

CS450 was synthesized analogously to CS298 from 5 mg Cy5-Azide and phosphinate 1. (4.8 mg, 78% yield). An HPLC chromatogram of CS450 is shown in FIG. 65.

HRMS for C₃₄H₃₉N₅O₂P⁺ [M+H]⁺ calc.: 580.2836; found: 580.2866.

### 4-(6-methyl-1,2,4,5-tetrazin-3-yl)phenol (CS342)

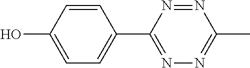

CS342 was synthesized according to Yang et al. (Yang, J., Karver, M. R., Li, W., Sahu, S. and Devaraj, N. K. (2012), Metal-Catalyzed One-Pot Synthesis of Tetrazines Directly from Aliphatic Nitriles and Hydrazine. Angew. Chem. Int. Ed., 51: 5222-5225.)

¹H NMR (600 MHz, Acetonitrile-d₃) δ 8.51-8.30 (m, 2H), 7.67 (s, 1H), 7.18-6.88 (m, 2H), 2.99 (s, 3H).

¹³C NMR (151 MHz, Acetonitrile-d₃) δ 166.77, 163.66, 160.90, 129.55 (2C), 123.89, 116.12 (2C), 20.27.

### 3-(4-(2-(2-(2-azidoethoxy)ethoxy)ethoxy)phenyl)-6-methyl-1,2,4,5-tetrazine (CS414)

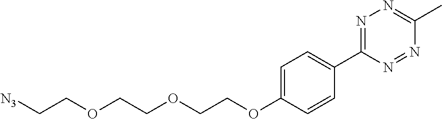 [SMILES: Cc1nnc(-c2ccc(OCCOCCOCCN=[N+]=[N-])cc2)nn1]

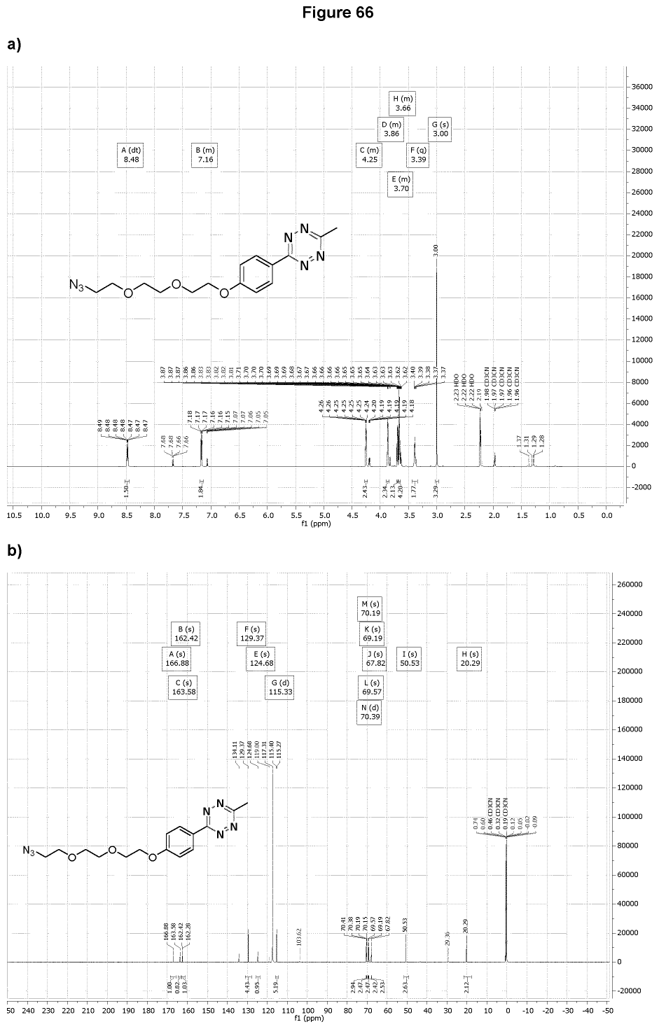

0.7 mmol CS342 were dissolved in 15 ml DMF/MeCN (5:1). 2 eq. K₂CO₃ and 1.2 eq. Tosyl-PEG₃-N₃ were added and the reaction was stirred at 60° C. for 2 h. The mixture was extracted from 1M HCl (100 ml) and EtOAc (3×50 ml), the combined organic extracts were dried over MgSO₄, filtered and evaporated. The obtained crude was purified over Silica (Hexane/EtOAC 2:1) to obtain 183 mg of CS414 as a pink solid. (76% yield). NMR spectra of CS414 are shown in FIG. 66 (a)¹H; b)¹³C).

¹H NMR (600 MHz, acetonitrile-d₃) δ 8.48 (dt, J=8.9, 2.0 Hz, 2H), 7.19-7.13 (m, 2H), 4.28-4.22 (m, 2H), 3.89-3.84 (m, 2H), 3.71-3.68 (m, 2H), 3.68-3.64 (m, 4H), 3.39 (q, J=4.4, 3.9 Hz, 2H), 3.00 (s, 3H).

¹³C NMR (151 MHz, acetonitrile-d₃) δ 166.88, 163.58, 162.42, 129.37, 124.68, 115.33 (d, J=19.0 Hz), 70.39 (d, J=4.2 Hz), 70.19, 69.57, 69.19, 67.82, 50.53, 20.29.

### Tetrazine-PEG3-Triazolyl-Phosphinoxide (CS415)

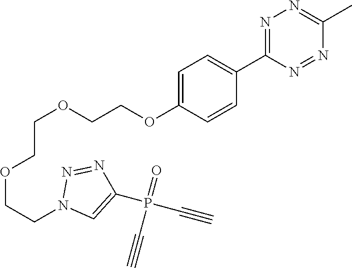 [SMILES: C#CP(=O)(C#C)c1cn(CCOCCOCCOc2ccc(-c3nnc(C)nn3)cc2)nn1]

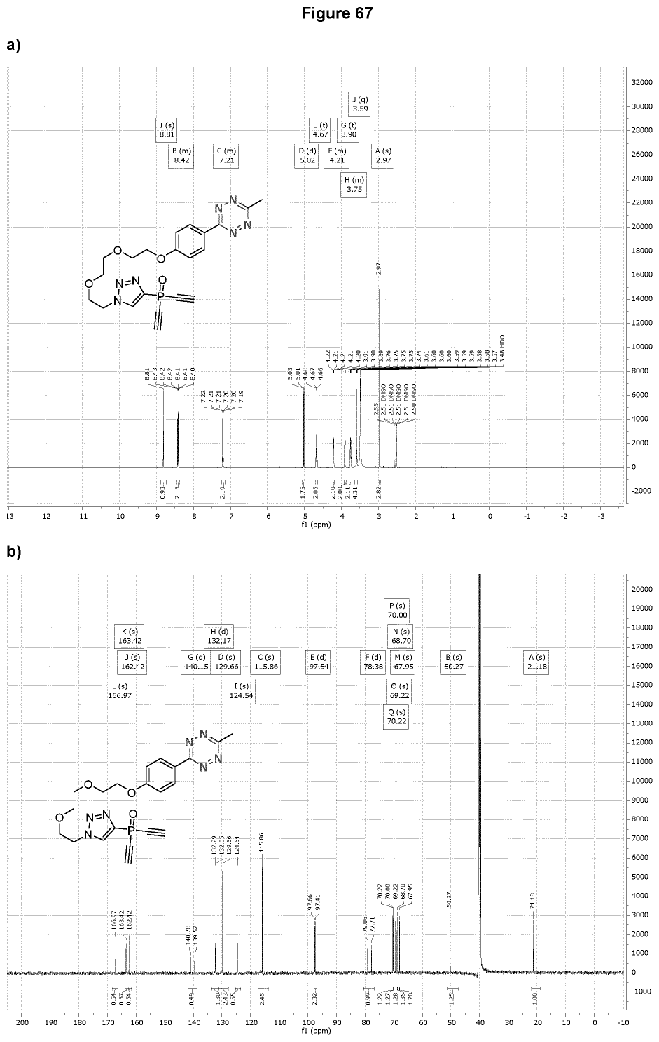

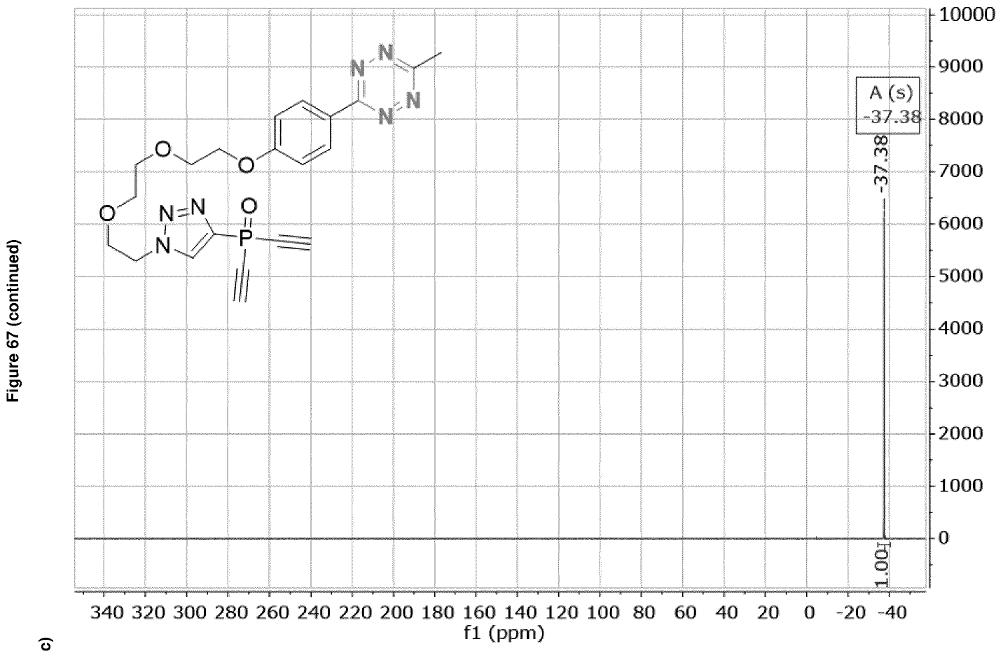

CS415 was synthesized according to CS298 from 17 mg CS414. (13.07 mg, 56% yield). NMR spectra of CS415 are shown in FIG. 67 (a)¹H; b) ¹³C); c)³¹P).

¹H NMR (600 MHz, DMSO-d₆) δ 8.81 (s, 1H), 8.45-8.38 (m, 2H), 7.23-7.15 (m, 2H), 5.02 (d, J=11.6 Hz, 2H), 4.67 (t, J=5.2 Hz, 2H), 4.23-4.19 (m, 2H), 3.90 (t, J=5.2 Hz, 2H), 3.80-3.72 (m, 2H), 3.59 (q, J=1.5 Hz, 4H), 2.97 (s, 3H).

¹C NMR (151 MHz, DMSO-d₆) δ 166.97, 163.42, 162.42, 140.15 (d, J=190.3 Hz), 132.17 (d, J=36.0 Hz), 129.66, 124.54, 115.86, 97.54 (d, J=37.5 Hz), 78.38 (d, J=203.2 Hz), 70.22, 70.00, 69.22, 68.70, 67.95, 50.27, 21.18. ³¹P NMR (243 MHz, DMSO-d₆) δ-37.38.

HRMS for C₂₁H₂₃N₇O₄P⁺ [M+H]⁺ calc.: 468.1544 Da; found: 468.1564 Da

### N₃-PEG₄-Val-Cit-PAB-MMAE

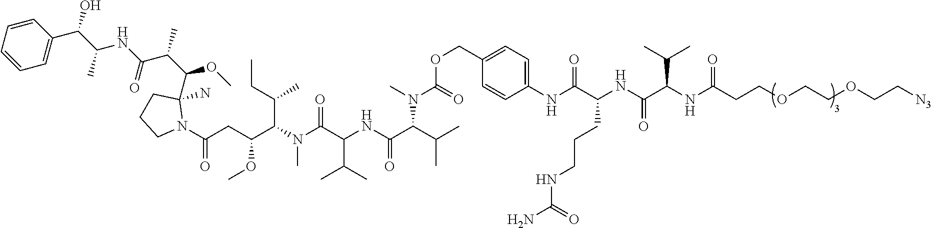

A 5 ml flask was charged with 37.9 mg Val-Cit-PAB-MMAE (synthesized according to Matos et al.(M. J. Matos, C. D. Navo, T. Hakala, X. Ferhati, A. Guerreiro, D. Hartmann, B. Bemardim, K. L. Saar, I. Comparñón, F. Corzana, T. P. J. Knowles, G. Jiménez-Osés, G. J. L. Bemardes, Angew. Chem. Int. Ed. 2019, 58, 6640.)) in 0.3 ml DMSO. 1.2 eq. N₃-PEG₄-NHS-ester (Jena Bioscience) and 2 eq. DIPEA were added. The reaction was allowed to proceed for 4 h at 50° C. and the product was purified via semi-preparative HPLC (37.6 mg, 71% yield).

HRMS for C₅₉H₁₁₅N₁₃O₁₇²⁺ [M+2H]²⁺ calc: 698.9261 Da; found: 698.9261 Da

### (PEG₄-Val-Cit-PAB-MMAE)-triazolyl-mPEG₄-ethynyl phosphinate (CSDrug1)

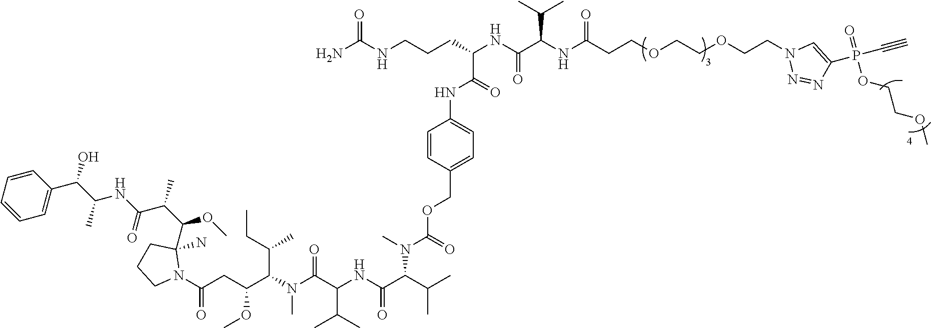

CSDrug1 was synthesized accordingly to CS298 from 2.55 μmol N₃-PEG₄-Val-Cit-PAB-MMAE and 12.75 μmol phosphinate 3. The product was purified via semi-preparative HPLC. (2.57 mg, 59% yield)

HRMS for C₈₂H₁₃₆N₁₃O₂₃P²⁺ [M+2H]²⁺ calc: 850.9799 Da; found: 850.9773 Da

### (PEG₄-Val-Cit-PAB-MMAE)-triazolyl diethynyl phosphinoxide

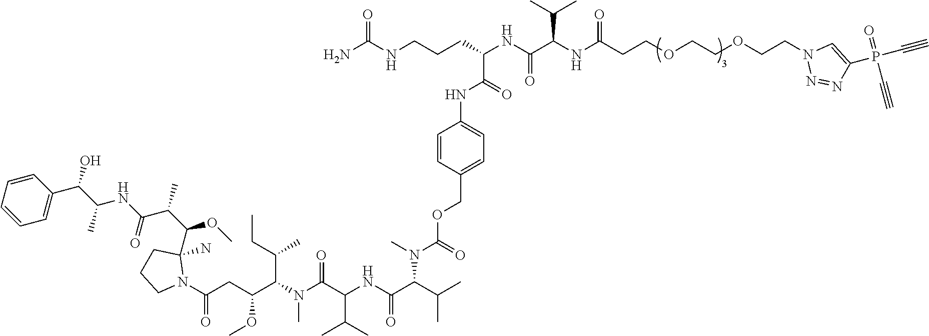

The compound was synthesized accordingly to CS298 from 2.55 μmol N₃-PEG₄Val-Cit-PAB-MMAE and 6.2 mg phosphinoxid CS297. The product was purified via semi-preparative HPLC. (0.62 mg, 16% yield).

HRMS for C₇₅H₁₁₈N₁₃O₁₈P⁺ [M+2H]²⁺ calc: 759.9222 Da; found: 759.9233 Da.

### CONCLUSION

In conclusion, the present invention provides a novel technique for conjugating compounds comprising a thiol group and rebridging of disulfide groups based on bis-reactive unsaturated phosphorus (V) compounds. The bis-reactive unsaturated phosphorus(V) compounds show excellent reactivity and selectivity towards sulfhydryl groups, e.g. in aqueous systems, which allows the easy and modular generation of conjugates with complex molecules. Moreover, the obtained conjugates exhibit outstanding stability, e.g. in the presence of an excess of small thiols, in the presence of human serum and under conditions inside living cells. Antibodies rebridged with the phosphorus(V) compounds can be further modified with a functional molecule, e.g. a fluorophore, and remain their target selectivity. This simple and straightforward strategy for modification of compounds comprising a sulfhydryl group and antibodies will help to develop new tools for biological and biopharmaceutical applications.

### REFERENCES

[1] E. A. Hoyt, P. M. S. D. Cal, B. L. Oliveira, G. J. L. Bemardes, Nat. Rev. Chem. 2019, 3, 147-171.
[2] D. Schumacher, C. P. R. Hackenberger, Curr. Opin. Chem. Biol. 2014, 22, 62-69.
[3] S. B. Gunnoo, A. Madder, ChemBioChem 2016, 17, 529-553.
[4] P. Ochtrop, C. P. R. Hackenberger, Curr. Opin. Chem. Biol. 2020, 58, 28-36.
[5] S. Jia, D. He, C. J. Chang, 2019, DOI 10.1021/jacs.8b11912.
[6] J. C. Vantourout, S. Rao Adusumalli, K. W. Knouse, D. T. Flood, A. Ramirez, N. M. Padial, A. Istrate, K. Maziarz, J. N. deGruyter, R. R. Merchant, J. X. Qiao, M. A. Schmidt, M. J. Deery, M. D. Eastgate, P. E. Dawson, alo J. L Bemardes, P. S. Baran, J. Am. Chem. Soc 2020, 142, 46.
[7] S. Sato, M. Matsumura, T. Kadonosono, S. Abe, T. Ueno, H. Ueda, H. Nakamura, Bioconjug. Chem. 2020, 31, 1417-1424.
[8] M. T. Taylor, J. E. Nelson, M. G. Suero, M. J. Gaunt, Nature 2018, 562, 563-568.
[9] D. Hwang, N. Nilchan, A. R. Nanna, X. Li, M. D. Cameron, W. R. Roush, H. J. Park, C. Rader, Cell Chem. Biol. 2019, 26, 1229-1239.e9.
[10] K. Lang, J. W. Chin, ACS Chem. Biol. 2014, 9, 16-20.
[11] X. Chen, Y. W. Wu, Org. Biomol. Chem. 2016, 14, 5417-5439.
[12] B. Q. Shen, K. Xu, L. Liu, H. Raab, S. Bhakta, M. Kenrick, K. L. Parsons-Reponte, J. Tien, S. F. Yu, E. Mai, D. Li, J. Tibbitts, J. Baudys, O. M. Saad, S. J. Scales, P. J. McDonald, P. E. Hass, C. Eigenbrot, T. Nguyen, W. A. Solis, R. N. Fuji, K. M. Flagella, D. Patel, S. D. Spencer, L. A. Khawli, A. Ebens, W. L. Wong, R. Vandlen, S. Kaur, M. X. Sliwkowski, R. H. Scheller, P. Polakis, J. R. Junutula, Nat. Biotechnol. 2012, 30, 184-189.
[13] M. E. B. Smith, F. F. Schumacher, C. P. Ryan, L. M. Tedaldi, D. Papaioannou, G. Waksman, S. Caddick, J. R. Baker, J. Am. Chem. Soc. 2010, 132, 1960-1965.
[14] A. L. Baumann, S. Schwagerus, K. Broi, K. Kemnitz-Hassanin, C. E. Stieger, N. Trieloff, P. Schmieder, C. P. R. Hackenberger, J. Am. Chem. Soc. 2020, 142, 9544-9552.
[15] Y. Zhang, C. Zang, G. An, M. Shang, Z. Cui, G. Chen, Z. Xi, C. Zhou, Nat. Commun. 2020, 11, 1-10.
[16] C. Canovas, M. Moreau, C. Bernhard, A. Oudot, M. Guillemin, F. Denat, V. Goncalves, Angew. Chemie Int. Ed. 2018, 57, 10646-10650.
[17] T. Wang, A. Riegger, M. Lamla, S. Wiese, P. Oeckl, M. Otto, Y. Wu, S. Fischer, H. Barth, S. L. Kuan, T. Weil, Chem. Sci. 2016, 7, 3234-3239.
[18] D. L. Paterson, J. U. Flanagan, P. R. Shepherd, P. W. R. Harris, M. A. Brimble, Chem. A Eur. J. 2020, 26, 10826-10833.
[19] S. J. Walsh, S. Omarjee, W. R. J. D. Galloway, T. T.-L. Kwan, H. F. Sore, J. S. Parker, M. Hyvönen, J. S. Carroll, D. R. Spring, Chem. Sci. 2019, 10, 694-700.
[20] M. E. B. Smith, F. F. Schumacher, C. P. Ryan, L. M. Tedaldi, D. Papaioannou, G. Waksman, S. Caddick, J. R. Baker, J. Am. Chem. Soc. 2010, 132, 1960-1965.
[21] S. Sun, P. Akkapeddi, M. C. Marques, N. Martinez-Sáez, V. M. Torres, C. Cordeiro, O. Boutureira, G. J. L. Bemardes, Org. Biomol. Chem. 2019, 17, 2005-2012.
[22] C. Bahou, E. A. Love, S. Leonard, R. J. Spears, A. Maruani, K. Armour, J. R. Baker, V. Chudasama, Bioconjug. Chem. 2019, 30, 1048-1054.
[23] J. P. M. Nunes, M. Morais, V. Vassileva, E. Robinson, V. S. Rajkumar, M. E. B. Smith, R. B. Pedley, S. Caddick, J. R. Baker, V. Chudasama, Chem. Commun. 2015, 51, 10624-10627.
[24] G. Badescu, P. Bryant, M. Bird, K. Henseleit, J. Swierkosz, V. Parekh, R. Tommasi, E. Pawlisz, K. Jurlewicz, M. Farys, N. Camper, X. Sheng, M. Fisher, R. Grygorash, A. Kyle, A. Abhilash, M. Frigerio, J. Edwards, A. Godwin, Bioconjug. Chem. 2014, 25, 1124-1136.
[25] M. Kasper, M. Glanz, A. Stengl, M. Penkert, S. Klenk, T. Sauer, D. Schumacher, J. Helma, E. Krause, M. C. Cardoso, H. Leonhardt, C. P. R. Hackenberger, Angew. Chemie Int. Ed. 2019, 58, 11625-11630.
[26] M. A. Kasper, M. Glanz, A. Oder, P. Schmieder, J. P. Von Kries, C. P. R. Hackenberger, Chem. Sci. 2019, 10, 6322-6329.
[27] M. Kasper, A. Stengl, P. Ochtrop, M. Gerlach, T. Stoschek, D. Schumacher, J. Helma, M. Penkert, E. Krause, H. Leonhardt, C. P. R. Hackenberger, Angew. Chemie Int. Ed. 2019, 58, 11631-11636.
[28] M. Kasper, M. Gerlach, A. F. L. Schneider, C. Groneberg, P. Ochtrop, S. Boldt, D. Schumacher, J. Helma, H. Leonhardt, M. Christmann, C. P. R. Hackenberger, ChemBioChem 2020, 21, 113-119.
[29] J. S. Harvey, G. T. Giuffredi, V. Gouvemeur, Org. Lett. 2010, 12, 1236-1239.
[30] S. G. A. Van Assema, C. G. J. Tazelaar, G. De Bas Jong, J. H. Van Maarseveen, M.
Schakel, M. Lutz, A. L. Spek, J. Chris Slootweg, K. Lammertsma, Organometallics 2008, 27, 3210-3215.
[31] S. G. A. van Assema, G. B. de Jong, A. W. Ehlers, F. J. J. de Kanter, M. Schakel, A. L. Spek, M. Lutz, K. Lammertsma, European J. Org. Chem. 2007, 2007, 2405-2412.
[32] H.-Y. Shiu, T.-C. Chan, C.-M. Ho, Y. Liu, M.-K. Wong, C.-M. Che, Chem.-A Eur. J. 2009, 15, 3839-3850.
[33] C. P. R. Schneider, Anselm F. L.; Kithil, Marina; Cardoso, M. Cristina; Lehmann, Martin; Hackenberger, 2021, submitted Manuscript.
[34] Z. L. Chen, J. M. Meng, Y. Cao, J. L. Yin, R. Q. Fang, S. B. Fan, C. Liu, W. F. Zeng, Y. H. Ding, D. Tan, L. Wu, W. J. Zhou, H. Chi, R. X. Sun, M. Q. Dong, S. M. He, Nat. Commun. 2019, 10, DOI 10.1038/s41467-019-11337-z.
[35] Ochtrop, P. & Hackenberger, C. P. R. Recent advances of thiol-selective bioconjugation reactions. Current Opinion in Chemical Biology vol. 58 28-36 (2020).
[36] Hacker, S. M. et al. Global profiling of lysine reactivity and ligandability in the human proteome. Nat. Chem. 9, 1181-1190 (2017).
[37] Dovgan, I. et al. Arginine-selective bioconjugation with 4-azidophenyl glyoxal: Application to the single and dual functionalisation of native antibodies. Org. Biomol. Chem. 16, 1305-1311 (2018).
[38] Jia, S., He, D. & Chang, C. J. Bioinspired Thiophosphorodichloridate Reagents for Chemoselective Histidine Bioconjugation. (2019) doi:10.1021/jacs.8b11912.
[39] Vantourout, J. C. et al. Serine-Selective Bioconjugation. J. Am. Chem. Soc 142, 46 (2020).
[40] Tower, S. J., Hetcher, W. J., Myers, T. E., Kuehl, N. J. & Taylor, M. T. Selective Modification of Tryptophan Residues in Peptides and Proteins Using a Biomimetic Electron Transfer Process. J. Am. Chem. Soc. 142, 9112-9118 (2020).
[41] Taylor, M. T., Nelson, J. E., Suero, M. G. & Gaunt, M. J. A protein functionalization platform based on selective reactions at methionine residues. Nature 562, 563-568 (2018).
[42] Hahm, H. S. et al. Global targeting of functional tyrosines using sulfur-triazole exchange chemistry. Nat. Chem. Biol. 16, 150-159 (2020).
[43] Bach, K., Beerkens, B. L. H., Zanon, P. R. A. & Hacker, S. M. Light-Activatable, 2,5-Disubstituted Tetrazoles for the Proteome-wide Profiling of Aspartates and Glutamates in Living Bacteria. ACS Cent. Sci. (2020) doi:10.1021/acscentsci.9b01268.
[44] Wei, C. et al. Where Did the Linker-Payload Go? A Quantitative Investigation on the Destination of the Released Linker-Payload from an Antibody-Drug Conjugate with a Maleimide Linker in Plasma. Anal. Chem. 88, 4979-4986 (2016).
[45] Kasper, M. et al. Ethynylphosphonamidates for the Rapid and Cysteine-Selective Generation of Efficacious Antibody-Drug Conjugates. Angew. Chemie Int. Ed. 58, 11631-11636(2019).
[46] Shen, B. Q. et al. Conjugation site modulates the in vivo stability and therapeutic activity of antibody-drug conjugates. Nat. Biotechnol. 30, 184-189 (2012).
[47] Maurais, A. J. & Weerapana, E. Reactive-cysteine profiling for drug discovery. Current Opinion in Chemical Biology vol. 50 29-36 (2019).
[48] Weerapana, E. et al. Quantitative reactivity profiling predicts functional cysteines in proteomes. Nature 468, 790-797 (2010).
[49] Backus, K. M. et al. Proteome-wide covalent ligand discovery in native biological systems. Nature 534, 570-574 (2016).
[50] Zanon, P. R. A., Lewald, L. & Hacker, S. M. Isotopically Labeled Desthiobiotin Azide (isoDTB) Tags Enable Global Profiling of the Bacterial Cysteinome. Angew. Chemie 132, 2851-2858 (2020).
[51] Abegg, D. et al. Proteome-Wide Profiling of Targets of Cysteine reactive Small Molecules by Using Ethynyl Benziodoxolone Reagents. Angew. Chemie 127, 11002-11007 (2015).
[52] Kuljanin, M. et al. Reimagining high-throughput profiling of reactive cysteines for cell-based screening of large electrophile libraries. Nat. Biotechnol. 39, 630-841 (2021).
[53] Stieger, C. E., Franz, L., Kördin, F. & Hackenberger, C. P. R. Diethynyl Phosphinates for Cysteine-Selective Protein Labeling and Disulfide Rebridging. Angew. Chemie Int. Ed. 60, 15359-15364 (2021).
[54] Kasper, M. A. et al. Vinylphosphonites for Staudinger-induced chemoselective peptide cyclization and functionalization. Chem. Sci. 10, 6322-6329 (2019).
[55] Baumann, A. L. et al. Chemically Induced Vinylphosphonothiolate Electrophiles for Thiol-Thiol Bioconjugations. J. Am. Chem. Soc. 142, 9544-9552 (2020).
[56] Park, Y. et al. The mechanism behind enhanced reactivity of unsaturated phosphorus(v) electrophiles towards thiols. Chem. Sci. 12, 8141-8148 (2021).
[57] Schneider, T. et al. Dissecting Ubiquitin Signaling with Linkage-Defined and Protease Resistant Ubiquitin Chains. Angew. Chemie Int. Ed. 53, 12925-12929 (2014).
[58] Dorfer, V. et al. M S Amanda, a universal identification algorithm optimized for high accuracy tandem mass spectra. J. Proteome Res. 13, 3679-3684 (2014).
[59] Götze, M. et al. StavroX-A software for analyzing crosslinked products in protein interaction studies. J. Am. Soc. Mass Spectrom. 23, 76-87 (2012).
[60] Zanon, P. R. A. et al. Profiling the proteome-wide selectivity of diverse electrophiles. https://fragpipe.nesvilab.org (2021) doi:10.26434/CHEMRXIV.14186561.V1.

The invention claimed is:

1. A method of preparing a compound of formula (III) comprising a step of:
reacting a compound of formula (I) 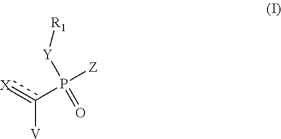
wherein [symbol]
represents a triple bond or a double bond;
V is absent when [symbol] is a triple bond; or
V represents H or C₁-C₈-alkyl when [symbol] is a double bond;
X represents R₃—C when [symbol] is a triple bond; or
X represents 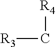
when [symbol] is a double bond;
Y represents O, NR², S, or a bond;
R₁ represents an optionally substituted aliphatic or optionally substituted aromatic residue;
R² represents H or C₁-C₈-alkyl;
R₃ represents H or C₁-C₈-alkyl;
R₄ represents H or C₁-C₈-alkyl; and
Z represents a residue bound to the phosphorus via a carbon atom and comprising a group ●, wherein ● represents an optionally substituted aliphatic or optionally substituted aromatic residue;
with a thiol-containing molecule of formula (II) [symbol]
-SH (II)
wherein [symbol] represents an amino acid, a peptide, a protein, an antibody, a nucleotide, an oligonucleotide, a saccharide, a polysaccharide, a polymer, a small molecule, an optionally substituted C₁-C₈-alkyl, an optionally substituted phenyl, or an optionally substituted aromatic 5- or 6-membered heterocyclic system;
resulting in a compound of formula (III) 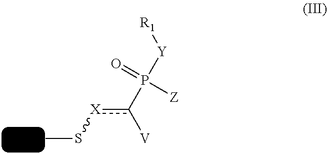
wherein [symbol]
represents a double bond when [symbol] in a compound of formula (I) represents a triple bond; or [symbol]
represents a bond when [symbol] in a compound of formula (I) represents a double bond;
V is absent when [symbol] is a double bond; or
V represents H or C₁-C₈-alkyl when [symbol] is a bond;
X represents R₃—C when [symbol] is a double bond; or
X represents 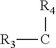
when [symbol] is a bond; and [symbol]
, R₁, R₃, R₄, Y and Z are as defined above.

2. The method according to claim 1, wherein [symbol] represents a triple bond; V is absent; X represents R₃—C; R₃ represents H or C₁-C₈-alkyl, and [symbol] represents a double bond.

3. The method according to claim 1, wherein [symbol] represents a double bond; V represents H or C₁-C₈-alkyl, X represents 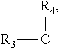
R₃ represents H or C₁-C₈-alkyl, R₄ represents H or C₁-C₈-alkyl, and [symbol] represents a bond.

4. The method according to claim 1,
wherein Z is 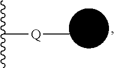
wherein [symbol] indicates the attachment point to the phosphorus and ● is as defined in claim 1; and
Q is a moiety comprising at least three main-chain atoms and a carbon-carbon double bond, wherein at least one of the main chain atoms is selected from the group consisting of S, O or N;
wherein Z is 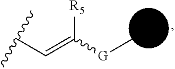
R₅ is H or C₁-C₈-alkyl, G is S, O or NR₁₀ wherein R₁₀ is H or C₁-C₈-alkyl, and ● is as defined in claim 1.

5. (canceled)

6. (canceled)

7. The method according to claim 4, further comprising a preparation of a compound of formula (I), said preparation comprising:
reacting a compound of formula (IV) 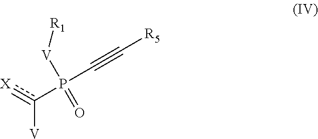
wherein R₁, R₅, [symbol] , V, X and Y are as defined in claim 4,
with ●-GH to form a compound of formula (I), wherein G and ● are as defined in claim 4.

8. The method according to claim 1, wherein
Z is 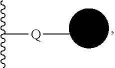
wherein [symbol] indicates the attachment point to the phosphorus and ● is as defined in claim 1; and
Q is a five- or six-membered heterocyclic moiety comprising 1, 2 or 3 heteroatoms independently selected from the group consisting of N, O or S.

9.-10. (canceled)

11. The method according to claim 8, further comprising a preparation of the compound of formula (I), said preparation comprising:
reacting a compound of formula (IV) 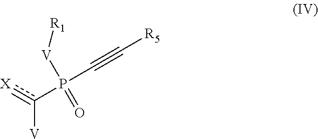
wherein R₅ is H or C₁-C₈-alkyl,
with 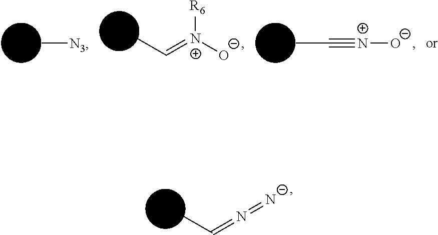
to form a compound of formula (I), wherein ● is as defined in claim 8; R₆ is C₁-C₈-alkyl.

12. The method according to claim 1, wherein
Z is 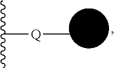
wherein [symbol] indicates the attachment point to the phosphorus and ● is as defined in claim 1; and
Q is a moiety comprising a carbon-carbon triple bond bound to the phosphorus in the compound of formula (I); or
Q is a moiety comprising a carbon-carbon triple bond bound to the phosphorus in formula (I).

13. (canceled)

14. The method according to claim 12, further comprising a preparation of the compound of formula (I), said preparation comprising:
reacting a compound of formula (IV) 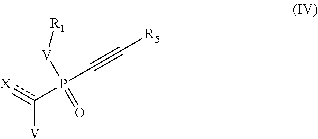
wherein
R₅ is H,
with 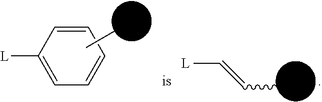
wherein L is halogen (I, Br, Cl) or O-triflate to form a compound of formula (I).

15.-18. (canceled)

19. The method according to claim 1, wherein ● represents a linker, a drug, or a linker-drug conjugate.

20. The method according to claim 1,
wherein [symbol] represents an amino acid, a peptide, a protein, an antibody, a nucleotide, an oligonucleotide, or a small molecule.

21.-24. (canceled)

25. A method of preparing a conjugate of an antibody molecule, said method comprising:
reducing at least one disulfide bridge of an antibody molecule in the presence of a reducing agent; and
reacting said antibody molecule with a compound of formula (IV*) 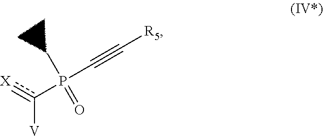
wherein [symbol]
represents a triple bond or a double bond;
V is absent when [symbol] is a triple bond; or
V represents H or C₁-C₈-alkyl when [symbol] is a double bond;
X represents R₃—C when [symbol] is a triple bond; or
X represents 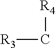
when [symbol] is a double bond;
▴ represents 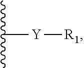
wherein [symbol] indicates the attachment point to the phosphorus; or
▴ represents Z; and
R₁, R₃, R₄, Y and Z are as defined in claim 1, R₅ is H or C₁-C₈-alkyl,
resulting in a conjugate of an antibody molecule comprising at least one moiety of formula 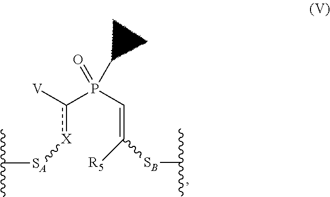
wherein S_{A} and S_{B} are each sulfur atoms of a chain of the antibody molecule; [symbol]
represents a double bond when [symbol] in a compound of formula (IV*) represents a triple bond; or [symbol]
represents a bond when [symbol] in a compound of formula (IV*) represents a double bond;
V is absent when [symbol] is a double bond; or
V represents H or C₁-C₈-alkyl when [symbol] is a bond;
X represents R₃—C when [symbol] is a double bond; or
X represents 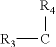
when [symbol] is a bond;
▴ represents 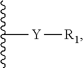
wherein [symbol] indicates the attachment point to the phosphorus; or
▴ represents Z; and
wherein R₁, R₃, R₄, Y and Z are as defined claim 1, and R₅ is H or C₁-C₈-alkyl.

26.-47. (canceled)

48. A compound of formula (I) 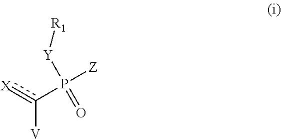
wherein [symbol] , R₁, V, X, Y and Z are as defined in claim 1.

49.-50. (canceled)

51. A compound of formula (III) 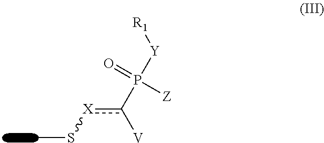
wherein [symbol]
represents a double bond; or [symbol]
represents a bond;
V is absent when [symbol] is a double bond; or
V represents H or C₁-C₈-alkyl when [symbol] is a bond;
X represents R₃—C when [symbol] is a double bond; or
X represents 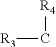
when [symbol] is a bond; and
R₁, R₃, R₄, Y, Z and [symbol] are as defined in claim 1.

52.-65. (canceled)

66. The compound according to claim 48, wherein ● represents a linker, a drug, or a linker-drug conjugate.

67. The compound according to claim 51,
wherein [symbol] represents an amino acid, a peptide, a protein, an antibody, a nucleotide, an oligonucleotide, or a small molecule.

68. The compound according to claim 51, wherein [symbol]
represents an antibody and
● represents a linker, a drug, or a linker-drug conjugate.

69.-70. (canceled)

71. A compound of formula (IIIa) 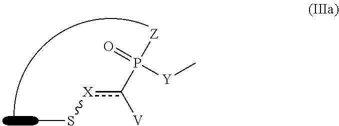
wherein [symbol]
represents a double bond; or [symbol]
represents a bond;
V is absent when [symbol] is a double bond; or
V represents H or C₁-C₈-alkyl when [symbol] is a bond;
X represents R₃—C when [symbol] is a double bond; or
X represents 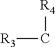
when [symbol] is a bond; and
R₁, R₃, R₄, Y, Z and [symbol] are as defined in claim 1.

72. (canceled)

73. A compound of formula (IV*) 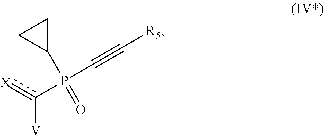
wherein [symbol]
represents a triple bond or a double bond;
V is absent when [symbol] is a triple bond; or
V represents H or C₁-C₈-alkyl when [symbol] is a double bond;
X represents R₃—C when [symbol] is a triple bond;
X represents 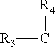
when [symbol] is a double bond;
▴ represents 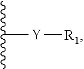
wherein [symbol] indicates the attachment point to the phosphorus; or
▴ represents Z; and
R₁, R₃, R₄, Y and Z are as defined in claim 1; and R₅ is H or C₁-C₈-alkyl.

74.-75. (canceled)

76. A conjugate of an antibody molecule comprising at least one moiety of formula (V) 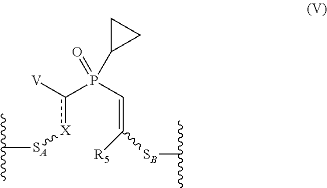
wherein S_{A} and S_{B} are each sulfur atoms of a chain of the antibody molecule; [symbol]
represents a double bond; or [symbol]
represents a bond;
V is absent when [symbol] is a double bond; or
V represents H or C₁-C₈-alkyl when [symbol] is a bond;
X represents R₃—C when [symbol] is a double bond; or
X represents 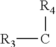
when [symbol] is a bond;
▴ represents 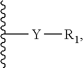
wherein [symbol] indicates the attachment point to the phosphorus; or
▴ represents Z; and
R₁, R₃, R₄, Y and Z are as defined in claim 1; and R₅ is H or C₁-C₈-alkyl.

77.-98. (canceled)
